(19) United States
(12) Patent Application Publication
(10) Pub. No.: US 2017/0296606 A1
(43) Pub. Date: Oct. 19, 2017

(54) 331 NEW FOUND PROTEINS IN PEANUT WITH THEIR SEQUENCE AND THE METHOD OF PRODUCING SUPPLEMENT INCLUDING BEVERAGES ON THE BASIS OF JUICE AND POWDER FROM THE PEANUT

(71) Applicant: Kieu Hoang, Westlake Village, CA (US)

(72) Inventor: Kieu Hoang, Westlake Village, CA (US)

(21) Appl. No.: 15/452,515

(22) Filed: Mar. 7, 2017

Related U.S. Application Data
(60) Provisional application No. 62/304,424, filed on Mar. 7, 2016.
(60) Provisional application No. 62/304,428, filed on Mar. 7, 2016.
(60) Provisional application No. 62/304,434, filed on Mar. 7, 2016.

(51) Int. Cl. A61K 36/48; A61K 9/00; A23L 33/185; A61K 9/20; A61K 38/16; A61K 36/22; A61K 9/48; A61K 9/14

(52) U.S. Cl. CPC A61K 36/48; A61K 9/48; A61K 9/0095; A61K 9/14; A61K 9/20; A61K 38/168; A61K 36/22; A23L 33/185; A23V 2002/00; A61K 2236/13; A61K 2236/15; A61K 2236/53

(57) ABSTRACT

331 new found proteins in peanut with their sequence and the method of producing supplement including beverages on the basis of juice and powder from the peanut. 490 new found proteins in cashew sequence and method of producing food supplements including beverages on the basis of juice and powder from the cashew. 843 new found proteins in edamame (young soy) sequence and method of producing food supplement including beverages on the basis of juice and powder from the edamame (young soy)

### BACKGROUND OF INVENTION

Total of 331 new proteins were found in Peanut and their sequence.

| | | | | # | | | | | |
|---|---|---|---|---|---|---|---|---|---|
| | | | # | Unique | # | # | # | MW | calc |
| Accession | Description | Coverage | Proteins | Peptides | Peptides | PSMs | AAs | [kDa] | pI |
| E9LFE8 | 11S arachin (Fragment) | 45.00 | 1 | 6 | 11 | 30 | 260 | 28.3 | 5.16 |
| | OS = Arachis hypogaea | | | | | | | | |
| | PE = 2 SV = 1 - | | | | | | | | |
| | [E9LFE8_ARAHY] | | | | | | | | |
| E9LFE9 | 11S arachin OS = Arachis | 13.28 | 1 | 3 | 3 | 8 | 256 | 28.3 | 7.46 |
| | hypogaea PE = 2 SV = 1 - | | | | | | | | |
| | [E9LFE9_ARAHY] | | | | | | | | |
| A1E2B0 | 11S seed storage globulin | 44.26 | 1 | 7 | 12 | 40 | 305 | 33.5 | 4.96 |
| | B1 OS = Arachis hypogaea | | | | | | | | |
| | PE = 2 SV = 1 - | | | | | | | | |
| | [A1E2B0_ARAHY] | | | | | | | | |
| A1E2B1 | 11S seed storage globulin | 37.91 | 2 | 9 | 9 | 14 | 277 | 31.2 | 6.24 |
| | B2 OS = Arachis hypogaea | | | | | | | | |
| | PE = 2 SV = 1 - | | | | | | | | |
| | [A1E2B1_ARAHY] | | | | | | | | |
| T2DP01 | 14-3-3 protein | 48.65 | 106 | 6 | 11 | 13 | 259 | 29.1 | 4.75 |
| | OS = Phaseolus vulgaris | | | | | | | | |
| | GN = PHAVU_009G032500g | | | | | | | | |
| | PE = 2 SV = 1 - | | | | | | | | |
| | [T2DP01_PHAVU] | | | | | | | | |
| A0A0B2QZ56 | 14-3-3-like protein A | 24.51 | 82 | 3 | 8 | 10 | 257 | 29.0 | 4.75 |
| | OS = Glycine soja | | | | | | | | |
| | GN = glysoja_027061 PE = 3 | | | | | | | | |
| | SV = 1 - | | | | | | | | |
| | [A0A0B2QZ56_GLYSO] | | | | | | | | |
| A0A0B2SSX7 | 14-3-3-like protein C | 14.29 | 51 | 1 | 3 | 4 | 259 | 29.2 | 4.96 |
| | OS = Glycine soja | | | | | | | | |
| | GN = glysoja_015460 PE = 3 | | | | | | | | |
| | SV = 1 - | | | | | | | | |
| | [A0A0B2SSX7_GLYSO] | | | | | | | | |
| Q2PXN2 | 17.5 kDa class I HSP | 17.61 | 2 | 1 | 2 | 3 | 142 | 16.3 | 6.06 |
| | (Fragment) OS = Arachis | | | | | | | | |
| | hypogaea PE = 2 SV = 1 - | | | | | | | | |
| | [Q2PXN2_ARAHY] | | | | | | | | |
| A0A072UYS2 | 17.6 kDa class I heat | 30.30 | 20 | 1 | 4 | 6 | 231 | 26.5 | 5.83 |
| | shock protein | | | | | | | | |
| | OS = Medicago truncatula | | | | | | | | |
| | GN = MTR_4g088475 | | | | | | | | |
| | PE = 3 SV = 1 - | | | | | | | | |
| | [A0A072UYS2_MEDTR] | | | | | | | | |
| A0A0B2RPZ0 | 18.2 kDa class I heat | 6.94 | 1 | 1 | 1 | 1 | 144 | 16.4 | 5.71 |
| | shock protein OS = Glycine | | | | | | | | |
| | soja GN = glysoja_046553 | | | | | | | | |
| | PE = 3 SV = 1 - | | | | | | | | |
| | [A0A0B2RPZ0_GLYSO] | | | | | | | | |
| P27880 | 18.2 kDa class I heat | 30.38 | 38 | 1 | 5 | 8 | 158 | 18.2 | 6.09 |
| | shock protein | | | | | | | | |
| | OS = Medicago sativa | | | | | | | | |
| | GN = HSP18.2 PE = 2 SV = 1 - | | | | | | | | |
| | [HSP12_MEDSA] | | | | | | | | |
| A0A0B2RC62 | 2,3-bisphosphoglycerate- | 6.26 | 4 | 1 | 2 | 2 | 559 | 60.9 | 5.69 |
| | independent | | | | | | | | |
| | phosphoglycerate mutase | | | | | | | | |
| | OS = Glycine soja | | | | | | | | |
| | GN = glysoja_005186 PE = 4 | | | | | | | | |
| | SV = 1 - | | | | | | | | |
| | [A0A0B2RC62_GLYSO] | | | | | | | | |
| A0A0B2QYR1 | 2,3-dimethylmalate lyase | 7.24 | 10 | 2 | 2 | 2 | 290 | 32.0 | 5.06 |
| | OS = Glycine soja | | | | | | | | |
| | GN = glysoja_027072 PE = 4 | | | | | | | | |
| | SV = 1 - | | | | | | | | |
| | [A0A0B2QYR1_GLYSO] | | | | | | | | |
| G7K3S8 | 20S proteasome beta | 3.94 | 10 | 1 | 1 | 1 | 203 | 22.5 | 6.54 |
| | protein OS = Medicago | | | | | | | | |
| | truncatula | | | | | | | | |
| | GN = MTR_5g006840 | | | | | | | | |
| | PE = 4 SV = 1 - | | | | | | | | |
| | [G7K3S8_MEDTR] | | | | | | | | |
| A0A0B2P437 | 26S protease regulatory | 2.69 | 11 | 1 | 1 | 1 | 334 | 37.0 | 5.29 |
| | subunit 4 like OS = Glycine | | | | | | | | |
| | soja GN = glysoja_002456 | | | | | | | | |
| | PE = 3 SV = 1 - | | | | | | | | |
| | [A0A0B2P437_GLYSO] | | | | | | | | |
| A0A0B2QRD1 | 26S protease regulatory | 17.59 | 52 | 4 | 5 | 5 | 398 | 44.5 | 7.83 |
| | subunit S10B like B | | | | | | | | |
| | OS = Glycine soja | | | | | | | | |
| | GN = glysoja_021983 PE = 3 | | | | | | | | |
| | SV = 1 - | | | | | | | | |
| | [A0A0B2QRD1_GLYSO] | | | | | | | | |
| A0A0B2SL31 | 26S proteasome | 2.59 | 8 | 1 | 1 | 1 | 309 | 34.6 | 6.71 |
| | non-ATPase regulatory | | | | | | | | |
| | subunit 14 OS = Glycine | | | | | | | | |
| | soja GN = glysoja_020249 | | | | | | | | |
| | PE = 4 SV = 1 - | | | | | | | | |
| | [A0A0B2SL31_GLYSO] | | | | | | | | |
| G7LC71 | 26S proteasome regulatory | 5.02 | 9 | 2 | 2 | 2 | 438 | 50.2 | 6.80 |
| | subunit N5 OS = Medicago | | | | | | | | |
| | truncatula | | | | | | | | |
| | GN = MTR_8g092390 | | | | | | | | |
| | PE = 4 SV = 2 - | | | | | | | | |
| | [G7LC71_MEDTR] | | | | | | | | |
| A0A072UXV8 | 26S proteasome regulatory | 2.74 | 8 | 2 | 2 | 2 | 767 | 84.1 | 5.16 |
| | subunit S2 1B | | | | | | | | |
| | OS = Medicago truncatula | | | | | | | | |
| | GN = MTR_4g078780 | | | | | | | | |
| | PE = 4 SV = 1 - | | | | | | | | |
| | [A0A072UXV8_MEDTR] | | | | | | | | |
| Q647G8 | 2S protein 2 OS = Arachis | 42.41 | 2 | 4 | 7 | 24 | 158 | 18.4 | 7.17 |
| | hypogaea PE = 2 SV = 1 - | | | | | | | | |
| | [Q647G8_ARAHY] | | | | | | | | |
| O64458 | 34 kDa maturing seed | 5.28 | 2 | 1 | 1 | 1 | 379 | 42.7 | 5.99 |
| | protein OS = Glycine max | | | | | | | | |
| | GN = P34 PE = 2 SV = 1 - | | | | | | | | |
| | [O64458_SOYBN] | | | | | | | | |
| A0A0B2Q5M9 | 3-hydroxyisobutyryl-CoA | 2.92 | 5 | 1 | 1 | 1 | 377 | 41.0 | 5.94 |
| | hydrolase-like protein 3, | | | | | | | | |
| | mitochondrial OS = Glycine | | | | | | | | |
| | soja GN = glysoja_002687 | | | | | | | | |
| | PE = 4 SV = 1 - | | | | | | | | |
| | [A0A0B2Q5M9_GLYSO] | | | | | | | | |
| E3NYU4 | 3-ketoacyl-CoA thiolase | 15.75 | 1 | 2 | 2 | 2 | 127 | 13.3 | 7.18 |
| | (Fragment) OS = Arachis | | | | | | | | |
| | diogoi PE = 2 SV = 1 - | | | | | | | | |
| | [E3NYU4_9FABA] | | | | | | | | |
| A0A0B2Q968 | 40S ribosomal protein S16 | 18.33 | 16 | 2 | 2 | 3 | 120 | 13.9 | 10.35 |
| | (Fragment) OS = Glycine | | | | | | | | |
| | soja GN = glysoja_002611 | | | | | | | | |
| | PE = 3 SV = 1 - | | | | | | | | |
| | [A0A0B2Q968_GLYSO] | | | | | | | | |
| A0A0B2PMP3 | 40S ribosomal protein S17 | 28.57 | 21 | 3 | 3 | 4 | 105 | 12.4 | 10.18 |
| | (Fragment) OS = Glycine | | | | | | | | |
| | soja GN = glysoja_049953 | | | | | | | | |
| | PE = 3 SV = 1 - | | | | | | | | |
| | [A0A0B2PMP3_GLYSO] | | | | | | | | |
| A0A0B2RS71 | 40S ribosomal protein | 7.38 | 1 | 1 | 1 | 1 | 122 | 13.7 | 9.58 |
| | S20-2 OS = Glycine soja | | | | | | | | |
| | GN = glysoja_004974 PE = 3 | | | | | | | | |
| | SV = 1 - | | | | | | | | |
| | [A0A0B2RS71_GLYSO] | | | | | | | | |
| C6T6P9 | 40S ribosomal protein S21 | 32.10 | 10 | 2 | 2 | 2 | 81 | 9.1 | 6.77 |
| | OS = Glycine max PE = 2 | | | | | | | | |
| | SV = 1 - | | | | | | | | |
| | [C6T6P9_SOYBN] | | | | | | | | |
| B7FMK4 | 40S ribosomal protein S24 | 14.60 | 9 | 1 | 2 | 5 | 137 | 15.7 | 10.64 |
| | OS = Medicago truncatula | | | | | | | | |
| | GN = MTR_1g081410 | | | | | | | | |
| | PE = 2 SV = 1 - | | | | | | | | |
| | [B7FMK4_MEDTR] | | | | | | | | |
| Q6VEV1 | 40S ribosomal protein S25 | 10.64 | 12 | 1 | 1 | 1 | 94 | 10.5 | 10.76 |
| | (Fragment) OS = Glycine | | | | | | | | |
| | max PE = 2 SV = 1 - | | | | | | | | |
| | [Q6VEV1_SOYBN] | | | | | | | | |
| A0A0B2R397 | 40S ribosomal protein S27 | 20.00 | 6 | 1 | 1 | 1 | 85 | 9.6 | 8.73 |
| | OS = Glycine soja | | | | | | | | |
| | GN = glysoja_021427 PE = 3 | | | | | | | | |
| | SV = 1 - | | | | | | | | |
| | [A0A0B2R397_GLYSO] | | | | | | | | |
| A0A0A6ZDX8 | 40S ribosomal protein S3 | 38.52 | 6 | 3 | 8 | 10 | 244 | 27.0 | 9.66 |
| | OS = Arachis hypogaea | | | | | | | | |
| | PE = 2 SV = 1 - | | | | | | | | |
| | [A0A0A6ZDX8_ARAHY] | | | | | | | | |
| A0A0B2QXC5 | 40S ribosomal protein | 28.94 | 25 | 1 | 6 | 7 | 235 | 26.2 | 9.64 |
| | S3-3 OS = Glycine soja | | | | | | | | |
| | GN = glysoja_014052 PE = 3 | | | | | | | | |
| | SV = 1 - | | | | | | | | |
| | [A0A0B2QXC5_GLYSO] | | | | | | | | |
| G8A1J6 | 40S ribosomal protein S3a | 19.26 | 24 | 2 | 5 | 5 | 244 | 28.0 | 9.42 |
| | OS = Medicago truncatula | | | | | | | | |
| | GN = MTR_122s0013 | | | | | | | | |
| | PE = 3 SV = 1 - | | | | | | | | |
| | [G8A1J6_MEDTR] | | | | | | | | |
| A0A0B2P1R0 | 40S ribosomal protein S4 | 9.47 | 19 | 2 | 3 | 4 | 264 | 29.9 | 10.26 |
| | OS = Glycine soja | | | | | | | | |
| | GN = glysoja_039313 PE = 3 | | | | | | | | |
| | SV = 1 - | | | | | | | | |
| | [A0A0B2P1R0_GLYSO] | | | | | | | | |
| V7B774 | 40S ribosomal protein S4 | 7.58 | 10 | 1 | 2 | 4 | 264 | 29.8 | 10.30 |
| | OS = Phaseolus vulgaris | | | | | | | | |
| | GN = PHAVU_008G221000g | | | | | | | | |
| | PE = 3 SV = 1 - | | | | | | | | |
| | [V7B774_PHAVU] | | | | | | | | |
| A0A0B2PT30 | 40S ribosomal protein S6 | 17.81 | 42 | 1 | 4 | 4 | 247 | 28.1 | 10.71 |
| | OS = Glycine soja | | | | | | | | |
| | GN = glysoja_026658 PE = 3 | | | | | | | | |
| | SV = 1 - | | | | | | | | |
| | [A0A0B2PT30_GLYSO] | | | | | | | | |
| T2DPJ8 | 40S ribosomal protein S6 | 17.81 | 27 | 1 | 4 | 4 | 247 | 28.1 | 10.71 |
| | OS = Phaseolus vulgaris | | | | | | | | |
| | GN = PHAVU_008G135700g | | | | | | | | |
| | PE = 2 SV = 1 - | | | | | | | | |
| | [T2DPJ8_PHAVU] | | | | | | | | |
| A0A0B2SW33 | 40S ribosomal protein SA | 13.49 | 16 | 2 | 3 | 3 | 289 | 31.8 | 5.34 |
| | OS = Glycine soja | | | | | | | | |
| | GN = glysoja_031276 PE = 3 | | | | | | | | |
| | SV = 1 - | | | | | | | | |
| | [A0A0B2SW33_GLYSO] | | | | | | | | |
| B7FMD4 | 40S ribosomal S9-like | 7.61 | 4 | 1 | 2 | 2 | 197 | 22.9 | 10.24 |
| | protein OS = Medicago | | | | | | | | |
| | truncatula | | | | | | | | |
| | GN = MTR_3g005430 | | | | | | | | |
| | PE = 2 SV = 1 - | | | | | | | | |
| | [B7FMD4_MEDTR] | | | | | | | | |
| C6TFT6 | 4-hydroxy-4-methyl-2-oxo | 13.51 | 17 | 2 | 2 | 2 | 148 | 15.8 | 6.54 |
| | glutarate aldolase | | | | | | | | |
| | OS = Glycine max PE = 2 | | | | | | | | |
| | SV = 1 - | | | | | | | | |
| | [C6TFT6_SOYBN] | | | | | | | | |
| V5N8C7 | 4-methyl-5(B-hydroxyethyl)- | 6.86 | 6 | 3 | 3 | 3 | 437 | 47.1 | 7.52 |
| | thiazole monophosphate | | | | | | | | |
| | biosynthesis protein | | | | | | | | |
| | OS = Phaseolus vulgaris | | | | | | | | |
| | PE = 2 SV = 1 - | | | | | | | | |
| | [V5N8C7_PHAVU] | | | | | | | | |
| I1KRJ0 | 6,7-dimethyl-8-ribityllumazine | 8.70 | 2 | 1 | 1 | 1 | 230 | 24.2 | 7.20 |
| | synthase OS = Glycine | | | | | | | | |
| | max PE = 3 SV = 1 - | | | | | | | | |
| | [I1KRJ0_SOYBN] | | | | | | | | |
| N1NK96 | 60s Acidic ribosomal | 23.01 | 1 | 2 | 2 | 3 | 113 | 11.4 | 4.51 |
| | protein OS = Arachis | | | | | | | | |
| | duranensis | | | | | | | | |
| | GN = ARAX_ADH18B08- | | | | | | | | |
| | 016 PE = 3 SV = 1 - | | | | | | | | |
| | [N1NK96_9FABA] | | | | | | | | |
| A0A072TL99 | 60S ribosomal L28-like | 8.11 | 3 | 1 | 1 | 1 | 111 | 12.4 | 10.46 |
| | protein OS = Medicago | | | | | | | | |
| | truncatula | | | | | | | | |
| | GN = MTR_8g015570 | | | | | | | | |
| | PE = 4 SV = 1 - | | | | | | | | |
| | [A0A072TL99_MEDTR] | | | | | | | | |
| G7I6N5 | 60S ribosomal L4-like | 12.56 | 15 | 1 | 5 | 7 | 406 | 44.7 | 10.43 |
| | protein OS = Medicago | | | | | | | | |
| | truncatula | | | | | | | | |
| | GN = MTR_1g045410 | | | | | | | | |
| | PE = 4 SV = 1 - | | | | | | | | |
| | [G7I6N5_MEDTR] | | | | | | | | |
| A0A0A6ZDS7 | 60s ribosomal protein L1 | 22.37 | 37 | 8 | 8 | 9 | 389 | 44.3 | 10.20 |
| | OS = Arachis hypogaea | | | | | | | | |
| | PE = 2 SV = 1 - | | | | | | | | |
| | [A0A0A6ZDS7_ARAHY] | | | | | | | | |
| A0A0B2STA3 | 60S ribosomal protein L10 | 23.24 | 27 | 4 | 4 | 5 | 185 | 20.6 | 10.17 |
| | OS = Glycine soja | | | | | | | | |
| | GN = glysoja_045907 PE = 4 | | | | | | | | |
| | SV = 1 - | | | | | | | | |
| | [A0A0B2STA3_GLYSO] | | | | | | | | |
| A0A0B2PQ30 | 60S ribosomal protein L11 | 23.39 | 37 | 4 | 4 | 5 | 171 | 19.6 | 9.95 |
| | OS = Glycine soja | | | | | | | | |
| | GN = glysoja_043929 PE = 3 | | | | | | | | |
| | SV = 1 - | | | | | | | | |
| | [A0A0B2PQ30_GLYSO] | | | | | | | | |
| I3SI88 | 60S ribosomal protein L13 | 4.35 | 1 | 1 | 1 | 1 | 207 | 24.1 | 11.15 |
| | OS = Lotus japonicus PE = 2 | | | | | | | | |
| | SV = 1 - [I3SI88_LOTJA] | | | | | | | | |
| V7CG35 | 60S ribosomal protein L13 | 5.83 | 5 | 1 | 1 | 1 | 206 | 23.7 | 11.00 |
| | OS = Phaseolus vulgaris | | | | | | | | |
| | GN = PHAVU_003G276400g | | | | | | | | |
| | PE = 3 SV = 1 - | | | | | | | | |
| | [V7CG35_PHAVU] | | | | | | | | |
| B7FJC3 | 60S ribosomal protein | 17.42 | 23 | 3 | 3 | 3 | 178 | 21.3 | 10.30 |
| | L18a OS = Medicago | | | | | | | | |
| | truncatula | | | | | | | | |
| | GN = MTR_2g098000 | | | | | | | | |
| | PE = 2 SV = 1 - | | | | | | | | |
| | [B7FJC3_MEDTR] | | | | | | | | |
| A0A0B2PUY1 | 60S ribosomal protein | 12.67 | 5 | 1 | 2 | 2 | 150 | 16.9 | 10.9 |
| | L28-2 OS = Glycine soja | | | | | | | | |
| | GN = glysoja_023376 PE = 4 | | | | | | | | |
| | SV = 1 - | | | | | | | | |
| | [A0A0B2PUY1_GLYSO] | | | | | | | | |
| A0A072TPY7 | 60S ribosomal protein | 18.75 | 14 | 2 | 2 | 2 | 112 | 12.9 | 10.61 |
| | L35a-3 OS = Medicago | | | | | | | | |
| | truncatula | | | | | | | | |
| | GN = MTR_0699s0010 | | | | | | | | |
| | PE = 4 SV = 1 - | | | | | | | | |
| | [A0A072TPY7_MEDTR] | | | | | | | | |
| I3S4I6 | 60S ribosomal protein L36 | 11.63 | 16 | 1 | 1 | 1 | 86 | 9.8 | 11.14 |
| | OS = Lotus japonicus PE = 2 | | | | | | | | |
| | SV = 1 - [I3S4I6_LOTJA] | | | | | | | | |
| A0A072UWZ5 | 60S ribosomal protein | 11.11 | 20 | 1 | 1 | 1 | 63 | 7.4 | 10.01 |
| | L38A OS = Medicago | | | | | | | | |
| | truncatula | | | | | | | | |
| | GN = MTR_4g068040 | | | | | | | | |
| | PE = 3 SV = 1 - | | | | | | | | |
| | [A0A072UWZ5_MEDTR] | | | | | | | | |
| A0A075BIU4 | 60S ribosomal protein | 23.65 | 11 | 4 | 8 | 12 | 406 | 44.6 | 10.43 |
| | L4/L1 OS = Arachis | | | | | | | | |
| | hypogaea PE = 2 SV = 1 - | | | | | | | | |
| | [A0A075BIU4_ARAHY] | | | | | | | | |
| V7CR67 | 60S ribosomal protein L6 | 9.01 | 4 | 1 | 2 | 2 | 233 | 26.1 | 10.15 |
| | OS = Phaseolus vulgaris | | | | | | | | |
| | GN = PHAVU_002G265000g | | | | | | | | |
| | PE = 3 SV = 1 - | | | | | | | | |
| | [V7CR67_PHAVU] | | | | | | | | |
| A0A0B2P7T8 | 60S ribosomal protein L7a | 23.55 | 14 | 1 | 6 | 6 | 259 | 29.4 | 10.20 |
| | OS = Glycine soja | | | | | | | | |
| | GN = glysoja_020395 PE = 4 | | | | | | | | |
| | SV = 1 - | | | | | | | | |
| | [A0A0B2P7T8_GLYSO] | | | | | | | | |
| A0A0B2NYC0 | 60S ribosomal protein | 22.65 | 11 | 1 | 4 | 5 | 234 | 25.0 | 10.87 |
| | L8-3 OS = Glycine soja | | | | | | | | |
| | GN = glysoja_000155 PE = 4 | | | | | | | | |
| | SV = 1 - | | | | | | | | |
| | [A0A0B2NYC0_GLYSO] | | | | | | | | |
| P30707 | 60S ribosomal protein L9 | 25.91 | 20 | 1 | 4 | 4 | 193 | 21.7 | 9.28 |
| | OS = Pisum sativum | | | | | | | | |
| | GN = RPL9 PE = 2 SV = 1 - | | | | | | | | |
| | [RL9_PEA] | | | | | | | | |
| A0A0B2Q9Z6 | 6-phosphogluconate | 13.42 | 15 | 2 | 3 | 3 | 231 | 25.4 | 9.80 |
| | dehydrogenase, | | | | | | | | |
| | decarboxylating | | | | | | | | |
| | OS = Glycine soja | | | | | | | | |
| | GN = glysoja_002861 PE = 4 | | | | | | | | |
| | SV = 1 - | | | | | | | | |
| | [A0A0B2Q9Z6_GLYSO] | | | | | | | | |
| A0A0B2S500 | ABC transporter E family | 7.28 | 8 | 1 | 1 | 1 | 206 | 23.2 | 8.70 |
| | member 2 OS = Glycine | | | | | | | | |
| | soja GN = glysoja_003516 | | | | | | | | |
| | PE = 4 SV = 1 - | | | | | | | | |
| | [A0A0B2S500_GLYSO] | | | | | | | | |
| A0A0B2PFB3 | Abscisic acid receptor | 7.57 | 11 | 1 | 1 | 1 | 185 | 20.9 | 7.01 |
| | PYL9 OS = Glycine soja | | | | | | | | |
| | GN = glysoja_021136 PE = 4 | | | | | | | | |
| | SV = 1 - | | | | | | | | |
| | [A0A0B2PFB3_GLYSO] | | | | | | | | |
| A0A0B2SPA1 | Acetate--CoA ligase ACS, | 2.15 | 6 | 1 | 1 | 1 | 698 | 77.8 | 5.73 |
| | chloroplastic/glyoxysomal | | | | | | | | |
| | OS = Glycine soja | | | | | | | | |
| | GN = glysoja_038956 PE = 3 | | | | | | | | |
| | SV = 1 - | | | | | | | | |
| | [A0A0B2SPA1_GLYSO] | | | | | | | | |
| I1KH72 | Acetyltransferase | 5.49 | 10 | 2 | 2 | 2 | 546 | 59.3 | 7.91 |
| | component of pyruvate | | | | | | | | |
| | dehydrogenase complex | | | | | | | | |
| | OS = Glycine max PE = 3 | | | | | | | | |
| | SV = 1 - | | | | | | | | |
| | [I1KH72_SOYBN] | | | | | | | | |
| G7ITZ5 | Aconitate hydratase | 5.52 | 20 | 2 | 5 | 5 | 979 | 107.0 | 7.72 |
| | OS = Medicago truncatula | | | | | | | | |
| | GN = MTR_2g088360 | | | | | | | | |
| | PE = 3 SV = 1 - | | | | | | | | |
| | [G7ITZ5_MEDTR] | | | | | | | | |
| A0A072UCS0 | Aconitate hydratase | 4.02 | 5 | 1 | 3 | 3 | 797 | 86.7 | 6.70 |
| | OS = Medicago truncatula | | | | | | | | |
| | GN = MTR_5g022940 | | | | | | | | |
| | PE = 3 SV = 1 - | | | | | | | | |
| | [A0A072UCS0_MEDTR] | | | | | | | | |
| V7BVD2 | Aconitate hydratase | 3.76 | 14 | 1 | 4 | 4 | 983 | 107.0 | 7.72 |
| | OS = Phaseolus vulgaris | | | | | | | | |
| | GN = PHAVU_005G115100g | | | | | | | | |
| | PE = 3 SV = 1 - | | | | | | | | |
| | [V7BVD2_PHAVU] | | | | | | | | |
| Q9ZQT0 | Actin (Fragment) | 59.35 | 17 | 1 | 6 | 8 | 155 | 17.1 | 6.19 |
| | OS = Glycyrrhiza echinata | | | | | | | | |
| | GN = GeACT-1 PE = 2 | | | | | | | | |
| | SV = 1 - | | | | | | | | |
| | [Q9ZQT0_GLYEC] | | | | | | | | |
| A8W4W6 | Actin OS = Glycyrrhiza | 36.34 | 62 | 4 | 10 | 12 | 377 | 41.6 | 5.59 |
| | uralensis PE = 2 SV = 1 - | | | | | | | | |
| | [A8W4W6_GLYUR] | | | | | | | | |
| A0A0B2PX46 | Actin-11 OS = Glycine soja | 25.99 | 52 | 2 | 8 | 10 | 377 | 41.8 | 5.40 |
| | GN = glysoja_016190 PE = 3 | | | | | | | | |
| | SV = 1 - | | | | | | | | |
| | [A0A0B2PX46_GLYSO] | | | | | | | | |
| D8KXY1 | Acyl carrier protein | 10.71 | 1 | 1 | 1 | 1 | 140 | 14.9 | 4.94 |
| | OS = Arachis hypogaea | | | | | | | | |
| | GN = ACP1 PE = 2 SV = 1 - | | | | | | | | |
| | [D8KXY1_ARAHY] | | | | | | | | |
| F8SMB9 | Adenosylhomocysteinase | 20.00 | 25 | 9 | 9 | 9 | 485 | 53.2 | 6.01 |
| | OS = Glycine max PE = 2 | | | | | | | | |
| | SV = 1 - | | | | | | | | |
| | [F8SMB9_SOYBN] | | | | | | | | |
| I1LJE0 | Adenylyl | 1.71 | 6 | 1 | 1 | 1 | 469 | 50.6 | 6.80 |
| | cyclase-associated protein | | | | | | | | |
| | OS = Glycine max PE = 3 | | | | | | | | |
| | SV = 1 - [I1LJE0_SOYBN] | | | | | | | | |
| P93469 | ADP-glucose | 2.20 | 8 | 1 | 1 | 6 | 363 | 40.4 | 5.88 |
| | pyrophosphorylase | | | | | | | | |
| | (Fragment) OS = Pisum | | | | | | | | |
| | sativum GN = agpl2 PE = 2 | | | | | | | | |
| | SV = 1 - [P93469_PEA] | | | | | | | | |
| Q5UIP7 | Alcohol dehydrodenase | 5.26 | 2 | 1 | 1 | 1 | 190 | 19.7 | 6.52 |
| | (Fragment) OS = Arachis | | | | | | | | |
| | hypogaea PE = 2 SV = 1 - | | | | | | | | |
| | [Q5UIP7_ARAHY] | | | | | | | | |
| H6U596 | Alcohol dehydrogenase | 16.22 | 2 | 1 | 3 | 6 | 185 | 19.4 | 6.84 |
| | (Fragment) OS = Arachis | | | | | | | | |
| | hypogaea PE = 2 SV = 1 - | | | | | | | | |
| | [H6U596_ARAHY] | | | | | | | | |
| A0A0B2PNL1 | Alcohol dehydrogenase 1 | 15.26 | 52 | 1 | 5 | 8 | 380 | 40.8 | 6.52 |
| | OS = Glycine soja | | | | | | | | |
| | GN = glysoja_044048 PE = 3 | | | | | | | | |
| | SV = 1 - | | | | | | | | |
| | [A0A0B2PNL1_GLYSO] | | | | | | | | |
| Q43015 | Alcohol | 18.95 | 68 | 1 | 6 | 12 | 380 | 40.9 | 6.28 |
| | dehydrogenase-1CN | | | | | | | | |
| | OS = Phaseolus acutifolius | | | | | | | | |
| | PE = 2 SV = 1 - | | | | | | | | |
| | [Q43015_PHAAT] | | | | | | | | |
| A0A0B2SGP7 | Aldo-keto reductase | 6.21 | 23 | 2 | 2 | 2 | 290 | 32.1 | 6.89 |
| | family 4 member C9 | | | | | | | | |
| | OS = Glycine soja | | | | | | | | |
| | GN = glysoja_047846 PE = 4 | | | | | | | | |
| | SV = 1 - | | | | | | | | |
| | [A0A0B2SGP7_GLYSO] | | | | | | | | |
| I3SM26 | Aldose 1-epimerase | 4.84 | 15 | 2 | 2 | 2 | 372 | 40.7 | 9.63 |
| | OS = Lotus japonicus PE = 2 | | | | | | | | |
| | SV = 1 - [I3SM26_LOTJA] | | | | | | | | |
| P43238 | Allergen Ara h 1, clone | 60.54 | 5 | 11 | 45 | 472 | 626 | 71.3 | 7.06 |
| | P41B OS = Arachis | | | | | | | | |
| | hypogaea PE = 1 SV = 1 - | | | | | | | | |
| | [ALL12_ARAHY] | | | | | | | | |
| A0A072TGQ9 | Alpha-1,4 glucan | 4.81 | 18 | 3 | 3 | 3 | 914 | 103.3 | 5.19 |
| | phosphorylase | | | | | | | | |
| | OS = Medicago truncatula | | | | | | | | |
| | GN = MTR_0288s0040 | | | | | | | | |
| | PE = 3 SV = 1 - | | | | | | | | |
| | [A0A072TGQ9_MEDTR] | | | | | | | | |
| A0A072W1R5 | Alpha-glucosidase | 1.08 | 1 | 1 | 1 | 1 | 925 | 103.2 | 6.60 |
| | OS = Medicago truncatula | | | | | | | | |
| | GN = MTR_1g110980 | | | | | | | | |
| | PE = 3 SV = 1 - | | | | | | | | |
| | [A0A072W1R5_MEDTR] | | | | | | | | |
| G7JXA7 | Alpha-glucosidase | 2.16 | 5 | 1 | 2 | 2 | 926 | 102.9 | 6.77 |
| | OS = Medicago truncatula | | | | | | | | |
| | GN = MTR_5g057990 | | | | | | | | |
| | PE = 3 SV = 1 - | | | | | | | | |
| | [G7JXA7_MEDTR] | | | | | | | | |
| Q1KSI0 | Alpha-tubulin (Fragment) | 9.09 | 50 | 1 | 1 | 1 | 132 | 14.5 | 5.27 |
| | OS = Phaseolus vulgaris | | | | | | | | |
| | PE = 2 SV = 1 - | | | | | | | | |
| | [Q1KSI0_PHAVU] | | | | | | | | |
| V7BV13 | Amine oxidase | 1.10 | 6 | 1 | 1 | 1 | 727 | 81.7 | 6.23 |
| | OS = Phaseolus vulgaris | | | | | | | | |
| | GN = PHAVU_005G023500g | | | | | | | | |
| | PE = 3 SV = 1 - | | | | | | | | |
| | [V7BV13_PHAVU] | | | | | | | | |
| V7C7X2 | Aminoacylase-1 | 5.31 | 9 | 2 | 2 | 2 | 452 | 50.5 | 6.13 |
| | OS = Phaseolus vulgaris | | | | | | | | |
| | GN = PHAVU_003G001200g | | | | | | | | |
| | PE = 3 SV = 1 - | | | | | | | | |
| | [V7C7X2_PHAVU] | | | | | | | | |
| A0A0F7GF62 | Annexin OS = Arachis | 53.02 | 30 | 1 | 17 | 23 | 315 | 36.1 | 8.51 |
| | hypogaea GN = AnnAh6 | | | | | | | | |
| | PE = 2 SV = 1 - | | | | | | | | |
| | [A0A0F7GF62_ARAHY] | | | | | | | | |
| A0A0F6VX63 | Annexin OS = Arachis | 48.42 | 14 | 11 | 12 | 15 | 316 | 36.1 | 7.55 |
| | hypogaea PE = 2 SV = 1 - | | | | | | | | |
| | [A0A0F6VX63_ARAHY] | | | | | | | | |
| F6KLJ6 | Annexin OS = Arachis | 53.02 | 29 | 1 | 17 | 24 | 315 | 36.1 | 8.29 |
| | hypogaea var. vulgaris | | | | | | | | |
| | PE = 2 SV = 1 - | | | | | | | | |
| | [F6KLJ6_ARAHY] | | | | | | | | |
| C6T7M2 | Annexin OS = Glycine max | 9.94 | 21 | 1 | 4 | 5 | 312 | 35.8 | 7.91 |
| | PE = 2 SV = 1 - | | | | | | | | |
| | [C6T7M2_SOYBN] | | | | | | | | |
| A5Z1Q5 | Ara d 6 OS = Arachis | 67.59 | 1 | 1 | 12 | 167 | 145 | 16.9 | 6.01 |
| | duranensis GN = Ara d 6 | | | | | | | | |
| | PE = 2 SV = 1 - | | | | | | | | |
| | [A5Z1Q5_9FABA] | | | | | | | | |
| B4XID4 | Ara h 7 allergen | 50.00 | 2 | 4 | 7 | 28 | 164 | 19.3 | 7.90 |
| | OS = Arachis hypogaea | | | | | | | | |
| | PE = 2 SV = 1 - | | | | | | | | |
| | [B4XID4_ARAHY] | | | | | | | | |
| B1PYZ4 | Ara h 8 allergen isoform 3 | 29.30 | 36 | 1 | 4 | 4 | 157 | 16.9 | 5.11 |
| | OS = Arachis hypogaea | | | | | | | | |
| | PE = 3 SV = 1 - | | | | | | | | |
| | [B1PYZ4_ARAHY] | | | | | | | | |
| B0YIU5 | Ara h 8 allergen isoform | 64.71 | 2 | 6 | 6 | 27 | 153 | 16.4 | 5.20 |
| | OS = Arachis hypogaea | | | | | | | | |
| | PE = 2 SV = 1 - | | | | | | | | |
| | [B0YIU5_ARAHY] | | | | | | | | |
| Q6VT83 | Ara h 8 allergen | 29.94 | 37 | 1 | 4 | 4 | 157 | 16.9 | 5.11 |
| | OS = Arachis hypogaea | | | | | | | | |
| | PE = 1 SV = 1 - | | | | | | | | |
| | [Q6VT83_ARAHY] | | | | | | | | |
| A1DZF0 | Arachin 6 OS = Arachis | 47.83 | 1 | 1 | 18 | 400 | 529 | 60.3 | 5.54 |
| | hypogaea PE = 2 SV = 1 - | | | | | | | | |
| | [A1DZF0_ARAHY] | | | | | | | | |
| Q647H4 | Arachin Ahy-1 | 68.10 | 5 | 7 | 25 | 526 | 536 | 61.5 | 5.81 |
| | OS = Arachis hypogaea | | | | | | | | |
| | PE = 2 SV = 1 - | | | | | | | | |
| | [Q647H4_ARAHY] | | | | | | | | |
| Q647H3 | Arachin Ahy-2 | 63.13 | 1 | 1 | 22 | 498 | 537 | 61.5 | 5.73 |
| | OS = Arachis hypogaea | | | | | | | | |
| | PE = 2 SV = 1 - | | | | | | | | |
| | [Q647H3_ARAHY] | | | | | | | | |
| Q647H2 | Arachin Ahy-3 | 36.78 | 1 | 12 | 14 | 104 | 484 | 54.5 | 5.59 |
| | OS = Arachis hypogaea | | | | | | | | |
| | PE = 1 SV = 1 - | | | | | | | | |
| | [AHY3_ARAHY] | | | | | | | | |
| Q5I6T2 | Arachin Ahy-4 | 59.13 | 1 | 3 | 24 | 492 | 531 | 60.7 | 5.48 |
| | OS = Arachis hypogaea | | | | | | | | |
| | PE = 2 SV = 1 - | | | | | | | | |
| | [Q5I6T2_ARAHY] | | | | | | | | |
| B5TYU1 | Arachin Arah3 isoform | 59.81 | 1 | 2 | 25 | 442 | 530 | 60.6 | 5.55 |
| | OS = Arachis hypogaea | | | | | | | | |
| | PE = 1 SV = 1 - | | | | | | | | |
| | [B5TYU1_ARAHY] | | | | | | | | |
| D7RXM7 | Ascorbate peroxidase | 14.84 | 21 | 1 | 1 | 1 | 155 | 16.6 | 5.19 |
| | (Fragment) OS = Vigna | | | | | | | | |
| | mungo PE = 2 SV = 1 - | | | | | | | | |
| | [D7RXM7_VIGMU] | | | | | | | | |
| I1K999 | Aspartate aminotransferase | 4.76 | 32 | 2 | 2 | 2 | 357 | 38.6 | 8.78 |
| | OS = Glycine max PE = 3 | | | | | | | | |
| | SV = 1 - [I1K999_SOYBN] | | | | | | | | |
| G7I885 | Aspartate-tRNA ligase | 6.10 | 6 | 1 | 3 | 3 | 541 | 60.5 | 6.40 |
| | OS = Medicago truncatula | | | | | | | | |
| | GN = MTR_1g014140 | | | | | | | | |
| | PE = 3 SV = 1 - | | | | | | | | |
| | [G7I885_MEDTR] | | | | | | | | |
| A0A0B2PMS7 | Aspartic proteinase | 6.60 | 6 | 2 | 2 | 2 | 288 | 31.7 | 9.31 |
| | nepenthesin-1 OS = Glycine | | | | | | | | |
| | soja GN = glysoja_021841 | | | | | | | | |
| | PE = 4 SV = 1 - | | | | | | | | |
| | [A0A0B2PMS7_GLYSO] | | | | | | | | |
| V5N8Y2 | Aspartic proteinase | 2.49 | 2 | 1 | 1 | 1 | 481 | 51.2 | 7.52 |
| | nepenthesin-1 | | | | | | | | |
| | OS = Phaseolus vulgaris | | | | | | | | |
| | PE = 2 SV = 1 - | | | | | | | | |
| | [V5N8Y2_PHAVU] | | | | | | | | |
| T2DNA5 | Aspartic proteinase | 4.59 | 10 | 2 | 2 | 2 | 436 | 46.7 | 8.94 |
| | nepenthesin-1-like protein | | | | | | | | |
| | OS = Phaseolus vulgaris | | | | | | | | |
| | PE = 2 SV = 1 - | | | | | | | | |
| | [T2DNA5_PHAVU] | | | | | | | | |
| A0A060A425 | Aspartic proteinase | 5.71 | 5 | 3 | 3 | 3 | 508 | 55.3 | 6.14 |
| | OS = Cicer arietinum | | | | | | | | |
| | GN = AP2 PE = 3 SV = 1 - | | | | | | | | |
| | [A0A060A425_CICAR] | | | | | | | | |
| R4IV40 | ATP synthase subunit | 21.40 | 74 | 9 | 9 | 13 | 472 | 50.8 | 7.78 |
| | alpha OS = Vicia faba PE = 3 | | | | | | | | |
| | SV = 1 - [R4IV40_VICFA] | | | | | | | | |
| A0A072W1H5 | ATP synthase subunit beta | 25.22 | 107 | 2 | 10 | 15 | 559 | 59.9 | 6.15 |
| | OS = Medicago truncatula | | | | | | | | |
| | GN = MTR_1g108765 | | | | | | | | |
| | PE = 3 SV = 1 - | | | | | | | | |
| | [A0A072W1H5_MEDTR] | | | | | | | | |
| G7IAW2 | ATP synthase subunit beta | 22.00 | 105 | 1 | 9 | 11 | 559 | 60.0 | 6.02 |
| | OS = Medicago truncatula | | | | | | | | |
| | GN = MTR_1g108770 | | | | | | | | |
| | PE = 3 SV = 2 - | | | | | | | | |
| | [G7IAW2_MEDTR] | | | | | | | | |
| I3S9V0 | ATP-dependent Clp | 12.10 | 4 | 1 | 1 | 1 | 124 | 13.9 | 5.45 |
| | protease proteolytic | | | | | | | | |
| | subunit OS = Lotus | | | | | | | | |
| | japonicus PE = 2 SV = 1 - | | | | | | | | |
| | [I3S9V0_LOTJA] | | | | | | | | |
| Q45W71 | Auxin-repressed protein | 10.74 | 2 | 1 | 1 | 1 | 121 | 13.5 | 9.64 |
| | OS = Arachis hypogaea | | | | | | | | |
| | PE = 2 SV = 1 - | | | | | | | | |
| | [Q45W71_ARAHY] | | | | | | | | |
| Q8RVH5 | Basic 7S globulin 2 | 11.09 | 2 | 1 | 2 | 4 | 433 | 47.2 | 8.25 |
| | OS = Glycine max PE = 1 | | | | | | | | |
| | SV = 1 - [7SBG2_SOYBN] | | | | | | | | |
| A0A0B2P3W9 | Basic 7S globulin | 15.31 | 3 | 1 | 2 | 2 | 307 | 33.1 | 8.37 |
| | OS = Glycine soja | | | | | | | | |
| | GN = glysoja_022697 PE = 4 | | | | | | | | |
| | SV = 1 - | | | | | | | | |
| | [A0A0B2P3W9_GLYSO] | | | | | | | | |
| A0A072UPX8 | B-cell | 13.98 | 6 | 1 | 1 | 1 | 93 | 11.4 | 9.19 |
| | receptor-associated-like | | | | | | | | |
| | protein OS = Medicago | | | | | | | | |
| | truncatula | | | | | | | | |
| | GN = MTR_5g048060 | | | | | | | | |
| | PE = 4 SV = 1 - | | | | | | | | |
| | [A0A072UPX8_MEDTR] | | | | | | | | |
| O22121 | Beta subunit of beta | 14.90 | 9 | 4 | 4 | 8 | 416 | 47.9 | 5.92 |
| | conglycinin (Fragment) | | | | | | | | |
| | OS = Glycine max PE = 2 | | | | | | | | |
| | SV = 2 - | | | | | | | | |
| | [O22121_SOYBN] | | | | | | | | |
| P10538 | Beta-amylase OS = Glycine | 15.12 | 17 | 5 | 5 | 9 | 496 | 56.1 | 5.64 |
| | max GN = BMY1 PE = 1 | | | | | | | | |
| | SV = 3 - [AMYB_SOYBN] | | | | | | | | |
| Q84UB3 | Beta-conglycinin alpha′ | 16.16 | 10 | 2 | 4 | 63 | 396 | 45.0 | 5.82 |
| | subunit (Fragment) | | | | | | | | |
| | OS = Glycine max | | | | | | | | |
| | GN = GM7S PE = 2 SV = 1 - | | | | | | | | |
| | [Q84UB3_SOYBN] | | | | | | | | |
| A0A0B2PJY4 | Beta-galactosidase | 1.20 | 4 | 1 | 1 | 1 | 747 | 83.0 | 6.25 |
| | OS = Glycine soja | | | | | | | | |
| | GN = glysoja_032426 PE = 3 | | | | | | | | |
| | SV = 1 - | | | | | | | | |
| | [A0A0B2PJY4_GLYSO] | | | | | | | | |
| A0A0B2SCY0 | Beta-glucosidase 44 | 5.67 | 188 | 2 | 2 | 2 | 406 | 47.0 | 9.28 |
| | OS = Glycine soja | | | | | | | | |
| | GN = glysoja_027222 PE = 3 | | | | | | | | |
| | SV = 1 - | | | | | | | | |
| | [A0A0B2SCY0_GLYSO] | | | | | | | | |
| D8KXZ5 | Beta-hydroxyacyl-ACP | 22.73 | 20 | 4 | 4 | 4 | 220 | 24.0 | 9.04 |
| | dehydrase 1 OS = Arachis | | | | | | | | |
| | hypogaea GN = HD1 PE = 2 | | | | | | | | |
| | SV = 1 - | | | | | | | | |
| | [D8KXZ5_ARAHY] | | | | | | | | |
| D8KXY8 | Beta-ketoacyl-ACP | 27.23 | 15 | 7 | 7 | 8 | 470 | 50.0 | 8.19 |
| | synthase I-1 OS = Arachis | | | | | | | | |
| | hypogaea GN = KASI-1 | | | | | | | | |
| | PE = 2 SV = 1 - | | | | | | | | |
| | [D8KXY8_ARAHY] | | | | | | | | |
| A0A0B2QWM0 | Bifunctional | 1.40 | 9 | 1 | 1 | 1 | 572 | 62.4 | 6.67 |
| | aspartokinase/homoserine | | | | | | | | |
| | dehydrogenase 2, | | | | | | | | |
| | chloroplastic (Fragment) | | | | | | | | |
| | OS = Glycine soja | | | | | | | | |
| | GN = glysoja_025205 PE = 4 | | | | | | | | |
| | SV = 1 - | | | | | | | | |
| | [A0A0B2QWM0_GLYSO] | | | | | | | | |
| A0A0B2NX61 | Biotin carboxylase 1, | 10.39 | 11 | 5 | 5 | 5 | 539 | 58.9 | 7.50 |
| | chloroplastic OS = Glycine | | | | | | | | |
| | soja GN = glysoja_017556 | | | | | | | | |
| | PE = 4 SV = 1 - | | | | | | | | |
| | [A0A0B2NX61_GLYSO] | | | | | | | | |
| Q587K1 | BiP OS = Glycine max | 19.91 | 32 | 3 | 11 | 16 | 668 | 73.5 | 5.15 |
| | GN = Gm bip PE = 2 SV = 1 - | | | | | | | | |
| | [Q587K1_SOYBN] | | | | | | | | |
| P01066 | Bowman-Birk type | 27.14 | 1 | 1 | 1 | 1 | 70 | 7.6 | 5.29 |
| | proteinase inhibitor A-II | | | | | | | | |
| | OS = Arachis hypogaea | | | | | | | | |
| | PE = 1 SV = 1 - | | | | | | | | |
| | [IBB1_ARAHY] | | | | | | | | |
| G7LGJ8 | Calnexin 2 OS = Medicago | 7.22 | 13 | 1 | 4 | 4 | 540 | 61.8 | 4.97 |
| | truncatula | | | | | | | | |
| | GN = MTR_8g095680 | | | | | | | | |
| | PE = 3 SV = 1 - | | | | | | | | |
| | [G7LGJ8_MEDTR] | | | | | | | | |
| T2DLJ5 | Calnexin-like protein | 6.78 | 16 | 1 | 4 | 5 | 546 | 62.2 | 5.14 |
| | OS = Phaseolus vulgaris | | | | | | | | |
| | PE = 2 SV = 1 - | | | | | | | | |
| | [T2DLJ5_PHAVU] | | | | | | | | |
| G7I9H2 | Calreticulin OS = Medicago | 9.93 | 4 | 1 | 4 | 5 | 423 | 48.4 | 4.56 |
| | truncatula | | | | | | | | |
| | GN = MTR_1g083960 | | | | | | | | |
| | PE = 2 SV = 1 - | | | | | | | | |
| | [G7I9H2_MEDTR] | | | | | | | | |
| A0A072V9I1 | Carboxy-terminal TIM | 5.09 | 1 | 2 | 2 | 3 | 491 | 52.5 | 6.29 |
| | barrel domain enolase | | | | | | | | |
| | OS = Medicago truncatula | | | | | | | | |
| | GN = MTR_2g056190 | | | | | | | | |
| | PE = 3 SV = 1 - | | | | | | | | |
| | [A0A072V9I1_MEDTR] | | | | | | | | |
| H6VUU1 | Catalase (Fragment) | 32.58 | 6 | 2 | 6 | 8 | 264 | 30.3 | 5.71 |
| | OS = Ammopiptanthus | | | | | | | | |
| | mongolicus PE = 2 SV = 1 - | | | | | | | | |
| | [H6VUU1_9FABA] | | | | | | | | |
| A0PG71 | Catalase OS = Lotus | 16.46 | 9 | 2 | 7 | 10 | 492 | 56.9 | 7.15 |
| | japonicus GN = cat1 PE = 3 | | | | | | | | |
| | SV = 1 - [A0PG71_LOTJA] | | | | | | | | |
| C0STY9 | Catalase OS = Pisum | 16.19 | 15 | 1 | 7 | 12 | 494 | 57.1 | 7.23 |
| | sativum GN = PCAT1 PE = 2 | | | | | | | | |
| | SV = 1 - [C0STY9_PEA] | | | | | | | | |
| A0A0B2PPJ8 | Cell division cycle protein | 8.75 | 29 | 1 | 5 | 6 | 720 | 79.8 | 5.17 |
| | 48 like OS = Glycine soja | | | | | | | | |
| | GN = glysoja_016616 PE = 3 | | | | | | | | |
| | SV = 1 - | | | | | | | | |
| | [A0A0B2PPJ8_GLYSO] | | | | | | | | |
| A0A0B2RF11 | Cell division cycle protein | 7.02 | 11 | 1 | 5 | 6 | 812 | 90.5 | 5.26 |
| | 48 like OS = Glycine soja | | | | | | | | |
| | GN = glysoja_017471 PE = 3 | | | | | | | | |
| | SV = 1 - | | | | | | | | |
| | [A0A0B2RF11_GLYSO] | | | | | | | | |
| A0A072ULA4 | Centromere/microtubule- | 2.12 | 1 | 1 | 1 | 1 | 612 | 68.2 | 9.28 |
| | binding protein | | | | | | | | |
| | OS = Medicago truncatula | | | | | | | | |
| | GN = MTR_4g074160 | | | | | | | | |
| | PE = 4 SV = 1 - | | | | | | | | |
| | [A0A072ULA4_MEDTR] | | | | | | | | |
| A0A0B2SUY4 | Chaperone protein ClpB1 | 5.71 | 16 | 4 | 4 | 4 | 911 | 101.3 | 6.28 |
| | OS = Glycine soja | | | | | | | | |
| | GN = glysoja_015881 PE = 3 | | | | | | | | |
| | SV = 1 - | | | | | | | | |
| | [A0A0B2SUY4_GLYSO] | | | | | | | | |
| A0A0B2PRW2 | Chaperonin CPN60-2, | 22.43 | 16 | 1 | 11 | 11 | 575 | 61.1 | 6.24 |
| | mitochondrial OS = Glycine | | | | | | | | |
| | soja GN = glysoja_050185 | | | | | | | | |
| | PE = 3 SV = 1 - | | | | | | | | |
| | [A0A0B2PRW2_GLYSO] | | | | | | | | |
| B4UW50 | Chitinase-like protein | 9.35 | 1 | 1 | 1 | 1 | 139 | 15.8 | 8.15 |
| | (Fragment) OS = Arachis | | | | | | | | |
| | hypogaea PE = 2 SV = 1 - | | | | | | | | |
| | [B4UW50_ARAHY] | | | | | | | | |
| Q5D185 | Chloroplast glutamine | 2.85 | 21 | 1 | 1 | 1 | 281 | 30.8 | 6.43 |
| | synthetase (Fragment) | | | | | | | | |
| | OS = Glycine max GN = GS | | | | | | | | |
| | PE = 2 SV = 1 - | | | | | | | | |
| | [Q5D185_SOYBN] | | | | | | | | |
| E3NYG3 | Cinnamic acid | 18.14 | 6 | 2 | 3 | 3 | 226 | 25.5 | 5.96 |
| | 4-hydroxylase (Fragment) | | | | | | | | |
| | OS = Arachis diogoi PE = 2 | | | | | | | | |
| | SV = 1 - | | | | | | | | |
| | [E3NYG3_9FABA] | | | | | | | | |
| D6R061 | Citrate synthase | 4.44 | 9 | 2 | 2 | 2 | 518 | 56.9 | 9.19 |
| | OS = Medicago sativa PE = 2 | | | | | | | | |
| | SV = 1 - | | | | | | | | |
| | [D6R061_MEDSA] | | | | | | | | |
| B4UW51 | Class II small heat shock | 7.75 | 18 | 1 | 1 | 1 | 129 | 14.5 | 8.94 |
| | protein Le-HSP17.6 | | | | | | | | |
| | (Fragment) OS = Arachis | | | | | | | | |
| | hypogaea PE = 2 SV = 1 - | | | | | | | | |
| | [B4UW51_ARAHY] | | | | | | | | |
| I1MZ13 | Clathrin heavy chain | 6.35 | 33 | 8 | 8 | 9 | 1700 | 192.8 | 5.53 |
| | OS = Glycine max PE = 3 | | | | | | | | |
| | SV = 2 - | | | | | | | | |
| | [I1MZ13_SOYBN] | | | | | | | | |
| A0A0B2PLS8 | Clathrin light chain 1 | 7.73 | 10 | 1 | 1 | 1 | 207 | 23.6 | 9.11 |
| | OS = Glycine soja | | | | | | | | |
| | GN = glysoja_014307 PE = 4 | | | | | | | | |
| | SV = 1 - | | | | | | | | |
| | [A0A0B2PLS8_GLYSO] | | | | | | | | |
| A0A072UGQ1 | Coatomer subunit beta | 3.89 | 9 | 3 | 3 | 3 | 951 | 106.0 | 5.87 |
| | OS = Medicago truncatula | | | | | | | | |
| | GN = MTR_4g008250 | | | | | | | | |
| | PE = 4 SV = 1 - | | | | | | | | |
| | [A0A072UGQ1_MEDTR] | | | | | | | | |
| A0A0B2PVA2 | Coatomer subunit delta | 3.94 | 16 | 1 | 2 | 2 | 507 | 55.9 | 5.59 |
| | OS = Glycine soja | | | | | | | | |
| | GN = glysoja_015352 PE = 4 | | | | | | | | |
| | SV = 1 - | | | | | | | | |
| | [A0A0B2PVA2_GLYSO] | | | | | | | | |
| D8KZF4 | Cobalamine-independent | 16.18 | 1 | 1 | 1 | 1 | 68 | 7.7 | 4.31 |
| | methionine synthase | | | | | | | | |
| | (Fragment) OS = Arachis | | | | | | | | |
| | diogoi PE = 2 SV = 1 - | | | | | | | | |
| | [D8KZF4_9FABA] | | | | | | | | |
| Q6PSU6 | Conarachin (Fragment) | 62.71 | 2 | 1 | 22 | 323 | 303 | 34.1 | 5.12 |
| | OS = Arachis hypogaea | | | | | | | | |
| | PE = 2 SV = 1 - | | | | | | | | |
| | [Q6PSU6_ARAHY] | | | | | | | | |
| Q6PSU3 | Conarachin (Fragment) | 65.17 | 5 | 3 | 44 | 481 | 580 | 66.5 | 7.17 |
| | OS = Arachis hypogaea | | | | | | | | |
| | PE = 4 SV = 1 - | | | | | | | | |
| | [Q6PSU3_ARAHY] | | | | | | | | |
| Q647H1 | Conarachin OS = Arachis | 39.73 | 2 | 28 | 28 | 89 | 662 | 75.9 | 5.40 |
| | hypogaea PE = 2 SV = 1 - | | | | | | | | |
| | [Q647H1_ARAHY] | | | | | | | | |
| A8VT44 | Conglutin OS = Arachis | 73.75 | 2 | 6 | 13 | 84 | 160 | 18.8 | 6.05 |
| | duranensis GN = ara d 2 | | | | | | | | |
| | PE = 4 SV = 1 - | | | | | | | | |
| | [A8VT44_9FABA] | | | | | | | | |
| Q647G9 | Conglutin OS = Arachis | 67.59 | 2 | 1 | 12 | 160 | 145 | 16.9 | 6.52 |
| | hypogaea PE = 1 SV = 1 - | | | | | | | | |
| | [CONG_ARAHY] | | | | | | | | |
| Q6PSU2 | Conglutin-7 OS = Arachis | 62.21 | 1 | 4 | 11 | 96 | 172 | 20.1 | 6.34 |
| | hypogaea PE = 1 SV = 2 - | | | | | | | | |
| | [CONG7_ARAHY] | | | | | | | | |
| G7IF81 | CS domain protein | 6.01 | 1 | 1 | 1 | 1 | 183 | 20.1 | 4.54 |
| | OS = Medicago truncatula | | | | | | | | |
| | GN = MTR_1g088700 | | | | | | | | |
| | PE = 4 SV = 2 - | | | | | | | | |
| | [G7IF81_MEDTR] | | | | | | | | |
| A0A060D9Z7 | Cte peptide ligase | 2.70 | 1 | 1 | 1 | 2 | 482 | 53.4 | 6.40 |
| | OS = Clitoria ternatea PE = 2 | | | | | | | | |
| | SV = 1 - | | | | | | | | |
| | [A0A060D9Z7_CLITE] | | | | | | | | |
| G7KC87 | Cupin family protein | 1.00 | 2 | 1 | 1 | 1 | 702 | 82.7 | 5.38 |
| | OS = Medicago truncatula | | | | | | | | |
| | GN = MTR_5g019780 | | | | | | | | |
| | PE = 4 SV = 2 - | | | | | | | | |
| | [G7KC87_MEDTR] | | | | | | | | |
| E3NYT5 | Cu—Zn superoxide | 27.78 | 27 | 3 | 3 | 4 | 108 | 10.9 | 5.34 |
| | dismutase (Fragment) | | | | | | | | |
| | OS = Arachis diogoi PE = 2 | | | | | | | | |
| | SV = 1 - | | | | | | | | |
| | [E3NYT5_9FABA] | | | | | | | | |
| B7FJN7 | Cystathionine | 22.93 | 11 | 4 | 4 | 4 | 205 | 22.6 | 9.17 |
| | beta-synthase (CBS) | | | | | | | | |
| | family protein | | | | | | | | |
| | OS = Medicago truncatula | | | | | | | | |
| | GN = MTR_5g076080 | | | | | | | | |
| | PE = 2 SV = 1 - | | | | | | | | |
| | [B7FJN7_MEDTR] | | | | | | | | |
| B5SV85 | Cysteine protease-like | 2.69 | 3 | 1 | 1 | 1 | 335 | 36.9 | 6.30 |
| | protein (Fragment) | | | | | | | | |
| | OS = Robinia pseudoacacia | | | | | | | | |
| | PE = 2 SV = 1 - | | | | | | | | |
| | [B5SV85_ROBPS] | | | | | | | | |
| T2DPA9 | Cysteine synthase | 12.92 | 13 | 3 | 3 | 3 | 325 | 34.4 | 5.80 |
| | OS = Phaseolus vulgaris | | | | | | | | |
| | PE = 2 SV = 1 - | | | | | | | | |
| | [T2DPA9_PHAVU] | | | | | | | | |
| C6THW5 | Cytochrome b-c1 complex | 7.95 | 7 | 2 | 2 | 2 | 264 | 29.2 | 8.44 |
| | subunit Rieske, | | | | | | | | |
| | mitochondrial OS = Glycine | | | | | | | | |
| | max PE = 2 SV = 1 - | | | | | | | | |
| | [C6THW5_SOYBN] | | | | | | | | |
| Q7ITY5 | Cytochrome c oxidase | 3.95 | 10 | 1 | 1 | 1 | 253 | 29.0 | 5.22 |
| | subunit 2 OS = Pseudeminia | | | | | | | | |
| | comosa GN = cox2 PE = 2 | | | | | | | | |
| | SV = 1 - [Q7ITY5_9FABA] | | | | | | | | |
| A0A0B2RLP6 | Cytochrome c oxidase | 10.86 | 6 | 2 | 2 | 2 | 175 | 19.5 | 4.39 |
| | subunit 6b-1 OS = Glycine | | | | | | | | |
| | soja GN = glysoja_032246 | | | | | | | | |
| | PE = 4 SV = 1 - | | | | | | | | |
| | [A0A0B2RLP6_GLYSO] | | | | | | | | |
| A0A0B2NXZ0 | Cytochrome c1-1, heme | 13.14 | 16 | 1 | 1 | 2 | 137 | 15.7 | 8.85 |
| | protein, mitochondrial | | | | | | | | |
| | OS = Glycine soja | | | | | | | | |
| | GN = glysoja_034621 PE = 4 | | | | | | | | |
| | SV = 1 - | | | | | | | | |
| | [A0A0B2NXZ0_GLYSO] | | | | | | | | |
| G7JGP9 | Cytosol aminopeptidase | 8.82 | 13 | 3 | 3 | 3 | 567 | 59.6 | 7.71 |
| | family protein | | | | | | | | |
| | OS = Medicago truncatula | | | | | | | | |
| | GN = MTR_4g130860 | | | | | | | | |
| | PE = 3 SV = 1 - | | | | | | | | |
| | [G7JGP9_MEDTR] | | | | | | | | |
| A0A0B2S6P2 | D-3-phosphoglycerate | 2.08 | 4 | 1 | 1 | 1 | 432 | 45.9 | 5.69 |
| | dehydrogenase, | | | | | | | | |
| | chloroplastic OS = Glycine | | | | | | | | |
| | soja GN = glysoja_020068 | | | | | | | | |
| | PE = 4 SV = 1 - | | | | | | | | |
| | [A0A0B2S6P2_GLYSO] | | | | | | | | |
| A0A0K1LWQ5 | Dehydroascorbate | 15.29 | 8 | 2 | 2 | 2 | 157 | 17.5 | 5.10 |
| | reductase (Fragment) | | | | | | | | |
| | OS = Trifolium repens | | | | | | | | |
| | GN = DR PE = 4 SV = 1 - | | | | | | | | |
| | [A0A0K1LWQ5_TRIRP] | | | | | | | | |
| A0A0B2QMF0 | Delta-1-pyrroline-5-carboxylate | 2.25 | 7 | 1 | 1 | 1 | 621 | 67.4 | 5.87 |
| | synthase (Fragment) | | | | | | | | |
| | OS = Glycine soja | | | | | | | | |
| | GN = glysoja_009210 PE = 3 | | | | | | | | |
| | SV = 1 - | | | | | | | | |
| | [A0A0B2QMF0_GLYSO] | | | | | | | | |
| A2Q666 | Desiccation PCC13-like | 2.34 | 2 | 1 | 1 | 2 | 299 | 32.2 | 7.20 |
| | protein OS = Medicago | | | | | | | | |
| | truncatula | | | | | | | | |
| | GN = MTR_2g033520 | | | | | | | | |
| | PE = 4 SV = 1 - | | | | | | | | |
| | [A2Q666_MEDTR] | | | | | | | | |
| Q2PXN9 | Dessication protectant | 19.73 | 1 | 1 | 3 | 3 | 147 | 16.1 | 5.90 |
| | protein LEA 14 | | | | | | | | |
| | (Fragment) OS = Arachis | | | | | | | | |
| | hypogaea PE = 2 SV = 1 - | | | | | | | | |
| | [Q2PXN9_ARAHY] | | | | | | | | |
| G7KKA1 | Disease resistance protein | 0.88 | 2 | 1 | 1 | 1 | 1246 | 142.4 | 6.62 |
| | (TIR-NBS-LRR class) | | | | | | | | |
| | OS = Medicago truncatula | | | | | | | | |
| | GN = MTR_6g077640 | | | | | | | | |
| | PE = 4 SV = 2 - | | | | | | | | |
| | [G7KKA1_MEDTR] | | | | | | | | |
| G7KJ66 | Disease resistance protein | 0.99 | 1 | 1 | 1 | 1 | 1011 | 116.8 | 6.70 |
| | (TIR-NBS-LRR class), | | | | | | | | |
| | putative OS = Medicago | | | | | | | | |
| | truncatula | | | | | | | | |
| | GN = MTR_6g074780 | | | | | | | | |
| | PE = 4 SV = 2 - | | | | | | | | |
| | [G7KJ66_MEDTR] | | | | | | | | |
| A0A072TVN2 | Dynamin-like protein 1E | 2.46 | 6 | 1 | 1 | 1 | 610 | 68.2 | 8.03 |
| | OS = Medicago truncatula | | | | | | | | |
| | GN = MTR_8g099550 | | | | | | | | |
| | PE = 3 SV = 1 - | | | | | | | | |
| | [A0A072TVN2_MEDTR] | | | | | | | | |
| G7KVS0 | E1 subunit-like | 3.71 | 11 | 3 | 3 | 3 | 1023 | 115.7 | 6.95 |
| | 2-oxoglutarate | | | | | | | | |
| | dehydrogenase | | | | | | | | |
| | OS = Medicago truncatula | | | | | | | | |
| | GN = MTR_7g023770 | | | | | | | | |
| | PE = 4 SV = 2 - | | | | | | | | |
| | [G7KVS0_MEDTR] | | | | | | | | |
| A0EVX1 | EBP1 | 15.44 | 7 | 3 | 5 | 5 | 395 | 43.5 | 6.33 |
| | OS = Ammopiptanthus | | | | | | | | |
| | mongolicus PE = 2 SV = 1 - | | | | | | | | |
| | [A0EVX1_9FABA] | | | | | | | | |
| E1CKY6 | Elongation factor 1 beta | 24.78 | 33 | 1 | 5 | 7 | 230 | 24.9 | 4.65 |
| | OS = Vigna unguiculata | | | | | | | | |
| | GN = VuEF1b PE = 2 SV = 1 - | | | | | | | | |
| | [E1CKY6_VIGUN] | | | | | | | | |
| A0A0B2Q4Z1 | Elongation factor 1-alpha | 22.37 | 27 | 2 | 8 | 16 | 447 | 49.2 | 9.07 |
| | OS = Glycine soja | | | | | | | | |
| | GN = glysoja_032479 PE = 3 | | | | | | | | |
| | SV = 1 - | | | | | | | | |
| | [A0A0B2Q4Z1_GLYSO] | | | | | | | | |
| G7KN61 | Elongation factor 1-alpha | 17.90 | 26 | 1 | 7 | 21 | 447 | 49.2 | 9.07 |
| | OS = Medicago truncatula | | | | | | | | |
| | GN = MTR_6g021800 | | | | | | | | |
| | PE = 3 SV = 2 - | | | | | | | | |
| | [G7KN61_MEDTR] | | | | | | | | |
| A0A072V7Q9 | Elongation factor 1-beta | 22.08 | 36 | 1 | 5 | 7 | 231 | 25.3 | 4.54 |
| | OS = Medicago truncatula | | | | | | | | |
| | GN = MTR_2g461200 | | | | | | | | |
| | PE = 3 SV = 1 - | | | | | | | | |
| | [A0A072V7Q9_MEDTR] | | | | | | | | |
| G7J2G7 | Elongation factor Tu | 4.50 | 1 | 1 | 1 | 1 | 311 | 33.9 | 8.47 |
| | (EF-Tu) GTP-binding | | | | | | | | |
| | family protein | | | | | | | | |
| | OS = Medicago truncatula | | | | | | | | |
| | GN = MTR_3g072130 | | | | | | | | |
| | PE = 4 SV = 1 - | | | | | | | | |
| | [G7J2G7_MEDTR] | | | | | | | | |
| A0A0B2RYP7 | Elongation factor Tu | 7.18 | 5 | 1 | 2 | 2 | 390 | 42.5 | 6.33 |
| | (Fragment) OS = Glycine | | | | | | | | |
| | soja GN = glysoja_007256 | | | | | | | | |
| | PE = 3 SV = 1 - | | | | | | | | |
| | [A0A0B2RYP7_GLYSO] | | | | | | | | |
| A0A0B2SLH4 | Embryonic protein DC-8 | 7.35 | 4 | 1 | 1 | 1 | 449 | 48.8 | 6.42 |
| | OS = Glycine soja | | | | | | | | |
| | GN = glysoja_036852 PE = 4 | | | | | | | | |
| | SV = 1 - | | | | | | | | |
| | [A0A0B2SLH4_GLYSO] | | | | | | | | |
| Q06013 | Endochitinase 1B | 17.39 | 2 | 1 | 1 | 1 | 46 | 4.7 | 8.24 |
| | (Fragment) OS = Arachis | | | | | | | | |
| | hypogaea PE = 2 SV = 1 - | | | | | | | | |
| | [CHIB_ARAHY] | | | | | | | | |
| Q27SZ6 | Endoglucanase (Fragment) | 5.30 | 55 | 3 | 3 | 3 | 604 | 66.7 | 8.59 |
| | OS = Glycine max | | | | | | | | |
| | GN = Cel12 PE = 2 SV = 1 - | | | | | | | | |
| | [Q27SZ6_SOYBN] | | | | | | | | |
| K7MIR3 | Enolase OS = Glycine max | 21.25 | 8 | 3 | 5 | 8 | 353 | 38.3 | 6.80 |
| | PE = 3 SV = 1 - | | | | | | | | |
| | [K7MIR3_SOYBN] | | | | | | | | |
| Q9M434 | Enolase OS = Lupinus | 16.44 | 2 | 2 | 4 | 13 | 444 | 47.8 | 5.25 |
| | luteus PE = 2 SV = 1 - | | | | | | | | |
| | [Q9M434_LUPLU] | | | | | | | | |
| D8KXZ6 | Enoyl-ACP reductase 1-1 | 11.57 | 23 | 1 | 4 | 4 | 389 | 41.4 | 8.56 |
| | OS = Arachis hypogaea | | | | | | | | |
| | GN = ENR1-1 PE = 2 SV = 1 - | | | | | | | | |
| | [D8KXZ6_ARAHY] | | | | | | | | |
| D8KXZ8 | Enoyl-ACP reductase 1-3 | 11.57 | 23 | 1 | 4 | 4 | 389 | 41.4 | 8.56 |
| | OS = Arachis hypogaea | | | | | | | | |
| | GN = ENR1-3 PE = 2 SV = 1 - | | | | | | | | |
| | [D8KXZ8_ARAHY] | | | | | | | | |
| A0A0B2PBC5 | Eukaryotic initiation factor | 11.38 | 51 | 5 | 5 | 5 | 413 | 46.7 | 5.68 |
| | 4A-15 OS = Glycine soja | | | | | | | | |
| | GN = glysoja_040135 PE = 3 | | | | | | | | |
| | SV = 1 - | | | | | | | | |
| | [A0A0B2PBC5_GLYSO] | | | | | | | | |
| A0A0B2Q2D0 | Eukaryotic initiation factor | 1.28 | 4 | 1 | 1 | 1 | 784 | 86.2 | 8.15 |
| | iso-4F subunit p82-34 | | | | | | | | |
| | OS = Glycine soja | | | | | | | | |
| | GN = glysoja_048435 PE = 4 | | | | | | | | |
| | SV = 1 - | | | | | | | | |
| | [A0A0B2Q2D0_GLYSO] | | | | | | | | |
| A0A072UM30 | Eukaryotic peptide chain | 2.29 | 15 | 1 | 1 | 1 | 436 | 48.9 | 5.72 |
| | release factor subunit 1-3 | | | | | | | | |
| | OS = Medicago truncatula | | | | | | | | |
| | GN = MTR_6g478120 | | | | | | | | |
| | PE = 4 SV = 1 - | | | | | | | | |
| | [A0A072UM30_MEDTR] | | | | | | | | |
| G7J950 | Eukaryotic translation | 1.26 | 1 | 1 | 1 | 1 | 716 | 82.1 | 5.12 |
| | initiation factor 3 subunit | | | | | | | | |
| | B OS = Medicago | | | | | | | | |
| | truncatula | | | | | | | | |
| | GN = MTR_3g105430 | | | | | | | | |
| | PE = 3 SV = 2 - | | | | | | | | |
| | [G7J950_MEDTR] | | | | | | | | |
| C6T751 | Eukaryotic translation | 3.41 | 7 | 1 | 1 | 1 | 323 | 37.9 | 6.54 |
| | initiation factor 3 subunit | | | | | | | | |
| | E OS = Glycine max PE = 2 | | | | | | | | |
| | SV = 1 - | | | | | | | | |
| | [C6T751_SOYBN] | | | | | | | | |
| I3SXR7 | Eukaryotic translation | 4.37 | 9 | 1 | 1 | 1 | 229 | 25.4 | 8.59 |
| | initiation factor 3 subunit | | | | | | | | |
| | G OS = Medicago | | | | | | | | |
| | truncatula PE = 2 SV = 1 - | | | | | | | | |
| | [I3SXR7_MEDTR] | | | | | | | | |
| K7MV98 | Eukaryotic translation | 6.23 | 5 | 1 | 1 | 1 | 257 | 29.2 | 4.60 |
| | initiation factor 3 subunit | | | | | | | | |
| | H OS = Glycine max PE = 3 | | | | | | | | |
| | SV = 1 - | | | | | | | | |
| | [K7MV98_SOYBN] | | | | | | | | |
| Q6TEC2 | Eukaryotic translation | 10.89 | 4 | 1 | 1 | 1 | 101 | 11.6 | 7.28 |
| | initiation factor 4E isoform | | | | | | | | |
| | (Fragment) OS = Pisum | | | | | | | | |
| | sativum GN = eIF(iso)4E | | | | | | | | |
| | PE = 2 SV = 1 - | | | | | | | | |
| | [Q6TEC2_PEA] | | | | | | | | |
| A0A072VB87 | Eukaryotic translation | 8.91 | 37 | 1 | 1 | 1 | 101 | 11.2 | 5.48 |
| | initiation factor SUI1 | | | | | | | | |
| | OS = Medicago truncatula | | | | | | | | |
| | GN = MTR_2g083180 | | | | | | | | |
| | PE = 4 SV = 1 - | | | | | | | | |
| | [A0A072VB87_MEDTR] | | | | | | | | |
| A0A0B2PS04 | Ferritin OS = Glycine soja | 3.50 | 3 | 1 | 1 | 2 | 257 | 28.7 | 6.18 |
| | GN = glysoja_037032 PE = 3 | | | | | | | | |
| | SV = 1 - | | | | | | | | |
| | [A0A0B2PS04_GLYSO] | | | | | | | | |
| B7FK36 | Ferritin OS = Medicago | 9.77 | 30 | 1 | 2 | 3 | 256 | 28.8 | 6.60 |
| | truncatula PE = 2 SV = 1 - | | | | | | | | |
| | [B7FK36_MEDTR] | | | | | | | | |
| O65100 | Ferritin-3, chloroplastic | 7.42 | 20 | 1 | 2 | 2 | 256 | 28.4 | 5.80 |
| | OS = Vigna unguiculata | | | | | | | | |
| | PE = 2 SV = 1 - | | | | | | | | |
| | [FRI3_VIGUN] | | | | | | | | |
| B4UW72 | Fiber protein Fb15 | 9.38 | 1 | 1 | 1 | 1 | 96 | 11.2 | 8.65 |
| | (Fragment) OS = Arachis | | | | | | | | |
| | hypogaea PE = 2 SV = 1 - | | | | | | | | |
| | [B4UW72_ARAHY] | | | | | | | | |
| D2DWA6 | Formate dehydrogenase | 10.24 | 9 | 1 | 4 | 4 | 381 | 41.9 | 7.84 |
| | OS = Phaseolus vulgaris | | | | | | | | |
| | PE = 3 SV = 1 - | | | | | | | | |
| | [D2DWA6_PHAVU] | | | | | | | | |
| G7K8Y3 | Formate-tetrahydrofolate | 5.74 | 10 | 2 | 2 | 2 | 627 | 66.6 | 6.80 |
| | ligase OS = Medicago | | | | | | | | |
| | truncatula | | | | | | | | |
| | GN = MTR_5g059390 | | | | | | | | |
| | PE = 3 SV = 2 - | | | | | | | | |
| | [G7K8Y3_MEDTR] | | | | | | | | |
| A0A0B2RJC2 | Fructokinase-2 | 22.58 | 37 | 1 | 5 | 5 | 310 | 33.5 | 5.26 |
| | OS = Glycine soja | | | | | | | | |
| | GN = glysoja_005347 PE = 3 | | | | | | | | |
| | SV = 1 - | | | | | | | | |
| | [A0A0B2RJC2_GLYSO] | | | | | | | | |
| T2B9M0 | Fructose-bisphosphate | 47.77 | 24 | 6 | 11 | 17 | 358 | 38.4 | 7.20 |
| | aldolase OS = Arachis | | | | | | | | |
| | hypogaea PE = 2 SV = 1 - | | | | | | | | |
| | [T2B9M0_ARAHY] | | | | | | | | |
| A0A072VQ58 | Fructose-bisphosphate | 14.83 | 34 | 1 | 4 | 4 | 391 | 42.6 | 8.29 |
| | aldolase OS = Medicago | | | | | | | | |
| | truncatula | | | | | | | | |
| | GN = MTR_1g109200 | | | | | | | | |
| | PE = 3 SV = 1 - | | | | | | | | |
| | [A0A072VQ58_MEDTR] | | | | | | | | |
| V7BET4 | Fructose-bisphosphate | 14.40 | 42 | 1 | 4 | 4 | 382 | 41.8 | 7.75 |
| | aldolase OS = Phaseolus | | | | | | | | |
| | vulgaris | | | | | | | | |
| | GN = PHAVU_007G033800g | | | | | | | | |
| | PE = 3 SV = 1 - | | | | | | | | |
| | [V7BET4_PHAVU] | | | | | | | | |
| O65735 | Fructose-bisphosphate | 18.66 | 25 | 1 | 5 | 7 | 359 | 38.4 | 6.65 |
| | aldolase, cytoplasmic | | | | | | | | |
| | isozyme OS = Cicer | | | | | | | | |
| | arietinum GN = ALDC | | | | | | | | |
| | PE = 2 SV = 1 - | | | | | | | | |
| | [ALF_CICAR] | | | | | | | | |
| A0A0B2REJ2 | Fumarylacetoacetase | 2.38 | 6 | 1 | 1 | 1 | 421 | 45.8 | 6.28 |
| | OS = Glycine soja | | | | | | | | |
| | GN = glysoja_009962 PE = 4 | | | | | | | | |
| | SV = 1 - | | | | | | | | |
| | [A0A0B2REJ2_GLYSO] | | | | | | | | |
| G7JJA2 | Fungal proteinase A, | 3.08 | 7 | 2 | 2 | 2 | 519 | 55.9 | 6.21 |
| | aspartic proteinase | | | | | | | | |
| | superfamily protein | | | | | | | | |
| | OS = Medicago truncatula | | | | | | | | |
| | GN = MTR_4g132420 | | | | | | | | |
| | PE = 3 SV = 1 - | | | | | | | | |
| | [G7JJA2_MEDTR] | | | | | | | | |
| Q38711 | Galactose-binding lectin | 42.07 | 1 | 3 | 6 | 89 | 271 | 29.1 | 5.64 |
| | (Fragment) OS = Arachis | | | | | | | | |
| | hypogaea GN = lec PE = 2 | | | | | | | | |
| | SV = 1 - | | | | | | | | |
| | [Q38711_ARAHY] | | | | | | | | |
| Q43373 | Galactose-binding lectin | 11.96 | 1 | 2 | 2 | 5 | 276 | 29.5 | 6.55 |
| | OS = Arachis hypogaea | | | | | | | | |
| | GN = lec PE = 2 SV = 1 - | | | | | | | | |
| | [Q43373_ARAHY] | | | | | | | | |
| A0A072U593 | Gamma carbonic | 8.30 | 1 | 1 | 1 | 1 | 253 | 27.8 | 7.56 |
| | anhydrase-like protein | | | | | | | | |
| | OS = Medicago truncatula | | | | | | | | |
| | GN = MTR_8g096820 | | | | | | | | |
| | PE = 4 SV = 1 - | | | | | | | | |
| | [A0A072U593_MEDTR] | | | | | | | | |
| G7L8T3 | GDP dissociation inhibitor | 7.21 | 10 | 1 | 3 | 3 | 444 | 49.7 | 5.88 |
| | OS = Medicago truncatula | | | | | | | | |
| | GN = MTR_8g088620 | | | | | | | | |
| | PE = 2 SV = 1 - | | | | | | | | |
| | [G7L8T3_MEDTR] | | | | | | | | |
| A7Y7F0 | GDP-dissociation inhibitor | 6.53 | 18 | 1 | 3 | 3 | 444 | 49.7 | 5.95 |
| | OS = Lupinus albus PE = 2 | | | | | | | | |
| | SV = 1 - | | | | | | | | |
| | [A7Y7F0_LUPAL] | | | | | | | | |
| D4NXQ5 | Germin-like protein | 6.19 | 6 | 1 | 1 | 1 | 210 | 22.0 | 5.54 |
| | subfamily 2 member 2 | | | | | | | | |
| | (Fragment) OS = Arachis | | | | | | | | |
| | hypogaea GN = GLP6 | | | | | | | | |
| | PE = 2 SV = 1 - | | | | | | | | |
| | [D4NXQ5_ARAHY] | | | | | | | | |
| Q9ZP12 | Glucan | 5.74 | 1 | 1 | 1 | 1 | 331 | 35.6 | 5.71 |
| | endo-1,3-beta-d-glucosidase | | | | | | | | |
| | OS = Cicer arietinum | | | | | | | | |
| | PE = 2 SV = 1 - | | | | | | | | |
| | [Q9ZP12_CICAR] | | | | | | | | |
| A0A0B2P2K1 | Glucan | 4.52 | 10 | 1 | 1 | 1 | 310 | 34.1 | 6.21 |
| | endo-1,3-beta-glucosidase | | | | | | | | |
| | 1 OS = Glycine soja | | | | | | | | |
| | GN = glysoja_004511 PE = 3 | | | | | | | | |
| | SV = 1 - | | | | | | | | |
| | [A0A0B2P2K1_GLYSO] | | | | | | | | |
| A0A0B2Q7W6 | Glucose and ribitol | 6.83 | 5 | 1 | 2 | 4 | 293 | 31.8 | 7.36 |
| | dehydrogenase like 1 | | | | | | | | |
| | OS = Glycine soja | | | | | | | | |
| | GN = glysoja_012109 PE = 3 | | | | | | | | |
| | SV = 1 - | | | | | | | | |
| | [A0A0B2Q7W6_GLYSO] | | | | | | | | |
| V7B9B8 | Glucose-1-phosphate | 3.24 | 9 | 1 | 1 | 1 | 494 | 54.0 | 6.87 |
| | adenylyltransferase | | | | | | | | |
| | OS = Phaseolus vulgaris | | | | | | | | |
| | GN = PHAVU_008G284000g | | | | | | | | |
| | PE = 3 SV = 1 - | | | | | | | | |
| | [V7B9B8_PHAVU] | | | | | | | | |
| I3SRV1 | Glucose-6-phosphate | 3.70 | 15 | 2 | 2 | 2 | 514 | 58.7 | 6.28 |
| | 1-dehydrogenase | | | | | | | | |
| | OS = Lotus japonicus PE = 2 | | | | | | | | |
| | SV = 1 - [I3SRV1_LOTJA] | | | | | | | | |
| I1JTB9 | Glucose-6-phosphate | 1.64 | 12 | 1 | 1 | 1 | 487 | 53.6 | 6.14 |
| | isomerase OS = Glycine | | | | | | | | |
| | max PE = 3 SV = 1 - | | | | | | | | |
| | [I1JTB9_SOYBN] | | | | | | | | |
| A0A0B2NWV0 | Glutamate dehydrogenase | 2.91 | 4 | 1 | 1 | 1 | 412 | 44.7 | 6.40 |
| | OS = Glycine soja | | | | | | | | |
| | GN = glysoja_012937 PE = 3 | | | | | | | | |
| | SV = 1 - | | | | | | | | |
| | [A0A0B2NWV0_GLYSO] | | | | | | | | |
| B4UW78 | Glutamine synthetase | 7.01 | 1 | 1 | 1 | 1 | 157 | 17.3 | 5.12 |
| | GS56 (Fragment) | | | | | | | | |
| | OS = Arachis hypogaea | | | | | | | | |
| | PE = 2 SV = 1 - | | | | | | | | |
| | [B4UW78_ARAHY] | | | | | | | | |
| G7J5D8 | Glutaredoxin C4 | 9.09 | 1 | 1 | 1 | 1 | 121 | 13.0 | 6.77 |
| | OS = Medicago truncatula | | | | | | | | |
| | GN = MTR_3g077570 | | | | | | | | |
| | PE = 4 SV = 1 - | | | | | | | | |
| | [G7J5D8_MEDTR] | | | | | | | | |
| B4UW79 | Glutathione peroxidase | 22.22 | 8 | 5 | 5 | 5 | 216 | 24.2 | 9.50 |
| | (Fragment) OS = Arachis | | | | | | | | |
| | hypogaea PE = 2 SV = 1 - | | | | | | | | |
| | [B4UW79_ARAHY] | | | | | | | | |
| K7T3M0 | Glutathione S-transferase | 7.14 | 3 | 1 | 1 | 2 | 112 | 13.2 | 9.09 |
| | (Fragment) | | | | | | | | |
| | OS = Hedysarum pallidum | | | | | | | | |
| | GN = GST1 PE = 4 SV = 1 - | | | | | | | | |
| | [K7T3M0_9FABA] | | | | | | | | |
| B4UW81 | Glutathione S-transferase | 7.26 | 1 | 1 | 1 | 1 | 179 | 20.3 | 8.92 |
| | 2 OS = Arachis hypogaea | | | | | | | | |
| | PE = 2 SV = 1 - | | | | | | | | |
| | [B4UW81_ARAHY] | | | | | | | | |
| A0A0B2QDS3 | Glutathione S-transferase | 5.02 | 5 | 1 | 1 | 2 | 259 | 28.9 | 8.35 |
| | DHAR3, chloroplastic | | | | | | | | |
| | OS = Glycine soja | | | | | | | | |
| | GN = glysoja_017998 PE = 4 | | | | | | | | |
| | SV = 1 - | | | | | | | | |
| | [A0A0B2QDS3_GLYSO] | | | | | | | | |
| A0A0B2SEJ4 | Glutathione S-transferase | 15.19 | 14 | 2 | 2 | 2 | 158 | 18.8 | 8.32 |
| | F9 (Fragment) | | | | | | | | |
| | OS = Glycine soja | | | | | | | | |
| | GN = glysoja_041010 PE = 4 | | | | | | | | |
| | SV = 1 - | | | | | | | | |
| | [A0A0B2SEJ4_GLYSO] | | | | | | | | |
| Q9FZ11 | Gly1 OS = Arachis | 59.92 | 1 | 2 | 25 | 517 | 529 | 60.4 | 5.64 |
| | hypogaea GN = Gly1 PE = 2 | | | | | | | | |
| | SV = 1 - | | | | | | | | |
| | [Q9FZ11_ARAHY] | | | | | | | | |
| A0A0A6ZDT0 | Glyceraldehyde-3-phosphate | 42.47 | 30 | 2 | 7 | 32 | 186 | 20.1 | 8.32 |
| | dehydrogenase C2 | | | | | | | | |
| | OS = Arachis hypogaea | | | | | | | | |
| | PE = 3 SV = 1 - | | | | | | | | |
| | [A0A0A6ZDT0_ARAHY] | | | | | | | | |
| A0A0K0K9Q6 | Glyceraldehyde-3-phosphate | 7.66 | 3 | 1 | 3 | 3 | 496 | 53.0 | 7.37 |
| | dehydrogenase | | | | | | | | |
| | OS = Arachis diogoi PE = 2 | | | | | | | | |
| | SV = 1 - | | | | | | | | |
| | [A0A0K0K9Q6_9FABA] | | | | | | | | |
| I1KC66 | Glyceraldehyde-3-phosphate | 29.39 | 41 | 2 | 7 | 21 | 296 | 32.1 | 6.96 |
| | dehydrogenase | | | | | | | | |
| | OS = Glycine max PE = 3 | | | | | | | | |
| | SV = 1 - | | | | | | | | |
| | [I1KC66_SOYBN] | | | | | | | | |
| P34922 | Glyceraldehyde-3-phosphate | 26.63 | 48 | 2 | 8 | 25 | 338 | 36.6 | 7.05 |
| | dehydrogenase, | | | | | | | | |
| | cytosolic OS = Pisum | | | | | | | | |
| | sativum GN = GAPC1 | | | | | | | | |
| | PE = 2 SV = 1 - | | | | | | | | |
| | [G3PC_PEA] | | | | | | | | |
| O82580 | Glycinin (Fragment) | 44.77 | 1 | 4 | 17 | 335 | 507 | 58.3 | 5.94 |
| | OS = Arachis hypogaea | | | | | | | | |
| | GN = Arah3 PE = 2 SV = 1 - | | | | | | | | |
| | [O82580_ARAHY] | | | | | | | | |
| Q6IWG5 | Glycinin (Fragment) | 71.76 | 7 | 22 | 25 | 412 | 510 | 58.0 | 5.59 |
| | OS = Arachis hypogaea | | | | | | | | |
| | PE = 2 SV = 1 - | | | | | | | | |
| | [Q6IWG5_ARAHY] | | | | | | | | |
| A3KEY8 | Glycinin A3B4 subunit | 31.58 | 20 | 8 | 8 | 60 | 513 | 57.7 | 6.16 |
| | OS = Glycine soja | | | | | | | | |
| | GN = A3B4 PE = 2 SV = 1 - | | | | | | | | |
| | [A3KEY8_GLYSO] | | | | | | | | |
| A0A0B2PSZ5 | Glycinin G1 OS = Glycine | 40.93 | 4 | 6 | 10 | 24 | 496 | 55.8 | 6.28 |
| | soja GN = glysoja_019175 | | | | | | | | |
| | PE = 4 SV = 1 - | | | | | | | | |
| | [A0A0B2PSZ5_GLYSO] | | | | | | | | |
| P04405 | Glycinin G2 OS = Glycine | 44.54 | 3 | 6 | 11 | 35 | 485 | 54.4 | 5.58 |
| | max GN = Gy2 PE = 1 SV = 2 - | | | | | | | | |
| | [GLYG2_SOYBN] | | | | | | | | |
| A0A0B2P4D2 | Glycinin G3 OS = Glycine | 26.20 | 4 | 3 | 6 | 13 | 481 | 54.1 | 5.97 |
| | soja GN = glysoja_002527 | | | | | | | | |
| | PE = 4 SV = 1 - | | | | | | | | |
| | [A0A0B2P4D2_GLYSO] | | | | | | | | |
| Q9SQH7 | Glycinin OS = Arachis | 38.11 | 1 | 1 | 12 | 336 | 530 | 61.0 | 5.68 |
| | hypogaea GN = Arah4 | | | | | | | | |
| | PE = 2 SV = 1 - | | | | | | | | |
| | [Q9SQH7_ARAHY] | | | | | | | | |
| G7KXJ4 | Glycoside hydrolase | 3.13 | 1 | 1 | 1 | 1 | 288 | 33.3 | 7.14 |
| | family 81 protein | | | | | | | | |
| | OS = Medicago truncatula | | | | | | | | |
| | GN = MTR_7g112730 | | | | | | | | |
| | PE = 4 SV = 1 - | | | | | | | | |
| | [G7KXJ4_MEDTR] | | | | | | | | |
| A0A072USM8 | Glycyl-tRNA | 2.81 | 9 | 2 | 2 | 2 | 640 | 72.2 | 6.23 |
| | synthetase/glycine-tRNA | | | | | | | | |
| | ligase OS = Medicago | | | | | | | | |
| | truncatula | | | | | | | | |
| | GN = MTR_3g006730 | | | | | | | | |
| | PE = 3 SV = 1 - | | | | | | | | |
| | [A0A072USM8_MEDTR] | | | | | | | | |
| Q1RSH3 | GroEL-like chaperone, | 17.94 | 4 | 1 | 8 | 8 | 574 | 61.1 | 6.09 |
| | ATPase OS = Medicago | | | | | | | | |
| | truncatula | | | | | | | | |
| | GN = MTR_1g090140 | | | | | | | | |
| | PE = 3 SV = 1 - | | | | | | | | |
| | [Q1RSH3_MEDTR] | | | | | | | | |
| B4UW54 | GroES-like protein | 5.45 | 1 | 1 | 1 | 1 | 202 | 21.2 | 8.78 |
| | (Fragment) OS = Arachis | | | | | | | | |
| | hypogaea PE = 2 SV = 1 - | | | | | | | | |
| | [B4UW54_ARAHY] | | | | | | | | |
| E3NYT1 | Harpin binding protein | 7.27 | 10 | 1 | 1 | 1 | 165 | 18.0 | 7.27 |
| | (Fragment) OS = Arachis | | | | | | | | |
| | diogoi PE = 2 SV = 1 - | | | | | | | | |
| | [E3NYT1_9FABA] | | | | | | | | |
| G7JFG6 | Heat shock 70 kDa protein | 1.30 | 1 | 1 | 1 | 1 | 692 | 77.1 | 5.26 |
| | OS = Medicago truncatula | | | | | | | | |
| | GN = MTR_4g115780 | | | | | | | | |
| | PE = 3 SV = 1 - | | | | | | | | |
| | [G7JFG6_MEDTR] | | | | | | | | |
| G7JFK1 | Heat shock 70 kDa protein | 22.05 | 52 | 0 | 11 | 16 | 653 | 71.5 | 5.39 |
| | OS = Medicago truncatula | | | | | | | | |
| | GN = MTR_4g130540 | | | | | | | | |
| | PE = 3 SV = 1 - | | | | | | | | |
| | [G7JFK1_MEDTR] | | | | | | | | |
| G7L003 | Heat shock 70 kDa protein | 7.00 | 13 | 1 | 3 | 5 | 614 | 68.3 | 6.06 |
| | OS = Medicago truncatula | | | | | | | | |
| | GN = MTR_7g026090 | | | | | | | | |
| | PE = 3 SV = 2 - | | | | | | | | |
| | [G7L003_MEDTR] | | | | | | | | |
| A0A072TUS4 | Heat shock 70 kDa protein | 17.22 | 24 | 1 | 9 | 11 | 668 | 73.7 | 5.19 |
| | OS = Medicago truncatula | | | | | | | | |
| | GN = MTR_8g099795 | | | | | | | | |
| | PE = 3 SV = 1 - | | | | | | | | |
| | [A0A072TUS4_MEDTR] | | | | | | | | |
| Q01899 | Heat shock 70 kDa | 16.74 | 12 | 3 | 9 | 10 | 675 | 72.5 | 6.20 |
| | protein, mitochondrial | | | | | | | | |
| | OS = Phaseolus vulgaris | | | | | | | | |
| | PE = 2 SV = 1 - | | | | | | | | |
| | [HSP7M_PHAVU] | | | | | | | | |
| A0A072UM13 | Heat shock cognate 70 | 25.58 | 91 | 2 | 14 | 21 | 649 | 71.0 | 5.15 |
| | kDa protein OS = Medicago | | | | | | | | |
| | truncatula | | | | | | | | |
| | GN = MTR_4g063720 | | | | | | | | |
| | PE = 3 SV = 1 - | | | | | | | | |
| | [A0A072UM13_MEDTR] | | | | | | | | |
| Q2HT97 | Heat shock cognate 70 | 23.11 | 80 | 1 | 13 | 20 | 649 | 71.0 | 5.22 |
| | kDa protein OS = Medicago | | | | | | | | |
| | truncatula | | | | | | | | |
| | GN = MTR_7g099680 | | | | | | | | |
| | PE = 3 SV = 1 - | | | | | | | | |
| | [Q2HT97_MEDTR] | | | | | | | | |
| A0A072TV12 | Heat shock cognate 70 | 18.98 | 24 | 1 | 10 | 11 | 669 | 73.6 | 5.12 |
| | kDa-like protein | | | | | | | | |
| | OS = Medicago truncatula | | | | | | | | |
| | GN = MTR_8g099945 | | | | | | | | |
| | PE = 3 SV = 1 - | | | | | | | | |
| | [A0A072TV12_MEDTR] | | | | | | | | |
| A0A0B2PDE3 | Heat shock cognate protein | 11.65 | 1 | 1 | 1 | 1 | 103 | 11.3 | 8.41 |
| | 80 (Fragment) | | | | | | | | |
| | OS = Glycine soja | | | | | | | | |
| | GN = glysoja_048590 PE = 4 | | | | | | | | |
| | SV = 1 - | | | | | | | | |
| | [A0A0B2PDE3_GLYSO] | | | | | | | | |
| B4UW89 | Heat shock protein 2 | 35.63 | 13 | 4 | 5 | 6 | 160 | 18.1 | 5.71 |
| | OS = Arachis hypogaea | | | | | | | | |
| | PE = 2 SV = 1 - | | | | | | | | |
| | [B4UW89_ARAHY] | | | | | | | | |
| G7I7Q4 | Heat shock protein 81-2 | 10.88 | 23 | 1 | 8 | 8 | 818 | 94.1 | 4.89 |
| | OS = Medicago truncatula | | | | | | | | |
| | GN = MTR_1g025430 | | | | | | | | |
| | PE = 3 SV = 1 - | | | | | | | | |
| | [G7I7Q4_MEDTR] | | | | | | | | |
| G7IDZ4 | Heat shock protein 81-2 | 11.47 | 14 | 0 | 7 | 9 | 689 | 79.3 | 5.05 |
| | OS = Medicago truncatula | | | | | | | | |
| | GN = MTR_1g099840 | | | | | | | | |
| | PE = 3 SV = 1 - | | | | | | | | |
| | [G7IDZ4_MEDTR] | | | | | | | | |
| G7K4R2 | Heat shock protein 81-2 | 11.73 | 13 | 0 | 7 | 8 | 699 | 80.1 | 5.03 |
| | OS = Medicago truncatula | | | | | | | | |
| | GN = MTR_5g096430 | | | | | | | | |
| | PE = 3 SV = 1 - | | | | | | | | |
| | [G7K4R2_MEDTR] | | | | | | | | |
| Q40980 | Heat shock protein hsp70 | 20.37 | 53 | 0 | 11 | 16 | 648 | 71.1 | 5.26 |
| | OS = Pisum sativum | | | | | | | | |
| | GN = hsp70 PE = 2 SV = 1 - | | | | | | | | |
| | [Q40980_PEA] | | | | | | | | |
| A7Y7E4 | Heat shock protein | 7.98 | 6 | 1 | 2 | 2 | 213 | 24.5 | 7.20 |
| | OS = Stylosanthes hamata | | | | | | | | |
| | PE = 2 SV = 1 - | | | | | | | | |
| | [A7Y7E4_STYHA] | | | | | | | | |
| T2DML9 | Histone H1 subtype 5 | 4.86 | 6 | 2 | 2 | 2 | 288 | 30.4 | 10.64 |
| | OS = Phaseolus vulgaris | | | | | | | | |
| | PE = 2 SV = 1 - | | | | | | | | |
| | [T2DML9_PHAVU] | | | | | | | | |
| C6T2B0 | Histone H2A OS = Glycine | 12.50 | 56 | 1 | 2 | 2 | 128 | 13.7 | 10.36 |
| | max PE = 2 SV = 1 - | | | | | | | | |
| | [C6T2B0_SOYBN] | | | | | | | | |
| V7BPQ3 | Histone H2A | 11.68 | 60 | 1 | 2 | 3 | 137 | 14.6 | 10.62 |
| | OS = Phaseolus vulgaris | | | | | | | | |
| | GN = PHAVU_006G090400g | | | | | | | | |
| | PE = 3 SV = 1 - | | | | | | | | |
| | [V7BPQ3_PHAVU] | | | | | | | | |
| I3SLS9 | Histone H2B OS = Lotus | 31.43 | 56 | 3 | 3 | 3 | 105 | 11.8 | 10.17 |
| | japonicus PE = 2 SV = 1 - | | | | | | | | |
| | [I3SLS9_LOTJA] | | | | | | | | |
| A0A0B2P491 | Histone H4 OS = Glycine | 50.49 | 21 | 5 | 5 | 7 | 103 | 11.4 | 11.33 |
| | soja GN = glysoja_002448 | | | | | | | | |
| | PE = 3 SV = 1 - | | | | | | | | |
| | [A0A0B2P491_GLYSO] | | | | | | | | |
| A0A072TQP2 | Homogentisate | 3.99 | 1 | 1 | 1 | 1 | 451 | 50.2 | 6.16 |
| | 1,2-dioxygenase | | | | | | | | |
| | OS = Medicago truncatula | | | | | | | | |
| | GN = MTR_8g463280 | | | | | | | | |
| | PE = 4 SV = 1 - | | | | | | | | |
| | [A0A072TQP2_MEDTR] | | | | | | | | |
| G7IQ96 | HSPA4 nucleotide-binding | 2.57 | 3 | 1 | 4 | 4 | 1558 | 174.9 | 5.95 |
| | domain protein | | | | | | | | |
| | OS = Medicago truncatula | | | | | | | | |
| | GN = MTR_2g040220 | | | | | | | | |
| | PE = 3 SV = 1 - | | | | | | | | |
| | [G7IQ96_MEDTR] | | | | | | | | |
| A0A072UG39 | Hydrolyzing O-glycosyl | 2.10 | 2 | 1 | 1 | 1 | 523 | 59.5 | 6.33 |
| | compounds hydrolase | | | | | | | | |
| | OS = Medicago truncatula | | | | | | | | |
| | GN = MTR_5g488355 | | | | | | | | |
| | PE = 3 SV = 1 - | | | | | | | | |
| | [A0A072UG39_MEDTR] | | | | | | | | |
| P24337 | Hydrophobic seed protein | 13.75 | 4 | 1 | 1 | 1 | 80 | 8.4 | 6.28 |
| | OS = Glycine max PE = 1 | | | | | | | | |
| | SV = 1 - [HPSE_SOYBN] | | | | | | | | |
| Q45NN8 | Hydroxyacylglutathione | 5.85 | 5 | 1 | 1 | 1 | 171 | 18.6 | 5.85 |
| | hydrolase (Fragment) | | | | | | | | |
| | OS = Medicago sativa PE = 2 | | | | | | | | |
| | SV = 1 - | | | | | | | | |
| | [Q45NN8_MEDSA] | | | | | | | | |
| A0A0B2QVY6 | Hydroxyacylglutathione | 9.02 | 5 | 1 | 1 | 1 | 133 | 14.1 | 6.39 |
| | hydrolase cytoplasmic | | | | | | | | |
| | OS = Glycine soja | | | | | | | | |
| | GN = glysoja_038287 PE = 4 | | | | | | | | |
| | SV = 1 - | | | | | | | | |
| | [A0A0B2QVY6_GLYSO] | | | | | | | | |
| A0A072U0M2 | ICE-like protease | 2.91 | 7 | 1 | 1 | 1 | 412 | 44.9 | 5.11 |
| | (Caspase) p20 domain | | | | | | | | |
| | protein OS = Medicago | | | | | | | | |
| | truncatula | | | | | | | | |
| | GN = MTR_8g447080 | | | | | | | | |
| | PE = 4 SV = 1 - | | | | | | | | |
| | [A0A072U0M2_MEDTR] | | | | | | | | |
| A0A0B2NS29 | Importin subunit beta-1 | 1.03 | 2 | 1 | 1 | 1 | 870 | 96.3 | 4.78 |
| | OS = Glycine soja | | | | | | | | |
| | GN = glysoja_017572 PE = 4 | | | | | | | | |
| | SV = 1 - | | | | | | | | |
| | [A0A0B2NS29_GLYSO] | | | | | | | | |
| G7IT36 | Isocitrate dehydrogenase | 7.45 | 32 | 3 | 3 | 3 | 416 | 46.7 | 7.99 |
| | [NADP] OS = Medicago | | | | | | | | |
| | truncatula | | | | | | | | |
| | GN = MTR_2g062840 | | | | | | | | |
| | PE = 2 SV = 1 - | | | | | | | | |
| | [G7IT36_MEDTR] | | | | | | | | |
| A0A0B2R2L5 | Ketol-acid | 4.35 | 10 | 1 | 2 | 2 | 529 | 58.2 | 7.27 |
| | reductoisomerase, | | | | | | | | |
| | chloroplastic OS = Glycine | | | | | | | | |
| | soja GN = glysoja_007667 | | | | | | | | |
| | PE = 4 SV = 1 - | | | | | | | | |
| | [A0A0B2R2L5_GLYSO] | | | | | | | | |
| O82043 | Ketol-acid | 4.13 | 6 | 1 | 2 | 2 | 581 | 62.8 | 7.08 |
| | reductoisomerase, | | | | | | | | |
| | chloroplastic OS = Pisum | | | | | | | | |
| | sativum GN = PGAAIR | | | | | | | | |
| | PE = 2 SV = 1 - [ILV5_PEA] | | | | | | | | |
| B4UWB2 | Kunitz trypsin inhibitor 4 | 10.34 | 1 | 2 | 2 | 2 | 203 | 21.6 | 8.79 |
| | OS = Arachis hypogaea | | | | | | | | |
| | PE = 2 SV = 1 - | | | | | | | | |
| | [B4UWB2_ARAHY] | | | | | | | | |
| A0A0B2NXT3 | Kunitz-type trypsin | 14.29 | 2 | 2 | 2 | 2 | 203 | 22.6 | 7.24 |
| | inhibitor KTI1 | | | | | | | | |
| | OS = Glycine soja | | | | | | | | |
| | GN = glysoja_028524 PE = 4 | | | | | | | | |
| | SV = 1 - | | | | | | | | |
| | [A0A0B2NXT3_GLYSO] | | | | | | | | |
| A0A0B2RIW3 | Kunitz-type trypsin | 6.88 | 4 | 1 | 1 | 1 | 160 | 17.7 | 5.36 |
| | inhibitor KTI1 | | | | | | | | |
| | OS = Glycine soja | | | | | | | | |
| | GN = glysoja_044930 PE = 4 | | | | | | | | |
| | SV = 1 - | | | | | | | | |
| | [A0A0B2RIW3_GLYSO] | | | | | | | | |
| B4UWA1 | Lactoylglutathione lyase | 33.67 | 1 | 4 | 6 | 6 | 196 | 22.3 | 8.32 |
| | (Fragment) OS = Arachis | | | | | | | | |
| | hypogaea PE = 2 SV = 1 - | | | | | | | | |
| | [B4UWA1_ARAHY] | | | | | | | | |
| B4UWB4 | Lactoylglutathione lyase | 22.50 | 1 | 2 | 2 | 2 | 200 | 22.9 | 5.15 |
| | (Fragment) OS = Arachis | | | | | | | | |
| | hypogaea PE = 2 SV = 1 - | | | | | | | | |
| | [B4UWB4_ARAHY] | | | | | | | | |
| I3S2W4 | Lactoylglutathione lyase | 14.60 | 9 | 1 | 4 | 5 | 274 | 30.6 | 5.78 |
| | OS = Medicago truncatula | | | | | | | | |
| | PE = 2 SV = 1 - | | | | | | | | |
| | [I3S2W4_MEDTR] | | | | | | | | |
| G7JJ88 | Lactoylglutathione | 7.83 | 7 | 1 | 2 | 3 | 281 | 31.5 | 5.26 |
| | lyase-like protein | | | | | | | | |
| | OS = Medicago truncatula | | | | | | | | |
| | GN = MTR_4g132270 | | | | | | | | |
| | PE = 4 SV = 2 - | | | | | | | | |
| | [G7JJ88_MEDTR] | | | | | | | | |
| E5FHY1 | Late embryogenesis | 62.77 | 9 | 3 | 6 | 23 | 94 | 10.1 | 6.13 |
| | abundant protein group 1 | | | | | | | | |
| | protein OS = Arachis | | | | | | | | |
| | hypogaea GN = LEA1-2 | | | | | | | | |
| | PE = 2 SV = 1 - | | | | | | | | |
| | [E5FHY1_ARAHY] | | | | | | | | |
| E5FHY3 | Late embryogenesis | 17.39 | 1 | 3 | 2 | 8 | 207 | 21.0 | 7.84 |
| | abundant protein group 2 | | | | | | | | |
| | protein dehydrin | | | | | | | | |
| | OS = Arachis hypogaea | | | | | | | | |
| | GN = DHN1 PE = 2 SV = 1 - | | | | | | | | |
| | [E5FHY3_ARAHY] | | | | | | | | |
| E5FHY6 | Late embryogenesis | 28.83 | 1 | 5 | 5 | 7 | 163 | 18.0 | 9.70 |
| | abundant protein group 3 | | | | | | | | |
| | protein (Fragment) | | | | | | | | |
| | OS = Arachis hypogaea | | | | | | | | |
| | GN = LEA3-3 PE = 2 SV = 1 - | | | | | | | | |
| | [E5FHY6_ARAHY] | | | | | | | | |
| E5FHY4 | Late embryogenesis | 51.30 | 1 | 7 | 7 | 10 | 154 | 16.5 | 8.41 |
| | abundant protein group 3 | | | | | | | | |
| | protein OS = Arachis | | | | | | | | |
| | hypogaea GN = LEA3-1 | | | | | | | | |
| | PE = 2 SV = 1 - | | | | | | | | |
| | [E5FHY4_ARAHY] | | | | | | | | |
| E5FHY7 | Late embryogenesis | 17.50 | 1 | 4 | 4 | 6 | 200 | 22.5 | 6.81 |
| | abundant protein group 3 | | | | | | | | |
| | protein OS = Arachis | | | | | | | | |
| | hypogaea GN = LEA3-5 | | | | | | | | |
| | PE = 2 SV = 1 - | | | | | | | | |
| | [E5FHY7_ARAHY] | | | | | | | | |
| E5FHY8 | Late embryogenesis | 45.45 | 2 | 4 | 4 | 6 | 66 | 7.0 | 9.41 |
| | abundant protein group 3 | | | | | | | | |
| | protein OS = Arachis | | | | | | | | |
| | hypogaea GN = LEA3-7 | | | | | | | | |
| | PE = 2 SV = 1 - | | | | | | | | |
| | [E5FHY8_ARAHY] | | | | | | | | |
| E5FHY9 | Late embryogenesis | 13.37 | 3 | 2 | 2 | 6 | 172 | 17.9 | 9.47 |
| | abundant protein group 4 | | | | | | | | |
| | protein OS = Arachis | | | | | | | | |
| | hypogaea GN = LEA4-1 | | | | | | | | |
| | PE = 2 SV = 1 - | | | | | | | | |
| | [E5FHY9_ARAHY] | | | | | | | | |
| E5FHZ2 | Late embryogenesis | 43.02 | 6 | 8 | 8 | 14 | 265 | 27.4 | 4.87 |
| | abundant protein group 5 | | | | | | | | |
| | protein OS = Arachis | | | | | | | | |
| | hypogaea GN = LEA5-2 | | | | | | | | |
| | PE = 2 SV = 1 - | | | | | | | | |
| | [E5FHZ2_ARAHY] | | | | | | | | |
| E5FHZ5 | Late embryogenesis | 18.71 | 1 | 1 | 3 | 3 | 155 | 17.0 | 5.91 |
| | abundant protein group 7 | | | | | | | | |
| | protein OS = Arachis | | | | | | | | |
| | hypogaea GN = LEA7-1 | | | | | | | | |
| | PE = 2 SV = 1 - | | | | | | | | |
| | [E5FHZ5_ARAHY] | | | | | | | | |
| E5FHZ7 | Late embryogenesis | 19.34 | 2 | 3 | 3 | 4 | 181 | 19.4 | 9.99 |
| | abundant protein group 9 | | | | | | | | |
| | protein OS = Arachis | | | | | | | | |
| | hypogaea GN = LEA9-1 | | | | | | | | |
| | PE = 2 SV = 1 - | | | | | | | | |
| | [E5FHZ7_ARAHY] | | | | | | | | |
| C6TLT7 | Late-embryogenesis | 9.69 | 12 | 1 | 3 | 3 | 320 | 35.6 | 4.98 |
| | abundant protein 1 | | | | | | | | |
| | OS = Glycine max | | | | | | | | |
| | GN = LEA-1 PE = 2 SV = 1 - | | | | | | | | |
| | [C6TLT7_SOYBN] | | | | | | | | |
| Q4U4M1 | LEA protein (Fragment) | 52.63 | 15 | 1 | 4 | 16 | 95 | 10.4 | 5.31 |
| | OS = Arachis hypogaea | | | | | | | | |
| | PE = 2 SV = 1 - | | | | | | | | |
| | [Q4U4M1_ARAHY] | | | | | | | | |
| Q45W84 | Lea protein 2 OS = Arachis | 7.14 | 1 | 1 | 1 | 1 | 126 | 13.7 | 9.39 |
| | hypogaea PE = 2 SV = 1 - | | | | | | | | |
| | [Q45W84_ARAHY] | | | | | | | | |
| Q53I54 | Legumin-like protein | 2.34 | 1 | 1 | 1 | 4 | 512 | 58.4 | 5.78 |
| | OS = Lupinus albus PE = 2 | | | | | | | | |
| | SV = 2 - [Q53I54_LUPAL] | | | | | | | | |
| A0A0B2SEG7 | Leucine--tRNA ligase, | 1.28 | 4 | 1 | 1 | 1 | 860 | 97.0 | 6.38 |
| | cytoplasmic OS = Glycine | | | | | | | | |
| | soja GN = glysoja_018930 | | | | | | | | |
| | PE = 4 SV = 1 - | | | | | | | | |
| | [A0A0B2SEG7_GLYSO] | | | | | | | | |
| A0A072VMF0 | Linker histone H1 and h5 | 6.25 | 11 | 2 | 2 | 2 | 224 | 23.3 | 11.09 |
| | family protein | | | | | | | | |
| | OS = Medicago truncatula | | | | | | | | |
| | GN = MTR_1g070440 | | | | | | | | |
| | PE = 3 SV = 1 - | | | | | | | | |
| | [A0A072VMF0_MEDTR] | | | | | | | | |
| A0A0B2P197 | Linoleate 9S-lipoxygenase | 4.64 | 3 | 1 | 2 | 2 | 453 | 51.5 | 6.15 |
| | 1 OS = Glycine soja | | | | | | | | |
| | GN = glysoja_009579 PE = 4 | | | | | | | | |
| | SV = 1 - | | | | | | | | |
| | [A0A0B2P197_GLYSO] | | | | | | | | |
| A0A0C4G635 | Lipoxygenase (Fragment) | 3.87 | 3 | 1 | 2 | 2 | 724 | 82.1 | 6.15 |
| | OS = Phaseolus coccineus | | | | | | | | |
| | GN = LOXC PE = 2 SV = 1 - | | | | | | | | |
| | [A0A0C4G635_PHACN] | | | | | | | | |
| B4UWB9 | Lipoxygenase 1 | 20.08 | 1 | 3 | 3 | 5 | 239 | 26.7 | 8.76 |
| | (Fragment) OS = Arachis | | | | | | | | |
| | hypogaea PE = 2 SV = 1 - | | | | | | | | |
| | [B4UWB9_ARAHY] | | | | | | | | |
| B4UWC0 | Lipoxygenase 2 | 31.84 | 1 | 1 | 5 | 9 | 179 | 20.5 | 5.21 |
| | (Fragment) OS = Arachis | | | | | | | | |
| | hypogaea PE = 2 SV = 1 - | | | | | | | | |
| | [B4UWC0_ARAHY] | | | | | | | | |
| Q4JME6 | Lipoxygenase OS = Arachis | 42.87 | 25 | 17 | 28 | 67 | 863 | 97.4 | 5.53 |
| | hypogaea GN = Pnlox3 | | | | | | | | |
| | PE = 2 SV = 1 - | | | | | | | | |
| | [Q4JME6_ARAHY] | | | | | | | | |
| I1M597 | Lipoxygenase OS = Glycine | 23.08 | 6 | 6 | 9 | 20 | 702 | 78.7 | 6.44 |
| | max PE = 3 SV = 2 - | | | | | | | | |
| | [I1M597_SOYBN] | | | | | | | | |
| A0A0B2RDZ1 | Lipoxygenase OS = Glycine | 8.80 | 25 | 1 | 6 | 18 | 841 | 94.7 | 6.44 |
| | soja GN = glysoja_010450 | | | | | | | | |
| | PE = 3 SV = 1 - | | | | | | | | |
| | [A0A0B2RDZ1_GLYSO] | | | | | | | | |
| A0A0B2RHI0 | Lipoxygenase OS = Glycine | 15.99 | 27 | 4 | 10 | 20 | 857 | 96.7 | 6.58 |
| | soja GN = glysoja_022392 | | | | | | | | |
| | PE = 3 SV = 1 - | | | | | | | | |
| | [A0A0B2RHI0_GLYSO] | | | | | | | | |
| A0A0B2PUU0 | Lipoxygenase OS = Glycine | 7.49 | 13 | 1 | 5 | 8 | 868 | 97.1 | 7.40 |
| | soja GN = glysoja_023321 | | | | | | | | |
| | PE = 3 SV = 1 - | | | | | | | | |
| | [A0A0B2PUU0_GLYSO] | | | | | | | | |
| G7IS30 | Lipoxygenase | 8.83 | 21 | 0 | 8 | 18 | 861 | 97.3 | 6.54 |
| | OS = Medicago truncatula | | | | | | | | |
| | GN = MTR_2g099570 | | | | | | | | |
| | PE = 3 SV = 1 - | | | | | | | | |
| | [G7IS30_MEDTR] | | | | | | | | |
| G7LI99 | Lipoxygenase | 6.19 | 7 | 1 | 5 | 7 | 856 | 96.1 | 5.50 |
| | OS = Medicago truncatula | | | | | | | | |
| | GN = MTR_8g018420 | | | | | | | | |
| | PE = 3 SV = 1 - | | | | | | | | |
| | [G7LI99_MEDTR] | | | | | | | | |
| G7L7J9 | Lipoxygenase | 2.22 | 2 | 1 | 1 | 1 | 676 | 76.4 | 9.07 |
| | OS = Medicago truncatula | | | | | | | | |
| | GN = MTR_8g020970 | | | | | | | | |
| | PE = 3 SV = 1 - | | | | | | | | |
| | [G7L7J9_MEDTR] | | | | | | | | |
| V7C680 | Lipoxygenase | 1.59 | 1 | 1 | 1 | 1 | 882 | 100.2 | 6.61 |
| | OS = Phaseolus vulgaris | | | | | | | | |
| | GN = PHAVU_004G168400g | | | | | | | | |
| | PE = 3 SV = 1 - | | | | | | | | |
| | [V7C680_PHAVU] | | | | | | | | |
| G7JZY6 | M20 peptidase | 2.96 | 1 | 1 | 1 | 1 | 439 | 48.2 | 5.48 |
| | acetylornithine deacetylase | | | | | | | | |
| | OS = Medicago truncatula | | | | | | | | |
| | GN = MTR_5g014410 | | | | | | | | |
| | PE = 4 SV = 1 - | | | | | | | | |
| | [G7JZY6_MEDTR] | | | | | | | | |
| G7JB63 | Major intrinsic protein | 4.65 | 29 | 1 | 1 | 1 | 215 | 23.0 | 8.13 |
| | (MIP) family transporter | | | | | | | | |
| | OS = Medicago truncatula | | | | | | | | |
| | GN = MTR_3g070210 | | | | | | | | |
| | PE = 3 SV = 1 - | | | | | | | | |
| | [G7JB63_MEDTR] | | | | | | | | |
| T2B995 | Malate dehydrogenase | 10.67 | 17 | 1 | 2 | 2 | 356 | 37.5 | 7.39 |
| | OS = Arachis hypogaea | | | | | | | | |
| | GN = MDH PE = 2 SV = 1 - | | | | | | | | |
| | [T2B995_ARAHY] | | | | | | | | |
| I1MTU1 | Malate dehydrogenase | 13.57 | 10 | 1 | 4 | 8 | 361 | 38.3 | 6.27 |
| | OS = Glycine max PE = 3 | | | | | | | | |
| | SV = 1 - | | | | | | | | |
| | [I1MTU1_SOYBN] | | | | | | | | |
| A0A0B2PZC5 | Malate dehydrogenase | 13.03 | 23 | 1 | 3 | 5 | 353 | 37.4 | 7.64 |
| | OS = Glycine soja | | | | | | | | |
| | GN = glysoja_027322 PE = 3 | | | | | | | | |
| | SV = 1 - | | | | | | | | |
| | [A0A0B2PZC5_GLYSO] | | | | | | | | |
| F5B9G0 | Malate dehydrogenase | 13.86 | 28 | 4 | 4 | 4 | 332 | 35.5 | 6.32 |
| | OS = Lupinus angustifolius | | | | | | | | |
| | GN = MDH1 PE = 2 SV = 1 - | | | | | | | | |
| | [F5B9G0_LUPAN] | | | | | | | | |
| A0A0A1G6F9 | Malate dehydrogenase | 42.32 | 14 | 7 | 9 | 16 | 345 | 36.1 | 8.56 |
| | OS = Stylosanthes | | | | | | | | |
| | guianensis GN = MDH1 | | | | | | | | |
| | PE = 2 SV = 1 - | | | | | | | | |
| | [A0A0A1G6F9_9FABA] | | | | | | | | |
| A0A0B2S8Y3 | Malic enzyme | 5.58 | 37 | 1 | 2 | 2 | 591 | 65.4 | 6.81 |
| | OS = Glycine soja | | | | | | | | |
| | GN = glysoja_036409 PE = 3 | | | | | | | | |
| | SV = 1 - | | | | | | | | |
| | [A0A0B2S8Y3_GLYSO] | | | | | | | | |
| Q8H1L8 | Manganese superoxide | 14.00 | 6 | 1 | 1 | 2 | 100 | 11.0 | 7.72 |
| | dismutase (Fragment) | | | | | | | | |
| | OS = Medicago sativa | | | | | | | | |
| | GN = MnSOD PE = 2 SV = 1 - | | | | | | | | |
| | [Q8H1L8_MEDSA] | | | | | | | | |
| B4UWC9 | MCT-1 protein-like protein | 10.24 | 1 | 1 | 1 | 1 | 127 | 14.2 | 8.46 |
| | (Fragment) OS = Arachis | | | | | | | | |
| | hypogaea PE = 2 SV = 1 - | | | | | | | | |
| | [B4UWC9_ARAHY] | | | | | | | | |
| A0A0B2RHP8 | Membrane steroid-binding | 4.78 | 10 | 1 | 1 | 1 | 209 | 22.7 | 4.84 |
| | protein 2 OS = Glycine soja | | | | | | | | |
| | GN = glysoja_029638 PE = 4 | | | | | | | | |
| | SV = 1 - | | | | | | | | |
| | [A0A0B2RHP8_GLYSO] | | | | | | | | |
| Q71EW8 | Methionine synthase | 15.20 | 18 | 1 | 10 | 16 | 763 | 84.2 | 6.33 |
| | OS = Glycine max PE = 2 | | | | | | | | |
| | SV = 1 - | | | | | | | | |
| | [Q71EW8_SOYBN] | | | | | | | | |
| G7JK45 | Monocopper oxidase | 2.54 | 1 | 1 | 1 | 1 | 590 | 66.2 | 8.91 |
| | SKS1-like protein | | | | | | | | |
| | OS = Medicago truncatula | | | | | | | | |
| | GN = MTR_4g101650 | | | | | | | | |
| | PE = 4 SV = 1 - | | | | | | | | |
| | [G7JK45_MEDTR] | | | | | | | | |
| A0A0K1LXL0 | Monodehydroascorbate | 4.02 | 3 | 1 | 1 | 1 | 224 | 24.3 | 5.48 |
| | reductase (Fragment) | | | | | | | | |
| | OS = Trifolium repens | | | | | | | | |
| | GN = MR PE = 4 SV = 1 - | | | | | | | | |
| | [A0A0K1LXL0_TRIRP] | | | | | | | | |
| B4UWD2 | Myo-inisitol oxygenase | 16.81 | 1 | 1 | 2 | 2 | 119 | 14.1 | 4.60 |
| | (Fragment) OS = Arachis | | | | | | | | |
| | hypogaea PE = 2 SV = 1 - | | | | | | | | |
| | [B4UWD2_ARAHY] | | | | | | | | |
| A0A072TN27 | Myo-inositol oxygenase | 7.78 | 5 | 1 | 2 | 2 | 270 | 31.5 | 5.11 |
| | OS = Medicago truncatula | | | | | | | | |
| | GN = MTR_8g015220 | | | | | | | | |
| | PE = 4 SV = 1 - | | | | | | | | |
| | [A0A072TN27_MEDTR] | | | | | | | | |
| A0A0B2QWY2 | NADH dehydrogenase | 2.44 | 5 | 1 | 1 | 1 | 657 | 71.7 | 6.35 |
| | [ubiquinone] iron-sulfur | | | | | | | | |
| | protein 1, mitochondrial | | | | | | | | |
| | OS = Glycine soja | | | | | | | | |
| | GN = glysoja_034805 PE = 3 | | | | | | | | |
| | SV = 1 - | | | | | | | | |
| | [A0A0B2QWY2_GLYSO] | | | | | | | | |
| E9KZM7 | NADH dehydrogenase | 6.84 | 12 | 1 | 1 | 1 | 190 | 22.6 | 7.97 |
| | subunit 9 OS = Vigna | | | | | | | | |
| | radiata GN = nad9 PE = 3 | | | | | | | | |
| | SV = 1 - | | | | | | | | |
| | [E9KZM7_VIGRA] | | | | | | | | |
| Q8GSP3 | Nascent | 19.01 | 19 | 2 | 2 | 2 | 142 | 15.4 | 5.67 |
| | polypeptide-associated | | | | | | | | |
| | complex subunit beta | | | | | | | | |
| | OS = Lotus japonicus PE = 3 | | | | | | | | |
| | SV = 1 - | | | | | | | | |
| | [Q8GSP3_LOTJA] | | | | | | | | |
| A0A0B2RKL6 | N-carbamoylputrescine | 6.02 | 10 | 1 | 1 | 1 | 299 | 33.5 | 6.09 |
| | amidase OS = Glycine soja | | | | | | | | |
| | GN = glysoja_038798 PE = 4 | | | | | | | | |
| | SV = 1 - | | | | | | | | |
| | [A0A0B2RKL6_GLYSO] | | | | | | | | |
| O81609 | Nodule-enhanced malate | 8.79 | 3 | 1 | 3 | 3 | 398 | 41.8 | 7.75 |
| | dehydrogenase OS = Pisum | | | | | | | | |
| | sativum GN = nemdh PE = 2 | | | | | | | | |
| | SV = 1 - [O81609_PEA] | | | | | | | | |
| B6CG41 | Non-specific lipid-transfer | 56.52 | 10 | 2 | 4 | 5 | 92 | 9.0 | 8.90 |
| | protein (Fragment) | | | | | | | | |
| | OS = Arachis hypogaea | | | | | | | | |
| | PE = 2 SV = 1 - | | | | | | | | |
| | [B6CG41_ARAHY] | | | | | | | | |
| B6CEX8 | Non-specific lipid-transfer | 38.79 | 10 | 2 | 4 | 5 | 116 | 11.6 | 9.14 |
| | protein OS = Arachis | | | | | | | | |
| | hypogaea PE = 2 SV = 1 - | | | | | | | | |
| | [B6CEX8_ARAHY] | | | | | | | | |
| A0A0B2PPT4 | Nuclease | 2.42 | 6 | 2 | 2 | 2 | 990 | 108.8 | 6.55 |
| | domain-containing protein | | | | | | | | |
| | 1 OS = Glycine soja | | | | | | | | |
| | GN = glysoja_011968 PE = 4 | | | | | | | | |
| | SV = 1 - | | | | | | | | |
| | [A0A0B2PPT4_GLYSO] | | | | | | | | |
| Q39839 | Nucleoside diphosphate | 16.11 | 4 | 1 | 2 | 3 | 149 | 16.4 | 6.32 |
| | kinase 1 OS = Glycine max | | | | | | | | |
| | PE = 2 SV = 1 - | | | | | | | | |
| | [NDK1_SOYBN] | | | | | | | | |
| Q45W80 | Nucleoside diphosphate | 49.66 | 28 | 5 | 6 | 11 | 149 | 16.4 | 6.80 |
| | kinase OS = Arachis | | | | | | | | |
| | hypogaea PE = 2 SV = 1 - | | | | | | | | |
| | [Q45W80_ARAHY] | | | | | | | | |
| Q45W87 | Oleosin 1 OS = Arachis | 24.82 | 1 | 1 | 3 | 6 | 137 | 14.3 | 10.08 |
| | hypogaea PE = 2 SV = 1 - | | | | | | | | |
| | [Q45W87_ARAHY] | | | | | | | | |
| Q647G5 | Oleosin 1 OS = Arachis | 34.32 | 3 | 5 | 5 | 22 | 169 | 17.7 | 9.58 |
| | hypogaea PE = 2 SV = 2 - | | | | | | | | |
| | [Q647G5_ARAHY] | | | | | | | | |
| Q45W86 | Oleosin 2 OS = Arachis | 24.82 | 1 | 1 | 3 | 6 | 137 | 14.3 | 9.99 |
| | hypogaea PE = 2 SV = 1 - | | | | | | | | |
| | [Q45W86_ARAHY] | | | | | | | | |
| Q647G3 | Oleosin OS = Arachis | 21.69 | 1 | 1 | 1 | 5 | 166 | 16.9 | 8.94 |
| | hypogaea PE = 2 SV = 1 - | | | | | | | | |
| | [Q647G3_ARAHY] | | | | | | | | |
| Q6J1J8 | Oleosin OS = Arachis | 25.00 | 2 | 1 | 4 | 9 | 176 | 18.4 | 9.54 |
| | hypogaea PE = 2 SV = 1 - | | | | | | | | |
| | [Q6J1J8_ARAHY] | | | | | | | | |
| A0A0B2Q9B8 | Oleosin OS = Glycine soja | 17.47 | 2 | 1 | 1 | 1 | 166 | 17.5 | 8.06 |
| | GN = glysoja_025116 PE = 3 | | | | | | | | |
| | SV = 1 - | | | | | | | | |
| | [A0A0B2Q9B8_GLYSO] | | | | | | | | |
| G7JS16 | Oleosin OS = Medicago | 5.96 | 4 | 1 | 1 | 1 | 151 | 16.4 | 9.11 |
| | truncatula | | | | | | | | |
| | GN = MTR_4g111770 | | | | | | | | |
| | PE = 4 SV = 1 - | | | | | | | | |
| | [G7JS16_MEDTR] | | | | | | | | |
| G7KPZ8 | Oleosin OS = Medicago | 9.33 | 1 | 1 | 1 | 1 | 193 | 20.7 | 8.65 |
| | truncatula | | | | | | | | |
| | GN = MTR_6g005040 | | | | | | | | |
| | PE = 4 SV = 1 - | | | | | | | | |
| | [G7KPZ8_MEDTR] | | | | | | | | |
| Q9AXI0 | Oleosin variant B | 27.84 | 1 | 1 | 4 | 11 | 176 | 18.4 | 9.54 |
| | OS = Arachis hypogaea | | | | | | | | |
| | PE = 2 SV = 1 - | | | | | | | | |
| | [Q9AXI0_ARAHY] | | | | | | | | |
| A9YWR9 | Oligopeptidase A | 2.86 | 4 | 1 | 1 | 1 | 700 | 79.1 | 5.49 |
| | OS = Medicago truncatula | | | | | | | | |
| | PE = 3 SV = 1 - | | | | | | | | |
| | [A9YWR9_MEDTR] | | | | | | | | |
| A0A0B2RFW5 | Omega-amidase NIT2 | 7.98 | 6 | 1 | 1 | 1 | 213 | 23.5 | 6.52 |
| | OS = Glycine soja | | | | | | | | |
| | GN = glysoja_035273 PE = 4 | | | | | | | | |
| | SV = 1 - | | | | | | | | |
| | [A0A0B2RFW5_GLYSO] | | | | | | | | |
| G7IUC7 | Outer membrane OMP85 | 1.34 | 5 | 1 | 1 | 1 | 672 | 73.0 | 8.56 |
| | family protein | | | | | | | | |
| | OS = Medicago truncatula | | | | | | | | |
| | GN = MTR_2g098110 | | | | | | | | |
| | PE = 4 SV = 1 - | | | | | | | | |
| | [G7IUC7_MEDTR] | | | | | | | | |
| A7LIS5 | Oxalate oxidase | 10.45 | 2 | 1 | 1 | 1 | 220 | 23.4 | 8.00 |
| | OS = Arachis hypogaea | | | | | | | | |
| | GN = OxOxs PE = 2 SV = 1 - | | | | | | | | |
| | [A7LIS5_ARAHY] | | | | | | | | |
| P29531 | P24 oleosin isoform B | 5.83 | 3 | 1 | 1 | 2 | 223 | 23.4 | 8.94 |
| | OS = Glycine max PE = 2 | | | | | | | | |
| | SV = 1 - [OLEO2_SOYBN] | | | | | | | | |
| A0A089ZXL7 | Peanut agglutinin variant | 35.53 | 1 | 4 | 7 | 47 | 273 | 29.4 | 6.05 |
| | OS = Arachis hypogaea | | | | | | | | |
| | PE = 2 SV = 1 - | | | | | | | | |
| | [A0A089ZXL7_ARAHY] | | | | | | | | |
| A0A072TSD1 | Pectinacetylesterase family | 3.05 | 3 | 1 | 1 | 1 | 393 | 43.2 | 8.47 |
| | protein OS = Medicago | | | | | | | | |
| | truncatula | | | | | | | | |
| | GN = MTR_8g072000 | | | | | | | | |
| | PE = 4 SV = 1 - | | | | | | | | |
| | [A0A072TSD1_MEDTR] | | | | | | | | |
| A0A0B2RGD4 | Peptidyl-prolyl cis-trans | 35.00 | 10 | 2 | 3 | 12 | 160 | 16.7 | 7.88 |
| | isomerase 1 OS = Glycine | | | | | | | | |
| | soja GN = glysoja_039168 | | | | | | | | |
| | PE = 3 SV = 1 - | | | | | | | | |
| | [A0A0B2RGD4_GLYSO] | | | | | | | | |
| S5WBX3 | Peptidyl-prolyl cis-trans | 29.07 | 11 | 2 | 3 | 3 | 172 | 18.4 | 7.83 |
| | isomerase | | | | | | | | |
| | OS = Aeschynomene filosa | | | | | | | | |
| | GN = CYP PE = 3 SV = 1 - | | | | | | | | |
| | [S5WBX3_9FABA] | | | | | | | | |
| A0A0A6ZDN9 | Peptidyl-prolyl cis-trans | 27.33 | 12 | 3 | 3 | 4 | 172 | 18.1 | 8.44 |
| | isomerase OS = Arachis | | | | | | | | |
| | hypogaea PE = 2 SV = 1 - | | | | | | | | |
| | [A0A0A6ZDN9_ARAHY] | | | | | | | | |
| K7KDU7 | Peptidyl-prolyl cis-trans | 12.75 | 12 | 1 | 1 | 1 | 102 | 11.5 | 8.47 |
| | isomerase OS = Glycine | | | | | | | | |
| | max PE = 4 SV = 1 - | | | | | | | | |
| | [K7KDU7_SOYBN] | | | | | | | | |
| A0A0B2RZG5 | Peptidyl-prolyl cis-trans | 6.00 | 2 | 1 | 1 | 1 | 250 | 26.7 | 8.16 |
| | isomerase OS = Glycine | | | | | | | | |
| | soja GN = glysoja_013467 | | | | | | | | |
| | PE = 3 SV = 1 - | | | | | | | | |
| | [A0A0B2RZG5_GLYSO] | | | | | | | | |
| A0A0B2QBL2 | Peptidyl-prolyl cis-trans | 7.14 | 7 | 1 | 1 | 1 | 112 | 12.0 | 6.01 |
| | isomerase OS = Glycine | | | | | | | | |
| | soja GN = glysoja_029863 | | | | | | | | |
| | PE = 4 SV = 1 - | | | | | | | | |
| | [A0A0B2QBL2_GLYSO] | | | | | | | | |
| A0A0B2RMD2 | Peptidyl-prolyl cis-trans | 3.92 | 1 | 1 | 1 | 1 | 204 | 21.9 | 8.85 |
| | isomerase OS = Glycine | | | | | | | | |
| | soja GN = glysoja_037234 | | | | | | | | |
| | PE = 3 SV = 1 - | | | | | | | | |
| | [A0A0B2RMD2_GLYSO] | | | | | | | | |
| I3SVV5 | Peptidyl-prolyl cis-trans | 15.52 | 1 | 1 | 1 | 1 | 174 | 18.5 | 8.43 |
| | isomerase OS = Lotus | | | | | | | | |
| | japonicus PE = 2 SV = 1 - | | | | | | | | |
| | [I3SVV5_LOTJA] | | | | | | | | |
| B4UWE2 | Perchloric acid soluble | 25.40 | 12 | 4 | 4 | 4 | 189 | 20.0 | 8.73 |
| | translation inhibitor | | | | | | | | |
| | protein OS = Arachis | | | | | | | | |
| | hypogaea PE = 2 SV = 1 - | | | | | | | | |
| | [B4UWE2_ARAHY] | | | | | | | | |
| H6VUU5 | Peroxiredoxin | 6.45 | 8 | 1 | 1 | 1 | 186 | 20.1 | 7.94 |
| | OS = Ammopiptanthus | | | | | | | | |
| | mongolicus PE = 2 SV = 1 - | | | | | | | | |
| | [H6VUU5_9FABA] | | | | | | | | |
| V5N806 | Peroxiredoxin-2E | 24.54 | 10 | 3 | 3 | 3 | 216 | 22.9 | 7.85 |
| | OS = Phaseolus vulgaris | | | | | | | | |
| | PE = 2 SV = 1 - | | | | | | | | |
| | [V5N806_PHAVU] | | | | | | | | |
| A0A072V9Z1 | Peroxisomal | 8.60 | 12 | 2 | 3 | 3 | 372 | 40.8 | 9.13 |
| | (S)-2-hydroxy-acid | | | | | | | | |
| | oxidase GLO1 | | | | | | | | |
| | OS = Medicago truncatula | | | | | | | | |
| | GN = MTR_2g070420 | | | | | | | | |
| | PE = 4 SV = 1 - | | | | | | | | |
| | [A0A072V9Z1_MEDTR] | | | | | | | | |
| B0M1B6 | Peroxisomal | 5.52 | 18 | 1 | 1 | 1 | 181 | 19.4 | 8.76 |
| | voltage-dependent | | | | | | | | |
| | anion-selective channel | | | | | | | | |
| | protein (Fragment) | | | | | | | | |
| | OS = Glycine max PE = 2 | | | | | | | | |
| | SV = 1 - | | | | | | | | |
| | [B0M1B6_SOYBN] | | | | | | | | |
| A0A0B2RDP7 | Peroxygenase 2 | 9.58 | 9 | 1 | 2 | 4 | 240 | 27.2 | 7.59 |
| | OS = Glycine soja | | | | | | | | |
| | GN = glysoja_035597 PE = 4 | | | | | | | | |
| | SV = 1 - | | | | | | | | |
| | [A0A0B2RDP7_GLYSO] | | | | | | | | |
| A0A072UGP9 | Phloem filament protein | 10.34 | 1 | 1 | 1 | 1 | 116 | 12.7 | 8.35 |
| | PP1 OS = Medicago | | | | | | | | |
| | truncatula | | | | | | | | |
| | GN = MTR_4g017620 | | | | | | | | |
| | PE = 4 SV = 1 - | | | | | | | | |
| | [A0A072UGP9_MEDTR] | | | | | | | | |
| A0A072UGX9 | Phosphatidylethanolamine- | 14.79 | 5 | 2 | 2 | 2 | 169 | 18.5 | 5.03 |
| | binding protein | | | | | | | | |
| | OS = Medicago truncatula | | | | | | | | |
| | GN = MTR_4g017650 | | | | | | | | |
| | PE = 4 SV = 1 - | | | | | | | | |
| | [A0A072UGX9_MEDTR] | | | | | | | | |
| A0A0B2RXX7 | Phosphoenolpyruvate | 8.70 | 21 | 3 | 7 | 7 | 966 | 110.6 | 6.42 |
| | carboxylase 2 OS = Glycine | | | | | | | | |
| | soja GN = glysoja_001428 | | | | | | | | |
| | PE = 3 SV = 1 - | | | | | | | | |
| | [A0A0B2RXX7_GLYSO] | | | | | | | | |
| C9W980 | Phosphoenolpyruvate | 12.80 | 18 | 5 | 9 | 9 | 969 | 110.8 | 6.47 |
| | carboxylase OS = Arachis | | | | | | | | |
| | hypogaea GN = PEPC4 | | | | | | | | |
| | PE = 2 SV = 1 - | | | | | | | | |
| | [C9W980_ARAHY] | | | | | | | | |
| Q9SM60 | Phosphoglucomutase, | 8.93 | 4 | 1 | 4 | 6 | 582 | 63.3 | 5.68 |
| | cytoplasmic OS = Pisum | | | | | | | | |
| | sativum GN = PGM1 PE = 2 | | | | | | | | |
| | SV = 1 - [PGMC_PEA] | | | | | | | | |
| B6RPS1 | Phosphogluconate | 19.08 | 47 | 1 | 2 | 2 | 131 | 14.9 | 9.52 |
| | dehydrogenase (Fragment) | | | | | | | | |
| | OS = Lotus purshianus | | | | | | | | |
| | GN = PGDH PE = 4 SV = 1 - | | | | | | | | |
| | [B6RPS1_LOTPU] | | | | | | | | |
| A0A0B2SQD7 | Phosphoglycerate kinase | 16.96 | 12 | 1 | 6 | 9 | 401 | 42.4 | 6.32 |
| | OS = Glycine soja | | | | | | | | |
| | GN = glysoja_019385 PE = 3 | | | | | | | | |
| | SV = 1 - | | | | | | | | |
| | [A0A0B2SQD7_GLYSO] | | | | | | | | |
| G7IT86 | Phosphoglycerate kinase | 14.46 | 13 | 1 | 6 | 7 | 401 | 42.4 | 6.01 |
| | OS = Medicago truncatula | | | | | | | | |
| | GN = MTR_2g066120 | | | | | | | | |
| | PE = 3 SV = 1 - | | | | | | | | |
| | [G7IT86_MEDTR] | | | | | | | | |
| V7BTD0 | Phosphoglycerate kinase | 15.71 | 10 | 1 | 6 | 7 | 401 | 42.2 | 6.61 |
| | OS = Phaseolus vulgaris | | | | | | | | |
| | GN = PHAVU_005G050800g | | | | | | | | |
| | PE = 3 SV = 1 - | | | | | | | | |
| | [V7BTDO_PHAVU] | | | | | | | | |
| Q2HWT8 | Phospholipase D | 5.95 | 7 | 3 | 3 | 3 | 807 | 91.8 | 5.86 |
| | OS = Arachis hypogaea | | | | | | | | |
| | GN = plda2 PE = 2 SV = 1 - | | | | | | | | |
| | [Q2HWT8_ARAHY] | | | | | | | | |
| P55195 | Phosphoribosylaminoimidazole | 3.77 | 8 | 1 | 1 | 1 | 557 | 60.9 | 6.42 |
| | carboxylase, | | | | | | | | |
| | chloroplastic (Fragment) | | | | | | | | |
| | OS = Vigna aconitifolia | | | | | | | | |
| | GN = PURKE PE = 2 SV = 1 - | | | | | | | | |
| | [PUR6_VIGAC] | | | | | | | | |
| A0A072V3H5 | Phytochrome-associated | 4.46 | 20 | 1 | 1 | 1 | 224 | 24.7 | 6.42 |
| | protein phosphatase type | | | | | | | | |
| | 2C OS = Medicago | | | | | | | | |
| | truncatula | | | | | | | | |
| | GN = MTR_3g105730 | | | | | | | | |
| | PE = 3 SV = 1 - | | | | | | | | |
| | [A0A072V3H5_MEDTR] | | | | | | | | |
| K7LQ70 | Poly [ADP-ribose] | 6.31 | 4 | 1 | 4 | 9 | 793 | 88.9 | 5.38 |
| | polymerase OS = Glycine | | | | | | | | |
| | max PE = 4 SV = 1 - | | | | | | | | |
| | [K7LQ70_SOYBN] | | | | | | | | |
| K7LTS6 | Poly [ADP-ribose] | 8.15 | 3 | 1 | 4 | 4 | 626 | 70.2 | 5.33 |
| | polymerase OS = Glycine | | | | | | | | |
| | max PE = 4 SV = 1 - | | | | | | | | |
| | [K7LTS6_SOYBN] | | | | | | | | |
| A0A072TXF8 | Polygalacturonase | 3.20 | 2 | 1 | 1 | 1 | 344 | 37.9 | 8.57 |
| | inhibitor OS = Medicago | | | | | | | | |
| | truncatula | | | | | | | | |
| | GN = MTR_7g033445 | | | | | | | | |
| | PE = 4 SV = 1 - | | | | | | | | |
| | [A0A072TXF8_MEDTR] | | | | | | | | |
| G7JF19 | PPR containing plant-like | 1.37 | 1 | 1 | 1 | 1 | 873 | 99.8 | 6.62 |
| | protein OS = Medicago | | | | | | | | |
| | truncatula | | | | | | | | |
| | GN = MTR_4g081220 | | | | | | | | |
| | PE = 4 SV = 2 - | | | | | | | | |
| | [G7JF19_MEDTR] | | | | | | | | |
| A0A0B2NT91 | Profilin OS = Glycine soja | 16.05 | 26 | 1 | 1 | 1 | 81 | 8.6 | 5.34 |
| | GN = glysoja_030764 PE = 3 | | | | | | | | |
| | SV = 1 - | | | | | | | | |
| | [A0A0B2NT91_GLYSO] | | | | | | | | |
| B7FJH0 | Prohibitin complex protein | 12.23 | 10 | 3 | 3 | 3 | 278 | 30.3 | 8.88 |
| | OS = Medicago truncatula | | | | | | | | |
| | GN = MTR_3g008250 | | | | | | | | |
| | PE = 2 SV = 1 - | | | | | | | | |
| | [B7FJH0_MEDTR] | | | | | | | | |
| B7FGZ3 | Proteasome subunit alpha | 12.87 | 19 | 2 | 2 | 2 | 171 | 18.7 | 4.83 |
| | type (Fragment) | | | | | | | | |
| | OS = Medicago truncatula | | | | | | | | |
| | PE = 2 SV = 1 - | | | | | | | | |
| | [B7FGZ3_MEDTR] | | | | | | | | |
| C6TG97 | Proteasome subunit alpha | 20.88 | 14 | 5 | 5 | 5 | 249 | 27.2 | 5.87 |
| | type OS = Glycine max | | | | | | | | |
| | PE = 2 SV = 1 - | | | | | | | | |
| | [C6TG97_SOYBN] | | | | | | | | |
| I1KTZ5 | Proteasome subunit alpha | 21.83 | 15 | 3 | 3 | 3 | 229 | 25.0 | 8.48 |
| | type OS = Glycine max | | | | | | | | |
| | PE = 3 SV = 1 - | | | | | | | | |
| | [I1KTZ5_SOYBN] | | | | | | | | |
| I3TA99 | Proteasome subunit alpha | 30.99 | 12 | 4 | 4 | 5 | 171 | 18.9 | 8.85 |
| | type OS = Lotus japonicus | | | | | | | | |
| | PE = 2 SV = 1 - | | | | | | | | |
| | [I3TA99_LOTJA] | | | | | | | | |
| Q1PCR5 | Proteasome subunit beta | 8.52 | 1 | 1 | 1 | 1 | 223 | 24.0 | 7.18 |
| | type (Fragment) | | | | | | | | |
| | OS = Arachis hypogaea | | | | | | | | |
| | PE = 2 SV = 1 - | | | | | | | | |
| | [Q1PCR5_ARAHY] | | | | | | | | |
| B7FGZ8 | Proteasome subunit beta | 15.52 | 10 | 3 | 3 | 3 | 232 | 24.9 | 5.55 |
| | type OS = Medicago | | | | | | | | |
| | truncatula | | | | | | | | |
| | GN = MTR_3g109210 | | | | | | | | |
| | PE = 2 SV = 1 - | | | | | | | | |
| | [B7FGZ8_MEDTR] | | | | | | | | |
| A0A0B2QS95 | Protein argonaute | 1.95 | 8 | 1 | 1 | 1 | 872 | 98.5 | 9.17 |
| | OS = Glycine soja | | | | | | | | |
| | GN = glysoja_014135 PE = 3 | | | | | | | | |
| | SV = 1 - | | | | | | | | |
| | [A0A0B2QS95_GLYSO] | | | | | | | | |
| A0A0B2S257 | Protein disulfide | 3.58 | 8 | 1 | 1 | 1 | 391 | 42.6 | 5.06 |
| | isomerase-like 2-3 | | | | | | | | |
| | OS = Glycine soja | | | | | | | | |
| | GN = glysoja_004169 PE = 3 | | | | | | | | |
| | SV = 1 - | | | | | | | | |
| | [A0A0B2S257_GLYSO] | | | | | | | | |
| G7KH66 | Protein disulfide | 2.76 | 1 | 1 | 1 | 1 | 435 | 47.4 | 5.57 |
| | isomerase-like protein | | | | | | | | |
| | OS = Medicago truncatula | | | | | | | | |
| | GN = MTR_5g084930 | | | | | | | | |
| | PE = 3 SV = 1 - | | | | | | | | |
| | [G7KH66_MEDTR] | | | | | | | | |
| B1Q2X5 | Protein | 2.00 | 3 | 1 | 1 | 1 | 551 | 61.6 | 4.84 |
| | disulfide-isomerase | | | | | | | | |
| | OS = Glycine max | | | | | | | | |
| | GN = PDIL-2 PE = 2 SV = 1 - | | | | | | | | |
| | [B1Q2X5_SOYBN] | | | | | | | | |
| A0A0B2SJW2 | Protein | 9.74 | 17 | 1 | 4 | 4 | 503 | 56.1 | 4.96 |
| | disulfide-isomerase | | | | | | | | |
| | OS = Glycine soja | | | | | | | | |
| | GN = glysoja_018373 PE = 3 | | | | | | | | |
| | SV = 1 - | | | | | | | | |
| | [A0A0B2SJW2_GLYSO] | | | | | | | | |
| A0A0B2RNG4 | Protein | 5.05 | 1 | 1 | 1 | 1 | 495 | 55.7 | 5.52 |
| | disulfide-isomerase | | | | | | | | |
| | OS = Glycine soja | | | | | | | | |
| | GN = glysoja_021715 PE = 3 | | | | | | | | |
| | SV = 1 - | | | | | | | | |
| | [A0A0B2RNG4_GLYSO] | | | | | | | | |
| A0A072V0J2 | Protein | 11.50 | 20 | 2 | 5 | 7 | 513 | 57.0 | 5.10 |
| | disulfide-isomerase | | | | | | | | |
| | OS = Medicago truncatula | | | | | | | | |
| | GN = MTR_3g088220 | | | | | | | | |
| | PE = 3 SV = 1 - | | | | | | | | |
| | [A0A072V0J2_MEDTR] | | | | | | | | |
| A0A0B2PFH8 | Protein LHY OS = Glycine | 1.51 | 1 | 1 | 1 | 1 | 661 | 72.2 | 6.00 |
| | soja GN = glysoja_021216 | | | | | | | | |
| | PE = 4 SV = 1 - | | | | | | | | |
| | [A0A0B2PFH8_GLYSO] | | | | | | | | |
| A0A0B2R2K5 | Protein transport protein | 14.29 | 13 | 1 | 1 | 1 | 70 | 7.6 | 10.96 |
| | Sec61 subunit beta | | | | | | | | |
| | OS = Glycine soja | | | | | | | | |
| | GN = glysoja_043651 PE = 4 | | | | | | | | |
| | SV = 1 - | | | | | | | | |
| | [A0A0B2R2K5_GLYSO] | | | | | | | | |
| G7J8C0 | Puromycin-sensitive | 4.68 | 8 | 2 | 4 | 6 | 876 | 98.9 | 5.69 |
| | aminopeptidase-like | | | | | | | | |
| | protein OS = Medicago | | | | | | | | |
| | truncatula | | | | | | | | |
| | GN = MTR_3g104720 | | | | | | | | |
| | PE = 4 SV = 1 - | | | | | | | | |
| | [G7J8C0_MEDTR] | | | | | | | | |
| A0A0B2PIU9 | Putative 26S proteasome | 3.88 | 9 | 1 | 1 | 1 | 309 | 34.7 | 6.28 |
| | non-ATPase regulatory | | | | | | | | |
| | subunit 7 OS = Glycine soja | | | | | | | | |
| | GN = glysoja_019751 PE = 4 | | | | | | | | |
| | SV = 1 - | | | | | | | | |
| | [A0A0B2PIU9_GLYSO] | | | | | | | | |
| E3P863 | Putative | 7.73 | 1 | 1 | 1 | 1 | 181 | 20.6 | 11.46 |
| | arginine/serine-rich | | | | | | | | |
| | splicing factor | | | | | | | | |
| | OS = Arachis diogoi | | | | | | | | |
| | GN = RSZ PE = 2 SV = 2 - | | | | | | | | |
| | [E3P863_9FABA] | | | | | | | | |
| Q39852 | Putative ATP synthase | 6.15 | 13 | 1 | 1 | 1 | 179 | 20.2 | 8.76 |
| | subunit OS = Glycine max | | | | | | | | |
| | PE = 4 SV = 1 - | | | | | | | | |
| | [Q39852_SOYBN] | | | | | | | | |
| A0A0B2S7M5 | Putative calcium-binding | 10.48 | 10 | 1 | 1 | 1 | 105 | 11.9 | 7.18 |
| | protein CML7 | | | | | | | | |
| | OS = Glycine soja | | | | | | | | |
| | GN = glysoja_018959 PE = 4 | | | | | | | | |
| | SV = 1 - | | | | | | | | |
| | [A0A0B2S7M5_GLYSO] | | | | | | | | |
| A0A089GJT0 | Putative glutathione | 3.65 | 2 | 1 | 1 | 1 | 219 | 25.6 | 6.25 |
| | S-transferase | | | | | | | | |
| | OS = Medicago sativa PE = 2 | | | | | | | | |
| | SV = 1 - | | | | | | | | |
| | [A0A089GJT0_MEDSA] | | | | | | | | |
| Q2PEX3 | Putative HEAT SHOCK | 18.20 | 9 | 0 | 7 | 8 | 478 | 55.4 | 5.24 |
| | PROTEIN 81-2 | | | | | | | | |
| | (Fragment) OS = Trifolium | | | | | | | | |
| | pratense PE = 2 SV = 1 - | | | | | | | | |
| | [Q2PEX3_TRIPR] | | | | | | | | |
| Q1PCR4 | Putative IN2-1 protein | 10.79 | 1 | 1 | 1 | 1 | 139 | 16.1 | 5.36 |
| | (Fragment) OS = Arachis | | | | | | | | |
| | hypogaea PE = 2 SV = 1 - | | | | | | | | |
| | [Q1PCR4_ARAHY] | | | | | | | | |
| B4UWB3 | Putative L24 ribosomal | 28.77 | 13 | 4 | 4 | 6 | 146 | 16.8 | 10.90 |
| | protein OS = Arachis | | | | | | | | |
| | hypogaea PE = 2 SV = 1 - | | | | | | | | |
| | [B4UWB3_ARAHY] | | | | | | | | |
| Q850G6 | Putative lea 4 (Fragment) | 22.47 | 35 | 2 | 2 | 2 | 89 | 9.7 | 8.94 |
| | OS = Arachis hypogaea | | | | | | | | |
| | GN = Gsi-22 PE = 2 SV = 1 - | | | | | | | | |
| | [Q850G6_ARAHY] | | | | | | | | |
| B4UWD3 | Putative mitochondrial | 29.69 | 1 | 2 | 2 | 2 | 64 | 7.4 | 5.40 |
| | ATP synthase (Fragment) | | | | | | | | |
| | OS = Arachis hypogaea | | | | | | | | |
| | PE = 2 SV = 1 - | | | | | | | | |
| | [B4UWD3_ARAHY] | | | | | | | | |
| C6SW87 | Putative uncharacterized | 11.58 | 7 | 1 | 1 | 1 | 190 | 22.0 | 9.77 |
| | protein (Fragment) | | | | | | | | |
| | OS = Glycine max PE = 2 | | | | | | | | |
| | SV = 1 - | | | | | | | | |
| | [C6SW87_SOYBN] | | | | | | | | |
| C6SZN9 | Putative uncharacterized | 4.74 | 7 | 1 | 1 | 1 | 211 | 22.8 | 9.07 |
| | protein (Fragment) | | | | | | | | |
| | OS = Glycine max PE = 2 | | | | | | | | |
| | SV = 1 - | | | | | | | | |
| | [C6SZN9_SOYBN] | | | | | | | | |
| C6T7C4 | Putative uncharacterized | 4.08 | 8 | 1 | 1 | 1 | 245 | 26.0 | 5.83 |
| | protein (Fragment) | | | | | | | | |
| | OS = Glycine max PE = 2 | | | | | | | | |
| | SV = 1 - | | | | | | | | |
| | [C6T7C4_SOYBN] | | | | | | | | |
| C6T9I0 | Putative uncharacterized | 3.57 | 6 | 1 | 1 | 1 | 280 | 30.9 | 4.84 |
| | protein (Fragment) | | | | | | | | |
| | OS = Glycine max PE = 2 | | | | | | | | |
| | SV = 1 - [C6T9I0_SOYBN] | | | | | | | | |
| B7FI35 | Putative uncharacterized | 4.40 | 2 | 1 | 1 | 1 | 318 | 36.6 | 5.90 |
| | protein (Fragment) | | | | | | | | |
| | OS = Medicago truncatula | | | | | | | | |
| | PE = 2 SV = 1 - | | | | | | | | |
| | [B7FI35_MEDTR] | | | | | | | | |
| B7FII6 | Putative uncharacterized | 4.88 | 3 | 1 | 1 | 1 | 328 | 35.5 | 4.98 |
| | protein (Fragment) | | | | | | | | |
| | OS = Medicago truncatula | | | | | | | | |
| | PE = 2 SV = 1 - | | | | | | | | |
| | [B7FII6_MEDTR] | | | | | | | | |
| B7FLB3 | Putative uncharacterized | 5.73 | 11 | 1 | 1 | 1 | 227 | 26.0 | 8.56 |
| | protein (Fragment) | | | | | | | | |
| | OS = Medicago truncatula | | | | | | | | |
| | PE = 2 SV = 1 - | | | | | | | | |
| | [B7FLB3_MEDTR] | | | | | | | | |
| B7FLE3 | Putative uncharacterized | 4.56 | 20 | 1 | 1 | 1 | 263 | 28.6 | 8.65 |
| | protein (Fragment) | | | | | | | | |
| | OS = Medicago truncatula | | | | | | | | |
| | PE = 2 SV = 1 - | | | | | | | | |
| | [B7FLE3_MEDTR] | | | | | | | | |
| B4UWA3 | Putative uncharacterized | 4.97 | 1 | 1 | 1 | 1 | 181 | 20.2 | 5.00 |
| | protein OS = Arachis | | | | | | | | |
| | hypogaea PE = 2 SV = 1 - | | | | | | | | |
| | [B4UWA3_ARAHY] | | | | | | | | |
| C6SVE3 | Putative uncharacterized | 51.65 | 22 | 3 | 3 | 4 | 91 | 10.0 | 9.11 |
| | protein OS = Glycine max | | | | | | | | |
| | PE = 2 SV = 1 - | | | | | | | | |
| | [C6SVE3_SOYBN] | | | | | | | | |
| C6SW37 | Putative uncharacterized | 12.50 | 7 | 1 | 1 | 1 | 64 | 7.1 | 9.54 |
| | protein OS = Glycine max | | | | | | | | |
| | PE = 2 SV = 1 - | | | | | | | | |
| | [C6SW37_SOYBN] | | | | | | | | |
| C6SW70 | Putative uncharacterized | 12.86 | 13 | 1 | 3 | 3 | 241 | 28.2 | 9.88 |
| | protein OS = Glycine max | | | | | | | | |
| | PE = 2 SV = 1 - | | | | | | | | |
| | [C6SW70_SOYBN] | | | | | | | | |
| C6SY23 | Putative uncharacterized | 6.11 | 10 | 1 | 1 | 1 | 180 | 19.8 | 8.43 |
| | protein OS = Glycine max | | | | | | | | |
| | PE = 2 SV = 1 - | | | | | | | | |
| | [C6SY23_SOYBN] | | | | | | | | |
| C6SYA7 | Putative uncharacterized | 7.59 | 3 | 1 | 1 | 1 | 158 | 18.5 | 5.24 |
| | protein OS = Glycine max | | | | | | | | |
| | PE = 2 SV = 1 - | | | | | | | | |
| | [C6SYA7_SOYBN] | | | | | | | | |
| C6SZF9 | Putative uncharacterized | 13.85 | 33 | 2 | 3 | 3 | 231 | 25.7 | 10.20 |
| | protein OS = Glycine max | | | | | | | | |
| | PE = 2 SV = 1 - | | | | | | | | |
| | [C6SZF9_SOYBN] | | | | | | | | |
| C6T5L3 | Putative uncharacterized | 18.80 | 16 | 2 | 2 | 2 | 117 | 13.3 | 9.94 |
| | protein OS = Glycine max | | | | | | | | |
| | PE = 2 SV = 1 - | | | | | | | | |
| | [C6T5L3_SOYBN] | | | | | | | | |
| C6T7G4 | Putative uncharacterized | 6.33 | 42 | 1 | 2 | 2 | 395 | 44.1 | 7.65 |
| | protein OS = Glycine max | | | | | | | | |
| | PE = 2 SV = 1 - | | | | | | | | |
| | [C6T7G4_SOYBN] | | | | | | | | |
| C6T7U2 | Putative uncharacterized | 31.49 | 3 | 1 | 11 | 21 | 470 | 51.5 | 5.58 |
| | protein OS = Glycine max | | | | | | | | |
| | PE = 2 SV = 1 - | | | | | | | | |
| | [C6T7U2_SOYBN] | | | | | | | | |
| C6T8Y3 | Putative uncharacterized | 6.08 | 21 | 1 | 1 | 1 | 181 | 19.8 | 6.77 |
| | protein OS = Glycine max | | | | | | | | |
| | PE = 2 SV = 1 - | | | | | | | | |
| | [C6T8Y3_SOYBN] | | | | | | | | |
| C6T8Y9 | Putative uncharacterized | 8.18 | 19 | 1 | 1 | 1 | 159 | 17.1 | 7.81 |
| | protein OS = Glycine max | | | | | | | | |
| | PE = 2 SV = 1 - | | | | | | | | |
| | [C6T8Y9_SOYBN] | | | | | | | | |
| C6TCG3 | Putative uncharacterized | 4.89 | 15 | 1 | 1 | 1 | 184 | 20.3 | 5.47 |
| | protein OS = Glycine max | | | | | | | | |
| | PE = 2 SV = 1 - | | | | | | | | |
| | [C6TCG3_SOYBN] | | | | | | | | |
| C6TDC8 | Putative uncharacterized | 5.36 | 8 | 1 | 1 | 1 | 224 | 24.4 | 6.33 |
| | protein OS = Glycine max | | | | | | | | |
| | PE = 2 SV = 1 - | | | | | | | | |
| | [C6TDC8_SOYBN] | | | | | | | | |
| C6TDF5 | Putative uncharacterized | 17.32 | 23 | 2 | 5 | 5 | 381 | 41.9 | 7.36 |
| | protein OS = Glycine max | | | | | | | | |
| | PE = 2 SV = 1 - | | | | | | | | |
| | [C6TDF5_SOYBN] | | | | | | | | |
| C6TI42 | Putative uncharacterized | 16.36 | 1 | 1 | 1 | 1 | 55 | 6.3 | 4.61 |
| | protein OS = Glycine max | | | | | | | | |
| | PE = 2 SV = 1 - | | | | | | | | |
| | [C6TI42_SOYBN] | | | | | | | | |
| C6TID1 | Putative uncharacterized | 3.49 | 7 | 1 | 1 | 1 | 315 | 35.1 | 4.89 |
| | protein OS = Glycine max | | | | | | | | |
| | PE = 2 SV = 1 - | | | | | | | | |
| | [C6TID1_SOYBN] | | | | | | | | |
| C6TIK9 | Putative uncharacterized | 10.00 | 27 | 1 | 3 | 3 | 280 | 30.6 | 7.93 |
| | protein OS = Glycine max | | | | | | | | |
| | PE = 2 SV = 1 - | | | | | | | | |
| | [C6TIK9_SOYBN] | | | | | | | | |
| C6TIR2 | Putative uncharacterized | 35.57 | 30 | 5 | 5 | 5 | 149 | 16.9 | 4.27 |
| | protein OS = Glycine max | | | | | | | | |
| | PE = 2 SV = 1 - | | | | | | | | |
| | [C6TIR2_SOYBN] | | | | | | | | |
| B7FHC4 | Putative uncharacterized | 12.70 | 17 | 1 | 3 | 3 | 244 | 28.4 | 9.89 |
| | protein OS = Medicago | | | | | | | | |
| | truncatula PE = 2 SV = 1 - | | | | | | | | |
| | [B7FHC4_MEDTR] | | | | | | | | |
| B7FK48 | Putative uncharacterized | 4.35 | 3 | 1 | 1 | 1 | 276 | 29.2 | 9.23 |
| | protein OS = Medicago | | | | | | | | |
| | truncatula PE = 2 SV = 1 - | | | | | | | | |
| | [B7FK48_MEDTR] | | | | | | | | |
| B7FKP5 | Putative uncharacterized | 6.45 | 6 | 1 | 1 | 1 | 186 | 20.1 | 9.57 |
| | protein OS = Medicago | | | | | | | | |
| | truncatula PE = 2 SV = 1 - | | | | | | | | |
| | [B7FKP5_MEDTR] | | | | | | | | |
| B7FKR5 | Putative uncharacterized | 21.75 | 16 | 6 | 6 | 6 | 285 | 31.5 | 6.93 |
| | protein OS = Medicago | | | | | | | | |
| | truncatula PE = 2 SV = 1 - | | | | | | | | |
| | [B7FKR5_MEDTR] | | | | | | | | |
| B7FLH4 | Putative uncharacterized | 2.62 | 8 | 1 | 1 | 1 | 381 | 41.8 | 5.86 |
| | protein OS = Medicago | | | | | | | | |
| | truncatula PE = 2 SV = 1 - | | | | | | | | |
| | [B7FLH4_MEDTR] | | | | | | | | |
| B7FLR8 | Putative uncharacterized | 1.91 | 8 | 1 | 1 | 1 | 577 | 65.0 | 8.57 |
| | protein OS = Medicago | | | | | | | | |
| | truncatula PE = 2 SV = 1 - | | | | | | | | |
| | [B7FLR8_MEDTR] | | | | | | | | |
| B7FLW3 | Putative uncharacterized | 4.42 | 26 | 1 | 1 | 1 | 362 | 40.4 | 7.47 |
| | protein OS = Medicago | | | | | | | | |
| | truncatula PE = 2 SV = 1 - | | | | | | | | |
| | [B7FLW3_MEDTR] | | | | | | | | |
| B7FME1 | Putative uncharacterized | 9.09 | 3 | 1 | 1 | 1 | 143 | 16.2 | 8.97 |
| | protein OS = Medicago | | | | | | | | |
| | truncatula PE = 2 SV = 1 - | | | | | | | | |
| | [B7FME1_MEDTR] | | | | | | | | |
| A0A0B2Q4Q7 | Pyrophosphate-energized | 7.38 | 31 | 4 | 4 | 4 | 732 | 76.6 | 5.33 |
| | vacuolar membrane proton | | | | | | | | |
| | pump OS = Glycine soja | | | | | | | | |
| | GN = glysoja_0 11651 PE = 3 | | | | | | | | |
| | SV = 1 - | | | | | | | | |
| | [A0A0B2Q4Q7_GLYSO] | | | | | | | | |
| A0A0B2R6L6 | Pyrophosphate-fructose | 5.80 | 10 | 1 | 1 | 1 | 224 | 24.2 | 6.79 |
| | 6-phosphate | | | | | | | | |
| | 1-phosphotransferase | | | | | | | | |
| | subunit beta OS = Glycine | | | | | | | | |
| | soja GN = glysoja_036010 | | | | | | | | |
| | PE = 4 SV = 1 - | | | | | | | | |
| | [A0A0B2R6L6_GLYSO] | | | | | | | | |
| G7IA37 | Pyrophosphate-fructose-6- | 2.35 | 1 | 1 | 1 | 1 | 597 | 65.3 | 6.67 |
| | phosphate | | | | | | | | |
| | 1-phosphotransferase | | | | | | | | |
| | OS = Medicago truncatula | | | | | | | | |
| | GN = MTR_1g035230 | | | | | | | | |
| | PE = 3 SV = 2 - | | | | | | | | |
| | [G7IA37_MEDTR] | | | | | | | | |
| P51851 | Pyruvate decarboxylase 2 | 8.40 | 11 | 3 | 3 | 3 | 405 | 44.1 | 6.68 |
| | (Fragment) OS = Pisum | | | | | | | | |
| | sativum GN = PDC2 PE = 2 | | | | | | | | |
| | SV = 1 - [PDC2_PEA] | | | | | | | | |
| A0A0B2S404 | Pyruvate dehydrogenase | 7.50 | 10 | 2 | 2 | 2 | 360 | 38.7 | 5.96 |
| | E1 component subunit | | | | | | | | |
| | beta, mitochondrial | | | | | | | | |
| | OS = Glycine soja | | | | | | | | |
| | GN = glysoja_009377 PE = 4 | | | | | | | | |
| | SV = 1 - | | | | | | | | |
| | [A0A0B2S404_GLYSO] | | | | | | | | |
| I1NAH7 | Pyruvate kinase | 13.47 | 30 | 4 | 5 | 5 | 527 | 57.5 | 7.50 |
| | OS = Glycine max PE = 3 | | | | | | | | |
| | SV = 1 - | | | | | | | | |
| | [I1NAH7_SOYBN] | | | | | | | | |
| A0A0B2P7Y1 | Ran-binding protein 1 like | 16.91 | 9 | 2 | 2 | 2 | 136 | 15.1 | 4.83 |
| | b OS = Glycine soja | | | | | | | | |
| | GN = glysoja_046443 PE = 4 | | | | | | | | |
| | SV = 1 - | | | | | | | | |
| | [A0A0B2P7Y1_GLYSO] | | | | | | | | |
| A0A0B2R712 | Remorin OS = Glycine soja | 4.92 | 3 | 1 | 1 | 1 | 183 | 20.2 | 8.31 |
| | GN = glysoja_005099 PE = 4 | | | | | | | | |
| | SV = 1 - | | | | | | | | |
| | [A0A0B2R712_GLYSO] | | | | | | | | |
| A0A0B2QNR7 | Reticulon-like protein | 3.35 | 14 | 1 | 1 | 1 | 239 | 27.1 | 6.27 |
| | OS = Glycine soja | | | | | | | | |
| | GN = glysoja_027683 PE = 4 | | | | | | | | |
| | SV = 1 - | | | | | | | | |
| | [A0A0B2QNR7_GLYSO] | | | | | | | | |
| A0A072UVR7 | Retinoid-inducible serine | 2.84 | 1 | 1 | 1 | 1 | 458 | 51.1 | 7.75 |
| | carboxypeptidase | | | | | | | | |
| | OS = Medicago truncatula | | | | | | | | |
| | GN = MTR_4g056520 | | | | | | | | |
| | PE = 4 SV = 1 - | | | | | | | | |
| | [A0A072UVR7_MEDTR] | | | | | | | | |
| G7L1U4 | Ribose-5-phosphate | 5.00 | 5 | 1 | 1 | 1 | 280 | 29.9 | 5.59 |
| | isomerase A | | | | | | | | |
| | OS = Medicago truncatula | | | | | | | | |
| | GN = MTR_7g114900 | | | | | | | | |
| | PE = 3 SV = 1 - | | | | | | | | |
| | [G7L1U4_MEDTR] | | | | | | | | |
| A0A0A6ZDR7 | Ribosomal protein L15 | 19.61 | 21 | 4 | 4 | 5 | 204 | 24.2 | 11.44 |
| | OS = Arachis hypogaea | | | | | | | | |
| | PE = 2 SV = 1 - | | | | | | | | |
| | [A0A0A6ZDR7_ARAHY] | | | | | | | | |
| A0A0B2R3J3 | Ribosomal protein L19 | 20.10 | 40 | 4 | 4 | 4 | 204 | 23.7 | 11.21 |
| | OS = Glycine soja | | | | | | | | |
| | GN = glysoja_038842 PE = 3 | | | | | | | | |
| | SV = 1 - | | | | | | | | |
| | [A0A0B2R3J3_GLYSO] | | | | | | | | |
| C6SVL1 | Ribosomal protein | 17.13 | 28 | 3 | 3 | 4 | 216 | 24.6 | 9.82 |
| | OS = Glycine max PE = 2 | | | | | | | | |
| | SV = 1 - | | | | | | | | |
| | [C6SVL1_SOYBN] | | | | | | | | |
| X2D3D5 | Ribosomal protein S18 | 27.55 | 7 | 1 | 2 | 2 | 98 | 11.2 | 9.51 |
| | (Fragment) OS = Trifolium | | | | | | | | |
| | repens GN = rpS18 PE = 2 | | | | | | | | |
| | SV = 1 - [X2D3D5_TRIRP] | | | | | | | | |
| O20356 | Ribulose 1,5-bisphosphate | 17.42 | 1643 | 1 | 7 | 13 | 465 | 51.5 | 6.64 |
| | carboxylase-oxygenase | | | | | | | | |
| | large subunit (Fragment) | | | | | | | | |
| | OS = Arachis hypogaea | | | | | | | | |
| | GN = rbcL PE = 3 SV = 1 - | | | | | | | | |
| | [O20356_ARAHY] | | | | | | | | |
| M1QD53 | Ribulose bisphosphate | 18.00 | 762 | 1 | 3 | 5 | 200 | 22.5 | 5.69 |
| | carboxylase large chain | | | | | | | | |
| | (Fragment) OS = Erythrina | | | | | | | | |
| | subumbrans GN = rbcL | | | | | | | | |
| | PE = 3 SV = 1 - | | | | | | | | |
| | [M1QD53_9FABA] | | | | | | | | |
| L7R5S0 | Ribulose bisphosphate | 17.28 | 27 | 1 | 5 | 9 | 301 | 33.3 | 6.99 |
| | carboxylase large chain | | | | | | | | |
| | (Fragment) OS = Medicago | | | | | | | | |
| | polymorpha GN = rbcL | | | | | | | | |
| | PE = 3 SV = 1 - | | | | | | | | |
| | [L7R5S0_9FABA] | | | | | | | | |
| I3S2B8 | Ribulose bisphosphate | 9.25 | 2 | 2 | 2 | 2 | 173 | 19.7 | 8.73 |
| | carboxylase small chain | | | | | | | | |
| | OS = Lotus japonicus PE = 2 | | | | | | | | |
| | SV = 1 - [I3S2B8_LOTJA] | | | | | | | | |
| G7JVD3 | Ribulose bisphosphate | 10.40 | 1 | 2 | 2 | 5 | 173 | 19.7 | 8.91 |
| | carboxylase small chain | | | | | | | | |
| | OS = Medicago truncatula | | | | | | | | |
| | GN = MTR_4g127870 | | | | | | | | |
| | PE = 2 SV = 1 - | | | | | | | | |
| | [G7JVD3_MEDTR] | | | | | | | | |
| G9C066 | Ripening-related protein | 12.16 | 28 | 1 | 1 | 1 | 74 | 8.6 | 7.64 |
| | (Fragment) OS = Oxytropis | | | | | | | | |
| | maydelliana PE = 4 SV = 1 - | | | | | | | | |
| | [G9C066_9FABA] | | | | | | | | |
| A0A0B2QVA3 | RuBisCO large | 13.92 | 9 | 1 | 6 | 6 | 546 | 58.1 | 5.24 |
| | subunit-binding protein | | | | | | | | |
| | subunit beta, chloroplastic | | | | | | | | |
| | OS = Glycine soja | | | | | | | | |
| | GN = glysoja_049054 PE = 3 | | | | | | | | |
| | SV = 1 - | | | | | | | | |
| | [A0A0B2QVA3_GLYSO] | | | | | | | | |
| P08927 | RuBisCO large | 12.94 | 3 | 1 | 6 | 6 | 595 | 62.9 | 6.04 |
| | subunit-binding protein | | | | | | | | |
| | subunit beta, chloroplastic | | | | | | | | |
| | OS = Pisum sativum PE = 1 | | | | | | | | |
| | SV = 2 - [RUBB_PEA] | | | | | | | | |
| C6K8D1 | Seed biotinylated protein | 6.69 | 6 | 4 | 4 | 5 | 643 | 67.9 | 6.58 |
| | 68 kDa isoform | | | | | | | | |
| | OS = Glycine max PE = 2 | | | | | | | | |
| | SV = 1 - | | | | | | | | |
| | [C6K8D1_SOYBN] | | | | | | | | |
| N1NKG9 | Seed maturation protein | 67.58 | 2 | 8 | 8 | 17 | 256 | 26.0 | 5.30 |
| | OS = Arachis hypogaea | | | | | | | | |
| | GN = ARAX_AHF417E07- | | | | | | | | |
| | 003 PE = 4 SV = 1 - | | | | | | | | |
| | [N1NKG9_ARAHY] | | | | | | | | |
| Q9SEK9 | Seed maturation protein | 6.32 | 3 | 1 | 1 | 1 | 253 | 25.7 | 5.10 |
| | PM25 OS = Glycine max | | | | | | | | |
| | GN = PM25 PE = 2 SV = 1 - | | | | | | | | |
| | [Q9SEK9_SOYBN] | | | | | | | | |
| Q9XES7 | Seed maturation protein | 2.65 | 7 | 1 | 1 | 1 | 339 | 38.0 | 9.38 |
| | PM27 OS = Glycine max | | | | | | | | |
| | GN = PM27 PE = 2 SV = 1 - | | | | | | | | |
| | [Q9XES7_SOYBN] | | | | | | | | |
| Q9XET1 | Seed maturation protein | 12.42 | 1 | 1 | 1 | 3 | 153 | 17.7 | 6.55 |
| | PM31 OS = Glycine max | | | | | | | | |
| | GN = PM31 PE = 2 SV = 1 - | | | | | | | | |
| | [Q9XET1_SOYBN] | | | | | | | | |
| A0A0B2P0S6 | Seed maturation protein | 5.85 | 6 | 1 | 1 | 1 | 188 | 21.5 | 5.66 |
| | PM36 OS = Glycine soja | | | | | | | | |
| | GN = glysoja_009766 PE = 4 | | | | | | | | |
| | SV = 1 - | | | | | | | | |
| | [A0A0B2P0S6_GLYSO] | | | | | | | | |
| A0A072U9M9 | Seed maturation protein | 0.99 | 1 | 1 | 1 | 1 | 1109 | 119.4 | 6.37 |
| | PM39, putative | | | | | | | | |
| | OS = Medicago truncatula | | | | | | | | |
| | GN = MTR_6g045273 | | | | | | | | |
| | PE = 4 SV = 1 - | | | | | | | | |
| | [A0A072U9M9_MEDTR] | | | | | | | | |
| Q93YH7 | Selenium binding protein | 2.30 | 3 | 1 | 1 | 1 | 478 | 53.0 | 6.02 |
| | (Fragment) OS = Glycine | | | | | | | | |
| | max GN = sbp PE = 2 SV = 1 - | | | | | | | | |
| | [Q93YH7_SOYBN] | | | | | | | | |
| A0A0A6ZDR9 | Serine | 27.60 | 22 | 10 | 10 | 13 | 471 | 52.0 | 7.28 |
| | hydroxymethyltransferase | | | | | | | | |
| | OS = Arachis hypogaea | | | | | | | | |
| | PE = 2 SV = 1 - | | | | | | | | |
| | [A0A0A6ZDR9_ARAHY] | | | | | | | | |
| Q2VMU0 | Serine protease inhibitor | 25.23 | 1 | 1 | 1 | 2 | 107 | 11.5 | 5.38 |
| | OS = Arachis hypogaea | | | | | | | | |
| | PE = 2 SV = 1 - | | | | | | | | |
| | [Q2VMU0_ARAHY] | | | | | | | | |
| A0A0B2SG37 | Signal peptidase complex | 10.96 | 5 | 1 | 1 | 1 | 146 | 16.8 | 9.00 |
| | subunit 3B OS = Glycine | | | | | | | | |
| | soja GN = glysoja_001855 | | | | | | | | |
| | PE = 4 SV = 1 - | | | | | | | | |
| | [A0A0B2SG37_GLYSO] | | | | | | | | |
| Q9XF99 | Skp1 (Fragment) | 9.80 | 13 | 1 | 1 | 1 | 153 | 17.3 | 4.65 |
| | OS = Medicago sativa | | | | | | | | |
| | GN = SKP1 PE = 2 SV = 1 - | | | | | | | | |
| | [Q9XF99_MEDSA] | | | | | | | | |
| A0A0B2QRE4 | SNF1-related protein | 7.53 | 5 | 2 | 2 | 2 | 385 | 42.0 | 5.96 |
| | kinase regulatory subunit | | | | | | | | |
| | gamma-like PV42a | | | | | | | | |
| | OS = Glycine soja | | | | | | | | |
| | GN = glysoja_024947 PE = 4 | | | | | | | | |
| | SV = 1 - | | | | | | | | |
| | [A0A0B2QRE4_GLYSO] | | | | | | | | |
| G7JHI5 | SPFH domain/band 7 | 2.49 | 1 | 1 | 1 | 1 | 361 | 40.9 | 6.05 |
| | family protein | | | | | | | | |
| | OS = Medicago truncatula | | | | | | | | |
| | GN = MTR_4g083100 | | | | | | | | |
| | PE = 4 SV = 1 - | | | | | | | | |
| | [G7JHI5_MEDTR] | | | | | | | | |
| A7LB60 | Steroleosin A OS = Arachis | 43.55 | 2 | 13 | 13 | 22 | 349 | 38.7 | 6.93 |
| | hypogaea GN = STO-A | | | | | | | | |
| | PE = 2 SV = 1 - | | | | | | | | |
| | [A7LB60_ARAHY] | | | | | | | | |
| A7LB59 | Steroleosin-B OS = Arachis | 24.08 | 6 | 8 | 8 | 18 | 353 | 39.6 | 6.64 |
| | hypogaea GN = STO-B | | | | | | | | |
| | PE = 2 SV = 1 - | | | | | | | | |
| | [A7LB59_ARAHY] | | | | | | | | |
| G7KG90 | Stress-inducible protein, | 3.96 | 4 | 1 | 2 | 2 | 581 | 65.3 | 5.94 |
| | putative OS = Medicago | | | | | | | | |
| | truncatula | | | | | | | | |
| | GN = MTR_5g012030 | | | | | | | | |
| | PE = 4 SV = 1 - | | | | | | | | |
| | [G7KG90_MEDTR] | | | | | | | | |
| A0A0B2S350 | Stromal 70 kDa heat | 11.46 | 10 | 7 | 7 | 8 | 611 | 65.4 | 4.98 |
| | shock-related protein, | | | | | | | | |
| | chloroplastic OS = Glycine | | | | | | | | |
| | soja GN = glysoja_020753 | | | | | | | | |
| | PE = 3 SV = 1 - | | | | | | | | |
| | [A0A0B2S350_GLYSO] | | | | | | | | |
| I1JCQ7 | Succinate dehydrogenase | 8.36 | 15 | 4 | 4 | 5 | 634 | 69.7 | 6.76 |
| | [ubiquinone] flavoprotein | | | | | | | | |
| | subunit, mitochondrial | | | | | | | | |
| | OS = Glycine max PE = 3 | | | | | | | | |
| | SV = 1 - [I1JCQ7_SOYBN] | | | | | | | | |
| I1MLQ8 | Succinate dehydrogenase | 8.60 | 6 | 2 | 2 | 2 | 279 | 31.5 | 8.70 |
| | [ubiquinone] iron-sulfur | | | | | | | | |
| | subunit, mitochondrial | | | | | | | | |
| | OS = Glycine max PE = 3 | | | | | | | | |
| | SV = 1 - | | | | | | | | |
| | [I1MLQ8_SOYBN] | | | | | | | | |
| A0A072VAA5 | Succinate-semialdehyde | 8.24 | 7 | 4 | 4 | 4 | 522 | 55.8 | 6.92 |
| | dehydrogenase | | | | | | | | |
| | OS = Medicago truncatula | | | | | | | | |
| | GN = MTR_2g067660 | | | | | | | | |
| | PE = 3 SV = 1 - | | | | | | | | |
| | [A0A072VAA5_MEDTR] | | | | | | | | |
| A0A072VL01 | Succinyl-CoA ligase | 3.80 | 9 | 2 | 2 | 2 | 421 | 45.1 | 5.91 |
| | subunit beta | | | | | | | | |
| | OS = Medicago truncatula | | | | | | | | |
| | GN = MTR_1g069645 | | | | | | | | |
| | PE = 3 SV = 1 - | | | | | | | | |
| | [A0A072VL01_MEDTR] | | | | | | | | |
| A0A0B2P8I5 | Sulfite reductase | 6.55 | 9 | 3 | 3 | 3 | 687 | 77.2 | 9.16 |
| | [ferredoxin] OS = Glycine | | | | | | | | |
| | soja GN = glysoja_020481 | | | | | | | | |
| | PE = 4 SV = 1 - | | | | | | | | |
| | [A0A0B2P8I5_GLYSO] | | | | | | | | |
| Q75NZ0 | Sulfite reductase | 1.17 | 1 | 1 | 1 | 1 | 685 | 76.9 | 9.03 |
| | [ferredoxin], chloroplastic | | | | | | | | |
| | OS = Pisum sativum | | | | | | | | |
| | GN = SIR PE = 1 SV = 2 - | | | | | | | | |
| | [SIR_PEA] | | | | | | | | |
| V7BVY7 | Surfurtransferase | 2.64 | 1 | 1 | 1 | 1 | 379 | 41.9 | 6.95 |
| | OS = Phaseolus vulgaris | | | | | | | | |
| | GN = PHAVU_005G050400g | | | | | | | | |
| | PE = 4 SV = 1 - | | | | | | | | |
| | [V7BVY7_PHAVU] | | | | | | | | |
| C6T5D0 | Superoxide dismutase | 3.33 | 3 | 1 | 1 | 1 | 240 | 26.6 | 8.66 |
| | (Fragment) OS = Glycine | | | | | | | | |
| | max PE = 2 SV = 1 - | | | | | | | | |
| | [C6T5D0_SOYBN] | | | | | | | | |
| C6TFH5 | Superoxide dismutase | 11.63 | 9 | 1 | 1 | 1 | 129 | 13.7 | 6.37 |
| | [Cu—Zn] OS = Glycine max | | | | | | | | |
| | PE = 2 SV = 1 - | | | | | | | | |
| | [C6TFH5_SOYBN] | | | | | | | | |
| I3SQS9 | T-complex protein 1 | 14.56 | 16 | 2 | 2 | 2 | 158 | 17.1 | 7.31 |
| | subunit delta | | | | | | | | |
| | OS = Medicago truncatula | | | | | | | | |
| | PE = 2 SV = 1 - | | | | | | | | |
| | [I3SQS9_MEDTR] | | | | | | | | |
| A0A0B2S2R2 | T-complex protein 1 | 2.73 | 6 | 1 | 1 | 1 | 513 | 55.3 | 6.38 |
| | subunit eta OS = Glycine | | | | | | | | |
| | soja GN = glysoja_047968 | | | | | | | | |
| | PE = 3 SV = 1 - | | | | | | | | |
| | [A0A0B2S2R2_GLYSO] | | | | | | | | |
| A0A0B2P712 | T-complex protein 1 | 6.25 | 12 | 1 | 3 | 3 | 480 | 52.5 | 5.57 |
| | subunit gamma | | | | | | | | |
| | OS = Glycine soja | | | | | | | | |
| | GN = glysoja_013923 PE = 3 | | | | | | | | |
| | SV = 1 - | | | | | | | | |
| | [A0A0B2P712_GLYSO] | | | | | | | | |
| A0A0B2NX08 | T-complex protein 1 | 5.78 | 11 | 1 | 3 | 3 | 554 | 60.2 | 6.33 |
| | subunit gamma | | | | | | | | |
| | OS = Glycine soja | | | | | | | | |
| | GN = glysoja_023929 PE = 3 | | | | | | | | |
| | SV = 1 - | | | | | | | | |
| | [A0A0B2NX08_GLYSO] | | | | | | | | |
| G7J2L7 | TCP-1/cpn60 chaperonin | 3.93 | 7 | 2 | 2 | 2 | 535 | 59.2 | 5.66 |
| | family protein | | | | | | | | |
| | OS = Medicago truncatula | | | | | | | | |
| | GN = MTR_3g086330 | | | | | | | | |
| | PE = 2 SV = 1 - | | | | | | | | |
| | [G7J2L7_MEDTR] | | | | | | | | |
| Q38JD2 | Temperature-induced | 5.43 | 3 | 1 | 1 | 1 | 184 | 21.4 | 7.24 |
| | lipocalin OS = Glycine max | | | | | | | | |
| | GN = TIL PE = 2 SV = 1 - | | | | | | | | |
| | [Q38JD2_SOYBN] | | | | | | | | |
| Q06H32 | Thioredoxin fold | 54.32 | 14 | 6 | 6 | 7 | 162 | 17.4 | 5.86 |
| | OS = Arachis hypogaea | | | | | | | | |
| | PE = 2 SV = 1 - | | | | | | | | |
| | [Q06H32_ARAHY] | | | | | | | | |
| F8S297 | Thioredoxin h 1-1 | 27.50 | 2 | 2 | 2 | 3 | 120 | 13.1 | 5.85 |
| | OS = Galega orientalis | | | | | | | | |
| | GN = Trxh1-1 PE = 2 SV = 1 - | | | | | | | | |
| | [F8S297_9FABA] | | | | | | | | |
| A0A024NRI7 | Tonoplast intrinsic protein | 8.14 | 1 | 1 | 1 | 1 | 172 | 18.2 | 7.12 |
| | 32 (Fragment) OS = Vicia | | | | | | | | |
| | faba var. minor | | | | | | | | |
| | GN = TIP3; 2 PE = 2 SV = 1 - | | | | | | | | |
| | [A0A024NRI7_VICFA] | | | | | | | | |
| Q06H34 | Tonoplast intrinsic protein | 6.01 | 1 | 1 | 1 | 2 | 183 | 19.2 | 8.54 |
| | alpha TIP (Fragment) | | | | | | | | |
| | OS = Arachis hypogaea | | | | | | | | |
| | PE = 2 SV = 1 - | | | | | | | | |
| | [Q06H34_ARAHY] | | | | | | | | |
| A0A0K1JSF8 | Trans-cinnamate | 11.68 | 33 | 3 | 6 | 7 | 505 | 57.8 | 8.98 |
| | 4-monooxygenase | | | | | | | | |
| | OS = Oxytropis | | | | | | | | |
| | ochrocephala PE = 2 SV = 1 - | | | | | | | | |
| | [A0A0K1JSF8_9FABA] | | | | | | | | |
| A0A072UTU2 | Translation elongation | 8.59 | 25 | 2 | 3 | 3 | 384 | 44.1 | 6.14 |
| | factor EF1B, gamma chain | | | | | | | | |
| | OS = Medicago truncatula | | | | | | | | |
| | GN = MTR_4g134770 | | | | | | | | |
| | PE = 4 SV = 1 - | | | | | | | | |
| | [A0A072UTU2_MEDTR] | | | | | | | | |
| Q06H31 | Translationally controlled | 28.57 | 16 | 4 | 4 | 5 | 168 | 19.1 | 4.67 |
| | tumor-like protein | | | | | | | | |
| | OS = Arachis hypogaea | | | | | | | | |
| | PE = 2 SV = 1 - | | | | | | | | |
| | [Q06H31_ARAHY] | | | | | | | | |
| A0A0B2RK52 | Translocon-associated | 3.88 | 4 | 1 | 1 | 1 | 258 | 27.7 | 4.91 |
| | protein subunit alpha | | | | | | | | |
| | OS = Glycine soja | | | | | | | | |
| | GN = glysoja_039171 PE = 4 | | | | | | | | |
| | SV = 1 - | | | | | | | | |
| | [A0A0B2RK52_GLYSO] | | | | | | | | |
| C6TKJ5 | Triosephosphate isomerase | 17.86 | 14 | 1 | 4 | 6 | 252 | 27.1 | 6.55 |
| | OS = Glycine max PE = 2 | | | | | | | | |
| | SV = 1 - | | | | | | | | |
| | [C6TKJ5_SOYBN] | | | | | | | | |
| I1NAD0 | Triosephosphate isomerase | 7.52 | 8 | 1 | 2 | 4 | 306 | 33.0 | 7.68 |
| | OS = Glycine max PE = 3 | | | | | | | | |
| | SV = 1 - | | | | | | | | |
| | [I1NAD0_SOYBN] | | | | | | | | |
| A0A0B2SUS0 | Triosephosphate isomerase | 14.10 | 10 | 2 | 3 | 5 | 305 | 32.6 | 7.02 |
| | OS = Glycine soja | | | | | | | | |
| | GN = glysoja_007908 PE = 3 | | | | | | | | |
| | SV = 1 - | | | | | | | | |
| | [A0A0B2SUS0_GLYSO] | | | | | | | | |
| V7BWK1 | Triosephosphate isomerase | 15.81 | 11 | 1 | 4 | 6 | 253 | 27.1 | 6.23 |
| | OS = Phaseolus vulgaris | | | | | | | | |
| | GN = PHAVU_005G144100g | | | | | | | | |
| | PE = 3 SV = 1 - | | | | | | | | |
| | [V7BWK1_PHAVU] | | | | | | | | |
| A0A0B2S8E2 | Tripeptidyl-peptidase 2 | 4.68 | 9 | 4 | 6 | 7 | 1325 | 146.6 | 6.16 |
| | OS = Glycine soja | | | | | | | | |
| | GN = glysoja_036269 PE = 4 | | | | | | | | |
| | SV = 1 - | | | | | | | | |
| | [A0A0B2S8E2_GLYSO] | | | | | | | | |
| P01071 | Trypsin inhibitor B | 24.86 | 15 | 2 | 2 | 3 | 181 | 20.0 | 4.82 |
| | OS = Glycine max PE = 1 | | | | | | | | |
| | SV = 1 - [ITRB_SOYBN] | | | | | | | | |
| P28551 | Tubulin beta chain | 17.16 | 85 | 2 | 5 | 5 | 408 | 45.7 | 5.96 |
| | (Fragment) OS = Glycine | | | | | | | | |
| | max GN = TUBB PE = 2 | | | | | | | | |
| | SV = 2 - [TBB3_SOYBN] | | | | | | | | |
| Q0Q0Q9 | Type 4 metallothionein | 13.41 | 1 | 1 | 1 | 1 | 82 | 8.1 | 7.59 |
| | OS = Arachis hypogaea | | | | | | | | |
| | GN = MT4a PE = 2 SV = 1 - | | | | | | | | |
| | [Q0Q0Q9_ARAHY] | | | | | | | | |
| Q0Q0Q8 | Type 4 metallothionein | 51.22 | 1 | 2 | 2 | 2 | 82 | 8.2 | 7.59 |
| | OS = Arachis hypogaea | | | | | | | | |
| | GN = MT4b PE = 2 SV = 1 - | | | | | | | | |
| | [Q0Q0Q8_ARAHY] | | | | | | | | |
| O24548 | Type IIIa membrane | 9.25 | 36 | 3 | 3 | 3 | 346 | 39.4 | 6.67 |
| | protein cp-wap13 | | | | | | | | |
| | OS = Vigna unguiculata | | | | | | | | |
| | PE = 2 SV = 1 - | | | | | | | | |
| | [O24548_VIGUN] | | | | | | | | |
| O65733 | Ubiquitin conjugating | 18.03 | 58 | 1 | 1 | 1 | 61 | 6.9 | 7.18 |
| | enzyme (Fragment) | | | | | | | | |
| | OS = Cicer arietinum PE = 2 | | | | | | | | |
| | SV = 1 - [O65733_CICAR] | | | | | | | | |
| A0A0B2QSA3 | Ubiquitin-40S ribosomal | 23.18 | 102 | 4 | 4 | 8 | 151 | 17.2 | 9.83 |
| | protein S27a OS = Glycine | | | | | | | | |
| | soja GN = glysoja_030669 | | | | | | | | |
| | PE = 4 SV = 1 - | | | | | | | | |
| | [A0A0B2QSA3_GLYSO] | | | | | | | | |
| A0A072TUI6 | Ubiquitin-conjugating | 13.89 | 31 | 1 | 1 | 1 | 72 | 8.6 | 5.73 |
| | enzyme OS = Medicago | | | | | | | | |
| | truncatula | | | | | | | | |
| | GN = MTR_8g098615 | | | | | | | | |
| | PE = 4 SV = 1 - | | | | | | | | |
| | [A0A072TUI6_MEDTR] | | | | | | | | |
| A0A0K0L2S7 | UDP-glucose | 47.83 | 1 | 1 | 2 | 4 | 46 | 5.1 | 4.55 |
| | pyrophosphorylase | | | | | | | | |
| | (Fragment) OS = Pisum | | | | | | | | |
| | sativum GN = UGP PE = 4 | | | | | | | | |
| | SV = 1 - | | | | | | | | |
| | [A0A0K0L2S7_PEA] | | | | | | | | |
| Q8W557 | UDP-glucose | 25.69 | 5 | 1 | 10 | 13 | 471 | 51.6 | 6.48 |
| | pyrophosphorylase | | | | | | | | |
| | OS = Amorpha fruticosa | | | | | | | | |
| | GN = ugp PE = 2 SV = 1 - | | | | | | | | |
| | [Q8W557_9FABA] | | | | | | | | |
| I1K0A0 | Uncharacterized protein | 3.35 | 11 | 1 | 1 | 1 | 418 | 46.1 | 5.94 |
| | (Fragment) OS = Glycine | | | | | | | | |
| | max PE = 3 SV = 1 - | | | | | | | | |
| | [I1K0A0_SOYBN] | | | | | | | | |
| K7N4A7 | Uncharacterized protein | 3.92 | 8 | 1 | 1 | 1 | 255 | 27.6 | 6.05 |
| | (Fragment) OS = Glycine | | | | | | | | |
| | max PE = 3 SV = 1 - | | | | | | | | |
| | [K7N4A7_SOYBN] | | | | | | | | |
| K7K2B4 | Uncharacterized protein | 11.22 | 23 | 1 | 1 | 1 | 98 | 11.3 | 5.94 |
| | (Fragment) OS = Glycine | | | | | | | | |
| | max PE = 4 SV = 1 - | | | | | | | | |
| | [K7K2B4_SOYBN] | | | | | | | | |
| K7KQF1 | Uncharacterized protein | 3.67 | 20 | 1 | 1 | 1 | 218 | 25.2 | 7.53 |
| | (Fragment) OS = Glycine | | | | | | | | |
| | max PE = 4 SV = 1 - | | | | | | | | |
| | [K7KQF1_SOYBN] | | | | | | | | |
| K7L981 | Uncharacterized protein | 14.29 | 22 | 1 | 1 | 1 | 84 | 10.0 | 10.92 |
| | (Fragment) OS = Glycine | | | | | | | | |
| | max PE = 4 SV = 1 - | | | | | | | | |
| | [K7L981_SOYBN] | | | | | | | | |
| K7LHL1 | Uncharacterized protein | 17.53 | 10 | 1 | 1 | 1 | 97 | 11.0 | 9.19 |
| | (Fragment) OS = Glycine | | | | | | | | |
| | max PE = 4 SV = 1 - | | | | | | | | |
| | [K7LHL1_SOYBN] | | | | | | | | |
| K7LWW6 | Uncharacterized protein | 7.53 | 6 | 1 | 1 | 1 | 146 | 16.8 | 7.05 |
| | (Fragment) OS = Glycine | | | | | | | | |
| | max PE = 4 SV = 1 - | | | | | | | | |
| | [K7LWW6_SOYBN] | | | | | | | | |
| A0A0L9TPW7 | Uncharacterized protein | 3.47 | 10 | 1 | 1 | 1 | 346 | 38.0 | 5.67 |
| | (Fragment) OS = Phaseolus | | | | | | | | |
| | angularis | | | | | | | | |
| | GN = LR48_Vigan01g216100 | | | | | | | | |
| | PE = 4 SV = 1 - | | | | | | | | |
| | [A0A0L9TPW7_PHAAN] | | | | | | | | |
| A0A0L9TV69 | Uncharacterized protein | 24.44 | 9 | 1 | 11 | 11 | 577 | 61.1 | 5.66 |
| | (Fragment) OS = Phaseolus | | | | | | | | |
| | angularis | | | | | | | | |
| | GN = LR48_Vigan02g055100 | | | | | | | | |
| | PE = 4 SV = 1 - | | | | | | | | |
| | [A0A0L9TV69_PHAAN] | | | | | | | | |
| A0A0L9TYM3 | Uncharacterized protein | 16.88 | 15 | 1 | 1 | 1 | 77 | 9.1 | 11.17 |
| | (Fragment) OS = Phaseolus | | | | | | | | |
| | angularis | | | | | | | | |
| | GN = LR48_Vigan02g143400 | | | | | | | | |
| | PE = 4 SV = 1 - | | | | | | | | |
| | [A0A0L9TYM3_PHAAN] | | | | | | | | |
| A0A0L9U8N2 | Uncharacterized protein | 27.92 | 19 | 3 | 3 | 4 | 197 | 22.0 | 4.98 |
| | (Fragment) OS = Phaseolus | | | | | | | | |
| | angularis | | | | | | | | |
| | GN = LR48_Vigan03g247300 | | | | | | | | |
| | PE = 4 SV = 1 - | | | | | | | | |
| | [A0A0L9U8N2_PHAAN] | | | | | | | | |
| A0A0L9USQ9 | Uncharacterized protein | 2.76 | 9 | 1 | 2 | 2 | 870 | 97.2 | 6.73 |
| | (Fragment) OS = Phaseolus | | | | | | | | |
| | angularis | | | | | | | | |
| | GN = LR48_Vigan06g109400 | | | | | | | | |
| | PE = 4 SV = 1 - | | | | | | | | |
| | [A0A0L9USQ9_PHAAN] | | | | | | | | |
| A0A0L9V7A7 | Uncharacterized protein | 4.64 | 10 | 1 | 1 | 1 | 302 | 33.4 | 5.36 |
| | (Fragment) OS = Phaseolus | | | | | | | | |
| | angularis | | | | | | | | |
| | GN = LR48_Vigan08g137100 | | | | | | | | |
| | PE = 4 SV = 1 - | | | | | | | | |
| | [A0A0L9V7A7_PHAAN] | | | | | | | | |
| A0A0L9V7B4 | Uncharacterized protein | 5.56 | 3 | 1 | 4 | 4 | 791 | 89.1 | 5.57 |
| | (Fragment) OS = Phaseolus | | | | | | | | |
| | angularis | | | | | | | | |
| | GN = LR48_Vigan08g138600 | | | | | | | | |
| | PE = 4 SV = 1 - | | | | | | | | |
| | [A0A0L9V7B4_PHAAN] | | | | | | | | |
| A0A0L9T3U5 | Uncharacterized protein | 17.65 | 4 | 1 | 1 | 1 | 102 | 11.7 | 10.64 |
| | (Fragment) OS = Phaseolus | | | | | | | | |
| | angularis | | | | | | | | |
| | GN = LR48_Vigan45s000800 | | | | | | | | |
| | PE = 4 SV = 1 - | | | | | | | | |
| | [A0A0L9T3U5_PHAAN] | | | | | | | | |
| A0A0L9TC30 | Uncharacterized protein | 4.11 | 10 | 1 | 3 | 3 | 900 | 98.3 | 6.30 |
| | (Fragment) OS = Phaseolus | | | | | | | | |
| | angularis | | | | | | | | |
| | GN = LR48_Vigan499s005200 | | | | | | | | |
| | PE = 4 SV = 1 - | | | | | | | | |
| | [A0A0L9TC30_PHAAN] | | | | | | | | |
| V7C2J7 | Uncharacterized protein | 3.81 | 3 | 1 | 1 | 1 | 236 | 26.3 | 7.02 |
| | (Fragment) OS = Phaseolus | | | | | | | | |
| | vulgaris | | | | | | | | |
| | GN = PHAVU_004G0542000g | | | | | | | | |
| | PE = 4 SV = 1 - | | | | | | | | |
| | [V7C2J7_PHAVU] | | | | | | | | |
| C6SV94 | Uncharacterized protein | 22.67 | 11 | 3 | 3 | 3 | 150 | 16.3 | 10.87 |
| | OS = Glycine max PE = 2 | | | | | | | | |
| | SV = 1 - | | | | | | | | |
| | [C6SV94_SOYBN] | | | | | | | | |
| C6SVD6 | Uncharacterized protein | 16.46 | 6 | 1 | 1 | 1 | 79 | 8.8 | 8.76 |
| | OS = Glycine max PE = 2 | | | | | | | | |
| | SV = 1 - | | | | | | | | |
| | [C6SVD6_SOYBN] | | | | | | | | |
| C6SWQ6 | Uncharacterized protein | 16.33 | 8 | 3 | 3 | 3 | 245 | 26.9 | 9.52 |
| | OS = Glycine max PE = 2 | | | | | | | | |
| | SV = 1 - | | | | | | | | |
| | [C6SWQ6_SOYBN] | | | | | | | | |
| C6SZQ3 | Uncharacterized protein | 32.45 | 27 | 5 | 5 | 6 | 151 | 17.2 | 10.21 |
| | OS = Glycine max PE = 2 | | | | | | | | |
| | SV = 1 - | | | | | | | | |
| | [C6SZQ3_SOYBN] | | | | | | | | |
| C6T2B7 | Uncharacterized protein | 11.83 | 5 | 1 | 1 | 1 | 93 | 10.3 | 5.82 |
| | OS = Glycine max PE = 2 | | | | | | | | |
| | SV = 1 - | | | | | | | | |
| | [C6T2B7_SOYBN] | | | | | | | | |
| C6T551 | Uncharacterized protein | 4.58 | 3 | 1 | 1 | 1 | 153 | 17.3 | 5.99 |
| | OS = Glycine max PE = 2 | | | | | | | | |
| | SV = 1 - | | | | | | | | |
| | [C6T551_SOYBN] | | | | | | | | |
| C6TA47 | Uncharacterized protein | 18.75 | 7 | 1 | 1 | 1 | 112 | 12.5 | 5.25 |
| | OS = Glycine max PE = 2 | | | | | | | | |
| | SV = 1 - | | | | | | | | |
| | [C6TA47_SOYBN] | | | | | | | | |
| C6TAY3 | Uncharacterized protein | 10.38 | 19 | 2 | 2 | 2 | 289 | 31.8 | 9.42 |
| | OS = Glycine max PE = 2 | | | | | | | | |
| | SV = 1 - | | | | | | | | |
| | [C6TAY3_SOYBN] | | | | | | | | |
| C6TGA6 | Uncharacterized protein | 6.58 | 21 | 2 | 2 | 2 | 319 | 34.2 | 5.11 |
| | OS = Glycine max PE = 2 | | | | | | | | |
| | SV = 1 - | | | | | | | | |
| | [C6TGA6_SOYBN] | | | | | | | | |
| C6TID4 | Uncharacterized protein | 5.85 | 4 | 2 | 2 | 2 | 325 | 34.5 | 6.24 |
| | OS = Glycine max PE = 2 | | | | | | | | |
| | SV = 1 - | | | | | | | | |
| | [C6TID4_SOYBN] | | | | | | | | |
| C6TKH0 | Uncharacterized protein | 8.16 | 4 | 2 | 2 | 2 | 294 | 31.6 | 6.84 |
| | OS = Glycine max PE = 2 | | | | | | | | |
| | SV = 1 - | | | | | | | | |
| | [C6TKH0_SOYBN] | | | | | | | | |
| I1JC08 | Uncharacterized protein | 1.55 | 2 | 1 | 1 | 1 | 710 | 81.8 | 7.21 |
| | OS = Glycine max PE = 3 | | | | | | | | |
| | SV = 1 - [I1JC08_SOYBN] | | | | | | | | |
| I1JFW7 | Uncharacterized protein | 21.54 | 27 | 4 | 4 | 5 | 195 | 21.8 | 9.29 |
| | OS = Glycine max PE = 3 | | | | | | | | |
| | SV = 1 - | | | | | | | | |
| | [I1JFW7_SOYBN] | | | | | | | | |
| I1JPW5 | Uncharacterized protein | 30.63 | 11 | 4 | 8 | 22 | 444 | 47.6 | 5.69 |
| | OS = Glycine max PE = 3 | | | | | | | | |
| | SV = 1 - | | | | | | | | |
| | [I1JPW5_SOYBN] | | | | | | | | |
| I1JWW7 | Uncharacterized protein | 9.29 | 13 | 1 | 2 | 2 | 183 | 21.5 | 10.21 |
| | OS = Glycine max PE = 3 | | | | | | | | |
| | SV = 1 - | | | | | | | | |
| | [I1JWW7_SOYBN] | | | | | | | | |
| I1KZJ1 | Uncharacterized protein | 7.65 | 14 | 3 | 3 | 5 | 392 | 43.0 | 5.54 |
| | OS = Glycine max PE = 3 | | | | | | | | |
| | SV = 1 - [I1KZJ1_SOYBN] | | | | | | | | |
| I1LQD7 | Uncharacterized protein | 22.73 | 41 | 2 | 6 | 6 | 330 | 35.4 | 5.26 |
| | OS = Glycine max PE = 3 | | | | | | | | |
| | SV = 1 - | | | | | | | | |
| | [I1LQD7_SOYBN] | | | | | | | | |
| I1LUM3 | Uncharacterized protein | 10.00 | 61 | 1 | 3 | 4 | 260 | 29.3 | 4.82 |
| | OS = Glycine max PE = 3 | | | | | | | | |
| | SV = 1 - | | | | | | | | |
| | [I1LUM3_SOYBN] | | | | | | | | |
| I1LXD1 | Uncharacterized protein | 10.64 | 47 | 2 | 3 | 7 | 357 | 38.7 | 7.94 |
| | OS = Glycine max PE = 3 | | | | | | | | |
| | SV = 1 - | | | | | | | | |
| | [I1LXD1_SOYBN] | | | | | | | | |
| I1MM85 | Uncharacterized protein | 8.41 | 9 | 1 | 4 | 4 | 523 | 58.5 | 6.73 |
| | OS = Glycine max PE = 3 | | | | | | | | |
| | SV = 1 - | | | | | | | | |
| | [I1MM85_SOYBN] | | | | | | | | |
| I1MT10 | Uncharacterized protein | 22.95 | 53 | 0 | 11 | 16 | 645 | 70.8 | 5.39 |
| | OS = Glycine max PE = 3 | | | | | | | | |
| | SV = 1 - | | | | | | | | |
| | [I1MT10_SOYBN] | | | | | | | | |
| I1NJ15 | Uncharacterized protein | 12.68 | 44 | 2 | 3 | 3 | 355 | 39.8 | 4.98 |
| | OS = Glycine max PE = 3 | | | | | | | | |
| | SV = 1 - [I1NJ15_SOYBN] | | | | | | | | |
| K7KFI7 | Uncharacterized protein | 12.00 | 4 | 1 | 1 | 1 | 125 | 13.4 | 10.01 |
| | OS = Glycine max PE = 3 | | | | | | | | |
| | SV = 1 - | | | | | | | | |
| | [K7KFI7_SOYBN] | | | | | | | | |
| K7L593 | Uncharacterized protein | 16.48 | 9 | 1 | 7 | 8 | 540 | 57.8 | 5.19 |
| | OS = Glycine max PE = 3 | | | | | | | | |
| | SV = 1 - | | | | | | | | |
| | [K7L593_SOYBN] | | | | | | | | |
| K7L5A8 | Uncharacterized protein | 2.95 | 5 | 1 | 1 | 1 | 440 | 47.4 | 7.20 |
| | OS = Glycine max PE = 3 | | | | | | | | |
| | SV = 1 - | | | | | | | | |
| | [K7L5A8_SOYBN] | | | | | | | | |
| K7LPV4 | Uncharacterized protein | 15.45 | 23 | 2 | 2 | 2 | 123 | 13.9 | 10.52 |
| | OS = Glycine max PE = 3 | | | | | | | | |
| | SV = 1 - | | | | | | | | |
| | [K7LPV4_SOYBN] | | | | | | | | |
| K7MTK8 | Uncharacterized protein | 4.18 | 7 | 1 | 1 | 1 | 263 | 29.1 | 8.97 |
| | OS = Glycine max PE = 3 | | | | | | | | |
| | SV = 1 - | | | | | | | | |
| | [K7MTK8_SOYBN] | | | | | | | | |
| K7MVD3 | Uncharacterized protein | 23.65 | 80 | 1 | 13 | 20 | 647 | 70.8 | 5.21 |
| | OS = Glycine max PE = 3 | | | | | | | | |
| | SV = 1 - | | | | | | | | |
| | [K7MVD3_SOYBN] | | | | | | | | |
| I1MS56 | Uncharacterized protein | 4.57 | 7 | 1 | 1 | 1 | 197 | 20.7 | 6.00 |
| | OS = Glycine max PE = 3 | | | | | | | | |
| | SV = 2 - | | | | | | | | |
| | [I1MS56_SOYBN] | | | | | | | | |
| I1JMY4 | Uncharacterized protein | 1.78 | 1 | 1 | 1 | 1 | 449 | 50.4 | 7.78 |
| | OS = Glycine max PE = 4 | | | | | | | | |
| | SV = 1 - | | | | | | | | |
| | [I1JMY4_SOYBN] | | | | | | | | |
| I1JQN0 | Uncharacterized protein | 30.68 | 33 | 5 | 5 | 5 | 176 | 19.4 | 11.43 |
| | OS = Glycine max PE = 4 | | | | | | | | |
| | SV = 1 - [I1JQN0_SOYBN] | | | | | | | | |
| I1JYG3 | Uncharacterized protein | 22.92 | 21 | 1 | 4 | 4 | 192 | 21.7 | 9.50 |
| | OS = Glycine max PE = 4 | | | | | | | | |
| | SV = 1 - [I1JYG3_SOYBN] | | | | | | | | |
| I1KA65 | Uncharacterized protein | 20.27 | 7 | 1 | 1 | 1 | 74 | 7.9 | 8.76 |
| | OS = Glycine max PE = 4 | | | | | | | | |
| | SV = 1 - | | | | | | | | |
| | [I1KA65_SOYBN] | | | | | | | | |
| I1KBE4 | Uncharacterized protein | 5.42 | 3 | 1 | 1 | 1 | 240 | 26.8 | 10.20 |
| | OS = Glycine max PE = 4 | | | | | | | | |
| | SV = 1 - | | | | | | | | |
| | [I1KBE4_SOYBN] | | | | | | | | |
| I1KHK3 | Uncharacterized protein | 7.25 | 20 | 2 | 2 | 2 | 331 | 36.6 | 5.20 |
| | OS = Glycine max PE = 4 | | | | | | | | |
| | SV = 1 - | | | | | | | | |
| | [I1KHK3_SOYBN] | | | | | | | | |
| I1KKK1 | Uncharacterized protein | 4.87 | 11 | 1 | 1 | 1 | 226 | 24.4 | 6.19 |
| | OS = Glycine max PE = 4 | | | | | | | | |
| | SV = 1 - | | | | | | | | |
| | [I1KKK1_SOYBN] | | | | | | | | |
| I1KNE0 | Uncharacterized protein | 6.82 | 28 | 1 | 2 | 3 | 264 | 29.8 | 5.40 |
| | OS = Glycine max PE = 4 | | | | | | | | |
| | SV = 1 - | | | | | | | | |
| | [I1KNE0_SOYBN] | | | | | | | | |
| I1L0Q8 | Uncharacterized protein | 2.18 | 13 | 1 | 1 | 1 | 458 | 49.0 | 6.84 |
| | OS = Glycine max PE = 4 | | | | | | | | |
| | SV = 1 - [I1L0Q8_SOYBN] | | | | | | | | |
| I1L862 | Uncharacterized protein | 10.65 | 8 | 3 | 3 | 19 | 460 | 52.9 | 6.87 |
| | OS = Glycine max PE = 4 | | | | | | | | |
| | SV = 1 - [I1L862_SOYBN] | | | | | | | | |
| I1LDB3 | Uncharacterized protein | 12.96 | 54 | 2 | 2 | 2 | 162 | 18.1 | 4.79 |
| | OS = Glycine max PE = 4 | | | | | | | | |
| | SV = 1 - | | | | | | | | |
| | [I1LDB3_SOYBN] | | | | | | | | |
| I1LM73 | Uncharacterized protein | 26.11 | 7 | 1 | 10 | 13 | 475 | 51.9 | 5.49 |
| | OS = Glycine max PE = 4 | | | | | | | | |
| | SV = 1 - | | | | | | | | |
| | [I1LM73_SOYBN] | | | | | | | | |
| I1LQ07 | Uncharacterized protein | 4.10 | 14 | 1 | 2 | 2 | 512 | 56.3 | 5.49 |
| | OS = Glycine max PE = 4 | | | | | | | | |
| | SV = 1 - [I1LQ07_SOYBN] | | | | | | | | |
| I1LX29 | Uncharacterized protein | 5.29 | 7 | 1 | 1 | 1 | 170 | 19.8 | 9.95 |
| | OS = Glycine max PE = 4 | | | | | | | | |
| | SV = 1 - [I1LX29_SOYBN] | | | | | | | | |
| I1MGG5 | Uncharacterized protein | 14.07 | 9 | 2 | 2 | 2 | 135 | 14.6 | 7.14 |
| | OS = Glycine max PE = 4 | | | | | | | | |
| | SV = 1 - | | | | | | | | |
| | [I1MGG5_SOYBN] | | | | | | | | |
| I1MN82 | Uncharacterized protein | 5.53 | 11 | 3 | 3 | 3 | 488 | 55.3 | 5.87 |
| | OS = Glycine max PE = 4 | | | | | | | | |
| | SV = 1 - | | | | | | | | |
| | [I1MN82_SOYBN] | | | | | | | | |
| I1MR18 | Uncharacterized protein | 5.42 | 4 | 1 | 1 | 1 | 203 | 21.3 | 7.14 |
| | OS = Glycine max PE = 4 | | | | | | | | |
| | SV = 1 - | | | | | | | | |
| | [I1MR18_SOYBN] | | | | | | | | |
| I1NGH2 | Uncharacterized protein | 15.13 | 11 | 3 | 5 | 64 | 542 | 63.2 | 5.05 |
| | OS = Glycine max PE = 4 | | | | | | | | |
| | SV = 1 - | | | | | | | | |
| | [I1NGH2_SOYBN] | | | | | | | | |
| I1NI77 | Uncharacterized protein | 25.19 | 26 | 4 | 4 | 4 | 131 | 15.3 | 10.39 |
| | OS = Glycine max PE = 4 | | | | | | | | |
| | SV = 1 - [I1NI77_SOYBN] | | | | | | | | |
| I1NJC3 | Uncharacterized protein | 2.01 | 6 | 1 | 1 | 1 | 447 | 50.3 | 6.21 |
| | OS = Glycine max PE = 4 | | | | | | | | |
| | SV = 1 - [I1NJC3_SOYBN] | | | | | | | | |
| K7KDU5 | Uncharacterized protein | 12.22 | 8 | 1 | 1 | 1 | 90 | 9.9 | 8.44 |
| | OS = Glycine max PE = 4 | | | | | | | | |
| | SV = 1 - | | | | | | | | |
| | [K7KDU5_SOYBN] | | | | | | | | |
| K7KF08 | Uncharacterized protein | 14.12 | 19 | 1 | 1 | 1 | 85 | 9.6 | 7.50 |
| | OS = Glycine max PE = 4 | | | | | | | | |
| | SV = 1 - | | | | | | | | |
| | [K7KF08_SOYBN] | | | | | | | | |
| K7KSG8 | Uncharacterized protein | 15.58 | 15 | 2 | 2 | 2 | 154 | 14.8 | 9.54 |
| | OS = Glycine max PE = 4 | | | | | | | | |
| | SV = 1 - | | | | | | | | |
| | [K7KSG8_SOYBN] | | | | | | | | |
| K7KZX8 | Uncharacterized protein | 10.33 | 19 | 1 | 2 | 3 | 184 | 20.8 | 6.10 |
| | OS = Glycine max PE = 4 | | | | | | | | |
| | SV = 1 - | | | | | | | | |
| | [K7KZX8_SOYBN] | | | | | | | | |
| K7LHR7 | Uncharacterized protein | 12.68 | 12 | 1 | 1 | 1 | 71 | 7.6 | 7.85 |
| | OS = Glycine max PE = 4 | | | | | | | | |
| | SV = 1 - | | | | | | | | |
| | [K7LHR7_SOYBN] | | | | | | | | |
| K7LL21 | Uncharacterized protein | 2.60 | 3 | 1 | 3 | 3 | 1271 | 140.7 | 5.80 |
| | OS = Glycine max PE = 4 | | | | | | | | |
| | SV = 1 - | | | | | | | | |
| | [K7LL21_SOYBN] | | | | | | | | |
| K7LPJ2 | Uncharacterized protein | 6.73 | 7 | 2 | 2 | 2 | 401 | 45.6 | 6.29 |
| | OS = Glycine max PE = 4 | | | | | | | | |
| | SV = 1 - | | | | | | | | |
| | [K7LPJ2_SOYBN] | | | | | | | | |
| K7LPY2 | Uncharacterized protein | 3.45 | 14 | 1 | 1 | 1 | 290 | 31.8 | 8.12 |
| | OS = Glycine max PE = 4 | | | | | | | | |
| | SV = 1 - | | | | | | | | |
| | [K7LPY2_SOYBN] | | | | | | | | |
| K7LV71 | Uncharacterized protein | 22.35 | 24 | 3 | 3 | 3 | 170 | 19.0 | 5.21 |
| | OS = Glycine max PE = 4 | | | | | | | | |
| | SV = 1 - | | | | | | | | |
| | [K7LV71_SOYBN] | | | | | | | | |
| K7M3V1 | Uncharacterized protein | 1.82 | 2 | 1 | 1 | 1 | 384 | 43.9 | 4.75 |
| | OS = Glycine max PE = 4 | | | | | | | | |
| | SV = 1 - | | | | | | | | |
| | [K7M3V1_SOYBN] | | | | | | | | |
| K7MDM9 | Uncharacterized protein | 2.47 | 5 | 1 | 1 | 1 | 324 | 38.5 | 5.59 |
| | OS = Glycine max PE = 4 | | | | | | | | |
| | SV = 1 - | | | | | | | | |
| | [K7MDM9_SOYBN] | | | | | | | | |
| K7MPX7 | Uncharacterized protein | 4.86 | 4 | 1 | 1 | 1 | 329 | 34.9 | 5.48 |
| | OS = Glycine max PE = 4 | | | | | | | | |
| | SV = 1 - | | | | | | | | |
| | [K7MPX7_SOYBN] | | | | | | | | |
| K7MSK8 | Uncharacterized protein | 23.40 | 4 | 1 | 1 | 1 | 47 | 5.4 | 9.47 |
| | OS = Glycine max PE = 4 | | | | | | | | |
| | SV = 1 - | | | | | | | | |
| | [K7MSK8_SOYBN] | | | | | | | | |
| K7MT99 | Uncharacterized protein | 3.38 | 6 | 1 | 1 | 1 | 237 | 26.7 | 7.17 |
| | OS = Glycine max PE = 4 | | | | | | | | |
| | SV = 1 - | | | | | | | | |
| | [K7MT99_SOYBN] | | | | | | | | |
| K7MUE0 | Uncharacterized protein | 3.00 | 7 | 1 | 1 | 1 | 400 | 45.0 | 7.34 |
| | OS = Glycine max PE = 4 | | | | | | | | |
| | SV = 1 - | | | | | | | | |
| | [K7MUE0_SOYBN] | | | | | | | | |
| K7N2N0 | Uncharacterized protein | 23.40 | 156 | 2 | 2 | 2 | 94 | 10.4 | 8.69 |
| | OS = Glycine max PE = 4 | | | | | | | | |
| | SV = 1 - | | | | | | | | |
| | [K7N2N0_SOYBN] | | | | | | | | |
| K7N388 | Uncharacterized protein | 12.96 | 9 | 1 | 1 | 1 | 54 | 6.1 | 9.77 |
| | OS = Glycine max PE = 4 | | | | | | | | |
| | SV = 1 - | | | | | | | | |
| | [K7N388_SOYBN] | | | | | | | | |
| I1KN59 | Uncharacterized protein | 6.59 | 6 | 6 | 6 | 7 | 926 | 101.6 | 5.85 |
| | OS = Glycine max PE = 4 | | | | | | | | |
| | SV = 2 - | | | | | | | | |
| | [I1KN59_SOYBN] | | | | | | | | |
| A0A0B2RV11 | Uncharacterized protein | 4.81 | 6 | 1 | 1 | 1 | 270 | 29.2 | 6.96 |
| | OS = Glycine soja | | | | | | | | |
| | GN = glysoja_003212 PE = 4 | | | | | | | | |
| | SV = 1 - | | | | | | | | |
| | [A0A0B2RV11_GLYSO] | | | | | | | | |
| A0A0B2PQ70 | Uncharacterized protein | 4.71 | 5 | 1 | 1 | 1 | 170 | 18.5 | 6.00 |
| | OS = Glycine soja | | | | | | | | |
| | GN = glysoja_006014 PE = 4 | | | | | | | | |
| | SV = 1 - | | | | | | | | |
| | [A0A0B2PQ70_GLYSO] | | | | | | | | |
| A0A0B2RYE0 | Uncharacterized protein | 4.61 | 2 | 1 | 1 | 1 | 217 | 24.1 | 6.54 |
| | OS = Glycine soja | | | | | | | | |
| | GN = glysoja_016873 PE = 4 | | | | | | | | |
| | SV = 1 - | | | | | | | | |
| | [A0A0B2RYE0_GLYSO] | | | | | | | | |
| A0A0B2QJ33 | Uncharacterized protein | 11.54 | 5 | 1 | 1 | 1 | 78 | 8.2 | 4.97 |
| | OS = Glycine soja | | | | | | | | |
| | GN = glysoja_026551 PE = 4 | | | | | | | | |
| | SV = 1 - | | | | | | | | |
| | [A0A0B2QJ33_GLYSO] | | | | | | | | |
| A0A0B2RFD9 | Uncharacterized protein | 10.10 | 6 | 1 | 1 | 1 | 198 | 21.2 | 6.29 |
| | OS = Glycine soja | | | | | | | | |
| | GN = glysoja_034893 PE = 4 | | | | | | | | |
| | SV = 1 - | | | | | | | | |
| | [A0A0B2RFD9_GLYSO] | | | | | | | | |
| A0A0B2RAS1 | Uncharacterized protein | 3.98 | 3 | 1 | 1 | 1 | 427 | 47.1 | 6.60 |
| | OS = Glycine soja | | | | | | | | |
| | GN = glysoja_040216 PE = 4 | | | | | | | | |
| | SV = 1 - | | | | | | | | |
| | [A0A0B2RAS1_GLYSO] | | | | | | | | |
| A0A0B2SH08 | Uncharacterized protein | 15.38 | 2 | 1 | 1 | 1 | 91 | 10.1 | 4.91 |
| | OS = Glycine soja | | | | | | | | |
| | GN = glysoja_040939 PE = 4 | | | | | | | | |
| | SV = 1 - | | | | | | | | |
| | [A0A0B2SH08_GLYSO] | | | | | | | | |
| A0A0B2SEL9 | Uncharacterized protein | 15.91 | 22 | 1 | 2 | 2 | 176 | 19.3 | 4.73 |
| | OS = Glycine soja | | | | | | | | |
| | GN = glysoja_044134 PE = 4 | | | | | | | | |
| | SV = 1 - | | | | | | | | |
| | [A0A0B2SEL9_GLYSO] | | | | | | | | |
| A0A0B2P3H7 | Uncharacterized protein | 9.88 | 6 | 1 | 1 | 1 | 172 | 18.4 | 10.05 |
| | OS = Glycine soja | | | | | | | | |
| | GN = glysoja_046465 PE = 4 | | | | | | | | |
| | SV = 1 - | | | | | | | | |
| | [A0A0B2P3H7_GLYSO] | | | | | | | | |
| I3RZE7 | Uncharacterized protein | 3.56 | 10 | 1 | 1 | 1 | 225 | 24.9 | 9.28 |
| | OS = Lotus japonicus PE = 2 | | | | | | | | |
| | SV = 1 - [I3RZE7_LOTJA] | | | | | | | | |
| I3RZH0 | Uncharacterized protein | 16.92 | 14 | 1 | 1 | 1 | 65 | 6.9 | 9.35 |
| | OS = Lotus japonicus PE = 2 | | | | | | | | |
| | SV = 1 - [I3RZH0_LOTJA] | | | | | | | | |
| I3RZW5 | Uncharacterized protein | 7.66 | 2 | 1 | 2 | 2 | 274 | 32.1 | 5.07 |
| | OS = Lotus japonicus PE = 2 | | | | | | | | |
| | SV = 1 - | | | | | | | | |
| | [I3RZW5_LOTJA] | | | | | | | | |
| I3S105 | Uncharacterized protein | 6.83 | 1 | 1 | 1 | 1 | 161 | 17.7 | 6.62 |
| | OS = Lotus japonicus PE = 2 | | | | | | | | |
| | SV = 1 - [I3S105_LOTJA] | | | | | | | | |
| I3S161 | Uncharacterized protein | 7.59 | 39 | 1 | 1 | 2 | 145 | 15.7 | 10.07 |
| | OS = Lotus japonicus PE = 2 | | | | | | | | |
| | SV = 1 - [I3S161_LOTJA] | | | | | | | | |
| I3S1S2 | Uncharacterized protein | 13.59 | 3 | 1 | 2 | 3 | 206 | 22.6 | 4.49 |
| | OS = Lotus japonicus PE = 2 | | | | | | | | |
| | SV = 1 - [I3S1S2_LOTJA] | | | | | | | | |
| I3S2A3 | Uncharacterized protein | 5.98 | 9 | 1 | 2 | 2 | 301 | 34.4 | 9.33 |
| | OS = Lotus japonicus PE = 2 | | | | | | | | |
| | SV = 1 - [I3S2A3_LOTJA] | | | | | | | | |
| I3S3B1 | Uncharacterized protein | 3.31 | 8 | 1 | 1 | 1 | 453 | 49.4 | 6.62 |
| | OS = Lotus japonicus PE = 2 | | | | | | | | |
| | SV = 1 - [I3S3B1_LOTJA] | | | | | | | | |
| I3S3I9 | Uncharacterized protein | 9.02 | 12 | 1 | 1 | 1 | 133 | 15.2 | 6.95 |
| | OS = Lotus japonicus PE = 2 | | | | | | | | |
| | SV = 1 - [I3S3I9_LOTJA] | | | | | | | | |
| I3S3N5 | Uncharacterized protein | 6.71 | 13 | 1 | 1 | 1 | 164 | 18.7 | 10.08 |
| | OS = Lotus japonicus PE = 2 | | | | | | | | |
| | SV = 1 - [I3S3N5_LOTJA] | | | | | | | | |
| I3S3W2 | Uncharacterized protein | 4.28 | 14 | 1 | 1 | 1 | 187 | 21.2 | 6.81 |
| | OS = Lotus japonicus PE = 2 | | | | | | | | |
| | SV = 1 - [I3S3W2_LOTJA] | | | | | | | | |
| I3S408 | Uncharacterized protein | 26.44 | 3 | 2 | 2 | 2 | 87 | 9.7 | 8.13 |
| | OS = Lotus japonicus PE = 2 | | | | | | | | |
| | SV = 1 - [I3S408_LOTJA] | | | | | | | | |
| I3S4F8 | Uncharacterized protein | 14.67 | 3 | 1 | 2 | 2 | 150 | 16.9 | 10.90 |
| | OS = Lotus japonicus PE = 2 | | | | | | | | |
| | SV = 1 - [I3S4F8_LOTJA] | | | | | | | | |
| I3S6E2 | Uncharacterized protein | 26.88 | 55 | 6 | 6 | 6 | 346 | 38.9 | 7.56 |
| | OS = Lotus japonicus PE = 2 | | | | | | | | |
| | SV = 1 - [I3S6E2_LOTJA] | | | | | | | | |
| I3S764 | Uncharacterized protein | 7.95 | 11 | 1 | 2 | 2 | 302 | 34.5 | 9.09 |
| | OS = Lotus japonicus PE = 2 | | | | | | | | |
| | SV = 1 - [I3S764_LOTJA] | | | | | | | | |
| I3S8M2 | Uncharacterized protein | 7.24 | 7 | 1 | 1 | 1 | 152 | 16.2 | 5.27 |
| | OS = Lotus japonicus PE = 2 | | | | | | | | |
| | SV = 1 - [I3S8M2_LOTJA] | | | | | | | | |
| I3S9X5 | Uncharacterized protein | 8.77 | 2 | 1 | 1 | 1 | 114 | 13.5 | 7.56 |
| | OS = Lotus japonicus PE = 2 | | | | | | | | |
| | SV = 1 - [I3S9X5_LOTJA] | | | | | | | | |
| I3SBV7 | Uncharacterized protein | 19.33 | 5 | 1 | 2 | 2 | 119 | 13.6 | 9.45 |
| | OS = Lotus japonicus PE = 2 | | | | | | | | |
| | SV = 1 - [I3SBV7_LOTJA] | | | | | | | | |
| I3SC59 | Uncharacterized protein | 38.27 | 22 | 3 | 3 | 5 | 81 | 9.4 | 9.58 |
| | OS = Lotus japonicus PE = 2 | | | | | | | | |
| | SV = 1 - [I3SC59_LOTJA] | | | | | | | | |
| I3SD49 | Uncharacterized protein | 8.09 | 1 | 1 | 2 | 3 | 235 | 26.6 | 8.27 |
| | OS = Lotus japonicus PE = 2 | | | | | | | | |
| | SV = 1 - [I3SD49_LOTJA] | | | | | | | | |
| I3SE57 | Uncharacterized protein | 8.57 | 10 | 2 | 2 | 2 | 245 | 26.9 | 7.34 |
| | OS = Lotus japonicus PE = 2 | | | | | | | | |
| | SV = 1 - [I3SE57_LOTJA] | | | | | | | | |
| I3SGZ7 | Uncharacterized protein | 17.86 | 23 | 2 | 2 | 2 | 140 | 16.3 | 10.14 |
| | OS = Lotus japonicus PE = 2 | | | | | | | | |
| | SV = 1 - [I3SGZ7_LOTJA] | | | | | | | | |
| I3SH79 | Uncharacterized protein | 4.35 | 6 | 1 | 1 | 2 | 276 | 29.4 | 8.90 |
| | OS = Lotus japonicus PE = 2 | | | | | | | | |
| | SV = 1 - [I3SH79_LOTJA] | | | | | | | | |
| I3SHC8 | Uncharacterized protein | 24.32 | 4 | 1 | 6 | 6 | 259 | 29.4 | 10.18 |
| | OS = Lotus japonicus PE = 2 | | | | | | | | |
| | SV = 1 - [I3SHC8_LOTJA] | | | | | | | | |
| I3SI08 | Uncharacterized protein | 18.98 | 23 | 2 | 2 | 2 | 137 | 15.8 | 6.19 |
| | OS = Lotus japonicus PE = 2 | | | | | | | | |
| | SV = 1 - [I3SI08_LOTJA] | | | | | | | | |
| I3SKZ0 | Uncharacterized protein | 7.72 | 15 | 2 | 2 | 2 | 246 | 26.9 | 9.13 |
| | OS = Lotus japonicus PE = 2 | | | | | | | | |
| | SV = 1 - [I3SKZ0_LOTJA] | | | | | | | | |
| I3SLF3 | Uncharacterized protein | 6.86 | 10 | 3 | 3 | 3 | 423 | 43.0 | 6.40 |
| | OS = Lotus japonicus PE = 2 | | | | | | | | |
| | SV = 1 - [I3SLF3_LOTJA] | | | | | | | | |
| I3SLV8 | Uncharacterized protein | 8.44 | 16 | 3 | 3 | 3 | 320 | 35.0 | 8.91 |
| | OS = Lotus japonicus PE = 2 | | | | | | | | |
| | SV = 1 - [I3SLV8_LOTJA] | | | | | | | | |
| I3SN90 | Uncharacterized protein | 5.62 | 13 | 2 | 2 | 2 | 356 | 38.2 | 6.58 |
| | OS = Lotus japonicus PE = 2 | | | | | | | | |
| | SV = 1 - [I3SN90_LOTJA] | | | | | | | | |
| I3SR46 | Uncharacterized protein | 3.87 | 2 | 1 | 1 | 2 | 310 | 34.0 | 6.37 |
| | OS = Lotus japonicus PE = 2 | | | | | | | | |
| | SV = 1 - [I3SR46_LOTJA] | | | | | | | | |
| I3SR66 | Uncharacterized protein | 37.00 | 8 | 1 | 3 | 4 | 100 | 10.8 | 9.64 |
| | OS = Lotus japonicus PE = 2 | | | | | | | | |
| | SV = 1 - [I3SR66_LOTJA] | | | | | | | | |
| I3SS06 | Uncharacterized protein | 1.82 | 1 | 1 | 1 | 1 | 440 | 47.9 | 5.99 |
| | OS = Lotus japonicus PE = 2 | | | | | | | | |
| | SV = 1 - [I3SS06_LOTJA] | | | | | | | | |
| I3SS93 | Uncharacterized protein | 15.02 | 18 | 1 | 3 | 3 | 293 | 31.3 | 5.85 |
| | OS = Lotus japonicus PE = 2 | | | | | | | | |
| | SV = 1 - [I3SS93_LOTJA] | | | | | | | | |
| I3STL5 | Uncharacterized protein | 3.95 | 19 | 1 | 1 | 1 | 228 | 24.7 | 4.59 |
| | OS = Lotus japonicus PE = 2 | | | | | | | | |
| | SV = 1 - [I3STL5_LOTJA] | | | | | | | | |
| I3SU61 | Uncharacterized protein | 3.38 | 2 | 1 | 1 | 1 | 325 | 34.8 | 5.74 |
| | OS = Lotus japonicus PE = 2 | | | | | | | | |
| | SV = 1 - [I3SU61_LOTJA] | | | | | | | | |
| I3SU82 | Uncharacterized protein | 6.47 | 7 | 1 | 1 | 1 | 201 | 23.2 | 8.85 |
| | OS = Lotus japonicus PE = 2 | | | | | | | | |
| | SV = 1 - [I3SU82_LOTJA] | | | | | | | | |
| I3SW31 | Uncharacterized protein | 4.52 | 1 | 1 | 1 | 1 | 199 | 21.1 | 6.95 |
| | OS = Lotus japonicus PE = 2 | | | | | | | | |
| | SV = 1 - [I3SW31_LOTJA] | | | | | | | | |
| I3SYP3 | Uncharacterized protein | 14.68 | 6 | 2 | 3 | 14 | 293 | 31.8 | 7.72 |
| | OS = Lotus japonicus PE = 2 | | | | | | | | |
| | SV = 1 - [I3SYP3_LOTJA] | | | | | | | | |
| I3T2V1 | Uncharacterized protein | 8.75 | 11 | 2 | 2 | 2 | 320 | 36.4 | 6.79 |
| | OS = Lotus japonicus PE = 2 | | | | | | | | |
| | SV = 1 - [I3T2V1_LOTJA] | | | | | | | | |
| I3T5D3 | Uncharacterized protein | 40.00 | 9 | 1 | 1 | 1 | 25 | 2.8 | 10.45 |
| | OS = Lotus japonicus PE = 2 | | | | | | | | |
| | SV = 1 - [I3T5D3_LOTJA] | | | | | | | | |
| I3T649 | Uncharacterized protein | 13.33 | 1 | 1 | 1 | 1 | 90 | 10.1 | 6.74 |
| | OS = Lotus japonicus PE = 2 | | | | | | | | |
| | SV = 1 - [I3T649_LOTJA] | | | | | | | | |
| I3T6L1 | Uncharacterized protein | 15.64 | 21 | 2 | 2 | 2 | 179 | 20.0 | 4.79 |
| | OS = Lotus japonicus PE = 2 | | | | | | | | |
| | SV = 1 - [I3T6L1_LOTJA] | | | | | | | | |
| I3T6L5 | Uncharacterized protein | 4.41 | 15 | 2 | 2 | 2 | 476 | 52.1 | 8.92 |
| | OS = Lotus japonicus PE = 2 | | | | | | | | |
| | SV = 1 - [I3T6L5_LOTJA] | | | | | | | | |
| I3T6Q1 | Uncharacterized protein | 8.28 | 3 | 1 | 1 | 1 | 157 | 16.6 | 8.25 |
| | OS = Lotus japonicus PE = 2 | | | | | | | | |
| | SV = 1 - [I3T6Q1_LOTJA] | | | | | | | | |
| G7IEE7 | Uncharacterized protein | 5.06 | 3 | 2 | 2 | 2 | 435 | 46.5 | 8.19 |
| | OS = Medicago truncatula | | | | | | | | |
| | GN = MTR_1g072420 | | | | | | | | |
| | PE = 4 SV = 1 - | | | | | | | | |
| | [G7IEE7_MEDTR] | | | | | | | | |
| A0A072VQF0 | Uncharacterized protein | 4.35 | 12 | 2 | 2 | 2 | 529 | 57.9 | 5.67 |
| | OS = Medicago truncatula | | | | | | | | |
| | GN = MTR_1g106940 | | | | | | | | |
| | PE = 4 SV = 1 - | | | | | | | | |
| | [A0A072VQF0_MEDTR] | | | | | | | | |
| G7IGW6 | Uncharacterized protein | 2.62 | 2 | 1 | 1 | 1 | 267 | 31.2 | 9.35 |
| | OS = Medicago truncatula | | | | | | | | |
| | GN = MTR_2g064120 | | | | | | | | |
| | PE = 4 SV = 1 - | | | | | | | | |
| | [G7IGW6_MEDTR] | | | | | | | | |
| A0A072V0U2 | Uncharacterized protein | 20.26 | 61 | 2 | 7 | 13 | 380 | 41.1 | 6.40 |
| | OS = Medicago truncatula | | | | | | | | |
| | GN = MTR_3g089977 | | | | | | | | |
| | PE = 3 SV = 1 - | | | | | | | | |
| | [A0A072V0U2_MEDTR] | | | | | | | | |
| A0A072UTE0 | Uncharacterized protein | 17.74 | 1 | 1 | 1 | 1 | 62 | 7.1 | 9.82 |
| | OS = Medicago truncatula | | | | | | | | |
| | GN = MTR_4g026995 | | | | | | | | |
| | PE = 4 SV = 1 - | | | | | | | | |
| | [A0A072UTE0_MEDTR] | | | | | | | | |
| G7K3T7 | Uncharacterized protein | 5.47 | 1 | 1 | 1 | 1 | 128 | 14.5 | 5.48 |
| | OS = Medicago truncatula | | | | | | | | |
| | GN = MTR_5g015700 | | | | | | | | |
| | PE = 4 SV = 1 - | | | | | | | | |
| | [G7K3T7_MEDTR] | | | | | | | | |
| B7FGK9 | Uncharacterized protein | 6.08 | 18 | 1 | 1 | 1 | 148 | 16.9 | 10.76 |
| | OS = Medicago truncatula | | | | | | | | |
| | PE = 2 SV = 1 - | | | | | | | | |
| | [B7FGK9_MEDTR] | | | | | | | | |
| B7FIV8 | Uncharacterized protein | 9.91 | 14 | 1 | 2 | 2 | 323 | 35.9 | 5.72 |
| | OS = Medicago truncatula | | | | | | | | |
| | PE = 2 SV = 1 - | | | | | | | | |
| | [B7FIV8_MEDTR] | | | | | | | | |
| B7FJC8 | Uncharacterized protein | 31.56 | 46 | 2 | 9 | 11 | 377 | 41.6 | 5.49 |
| | OS = Medicago truncatula | | | | | | | | |
| | PE = 2 SV = 1 - | | | | | | | | |
| | [B7FJC8_MEDTR] | | | | | | | | |
| I3S421 | Uncharacterized protein | 4.96 | 3 | 1 | 1 | 1 | 141 | 16.5 | 8.76 |
| | OS = Medicago truncatula | | | | | | | | |
| | PE = 2 SV = 1 - | | | | | | | | |
| | [I3S421_MEDTR] | | | | | | | | |
| I3S4X7 | Uncharacterized protein | 11.11 | 14 | 1 | 1 | 2 | 81 | 9.1 | 12.31 |
| | OS = Medicago truncatula | | | | | | | | |
| | PE = 2 SV = 1 - | | | | | | | | |
| | [I3S4X7_MEDTR] | | | | | | | | |
| I3S5U3 | Uncharacterized protein | 11.11 | 5 | 1 | 1 | 1 | 162 | 17.2 | 6.52 |
| | OS = Medicago truncatula | | | | | | | | |
| | PE = 2 SV = 1 - | | | | | | | | |
| | [I3S5U3_MEDTR] | | | | | | | | |
| I3S7L1 | Uncharacterized protein | 6.67 | 23 | 1 | 1 | 1 | 150 | 16.6 | 8.76 |
| | OS = Medicago truncatula | | | | | | | | |
| | PE = 2 SV = 1 - | | | | | | | | |
| | [I3S7L1_MEDTR] | | | | | | | | |
| I3S7Z6 | Uncharacterized protein | 6.82 | 10 | 1 | 1 | 1 | 176 | 18.5 | 4.56 |
| | OS = Medicago truncatula | | | | | | | | |
| | PE = 2 SV = 1 - | | | | | | | | |
| | [I3S7Z6_MEDTR] | | | | | | | | |
| I3S9B0 | Uncharacterized protein | 12.21 | 10 | 1 | 2 | 2 | 172 | 19.2 | 7.88 |
| | OS = Medicago truncatula | | | | | | | | |
| | PE = 2 SV = 1 - | | | | | | | | |
| | [I3S9B0_MEDTR] | | | | | | | | |
| I3SBU9 | Uncharacterized protein | 15.46 | 22 | 1 | 2 | 2 | 194 | 20.9 | 7.39 |
| | OS = Medicago truncatula | | | | | | | | |
| | PE = 2 SV = 1 - | | | | | | | | |
| | [I3SBU9_MEDTR] | | | | | | | | |
| I3SG13 | Uncharacterized protein | 2.32 | 4 | 1 | 1 | 1 | 345 | 38.4 | 4.82 |
| | OS = Medicago truncatula | | | | | | | | |
| | PE = 2 SV = 1 - | | | | | | | | |
| | [I3SG13_MEDTR] | | | | | | | | |
| I3SGG7 | Uncharacterized protein | 18.23 | 34 | 3 | 3 | 3 | 181 | 20.7 | 6.55 |
| | OS = Medicago truncatula | | | | | | | | |
| | PE = 2 SV = 1 - | | | | | | | | |
| | [I3SGG7_MEDTR] | | | | | | | | |
| I3SHW5 | Uncharacterized protein | 6.38 | 14 | 1 | 1 | 1 | 141 | 15.2 | 8.46 |
| | OS = Medicago truncatula | | | | | | | | |
| | PE = 2 SV = 1 - | | | | | | | | |
| | [I3SHW5_MEDTR] | | | | | | | | |
| I3SHZ6 | Uncharacterized protein | 9.43 | 23 | 1 | 1 | 1 | 106 | 12.0 | 5.57 |
| | OS = Medicago truncatula | | | | | | | | |
| | PE = 2 SV = 1 - | | | | | | | | |
| | [I3SHZ6_MEDTR] | | | | | | | | |
| I3SVI6 | Uncharacterized protein | 2.12 | 8 | 1 | 1 | 1 | 378 | 41.2 | 7.94 |
| | OS = Medicago truncatula | | | | | | | | |
| | PE = 2 SV = 1 - | | | | | | | | |
| | [I3SVI6_MEDTR] | | | | | | | | |
| I3SW63 | Uncharacterized protein | 3.51 | 9 | 1 | 1 | 1 | 285 | 31.1 | 10.90 |
| | OS = Medicago truncatula | | | | | | | | |
| | PE = 2 SV = 1 - | | | | | | | | |
| | [I3SW63_MEDTR] | | | | | | | | |
| I3T3E0 | Uncharacterized protein | 18.39 | 9 | 1 | 1 | 1 | 87 | 9.4 | 6.04 |
| | OS = Medicago truncatula | | | | | | | | |
| | PE = 2 SV = 1 - | | | | | | | | |
| | [I3T3E0_MEDTR] | | | | | | | | |
| I3T4U7 | Uncharacterized protein | 29.70 | 35 | 5 | 5 | 6 | 202 | 23.1 | 8.73 |
| | OS = Medicago truncatula | | | | | | | | |
| | PE = 2 SV = 1 - | | | | | | | | |
| | [I3T4U7_MEDTR] | | | | | | | | |
| A0A0L9TJ62 | Uncharacterized protein | 1.06 | 5 | 1 | 1 | 1 | 947 | 103.7 | 6.33 |
| | OS = Phaseolus angularis | | | | | | | | |
| | GN = LR48_Vigan01g005500 | | | | | | | | |
| | PE = 4 SV = 1 - | | | | | | | | |
| | [A0A0L9TJ62_PHAAN] | | | | | | | | |
| A0A0L9TJH4 | Uncharacterized protein | 6.98 | 4 | 1 | 2 | 2 | 315 | 35.9 | 7.06 |
| | OS = Phaseolus angularis | | | | | | | | |
| | GN = LR48_Vigan01g026400 | | | | | | | | |
| | PE = 4 SV = 1 - | | | | | | | | |
| | [A0A0L9TJH4_PHAAN] | | | | | | | | |
| A0A0L9TK03 | Uncharacterized protein | 1.61 | 2 | 1 | 1 | 1 | 560 | 62.3 | 6.64 |
| | OS = Phaseolus angularis | | | | | | | | |
| | GN = LR48_Vigan01g032700 | | | | | | | | |
| | PE = 4 SV = 1 - | | | | | | | | |
| | [A0A0L9TK03_PHAAN] | | | | | | | | |
| A0A0L9TMD8 | Uncharacterized protein | 2.78 | 5 | 1 | 1 | 1 | 396 | 45.6 | 4.86 |
| | OS = Phaseolus angularis | | | | | | | | |
| | GN = LR48_Vigan01g131300 | | | | | | | | |
| | PE = 4 SV = 1 - | | | | | | | | |
| | [A0A0L9TMD8_PHAAN] | | | | | | | | |
| A0A0L9TMU2 | Uncharacterized protein | 10.00 | 1 | 1 | 1 | 2 | 130 | 14.1 | 5.26 |
| | OS = Phaseolus angularis | | | | | | | | |
| | GN = LR48_Vigan01g143600 | | | | | | | | |
| | PE = 4 SV = 1 - | | | | | | | | |
| | [A0A0L9TMU2_PHAAN] | | | | | | | | |
| A0A0L9TPU8 | Uncharacterized protein | 26.91 | 10 | 5 | 5 | 6 | 249 | 27.1 | 7.44 |
| | OS = Phaseolus angularis | | | | | | | | |
| | GN = LR48_Vigan01g1175500 | | | | | | | | |
| | PE = 4 SV = 1 - | | | | | | | | |
| | [A0A0L9TPU8_PHAAN] | | | | | | | | |
| A0A0L9TQF5 | Uncharacterized protein | 8.62 | 12 | 4 | 4 | 4 | 557 | 63.6 | 7.43 |
| | OS = Phaseolus angularis | | | | | | | | |
| | GN = LR48_Vigan01g226700 | | | | | | | | |
| | PE = 4 SV = 1 - | | | | | | | | |
| | [A0A0L9TQF5_PHAAN] | | | | | | | | |
| A0A0L9TQU6 | Uncharacterized protein | 20.65 | 44 | 0 | 10 | 16 | 649 | 71.0 | 5.39 |
| | OS = Phaseolus angularis | | | | | | | | |
| | GN = LR48_Vigan01g242700 | | | | | | | | |
| | PE = 4 SV = 1 - | | | | | | | | |
| | [A0A0L9TQU6_PHAAN] | | | | | | | | |
| A0A0L9TR38 | Uncharacterized protein | 18.75 | 2 | 1 | 1 | 1 | 128 | 13.9 | 7.12 |
| | OS = Phaseolus angularis | | | | | | | | |
| | GN = LR48_Vigan01g259900 | | | | | | | | |
| | PE = 4 SV = 1 - | | | | | | | | |
| | [A0AL9TR38_PHAAN] | | | | | | | | |
| A0A0L9TSP3 | Uncharacterized protein | 2.77 | 17 | 1 | 1 | 1 | 470 | 51.8 | 5.71 |
| | OS = Phaseolus angularis | | | | | | | | |
| | GN = LR48_Vigan01g274500 | | | | | | | | |
| | PE = 4 SV = 1 - | | | | | | | | |
| | [A0A0L9TSP3_PHAAN] | | | | | | | | |
| A0A0L9TRN7 | Uncharacterized protein | 25.62 | 42 | 3 | 3 | 3 | 160 | 17.5 | 5.99 |
| | OS = Phaseolus angularis | | | | | | | | |
| | GN = LR48_Vigan01g276100 | | | | | | | | |
| | PE = 4 SV = 1 - | | | | | | | | |
| | [A0A0L9TRN7_PHAAN] | | | | | | | | |
| A0A0L9TS29 | Uncharacterized protein | 10.00 | 12 | 4 | 4 | 4 | 500 | 52.8 | 7.34 |
| | OS = Phaseolus angularis | | | | | | | | |
| | GN = LR48_Vigan01g294900 | | | | | | | | |
| | PE = 4 SV = 1 - | | | | | | | | |
| | [A0A0L9TS29_PHAAN] | | | | | | | | |
| A0A0L9TTZ2 | Uncharacterized protein | 18.46 | 6 | 1 | 1 | 1 | 65 | 7.5 | 11.17 |
| | OS = Phaseolus angularis | | | | | | | | |
| | GN = LR48_Vigan02g020800 | | | | | | | | |
| | PE = 4 SV = 1 - | | | | | | | | |
| | [A0A0L9TTZ2_PHAAN] | | | | | | | | |
| A0A0L9TUV3 | Uncharacterized protein | 7.76 | 3 | 1 | 2 | 2 | 438 | 47.9 | 6.80 |
| | OS = Phaseolus angularis | | | | | | | | |
| | GN = LR48_Vigan02g043100 | | | | | | | | |
| | PE = 4 SV = 1 - | | | | | | | | |
| | [A0A0L9TUV3_PHAAN] | | | | | | | | |
| A0A0L9TW71 | Uncharacterized protein | 12.32 | 12 | 1 | 3 | 4 | 357 | 38.4 | 7.34 |
| | OS = Phaseolus angularis | | | | | | | | |
| | GN = LR48_Vigan02g080200 | | | | | | | | |
| | PE = 4 SV = 1 - | | | | | | | | |
| | [A0A0L9TW71_PHAAN] | | | | | | | | |
| A0A0L9TWZ0 | Uncharacterized protein | 19.21 | 18 | 2 | 2 | 3 | 151 | 17.1 | 10.40 |
| | OS = Phaseolus angularis | | | | | | | | |
| | GN = LR48_Vigan02g125300 | | | | | | | | |
| | PE = 4 SV = 1 - | | | | | | | | |
| | [A0A0L9TWZ0_PHAAN] | | | | | | | | |
| A0A0L9TXW4 | Uncharacterized protein | 1.45 | 1 | 1 | 1 | 1 | 688 | 75.1 | 6.54 |
| | OS = Phaseolus angularis | | | | | | | | |
| | GN = LR48_Vigan02g139200 | | | | | | | | |
| | PE = 4 SV = 1 - | | | | | | | | |
| | [A0A0L9TXW4_PHAAN] | | | | | | | | |
| A0A0L9TYP0 | Uncharacterized protein | 12.40 | 17 | 4 | 4 | 5 | 379 | 40.7 | 7.02 |
| | OS = Phaseolus angularis | | | | | | | | |
| | GN = LR48_Vigan02g144900 | | | | | | | | |
| | PE = 4 SV = 1 - | | | | | | | | |
| | [A0A0L9TYP0_PHAAN] | | | | | | | | |
| A0A0L9TY25 | Uncharacterized protein | 8.81 | 6 | 1 | 4 | 4 | 420 | 48.2 | 4.65 |
| | OS = Phaseolus angularis | | | | | | | | |
| | GN = LR48_Vigan02g161800 | | | | | | | | |
| | PE = 4 SV = 1 - | | | | | | | | |
| | [A0A0L9TY25_PHAAN] | | | | | | | | |
| A0A0L9TZG1 | Uncharacterized protein | 7.76 | 3 | 2 | 2 | 2 | 245 | 27.6 | 6.32 |
| | OS = Phaseolus angularis | | | | | | | | |
| | GN = LR48_Vigan02g195700 | | | | | | | | |
| | PE = 4 SV = 1 - | | | | | | | | |
| | [A0A0L9TZG1_PHAAN] | | | | | | | | |
| A0A0L9TZ34 | Uncharacterized protein | 26.32 | 11 | 2 | 3 | 3 | 152 | 17.5 | 10.56 |
| | OS = Phaseolus angularis | | | | | | | | |
| | GN = LR48_Vigan02g199800 | | | | | | | | |
| | PE = 4 SV = 1 - | | | | | | | | |
| | [A0A0L9TZ34_PHAAN] | | | | | | | | |
| A0A0L9TZG5 | Uncharacterized protein | 2.87 | 5 | 1 | 4 | 4 | 1357 | 150.3 | 6.73 |
| | OS = Phaseolus angularis | | | | | | | | |
| | GN = LR48_Vigan02g210800 | | | | | | | | |
| | PE = 4 SV = 1 - | | | | | | | | |
| | [A0A0L9TZG5_PHAAN] | | | | | | | | |
| A0A0L9U0X7 | Uncharacterized protein | 21.46 | 10 | 2 | 5 | 5 | 261 | 29.7 | 9.82 |
| | OS = Phaseolus angularis | | | | | | | | |
| | GN = LR48_Vigan02g221900 | | | | | | | | |
| | PE = 4 SV = 1 - | | | | | | | | |
| | [A0A0L9U0X7_PHAAN] | | | | | | | | |
| A0A0L9U1P6 | Uncharacterized protein | 4.88 | 12 | 1 | 1 | 1 | 328 | 36.6 | 5.54 |
| | OS = Phaseolus angularis | | | | | | | | |
| | GN = LR48_Vigan02g248900 | | | | | | | | |
| | PE = 4 SV = 1 - | | | | | | | | |
| | [A0A0L9U1P6_PHAAN] | | | | | | | | |
| A0A0L9U390 | Uncharacterized protein | 16.67 | 21 | 2 | 2 | 2 | 120 | 13.7 | 11.60 |
| | OS = Phaseolus angularis | | | | | | | | |
| | GN = LR48_Vigan03g025000 | | | | | | | | |
| | PE = 4 SV = 1 - | | | | | | | | |
| | [A0A0L9U390_PHAAN] | | | | | | | | |
| A0A0L9U2M4 | Uncharacterized protein | 5.42 | 10 | 1 | 1 | 1 | 166 | 19.1 | 5.43 |
| | OS = Phaseolus angularis | | | | | | | | |
| | GN = LR48_Vigan03g039700 | | | | | | | | |
| | PE = 4 SV = 1 - | | | | | | | | |
| | [A0A0L9U2M4_PHAAN] | | | | | | | | |
| A0A0L9U447 | Uncharacterized protein | 6.90 | 11 | 1 | 1 | 1 | 116 | 13.1 | 4.48 |
| | OS = Phaseolus angularis | | | | | | | | |
| | GN = LR48_Vigan03g056500 | | | | | | | | |
| | PE = 4 SV = 1 - | | | | | | | | |
| | [A0A0L9U447_PHAAN] | | | | | | | | |
| A0A0L9U3P8 | Uncharacterized protein | 3.94 | 5 | 1 | 2 | 2 | 559 | 60.7 | 5.78 |
| | OS = Phaseolus angularis | | | | | | | | |
| | GN = LR48_Vigan03g068800 | | | | | | | | |
| | PE = 4 SV = 1 - | | | | | | | | |
| | [A0A0L9U3P8_PHAAN] | | | | | | | | |
| A0A0L9U3H1 | Uncharacterized protein | 2.58 | 2 | 1 | 1 | 2 | 619 | 68.5 | 5.38 |
| | OS = Phaseolus angularis | | | | | | | | |
| | GN = LR48_Vigan03g075000 | | | | | | | | |
| | PE = 4 SV = 1 - | | | | | | | | |
| | [A0A0L9U3H1_PHAAN] | | | | | | | | |
| A0A0L9U547 | Uncharacterized protein | 32.72 | 29 | 6 | 6 | 9 | 217 | 24.6 | 10.36 |
| | OS = Phaseolus angularis | | | | | | | | |
| | GN = LR48_Vigan03g118400 | | | | | | | | |
| | PE = 4 SV = 1 - | | | | | | | | |
| | [A0A0L9U547_PHAAN] | | | | | | | | |
| A0A0L9U7Z5 | Uncharacterized protein | 15.86 | 17 | 0 | 10 | 16 | 763 | 84.2 | 6.35 |
| | OS = Phaseolus angularis | | | | | | | | |
| | GN = LR48_Vigan03g195100 | | | | | | | | |
| | PE = 4 SV = 1 - | | | | | | | | |
| | [A0A0L9U7Z5_PHAAN] | | | | | | | | |
| A0A0L9U8A1 | Uncharacterized protein | 1.93 | 6 | 1 | 1 | 1 | 673 | 73.8 | 5.29 |
| | OS = Phaseolus angularis | | | | | | | | |
| | GN = LR48_Vigan03g206600 | | | | | | | | |
| | PE = 4 SV = 1 - | | | | | | | | |
| | [A0A0L9U8A1_PHAAN] | | | | | | | | |
| A0A0L9U7B0 | Uncharacterized protein | 20.45 | 16 | 1 | 2 | 3 | 132 | 14.2 | 5.31 |
| | OS = Phaseolus angularis | | | | | | | | |
| | GN = LR48_Vigan03g207500 | | | | | | | | |
| | PE = 4 SV = 1 - | | | | | | | | |
| | [A0A0L9U7B0_PHAAN] | | | | | | | | |
| A0A0L9U8T5 | Uncharacterized protein | 5.11 | 10 | 1 | 2 | 2 | 450 | 49.1 | 6.98 |
| | OS = Phaseolus angularis | | | | | | | | |
| | GN = LR48_Vigan03g228600 | | | | | | | | |
| | PE = 4 SV = 1 - | | | | | | | | |
| | [A0A0L9U8T5_PHAAN] | | | | | | | | |
| A0A0L9U987 | Uncharacterized protein | 13.21 | 11 | 1 | 1 | 1 | 53 | 5.8 | 4.55 |
| | OS = Phaseolus angularis | | | | | | | | |
| | GN = LR48_Vigan03g244600 | | | | | | | | |
| | PE = 4 SV = 1 - | | | | | | | | |
| | [A0A0L9U987_PHAAN] | | | | | | | | |
| A0A0L9UA16 | Uncharacterized protein | 7.63 | 11 | 1 | 1 | 1 | 118 | 12.9 | 11.12 |
| | OS = Phaseolus angularis | | | | | | | | |
| | GN = LR48_Vigan03g275600 | | | | | | | | |
| | PE = 4 SV = 1 - | | | | | | | | |
| | [A0A0L9UA16_PHAAN] | | | | | | | | |
| A0A0L9UB16 | Uncharacterized protein | 21.47 | 32 | 1 | 6 | 27 | 340 | 36.9 | 7.05 |
| | OS = Phaseolus angularis | | | | | | | | |
| | GN = LR48_Vigan03g313400 | | | | | | | | |
| | PE = 4 SV = 1 - | | | | | | | | |
| | [A0A0L9UB16_PHAAN] | | | | | | | | |
| A0A0L9UFR2 | Uncharacterized protein | 10.56 | 8 | 1 | 1 | 1 | 180 | 20.0 | 5.95 |
| | OS = Phaseolus angularis | | | | | | | | |
| | GN = LR48_Vigan04g176100 | | | | | | | | |
| | PE = 4 SV = 1 - | | | | | | | | |
| | [A0A0L9UFR2_PHAAN] | | | | | | | | |
| A0A0L9UFV4 | Uncharacterized protein | 4.01 | 14 | 1 | 1 | 1 | 424 | 46.9 | 7.21 |
| | OS = Phaseolus angularis | | | | | | | | |
| | GN = LR48_Vigan04g199300 | | | | | | | | |
| | PE = 4 SV = 1 - | | | | | | | | |
| | [A0A0L9UFV4_PHAAN] | | | | | | | | |
| A0A0L9UHV4 | Uncharacterized protein | 5.15 | 7 | 2 | 4 | 6 | 873 | 98.3 | 5.34 |
| | OS = Phaseolus angularis | | | | | | | | |
| | GN = LR48_Vigan04g234000 | | | | | | | | |
| | PE = 4 SV = 1 - | | | | | | | | |
| | [A0A0L9UHV4_PHAAN] | | | | | | | | |
| A0A0L9UJ87 | Uncharacterized protein | 14.23 | 8 | 2 | 11 | 11 | 822 | 91.4 | 5.88 |
| | OS = Phaseolus angularis | | | | | | | | |
| | GN = LR48_Vigan05g022600 | | | | | | | | |
| | PE = 4 SV = 1 - | | | | | | | | |
| | [A0A0L9UJ87_PHAAN] | | | | | | | | |
| A0A0L9UJH6 | Uncharacterized protein | 5.67 | 5 | 1 | 2 | 2 | 476 | 49.8 | 8.73 |
| | OS = Phaseolus angularis | | | | | | | | |
| | GN = LR48_Vigan05g031600 | | | | | | | | |
| | PE = 4 SV = 1 - | | | | | | | | |
| | [A0A0L9UJH6_PHAAN] | | | | | | | | |
| A0A0L9UK60 | Uncharacterized protein | 9.57 | 14 | 1 | 1 | 1 | 115 | 12.9 | 10.21 |
| | OS = Phaseolus angularis | | | | | | | | |
| | GN = LR48_Vigan05g088900 | | | | | | | | |
| | PE = 4 SV = 1 - | | | | | | | | |
| | [A0A0L9UK60_PHAAN] | | | | | | | | |
| A0A0L9UM32 | Uncharacterized protein | 3.14 | 1 | 1 | 1 | 2 | 509 | 53.8 | 8.76 |
| | OS = Phaseolus angularis | | | | | | | | |
| | GN = LR48_Vigan05g137600 | | | | | | | | |
| | PE = 4 SV = 1 - | | | | | | | | |
| | [A0A0L9UM32_PHAAN] | | | | | | | | |
| A0A0L9UPA1 | Uncharacterized protein | 11.23 | 54 | 2 | 8 | 14 | 855 | 96.1 | 6.80 |
| | OS = Phaseolus angularis | | | | | | | | |
| | GN = LR48_Vigan05g201700 | | | | | | | | |
| | PE = 4 SV = 1 - | | | | | | | | |
| | [A0A0L9UPA1_PHAAN] | | | | | | | | |
| A0A0L9UPP1 | Uncharacterized protein | 3.40 | 11 | 2 | 2 | 2 | 852 | 94.1 | 5.26 |
| | OS = Phaseolus angularis | | | | | | | | |
| | GN = LR48_Vigan05g214200 | | | | | | | | |
| | PE = 4 SV = 1 - | | | | | | | | |
| | [A0A0L9UPP1_PHAAN] | | | | | | | | |
| A0A0L9UPL8 | Uncharacterized protein | 37.86 | 23 | 4 | 4 | 5 | 140 | 14.9 | 5.77 |
| | OS = Phaseolus angularis | | | | | | | | |
| | GN = LR48_Vigan05g226600 | | | | | | | | |
| | PE = 4 SV = 1 - | | | | | | | | |
| | [A0A0L9UPL8_PHAAN] | | | | | | | | |
| A0A0L9UPR9 | Uncharacterized protein | 24.69 | 7 | 2 | 2 | 2 | 162 | 17.7 | 5.63 |
| | OS = Phaseolus angularis | | | | | | | | |
| | GN = LR48_Vigan06g001700 | | | | | | | | |
| | PE = 4 SV = 1 - | | | | | | | | |
| | [A0A0L9UPR9_PHAAN] | | | | | | | | |
| A0A0L9UPW3 | Uncharacterized protein | 3.61 | 10 | 1 | 2 | 2 | 582 | 65.5 | 6.77 |
| | OS = Phaseolus angularis | | | | | | | | |
| | GN = LR48_Vigan06g008900 | | | | | | | | |
| | PE = 4 SV = 1 - | | | | | | | | |
| | [A0A0L9UPW3_PHAAN] | | | | | | | | |
| A0A0L9URB4 | Uncharacterized protein | 5.16 | 8 | 1 | 1 | 1 | 543 | 64.6 | 5.26 |
| | OS = Phaseolus angularis | | | | | | | | |
| | GN = LR48_Vigan06g042800 | | | | | | | | |
| | PE = 4 SV = 1 - | | | | | | | | |
| | [A0A0L9URB4_PHAAN] | | | | | | | | |
| A0A0L9USF0 | Uncharacterized protein | 11.68 | 7 | 1 | 1 | 1 | 137 | 15.2 | 8.12 |
| | OS = Phaseolus angularis | | | | | | | | |
| | GN = LR48_Vigan06g098900 | | | | | | | | |
| | PE = 4 SV = 1 - | | | | | | | | |
| | [A0A0L9USF0_PHAAN] | | | | | | | | |
| A0A0L9UT10 | Uncharacterized protein | 5.84 | 2 | 1 | 1 | 1 | 137 | 15.2 | 8.69 |
| | OS = Phaseolus angularis | | | | | | | | |
| | GN = LR48_Vigan06g099000 | | | | | | | | |
| | PE = 4 SV = 1 - | | | | | | | | |
| | [A0A0L9UT10_PHAAN] | | | | | | | | |
| A0A0L9UT98 | Uncharacterized protein | 2.88 | 8 | 2 | 2 | 2 | 730 | 78.9 | 6.90 |
| | OS = Phaseolus angularis | | | | | | | | |
| | GN = LR48_Vigan06g111800 | | | | | | | | |
| | PE = 4 SV = 1 - | | | | | | | | |
| | [A0A0L9UT98_PHAAN] | | | | | | | | |
| A0A0L9UUX9 | Uncharacterized protein | 3.82 | 13 | 1 | 1 | 1 | 288 | 32.2 | 5.41 |
| | OS = Phaseolus angularis | | | | | | | | |
| | GN = LR48_Vigan07g025700 | | | | | | | | |
| | PE = 4 SV = 1 - | | | | | | | | |
| | [A0A0L9UUX9_PHAAN] | | | | | | | | |
| A0A0L9VHC1 | Uncharacterized protein | 17.39 | 4 | 1 | 1 | 1 | 69 | 7.7 | 9.72 |
| | OS = Phaseolus angularis | | | | | | | | |
| | GN = LR48_Vigan07g054300 | | | | | | | | |
| | PE = 4 SV = 1 - | | | | | | | | |
| | [A0A0L9VHC1_PHAAN] | | | | | | | | |
| A0A0L9UVK9 | Uncharacterized protein | 9.57 | 19 | 1 | 1 | 2 | 94 | 11.0 | 10.08 |
| | OS = Phaseolus angularis | | | | | | | | |
| | GN = LR48_Vigan07g059900 | | | | | | | | |
| | PE = 4 SV = 1 - | | | | | | | | |
| | [A0A0L9UVK9_PHAAN] | | | | | | | | |
| A0A0L9UVW3 | Uncharacterized protein | 2.52 | 2 | 1 | 1 | 1 | 595 | 67.9 | 6.28 |
| | OS = Phaseolus angularis | | | | | | | | |
| | GN = LR48_Vigan07g060700 | | | | | | | | |
| | PE = 4 SV = 1 - | | | | | | | | |
| | [A0A0L9UVW3_PHAAN] | | | | | | | | |
| A0A0L9UWM1 | Uncharacterized protein | 21.79 | 7 | 1 | 3 | 6 | 156 | 17.6 | 7.42 |
| | OS = Phaseolus angularis | | | | | | | | |
| | GN = LR48_Vigan07g065600 | | | | | | | | |
| | PE = 4 SV = 1 - | | | | | | | | |
| | [A0A0L9UWM1_PHAAN] | | | | | | | | |
| A0A0L9UW85 | Uncharacterized protein | 3.37 | 1 | 1 | 1 | 2 | 563 | 60.4 | 6.61 |
| | OS = Phaseolus angularis | | | | | | | | |
| | GN = LR48_Vigan07g082400 | | | | | | | | |
| | PE = 4 SV = 1 - | | | | | | | | |
| | [A0A0L9UW85_PHAAN] | | | | | | | | |
| A0A0L9UZW8 | Uncharacterized protein | 21.51 | 17 | 1 | 5 | 7 | 358 | 38.4 | 7.01 |
| | OS = Phaseolus angularis | | | | | | | | |
| | GN = LR48_Vigan07g183600 | | | | | | | | |
| | PE = 4 SV = 1 - | | | | | | | | |
| | [A0A0L9UZW8_PHAAN] | | | | | | | | |
| A0A0L9V0P8 | Uncharacterized protein | 2.24 | 2 | 1 | 1 | 1 | 447 | 48.1 | 8.34 |
| | OS = Phaseolus angularis | | | | | | | | |
| | GN = LR48_Vigan07g203800 | | | | | | | | |
| | PE = 4 SV = 1 - | | | | | | | | |
| | [A0A0L9V0P8_PHAAN] | | | | | | | | |
| A0A0L9V1U0 | Uncharacterized protein | 11.52 | 5 | 1 | 8 | 8 | 816 | 93.4 | 4.97 |
| | OS = Phaseolus angularis | | | | | | | | |
| | GN = LR48_Vigan07g277900 | | | | | | | | |
| | PE = 4 SV = 1 - | | | | | | | | |
| | [A0A0L9V1U0_PHAAN] | | | | | | | | |
| A0A0L9V379 | Uncharacterized protein | 13.01 | 7 | 2 | 2 | 3 | 246 | 27.4 | 5.86 |
| | OS = Phaseolus angularis | | | | | | | | |
| | GN = LR48_Vigan08g032200 | | | | | | | | |
| | PE = 4 SV = 1 - | | | | | | | | |
| | [A0A0L9V379_PHAAN] | | | | | | | | |
| A0A0L9V4G6 | Uncharacterized protein | 9.88 | 8 | 2 | 2 | 3 | 243 | 26.3 | 6.32 |
| | OS = Phaseolus angularis | | | | | | | | |
| | GN = LR48_Vigan08g042600 | | | | | | | | |
| | PE = 4 SV = 1 - | | | | | | | | |
| | [A0A0L9V4G6_PHAAN] | | | | | | | | |
| A0A0L9V7U5 | Uncharacterized protein | 8.44 | 5 | 1 | 1 | 1 | 154 | 17.9 | 6.80 |
| | OS = Phaseolus angularis | | | | | | | | |
| | GN = LR48_Vigan08g158100 | | | | | | | | |
| | PE = 4 SV = 1 - | | | | | | | | |
| | [A0A0L9V7U5_PHAAN] | | | | | | | | |
| A0A0L9V7K6 | Uncharacterized protein | 2.91 | 5 | 1 | 1 | 1 | 309 | 34.7 | 6.99 |
| | OS = Phaseolus angularis | | | | | | | | |
| | GN = LR48_Vigan08g178000 | | | | | | | | |
| | PE = 4 SV = 1 - | | | | | | | | |
| | [A0A0L9V7K6_PHAAN] | | | | | | | | |
| A0A0L9V7T5 | Uncharacterized protein | 3.11 | 4 | 1 | 1 | 1 | 450 | 50.0 | 6.19 |
| | OS = Phaseolus angularis | | | | | | | | |
| | GN = LR48_Vigan08g187000 | | | | | | | | |
| | PE = 4 SV = 1 - | | | | | | | | |
| | [A0A0L9V7T5_PHAAN] | | | | | | | | |
| A0A0L9VA34 | Uncharacterized protein | 10.64 | 9 | 1 | 1 | 1 | 94 | 11.1 | 8.31 |
| | OS = Phaseolus angularis | | | | | | | | |
| | GN = LR48_Vigan09g015300 | | | | | | | | |
| | PE = 4 SV = 1 - | | | | | | | | |
| | [A0A0L9VA34_PHAAN] | | | | | | | | |
| A0A0L9VAB5 | Uncharacterized protein | 3.35 | 4 | 1 | 1 | 1 | 358 | 40.1 | 8.12 |
| | OS = Phaseolus angularis | | | | | | | | |
| | GN = LR48_Vigan09g025800 | | | | | | | | |
| | PE = 4 SV = 1 - | | | | | | | | |
| | [A0A0L9VAB5_PHAAN] | | | | | | | | |
| A0A0L9VAK7 | Uncharacterized protein | 7.34 | 8 | 3 | 3 | 3 | 627 | 68.2 | 6.37 |
| | OS = Phaseolus angularis | | | | | | | | |
| | GN = LR48_Vigan09g069200 | | | | | | | | |
| | PE = 4 SV = 1 - | | | | | | | | |
| | [A0A0L9VAK7_PHAAN] | | | | | | | | |
| A0A0L9VBS3 | Uncharacterized protein | 12.00 | 59 | 1 | 1 | 1 | 75 | 8.1 | 8.05 |
| | OS = Phaseolus angularis | | | | | | | | |
| | GN = LR48_Vigan09g104600 | | | | | | | | |
| | PE = 4 SV = 1 - | | | | | | | | |
| | [A0A0L9VBS3_PHAAN] | | | | | | | | |
| A0A0L9VCK7 | Uncharacterized protein | 3.08 | 4 | 1 | 1 | 1 | 357 | 37.6 | 8.09 |
| | OS = Phaseolus angularis | | | | | | | | |
| | GN = LR48_Vigan09g131100 | | | | | | | | |
| | PE = 4 SV = 1 - | | | | | | | | |
| | [A0A0L9VCK7_PHAAN] | | | | | | | | |
| A0A0L9VCC8 | Uncharacterized protein | 8.14 | 11 | 1 | 4 | 5 | 528 | 60.6 | 8.12 |
| | OS = Phaseolus angularis | | | | | | | | |
| | GN = LR48_Vigan09g138400 | | | | | | | | |
| | PE = 4 SV = 1 - | | | | | | | | |
| | [A0A0L9VCC8_PHAAN] | | | | | | | | |
| A0A0L9VD71 | Uncharacterized protein | 8.65 | 8 | 1 | 1 | 1 | 208 | 23.8 | 7.58 |
| | OS = Phaseolus angularis | | | | | | | | |
| | GN = LR48_Vigan09g159700 | | | | | | | | |
| | PE = 4 SV = 1 - | | | | | | | | |
| | [A0A0L9VD71_PHAAN] | | | | | | | | |
| A0A0L9VD76 | Uncharacterized protein | 17.52 | 11 | 1 | 2 | 3 | 137 | 15.7 | 10.64 |
| | OS = Phaseolus angularis | | | | | | | | |
| | GN = LR48_Vigan09g160200 | | | | | | | | |
| | PE = 4 SV = 1 - | | | | | | | | |
| | [A0A0L9VD76_PHAAN] | | | | | | | | |
| A0A0L9T438 | Uncharacterized protein | 5.88 | 15 | 2 | 2 | 2 | 510 | 55.2 | 7.34 |
| | OS = Phaseolus angularis | | | | | | | | |
| | GN = LR48_Vigan102s002300 | | | | | | | | |
| | PE = 4 SV = 1 - | | | | | | | | |
| | [A0A0L9T438_PHAAN] | | | | | | | | |
| A0A0L9T418 | Uncharacterized protein | 9.17 | 8 | 2 | 2 | 2 | 218 | 24.3 | 6.23 |
| | OS = Phaseolus angularis | | | | | | | | |
| | GN = LR48_Vigan107s000700 | | | | | | | | |
| | PE = 4 SV = 1 - | | | | | | | | |
| | [A0A0L9T4I8_PHAAN] | | | | | | | | |
| A0A0L9VHL1 | Uncharacterized protein | 4.58 | 36 | 1 | 2 | 2 | 589 | 64.8 | 6.04 |
| | OS = Phaseolus angularis | | | | | | | | |
| | GN = LR48_Vigan10g008600 | | | | | | | | |
| | PE = 4 SV = 1 - | | | | | | | | |
| | [A0A0L9VHL1_PHAAN] | | | | | | | | |
| A0A0L9VH79 | Uncharacterized protein | 31.93 | 12 | 2 | 3 | 3 | 119 | 13.5 | 9.45 |
| | OS = Phaseolus angularis | | | | | | | | |
| | GN = LR48_Vigan10g029800 | | | | | | | | |
| | PE = 4 SV = 1 - | | | | | | | | |
| | [A0A0L9VH79_PHAAN] | | | | | | | | |
| A0A0L9VJF0 | Uncharacterized protein | 9.87 | 6 | 1 | 6 | 8 | 699 | 80.3 | 5.08 |
| | OS = Phaseolus angularis | | | | | | | | |
| | GN = LR48_Vigan10g106800 | | | | | | | | |
| | PE = 4 SV = 1 - | | | | | | | | |
| | [A0A0L9VJF0_PHAAN] | | | | | | | | |
| A0A0L9VKF2 | Uncharacterized protein | 1.52 | 9 | 1 | 1 | 1 | 525 | 56.3 | 5.52 |
| | OS = Phaseolus angularis | | | | | | | | |
| | GN = LR48_Vigan10g141700 | | | | | | | | |
| | PE = 4 SV = 1 - | | | | | | | | |
| | [A0A0L9VKF2_PHAAN] | | | | | | | | |
| A0A0L9VM20 | Uncharacterized protein | 22.37 | 53 | 2 | 8 | 17 | 447 | 49.3 | 9.06 |
| | OS = Phaseolus angularis | | | | | | | | |
| | GN = LR48_Vigan10g199800 | | | | | | | | |
| | PE = 4 SV = 1 - | | | | | | | | |
| | [A0A0L9VM20_PHAAN] | | | | | | | | |
| A0A0L9VN25 | Uncharacterized protein | 10.88 | 8 | 1 | 6 | 10 | 763 | 84.3 | 6.39 |
| | OS = Phaseolus angularis | | | | | | | | |
| | GN = LR48_Vigan10g220400 | | | | | | | | |
| | PE = 4 SV = 1 - | | | | | | | | |
| | [A0A0L9VN25_PHAAN] | | | | | | | | |
| A0A0L9VNQ8 | Uncharacterized protein | 5.25 | 1 | 1 | 1 | 1 | 438 | 47.8 | 5.39 |
| | OS = Phaseolus angularis | | | | | | | | |
| | GN = LR48_Vigan10g227100 | | | | | | | | |
| | PE = 4 SV = 1 - | | | | | | | | |
| | [A0A0L9VNQ8_PHAAN] | | | | | | | | |
| A0A0L9T5D1 | Uncharacterized protein | 23.49 | 13 | 1 | 3 | 3 | 166 | 17.8 | 8.70 |
| | OS = Phaseolus angularis | | | | | | | | |
| | GN = LR48_Vigan115s000600 | | | | | | | | |
| | PE = 4 SV = 1 - | | | | | | | | |
| | [A0A0L9T5D1_PHAAN] | | | | | | | | |
| A0A0L9VQV7 | Uncharacterized protein | 22.81 | 24 | 3 | 3 | 4 | 171 | 19.4 | 9.32 |
| | OS = Phaseolus angularis | | | | | | | | |
| | GN = LR48_Vigan11g017100 | | | | | | | | |
| | PE = 4 SV = 1 - | | | | | | | | |
| | [A0A0L9VQV7_PHAAN] | | | | | | | | |
| A0A0L9VQL8 | Uncharacterized protein | 2.85 | 88 | 2 | 2 | 2 | 806 | 89.3 | 6.20 |
| | OS = Phaseolus angularis | | | | | | | | |
| | GN = LR48_Vigan11g018900 | | | | | | | | |
| | PE = 4 SV = 1 - | | | | | | | | |
| | [A0A0L9VQL8_PHAAN] | | | | | | | | |
| A0A0L9VS83 | Uncharacterized protein | 34.29 | 16 | 4 | 4 | 5 | 140 | 16.0 | 10.61 |
| | OS = Phaseolus angularis | | | | | | | | |
| | GN = LR48_Vigan11g076500 | | | | | | | | |
| | PE = 4 SV = 1 - | | | | | | | | |
| | [A0A0L9VS83_PHAAN] | | | | | | | | |
| A0A0L9VUQ7 | Uncharacterized protein | 7.14 | 3 | 1 | 1 | 1 | 112 | 11.6 | 5.07 |
| | OS = Phaseolus angularis | | | | | | | | |
| | GN = LR48_Vigan11g160100 | | | | | | | | |
| | PE = 4 SV = 1 - | | | | | | | | |
| | [A0A0L9VUQ7_PHAAN] | | | | | | | | |
| A0A0L9T2U7 | Uncharacterized protein | 4.34 | 8 | 1 | 1 | 1 | 392 | 43.2 | 5.62 |
| | OS = Phaseolus angularis | | | | | | | | |
| | GN = LR48_Vigan17s000600 | | | | | | | | |
| | PE = 4 SV = 1 - | | | | | | | | |
| | [A0A0L9T2U7_PHAAN] | | | | | | | | |
| A0A0L9T5L6 | Uncharacterized protein | 12.42 | 48 | 1 | 4 | 4 | 443 | 49.7 | 4.84 |
| | OS = Phaseolus angularis | | | | | | | | |
| | GN = LR48_Vigan205s003200 | | | | | | | | |
| | PE = 4 SV = 1 - | | | | | | | | |
| | [A0A0L9T5L6_PHAAN] | | | | | | | | |
| A0A0L9T5Q5 | Uncharacterized protein | 9.24 | 7 | 1 | 1 | 1 | 184 | 21.2 | 5.39 |
| | OS = Phaseolus angularis | | | | | | | | |
| | GN = LR48_Vigan205s004400 | | | | | | | | |
| | PE = 4 SV = 1 - | | | | | | | | |
| | [A0A0L9T5Q5_PHAAN] | | | | | | | | |
| A0A0L9T7H3 | Uncharacterized protein | 1.23 | 1 | 1 | 1 | 1 | 1217 | 148.3 | 4.42 |
| | OS = Phaseolus angularis | | | | | | | | |
| | GN = LR48_Vigan221s001600 | | | | | | | | |
| | PE = 4 SV = 1 - | | | | | | | | |
| | [A0A0L9T7H3_PHAAN] | | | | | | | | |
| A0A0L9T6F7 | Uncharacterized protein | 9.27 | 5 | 2 | 2 | 2 | 248 | 26.3 | 7.21 |
| | OS = Phaseolus angularis | | | | | | | | |
| | GN = LR48_Vigan238s001300 | | | | | | | | |
| | PE = 4 SV = 1 - | | | | | | | | |
| | [A0A0L9T6F7_PHAAN] | | | | | | | | |
| A0A0L9TA18 | Uncharacterized protein | 16.32 | 42 | 0 | 8 | 11 | 576 | 62.9 | 5.25 |
| | OS = Phaseolus angularis | | | | | | | | |
| | GN = LR48_Vigan406s003600 | | | | | | | | |
| | PE = 4 SV = 1 - | | | | | | | | |
| | [A0A0L9TA18_PHAAN] | | | | | | | | |
| A0A0L9T9T3 | Uncharacterized protein | 10.27 | 4 | 1 | 1 | 2 | 146 | 16.3 | 5.81 |
| | OS = Phaseolus angularis | | | | | | | | |
| | GN = LR48_Vigan406s012700 | | | | | | | | |
| | PE = 4 SV = 1 - | | | | | | | | |
| | [A0A0L9T9T3_PHAAN] | | | | | | | | |
| A0A0L9TBY3 | Uncharacterized protein | 20.31 | 9 | 1 | 4 | 5 | 261 | 28.2 | 10.81 |
| | OS = Phaseolus angularis | | | | | | | | |
| | GN = LR48_Vigan499s000200 | | | | | | | | |
| | PE = 4 SV = 1 - | | | | | | | | |
| | [A0A0L9TBY3_PHAAN] | | | | | | | | |
| A0A0L9TEC9 | Uncharacterized protein | 12.20 | 2 | 2 | 2 | 2 | 295 | 31.4 | 6.02 |
| | OS = Phaseolus angularis | | | | | | | | |
| | GN = LR48_Vigan549s002900 | | | | | | | | |
| | PE = 4 SV = 1 - | | | | | | | | |
| | [A0A0L9TEC9_PHAAN] | | | | | | | | |
| A0A0L9TDA7 | Uncharacterized protein | 3.70 | 7 | 2 | 2 | 2 | 513 | 56.6 | 5.55 |
| | OS = Phaseolus angularis | | | | | | | | |
| | GN = LR48_Vigan549s004700 | | | | | | | | |
| | PE = 4 SV = 1 - | | | | | | | | |
| | [A0A0L9TDA7_PHAAN] | | | | | | | | |
| A0A0L9TE86 | Uncharacterized protein | 6.57 | 17 | 3 | 3 | 4 | 548 | 58.4 | 5.74 |
| | OS = Phaseolus angularis | | | | | | | | |
| | GN = LR48_Vigan598s001900 | | | | | | | | |
| | PE = 4 SV = 1 - | | | | | | | | |
| | [A0A0L9TE86_PHAAN] | | | | | | | | |
| A0A0L9TFH9 | Uncharacterized protein | 32.62 | 107 | 4 | 12 | 17 | 558 | 59.6 | 6.29 |
| | OS = Phaseolus angularis | | | | | | | | |
| | GN = LR48_Vigan609s001100 | | | | | | | | |
| | PE = 4 SV = 1 - | | | | | | | | |
| | [A0A0L9TFH9_PHAAN] | | | | | | | | |
| A0A0L9TEM8 | Uncharacterized protein | 1.26 | 1 | 1 | 1 | 2 | 635 | 70.6 | 5.99 |
| | OS = Phaseolus angularis | | | | | | | | |
| | GN = LR48_Vigan627s007200 | | | | | | | | |
| | PE = 4 SV = 1 - | | | | | | | | |
| | [A0A0L9TEM8_PHAAN] | | | | | | | | |
| A0A0L9TF36 | Uncharacterized protein | 9.59 | 1 | 1 | 1 | 2 | 146 | 16.1 | 5.22 |
| | OS = Phaseolus angularis | | | | | | | | |
| | GN = LR48_Vigan641s001300 | | | | | | | | |
| | PE = 4 SV = 1 - | | | | | | | | |
| | [A0A0L9TF36_PHAAN] | | | | | | | | |
| A0A0L9TI04 | Uncharacterized protein | 7.56 | 11 | 1 | 4 | 6 | 582 | 63.4 | 5.48 |
| | OS = Phaseolus angularis | | | | | | | | |
| | GN = LR48_Vigan847s000500 | | | | | | | | |
| | PE = 4 SV = 1 - | | | | | | | | |
| | [A0A0L9TI04_PHAAN] | | | | | | | | |
| V7CUH6 | Uncharacterized protein | 5.18 | 6 | 1 | 3 | 3 | 541 | 60.3 | 6.37 |
| | OS = Phaseolus vulgaris | | | | | | | | |
| | GN = PHAVU_001G030000g | | | | | | | | |
| | PE = 3 SV = 1 - | | | | | | | | |
| | [V7CUH6_PHAVU] | | | | | | | | |
| V7CX68 | Uncharacterized protein | 17.96 | 14 | 0 | 10 | 16 | 763 | 84.0 | 6.35 |
| | OS = Phaseolus vulgaris | | | | | | | | |
| | GN = PHAVU_001G113800g | | | | | | | | |
| | PE = 3 SV = 1 - | | | | | | | | |
| | [V7CX68_PHAVU] | | | | | | | | |
| V7D0I8 | Uncharacterized protein | 17.68 | 13 | 3 | 3 | 3 | 164 | 18.7 | 10.52 |
| | OS = Phaseolus vulgaris | | | | | | | | |
| | GN = PHAVU_001G201000g | | | | | | | | |
| | PE = 4 SV = 1 - | | | | | | | | |
| | [V7D0I8_PHAVU] | | | | | | | | |
| V7D286 | Uncharacterized protein | 12.75 | 31 | 2 | 2 | 2 | 149 | 16.9 | 5.35 |
| | OS = Phaseolus vulgaris | | | | | | | | |
| | GN = PHAVU_001G225400g | | | | | | | | |
| | PE = 4 SV = 1 - | | | | | | | | |
| | [V7D286_PHAVU] | | | | | | | | |
| V7CZG7 | Uncharacterized protein | 7.26 | 1 | 2 | 2 | 3 | 317 | 33.9 | 5.41 |
| | OS = Phaseolus vulgaris | | | | | | | | |
| | GN = PHAVU_001G241400g | | | | | | | | |
| | PE = 3 SV = 1 - | | | | | | | | |
| | [V7CZG7_PHAVU] | | | | | | | | |
| V7CH59 | Uncharacterized protein | 3.45 | 1 | 1 | 1 | 1 | 435 | 47.7 | 5.20 |
| | OS = Phaseolus vulgaris | | | | | | | | |
| | GN = PHAVU_002G007200g | | | | | | | | |
| | PE = 4 SV = 1 - | | | | | | | | |
| | [V7CH59_PHAVU] | | | | | | | | |
| V7C7A9 | Uncharacterized protein | 2.33 | 1 | 1 | 1 | 1 | 687 | 77.1 | 6.95 |
| | OS = Phaseolus vulgaris | | | | | | | | |
| | GN = PHAVU_003G019700g | | | | | | | | |
| | PE = 4 SV = 1 - | | | | | | | | |
| | [V7C7A9_PHAVU] | | | | | | | | |
| V7C961 | Uncharacterized protein | 6.19 | 10 | 1 | 2 | 2 | 420 | 47.8 | 6.32 |
| | OS = Phaseolus vulgaris | | | | | | | | |
| | GN = PHAVU_003G075200g | | | | | | | | |
| | PE = 4 SV = 1 - | | | | | | | | |
| | [V7C961_PHAVU] | | | | | | | | |
| V7CAX0 | Uncharacterized protein | 4.93 | 7 | 1 | 1 | 1 | 284 | 31.3 | 6.32 |
| | OS = Phaseolus vulgaris | | | | | | | | |
| | GN = PHAVU_003G192700g | | | | | | | | |
| | PE = 4 SV = 1 - | | | | | | | | |
| | [V7CAX0_PHAVU] | | | | | | | | |
| V7BZE4 | Uncharacterized protein | 6.08 | 3 | 1 | 1 | 1 | 181 | 20.4 | 8.73 |
| | OS = Phaseolus vulgaris | | | | | | | | |
| | GN = PHAVU_004G031600g | | | | | | | | |
| | PE = 4 SV = 1 - | | | | | | | | |
| | [V7BZE4_PHAVU] | | | | | | | | |
| V7C2J2 | Uncharacterized protein | 6.79 | 11 | 1 | 1 | 2 | 162 | 17.6 | 8.31 |
| | OS = Phaseolus vulgaris | | | | | | | | |
| | GN = PHAVU_004G060100g | | | | | | | | |
| | PE = 3 SV = 1 - | | | | | | | | |
| | [V7C2J2_PHAVU] | | | | | | | | |
| V7BXI7 | Uncharacterized protein | 15.64 | 19 | 2 | 11 | 11 | 831 | 92.5 | 6.02 |
| | OS = Phaseolus vulgaris | | | | | | | | |
| | GN = PHAVU_005G058100g | | | | | | | | |
| | PE = 4 SV = 1 - | | | | | | | | |
| | [V7BXI7_PHAVU] | | | | | | | | |
| V7BPB1 | Uncharacterized protein | 6.18 | 2 | 2 | 2 | 2 | 518 | 55.9 | 6.62 |
| | OS = Phaseolus vulgaris | | | | | | | | |
| | GN = PHAVU_006G079500g | | | | | | | | |
| | PE = 4 SV = 1 - | | | | | | | | |
| | [V7BPB1_PHAVU] | | | | | | | | |
| V7BN55 | Uncharacterized protein | 10.36 | 6 | 2 | 2 | 15 | 309 | 33.6 | 8.44 |
| | OS = Phaseolus vulgaris | | | | | | | | |
| | GN = PHAVU_006G120100g | | | | | | | | |
| | PE = 4 SV = 1 - | | | | | | | | |
| | [V7BN55_PHAVU] | | | | | | | | |
| V7BNA3 | Uncharacterized protein | 1.85 | 1 | 1 | 1 | 1 | 540 | 60.7 | 9.16 |
| | OS = Phaseolus vulgaris | | | | | | | | |
| | GN = PHAVU_006G124600g | | | | | | | | |
| | PE = 4 SV = 1 - | | | | | | | | |
| | [V7BNA3_PHAVU] | | | | | | | | |
| V7B9Z8 | Uncharacterized protein | 2.76 | 9 | 1 | 1 | 1 | 435 | 47.6 | 7.21 |
| | OS = Phaseolus vulgaris | | | | | | | | |
| | GN = PHAVU_007G004600g | | | | | | | | |
| | PE = 3 SV = 1 - | | | | | | | | |
| | [V7B9Z8_PHAVU] | | | | | | | | |
| V7BEF1 | Uncharacterized protein | 1.26 | 2 | 1 | 1 | 1 | 556 | 65.0 | 8.63 |
| | OS = Phaseolus vulgaris | | | | | | | | |
| | GN = PHAVU_007G059800g | | | | | | | | |
| | PE = 4 SV = 1 - | | | | | | | | |
| | [V7BEF1_PHAVU] | | | | | | | | |
| V7BE66 | Uncharacterized protein | 10.97 | 8 | 1 | 2 | 5 | 237 | 26.9 | 6.34 |
| | OS = Phaseolus vulgaris | | | | | | | | |
| | GN = PHAVU_007G130900g | | | | | | | | |
| | PE = 4 SV = 1 - | | | | | | | | |
| | [V7BE66_PHAVU] | | | | | | | | |
| V7BHN0 | Uncharacterized protein | 4.37 | 14 | 2 | 2 | 2 | 458 | 48.3 | 7.81 |
| | OS = Phaseolus vulgaris | | | | | | | | |
| | GN = PHAVU 007G236600g | | | | | | | | |
| | PE = 3 SV = 1 - | | | | | | | | |
| | [V7BHN0_PHAVU] | | | | | | | | |
| V7B0A5 | Uncharacterized protein | 23.11 | 80 | 2 | 13 | 20 | 649 | 71.1 | 5.25 |
| | OS = Phaseolus vulgaris | | | | | | | | |
| | GN = PHAVU_008G013000g | | | | | | | | |
| | PE = 3 SV = 1 - | | | | | | | | |
| | [V7B0A5_PHAVU] | | | | | | | | |
| V7B980 | Uncharacterized protein | 3.86 | 1 | 1 | 1 | 1 | 492 | 53.9 | 5.41 |
| | OS = Phaseolus vulgaris | | | | | | | | |
| | GN = PHAVU_008G194300g | | | | | | | | |
| | PE = 4 SV = 1 - | | | | | | | | |
| | [V7B980_PHAVU] | | | | | | | | |
| V7B7F8 | Uncharacterized protein | 12.81 | 11 | 1 | 3 | 3 | 320 | 35.7 | 4.93 |
| | OS = Phaseolus vulgaris | | | | | | | | |
| | GN = PHAVU_008G203600g | | | | | | | | |
| | PE = 4 SV = 1 - | | | | | | | | |
| | [V7B7F8_PHAVU] | | | | | | | | |
| V7BD88 | Uncharacterized protein | 14.47 | 29 | 2 | 9 | 10 | 698 | 80.0 | 5.06 |
| | OS = Phaseolus vulgaris | | | | | | | | |
| | GN = PHAVU_008G281400g | | | | | | | | |
| | PE = 3 SV = 1 - | | | | | | | | |
| | [V7BD88_PHAVU] | | | | | | | | |
| V7ASY8 | Uncharacterized protein | 4.62 | 9 | 1 | 1 | 1 | 260 | 29.1 | 10.46 |
| | OS = Phaseolus vulgaris | | | | | | | | |
| | GN = PHAVU_009G046000g | | | | | | | | |
| | PE = 4 SV = 1 - | | | | | | | | |
| | [V7ASY8_PHAVU] | | | | | | | | |
| V7AW79 | Uncharacterized protein | 4.52 | 1 | 1 | 1 | 1 | 310 | 33.8 | 6.89 |
| | OS = Phaseolus vulgaris | | | | | | | | |
| | GN = PHAVU_009G049300g | | | | | | | | |
| | PE = 4 SV = 1 - | | | | | | | | |
| | [V7AW79_PHAVU] | | | | | | | | |
| V7ATV2 | Uncharacterized protein | 5.49 | 27 | 1 | 1 | 1 | 164 | 18.3 | 5.54 |
| | OS = Phaseolus vulgaris | | | | | | | | |
| | GN = PHAVU_009G069500g | | | | | | | | |
| | PE = 4 SV = 1 - | | | | | | | | |
| | [V7ATV2_PHAVU] | | | | | | | | |
| V7B2J8 | Uncharacterized protein | 2.59 | 2 | 1 | 1 | 1 | 424 | 45.4 | 5.36 |
| | OS = Phaseolus vulgaris | | | | | | | | |
| | GN = PHAVU_009G226700g | | | | | | | | |
| | PE = 3 SV = 1 - | | | | | | | | |
| | [V7B2J8_PHAVU] | | | | | | | | |
| V7AKX1 | Uncharacterized protein | 8.83 | 2 | 1 | 4 | 4 | 521 | 58.2 | 7.49 |
| | OS = Phaseolus vulgaris | | | | | | | | |
| | GN = PHAVU_010G024900g | | | | | | | | |
| | PE = 3 SV = 1 - | | | | | | | | |
| | [V7AKX1_PHAVU] | | | | | | | | |
| V7AE59 | Uncharacterized protein | 1.80 | 3 | 1 | 1 | 1 | 556 | 61.4 | 8.91 |
| | OS = Phaseolus vulgaris | | | | | | | | |
| | GN = PHAVU_011G042200g | | | | | | | | |
| | PE = 4 SV = 1 - | | | | | | | | |
| | [V7AE59_PHAVU] | | | | | | | | |
| V7AEY8 | Uncharacterized protein | 1.60 | 5 | 1 | 1 | 1 | 500 | 57.4 | 8.21 |
| | OS = Phaseolus vulgaris | | | | | | | | |
| | GN = PHAVU_011G045200g | | | | | | | | |
| | PE = 3 SV = 1 - | | | | | | | | |
| | [V7AEY8_PHAVU] | | | | | | | | |
| V7AHB3 | Uncharacterized protein | 7.83 | 32 | 2 | 2 | 2 | 332 | 35.4 | 8.82 |
| | OS = Phaseolus vulgaris | | | | | | | | |
| | GN = PHAVU_011G077900g | | | | | | | | |
| | PE = 3 SV = 1 - | | | | | | | | |
| | [V7AHB3_PHAVU] | | | | | | | | |
| A0A0B2QZE2 | UPF0664 stress-induced | 5.34 | 6 | 1 | 1 | 1 | 206 | 23.3 | 7.11 |
| | protein C29B12.11c | | | | | | | | |
| | OS = Glycine soja | | | | | | | | |
| | GN = glysoja_050003 PE = 4 | | | | | | | | |
| | SV = 1 - | | | | | | | | |
| | [A0A0B2QZE2_GLYSO] | | | | | | | | |
| Q9FEH0 | Uricase (Fragment) | 3.90 | 2 | 1 | 1 | 1 | 205 | 23.2 | 9.01 |
| | OS = Astragalus sinicus | | | | | | | | |
| | GN = AsUr9 PE = 2 SV = 1 - | | | | | | | | |
| | [Q9FEH0_ASTSI] | | | | | | | | |
| Q9FRY5 | Uricase (Fragment) | 3.90 | 7 | 1 | 1 | 1 | 205 | 23.2 | 9.22 |
| | OS = Pisum sativum | | | | | | | | |
| | GN = PsUr PE = 2 SV = 1 - | | | | | | | | |
| | [Q9FRY5_PEA] | | | | | | | | |
| A0A0B2RYF1 | UTP--glucose-1-phosphate | 30.97 | 20 | 2 | 12 | 19 | 507 | 56.0 | 6.25 |
| | uridylyltransferase | | | | | | | | |
| | OS = Glycine soja | | | | | | | | |
| | GN = glysoja_016883 PE = 4 | | | | | | | | |
| | SV = 1 - | | | | | | | | |
| | [A0A0B2RYF1_GLYSO] | | | | | | | | |
| Q4TUS1 | Vacuolar H+-ATPase | 3.47 | 9 | 1 | 1 | 1 | 375 | 41.2 | 5.52 |
| | subunit A (Fragment) | | | | | | | | |
| | OS = Vigna unguiculata | | | | | | | | |
| | PE = 2 SV = 1 - | | | | | | | | |
| | [Q4TUS1_VIGUN] | | | | | | | | |
| A0A0B2S5M2 | Vicilin-like antimicrobial | 2.16 | 2 | 1 | 1 | 1 | 602 | 70.9 | 5.26 |
| | peptides 2-2 OS = Glycine | | | | | | | | |
| | soja GN = glysoja_003741 | | | | | | | | |
| | PE = 4 SV = 1 - | | | | | | | | |
| | [A0A0B2S5M2_GLYSO] | | | | | | | | |
| A0A068LJH6 | Vitamin B-12-independent | 12.32 | 8 | 1 | 8 | 10 | 763 | 84.3 | 6.42 |
| | methionine synthase | | | | | | | | |
| | OS = Pisum sativum PE = 2 | | | | | | | | |
| | SV = 1 - | | | | | | | | |
| | [A0A068LJH6_PEA] | | | | | | | | |
| B7FJF0 | Xylose isomerase | 4.86 | 3 | 2 | 2 | 2 | 329 | 36.8 | 6.02 |
| | OS = Medicago truncatula | | | | | | | | |
| | PE = 2 SV = 1 - | | | | | | | | |
| | [B7FJF0_MEDTR] | | | | | | | | |

A Total of 490 new found proteins in Cashew and their sequence

| | | | # | # Unique | # | # | | MW | calc. |
|---|---|---|---|---|---|---|---|---|---|
| Accession | Description | Coverage | Proteins | Peptides | Peptides | PSMs | # AAs | [kDa] | pI |
| A0A088SFK8 | 11S globulin OS = Murraya | 1.69 | 3 | 1 | 1 | 2 | 474 | 53.0 | 7.06 |
| | paniculata PE = 2 SV = 1 - | | | | | | | | |
| | [A0A088SFK8_9ROSI] | | | | | | | | |
| B2KN55 | 11S globulin OS = Pistacia | 10.81 | 2 | 3 | 4 | 32 | 472 | 53.2 | 7.24 |
| | vera PE = 2 SV = 1 - | | | | | | | | |
| | [B2KN55_PISVE] | | | | | | | | |
| D3TIB7 | 14-3-3 (Fragment) | 23.75 | 18 | 2 | 5 | 9 | 261 | 29.3 | 4.78 |
| | OS = Mangifera indica PE = 2 | | | | | | | | |
| | SV = 1 - [D3TIB7_MANIN] | | | | | | | | |
| Q8H2B8 | 2s albumin OS = Anacardium | 50.00 | 1 | 9 | 9 | 53 | 138 | 16.3 | 5.68 |
| | occidentale GN = ana o 3 | | | | | | | | |
| | PE = 2 SV = 1 - | | | | | | | | |
| | [Q8H2B8_ANAOC] | | | | | | | | |
| V4SJG0 | 3-isopropylmalate | 6.63 | 2 | 2 | 2 | 2 | 407 | 43.5 | 6.15 |
| | dehydrogenase OS = Citrus | | | | | | | | |
| | clementina | | | | | | | | |
| | GN = CICLE_v10025744mg | | | | | | | | |
| | PE = 3 SV = 1 - | | | | | | | | |
| | [V4SJG0_9ROSI] | | | | | | | | |
| V4SPM1 | 40S ribosomal protein S12 | 19.15 | 1 | 2 | 2 | 3 | 141 | 14.8 | 6.27 |
| | OS = Citrus clementina | | | | | | | | |
| | GN = CICLE_v10026727mg | | | | | | | | |
| | PE = 3 SV = 1 - | | | | | | | | |
| | [V4SPM1_9ROSI] | | | | | | | | |
| V45F69 | 40S ribosomal protein S24 | 17.91 | 1 | 2 | 2 | 2 | 134 | 15.4 | 10.70 |
| | OS = Citrus clementina | | | | | | | | |
| | GN = CICLE_v10002819mg | | | | | | | | |
| | PE = 3 SV = 1 - | | | | | | | | |
| | [V4SF69_9ROSI] | | | | | | | | |
| V4W2J8 | 40S ribosomal protein S27 | 29.07 | 2 | 2 | 2 | 2 | 86 | 9.6 | 8.73 |
| | OS = Citrus clementina | | | | | | | | |
| | GN = CICLE_v10017332mg | | | | | | | | |
| | PE = 3 SV = 1 - | | | | | | | | |
| | [V4W2J8_9ROSI] | | | | | | | | |
| A0A067G4F6 | 40S ribosomal protein S3a | 9.39 | 2 | 2 | 2 | 2 | 245 | 28.2 | 9.99 |
| | OS = Citrus sinensis | | | | | | | | |
| | GN = CISIN_1g024879mg | | | | | | | | |
| | PE = 3 SV = 1 - | | | | | | | | |
| | [A0A067G4F6_CITSI] | | | | | | | | |
| A0A067FUW4 | 40S ribosomal protein S4 | 19.43 | 9 | 5 | 5 | 6 | 247 | 27.9 | 10.10 |
| | OS = Citrus sinensis | | | | | | | | |
| | GN = CISIN_1g024108mg | | | | | | | | |
| | PE = 3 SV = 1 - | | | | | | | | |
| | [A0A067FUW4_CITSI] | | | | | | | | |
| V4SG60 | 40S ribosomal protein S6 | 11.65 | 4 | 3 | 3 | 3 | 249 | 28.4 | 10.64 |
| | OS = Citrus clementina | | | | | | | | |
| | GN = CICLE_v10026340mg | | | | | | | | |
| | PE = 3 SV = 1 - | | | | | | | | |
| | [V4SG60_9ROSI] | | | | | | | | |
| A0A067D033 | 40S ribosomal protein S8 | 23.50 | 4 | 4 | 4 | 4 | 217 | 24.7 | 10.39 |
| | (Fragment) OS = Citrus | | | | | | | | |
| | sinensis | | | | | | | | |
| | GN = CISIN_1g044642mg | | | | | | | | |
| | PE = 3 SV = 1 - | | | | | | | | |
| | [A0A067D033_CITSI] | | | | | | | | |
| A0A067E4D6 | 40S ribosomal protein SA | 5.08 | 5 | 2 | 2 | 2 | 315 | 34.5 | 5.83 |
| | OS = Citrus sinensis | | | | | | | | |
| | GN = CISIN_1g018448mg | | | | | | | | |
| | PE = 3 SV = 1 - | | | | | | | | |
| | [A0A067E4D6_CITSI] | | | | | | | | |
| A0A067EJI8 | 4-hydroxy-4-methyl-2-oxoglutarate | 11.45 | 1 | 2 | 2 | 2 | 166 | 17.7 | 5.57 |
| | aldolase OS = Citrus | | | | | | | | |
| | sinensis | | | | | | | | |
| | GN = CISIN_1g031057mg | | | | | | | | |
| | PE = 3 SV = 1 - | | | | | | | | |
| | [A0A067EJI8_CITSI] | | | | | | | | |
| V4UL33 | 60S ribosomal protein L13 | 9.22 | 3 | 2 | 2 | 2 | 206 | 23.7 | 10.84 |
| | OS = Citrus clementina | | | | | | | | |
| | GN = CICLE_v10009507mg | | | | | | | | |
| | PE = 3 SV = 1 - | | | | | | | | |
| | [V4UL33_9ROSI] | | | | | | | | |
| A0A067E9K7 | 60S ribosomal protein L18a | 16.67 | 6 | 3 | 3 | 3 | 180 | 21.7 | 10.52 |
| | OS = Citrus sinensis | | | | | | | | |
| | GN = CISIN_1g030276mg | | | | | | | | |
| | PE = 3 SV = 1 - | | | | | | | | |
| | [A0A067E9K7_CITSI] | | | | | | | | |
| V4TH53 | 6-phosphogluconate | 7.09 | 9 | 2 | 3 | 3 | 494 | 54.2 | 6.70 |
| | dehydrogenase, | | | | | | | | |
| | decarboxylating OS = Citrus | | | | | | | | |
| | clementina | | | | | | | | |
| | GN = CICLE_v10019859mg | | | | | | | | |
| | PE = 3 SV = 1 - | | | | | | | | |
| | [V4TH53_9ROSI] | | | | | | | | |
| A0A067G3F9 | 6-phosphogluconate | 7.23 | 8 | 2 | 3 | 3 | 484 | 53.5 | 6.81 |
| | dehydrogenase, | | | | | | | | |
| | decarboxylating OS = Citrus | | | | | | | | |
| | sinensis | | | | | | | | |
| | GN = CISIN_1g0115012mg | | | | | | | | |
| | PE = 3 SV = 1 - | | | | | | | | |
| | [A0A067G3F9_CITSI] | | | | | | | | |
| A0A067YQD1 | Acetyl-coenzyme A | 3.85 | 2 | 2 | 2 | 2 | 493 | 55.9 | 5.87 |
| | carboxylase carboxyl | | | | | | | | |
| | transferase subunit beta, | | | | | | | | |
| | chloroplastic OS = Acer | | | | | | | | |
| | buergerianum subsp. | | | | | | | | |
| | ningpoense GN = accD PE = 3 | | | | | | | | |
| | SV = 1 - | | | | | | | | |
| | [A0A067YQD1_ACEBU] | | | | | | | | |
| A0A067FPP5 | Acetyltransferase component | 5.18 | 6 | 2 | 2 | 2 | 425 | 46.0 | 5.73 |
| | of pyruvate dehydrogenase | | | | | | | | |
| | complex OS = Citrus sinensis | | | | | | | | |
| | GN = CISIN_1g008996mg | | | | | | | | |
| | PE = 3 SV = 1 - | | | | | | | | |
| | [A0A067FPP5_CITSI] | | | | | | | | |
| F2Y9D8 | Aconitate hydratase | 16.87 | 3 | 2 | 9 | 9 | 883 | 96.5 | 6.06 |
| | (Fragment) OS = Litchi | | | | | | | | |
| | chinensis PE = 2 SV = 1 - | | | | | | | | |
| | [F2Y9D8_LITCN] | | | | | | | | |
| D3GQL2 | Aconitate hydratase | 17.26 | 14 | 2 | 10 | 11 | 898 | 97.8 | 6.54 |
| | OS = Citrus clementina | | | | | | | | |
| | GN = aco2 PE = 2 SV = 1 - | | | | | | | | |
| | [D3GQL2_9ROSI] | | | | | | | | |
| A0A067FYY8 | Aconitate hydratase | 13.05 | 13 | 4 | 8 | 9 | 889 | 97.2 | 6.54 |
| | OS = Citrus sinensis | | | | | | | | |
| | GN = CISIN_1g002610mg | | | | | | | | |
| | PE = 3 SV = 1 - | | | | | | | | |
| | [A0A067FYY8_CITSI] | | | | | | | | |
| F2XI27 | Actin OS = Litchi chinensis | 36.08 | 2 | 1 | 9 | 11 | 316 | 34.9 | 5.11 |
| | GN = ACT7 PE = 2 SV = 1 - | | | | | | | | |
| | [F2XI27_LITCN] | | | | | | | | |
| E9RTU0 | Actin OS = Nitraria sibirica | 46.15 | 13 | 3 | 12 | 18 | 377 | 41.7 | 5.49 |
| | GN = act PE = 2 SV = 1 - | | | | | | | | |
| | [E9RJU0_9ROSI] | | | | | | | | |
| A0A067FL83 | Acyl-[acyl-carrier-protein] | 6.31 | 4 | 2 | 2 | 2 | 396 | 45.3 | 6.48 |
| | desaturase OS = Citrus | | | | | | | | |
| | sinensis | | | | | | | | |
| | GN = CISIN_1g016050mg | | | | | | | | |
| | PE = 3 SV = 1 - | | | | | | | | |
| | [A0A067FL83_CITSI] | | | | | | | | |
| A0A067DJQ6 | Adenosylhomocysteinase | 10.31 | 5 | 5 | 5 | 5 | 485 | 53.1 | 6.10 |
| | OS = Citrus sinensis | | | | | | | | |
| | GN = CISIN_1g011464mg | | | | | | | | |
| | PE = 3 SV = 1 - | | | | | | | | |
| | [A0A067DJQ6_CITSI] | | | | | | | | |
| D2XQZ8 | Alcohol dehydrogenase 1 | 22.77 | 15 | 4 | 7 | 9 | 382 | 41.8 | 6.46 |
| | OS = Mangifera indica PE = 2 | | | | | | | | |
| | SV = 1 - [D2XQZ8_MANIN] | | | | | | | | |
| Q4JIY8 | Alcohol dehydrogenase | 13.95 | 22 | 1 | 4 | 7 | 380 | 41.3 | 6.20 |
| | OS = Citrus paradisi PE = 2 | | | | | | | | |
| | SV = 1 - [Q4JIY8_CITPA] | | | | | | | | |
| Q8GZP6 | Allergen Ana o 2 (Fragment) | 71.12 | 1 | 34 | 35 | 527 | 457 | 52.0 | 6.61 |
| | OS = Anacardium occidentale | | | | | | | | |
| | PE = 2 SV = 1 - | | | | | | | | |
| | [Q8GZP6_ANAOC] | | | | | | | | |
| V4SAE0 | Alpha-1,4 glucan | 8.09 | 9 | 3 | 6 | 10 | 1001 | 112.8 | 4.92 |
| | phosphorylase OS = Citrus | | | | | | | | |
| | clementina | | | | | | | | |
| | GN = CICLE_v10010975mg | | | | | | | | |
| | PE = 3 SV = 1 - | | | | | | | | |
| | [V4SAE0_9ROSI] | | | | | | | | |
| A0A0K0MBS0 | Alpha-D-mannosidase | 7.14 | 10 | 1 | 2 | 2 | 350 | 39.1 | 6.95 |
| | isoform 3(Fragment) | | | | | | | | |
| | OS = Mangifera indica PE = 2 | | | | | | | | |
| | SV = 1 - | | | | | | | | |
| | [A0A0K0MBS0_MANIN] | | | | | | | | |
| A0A067EK36 | Alpha-mannosidase | 2.38 | 7 | 1 | 2 | 2 | 883 | 100.0 | 6.28 |
| | OS = Citrus sinensis | | | | | | | | |
| | GN = CISIN_1g001813mg | | | | | | | | |
| | PE = 3 SV = 1 - | | | | | | | | |
| | [A0A067EK36_CITSI] | | | | | | | | |
| V4SW23 | Annexin OS = Citrus | 2.53 | 1 | 1 | 1 | 1 | 316 | 35.5 | 8.25 |
| | clementina | | | | | | | | |
| | GN = CICLE_v10032154mg | | | | | | | | |
| | PE = 3 SV = 1 - | | | | | | | | |
| | [V4SW23_9ROSI] | | | | | | | | |
| A0A067FIX8 | Aspartate aminotransferase | 12.46 | 3 | 4 | 4 | 4 | 337 | 37.6 | 7.78 |
| | (Fragment) OS = Citrus | | | | | | | | |
| | sinensis | | | | | | | | |
| | GN = CISIN_1g047180mg | | | | | | | | |
| | PE = 3 SV = 1 - | | | | | | | | |
| | [A0A067FIX8_CITSI] | | | | | | | | |
| V4UCV7 | Aspartate aminotransferase | 11.43 | 6 | 4 | 4 | 4 | 350 | 38.0 | 9.35 |
| | OS = Citrus clementina | | | | | | | | |
| | GN = CICLE_v10008274mg | | | | | | | | |
| | PE = 3 SV = 1 - | | | | | | | | |
| | [V4UCV7_9ROSI] | | | | | | | | |
| A0A067DVF3 | Aspartate aminotransferase | 5.51 | 4 | 1 | 1 | 1 | 363 | 39.4 | 8.91 |
| | OS = Citrus sinensis | | | | | | | | |
| | GN = CISIN_1g012238mg | | | | | | | | |
| | PE = 3 SV = 1 - | | | | | | | | |
| | [A0A067DVF3_CITSI] | | | | | | | | |
| Q5IBJ6 | ATP synthase subunit alpha | 18.87 | 13 | 8 | 8 | 9 | 424 | 45.3 | 7.36 |
| | (Fragment) OS = Aesculus | | | | | | | | |
| | californica GN = atp1 PE = 3 | | | | | | | | |
| | SV = 1 - [Q5IBJ6_9ROSI] | | | | | | | | |
| A0A067FNL4 | ATP synthase subunit beta | 19.89 | 392 | 8 | 8 | 18 | 558 | 59.7 | 6.49 |
| | OS = Citrus sinensis | | | | | | | | |
| | GN = CISIN_1g008610mg | | | | | | | | |
| | PE = 3 SV = 1 - | | | | | | | | |
| | [A0A067FNL4_CITSI] | | | | | | | | |
| A0A067E3U4 | Beta-galactosidase | 0.94 | 2 | 1 | 1 | 1 | 849 | 95.7 | 6.61 |
| | (Fragment) OS = Citrus | | | | | | | | |
| | sinensis | | | | | | | | |
| | GN = CISIN_1g038226mg | | | | | | | | |
| | PE = 3 SV = 1 - | | | | | | | | |
| | [A0A067E3U4_CITSI] | | | | | | | | |
| C0KLD1 | Beta-tubulin OS = Citrus | 10.14 | 14 | 1 | 4 | 4 | 444 | 49.8 | 4.93 |
| | maxima PE = 2 SV = 1 - | | | | | | | | |
| | [C0KLD1_CITMA] | | | | | | | | |
| V4T5X4 | Catalase OS = Citrus | 4.24 | 7 | 1 | 2 | 2 | 425 | 49.0 | 6.60 |
| | clementina | | | | | | | | |
| | GN = CICLE_v10000948mg | | | | | | | | |
| | PE = 3 SV = 1 - | | | | | | | | |
| | [V4T5X4_9ROSI] | | | | | | | | |
| V4SLF8 | Catalase OS = Citrus | 3.25 | 5 | 1 | 2 | 2 | 492 | 56.9 | 7.36 |
| | clementina | | | | | | | | |
| | GN = CICLE_v10000951mg | | | | | | | | |
| | PE = 3 SV = 1 - | | | | | | | | |
| | [V4SLF8_9ROSI] | | | | | | | | |
| G5CBR3 | Citrate synthase (Fragment) | 9.75 | 1 | 1 | 3 | 3 | 277 | 30.5 | 5.91 |
| | OS = Mangifera indica PE = 2 | | | | | | | | |
| | SV = 1 - [G5CBR3_MANIN] | | | | | | | | |
| A0A067EU20 | Citrate synthase OS = Citrus | 5.34 | 2 | 2 | 2 | 2 | 431 | 47.3 | 8.40 |
| | sinensis | | | | | | | | |
| | GN = CISIN_1g010304mg | | | | | | | | |
| | PE = 3 SV = 1 - | | | | | | | | |
| | [A0A067EU20_CITSI] | | | | | | | | |
| A0A067E473 | Citrate synthase OS = Citrus | 7.71 | 15 | 1 | 3 | 3 | 402 | 44.7 | 6.76 |
| | sinensis | | | | | | | | |
| | GN = CISIN_1g012107mg | | | | | | | | |
| | PE = 3 SV = 1 - | | | | | | | | |
| | [A0A067E473_CITSI] | | | | | | | | |
| V4V9V7 | Clathrin heavy chain | 4.88 | 2 | 8 | 8 | 8 | 1701 | 192.8 | 5.43 |
| | OS = Citrus clementina | | | | | | | | |
| | GN = CICLE_v10030488mg | | | | | | | | |
| | PE = 3 SV = 1 - | | | | | | | | |
| | [V4V9V7_9ROSI] | | | | | | | | |
| A0A067E2N7 | Coatomer subunit alpha | 2.57 | 4 | 3 | 3 | 3 | 1208 | 135.2 | 6.76 |
| | OS = Citrus sinensis | | | | | | | | |
| | GN = CISIN_1g000933mg | | | | | | | | |
| | PE = 4 SV = 1 - | | | | | | | | |
| | [A0A067E2N7_CITSI] | | | | | | | | |
| A0A067FV47 | Coatomer subunit beta | 2.19 | 3 | 2 | 2 | 2 | 867 | 97.3 | 5.88 |
| | OS = Citrus sinensis | | | | | | | | |
| | GN = CISIN_1g002235mg | | | | | | | | |
| | PE = 4 SV = 1 - | | | | | | | | |
| | [A0A067FV47_CITSI] | | | | | | | | |
| H2E0Q9 | Copper/zinc superoxide | 8.44 | 4 | 1 | 1 | 1 | 154 | 15.8 | 5.62 |
| | dismutase (Fragment) | | | | | | | | |
| | OS = Litchi chinensis PE = 2 | | | | | | | | |
| | SV = 1 - [H2E0Q9_LITCN] | | | | | | | | |
| H2DRJ4 | Copper/zinc superoxide | 8.00 | 7 | 1 | 1 | 1 | 125 | 12.5 | 6.09 |
| | dismutase (Fragment) | | | | | | | | |
| | OS = Tetradium ruticarpum | | | | | | | | |
| | PE = 2 SV = 1 - | | | | | | | | |
| | [H2DRJ4_9ROSI] | | | | | | | | |
| V4WBK6 | Cytochrome b-c1 complex | 9.02 | 1 | 1 | 1 | 1 | 122 | 14.5 | 9.36 |
| | subunit 7 OS = Citrus | | | | | | | | |
| | clementina | | | | | | | | |
| | GN = CICLE_v10009887mg | | | | | | | | |
| | PE = 3 SV = 1 - | | | | | | | | |
| | [V4WBK6_9ROSI] | | | | | | | | |
| A0A067FXK6 | Dihydrolipoyl | 9.07 | 3 | 4 | 4 | 5 | 507 | 53.8 | 7.40 |
| | dehydrogenase OS = Citrus | | | | | | | | |
| | sinensis | | | | | | | | |
| | GN = CISIN_1g010573mg | | | | | | | | |
| | PE = 4 SV = 1 - | | | | | | | | |
| | [A0A067FXK6_CITSI] | | | | | | | | |
| V4S7N0 | Dolichyl-diphosphooligosac | 4.98 | 3 | 2 | 2 | 2 | 422 | 47.1 | 5.66 |
| | charide--protein | | | | | | | | |
| | glycosyltransferase 48 kDa | | | | | | | | |
| | subunit OS = Citrus | | | | | | | | |
| | clementina | | | | | | | | |
| | GN = CICLE_v10028484mg | | | | | | | | |
| | PE = 3 SV = 1 - | | | | | | | | |
| | [V4S7N0_9ROSI] | | | | | | | | |
| D3TIC5 | Elongation factor 1-alpha | 55.81 | 1 | 1 | 1 | 2 | 43 | 4.8 | 8.22 |
| | (Fragment) OS = Mangifera | | | | | | | | |
| | indica PE = 2 SV = 1 - | | | | | | | | |
| | [D3TIC5_MANIN] | | | | | | | | |
| A0A067E3N7 | Elongation factor 1-alpha | 31.10 | 8 | 10 | 10 | 21 | 447 | 49.3 | 9.11 |
| | OS = Citrus sinensis | | | | | | | | |
| | GN = CISIN_1g0132372mg | | | | | | | | |
| | PE = 3 SV = 1 - | | | | | | | | |
| | [A0A067E3N7_CITSI] | | | | | | | | |
| V4UV38 | Enolase OS = Citrus | 32.58 | 4 | 4 | 8 | 20 | 356 | 38.2 | 5.90 |
| | clementina | | | | | | | | |
| | GN = CICLE_v10011726mg | | | | | | | | |
| | PE = 3 SV = 1 - | | | | | | | | |
| | [V4UV38_9ROSI] | | | | | | | | |
| E9NVZ3 | Enoyl-ACP reductase | 6.51 | 1 | 1 | 2 | 3 | 338 | 35.9 | 9.26 |
| | (Fragment) OS = Dimocarpus | | | | | | | | |
| | longan PE = 2 SV = 1 - | | | | | | | | |
| | [E9NVZ3_9ROSI] | | | | | | | | |
| A0A0H4LVI2 | Eukaryotic translation | 6.90 | 2 | 1 | 1 | 1 | 145 | 16.4 | 4.98 |
| | initiation factor 1Ab | | | | | | | | |
| | OS = Mangifera indica | | | | | | | | |
| | GN = EIF1Ab PE = 2 SV = 1 - | | | | | | | | |
| | [A0A0H4LVI2_MANIN] | | | | | | | | |
| A0A067H239 | Eukaryotic translation | 2.09 | 3 | 1 | 1 | 1 | 764 | 89.7 | 9.01 |
| | initiation factor 3 subunit A | | | | | | | | |
| | OS = Citrus sinensis | | | | | | | | |
| | GN = CISIN_1g003396mg | | | | | | | | |
| | PE = 3 SV = 1 - | | | | | | | | |
| | [A0A067H239_CITSI] | | | | | | | | |
| A0A067E325 | Eukaryotic translation | 1.59 | 2 | 1 | 1 | 1 | 439 | 51.7 | 6.30 |
| | initiation factor 3 subunit E | | | | | | | | |
| | OS = Citrus sinensis | | | | | | | | |
| | GN = CISIN_1g013640mg | | | | | | | | |
| | PE = 3 SV = 1 - | | | | | | | | |
| | [A0A067E325_CITSI] | | | | | | | | |
| A0A067FGY4 | Eukaryotic translation | 3.41 | 2 | 1 | 1 | 1 | 293 | 32.3 | 8.91 |
| | initiation factor 3 subunit G | | | | | | | | |
| | OS = Citrus sinensis | | | | | | | | |
| | GN = CISIN_1g022742mg | | | | | | | | |
| | PE = 3 SV = 1 - | | | | | | | | |
| | [A0A067FGY4_CITSI] | | | | | | | | |
| V4UI34 | Eukaryotic translation | 4.18 | 1 | 2 | 2 | 2 | 526 | 61.7 | 6.07 |
| | initiation factor 3 subunit L | | | | | | | | |
| | OS = Citrus clementina | | | | | | | | |
| | GN = CICLE_v10007989mg | | | | | | | | |
| | PE = 3 SV = 1 - | | | | | | | | |
| | [V4UI34_9ROSI] | | | | | | | | |
| V4TKH8 | Eukaryotic translation | 22.48 | 9 | 2 | 2 | 4 | 129 | 14.2 | 6.40 |
| | initiation factor 5A | | | | | | | | |
| | OS = Citrus clementina | | | | | | | | |
| | GN = CICLE_v10022624mg | | | | | | | | |
| | PE = 3 SV = 1 - | | | | | | | | |
| | [V4TKH8_9ROSI] | | | | | | | | |
| A0A067FVY8 | Eukaryotic translation | 4.90 | 2 | 1 | 1 | 1 | 245 | 26.5 | 4.81 |
| | initiation factor 6 OS = Citrus | | | | | | | | |
| | sinensis GN = EIF6 PE = 3 | | | | | | | | |
| | SV = 1 - | | | | | | | | |
| | [A0A067FVY8_CITSI] | | | | | | | | |
| A0A067FZI0 | Ferritin (Fragment) | 11.25 | 3 | 1 | 1 | 1 | 80 | 9.2 | 6.52 |
| | OS = Citrus sinensis | | | | | | | | |
| | GN = CISIN_1g0217031mg | | | | | | | | |
| | PE = 3 SV = 1 - | | | | | | | | |
| | [A0A067FZI0_CITSI] | | | | | | | | |
| A0A067E486 | Fructose-bisphosphate | 7.01 | 6 | 1 | 2 | 2 | 314 | 33.7 | 7.24 |
| | aldolase OS = Citrus sinensis | | | | | | | | |
| | GN = CISIN_1g015888mg | | | | | | | | |
| | PE = 3 SV = 1 - | | | | | | | | |
| | [A0A067E486_CITSI] | | | | | | | | |
| A0A067GAY2 | Fructose-bisphosphate | 20.51 | 3 | 4 | 5 | 5 | 395 | 43.0 | 8.69 |
| | aldolase OS = Citrus sinensis | | | | | | | | |
| | GN = CISIN_1g016109mg | | | | | | | | |
| | PE = 3 SV = 1 - | | | | | | | | |
| | [A0A067GAY2_CITSI] | | | | | | | | |
| A0A067F4J3 | Fructose-bisphosphate | 10.61 | 3 | 4 | 4 | 4 | 358 | 38.6 | 7.65 |
| | aldolase OS = Citrus sinensis | | | | | | | | |
| | GN = CISIN_1g018326mg | | | | | | | | |
| | PE = 3 SV = 1 - | | | | | | | | |
| | [A0A067F4J3_CITSI] | | | | | | | | |
| A0A097PMU7 | Galactose mutarotase-like | 6.07 | 2 | 1 | 1 | 1 | 214 | 23.4 | 6.54 |
| | superfamily protein | | | | | | | | |
| | (Fragment) OS = Citrus | | | | | | | | |
| | clementina PE = 2 SV = 1 - | | | | | | | | |
| | [A0A097PMU7_9ROSI] | | | | | | | | |
| V4T9V5 | Glucose-1-phosphate | 1.90 | 1 | 1 | 1 | 1 | 526 | 57.9 | 7.83 |
| | adenylyltransferase | | | | | | | | |
| | OS = Citrus clementina | | | | | | | | |
| | GN = CICLE_v10000842mg | | | | | | | | |
| | PE = 3 SV = 1 - | | | | | | | | |
| | [V4T9V5_9ROSI] | | | | | | | | |
| A0A067DU27 | Glucose-1-phosphate | 22.12 | 16 | 9 | 9 | 9 | 520 | 57.0 | 7.47 |
| | adenylyltransferase | | | | | | | | |
| | OS = Citrus sinensis | | | | | | | | |
| | GN = CISIN_1g009971mg | | | | | | | | |
| | PE = 3 SV = 1 - | | | | | | | | |
| | [A0A067DU27_CITSI] | | | | | | | | |
| A0A067H083 | Glucose-6-phosphate | 8.24 | 3 | 4 | 4 | 4 | 619 | 67.8 | 5.43 |
| | isomerase OS = Citrus | | | | | | | | |
| | sinensis | | | | | | | | |
| | GN = CISIN_1g007074mg | | | | | | | | |
| | PE = 3 SV = 1 - | | | | | | | | |
| | [A0A067H083_CITSI] | | | | | | | | |
| A0A067GY43 | Glucose-6-phosphate | 2.72 | 4 | 1 | 1 | 1 | 441 | 48.3 | 6.80 |
| | isomerase OS = Citrus | | | | | | | | |
| | sinensis | | | | | | | | |
| | GN = CISIN_1g007374mg | | | | | | | | |
| | PE = 3 SV = 1 - | | | | | | | | |
| | [A0A067GY43_CITSI] | | | | | | | | |
| V4U3F0 | Glutamate dehydrogenase | 14.36 | 6 | 1 | 4 | 5 | 411 | 44.4 | 7.09 |
| | OS = Citrus clementina | | | | | | | | |
| | GN = CICLE_v10020428mg | | | | | | | | |
| | PE = 3 SV = 1 - | | | | | | | | |
| | [V4U3F0_9ROSI] | | | | | | | | |
| D2XZ12 | Glutathione peroxidase | 6.61 | 1 | 1 | 1 | 1 | 121 | 13.5 | 6.62 |
| | (Fragment) OS = Mangifera | | | | | | | | |
| | indica GN = POD PE = 2 | | | | | | | | |
| | SV = 1 - [D2XZ12_MANIN] | | | | | | | | |
| V4TMD7 | Glutathione peroxidase | 15.06 | 8 | 2 | 2 | 2 | 166 | 19.2 | 6.57 |
| | OS = Citrus clementina | | | | | | | | |
| | GN = CICLE_v10022130mg | | | | | | | | |
| | PE = 3 SV = 1 - | | | | | | | | |
| | [V4TMD7_9ROSI] | | | | | | | | |
| B6DZD3 | Glutathione S-transferase | 9.72 | 4 | 1 | 2 | 3 | 216 | 25.0 | 7.33 |
| | Tau2 OS = Citrus sinensis | | | | | | | | |
| | GN = GSTU2 PE = 2 SV = 1 - | | | | | | | | |
| | [B6DZD3_CITSI] | | | | | | | | |
| V4VKP2 | Glyceraldehyde-3-phosphate | 31.16 | 11 | 4 | 8 | 40 | 337 | 36.7 | 7.96 |
| | dehydrogenase OS = Citrus | | | | | | | | |
| | clementina | | | | | | | | |
| | GN = CICLE_v10021037mg | | | | | | | | |
| | PE = 3 SV = 1 - | | | | | | | | |
| | [V4VKP2_9ROSI] | | | | | | | | |
| V4VFS1 | Glyceraldehyde-3-phosphate | 34.60 | 15 | 2 | 9 | 48 | 341 | 37.2 | 7.93 |
| | dehydrogenase OS = Citrus | | | | | | | | |
| | clementina | | | | | | | | |
| | GN = CICLE_v10032014mg | | | | | | | | |
| | PE = 3 SV = 1 - | | | | | | | | |
| | [V4VFS1_9ROSI] | | | | | | | | |
| C0LDX1 | Glyceraldehyde-3-phosphate | 27.98 | 9 | 1 | 8 | 34 | 336 | 36.5 | 7.52 |
| | dehydrogenase | | | | | | | | |
| | OS = Dimocarpus longan | | | | | | | | |
| | PE = 2 SV = 1 - | | | | | | | | |
| | [C0LDX1_9ROSI] | | | | | | | | |
| A0A067H6T8 | Glycerol-3-phosphate | 5.64 | 6 | 2 | 2 | 2 | 461 | 51.7 | 6.70 |
| | dehydrogenase [NAD(+)] | | | | | | | | |
| | OS = Citrus sinensis | | | | | | | | |
| | GN = CISIN_1g012349mg | | | | | | | | |
| | PE = 3 SV = 1 - | | | | | | | | |
| | [A0A067H6T8_CITSI] | | | | | | | | |
| Q9MBF3 | Glycine-rich RNA-binding | 10.78 | 1 | 2 | 2 | 3 | 167 | 16.8 | 8.13 |
| | protein OS = Citrus unshiu | | | | | | | | |
| | PE = 4 SV = 1 - | | | | | | | | |
| | [Q9MBF3_CITUN] | | | | | | | | |
| D2J2L0 | Granule-bound starch | 23.70 | 2 | 3 | 3 | 3 | 135 | 15.0 | 5.96 |
| | synthase I (Fragment) | | | | | | | | |
| | OS = Bursera graveolens | | | | | | | | |
| | GN = GBSSI PE = 4 SV = 1 - | | | | | | | | |
| | [D2J2L0_9ROSI] | | | | | | | | |
| A0A067E6X0 | GrpE protein homolog | 4.07 | 5 | 1 | 1 | 1 | 221 | 24.7 | 4.65 |
| | OS = Citrus sinensis | | | | | | | | |
| | GN = CISIN_1g019043mg | | | | | | | | |
| | PE = 3 SV = 1 - | | | | | | | | |
| | [A0A067E6X0_CITSI] | | | | | | | | |
| F8SVK6 | H+-pyrophosphatase | 4.91 | 1 | 1 | 1 | 1 | 163 | 16.8 | 8.73 |
| | (Fragment) OS = Dimocarpus | | | | | | | | |
| | longan PE = 2 SV = 1 - | | | | | | | | |
| | [F8SVK6_9ROSI] | | | | | | | | |
| A0A0H3UZ51 | Heat-shock protein 17.6 | 50.00 | 2 | 4 | 7 | 32 | 154 | 17.5 | 6.06 |
| | OS = Mangifera indica | | | | | | | | |
| | GN = Hsp17.6 PE = 2 SV = 1 - | | | | | | | | |
| | [A0A0H3UZ51_MANIN] | | | | | | | | |
| V4UH09 | Histone H2A OS = Citrus | 11.94 | 7 | 1 | 2 | 3 | 134 | 14.0 | 10.05 |
| | clementina | | | | | | | | |
| | GN = CICLE_v10009865mg | | | | | | | | |
| | PE = 3 SV = 1 - | | | | | | | | |
| | [V4UH09_9ROSI] | | | | | | | | |
| V4RZA6 | Histone H2A OS = Citrus | 11.85 | 8 | 1 | 2 | 3 | 135 | 14.4 | 10.29 |
| | clementina | | | | | | | | |
| | GN = CICLE_v10026746mg | | | | | | | | |
| | PE = 3 SV = 1 - | | | | | | | | |
| | [V4RZA6_9ROSI] | | | | | | | | |
| A0A067EQW1 | Histone H2A OS = Citrus | 11.19 | 7 | 1 | 2 | 3 | 143 | 15.3 | 10.67 |
| | sinensis | | | | | | | | |
| | GN = CISIN_1g032326mg | | | | | | | | |
| | PE = 3 SV = 1 - | | | | | | | | |
| | [A0A067EQW1_CITSI] | | | | | | | | |
| A0A068PSV8 | Histone H3 (Fragment) | 6.98 | 4 | 1 | 1 | 2 | 129 | 14.4 | 11.18 |
| | OS = Zanthoxylum | | | | | | | | |
| | ailanthoides GN = CENH3 | | | | | | | | |
| | PE = 3 SV = 1 - | | | | | | | | |
| | [A0A068PSV8_9ROSI] | | | | | | | | |
| Q948T8 | Histone H4 (Fragment) | 29.41 | 3 | 3 | 3 | 3 | 102 | 11.3 | 11.47 |
| | OS = Citrus jambhiri | | | | | | | | |
| | GN = histone H4 PE = 2 SV = 1 - | | | | | | | | |
| | [Q948T8_CITJA] | | | | | | | | |
| Q84LP3 | HSP22 (Fragment) | 23.33 | 14 | 1 | 2 | 2 | 60 | 7.2 | 8.56 |
| | OS = Citrus paradisi PE = 2 | | | | | | | | |
| | SV = 1 - [Q84LP3_CITPA] | | | | | | | | |
| H9NHJ9 | Hsp90 OS = Citrus sinensis | 11.29 | 2 | 5 | 7 | 9 | 700 | 80.0 | 5.11 |
| | GN = hsp90 PE = 2 SV = 1 - | | | | | | | | |
| | [H9NHJ9_CITSI] | | | | | | | | |
| V4T2Q5 | Isocitrate dehydrogenase | 25.85 | 21 | 10 | 10 | 12 | 414 | 46.5 | 7.18 |
| | [NADP] OS = Citrus | | | | | | | | |
| | clementina | | | | | | | | |
| | GN = CICLE_v10001314mg | | | | | | | | |
| | PE = 3 SV = 1 - | | | | | | | | |
| | [V4T2Q5_9ROSI] | | | | | | | | |
| A0A067EXL9 | Isocitrate lyase OS = Citrus | 4.87 | 2 | 2 | 2 | 4 | 575 | 64.3 | 7.34 |
| | sinensis | | | | | | | | |
| | GN = CISIN_1g042063mg | | | | | | | | |
| | PE = 3 SV = 1 - | | | | | | | | |
| | [A0A067EXL9_CITSI] | | | | | | | | |
| A0A067GJJ8 | Lactoylglutathione lyase | 8.42 | 12 | 3 | 3 | 3 | 285 | 31.9 | 5.63 |
| | OS = Citrus sinensis | | | | | | | | |
| | GN = CISIN_1g016689mg | | | | | | | | |
| | PE = 3 SV = 1 - | | | | | | | | |
| | [A0A067GJJ8_CITSI] | | | | | | | | |
| A0A067HCN8 | Malate dehydrogenase | 14.24 | 5 | 1 | 3 | 4 | 309 | 33.5 | 6.87 |
| | OS = Citrus sinensis | | | | | | | | |
| | GN = CISIN_1g017740mg | | | | | | | | |
| | PE = 3 SV = 1 - | | | | | | | | |
| | [A0A067HCN8_CITSI] | | | | | | | | |
| A0A067GGN6 | Malate dehydrogenase | 18.85 | 19 | 2 | 3 | 3 | 244 | 25.8 | 7.97 |
| | OS = Citrus sinensis | | | | | | | | |
| | GN = CISIN_1g018265mg | | | | | | | | |
| | PE = 3 SV = 1 - | | | | | | | | |
| | [A0A067GGN6_CITSI] | | | | | | | | |
| A0A067EBQ5 | Malate dehydrogenase | 21.09 | 10 | 2 | 4 | 4 | 256 | 26.6 | 9.22 |
| | OS = Citrus sinensis | | | | | | | | |
| | GN = CISIN_1g018314mg | | | | | | | | |
| | PE = 3 SV = 1 - | | | | | | | | |
| | [A0A067EBQ5_CITSI] | | | | | | | | |
| A0A067F301 | Malate dehydrogenase | 14.67 | 6 | 1 | 3 | 4 | 300 | 32.0 | 7.21 |
| | OS = Citrus sinensis | | | | | | | | |
| | GN = CISIN_1g020022mg | | | | | | | | |
| | PE = 3 SV = 1 - | | | | | | | | |
| | [A0A067F301_CITSI] | | | | | | | | |
| V4TNQ8 | Malate synthase OS = Citrus | 4.57 | 1 | 3 | 3 | 3 | 569 | 64.5 | 7.85 |
| | clementina | | | | | | | | |
| | GN = CICLE_v10019499mg | | | | | | | | |
| | PE = 3 SV = 1 - | | | | | | | | |
| | [V4TNQ8_9ROSI] | | | | | | | | |
| A0A067E9C1 | Malic enzyme OS = Citrus | 1.81 | 4 | 1 | 1 | 1 | 497 | 55.0 | 6.14 |
| | sinensis | | | | | | | | |
| | GN = CISIN_1g006454mg | | | | | | | | |
| | PE = 3 SV = 1 - | | | | | | | | |
| | [A0A067E9C1_CITSI] | | | | | | | | |
| A0A067DVP8 | Methionine aminopeptidase | 2.35 | 2 | 1 | 1 | 1 | 425 | 47.9 | 5.92 |
| | 2 OS = Citrus sinensis | | | | | | | | |
| | GN = CISIN_1g014386mg | | | | | | | | |
| | PE = 3 SV = 1 - | | | | | | | | |
| | [A0A067DVP8_CITSI] | | | | | | | | |
| A0A067G2L2 | Methylthioribose-1-phosphate | 7.26 | 5 | 2 | 2 | 2 | 358 | 37.7 | 5.72 |
| | isomerase OS = Citrus | | | | | | | | |
| | sinensis | | | | | | | | |
| | GN = CISIN_1g017293mg | | | | | | | | |
| | PE = 3 SV = 1 - | | | | | | | | |
| | [A0A067G2L2_CITSI] | | | | | | | | |
| A0A067DLL8 | N-acetyl-gamma-glutamyl-phosphate | 2.96 | 2 | 1 | 1 | 1 | 406 | 44.5 | 8.37 |
| | reductase | | | | | | | | |
| | OS = Citrus sinensis | | | | | | | | |
| | GN = CISIN_1g015441mg | | | | | | | | |
| | PE = 3 SV = 1 - | | | | | | | | |
| | [A0A067DLL8_CITSI] | | | | | | | | |
| G8HI95 | NAD-dependent isocitrate | 15.33 | 5 | 1 | 1 | 1 | 150 | 16.5 | 6.16 |
| | dehydrogenase (Fragment) | | | | | | | | |
| | OS = Citrus maxima PE = 2 | | | | | | | | |
| | SV = 1 - [G8HI95_CITMA] | | | | | | | | |
| A0A067EX46 | Nascent | 23.87 | 2 | 2 | 2 | 3 | 155 | 16.9 | 5.21 |
| | polypeptide-associated | | | | | | | | |
| | complex subunit beta | | | | | | | | |
| | OS = Citrus sinensis | | | | | | | | |
| | GN = CISIN_1g031135mg | | | | | | | | |
| | PE = 3 SV = 1 - | | | | | | | | |
| | [A0A067EX46_CITSI] | | | | | | | | |
| F5A8C7 | Non-specific lipid-transfer | 6.96 | 2 | 1 | 1 | 1 | 115 | 11.7 | 8.95 |
| | protein OS = Dimocarpus | | | | | | | | |
| | longan GN = LTP PE = 2 SV = 1 - | | | | | | | | |
| | [F5A8C7_9ROSI] | | | | | | | | |
| A0A067FT42 | Nucleoside diphosphate | 12.12 | 3 | 1 | 1 | 1 | 132 | 14.6 | 7.24 |
| | kinase (Fragment) | | | | | | | | |
| | OS = Citrus sinensis | | | | | | | | |
| | GN = CISIN_1g045706mg | | | | | | | | |
| | PE = 3 SV = 1 - | | | | | | | | |
| | [A0A067FT42_CITSI] | | | | | | | | |
| V4RIV9 | Orotidine 5′-phosphate | 7.94 | 2 | 3 | 3 | 3 | 340 | 37.0 | 6.71 |
| | decarboxylase (Fragment) | | | | | | | | |
| | OS = Citrus clementina | | | | | | | | |
| | GN = CICLE_v100049161mg | | | | | | | | |
| | PE = 3 SV = 1 - | | | | | | | | |
| | [V4RIV9_9ROSI] | | | | | | | | |
| A0A067H1L1 | Peptidyl-prolyl cis-trans | 4.60 | 5 | 1 | 1 | 1 | 174 | 19.8 | 5.36 |
| | isomerase (Fragment) | | | | | | | | |
| | OS = Citrus sinensis | | | | | | | | |
| | GN = CISIN_1g0137551mg | | | | | | | | |
| | PE = 4 SV = 1 - | | | | | | | | |
| | [A0A067H1L1_CITSI] | | | | | | | | |
| A0A068PT04 | Peptidyl-prolyl cis-trans | 28.77 | 1 | 1 | 3 | 5 | 146 | 15.2 | 8.46 |
| | isomerase (Fragment) | | | | | | | | |
| | OS = Zanthoxylum | | | | | | | | |
| | ailanthoides GN = ROC1 | | | | | | | | |
| | PE = 3 SV = 1 - | | | | | | | | |
| | [A0A068PT04_9ROSI] | | | | | | | | |
| A0A067DBT5 | Peptidyl-prolyl cis-trans | 2.44 | 9 | 1 | 1 | 1 | 492 | 54.6 | 5.33 |
| | isomerase OS = Citrus | | | | | | | | |
| | sinensis | | | | | | | | |
| | GN = CISIN_1g008113mg | | | | | | | | |
| | PE = 4 SV = 1 - | | | | | | | | |
| | [A0A067DBT5_CITSI] | | | | | | | | |
| A0A067GQ09 | Peptidyl-prolyl cis-trans | 10.31 | 2 | 2 | 2 | 2 | 262 | 28.0 | 9.17 |
| | isomerase OS = Citrus | | | | | | | | |
| | sinensis | | | | | | | | |
| | GN = CISIN_1g024818mg | | | | | | | | |
| | PE = 3 SV = 1 - | | | | | | | | |
| | [A0A067GQ09_CITSI] | | | | | | | | |
| A0A067FS21 | Peptidyl-prolyl cis-trans | 4.04 | 8 | 1 | 1 | 1 | 198 | 21.3 | 9.13 |
| | isomerase OS = Citrus | | | | | | | | |
| | sinensis | | | | | | | | |
| | GN = CISIN_1g027222mg | | | | | | | | |
| | PE = 3 SV = 1 - | | | | | | | | |
| | [A0A067FS21_CITSI] | | | | | | | | |
| A0A067DWK9 | Peptidyl-prolyl cis-trans | 11.36 | 4 | 1 | 1 | 1 | 88 | 9.6 | 9.64 |
| | isomerase OS = Citrus | | | | | | | | |
| | sinensis | | | | | | | | |
| | GN = CISIN_1g033778mg | | | | | | | | |
| | PE = 4 SV = 1 - | | | | | | | | |
| | [A0A067DWK9_CITSI] | | | | | | | | |
| A0A0B6VMU5 | Peptidyl-prolyl cis-trans | 25.58 | 4 | 2 | 4 | 20 | 172 | 18.1 | 8.46 |
| | isomerase OS = Citrus | | | | | | | | |
| | tamurana GN = CCP PE = 2 | | | | | | | | |
| | SV = 1 - | | | | | | | | |
| | [A0A0B6VMU5_9ROSI] | | | | | | | | |
| D5H3K4 | Phosphoenol pyruvate | 36.47 | 5 | 1 | 3 | 4 | 85 | 9.9 | 7.25 |
| | carboxykinase (Fragment) | | | | | | | | |
| | OS = Santiria trimera | | | | | | | | |
| | GN = pepC PE = 4 SV = 1 - | | | | | | | | |
| | [D5H3K4_9ROSI] | | | | | | | | |
| H6BG02 | Phosphoenolpyruvate | 14.89 | 1 | 1 | 1 | 1 | 47 | 5.4 | 8.21 |
| | carboxylase (Fragment) | | | | | | | | |
| | OS = Bursera ovalifolia | | | | | | | | |
| | GN = pepC PE = 4 SV = 1 - | | | | | | | | |
| | [H6BG02_9ROSI] | | | | | | | | |
| A0A067F2H8 | Phosphoglycerate kinase | 11.39 | 3 | 3 | 4 | 7 | 474 | 49.4 | 8.15 |
| | OS = Citrus sinensis | | | | | | | | |
| | GN = CISIN_1g011942mg | | | | | | | | |
| | PE = 3 SV = 1 - | | | | | | | | |
| | [A0A067F2H8_CITSI] | | | | | | | | |
| A0A067FDS3 | Phosphoglycerate kinase | 21.45 | 2 | 6 | 7 | 8 | 401 | 42.3 | 6.29 |
| | OS = Citrus sinensis | | | | | | | | |
| | GN = CISIN_1g040813mg | | | | | | | | |
| | PE = 3 SV = 1 - | | | | | | | | |
| | [A0A067FDS3_CITSI] | | | | | | | | |
| A0A067FVR0 | Phosphoserine | 2.09 | 2 | 1 | 1 | 1 | 430 | 47.7 | 8.29 |
| | aminotransferase OS = Citrus | | | | | | | | |
| | sinensis | | | | | | | | |
| | GN = CISIN_1g043610mg | | | | | | | | |
| | PE = 3 SV = 1 - | | | | | | | | |
| | [A0A067FVR0_CITSI] | | | | | | | | |
| B7P073 | Pis v 2.0101 allergen11S | 11.69 | 1 | 4 | 5 | 26 | 496 | 56.4 | 7.55 |
| | globulin precusor | | | | | | | | |
| | OS = Pistacia vera PE = 2 | | | | | | | | |
| | SV = 1 - [B7P073_PISVE] | | | | | | | | |
| B7SLJ1 | Pis v 5.0101 allergen 11S | 7.61 | 1 | 1 | 2 | 22 | 473 | 53.3 | 6.23 |
| | globulin precusor | | | | | | | | |
| | OS = Pistacia vera PE = 2 | | | | | | | | |
| | SV = 1 - [B7SLJ1_PISVE] | | | | | | | | |
| A0A067EVU5 | Poly [ADP-ribose] | 3.44 | 2 | 2 | 2 | 2 | 815 | 91.1 | 5.59 |
| | polymerase OS = Citrus | | | | | | | | |
| | sinensis | | | | | | | | |
| | GN = CISIN_1g037230mg | | | | | | | | |
| | PE = 4 SV = 1 - | | | | | | | | |
| | [A0A067EVU5_CITSI] | | | | | | | | |
| A0A067FXJ2 | Proline iminopeptidase | 2.95 | 4 | 1 | 1 | 1 | 237 | 27.0 | 5.15 |
| | OS = Citrus sinensis | | | | | | | | |
| | GN = CISIN_1g020064mg | | | | | | | | |
| | PE = 3 SV = 1 - | | | | | | | | |
| | [A0A067FXJ2_CITSI] | | | | | | | | |
| V4SS21 | Proteasome subunit alpha | 29.76 | 10 | 2 | 2 | 2 | 84 | 9.0 | 9.64 |
| | type (Fragment) OS = Citrus | | | | | | | | |
| | clementina | | | | | | | | |
| | GN = CICLE_v100263521mg | | | | | | | | |
| | PE = 3 SV = 1 - | | | | | | | | |
| | [V4SS21_9ROSI] | | | | | | | | |
| V4T9U5 | Proteasome subunit alpha | 12.73 | 3 | 3 | 3 | 3 | 275 | 29.7 | 5.27 |
| | type OS = Citrus clementina | | | | | | | | |
| | GN = CICLE_v10002110mg | | | | | | | | |
| | PE = 3 SV = 1 - | | | | | | | | |
| | [V4T9U5_9ROSI] | | | | | | | | |
| V4RMI5 | Proteasome subunit alpha | 8.59 | 3 | 1 | 1 | 1 | 198 | 21.6 | 8.28 |
| | type OS = Citrus clementina | | | | | | | | |
| | GN = CICLE_v10005774mg | | | | | | | | |
| | PE = 3 SV = 1 - | | | | | | | | |
| | [V4RMI5_9ROSI] | | | | | | | | |
| V4SSB8 | Proteasome subunit alpha | 22.78 | 3 | 4 | 4 | 4 | 237 | 26.0 | 4.70 |
| | type OS = Citrus clementina | | | | | | | | |
| | GN = CICLE_v10026377mg | | | | | | | | |
| | PE = 3 SV = 1 - | | | | | | | | |
| | [V4SSB8_9ROSI] | | | | | | | | |
| A0A067EH28 | Proteasome subunit alpha | 3.60 | 2 | 1 | 1 | 1 | 250 | 27.3 | 7.23 |
| | type OS = Citrus sinensis | | | | | | | | |
| | GN = CISIN_1g025640mg | | | | | | | | |
| | PE = 3 SV = 1 - | | | | | | | | |
| | [A0A067EH28_CITSI] | | | | | | | | |
| A0A067G6V9 | Proteasome subunit alpha | 10.04 | 6 | 2 | 2 | 3 | 249 | 27.2 | 8.84 |
| | type OS = Citrus sinensis | | | | | | | | |
| | GN = CISIN_1g025716mg | | | | | | | | |
| | PE = 3 SV = 1 - | | | | | | | | |
| | [A0A067G6V9_CITSI] | | | | | | | | |
| C3VIB9 | Proteasome subunit beta | 9.79 | 3 | 1 | 1 | 1 | 194 | 21.0 | 7.49 |
| | type (Fragment) OS = Citrus | | | | | | | | |
| | maxima PE = 2 SV = 1 - | | | | | | | | |
| | [C3VIB9_CITMA] | | | | | | | | |
| V4TS21 | Proteasome subunit beta | 11.44 | 3 | 3 | 3 | 3 | 236 | 25.6 | 5.38 |
| | type OS = Citrus clementina | | | | | | | | |
| | GN = CICLE_v10009349mg | | | | | | | | |
| | PE = 3 SV = 1 - | | | | | | | | |
| | [V4TS21_9ROSI] | | | | | | | | |
| A0A067G0K3 | Proteasome subunit beta | 5.79 | 2 | 1 | 1 | 1 | 242 | 27.1 | 7.53 |
| | type OS = Citrus sinensis | | | | | | | | |
| | GN = CISIN_1g026175mg | | | | | | | | |
| | PE = 3 SV = 1 - | | | | | | | | |
| | [A0A067G0K3_CITSI] | | | | | | | | |
| A0A067GYQ8 | Protein disulfide-isomerase | 6.12 | 3 | 2 | 2 | 2 | 441 | 49.1 | 4.94 |
| | (Fragment) OS = Citrus | | | | | | | | |
| | sinensis | | | | | | | | |
| | GN = CISIN_1g0108041mg | | | | | | | | |
| | PE = 3 SV = 1 - | | | | | | | | |
| | [A0A067GYQ8_CITSI] | | | | | | | | |
| A0A067G1S0 | Protein-L-isoaspartate | 9.30 | 5 | 2 | 2 | 2 | 215 | 23.1 | 5.92 |
| | O-methyltransferase | | | | | | | | |
| | OS = Citrus sinensis | | | | | | | | |
| | GN = CISIN_1g022070mg | | | | | | | | |
| | PE = 3 SV = 1 - | | | | | | | | |
| | [A0A067G1S0_CITSI] | | | | | | | | |
| B7UCK1 | Putative aconitase | 8.83 | 1 | 1 | 2 | 2 | 317 | 34.0 | 6.35 |
| | (Fragment) OS = Dimocarpus | | | | | | | | |
| | longan PE = 2 SV = 1 - | | | | | | | | |
| | [B7UCK1_9ROSI] | | | | | | | | |
| D0VZW1 | Putative ADP-glucose | 13.54 | 4 | 1 | 1 | 1 | 96 | 10.9 | 5.39 |
| | pyrophosphorylase large | | | | | | | | |
| | subunit (Fragment) | | | | | | | | |
| | OS = Zanthoxylum | | | | | | | | |
| | ailanthoides GN = agpL PE = 4 | | | | | | | | |
| | SV = 1 - [D0VZW1_9ROSI] | | | | | | | | |
| B2DEJ3 | Putative ADP-glucose | 7.08 | 11 | 1 | 1 | 1 | 113 | 12.6 | 7.21 |
| | pyrophosphorylase large | | | | | | | | |
| | subunit (Fragment) | | | | | | | | |
| | OS = Zanthoxylum | | | | | | | | |
| | schinifolium GN = agpL | | | | | | | | |
| | PE = 4 SV = 1 - | | | | | | | | |
| | [B2DEJ3_9ROSI] | | | | | | | | |
| B2DDZ3 | Putative granule-bound | 12.83 | 6 | 1 | 2 | 5 | 187 | 21.4 | 8.41 |
| | starch synthase (Fragment) | | | | | | | | |
| | OS = Zanthoxylum | | | | | | | | |
| | ailanthoides GN = GBSSI | | | | | | | | |
| | PE = 4 SV = 1 - | | | | | | | | |
| | [B2DDZ3_9ROSI] | | | | | | | | |
| Q94FU6 | Putative mitochondrial | 7.02 | 4 | 2 | 2 | 2 | 242 | 25.7 | 9.69 |
| | uncoupling protein | | | | | | | | |
| | (Fragment) OS = Mangifera | | | | | | | | |
| | indica PE = 3 SV = 1 - | | | | | | | | |
| | [Q94FU6_MANIN] | | | | | | | | |
| D3GGJ6 | Putative plasma membrane | 7.74 | 1 | 1 | 1 | 1 | 155 | 16.3 | 8.47 |
| | intrinsic protein PIP family | | | | | | | | |
| | member 1 aquaporin | | | | | | | | |
| | (Fragment) OS = Cedrela | | | | | | | | |
| | odorata PE = 3 SV = 1 - | | | | | | | | |
| | [D3GGJ6_9ROSI] | | | | | | | | |
| C9E0S6 | Putative uncharacterized | 3.69 | 1 | 1 | 1 | 1 | 244 | 28.6 | 9.95 |
| | protein OS = Dimocarpus | | | | | | | | |
| | longan PE = 2 SV = 2 - | | | | | | | | |
| | [C9E0S6_9ROSI] | | | | | | | | |
| A9YVC9 | Pyrophosphate-dependent | 7.61 | 11 | 4 | 4 | 4 | 565 | 61.5 | 6.74 |
| | phosphofructokinase beta | | | | | | | | |
| | subunit OS = Citrus sinensis x | | | | | | | | |
| | Citrus trifoliata PE = 2 SV = 1 - | | | | | | | | |
| | [A9YVC9_9ROSI] | | | | | | | | |
| Q4JIY7 | Pyruvate decarboxylase | 8.54 | 7 | 1 | 1 | 1 | 199 | 22.1 | 5.40 |
| | (Fragment) OS = Citrus | | | | | | | | |
| | sinensis PE = 2 SV = 1 - | | | | | | | | |
| | [Q4JIY7_CITSI] | | | | | | | | |
| A0A067HF55 | Pyruvate dehydrogenase E1 | 3.54 | 5 | 1 | 1 | 1 | 339 | 37.5 | 6.39 |
| | component subunit alpha | | | | | | | | |
| | OS = Citrus sinensis | | | | | | | | |
| | GN = CISIN_1g013928mg | | | | | | | | |
| | PE = 4 SV = 1 - | | | | | | | | |
| | [A0A067HF55_CITSI] | | | | | | | | |
| V4S7W4 | Pyruvate kinase OS = Citrus | 26.94 | 3 | 11 | 11 | 14 | 527 | 57.2 | 7.85 |
| | clementina | | | | | | | | |
| | GN = CICLE_v10011472mg | | | | | | | | |
| | PE = 3 SV = 1 - | | | | | | | | |
| | [V4S7W4_9ROSI] | | | | | | | | |
| V4TYW6 | Pyruvate kinase OS = Citrus | 6.53 | 3 | 2 | 2 | 2 | 505 | 54.5 | 8.13 |
| | clementina | | | | | | | | |
| | GN = CICLE_v10014977mg | | | | | | | | |
| | PE = 3 SV = 1 - | | | | | | | | |
| | [V4TYW6_9ROSI] | | | | | | | | |
| A0A067GEG0 | Pyruvate kinase OS = Citrus | 14.95 | 7 | 5 | 5 | 6 | 388 | 41.9 | 8.15 |
| | sinensis | | | | | | | | |
| | GN = CISIN_1g010443mg | | | | | | | | |
| | PE = 3 SV = 1 - | | | | | | | | |
| | [A0A067GEG0_CITSI] | | | | | | | | |
| Q64GC5 | Receptor-type protein kinase | 4.49 | 53 | 1 | 1 | 1 | 178 | 20.2 | 8.56 |
| | LRK (Fragment) | | | | | | | | |
| | OS = Mangifera indica PE = 4 | | | | | | | | |
| | SV = 1 - [Q64GC5_MANIN] | | | | | | | | |
| A0A067G8D2 | Ribosomal protein L19 | 9.19 | 8 | 1 | 1 | 1 | 185 | 22.1 | 11.50 |
| | OS = Citrus sinensis | | | | | | | | |
| | GN = CISIN_1g028783mg | | | | | | | | |
| | PE = 3 SV = 1 - | | | | | | | | |
| | [A0A067G8D2_CITSI] | | | | | | | | |
| G0ZDY0 | Ribosomal protein L27a | 10.20 | 1 | 1 | 1 | 1 | 98 | 11.0 | 9.91 |
| | (Fragment) OS = Dimocarpus | | | | | | | | |
| | longan GN = L27a PE = 2 | | | | | | | | |
| | SV = 1 - [G0ZDY0_9ROSI] | | | | | | | | |
| A0A067F4E3 | Ribosomal protein | 20.28 | 6 | 4 | 4 | 5 | 217 | 24.6 | 9.83 |
| | OS = Citrus sinensis | | | | | | | | |
| | GN = CISIN_1g027063mg | | | | | | | | |
| | PE = 3 SV = 1 - | | | | | | | | |
| | [A0A067F4E3_CITSI] | | | | | | | | |
| O98552 | Ribulose 1,5-bisphosphate | 29.31 | 702 | 1 | 11 | 24 | 447 | 49.4 | 6.37 |
| | carboxylase (Fragment) | | | | | | | | |
| | OS = Aegle marmelos | | | | | | | | |
| | GN = rbcL PE = 3 SV = 1 - | | | | | | | | |
| | [O98552_AEGMA] | | | | | | | | |
| Q646J1 | Ribulose 1,5-bisphosphate | 30.11 | 738 | 1 | 12 | 32 | 475 | 52.6 | 6.68 |
| | carboxylase large subunit | | | | | | | | |
| | OS = Lepisanthes alata | | | | | | | | |
| | GN = rbcL PE = 3 SV = 1 - | | | | | | | | |
| | [Q646J1_9ROSI] | | | | | | | | |
| D6C5Z5 | Ribulose bisphosphate | 28.01 | 738 | 1 | 11 | 29 | 457 | 50.6 | 5.36 |
| | carboxylase large chain | | | | | | | | |
| | (Fragment) OS = Canarium | | | | | | | | |
| | acutifolium GN = rbcL PE = 3 | | | | | | | | |
| | SV = 1 - [D6C5Z5_9ROSI] | | | | | | | | |
| J7Q5Q6 | Ribulose bisphosphate | 42.49 | 688 | 1 | 6 | 9 | 193 | 21.6 | 6.20 |
| | carboxylase large chain | | | | | | | | |
| | (Fragment) OS = Cotinus | | | | | | | | |
| | coggygria GN = rbcL PE = 3 | | | | | | | | |
| | SV = 1 - [J7Q5Q6_COTCG] | | | | | | | | |
| A0A0A7BXS8 | Ribulose bisphosphate | 35.29 | 92 | 1 | 3 | 7 | 170 | 18.8 | 6.14 |
| | calboxylase large chain | | | | | | | | |
| | (Fragment) | | | | | | | | |
| | OS = Zanthoxylum punctatum | | | | | | | | |
| | GN = rbcL PE = 3 SV = 1 - | | | | | | | | |
| | [A0A0A7BXS8_9ROSI] | | | | | | | | |
| A0A067DXX0 | Ribulose bisphosphate | 9.30 | 4 | 2 | 2 | 3 | 172 | 19.4 | 8.88 |
| | carboxylase small chain | | | | | | | | |
| | OS = Citrus sinensis | | | | | | | | |
| | GN = CISIN_1g029245mg | | | | | | | | |
| | PE = 3 SV = 1 - | | | | | | | | |
| | [A0A067DXX0_CITSI] | | | | | | | | |
| C6G3A0 | Ribulose-1,5-bisphosphate | 36.20 | 1059 | 1 | 14 | 43 | 453 | 50.3 | 6.33 |
| | carboxylase/oxygenase large | | | | | | | | |
| | subunit (Fragment) | | | | | | | | |
| | OS = Canarium vulgare | | | | | | | | |
| | GN = rbcL PE = 3 SV = 1 - | | | | | | | | |
| | [C6G3A0_9ROSI] | | | | | | | | |
| D9U8J5 | Ribulose-1,5-bisphosphate | 36.06 | 695 | 1 | 7 | 10 | 269 | 30.0 | 6.40 |
| | carboxylase/oxygenase large | | | | | | | | |
| | subunit (Fragment) | | | | | | | | |
| | OS = Cneoridium dumosum | | | | | | | | |
| | GN = rbcL PE = 3 SV = 1 - | | | | | | | | |
| | [D9U8J5_9ROSI] | | | | | | | | |
| W8CFF2 | Ribulose-1,5-bisphosphate | 37.69 | 1146 | 1 | 14 | 37 | 451 | 49.9 | 6.64 |
| | carboxylase/oxygenase large | | | | | | | | |
| | subunit (Fragment) | | | | | | | | |
| | OS = Comocladia engleriana | | | | | | | | |
| | GN = rbcL PE = 3 SV = 1 - | | | | | | | | |
| | [W8CFF2_9ROSI] | | | | | | | | |
| A0A0K2SQY8 | Ribulose-1,5-bisphosphate | 36.11 | 69 | 2 | 12 | 28 | 360 | 40.0 | 7.58 |
| | carboxylase/oxygenase large | | | | | | | | |
| | subunit OS = Melia volkensii | | | | | | | | |
| | GN = rbcL PE = 4 SV = 1 - | | | | | | | | |
| | [A0A0K2SQY8_9ROSI] | | | | | | | | |
| V4U4X3 | Serine | 17.20 | 4 | 6 | 6 | 6 | 471 | 51.8 | 7.11 |
| | hydroxymethyltransferase | | | | | | | | |
| | OS = Citrus clementina | | | | | | | | |
| | GN = CICLE_v10004902mg | | | | | | | | |
| | PE = 3 SV = 1 - | | | | | | | | |
| | [V4U4X3_9ROSI] | | | | | | | | |
| W0I5X0 | Small GTP Rab-GDI | 15.09 | 1 | 2 | 6 | 6 | 444 | 49.7 | 5.76 |
| | OS = Mangifera indica | | | | | | | | |
| | GN = rab-GDI PE = 2 SV = 1 - | | | | | | | | |
| | [W0I5X0_MANIN] | | | | | | | | |
| D3TIC0 | Small heat shock protein | 8.05 | 1 | 1 | 1 | 2 | 174 | 19.9 | 5.39 |
| | (Fragment) OS = Mangifera | | | | | | | | |
| | indica PE = 2 SV = 1 - | | | | | | | | |
| | [D3TIC0_MANIN] | | | | | | | | |
| V4UF13 | Small ubiquitin-related | 15.46 | 4 | 1 | 1 | 1 | 97 | 11.1 | 4.94 |
| | modifier OS = Citrus | | | | | | | | |
| | clementina | | | | | | | | |
| | GN = CICLE_v10017283mg | | | | | | | | |
| | PE = 3 SV = 1 - | | | | | | | | |
| | [V4UF13_9ROSI] | | | | | | | | |
| A0A067GB09 | Starch synthase, | 6.55 | 6 | 2 | 3 | 5 | 580 | 63.9 | 8.78 |
| | chloroplastic/amyloplastic | | | | | | | | |
| | OS = Citrus sinensis | | | | | | | | |
| | GN = CISIN_1g008028mg | | | | | | | | |
| | PE = 3 SV = 1 - | | | | | | | | |
| | [A0A067GB09_CITSI] | | | | | | | | |
| Q198M7 | Succinate dehydrogenase | 13.84 | 1 | 2 | 2 | 2 | 159 | 17.6 | 8.81 |
| | (Fragment) OS = Mangifera | | | | | | | | |
| | indica PE = 2 SV = 1 - | | | | | | | | |
| | [Q198M7_MANIN] | | | | | | | | |
| V4SED1 | Succinate dehydrogenase | 8.07 | 3 | 3 | 3 | 3 | 632 | 69.6 | 6.49 |
| | [ubiquinone] flavoprotein | | | | | | | | |
| | subunit, mitochondrial | | | | | | | | |
| | OS = Citrus clementina | | | | | | | | |
| | GN = CICLE_v10025149mg | | | | | | | | |
| | PE = 3 SV = 1 - | | | | | | | | |
| | [V4SED1_9ROSI] | | | | | | | | |
| A0A067FB34 | Succinyl-CoA ligase subunit | 7.82 | 3 | 3 | 3 | 3 | 422 | 45.2 | 6.32 |
| | beta OS = Citrus sinensis | | | | | | | | |
| | GN = CISIN_1g014588mg | | | | | | | | |
| | PE = 3 SV = 1 - | | | | | | | | |
| | [A0A067FB34_CITSI] | | | | | | | | |
| I7EKT3 | Sucrose phosphate synthase | 6.99 | 20 | 7 | 7 | 8 | 1045 | 117.3 | 6.44 |
| | OS = Litchi chinensis | | | | | | | | |
| | GN = SPS PE = 2 SV = 1 - | | | | | | | | |
| | [I7EKT3_LITCN] | | | | | | | | |
| A0A0D5CT73 | Sucrose synthase | 3.45 | 14 | 3 | 3 | 3 | 811 | 92.6 | 6.33 |
| | OS = Dimocarpus longan | | | | | | | | |
| | GN = SS-3 PE = 2 SV = 1 - | | | | | | | | |
| | [A0A0D5CT73_9ROSI] | | | | | | | | |
| V4SB29 | Superoxide dismutase | 6.44 | 5 | 1 | 1 | 1 | 233 | 23.6 | 7.18 |
| | [Cu - Zn] OS = Citrus | | | | | | | | |
| | clementina | | | | | | | | |
| | GN = CICLE_v10029196mg | | | | | | | | |
| | PE = 3 SV = 1 - | | | | | | | | |
| | [V4SB29_9ROSI] | | | | | | | | |
| B2BDZ8 | Superoxide dismutase | 11.30 | 13 | 2 | 2 | 3 | 230 | 25.7 | 7.66 |
| | OS = Pistacia vera PE = 3 | | | | | | | | |
| | SV = 1 - [B2BDZ8_PISVE] | | | | | | | | |
| A0A067F9M5 | T-complex protein 1 subunit | 6.92 | 2 | 3 | 3 | 3 | 535 | 57.6 | 7.44 |
| | delta OS = Citrus sinensis | | | | | | | | |
| | GN = CISIN_1g009431mg | | | | | | | | |
| | PE = 3 SV = 1 - | | | | | | | | |
| | [A0A067F9M5_CITSI] | | | | | | | | |
| V4S279 | T-complex protein 1 subunit | 10.95 | 10 | 6 | 6 | 7 | 557 | 60.7 | 6.13 |
| | gamma OS = Citrus | | | | | | | | |
| | clementina | | | | | | | | |
| | GN = CICLE_v10025301mg | | | | | | | | |
| | PE = 3 SV = 1 - | | | | | | | | |
| | [V4S279_9ROSI] | | | | | | | | |
| D2KU75 | Thaumatin-like protein | 3.63 | 2 | 1 | 1 | 2 | 248 | 26.7 | 7.91 |
| | OS = Citrus jambhiri | | | | | | | | |
| | GN = RlemTLP PE = 2 SV = 1 - | | | | | | | | |
| | [D2KU75_CITJA] | | | | | | | | |
| A1ECJ9 | Thioredoxin OS = Citrus | 8.41 | 4 | 2 | 2 | 3 | 107 | 11.9 | 5.24 |
| | hybrid cultivar PE = 2 SV = 1 - | | | | | | | | |
| | [A1ECJ9_9ROSI] | | | | | | | | |
| B7UCK2 | Transaldolase | 12.50 | 2 | 1 | 5 | 5 | 440 | 47.9 | 6.80 |
| | OS = Dimocarpus longan | | | | | | | | |
| | PE = 2 SV = 1 - | | | | | | | | |
| | [B7UCK2_9ROSI] | | | | | | | | |
| C3VIC2 | Translation elongation factor | 6.94 | 9 | 1 | 2 | 4 | 418 | 47.6 | 6.29 |
| | OS = Citrus maxima PE = 2 | | | | | | | | |
| | SV = 1 - [C3VIC2_CITMA] | | | | | | | | |
| F2XX46 | Translationally controlled | 14.88 | 3 | 1 | 2 | 3 | 168 | 18.8 | 4.67 |
| | tumor protein OS = Litchi | | | | | | | | |
| | chinensis GN = TCTP PE = 2 | | | | | | | | |
| | SV = 1 - [F2XX46_LITCN] | | | | | | | | |
| V4RQS1 | Triosephosphate isomerase | 7.24 | 5 | 1 | 2 | 4 | 304 | 32.7 | 7.36 |
| | OS = Citrus clementina | | | | | | | | |
| | GN = CICLE_v10028924mg | | | | | | | | |
| | PE = 3 SV = 1 - | | | | | | | | |
| | [V4RQS1_9ROSI] | | | | | | | | |
| V4SGV7 | Triosephosphate isomerase | 24.90 | 5 | 1 | 4 | 8 | 253 | 27.2 | 6.00 |
| | OS = Citrus clementina | | | | | | | | |
| | GN = CICLE_v10029115mg | | | | | | | | |
| | PE = 3 SV = 1 - | | | | | | | | |
| | [V4SGV7_9ROSI] | | | | | | | | |
| V4T6F5 | Triosephosphate isomerase | 28.29 | 8 | 1 | 5 | 10 | 251 | 27.0 | 5.68 |
| | OS = Citrus clementina | | | | | | | | |
| | GN = CICLE_v10032537mg | | | | | | | | |
| | PE = 3 SV = 1 - | | | | | | | | |
| | [V4T6F5_9ROSI] | | | | | | | | |
| B9VRG4 | Triosephosphate isomerase | 20.08 | 1 | 1 | 3 | 5 | 254 | 27.2 | 6.35 |
| | OS = Dimocarpus longan | | | | | | | | |
| | PE = 2 SV = 2 - | | | | | | | | |
| | [B9VRG4_9ROSI] | | | | | | | | |
| V4T0G7 | Uncharacterized protein | 7.53 | 3 | 2 | 5 | 5 | 744 | 80.3 | 5.80 |
| | (Fragment) OS = Citrus | | | | | | | | |
| | clementina | | | | | | | | |
| | GN = CICLE_v10003758mg | | | | | | | | |
| | PE = 3 SV = 1 - | | | | | | | | |
| | [V4T0G7_9ROSI] | | | | | | | | |
| V4S7C4 | Uncharacterized protein | 45.00 | 2 | 2 | 3 | 3 | 80 | 8.2 | 9.72 |
| | (Fragment) OS = Citrus | | | | | | | | |
| | clementina | | | | | | | | |
| | GN = CICLE_v100061991mg | | | | | | | | |
| | PE = 4 SV = 1 - | | | | | | | | |
| | [V4S7C4_9ROSI] | | | | | | | | |
| V4U3S4 | Uncharacterized protein | 14.41 | 4 | 2 | 2 | 2 | 118 | 12.9 | 7.33 |
| | (Fragment) OS = Citrus | | | | | | | | |
| | clementina | | | | | | | | |
| | GN = CICLE_v10006918mg | | | | | | | | |
| | PE = 4 SV = 1 - | | | | | | | | |
| | [V4U3S4_9ROSI] | | | | | | | | |
| V4TSD8 | Uncharacterized protein | 1.52 | 3 | 1 | 1 | 1 | 592 | 68.5 | 6.02 |
| | (Fragment) OS = Citrus | | | | | | | | |
| | clementina | | | | | | | | |
| | GN = CICLE_v100076092mg | | | | | | | | |
| | PE = 3 SV = 1 - | | | | | | | | |
| | [V4TSD8_9ROSI] | | | | | | | | |
| V4UMC7 | Uncharacterized protein | 7.14 | 1 | 1 | 1 | 1 | 98 | 11.4 | 5.26 |
| | (Fragment) OS = Citrus | | | | | | | | |
| | clementina | | | | | | | | |
| | GN = CICLE_v100076132mg | | | | | | | | |
| | PE = 4 SV = 1 - | | | | | | | | |
| | [V4UMC7_9ROSI] | | | | | | | | |
| V4SZ50 | Uncharacterized protein | 2.04 | 3 | 1 | 1 | 1 | 636 | 70.8 | 7.78 |
| | (Fragment) OS = Citrus | | | | | | | | |
| | clementina | | | | | | | | |
| | GN = CICLE_v100111191mg | | | | | | | | |
| | PE = 3 SV = 1 - | | | | | | | | |
| | [V4SZ50_9ROSI] | | | | | | | | |
| V4SW04 | Uncharacterized protein | 2.53 | 5 | 1 | 1 | 1 | 435 | 47.6 | 7.59 |
| | (Fragment) OS = Citrus | | | | | | | | |
| | clementina | | | | | | | | |
| | GN = CICLE_v100114061mg | | | | | | | | |
| | PE = 4 SV = 1 - | | | | | | | | |
| | [V4SW04_9ROSI] | | | | | | | | |
| V4SX65 | Uncharacterized protein | 11.45 | 6 | 2 | 2 | 2 | 166 | 18.0 | 9.72 |
| | (Fragment) OS = Citrus | | | | | | | | |
| | clementina | | | | | | | | |
| | GN = CICLE_v10013826mg | | | | | | | | |
| | PE = 3 SV = 1 - | | | | | | | | |
| | [V4SX65_9ROSI] | | | | | | | | |
| V4W9G2 | Uncharacterized protein | 7.38 | 3 | 1 | 1 | 1 | 149 | 16.2 | 10.70 |
| | (Fragment) OS = Citrus | | | | | | | | |
| | clementina | | | | | | | | |
| | GN = CICLE_v100179502mg | | | | | | | | |
| | PE = 3 SV = 1 - | | | | | | | | |
| | [V4W9G2_9ROSI] | | | | | | | | |
| V4TKQ5 | Uncharacterized protein | 10.87 | 4 | 2 | 2 | 2 | 230 | 25.3 | 9.14 |
| | (Fragment) OS = Citrus | | | | | | | | |
| | clementina | | | | | | | | |
| | GN = CICLE_v100211692mg | | | | | | | | |
| | PE = 4 SV = 1 - | | | | | | | | |
| | [V4TKQ5_9ROSI] | | | | | | | | |
| V4U6L5 | Uncharacterized protein | 16.67 | 3 | 2 | 2 | 2 | 108 | 11.5 | 5.29 |
| | (Fragment) OS = Citrus | | | | | | | | |
| | clementina | | | | | | | | |
| | GN = CICLE_v100223782mg | | | | | | | | |
| | PE = 3 SV = 1 - | | | | | | | | |
| | [V4U6L5_9ROSI] | | | | | | | | |
| V4TMM8 | Uncharacterized protein | 8.05 | 8 | 1 | 1 | 1 | 87 | 9.5 | 8.51 |
| | (Fragment) OS = Citrus | | | | | | | | |
| | clementina | | | | | | | | |
| | GN = CICLE_v100239431mg | | | | | | | | |
| | PE = 4 SV = 1 - | | | | | | | | |
| | [V4TMM8_9ROSI] | | | | | | | | |
| V4TVE5 | Uncharacterized protein | 6.13 | 2 | 1 | 1 | 2 | 212 | 23.9 | 8.81 |
| | (Fragment) OS = Citrus | | | | | | | | |
| | clementina | | | | | | | | |
| | GN = CICLE_v10024356mg | | | | | | | | |
| | PE = 3 SV = 1 - | | | | | | | | |
| | [V4TVE5_9ROSI] | | | | | | | | |
| V4RVH7 | Uncharacterized protein | 3.07 | 2 | 1 | 1 | 1 | 424 | 47.6 | 6.07 |
| | (Fragment) OS = Citrus | | | | | | | | |
| | clementina | | | | | | | | |
| | GN = CICLE_v100254672mg | | | | | | | | |
| | PE = 4 SV = 1 - | | | | | | | | |
| | [V4RVH7_9ROSI] | | | | | | | | |
| V4SJC8 | Uncharacterized protein | 1.51 | 1 | 1 | 1 | 1 | 529 | 62.3 | 5.44 |
| | (Fragment) OS = Citrus | | | | | | | | |
| | clementina | | | | | | | | |
| | GN = CICLE_v10027019mg | | | | | | | | |
| | PE = 4 SV = 1 - | | | | | | | | |
| | [V4SJC8_9ROSI] | | | | | | | | |
| V4S245 | Uncharacterized protein | 8.70 | 5 | 2 | 2 | 2 | 207 | 23.5 | 7.59 |
| | (Fragment) OS = Citrus | | | | | | | | |
| | clementina | | | | | | | | |
| | GN = CICLE_v10027333mg | | | | | | | | |
| | PE = 4 SV = 1 - | | | | | | | | |
| | [V4S245_9ROSI] | | | | | | | | |
| V4T803 | Uncharacterized protein | 14.78 | 9 | 1 | 1 | 1 | 115 | 12.8 | 8.37 |
| | (Fragment) OS = Citrus | | | | | | | | |
| | clementina | | | | | | | | |
| | GN = CICLE_v100329312mg | | | | | | | | |
| | PE = 4 SV = 1 - | | | | | | | | |
| | [V4T803_9ROSI] | | | | | | | | |
| V4TC62 | Uncharacterized protein | 10.47 | 9 | 1 | 1 | 1 | 86 | 10.0 | 10.39 |
| | (Fragment) OS = Citrus | | | | | | | | |
| | clementina | | | | | | | | |
| | GN = CICLE_v10033333mg | | | | | | | | |
| | PE = 4 SV = 1 - | | | | | | | | |
| | [V4TC62_9ROSI] | | | | | | | | |
| A0A067G5Y6 | Uncharacterized protein | 1.76 | 3 | 1 | 1 | 1 | 681 | 76.0 | 6.80 |
| | (Fragment) OS = Citrus | | | | | | | | |
| | sinensis | | | | | | | | |
| | GN = CISIN_1g0002071mg | | | | | | | | |
| | PE = 4 SV = 1 - | | | | | | | | |
| | [A0A067G5Y6_CITSI] | | | | | | | | |
| A0A067H3P6 | Uncharacterized protein | 1.53 | 7 | 1 | 1 | 1 | 848 | 94.7 | 5.29 |
| | (Fragment) OS = Citrus | | | | | | | | |
| | sinensis | | | | | | | | |
| | GN = CISIN_1g0019001mg | | | | | | | | |
| | PE = 4 SV = 1 - | | | | | | | | |
| | [A0A067H3P6_CITSI] | | | | | | | | |
| A0A067E2Y6 | Uncharacterized protein | 4.90 | 3 | 1 | 1 | 1 | 347 | 39.4 | 4.72 |
| | (Fragment) OS = Citrus | | | | | | | | |
| | sinensis | | | | | | | | |
| | GN = CISIN_1g0026962mg | | | | | | | | |
| | PE = 4 SV = 1 - | | | | | | | | |
| | [A0A067E2Y6_CITSI] | | | | | | | | |
| A0A067ED12 | Uncharacterized protein | 1.78 | 4 | 1 | 1 | 1 | 505 | 55.7 | 4.81 |
| | (Fragment) OS = Citrus | | | | | | | | |
| | sinensis | | | | | | | | |
| | GN = CISIN_1g0029002mg | | | | | | | | |
| | PE = 4 SV = 1 - | | | | | | | | |
| | [A0A067ED12_CITSI] | | | | | | | | |
| A0A067F4M4 | Uncharacterized protein | 1.61 | 7 | 1 | 1 | 1 | 620 | 71.1 | 5.76 |
| | (Fragment) OS = Citrus | | | | | | | | |
| | sinensis | | | | | | | | |
| | GN = CISIN_1g0032301mg | | | | | | | | |
| | PE = 4 SV = 1 - | | | | | | | | |
| | [A0A067F4M4_CITSI] | | | | | | | | |
| A0A067H0F4 | Uncharacterized protein | 3.90 | 6 | 1 | 1 | 1 | 231 | 26.1 | 8.07 |
| | (Fragment) OS = Citrus | | | | | | | | |
| | sinensis | | | | | | | | |
| | GN = CISIN_1g0039772mg | | | | | | | | |
| | PE = 4 SV = 1 - | | | | | | | | |
| | [A0A067H0F4_CITSI] | | | | | | | | |
| A0A067FBC0 | Uncharacterized protein | 23.39 | 2 | 1 | 1 | 1 | 124 | 13.3 | 7.46 |
| | (Fragment) OS = Citrus | | | | | | | | |
| | sinensis | | | | | | | | |
| | GN = CISIN_1g0040342mg | | | | | | | | |
| | PE = 4 SV = 1 - | | | | | | | | |
| | [A0A067FBC0_CITSI] | | | | | | | | |
| A0A067E852 | Uncharacterized protein | 5.87 | 16 | 4 | 4 | 4 | 682 | 71.4 | 6.00 |
| | (Fragment) OS = Citrus | | | | | | | | |
| | sinensis | | | | | | | | |
| | GN = CISIN_1g0042302mg | | | | | | | | |
| | PE = 3 SV = 1 - | | | | | | | | |
| | [A0A067E852_CITSI] | | | | | | | | |
| A0A067EPJ8 | Uncharacterized protein | 5.10 | 6 | 1 | 1 | 1 | 353 | 39.9 | 5.72 |
| | (Fragment) OS = Citrus | | | | | | | | |
| | sinensis | | | | | | | | |
| | GN = CISIN_1g0042382mg | | | | | | | | |
| | PE = 4 SV = 1 - | | | | | | | | |
| | [A0A067EPJ8_CITSI] | | | | | | | | |
| A0A067GV09 | Uncharacterized protein | 7.89 | 4 | 2 | 2 | 2 | 266 | 30.9 | 7.15 |
| | (Fragment) OS = Citrus | | | | | | | | |
| | sinensis | | | | | | | | |
| | GN = CISIN_1g0066801mg | | | | | | | | |
| | PE = 3 SV = 1 - | | | | | | | | |
| | [A0A067GV09_CITSI] | | | | | | | | |
| A0A067GV04 | Uncharacterized protein | 3.55 | 2 | 1 | 1 | 1 | 310 | 34.9 | 8.10 |
| | (Fragment) OS = Citrus | | | | | | | | |
| | sinensis | | | | | | | | |
| | GN = CISIN_1g0066802mg | | | | | | | | |
| | PE = 4 SV = 1 - | | | | | | | | |
| | [A0A067GV04_CITSI] | | | | | | | | |
| A0A067G5Y0 | Uncharacterized protein | 2.18 | 7 | 1 | 1 | 1 | 413 | 44.5 | 8.12 |
| | (Fragment) OS = Citrus | | | | | | | | |
| | sinensis | | | | | | | | |
| | GN = CISIN_1g0071512mg | | | | | | | | |
| | PE = 3 SV = 1 - | | | | | | | | |
| | [A0A067G5Y0_CITSI] | | | | | | | | |
| A0A067F5H6 | Uncharacterized protein | 23.19 | 7 | 2 | 2 | 2 | 69 | 7.8 | 7.99 |
| | (Fragment) OS = Citrus | | | | | | | | |
| | sinensis | | | | | | | | |
| | GN = CISIN_1g0075041mg | | | | | | | | |
| | PE = 4 SV = 1 - | | | | | | | | |
| | [A0A067F5H6_CITSI] | | | | | | | | |
| A0A067FGI1 | Uncharacterized protein | 23.44 | 2 | 1 | 1 | 1 | 64 | 6.7 | 10.10 |
| | (Fragment) OS = Citrus | | | | | | | | |
| | sinensis | | | | | | | | |
| | GN = CISIN_1g0081812mg | | | | | | | | |
| | PE = 4 SV = 1 - | | | | | | | | |
| | [A0A067FGI1_CITSI] | | | | | | | | |
| A0A067EHF7 | Uncharacterized protein | 3.28 | 2 | 1 | 1 | 1 | 396 | 43.7 | 9.19 |
| | (Fragment) OS = Citrus | | | | | | | | |
| | sinensis | | | | | | | | |
| | GN = CISIN_1g0088521mg | | | | | | | | |
| | PE = 4 SV = 1 - | | | | | | | | |
| | [A0A067EHF7_CITSI] | | | | | | | | |
| A0A067E492 | Uncharacterized protein | 3.73 | 3 | 2 | 2 | 2 | 402 | 43.0 | 9.14 |
| | (Fragment) OS = Citrus | | | | | | | | |
| | sinensis | | | | | | | | |
| | GN = CISIN_1g0099912mg | | | | | | | | |
| | PE = 4 SV = 1 - | | | | | | | | |
| | [A0A067E492_CITSI] | | | | | | | | |
| A0A067FLJ4 | Uncharacterized protein | 5.86 | 14 | 1 | 1 | 2 | 239 | 27.4 | 5.41 |
| | (Fragment) OS = Citrus | | | | | | | | |
| | sinensis | | | | | | | | |
| | GN = CISIN_1g0105291mg | | | | | | | | |
| | PE = 4 SV = 1 - | | | | | | | | |
| | [A0A067FLJ4_CITSI] | | | | | | | | |
| A0A067FQW4 | Uncharacterized protein | 3.01 | 2 | 1 | 1 | 1 | 299 | 32.6 | 5.33 |
| | (Fragment) OS = Citrus | | | | | | | | |
| | sinensis | | | | | | | | |
| | GN = CISIN_1g0115531mg | | | | | | | | |
| | PE = 4 SV = 1 - | | | | | | | | |
| | [A0A067FQW4_CITSI] | | | | | | | | |
| A0A067GN67 | Uncharacterized protein | 3.76 | 2 | 1 | 1 | 1 | 346 | 38.4 | 7.88 |
| | (Fragment) OS = Citrus | | | | | | | | |
| | sinensis | | | | | | | | |
| | GN = CISIN_1g0117072mg | | | | | | | | |
| | PE = 4 SV = 1 - | | | | | | | | |
| | [A0A067GN67_CITSI] | | | | | | | | |
| A0A067DY38 | Uncharacterized protein | 8.00 | 2 | 1 | 1 | 1 | 200 | 22.0 | 5.58 |
| | (Fragment) OS = Citrus | | | | | | | | |
| | sinensis | | | | | | | | |
| | GN = CISIN_1g0120981mg | | | | | | | | |
| | PE = 3 SV = 1 - | | | | | | | | |
| | [A0A067DY38_CITSI] | | | | | | | | |
| A0A067HC49 | Uncharacterized protein | 9.45 | 6 | 1 | 1 | 1 | 127 | 14.6 | 6.54 |
| | (Fragment) OS = Citrus | | | | | | | | |
| | sinensis | | | | | | | | |
| | GN = CISIN_1g0137322mg | | | | | | | | |
| | PE = 4 SV = 1 - | | | | | | | | |
| | [A0A067HC49_CITSI] | | | | | | | | |
| A0A067EP63 | Uncharacterized protein | 11.83 | 3 | 1 | 1 | 1 | 93 | 10.9 | 5.83 |
| | (Fragment) OS = Citrus | | | | | | | | |
| | sinensis | | | | | | | | |
| | GN = CISIN_1g0144531mg | | | | | | | | |
| | PE = 4 SV = 1 - | | | | | | | | |
| | [A0A067EP63_CITSI] | | | | | | | | |
| A0A067E1Z7 | Uncharacterized protein | 6.90 | 3 | 1 | 1 | 1 | 203 | 23.9 | 9.72 |
| | (Fragment) OS = Citrus | | | | | | | | |
| | sinensis | | | | | | | | |
| | GN = CISIN_1g0151221mg | | | | | | | | |
| | PE = 4 SV = 1 - | | | | | | | | |
| | [A0A067E1Z7_CITSI] | | | | | | | | |
| A0A067FQZ8 | Uncharacterized protein | 3.76 | 5 | 1 | 1 | 1 | 319 | 35.5 | 6.09 |
| | (Fragment) OS = Citrus | | | | | | | | |
| | sinensis | | | | | | | | |
| | GN = CISIN_1g0152591mg | | | | | | | | |
| | PE = 4 SV = 1 - | | | | | | | | |
| | [A0A067FQZ8_CITSI] | | | | | | | | |
| A0A067GPK2 | Uncharacterized protein | 2.34 | 4 | 1 | 1 | 1 | 384 | 43.4 | 5.35 |
| | (Fragment) OS = Citrus | | | | | | | | |
| | sinensis | | | | | | | | |
| | GN = CISIN_1g0159921mg | | | | | | | | |
| | PE = 4 SV = 1 - | | | | | | | | |
| | [A0A067GPK2_CITSI] | | | | | | | | |
| A0A067EMA9 | Uncharacterized protein | 4.61 | 3 | 1 | 1 | 1 | 304 | 32.9 | 5.44 |
| | (Fragment) OS = Citrus | | | | | | | | |
| | sinensis | | | | | | | | |
| | GN = CISIN_1g0162802mg | | | | | | | | |
| | PE = 4 SV = 1 - | | | | | | | | |
| | [A0A067EMA9_CITSI] | | | | | | | | |
| A0A067G3B7 | Uncharacterized protein | 3.25 | 5 | 1 | 1 | 1 | 277 | 28.8 | 5.39 |
| | (Fragment) OS = Citrus | | | | | | | | |
| | sinensis | | | | | | | | |
| | GN = CISIN_1g0163871mg | | | | | | | | |
| | PE = 4 SV = 1 - | | | | | | | | |
| | [A0A067G3B7_CITSI] | | | | | | | | |
| A0A067FAA4 | Uncharacterized protein | 6.13 | 6 | 1 | 1 | 1 | 163 | 17.6 | 6.77 |
| | (Fragment) OS = Citrus | | | | | | | | |
| | sinensis | | | | | | | | |
| | GN = CISIN_1g0174262mg | | | | | | | | |
| | PE = 4 SV = 1 - | | | | | | | | |
| | [A0A067FAA4_CITSI] | | | | | | | | |
| A0A067FE94 | Uncharacterized protein | 7.10 | 2 | 2 | 2 | 2 | 324 | 36.8 | 7.59 |
| | (Fragment) OS = Citrus | | | | | | | | |
| | sinensis | | | | | | | | |
| | GN = CISIN_1g0178621mg | | | | | | | | |
| | PE = 4 SV = 1 - | | | | | | | | |
| | [A0A067FE94_CITSI] | | | | | | | | |
| A0A067GB25 | Uncharacterized protein | 4.84 | 3 | 1 | 1 | 1 | 289 | 31.0 | 7.68 |
| | (Fragment) OS = Citrus | | | | | | | | |
| | sinensis | | | | | | | | |
| | GN = CISIN_1g0191991mg | | | | | | | | |
| | PE = 3 SV = 1 - | | | | | | | | |
| | [A0A067GB25_CITSI] | | | | | | | | |
| A0A067EQ02 | Uncharacterized protein | 5.33 | 4 | 1 | 1 | 1 | 225 | 24.1 | 5.81 |
| | (Fragment) OS = Citrus | | | | | | | | |
| | sinensis | | | | | | | | |
| | GN = CISIN_1g0222852mg | | | | | | | | |
| | PE = 3 SV = 1 - | | | | | | | | |
| | [A0A067EQ02_CITSI] | | | | | | | | |
| A0A067DSY2 | Uncharacterized protein | 8.33 | 10 | 2 | 2 | 2 | 264 | 29.9 | 9.47 |
| | (Fragment) OS = Citrus | | | | | | | | |
| | sinensis | | | | | | | | |
| | GN = CISIN_1g0225141mg | | | | | | | | |
| | PE = 3 SV = 1 - | | | | | | | | |
| | [A0A067DSY2_CITSI] | | | | | | | | |
| A0A067GY14 | Uncharacterized protein | 7.10 | 2 | 1 | 1 | 1 | 183 | 19.7 | 5.58 |
| | (Fragment) OS = Citrus | | | | | | | | |
| | sinensis | | | | | | | | |
| | GN = CISIN_1g0242241mg | | | | | | | | |
| | PE = 4 SV = 1 - | | | | | | | | |
| | [A0A067GY14_CITSI] | | | | | | | | |
| A0A067H1C8 | Uncharacterized protein | 9.28 | 4 | 1 | 1 | 1 | 97 | 10.6 | 9.82 |
| | (Fragment) OS = Citrus | | | | | | | | |
| | sinensis | | | | | | | | |
| | GN = CISIN_1g0249171mg | | | | | | | | |
| | PE = 4 SV = 1 - | | | | | | | | |
| | [A0A067H1C8_CITSI] | | | | | | | | |
| A0A067GJW3 | Uncharacterized protein | 6.64 | 3 | 1 | 1 | 1 | 241 | 26.4 | 5.30 |
| | (Fragment) OS = Citrus | | | | | | | | |
| | sinensis | | | | | | | | |
| | GN = CISIN_1g0258351mg | | | | | | | | |
| | PE = 4 SV = 1 - | | | | | | | | |
| | [A0A067GJW3_CITSI] | | | | | | | | |
| A0A067DNI6 | Uncharacterized protein | 5.29 | 3 | 1 | 1 | 1 | 170 | 19.4 | 5.14 |
| | (Fragment) OS = Citrus | | | | | | | | |
| | sinensis | | | | | | | | |
| | GN = CISIN_1g0264261mg | | | | | | | | |
| | PE = 4 SV = 1 - | | | | | | | | |
| | [A0A067DNI6_CITSI] | | | | | | | | |
| A0A067GCQ8 | Uncharacterized protein | 11.94 | 3 | 1 | 1 | 1 | 67 | 8.1 | 5.58 |
| | (Fragment) OS = Citrus | | | | | | | | |
| | sinensis | | | | | | | | |
| | GN = CISIN_1g0269491mg | | | | | | | | |
| | PE = 4 SV = 1 - | | | | | | | | |
| | [A0A067GCQ8_CITSI] | | | | | | | | |
| A0A067F5R2 | Uncharacterized protein | 6.51 | 4 | 1 | 1 | 1 | 169 | 19.0 | 5.08 |
| | (Fragment) OS = Citrus | | | | | | | | |
| | sinensis | | | | | | | | |
| | GN = CISIN_1g0284552mg | | | | | | | | |
| | PE = 4 SV = 1 - | | | | | | | | |
| | [A0A067F5R2_CITSI] | | | | | | | | |
| A0A067DSS7 | Uncharacterized protein | 23.17 | 5 | 1 | 1 | 1 | 82 | 9.2 | 9.82 |
| | (Fragment) OS = Citrus | | | | | | | | |
| | sinensis | | | | | | | | |
| | GN = CISIN_1g0316532mg | | | | | | | | |
| | PE = 3 SV = 1 - | | | | | | | | |
| | [A0A067DSS7_CITSI] | | | | | | | | |
| A0A067GXV8 | Uncharacterized protein | 19.23 | 3 | 1 | 1 | 1 | 52 | 5.6 | 9.06 |
| | (Fragment) OS = Citrus | | | | | | | | |
| | sinensis | | | | | | | | |
| | GN = CISIN_1g0330612mg | | | | | | | | |
| | PE = 4 SV = 1 - | | | | | | | | |
| | [A0A067GXV8_CITSI] | | | | | | | | |
| A0A067FGY6 | Uncharacterized protein | 12.12 | 2 | 1 | 1 | 1 | 99 | 11.3 | 10.07 |
| | (Fragment) OS = Citrus | | | | | | | | |
| | sinensis | | | | | | | | |
| | GN = CISIN_1g0334821mg | | | | | | | | |
| | PE = 3 SV = 1 - | | | | | | | | |
| | [A0A067FGY6_CITSI] | | | | | | | | |
| A0A067EMY2 | Uncharacterized protein | 17.24 | 7 | 1 | 1 | 1 | 58 | 5.9 | 6.51 |
| | (Fragment) OS = Citrus | | | | | | | | |
| | sinensis | | | | | | | | |
| | GN = CISIN_1g0339621mg | | | | | | | | |
| | PE = 4 SV = 1 - | | | | | | | | |
| | [A0A067EMY2_CITSI] | | | | | | | | |
| A0A067F4Z0 | Uncharacterized protein | 47.83 | 4 | 1 | 1 | 1 | 23 | 2.7 | 12.02 |
| | (Fragment) OS = Citrus | | | | | | | | |
| | sinensis | | | | | | | | |
| | GN = CISIN_1g0346512mg | | | | | | | | |
| | PE = 4 SV = 1 - | | | | | | | | |
| | [A0A067F4Z0_CITSI] | | | | | | | | |
| A0A067EJ54 | Uncharacterized protein | 2.53 | 3 | 1 | 1 | 1 | 316 | 36.6 | 6.30 |
| | (Fragment) OS = Citrus | | | | | | | | |
| | sinensis | | | | | | | | |
| | GN = CISIN_1g036278mg | | | | | | | | |
| | PE = 4 SV = 1 - | | | | | | | | |
| | [A0A067EJ54_CITSI] | | | | | | | | |
| A0A067GPX4 | Uncharacterized protein | 7.38 | 3 | 1 | 1 | 1 | 122 | 13.8 | 4.84 |
| | (Fragment) OS = Citrus | | | | | | | | |
| | sinensis | | | | | | | | |
| | GN = CISIN_1g036836mg | | | | | | | | |
| | PE = 4 SV = 1 - | | | | | | | | |
| | [A0A067GPX4_CITSI] | | | | | | | | |
| A0A067G4J1 | Uncharacterized protein | 6.23 | 6 | 1 | 1 | 1 | 273 | 29.4 | 8.59 |
| | (Fragment) OS = Citrus | | | | | | | | |
| | sinensis | | | | | | | | |
| | GN = CISIN_1g036999mg | | | | | | | | |
| | PE = 4 SV = 1 - | | | | | | | | |
| | [A0A067G4J1_CITSI] | | | | | | | | |
| A0A067FUU5 | Uncharacterized protein | 14.65 | 3 | 2 | 2 | 3 | 198 | 21.7 | 10.39 |
| | (Fragment) OS = Citrus | | | | | | | | |
| | sinensis | | | | | | | | |
| | GN = CISIN_1g037430mg | | | | | | | | |
| | PE = 4 SV = 1 - | | | | | | | | |
| | [A0A067FUU5_CITSI] | | | | | | | | |
| A0A067E1J8 | Uncharacterized protein | 5.14 | 8 | 2 | 2 | 2 | 467 | 54.9 | 5.82 |
| | (Fragment) OS = Citrus | | | | | | | | |
| | sinensis | | | | | | | | |
| | GN = CISIN_1g0374612mg | | | | | | | | |
| | PE = 4 SV = 1 - | | | | | | | | |
| | [A0A067E1J8_CITSI] | | | | | | | | |
| A0A067GTK7 | Uncharacterized protein | 0.70 | 3 | 1 | 1 | 1 | 1293 | 139.4 | 6.07 |
| | (Fragment) OS = Citrus | | | | | | | | |
| | sinensis | | | | | | | | |
| | GN = CISIN_1g0397901mg | | | | | | | | |
| | PE = 4 SV = 1 - | | | | | | | | |
| | [A0A067GTK7_CITSI] | | | | | | | | |
| A0A067F7N1 | Uncharacterized protein | 4.78 | 2 | 1 | 1 | 4 | 272 | 29.9 | 5.77 |
| | (Fragment) OS = Citrus | | | | | | | | |
| | sinensis | | | | | | | | |
| | GN = CISIN_1g039897mg | | | | | | | | |
| | PE = 4 SV = 1 - | | | | | | | | |
| | [A0A067F7N1_CITSI] | | | | | | | | |
| A0A067DCF3 | Uncharacterized protein | 4.88 | 2 | 1 | 1 | 2 | 164 | 18.5 | 6.20 |
| | (Fragment) OS = Citrus | | | | | | | | |
| | sinensis | | | | | | | | |
| | GN = CISIN_1g040329mg | | | | | | | | |
| | PE = 4 SV = 1 - | | | | | | | | |
| | [A0A067DCF3_CITSI] | | | | | | | | |
| A0A067DHH9 | Uncharacterized protein | 7.81 | 2 | 1 | 1 | 1 | 128 | 15.0 | 8.72 |
| | (Fragment) OS = Citrus | | | | | | | | |
| | sinensis | | | | | | | | |
| | GN = CISIN_1g0413981mg | | | | | | | | |
| | PE = 4 SV = 1 - | | | | | | | | |
| | [A0A067DHH9_CITSI] | | | | | | | | |
| A0A067FYQ2 | Uncharacterized protein | 15.05 | 10 | 3 | 10 | 11 | 784 | 87.2 | 5.26 |
| | (Fragment) OS = Citrus | | | | | | | | |
| | sinensis | | | | | | | | |
| | GN = CISIN_1g042193mg | | | | | | | | |
| | PE = 3 SV = 1 - | | | | | | | | |
| | [A0A067FYQ2_CITSI] | | | | | | | | |
| A0A067DF04 | Uncharacterized protein | 9.78 | 2 | 1 | 1 | 1 | 92 | 10.5 | 6.52 |
| | (Fragment) OS = Citrus | | | | | | | | |
| | sinensis | | | | | | | | |
| | GN = CISIN_1g042638mg | | | | | | | | |
| | PE = 4 SV = 1 - | | | | | | | | |
| | [A0A067DF04_CITSI] | | | | | | | | |
| A0A067EY68 | Uncharacterized protein | 5.36 | 2 | 2 | 2 | 2 | 336 | 38.4 | 5.68 |
| | (Fragment) OS = Citrus | | | | | | | | |
| | sinensis | | | | | | | | |
| | GN = CISIN_1g0437802mg | | | | | | | | |
| | PE = 4 SV = 1 - | | | | | | | | |
| | [A0A067EY68_CITSI] | | | | | | | | |
| A0A067EPK9 | Uncharacterized protein | 10.81 | 2 | 1 | 1 | 2 | 74 | 8.5 | 9.60 |
| | (Fragment) OS = Citrus | | | | | | | | |
| | sinensis | | | | | | | | |
| | GN = CISIN_1g043909mg | | | | | | | | |
| | PE = 4 SV = 1 - | | | | | | | | |
| | [A0A067EPK9_CITSI] | | | | | | | | |
| A0A067GPV7 | Uncharacterized protein | 7.03 | 2 | 1 | 1 | 1 | 128 | 13.9 | 4.54 |
| | (Fragment) OS = Citrus | | | | | | | | |
| | sinensis | | | | | | | | |
| | GN = CISIN_1g044104mg | | | | | | | | |
| | PE = 3 SV = 1 - | | | | | | | | |
| | [A0A067GPV7_CITSI] | | | | | | | | |
| A0A067F810 | Uncharacterized protein | 5.47 | 6 | 1 | 1 | 2 | 201 | 23.0 | 6.70 |
| | (Fragment) OS = Citrus | | | | | | | | |
| | sinensis | | | | | | | | |
| | GN = CISIN_1g0441782mg | | | | | | | | |
| | PE = 3 SV = 1 - | | | | | | | | |
| | [A0A067F810_CITSI] | | | | | | | | |
| A0A067G089 | Uncharacterized protein | 9.65 | 3 | 2 | 2 | 2 | 259 | 29.7 | 6.73 |
| | (Fragment) OS = Citrus | | | | | | | | |
| | sinensis | | | | | | | | |
| | GN = CISIN_1g044309mg | | | | | | | | |
| | PE = 4 SV = 1 - | | | | | | | | |
| | [A0A067G089_CITSI] | | | | | | | | |
| A0A067FTQ0 | Uncharacterized protein | 36.42 | 6 | 5 | 5 | 6 | 151 | 16.8 | 10.23 |
| | (Fragment) OS = Citrus | | | | | | | | |
| | sinensis | | | | | | | | |
| | GN = CISIN_1g044530mg | | | | | | | | |
| | PE = 3 SV = 1 - | | | | | | | | |
| | [A0A067FTQ0_CITSI] | | | | | | | | |
| A0A067FSA9 | Uncharacterized protein | 31.08 | 6 | 3 | 3 | 4 | 148 | 16.7 | 4.22 |
| | (Fragment) OS = Citrus | | | | | | | | |
| | sinensis | | | | | | | | |
| | GN = CISIN_1g0446441mg | | | | | | | | |
| | PE = 4 SV = 1 - | | | | | | | | |
| | [A0A067FSA9_CITSI] | | | | | | | | |
| A0A067EPH4 | Uncharacterized protein | 8.79 | 4 | 1 | 1 | 1 | 91 | 10.1 | 10.23 |
| | (Fragment) OS = Citrus | | | | | | | | |
| | sinensis | | | | | | | | |
| | GN = CISIN_1g044880mg | | | | | | | | |
| | PE = 3 SV = 1 - | | | | | | | | |
| | [A0A067EPH4_CITSI] | | | | | | | | |
| A0A067GPS1 | Uncharacterized protein | 10.95 | 3 | 3 | 3 | 3 | 283 | 32.4 | 4.79 |
| | (Fragment) OS = Citrus | | | | | | | | |
| | sinensis | | | | | | | | |
| | GN = CISIN_1g0453962mg | | | | | | | | |
| | PE = 3 SV = 1 - | | | | | | | | |
| | [A0A067GPS1_CITSI] | | | | | | | | |
| A0A067FP44 | Uncharacterized protein | 3.12 | 2 | 1 | 1 | 1 | 385 | 41.9 | 5.77 |
| | (Fragment) OS = Citrus | | | | | | | | |
| | sinensis | | | | | | | | |
| | GN = CISIN_1g0454311mg | | | | | | | | |
| | PE = 4 SV = 1 - | | | | | | | | |
| | [A0A067FP44_CITSI] | | | | | | | | |
| A0A067FC21 | Uncharacterized protein | 11.59 | 2 | 2 | 2 | 3 | 164 | 17.3 | 5.03 |
| | (Fragment) OS = Citrus | | | | | | | | |
| | sinensis | | | | | | | | |
| | GN = CISIN_1g045485mg | | | | | | | | |
| | PE = 4 SV = 1 - | | | | | | | | |
| | [A0A067FC21_CITSI] | | | | | | | | |
| A0A067G5W2 | Uncharacterized protein | 10.89 | 18 | 1 | 1 | 1 | 101 | 11.1 | 8.48 |
| | (Fragment) OS = Citrus | | | | | | | | |
| | sinensis | | | | | | | | |
| | GN = CISIN_1g045947mg | | | | | | | | |
| | PE = 3 SV = 1 - | | | | | | | | |
| | [A0A067G5W2_CITSI] | | | | | | | | |
| A0A067CZC4 | Uncharacterized protein | 13.67 | 4 | 2 | 2 | 2 | 256 | 28.4 | 6.01 |
| | (Fragment) OS = Citrus | | | | | | | | |
| | sinensis | | | | | | | | |
| | GN = CISIN_1g046608mg | | | | | | | | |
| | PE = 4 SV = 1 - | | | | | | | | |
| | [A0A067CZC4_CITSI] | | | | | | | | |
| A0A067F0F4 | Uncharacterized protein | 1.82 | 2 | 1 | 1 | 1 | 440 | 49.3 | 6.54 |
| | (Fragment) OS = Citrus | | | | | | | | |
| | sinensis | | | | | | | | |
| | GN = CISIN_1g0471651mg | | | | | | | | |
| | PE = 4 SV = 1 - | | | | | | | | |
| | [A0A067F0F4_CITSI] | | | | | | | | |
| A0A067D6B2 | Uncharacterized protein | 3.67 | 2 | 1 | 1 | 1 | 327 | 36.5 | 8.56 |
| | (Fragment) OS = Citrus | | | | | | | | |
| | sinensis | | | | | | | | |
| | GN = CISIN_1g047628mg | | | | | | | | |
| | PE = 4 SV = 1 - | | | | | | | | |
| | [A0A067D6B2_CITSI] | | | | | | | | |
| A0A067DTF1 | Uncharacterized protein | 3.93 | 3 | 1 | 1 | 2 | 229 | 25.2 | 7.66 |
| | (Fragment) OS = Citrus | | | | | | | | |
| | sinensis | | | | | | | | |
| | GN = CISIN_1g048775mg | | | | | | | | |
| | PE = 3 SV = 1 - | | | | | | | | |
| | [A0A067DTF1_CITSI] | | | | | | | | |
| V4T5E1 | Uncharacterized protein | 1.56 | 3 | 2 | 2 | 2 | 1088 | 123.0 | 6.07 |
| | OS = Citrus clementina | | | | | | | | |
| | GN = CICLE_v10000088mg | | | | | | | | |
| | PE = 3 SV = 1 - | | | | | | | | |
| | [V4T5E1_9ROSI] | | | | | | | | |
| V4TAL6 | Uncharacterized protein | 1.23 | 5 | 1 | 1 | 1 | 731 | 83.5 | 6.09 |
| | OS = Citrus clementina | | | | | | | | |
| | GN = CICLE_v10000256mg | | | | | | | | |
| | PE = 4 SV = 1 - | | | | | | | | |
| | [V4TAL6_9ROSI] | | | | | | | | |
| V4SY44 | Uncharacterized protein | 6.21 | 3 | 4 | 4 | 4 | 773 | 83.1 | 7.68 |
| | OS = Citrus clementina | | | | | | | | |
| | GN = CICLE_v10000355mg | | | | | | | | |
| | PE = 4 SV = 1 - | | | | | | | | |
| | [V4SY44_9ROSI] | | | | | | | | |
| V4SBF3 | Uncharacterized protein | 11.83 | 2 | 2 | 6 | 7 | 676 | 72.6 | 6.14 |
| | OS = Citrus clementina | | | | | | | | |
| | GN = CICLE_v10000502mg | | | | | | | | |
| | PE = 3 SV = 1 - | | | | | | | | |
| | [V4SBF3_9ROSI] | | | | | | | | |
| V4T5Q8 | Uncharacterized protein | 2.02 | 5 | 1 | 1 | 1 | 446 | 49.2 | 8.63 |
| | OS = Citrus clementina | | | | | | | | |
| | GN = CICLE_v10000783mg | | | | | | | | |
| | PE = 4 SV = 1 - | | | | | | | | |
| | [V4T5Q8_9ROSI] | | | | | | | | |
| V4T5E8 | Uncharacterized protein | 10.94 | 15 | 1 | 4 | 4 | 448 | 50.3 | 4.87 |
| | OS = Citrus clementina | | | | | | | | |
| | GN = CICLE_v10001140mg | | | | | | | | |
| | PE = 3 SV = 1 - | | | | | | | | |
| | [V4T5E8_9ROSI] | | | | | | | | |
| V4TAR1 | Uncharacterized protein | 6.38 | 3 | 2 | 2 | 2 | 423 | 47.4 | 5.11 |
| | OS = Citrus clementina | | | | | | | | |
| | GN = CICLE_v10001269mg | | | | | | | | |
| | PE = 3 SV = 1 - | | | | | | | | |
| | [V4TAR1_9ROSI] | | | | | | | | |
| V4SJ48 | Uncharacterized protein | 15.17 | 2 | 3 | 5 | 6 | 389 | 44.5 | 10.14 |
| | OS = Citrus clementina | | | | | | | | |
| | GN = CICLE_v10001454mg | | | | | | | | |
| | PE = 3 SV = 1 - | | | | | | | | |
| | [V4SJ48_9ROSI] | | | | | | | | |
| V4V353 | Uncharacterized protein | 2.72 | 4 | 1 | 1 | 1 | 294 | 31.7 | 5.54 |
| | OS = Citrus clementina | | | | | | | | |
| | GN = CICLE_v10001941mg | | | | | | | | |
| | PE = 4 SV = 1 - | | | | | | | | |
| | [V4V353_9ROSI] | | | | | | | | |
| V4V8J4 | Uncharacterized protein | 4.29 | 4 | 1 | 1 | 1 | 210 | 23.9 | 9.07 |
| | OS = Citrus clementina | | | | | | | | |
| | GN = CICLE_v10002227mg | | | | | | | | |
| | PE = 4 SV = 1 - | | | | | | | | |
| | [V4V8J4_9ROSI] | | | | | | | | |
| V4V566 | Uncharacterized protein | 13.49 | 1 | 1 | 3 | 5 | 252 | 28.5 | 4.87 |
| | OS = Citrus clementina | | | | | | | | |
| | GN = CICLE_v10002254mg | | | | | | | | |
| | PE = 3 SV = 1 - | | | | | | | | |
| | [V4V566_9ROSI] | | | | | | | | |
| V4SGH1 | Uncharacterized protein | 16.06 | 9 | 3 | 3 | 4 | 193 | 22.1 | 7.53 |
| | OS = Citrus clementina | | | | | | | | |
| | GN = CICLE_v10002566mg | | | | | | | | |
| | PE = 3 SV = 1 - | | | | | | | | |
| | [V4SGH1_9ROSI] | | | | | | | | |
| V4SGC9 | Uncharacterized protein | 11.23 | 3 | 1 | 2 | 2 | 187 | 21.0 | 11.05 |
| | OS = Citrus clementina | | | | | | | | |
| | GN = CICLE_v10002597mg | | | | | | | | |
| | PE = 4 SV = 1 - | | | | | | | | |
| | [V4SGC9_9ROSI] | | | | | | | | |
| V4T0Q3 | Uncharacterized protein | 13.10 | 3 | 1 | 2 | 3 | 168 | 19.0 | 4.55 |
| | OS = Citrus clementina | | | | | | | | |
| | GN = CICLE_v10002699mg | | | | | | | | |
| | PE = 4 SV = 1 - | | | | | | | | |
| | [V4T0Q3_9ROSI] | | | | | | | | |
| V4T5J0 | Uncharacterized protein | 17.09 | 3 | 2 | 2 | 2 | 158 | 17.8 | 4.70 |
| | OS = Citrus clementina | | | | | | | | |
| | GN = CICLE_v10002751mg | | | | | | | | |
| | PE = 4 SV = 1 - | | | | | | | | |
| | [V4T5J0_9ROSI] | | | | | | | | |
| V4V8U5 | Uncharacterized protein | 7.58 | 1 | 1 | 1 | 1 | 132 | 14.3 | 10.42 |
| | OS = Citrus clementina | | | | | | | | |
| | GN = CICLE_v10002830mg | | | | | | | | |
| | PE = 4 SV = 1 - | | | | | | | | |
| | [V4V8U5_9ROSI] | | | | | | | | |
| V4T1Q6 | Uncharacterized protein | 10.38 | 4 | 3 | 3 | 4 | 395 | 44.5 | 5.71 |
| | OS = Citrus clementina | | | | | | | | |
| | GN = CICLE_v10003767mg | | | | | | | | |
| | PE = 3 SV = 1 - | | | | | | | | |
| | [V4T1Q6_9ROSI] | | | | | | | | |
| V4U7H5 | Uncharacterized protein | 1.60 | 5 | 2 | 2 | 2 | 1312 | 144.1 | 5.85 |
| | OS = Citrus clementina | | | | | | | | |
| | GN = CICLE_v10004167mg | | | | | | | | |
| | PE = 4 SV = 1 - | | | | | | | | |
| | [V4U7H5_9ROSI] | | | | | | | | |
| V4RY27 | Uncharacterized protein | 11.45 | 6 | 4 | 4 | 5 | 428 | 45.8 | 6.21 |
| | OS = Citrus clementina | | | | | | | | |
| | GN = CICLE_v10004547mg | | | | | | | | |
| | PE = 4 SV = 1 - | | | | | | | | |
| | [V4RY27_9ROSI] | | | | | | | | |
| V4SDX2 | Uncharacterized protein | 2.00 | 2 | 1 | 1 | 1 | 601 | 64.3 | 5.58 |
| | OS = Citrus clementina | | | | | | | | |
| | GN = CICLE_v10004574mg | | | | | | | | |
| | PE = 4 SV = 1 - | | | | | | | | |
| | [V4SDX2_9ROSI] | | | | | | | | |
| V4RZ61 | Uncharacterized protein | 1.92 | 1 | 1 | 1 | 1 | 573 | 62.1 | 5.40 |
| | OS = Citrus clementina | | | | | | | | |
| | GN = CICLE_v10004635mg | | | | | | | | |
| | PE = 3 SV = 1 - | | | | | | | | |
| | [V4RZ61_9ROSI] | | | | | | | | |
| V4SEI1 | Uncharacterized protein | 3.80 | 2 | 2 | 2 | 2 | 553 | 61.6 | 6.70 |
| | OS = Citrus clementina | | | | | | | | |
| | GN = CICLE_v10004669mg | | | | | | | | |
| | PE = 3 SV = 1 - | | | | | | | | |
| | [V4SEI1_9ROSI] | | | | | | | | |
| V4S579 | Uncharacterized protein | 7.93 | 7 | 4 | 4 | 6 | 492 | 52.5 | 7.85 |
| | OS = Citrus clementina | | | | | | | | |
| | GN = CICLE_v10004713mg | | | | | | | | |
| | PE = 3 SV = 1 - | | | | | | | | |
| | [V4S579_9ROSI] | | | | | | | | |
| V4RJM1 | Uncharacterized protein | 5.13 | 2 | 2 | 2 | 3 | 487 | 53.9 | 6.35 |
| | OS = Citrus clementina | | | | | | | | |
| | GN = CICLE_v10004848mg | | | | | | | | |
| | PE = 4 SV = 1 - | | | | | | | | |
| | [V4RJM1_9ROSI] | | | | | | | | |
| V4RYA8 | Uncharacterized protein | 6.22 | 2 | 2 | 2 | 2 | 466 | 51.1 | 8.53 |
| | OS = Citrus clementina | | | | | | | | |
| | GN = CICLE_v10004918mg | | | | | | | | |
| | PE = 4 SV = 1 - | | | | | | | | |
| | [V4RYA8_9ROSI] | | | | | | | | |
| V4RJ07 | Uncharacterized protein | 7.37 | 3 | 2 | 2 | 3 | 339 | 38.2 | 8.03 |
| | OS = Citrus clementina | | | | | | | | |
| | GN = CICLE_v10005363mg | | | | | | | | |
| | PE = 4 SV = 1 - | | | | | | | | |
| | [V4RJ07_9ROSI] | | | | | | | | |
| V4S211 | Uncharacterized protein | 4.57 | 3 | 1 | 1 | 1 | 219 | 24.9 | 4.73 |
| | OS = Citrus clementina | | | | | | | | |
| | GN = CICLE_v10005378mg | | | | | | | | |
| | PE = 4 SV = 1 - | | | | | | | | |
| | [V4S211_9ROSI] | | | | | | | | |
| V4SA34 | Uncharacterized protein | 4.57 | 2 | 2 | 2 | 2 | 328 | 36.6 | 7.40 |
| | OS = Citrus clementina | | | | | | | | |
| | GN = CICLE_v10005408mg | | | | | | | | |
| | PE = 4 SV = 1 - | | | | | | | | |
| | [V4SA34_9ROSI] | | | | | | | | |
| V4S879 | Uncharacterized protein | 3.77 | 4 | 1 | 1 | 1 | 239 | 25.6 | 6.90 |
| | OS = Citrus clementina | | | | | | | | |
| | GN = CICLE_v10005448mg | | | | | | | | |
| | PE = 4 SV = 1 - | | | | | | | | |
| | [V4S879_9ROSI] | | | | | | | | |
| V4S8P5 | Uncharacterized protein | 7.51 | 1 | 2 | 2 | 2 | 253 | 27.9 | 9.17 |
| | OS = Citrus clementina | | | | | | | | |
| | GN = CICLE_v10005703mg | | | | | | | | |
| | PE = 4 SV = 1 - | | | | | | | | |
| | [V4S8P5_9ROSI] | | | | | | | | |
| V4U096 | Uncharacterized protein | 8.61 | 3 | 1 | 1 | 7 | 151 | 16.0 | 6.15 |
| | OS = Citrus clementina | | | | | | | | |
| | GN = CICLE_v10006030mg | | | | | | | | |
| | PE = 4 SV = 1 - | | | | | | | | |
| | [V4U096_9ROSI] | | | | | | | | |
| V4U7Q5 | Uncharacterized protein | 5.36 | 1 | 1 | 1 | 1 | 168 | 19.2 | 4.94 |
| | OS = Citrus clementina | | | | | | | | |
| | GN = CICLE_v10006072mg | | | | | | | | |
| | PE = 4 SV = 1 - | | | | | | | | |
| | [V4U7Q5_9ROSI] | | | | | | | | |
| V4S3U3 | Uncharacterized protein | 20.71 | 3 | 2 | 2 | 2 | 140 | 16.0 | 10.54 |
| | OS = Citrus clementina | | | | | | | | |
| | GN = CICLE_v10006145mg | | | | | | | | |
| | PE = 3 SV = 1 - | | | | | | | | |
| | [V4S3U3_9ROSI] | | | | | | | | |
| V4S036 | Uncharacterized protein | 12.03 | 2 | 2 | 2 | 4 | 241 | 27.4 | 6.32 |
| | OS = Citrus clementina | | | | | | | | |
| | GN = CICLE_v10006723mg | | | | | | | | |
| | PE = 4 SV = 1 - | | | | | | | | |
| | [V4S036_9ROSI] | | | | | | | | |
| V4S0M9 | Uncharacterized protein | 19.53 | 9 | 3 | 3 | 3 | 128 | 14.7 | 9.92 |
| | OS = Citrus clementina | | | | | | | | |
| | GN = CICLE_v10006876mg | | | | | | | | |
| | PE = 4 SV = 1 - | | | | | | | | |
| | [V4S0M9_9ROSI] | | | | | | | | |
| V4U0G6 | Uncharacterized protein | 6.19 | 7 | 4 | 4 | 5 | 776 | 85.7 | 5.97 |
| | OS = Citrus clementina | | | | | | | | |
| | GN = CICLE_v10007519mg | | | | | | | | |
| | PE = 4 SV = 1 - | | | | | | | | |
| | [V4U0G6_9ROSI] | | | | | | | | |
| V4TU44 | Uncharacterized protein | 1.35 | 1 | 1 | 1 | 1 | 669 | 69.2 | 5.97 |
| | OS = Citrus clementina | | | | | | | | |
| | GN = CICLE_v10007663mg | | | | | | | | |
| | PE = 4 SV = 1 - | | | | | | | | |
| | [V4TU44_9ROSI] | | | | | | | | |
| V4UR81 | Uncharacterized protein | 2.35 | 1 | 1 | 1 | 1 | 637 | 73.4 | 7.59 |
| | OS = Citrus clementina | | | | | | | | |
| | GN = CICLE_v10007727mg | | | | | | | | |
| | PE = 4 SV = 1 - | | | | | | | | |
| | [V4UR81_9ROSI] | | | | | | | | |
| V4WJB1 | Uncharacterized protein | 5.66 | 1 | 2 | 2 | 3 | 583 | 60.8 | 7.31 |
| | OS = Citrus clementina | | | | | | | | |
| | GN = CICLE_v10007834mg | | | | | | | | |
| | PE = 3 SV = 1 - | | | | | | | | |
| | [V4WJB1_9ROSI] | | | | | | | | |
| V4U0R4 | Uncharacterized protein | 5.59 | 5 | 2 | 2 | 2 | 537 | 58.3 | 7.47 |
| | OS = Citrus clementina | | | | | | | | |
| | GN = CICLE_v10007954mg | | | | | | | | |
| | PE = 4 SV = 1 - | | | | | | | | |
| | [V4U0R4_9ROSI] | | | | | | | | |
| V4UCF3 | Uncharacterized protein | 3.79 | 5 | 1 | 1 | 1 | 343 | 38.9 | 5.41 |
| | OS = Citrus clementina | | | | | | | | |
| | GN = CICLE_v10008383mg | | | | | | | | |
| | PE = 4 SV = 1 - | | | | | | | | |
| | [V4UCF3_9ROSI] | | | | | | | | |
| V4UV09 | Uncharacterized protein | 11.85 | 1 | 4 | 4 | 4 | 422 | 47.0 | 6.11 |
| | OS = Citrus clementina | | | | | | | | |
| | GN = CICLE_v10008409mg | | | | | | | | |
| | PE = 4 SV = 1 - | | | | | | | | |
| | [V4UV09_9ROSI] | | | | | | | | |
| V4USP2 | Uncharacterized protein | 7.50 | 6 | 2 | 2 | 2 | 320 | 35.5 | 6.14 |
| | OS = Citrus clementina | | | | | | | | |
| | GN = CICLE_v10008440mg | | | | | | | | |
| | PE = 4 SV = 1 - | | | | | | | | |
| | [V4USP2_9ROSI] | | | | | | | | |
| V4UJI5 | Uncharacterized protein | 23.30 | 7 | 1 | 9 | 10 | 412 | 46.7 | 5.68 |
| | OS = Citrus clementina | | | | | | | | |
| | GN = CICLE_v10008468mg | | | | | | | | |
| | PE = 3 SV = 1 - | | | | | | | | |
| | [V4UJI5_9ROSI] | | | | | | | | |
| V4TVZ8 | Uncharacterized protein | 5.04 | 6 | 1 | 1 | 1 | 278 | 32.2 | 4.28 |
| | OS = Citrus clementina | | | | | | | | |
| | GN = CICLE_v10008686mg | | | | | | | | |
| | PE = 3 SV = 1 - | | | | | | | | |
| | [V4TVZ8_9ROSI] | | | | | | | | |
| V4UJW6 | Uncharacterized protein | 9.97 | 2 | 3 | 3 | 3 | 351 | 36.7 | 9.04 |
| | OS = Citrus clementina | | | | | | | | |
| | GN = CICLE_v10008786mg | | | | | | | | |
| | PE = 4 SV = 1 - | | | | | | | | |
| | [V4UJW6_9ROSI] | | | | | | | | |
| V4TWR9 | Uncharacterized protein | 15.42 | 11 | 3 | 3 | 3 | 201 | 21.9 | 5.87 |
| | OS = Citrus clementina | | | | | | | | |
| | GN = CICLE_v10009133mg | | | | | | | | |
| | PE = 4 SV = 1 - | | | | | | | | |
| | [V4TWR9_9ROSI] | | | | | | | | |
| V4TY99 | Uncharacterized protein | 16.46 | 5 | 2 | 2 | 3 | 158 | 16.7 | 8.57 |
| | OS = Citrus clementina | | | | | | | | |
| | GN = CICLE_v10009443mg | | | | | | | | |
| | PE = 4 SV = 1 - | | | | | | | | |
| | [V4TY99_9ROSI] | | | | | | | | |
| V4UK18 | Uncharacterized protein | 5.00 | 2 | 1 | 1 | 1 | 200 | 22.4 | 4.77 |
| | OS = Citrus clementina | | | | | | | | |
| | GN = CICLE_v10009534mg | | | | | | | | |
| | PE = 4 SV = 1 - | | | | | | | | |
| | [V4UK18_9ROSI] | | | | | | | | |
| V4WED4 | Uncharacterized protein | 6.40 | 1 | 1 | 1 | 2 | 172 | 18.9 | 7.44 |
| | OS = Citrus clementina | | | | | | | | |
| | GN = CICLE_v10009697mg | | | | | | | | |
| | PE = 4 SV = 1 - | | | | | | | | |
| | [V4WED4_9ROSI] | | | | | | | | |
| V4UJF9 | Uncharacterized protein | 20.63 | 2 | 3 | 3 | 6 | 160 | 17.8 | 5.73 |
| | OS = Citrus clementina | | | | | | | | |
| | GN = CICLE_v10009756mg | | | | | | | | |
| | PE = 3 SV = 1 - | | | | | | | | |
| | [V4UJF9_9ROSI] | | | | | | | | |
| V4TVN3 | Uncharacterized protein | 19.33 | 1 | 2 | 2 | 3 | 150 | 16.7 | 10.90 |
| | OS = Citrus clementina | | | | | | | | |
| | GN = CICLE_v10009798mg | | | | | | | | |
| | PE = 4 SV = 1 - | | | | | | | | |
| | [V4TVN3_9ROSI] | | | | | | | | |
| V4TSF8 | Uncharacterized protein | 8.08 | 1 | 1 | 1 | 1 | 99 | 10.8 | 6.74 |
| | OS = Citrus clementina | | | | | | | | |
| | GN = CICLE_v10010015mg | | | | | | | | |
| | PE = 4 SV = 1 - | | | | | | | | |
| | [V4TSF8_9ROSI] | | | | | | | | |
| V4T4K0 | Uncharacterized protein | 4.21 | 5 | 2 | 3 | 3 | 973 | 109.4 | 6.46 |
| | OS = Citrus clementina | | | | | | | | |
| | GN = CICLE_v10010991mg | | | | | | | | |
| | PE = 3 SV = 1 - | | | | | | | | |
| | [V4T4K0_9ROSI] | | | | | | | | |
| V4SRW7 | Uncharacterized protein | 16.78 | 8 | 2 | 13 | 14 | 864 | 96.1 | 5.35 |
| | OS = Citrus clementina | | | | | | | | |
| | GN = CICLE_v10011046mg | | | | | | | | |
| | PE = 3 SV = 1 - | | | | | | | | |
| | [V4SRW7_9ROSI] | | | | | | | | |
| V4S4E8 | Uncharacterized protein | 28.59 | 6 | 2 | 13 | 33 | 647 | 70.8 | 5.29 |
| | OS = Citrus clementina | | | | | | | | |
| | GN = CICLE_v10011260mg | | | | | | | | |
| | PE = 3 SV = 1 - | | | | | | | | |
| | [V4S4E8_9ROSI] | | | | | | | | |
| V4SZN9 | Uncharacterized protein | 2.25 | 3 | 1 | 1 | 1 | 488 | 52.1 | 6.46 |
| | OS = Citrus clementina | | | | | | | | |
| | GN = CICLE_v10011317mg | | | | | | | | |
| | PE = 3 SV = 1 - | | | | | | | | |
| | [V4SZN9_9ROSI] | | | | | | | | |
| V4SY02 | Uncharacterized protein | 4.24 | 5 | 2 | 2 | 3 | 472 | 50.3 | 6.73 |
| | OS = Citrus clementina | | | | | | | | |
| | GN = CICLE_v10011371mg | | | | | | | | |
| | PE = 3 SV = 1 - | | | | | | | | |
| | [V4SY02_9ROSI] | | | | | | | | |
| V4SY74 | Uncharacterized protein | 12.34 | 4 | 6 | 6 | 6 | 535 | 58.9 | 6.49 |
| | OS = Citrus clementina | | | | | | | | |
| | GN = CICLE_v10011450mg | | | | | | | | |
| | PE = 3 SV = 1 - | | | | | | | | |
| | [V4SY74_9ROSI] | | | | | | | | |
| V4SSI3 | Uncharacterized protein | 28.34 | 5 | 10 | 10 | 15 | 441 | 48.8 | 6.06 |
| | OS = Citrus clementina | | | | | | | | |
| | GN = CICLE_v10011657mg | | | | | | | | |
| | PE = 4 SV = 1 - | | | | | | | | |
| | [V4SSI3_9ROSI] | | | | | | | | |
| V4SY92 | Uncharacterized protein | 15.31 | 4 | 6 | 6 | 9 | 405 | 44.6 | 10.37 |
| | OS = Citrus clementina | | | | | | | | |
| | GN = CICLE_v10011878mg | | | | | | | | |
| | PE = 4 SV = 1 - | | | | | | | | |
| | [V4SY92_9ROSI] | | | | | | | | |
| V4UQK8 | Uncharacterized protein | 2.30 | 2 | 1 | 1 | 1 | 392 | 43.3 | 4.44 |
| | OS = Citrus clementina | | | | | | | | |
| | GN = CICLE_v10011923mg | | | | | | | | |
| | PE = 4 SV = 1 - | | | | | | | | |
| | [V4UQK8_9ROSI] | | | | | | | | |
| V4T3H6 | Uncharacterized protein | 6.82 | 5 | 1 | 1 | 1 | 220 | 23.7 | 9.57 |
| | OS = Citrus clementina | | | | | | | | |
| | GN = CICLE_v10012367mg | | | | | | | | |
| | PE = 4 SV = 1 - | | | | | | | | |
| | [V4T3H6_9ROSI] | | | | | | | | |
| V4UWC9 | Uncharacterized protein | 52.09 | 19 | 7 | 10 | 14 | 263 | 29.7 | 4.81 |
| | OS = Citrus clementina | | | | | | | | |
| | GN = CICLE_v10012495mg | | | | | | | | |
| | PE = 3 SV = 1 - | | | | | | | | |
| | [V4UWC9_9ROSI] | | | | | | | | |
| V4SSK7 | Uncharacterized protein | 17.82 | 3 | 4 | 4 | 4 | 202 | 22.5 | 6.79 |
| | OS = Citrus clementina | | | | | | | | |
| | GN = CICLE_v10012559mg | | | | | | | | |
| | PE = 4 SV = 1 - | | | | | | | | |
| | [V4SSK7_9ROSI] | | | | | | | | |
| V4URM7 | Uncharacterized protein | 8.47 | 7 | 2 | 2 | 3 | 236 | 27.1 | 5.58 |
| | OS = Citrus clementina | | | | | | | | |
| | GN = CICLE_v10012631mg | | | | | | | | |
| | PE = 4 SV = 1 - | | | | | | | | |
| | [V4URM7_9ROSI] | | | | | | | | |
| V4T409 | Uncharacterized protein | 3.88 | 1 | 1 | 1 | 1 | 206 | 21.8 | 9.52 |
| | OS = Citrus clementina | | | | | | | | |
| | GN = CICLE_v10012786mg | | | | | | | | |
| | PE = 4 SV = 1 - | | | | | | | | |
| | [V4T409_9ROSI] | | | | | | | | |
| V4STZ5 | Uncharacterized protein | 5.49 | 1 | 1 | 1 | 2 | 164 | 18.2 | 6.34 |
| | OS = Citrus clementina | | | | | | | | |
| | GN = CICLE_v10012955mg | | | | | | | | |
| | PE = 4 SV = 1 - | | | | | | | | |
| | [V4STZ5_9ROSI] | | | | | | | | |
| V4S7V0 | Uncharacterized protein | 5.26 | 1 | 1 | 1 | 1 | 152 | 16.7 | 4.97 |
| | OS = Citrus clementina | | | | | | | | |
| | GN = CICLE_v10013017mg | | | | | | | | |
| | PE = 4 SV = 1 - | | | | | | | | |
| | [V4S7V0_9ROSI] | | | | | | | | |
| V4SP59 | Uncharacterized protein | 26.21 | 6 | 1 | 4 | 10 | 145 | 16.3 | 8.35 |
| | OS = Citrus clementina | | | | | | | | |
| | GN = CICLE_v10013046mg | | | | | | | | |
| | PE = 3 SV = 1 - | | | | | | | | |
| | [V4SP59_9ROSI] | | | | | | | | |
| V4SUL0 | Uncharacterized protein | 26.60 | 3 | 2 | 2 | 17 | 94 | 10.3 | 6.15 |
| | OS = Citrus clementina | | | | | | | | |
| | GN = CICLE_v10013203mg | | | | | | | | |
| | PE = 4 SV = 1 - | | | | | | | | |
| | [V4SUL0_9ROSI] | | | | | | | | |
| V4ULQ3 | Uncharacterized protein | 7.64 | 1 | 1 | 1 | 1 | 144 | 15.7 | 9.66 |
| | OS = Citrus clementina | | | | | | | | |
| | GN = CICLE_v10013953mg | | | | | | | | |
| | PE = 4 SV = 1 - | | | | | | | | |
| | [V4ULQ3_9ROSI] | | | | | | | | |
| V4UC83 | Uncharacterized protein | 1.10 | 1 | 1 | 1 | 1 | 913 | 100.8 | 4.87 |
| | OS = Citrus clementina | | | | | | | | |
| | GN = CICLE_v10014189mg | | | | | | | | |
| | PE = 4 SV = 1 - | | | | | | | | |
| | [V4UC83_9ROSI] | | | | | | | | |
| V4W5U2 | Uncharacterized protein | 21.78 | 5 | 10 | 10 | 11 | 583 | 63.3 | 5.73 |
| | OS = Citrus clementina | | | | | | | | |
| | GN = CICLE_v10014721mg | | | | | | | | |
| | PE = 3 SV = 1 - | | | | | | | | |
| | [V4W5U2_9ROSI] | | | | | | | | |
| V4U5S2 | Uncharacterized protein | 4.69 | 4 | 2 | 2 | 2 | 469 | 51.7 | 6.18 |
| | OS = Citrus clementina | | | | | | | | |
| | GN = CICLE_v10015044mg | | | | | | | | |
| | PE = 4 SV = 1 - | | | | | | | | |
| | [V4U5S2_9ROSI] | | | | | | | | |
| V4UJ28 | Uncharacterized protein | 5.28 | 6 | 2 | 2 | 2 | 322 | 35.8 | 9.47 |
| | OS = Citrus clementina | | | | | | | | |
| | GN = CICLE_v10015210mg | | | | | | | | |
| | PE = 4 SV = 1 - | | | | | | | | |
| | [V4UJ28_9ROSI] | | | | | | | | |
| V4W4S7 | Uncharacterized protein | 5.68 | 3 | 2 | 2 | 3 | 405 | 41.4 | 6.87 |
| | OS = Citrus clementina | | | | | | | | |
| | GN = CICLE_v10015452mg | | | | | | | | |
| | PE = 3 SV = 1 - | | | | | | | | |
| | [V4W4S7_9ROSI] | | | | | | | | |
| V4U873 | Uncharacterized protein | 4.13 | 3 | 1 | 1 | 1 | 387 | 42.8 | 7.17 |
| | OS = Citrus clementina | | | | | | | | |
| | GN = CICLE_v10015526mg | | | | | | | | |
| | PE = 4 SV = 1 - | | | | | | | | |
| | [V4U873_9ROSI] | | | | | | | | |
| V4UJI6 | Uncharacterized protein | 12.00 | 5 | 3 | 3 | 3 | 325 | 34.5 | 6.98 |
| | OS = Citrus clementina | | | | | | | | |
| | GN = CICLE_v10015925mg | | | | | | | | |
| | PE = 4 SV = 1 - | | | | | | | | |
| | [V4UJI6_9ROSI] | | | | | | | | |
| V4W9L3 | Uncharacterized protein | 16.84 | 12 | 4 | 4 | 4 | 291 | 32.6 | 5.59 |
| | OS = Citrus clementina | | | | | | | | |
| | GN = CICLE_v10016136mg | | | | | | | | |
| | PE = 4 SV = 1 - | | | | | | | | |
| | [V4W9L3_9ROSI] | | | | | | | | |
| V4VZP8 | Uncharacterized protein | 3.82 | 2 | 1 | 1 | 1 | 288 | 32.6 | 7.15 |
| | OS = Citrus clementina | | | | | | | | |
| | GN = CICLE_v10016149mg | | | | | | | | |
| | PE = 4 SV = 1 - | | | | | | | | |
| | [V4VZP8_9ROSI] | | | | | | | | |
| V4U779 | Uncharacterized protein | 10.88 | 3 | 2 | 2 | 2 | 239 | 26.7 | 8.87 |
| | OS = Citrus clementina | | | | | | | | |
| | GN = CICLE_v10016485mg | | | | | | | | |
| | PE = 4 SV = 1 - | | | | | | | | |
| | [V4U779_9ROSI] | | | | | | | | |
| V4UBI0 | Uncharacterized protein | 18.43 | 7 | 2 | 3 | 6 | 217 | 23.4 | 5.05 |
| | OS = Citrus clementina | | | | | | | | |
| | GN = CICLE_v10016592mg | | | | | | | | |
| | PE = 3 SV = 1 - | | | | | | | | |
| | [V4UBI0_9ROSI] | | | | | | | | |
| V4UCQ9 | Uncharacterized protein | 10.78 | 4 | 2 | 2 | 2 | 167 | 19.4 | 9.79 |
| | OS = Citrus clementina | | | | | | | | |
| | GN = CICLE_v10016875mg | | | | | | | | |
| | PE = 3 SV = 1 - | | | | | | | | |
| | [V4UCQ9_9ROSI] | | | | | | | | |
| V4U8T6 | Uncharacterized protein | 7.09 | 2 | 1 | 1 | 1 | 127 | 14.1 | 4.82 |
| | OS = Citrus clementina | | | | | | | | |
| | GN = CICLE_v10017174mg | | | | | | | | |
| | PE = 4 SV = 1 - | | | | | | | | |
| | [V4U8T6_9ROSI] | | | | | | | | |
| V4TZA6 | Uncharacterized protein | 11.11 | 1 | 1 | 1 | 1 | 81 | 9.5 | 10.05 |
| | OS = Citrus clementina | | | | | | | | |
| | GN = CICLE_v10017697mg | | | | | | | | |
| | PE = 4 SV = 1 - | | | | | | | | |
| | [V4TZA6_9ROSI] | | | | | | | | |
| V4UAW2 | Uncharacterized protein | 16.25 | 1 | 1 | 1 | 1 | 80 | 8.9 | 8.73 |
| | OS = Citrus clementina | | | | | | | | |
| | GN = CICLE_v10018398mg | | | | | | | | |
| | PE = 4 SV = 1 - | | | | | | | | |
| | [V4UAW2_9ROSI] | | | | | | | | |
| V4T3X0 | Uncharacterized protein | 4.39 | 5 | 4 | 4 | 4 | 1024 | 116.0 | 6.98 |
| | OS = Citrus clementina | | | | | | | | |
| | GN = CICLE_v10018656mg | | | | | | | | |
| | PE = 4 SV = 1 - | | | | | | | | |
| | [V4T3X0_9ROSI] | | | | | | | | |
| V4VXE2 | Uncharacterized protein | 0.92 | 1 | 1 | 1 | 1 | 975 | 109.5 | 5.82 |
| | OS = Citrus clementina | | | | | | | | |
| | GN = CICLE_v10018709mg | | | | | | | | |
| | PE = 4 SV = 1 - | | | | | | | | |
| | [V4VXE2_9ROSI] | | | | | | | | |
| V4TS31 | Uncharacterized protein | 2.88 | 36 | 2 | 2 | 2 | 834 | 92.6 | 6.98 |
| | OS = Citrus clementina | | | | | | | | |
| | GN = CICLE_v10018858mg | | | | | | | | |
| | PE = 3 SV = 1 - | | | | | | | | |
| | [V4TS31_9ROSI] | | | | | | | | |
| V4VU32 | Uncharacterized protein | 7.19 | 14 | 5 | 5 | 7 | 765 | 84.6 | 6.93 |
| | OS = Citrus clementina | | | | | | | | |
| | GN = CICLE_v10018965mg | | | | | | | | |
| | PE = 3 SV = 1 - | | | | | | | | |
| | [V4VU32_9ROSI] | | | | | | | | |
| V4TSH7 | Uncharacterized protein | 5.54 | 4 | 3 | 3 | 3 | 740 | 83.1 | 8.85 |
| | OS = Citrus clementina | | | | | | | | |
| | GN = CICLE_v10019005mg | | | | | | | | |
| | PE = 4 SV = 1 - | | | | | | | | |
| | [V4TSH7_9ROSI] | | | | | | | | |
| V4SX86 | Uncharacterized protein | 12.44 | 7 | 4 | 7 | 12 | 667 | 73.5 | 5.21 |
| | OS = Citrus clementina | | | | | | | | |
| | GN = CICLE_v10019184mg | | | | | | | | |
| | PE = 3 SV = 1 - | | | | | | | | |
| | [V4SX86_9ROSI] | | | | | | | | |
| V4T007 | Uncharacterized protein | 2.42 | 2 | 1 | 1 | 1 | 414 | 45.0 | 9.16 |
| | OS = Citrus clementina | | | | | | | | |
| | GN = CICLE_v10019636mg | | | | | | | | |
| | PE = 3 SV = 1 - | | | | | | | | |
| | [V4T007_9ROSI] | | | | | | | | |
| V4VJT4 | Uncharacterized protein | 4.19 | 4 | 2 | 2 | 2 | 501 | 54.5 | 5.52 |
| | OS = Citrus clementina | | | | | | | | |
| | GN = CICLE_v10019800mg | | | | | | | | |
| | PE = 3 SV = 1 - | | | | | | | | |
| | [V4VJT4_9ROSI] | | | | | | | | |
| V4SXA7 | Uncharacterized protein | 5.87 | 2 | 1 | 1 | 1 | 494 | 52.5 | 8.00 |
| | OS = Citrus clementina | | | | | | | | |
| | GN = CICLE_v10019855mg | | | | | | | | |
| | PE = 3 SV = 1 - | | | | | | | | |
| | [V4SXA7_9ROSI] | | | | | | | | |
| V4TN38 | Uncharacterized protein | 5.69 | 8 | 1 | 2 | 4 | 422 | 48.0 | 6.18 |
| | OS = Citrus clementina | | | | | | | | |
| | GN = CICLE_v10020303mg | | | | | | | | |
| | PE = 4 SV = 1 - | | | | | | | | |
| | [V4TN38_9ROSI] | | | | | | | | |
| V4VRW5 | Uncharacterized protein | 12.85 | 10 | 4 | 4 | 4 | 358 | 38.2 | 6.01 |
| | OS = Citrus clementina | | | | | | | | |
| | GN = CICLE_v10020809mg | | | | | | | | |
| | PE = 4 SV = 1 - | | | | | | | | |
| | [V4VRW5_9ROSI] | | | | | | | | |
| V4VP55 | Uncharacterized protein | 11.03 | 6 | 2 | 2 | 2 | 281 | 29.3 | 8.60 |
| | OS = Citrus clementina | | | | | | | | |
| | GN = CICLE_v10020931mg | | | | | | | | |
| | PE = 3 SV = 1 - | | | | | | | | |
| | [V4VP55_9ROSI] | | | | | | | | |
| V4U283 | Uncharacterized protein | 20.36 | 3 | 5 | 5 | 6 | 329 | 35.1 | 5.11 |
| | OS = Citrus clementina | | | | | | | | |
| | GN = CICLE_v10021105mg | | | | | | | | |
| | PE = 3 SV = 1 - | | | | | | | | |
| | [V4U283_9ROSI] | | | | | | | | |
| V4TRS7 | Uncharacterized protein | 5.57 | 1 | 2 | 2 | 15 | 323 | 35.5 | 7.09 |
| | OS = Citrus clementina | | | | | | | | |
| | GN = CICLE_v10021162mg | | | | | | | | |
| | PE = 4 SV = 1 - | | | | | | | | |
| | [V4TRS7_9ROSI] | | | | | | | | |
| V4TBH2 | Uncharacterized protein | 5.78 | 4 | 1 | 1 | 1 | 173 | 19.6 | 5.38 |
| | OS = Citrus clementina | | | | | | | | |
| | GN = CICLE_v10021228mg | | | | | | | | |
| | PE = 4 SV = 1 - | | | | | | | | |
| | [V4TBH2_9ROSI] | | | | | | | | |
| V4TNR7 | Uncharacterized protein | 9.39 | 3 | 1 | 1 | 1 | 181 | 19.9 | 7.49 |
| | OS = Citrus clementina | | | | | | | | |
| | GN = CICLE_v10021896mg | | | | | | | | |
| | PE = 3 SV = 1 - | | | | | | | | |
| | [V4TNR7_9ROSI] | | | | | | | | |
| V4TX11 | Uncharacterized protein | 3.43 | 1 | 1 | 1 | 1 | 233 | 25.6 | 7.96 |
| | OS = Citrus clementina | | | | | | | | |
| | GN = CICLE_v10022024mg | | | | | | | | |
| | PE = 3 SV = 1 - | | | | | | | | |
| | [V4TX11_9ROSI] | | | | | | | | |
| V4VLZ2 | Uncharacterized protein | 7.11 | 1 | 1 | 1 | 1 | 225 | 24.7 | 7.94 |
| | OS = Citrus clementina | | | | | | | | |
| | GN = CICLE_v10022104mg | | | | | | | | |
| | PE = 4 SV = 1 - | | | | | | | | |
| | [V4VLZ2_9ROSI] | | | | | | | | |
| V4TB09 | Uncharacterized protein | 8.45 | 1 | 1 | 1 | 2 | 213 | 23.8 | 6.64 |
| | OS = Citrus clementina | | | | | | | | |
| | GN = CICLE_v10022212mg | | | | | | | | |
| | PE = 3 SV = 1 - | | | | | | | | |
| | [V4TB09_9ROSI] | | | | | | | | |
| V4VJ85 | Uncharacterized protein | 19.89 | 4 | 3 | 3 | 3 | 181 | 19.8 | 9.67 |
| | OS = Citrus clementina | | | | | | | | |
| | GN = CICLE_v10022481mg | | | | | | | | |
| | PE = 4 SV = 1 - | | | | | | | | |
| | [V4VJ85_9ROSI] | | | | | | | | |
| V4TVM3 | Uncharacterized protein | 36.28 | 17 | 3 | 3 | 3 | 113 | 13.0 | 8.84 |
| | OS = Citrus clementina | | | | | | | | |
| | GN = CICLE_v10022662mg | | | | | | | | |
| | PE = 3 SV = 1 - | | | | | | | | |
| | [V4TVM3_9ROSI] | | | | | | | | |
| V4U4X4 | Uncharacterized protein | 8.05 | 1 | 1 | 1 | 2 | 149 | 17.0 | 4.22 |
| | OS = Citrus clementina | | | | | | | | |
| | GN = CICLE_v10022698mg | | | | | | | | |
| | PE = 4 SV = 1 - | | | | | | | | |
| | [V4U4X4_9ROSI] | | | | | | | | |
| V4U5Y8 | Uncharacterized protein | 16.80 | 1 | 1 | 2 | 2 | 125 | 14.0 | 9.55 |
| | OS = Citrus clementina | | | | | | | | |
| | GN = CICLE_v10022870mg | | | | | | | | |
| | PE = 4 SV = 1 - | | | | | | | | |
| | [V4U5Y8_9ROSI] | | | | | | | | |
| V4VP83 | Uncharacterized protein | 9.18 | 1 | 1 | 1 | 1 | 98 | 11.0 | 10.35 |
| | OS = Citrus clementina | | | | | | | | |
| | GN = CICLE_v10023033mg | | | | | | | | |
| | PE = 4 SV = 1 - | | | | | | | | |
| | [V4VP83_9ROSI] | | | | | | | | |
| V4TVF9 | Uncharacterized protein | 13.48 | 2 | 1 | 1 | 1 | 89 | 9.7 | 4.41 |
| | OS = Citrus clementina | | | | | | | | |
| | GN = CICLE_v10023083mg | | | | | | | | |
| | PE = 4 SV = 1 - | | | | | | | | |
| | [V4TVF9_9ROSI] | | | | | | | | |
| V4VS24 | Uncharacterized protein | 12.99 | 7 | 1 | 1 | 1 | 77 | 9.0 | 10.42 |
| | OS = Citrus clementina | | | | | | | | |
| | GN = CICLE_v10023654mg | | | | | | | | |
| | PE = 4 SV = 1 - | | | | | | | | |
| | [V4VS24_9ROSI] | | | | | | | | |
| V4U076 | Uncharacterized protein | 8.92 | 2 | 1 | 1 | 2 | 157 | 17.9 | 4.77 |
| | OS = Citrus clementina | | | | | | | | |
| | GN = CICLE_v10024324mg | | | | | | | | |
| | PE = 4 SV = 1 - | | | | | | | | |
| | [V4U076_9ROSI] | | | | | | | | |
| V4SF31 | Uncharacterized protein | 1.25 | 1 | 1 | 1 | 1 | 560 | 62.6 | 6.61 |
| | OS = Citrus clementina | | | | | | | | |
| | GN = CICLE_v10025298mg | | | | | | | | |
| | PE = 4 SV = 1 - | | | | | | | | |
| | [V4SF31_9ROSI] | | | | | | | | |
| V4SHF3 | Uncharacterized protein | 1.89 | 3 | 1 | 1 | 1 | 529 | 58.0 | 6.74 |
| | OS = Citrus clementina | | | | | | | | |
| | GN = CICLE_v10025334mg | | | | | | | | |
| | PE = 4 SV = 1 - | | | | | | | | |
| | [V4SHF3_9ROSI] | | | | | | | | |
| V4UK96 | Uncharacterized protein | 10.12 | 15 | 2 | 2 | 2 | 326 | 35.8 | 6.14 |
| | OS = Citrus clementina | | | | | | | | |
| | GN = CICLE_v10025540mg | | | | | | | | |
| | PE = 4 SV = 1 - | | | | | | | | |
| | [V4UK96_9ROSI] | | | | | | | | |
| V4SN96 | Uncharacterized protein | 8.40 | 9 | 1 | 1 | 1 | 131 | 13.9 | 6.51 |
| | OS = Citrus clementina | | | | | | | | |
| | GN = CICLE_v10026577mg | | | | | | | | |
| | PE = 4 SV = 1 - | | | | | | | | |
| | [V4SN96_9ROSI] | | | | | | | | |
| V4SQH9 | Uncharacterized protein | 4.79 | 1 | 1 | 1 | 2 | 146 | 16.8 | 10.90 |
| | OS = Citrus clementina | | | | | | | | |
| | GN = CICLE_v10026719mg | | | | | | | | |
| | PE = 3 SV = 1 - | | | | | | | | |
| | [V4SQH9_9ROSI] | | | | | | | | |
| V4SJ98 | Uncharacterized protein | 6.72 | 1 | 1 | 1 | 1 | 134 | 14.8 | 7.05 |
| | OS = Citrus clementina | | | | | | | | |
| | GN = CICLE_v10026748mg | | | | | | | | |
| | PE = 4 SV = 1 - | | | | | | | | |
| | [V4SJ98_9ROSI] | | | | | | | | |
| V4RNH3 | Uncharacterized protein | 14.17 | 3 | 3 | 7 | 8 | 607 | 65.3 | 5.38 |
| | OS = Citrus clementina | | | | | | | | |
| | GN = CICLE_v10027943mg | | | | | | | | |
| | PE = 3 SV = 1 - | | | | | | | | |
| | [V4RNH3_9ROSI] | | | | | | | | |
| V4SC53 | Uncharacterized protein | 15.42 | 5 | 2 | 8 | 12 | 655 | 72.8 | 5.27 |
| | OS = Citrus clementina | | | | | | | | |
| | GN = CICLE_v10027975mg | | | | | | | | |
| | PE = 3 SV = 1 - | | | | | | | | |
| | [V4SC53_9ROSI] | | | | | | | | |
| V4UFD6 | Uncharacterized protein | 30.05 | 12 | 3 | 14 | 36 | 649 | 71.0 | 5.21 |
| | OS = Citrus clementina | | | | | | | | |
| | GN = CICLE_v10027981mg | | | | | | | | |
| | PE = 3 SV = 1 - | | | | | | | | |
| | [V4UFD6_9ROSI] | | | | | | | | |
| V4SBI8 | Uncharacterized protein | 3.33 | 4 | 2 | 2 | 2 | 600 | 63.1 | 6.71 |
| | OS = Citrus clementina | | | | | | | | |
| | GN = CICLE_v10028055mg | | | | | | | | |
| | PE = 3 SV = 1 - | | | | | | | | |
| | [V4SBI8_9ROSI] | | | | | | | | |
| V4SB93 | Uncharacterized protein | 11.98 | 8 | 4 | 5 | 5 | 526 | 55.4 | 4.97 |
| | OS = Citrus clementina | | | | | | | | |
| | GN = CICLE_v10028085mg | | | | | | | | |
| | PE = 3 SV = 1 - | | | | | | | | |
| | [V4SB93_9ROSI] | | | | | | | | |
| V4SGB4 | Uncharacterized protein | 5.65 | 1 | 2 | 2 | 9 | 496 | 56.3 | 6.49 |
| | OS = Citrus clementina | | | | | | | | |
| | GN = CICLE_v10028266mg | | | | | | | | |
| | PE = 4 SV = 1 - | | | | | | | | |
| | [V4SGB4_9ROSI] | | | | | | | | |
| V4S6G6 | Uncharacterized protein | 16.22 | 16 | 6 | 6 | 7 | 450 | 49.6 | 5.06 |
| | OS = Citrus clementina | | | | | | | | |
| | GN = CICLE_v10028426mg | | | | | | | | |
| | PE = 3 SV = 1 - | | | | | | | | |
| | [V4S6G6_9ROSI] | | | | | | | | |
| V4SJ73 | Uncharacterized protein | 5.26 | 4 | 2 | 2 | 2 | 437 | 49.0 | 5.73 |
| | OS = Citrus clementina | | | | | | | | |
| | GN = CICLE_v10028480mg | | | | | | | | |
| | PE = 4 SV = 1 - | | | | | | | | |
| | [V4SJ73_9ROSI] | | | | | | | | |
| V4UEM7 | Uncharacterized protein | 5.96 | 6 | 1 | 1 | 1 | 235 | 26.4 | 8.78 |
| | OS = Citrus clementina | | | | | | | | |
| | GN = CICLE_v10028528mg | | | | | | | | |
| | PE = 4 SV = 1 - | | | | | | | | |
| | [V4UEM7_9ROSI] | | | | | | | | |
| V4UE78 | Uncharacterized protein | 13.85 | 3 | 4 | 4 | 4 | 390 | 43.6 | 6.15 |
| | OS = Citrus clementina | | | | | | | | |
| | GN = CICLE_v10028621mg | | | | | | | | |
| | PE = 4 SV = 1 - | | | | | | | | |
| | [V4UE78_9ROSI] | | | | | | | | |
| V4SJZ8 | Uncharacterized protein | 5.94 | 2 | 1 | 1 | 1 | 320 | 34.3 | 5.68 |
| | OS = Citrus clementina | | | | | | | | |
| | GN = CICLE_v10028755mg | | | | | | | | |
| | PE = 4 SV = 1 - | | | | | | | | |
| | [V4SJZ8_9ROSI] | | | | | | | | |
| V4S9D1 | Uncharacterized protein | 8.81 | 1 | 2 | 2 | 4 | 227 | 25.2 | 8.78 |
| | OS = Citrus clementina | | | | | | | | |
| | GN = CICLE_v10029222mg | | | | | | | | |
| | PE = 3 SV = 1 - | | | | | | | | |
| | [V4S9D1_9ROSI] | | | | | | | | |
| V4SLT9 | Uncharacterized protein | 20.25 | 4 | 2 | 3 | 9 | 158 | 17.9 | 7.42 |
| | OS = Citrus clementina | | | | | | | | |
| | GN = CICLE_v10029467mg | | | | | | | | |
| | PE = 3 SV = 1 - | | | | | | | | |
| | [V4SLT9_9ROSI] | | | | | | | | |
| V4UFW5 | Uncharacterized protein | 28.00 | 9 | 2 | 4 | 10 | 150 | 17.1 | 6.60 |
| | OS = Citrus clementina | | | | | | | | |
| | GN = CICLE_v10029495mg | | | | | | | | |
| | PE = 3 SV = 1 - | | | | | | | | |
| | [V4UFW5_9ROSI] | | | | | | | | |
| V4S723 | Uncharacterized protein | 21.62 | 2 | 3 | 3 | 4 | 148 | 16.8 | 10.23 |
| | OS = Citrus clementina | | | | | | | | |
| | GN = CICLE_v10029508mg | | | | | | | | |
| | PE = 3 SV = 1 - | | | | | | | | |
| | [V4S723_9ROSI] | | | | | | | | |
| V4SAR4 | Uncharacterized protein | 14.74 | 1 | 1 | 2 | 3 | 156 | 18.2 | 6.37 |
| | OS = Citrus clementina | | | | | | | | |
| | GN = CICLE_v10030303mg | | | | | | | | |
| | PE = 3 SV = 1 - | | | | | | | | |
| | [V4SAR4_9ROSI] | | | | | | | | |
| V4TQM7 | Uncharacterized protein | 0.64 | 1 | 1 | 1 | 1 | 1094 | 118.3 | 5.36 |
| | OS = Citrus clementina | | | | | | | | |
| | GN = CICLE_v10030570mg | | | | | | | | |
| | PE = 4 SV = 1 - | | | | | | | | |
| | [V4TQM7_9ROSI] | | | | | | | | |
| V4TKD6 | Uncharacterized protein | 3.29 | 11 | 3 | 3 | 3 | 881 | 95.9 | 6.11 |
| | OS = Citrus clementina | | | | | | | | |
| | GN = CICLE_v10030606mg | | | | | | | | |
| | PE = 4 SV = 1 - | | | | | | | | |
| | [V4TKD6_9ROSI] | | | | | | | | |
| V4ST94 | Uncharacterized protein | 7.94 | 5 | 2 | 4 | 4 | 793 | 90.1 | 5.10 |
| | OS = Citrus clementina | | | | | | | | |
| | GN = CICLE_v10030743mg | | | | | | | | |
| | PE = 3 SV = 1 - | | | | | | | | |
| | [V4ST94_9ROSI] | | | | | | | | |
| V4SSX5 | Uncharacterized protein | 3.75 | 8 | 1 | 2 | 2 | 587 | 65.7 | 5.10 |
| | OS = Citrus clementina | | | | | | | | |
| | GN = CICLE_v10031047mg | | | | | | | | |
| | PE = 4 SV = 1 - | | | | | | | | |
| | [V4SSX5_9ROSI] | | | | | | | | |
| V4TAG7 | Uncharacterized protein | 1.41 | 4 | 1 | 1 | 1 | 498 | 56.5 | 4.88 |
| | OS = Citrus clementina | | | | | | | | |
| | GN = CICLE_v10031185mg | | | | | | | | |
| | PE = 3 SV = 1 - | | | | | | | | |
| | [V4TAG7_9ROSI] | | | | | | | | |
| V4SU82 | Uncharacterized protein | 10.65 | 2 | 5 | 5 | 5 | 535 | 59.4 | 5.86 |
| | OS = Citrus clementina | | | | | | | | |
| | GN = CICLE_v10031191mg | | | | | | | | |
| | PE = 3 SV = 1 - | | | | | | | | |
| | [V4SU82_9ROSI] | | | | | | | | |
| V4SWD6 | Uncharacterized protein | 4.63 | 8 | 1 | 1 | 2 | 216 | 23.3 | 8.35 |
| | OS = Citrus clementina | | | | | | | | |
| | GN = CICLE_v10031842mg | | | | | | | | |
| | PE = 4 SV = 1 - | | | | | | | | |
| | [V4SWD6_9ROSI] | | | | | | | | |
| V4SNA8 | Uncharacterized protein | 9.47 | 17 | 2 | 2 | 2 | 264 | 29.9 | 7.12 |
| | OS = Citrus clementina | | | | | | | | |
| | GN = CICLE_v10031983mg | | | | | | | | |
| | PE = 4 SV = 1 - | | | | | | | | |
| | [V4SNA8_9ROSI] | | | | | | | | |
| V4SU16 | Uncharacterized protein | 6.50 | 2 | 2 | 2 | 2 | 323 | 34.1 | 9.48 |
| | OS = Citrus clementina | | | | | | | | |
| | GN = CICLE_v10032120mg | | | | | | | | |
| | PE = 3 SV = 1 - | | | | | | | | |
| | [V4SU16_9ROSI] | | | | | | | | |
| V4T785 | Uncharacterized protein | 8.30 | 2 | 2 | 2 | 2 | 289 | 32.8 | 5.17 |
| | OS = Citrus clementina | | | | | | | | |
| | GN = CICLE_v10032291mg | | | | | | | | |
| | PE = 4 SV = 1 - | | | | | | | | |
| | [V4T785_9ROSI] | | | | | | | | |
| V4SMQ3 | Uncharacterized protein | 6.27 | 1 | 1 | 1 | 1 | 287 | 30.4 | 6.57 |
| | OS = Citrus clementina | | | | | | | | |
| | GN = CICLE_v10032300mg | | | | | | | | |
| | PE = 4 SV = 1 - | | | | | | | | |
| | [V4SMQ3_9ROSI] | | | | | | | | |
| V4SVZ8 | Uncharacterized protein | 11.11 | 2 | 3 | 3 | 3 | 261 | 28.9 | 7.17 |
| | OS = Citrus clementina | | | | | | | | |
| | GN = CICLE_v10032479mg | | | | | | | | |
| | PE = 4 SV = 1 - | | | | | | | | |
| | [V4SVZ8_9ROSI] | | | | | | | | |
| V4TEY9 | Uncharacterized protein | 12.74 | 4 | 3 | 3 | 3 | 259 | 29.4 | 10.27 |
| | OS = Citrus clementina | | | | | | | | |
| | GN = CICLE_v10032494mg | | | | | | | | |
| | PE = 4 SV = 1 - | | | | | | | | |
| | [V4TEY9_9ROSI] | | | | | | | | |
| V4TIX1 | Uncharacterized protein | 3.38 | 1 | 1 | 1 | 1 | 237 | 26.3 | 6.20 |
| | OS = Citrus clementina | | | | | | | | |
| | GN = CICLE_v10032618mg | | | | | | | | |
| | PE = 4 SV = 1 - | | | | | | | | |
| | [V4TIX1_9ROSI] | | | | | | | | |
| V4SVI6 | Uncharacterized protein | 4.57 | 1 | 1 | 1 | 1 | 197 | 23.1 | 10.35 |
| | OS = Citrus clementina | | | | | | | | |
| | GN = CICLE_v10032800mg | | | | | | | | |
| | PE = 3 SV = 1 - | | | | | | | | |
| | [V4SVI6_9ROSI] | | | | | | | | |
| V4T891 | Uncharacterized protein | 11.56 | 4 | 2 | 2 | 2 | 147 | 17.1 | 5.02 |
| | OS = Citrus clementina | | | | | | | | |
| | GN = CICLE_v10032923mg | | | | | | | | |
| | PE = 4 SV = 1 - | | | | | | | | |
| | [V4T891_9ROSI] | | | | | | | | |
| V4TCL3 | Uncharacterized protein | 20.92 | 5 | 3 | 3 | 4 | 153 | 17.4 | 10.24 |
| | OS = Citrus clementina | | | | | | | | |
| | GN = CICLE_v10032980mg | | | | | | | | |
| | PE = 3 SV = 1 - | | | | | | | | |
| | [V4TCL3_9ROSI] | | | | | | | | |
| V4SNC1 | Uncharacterized protein | 23.30 | 2 | 3 | 3 | 3 | 103 | 11.7 | 10.13 |
| | OS = Citrus clementina | | | | | | | | |
| | GN = CICLE_v10033065mg | | | | | | | | |
| | PE = 4 SV = 1 - | | | | | | | | |
| | [V4SNC1_9ROSI] | | | | | | | | |
| V4TJF6 | Uncharacterized protein | 11.21 | 2 | 1 | 1 | 1 | 107 | 11.8 | 8.57 |
| | OS = Citrus clementina | | | | | | | | |
| | GN = CICLE_v10033115mg | | | | | | | | |
| | PE = 4 SV = 1 - | | | | | | | | |
| | [V4TJF6_9ROSI] | | | | | | | | |
| V4SSU2 | Uncharacterized protein | 3.39 | 3 | 3 | 3 | 3 | 915 | 102.1 | 6.98 |
| | OS = Citrus clementina | | | | | | | | |
| | GN = CICLE_v10033422mg | | | | | | | | |
| | PE = 3 SV = 1 - | | | | | | | | |
| | [V4SSU2_9ROSI] | | | | | | | | |
| V4TKX4 | Uncharacterized protein | 7.05 | 4 | 1 | 1 | 1 | 156 | 17.2 | 9.73 |
| | OS = Citrus clementina | | | | | | | | |
| | GN = CICLE_v10033520mg | | | | | | | | |
| | PE = 4 SV = 1 - | | | | | | | | |
| | [V4TKX4_9ROSI] | | | | | | | | |
| V4VHG6 | Uncharacterized protein | 16.60 | 2 | 2 | 4 | 4 | 235 | 27.0 | 8.97 |
| | OS = Citrus clementina | | | | | | | | |
| | GN = CICLE_v10033567mg | | | | | | | | |
| | PE = 3 SV = 1 - | | | | | | | | |
| | [V4VHG6_9ROSI] | | | | | | | | |
| V4T7S4 | Uncharacterized protein | 24.50 | 8 | 1 | 12 | 32 | 653 | 71.4 | 5.29 |
| | OS = Citrus clementina | | | | | | | | |
| | GN = CICLE_v10033622mg | | | | | | | | |
| | PE = 3 SV = 1 - | | | | | | | | |
| | [V4T7S4_9ROSI] | | | | | | | | |
| A0A067ENH2 | Uncharacterized protein | 0.64 | 5 | 1 | 1 | 1 | 1866 | 201.6 | 6.86 |
| | OS = Citrus sinensis | | | | | | | | |
| | GN = CISIN_1g000049mg | | | | | | | | |
| | PE = 4 SV = 1 - | | | | | | | | |
| | [A0A067ENH2_CITSI] | | | | | | | | |
| A0A067FP30 | Uncharacterized protein | 1.61 | 3 | 1 | 1 | 1 | 1057 | 115.9 | 5.62 |
| | OS = Citrus sinensis | | | | | | | | |
| | GN = CISIN_1g000572mg | | | | | | | | |
| | PE = 4 SV = 1 - | | | | | | | | |
| | [A0A067FP30_CITSI] | | | | | | | | |
| A0A067DGR7 | Uncharacterized protein | 1.54 | 4 | 2 | 2 | 2 | 1167 | 128.7 | 5.74 |
| | OS = Citrus sinensis | | | | | | | | |
| | GN = CISIN_1g000934mg | | | | | | | | |
| | PE = 4 SV = 1 - | | | | | | | | |
| | [A0A067DGR7_CITSI] | | | | | | | | |
| A0A067FKC9 | Uncharacterized protein | 2.23 | 2 | 2 | 2 | 2 | 1213 | 134.4 | 5.48 |
| | OS = Citrus sinensis | | | | | | | | |
| | GN = CISIN_1g000944mg | | | | | | | | |
| | PE = 4 SV = 1 - | | | | | | | | |
| | [A0A067FKC9_CITSI] | | | | | | | | |
| A0A067FU63 | Uncharacterized protein | 4.22 | 11 | 4 | 4 | 4 | 972 | 107.6 | 5.33 |
| | OS = Citrus sinensis | | | | | | | | |
| | GN = CISIN_1g001074mg | | | | | | | | |
| | PE = 3 SV = 1 - | | | | | | | | |
| | [A0A067FU63_CITSI] | | | | | | | | |
| A0A067EDX8 | Uncharacterized protein | 1.02 | 3 | 1 | 1 | 1 | 1075 | 115.4 | 7.30 |
| | OS = Citrus sinensis | | | | | | | | |
| | GN = CISIN_1g001219mg | | | | | | | | |
| | PE = 4 SV = 1 - | | | | | | | | |
| | [A0A067EDX8_CITSI] | | | | | | | | |
| A0A067GJ31 | Uncharacterized protein | 1.42 | 4 | 1 | 1 | 1 | 774 | 86.6 | 5.27 |
| | OS = Citrus sinensis | | | | | | | | |
| | GN = CISIN_1g001387mg | | | | | | | | |
| | PE = 4 SV = 1 - | | | | | | | | |
| | [A0A067GJ31_CITSI] | | | | | | | | |
| A0A067E232 | Uncharacterized protein | 1.58 | 4 | 1 | 1 | 1 | 821 | 91.6 | 5.43 |
| | OS = Citrus sinensis | | | | | | | | |
| | GN = CISIN_1g001822mg | | | | | | | | |
| | PE = 4 SV = 1 - | | | | | | | | |
| | [A0A067E232_CITSI] | | | | | | | | |
| A0A067FD92 | Uncharacterized protein | 4.00 | 3 | 3 | 3 | 3 | 1001 | 109.1 | 5.36 |
| | OS = Citrus sinensis | | | | | | | | |
| | GN = CISIN_1g001859mg | | | | | | | | |
| | PE = 4 SV = 1 - | | | | | | | | |
| | [A0A067FD92_CITSI] | | | | | | | | |
| A0A067FW96 | Uncharacterized protein | 5.91 | 3 | 3 | 5 | 5 | 982 | 110.0 | 7.23 |
| | OS = Citrus sinensis | | | | | | | | |
| | GN = CISIN_1g002012mg | | | | | | | | |
| | PE = 3 SV = 1 - | | | | | | | | |
| | [A0A067FW96_CITSI] | | | | | | | | |
| A0A067DSV7 | Uncharacterized protein | 16.41 | 10 | 5 | 14 | 19 | 969 | 111.0 | 6.55 |
| | OS = Citrus sinensis | | | | | | | | |
| | GN = CISIN_1g002089mg | | | | | | | | |
| | PE = 3 SV = 1 - | | | | | | | | |
| | [A0A067DSV7_CITSI] | | | | | | | | |
| A0A067F7J8 | Uncharacterized protein | 16.99 | 7 | 6 | 15 | 18 | 965 | 110.1 | 6.60 |
| | OS = Citrus sinensis | | | | | | | | |
| | GN = CISIN_1g002112mg | | | | | | | | |
| | PE = 3 SV = 1 - | | | | | | | | |
| | [A0A067F7J8_CITSI] | | | | | | | | |
| A0A067E4P9 | Uncharacterized protein | 0.92 | 4 | 1 | 1 | 1 | 871 | 98.7 | 5.49 |
| | OS = Citrus sinensis | | | | | | | | |
| | GN = CISIN_1g002496mg | | | | | | | | |
| | PE = 4 SV = 1 - | | | | | | | | |
| | [A0A067E4P9_CITSI] | | | | | | | | |
| A0A067G4Z4 | Uncharacterized protein | 4.00 | 19 | 3 | 3 | 3 | 775 | 87.1 | 5.59 |
| | OS = Citrus sinensis | | | | | | | | |
| | GN = CISIN_1g002775mg | | | | | | | | |
| | PE = 4 SV = 1 - | | | | | | | | |
| | [A0A067G4Z4_CITSI] | | | | | | | | |
| A0A067GAC9 | Uncharacterized protein | 3.68 | 5 | 2 | 2 | 2 | 653 | 72.5 | 4.84 |
| | OS = Citrus sinensis | | | | | | | | |
| | GN = CISIN_1g002876mg | | | | | | | | |
| | PE = 4 SV = 1 - | | | | | | | | |
| | [A0A067GAC9_CITSI] | | | | | | | | |
| A0A067EBF9 | Uncharacterized protein | 3.03 | 4 | 3 | 3 | 4 | 726 | 80.4 | 5.26 |
| | OS = Citrus sinensis | | | | | | | | |
| | GN = CISIN_1g003056mg | | | | | | | | |
| | PE = 3 SV = 1 - | | | | | | | | |
| | [A0A067EBF9_CITSI] | | | | | | | | |
| A0A067EK53 | Uncharacterized protein | 8.08 | 3 | 3 | 4 | 4 | 693 | 77.2 | 5.38 |
| | OS = Citrus sinensis | | | | | | | | |
| | GN = CISIN_1g003094mg | | | | | | | | |
| | PE = 3 SV = 1 - | | | | | | | | |
| | [A0A067EK53_CITSI] | | | | | | | | |
| A0A067E683 | Uncharacterized protein | 12.26 | 5 | 2 | 8 | 9 | 832 | 92.5 | 6.57 |
| | OS = Citrus sinensis | | | | | | | | |
| | GN = CISIN_1g003165mg | | | | | | | | |
| | PE = 4 SV = 1 - | | | | | | | | |
| | [A0A067E683_CITSI] | | | | | | | | |
| A0A067DWB5 | Uncharacterized protein | 10.95 | 6 | 1 | 7 | 8 | 831 | 92.8 | 6.11 |
| | OS = Citrus sinensis | | | | | | | | |
| | GN = CISIN_1g003169mg | | | | | | | | |
| | PE = 4 SV = 1 - | | | | | | | | |
| | [A0A067DWB5_CITSI] | | | | | | | | |
| A0A067FHG1 | Uncharacterized protein | 5.18 | 11 | 1 | 2 | 2 | 463 | 52.4 | 6.61 |
| | OS = Citrus sinensis | | | | | | | | |
| | GN = CISIN_1g003196mg | | | | | | | | |
| | PE = 3 SV = 1 - | | | | | | | | |
| | [A0A067FHG1_CITSI] | | | | | | | | |
| A0A067HB36 | Uncharacterized protein | 8.14 | 6 | 5 | 6 | 7 | 762 | 87.9 | 5.27 |
| | OS = Citrus sinensis | | | | | | | | |
| | GN = CISIN_1g003458mg | | | | | | | | |
| | PE = 3 SV = 1 - | | | | | | | | |
| | [A0A067HB36_CITSI] | | | | | | | | |
| A0A067F4C9 | Uncharacterized protein | 1.98 | 5 | 1 | 1 | 1 | 504 | 55.3 | 6.25 |
| | OS = Citrus sinensis | | | | | | | | |
| | GN = CISIN_1g003550mg | | | | | | | | |
| | PE = 4 SV = 1 - | | | | | | | | |
| | [A0A067F4C9_CITSI] | | | | | | | | |
| A0A067EYR0 | Uncharacterized protein | 22.19 | 7 | 3 | 14 | 15 | 757 | 84.1 | 5.29 |
| | OS = Citrus sinensis | | | | | | | | |
| | GN = CISIN_1g003620mg | | | | | | | | |
| | PE = 3 SV = 1 - | | | | | | | | |
| | [A0A067EYR0_CITSI] | | | | | | | | |
| A0A067ETJ5 | Uncharacterized protein | 7.70 | 3 | 1 | 4 | 4 | 753 | 85.8 | 5.38 |
| | OS = Citrus sinensis | | | | | | | | |
| | GN = CISIN_1g003713mg | | | | | | | | |
| | PE = 3 SV = 1 - | | | | | | | | |
| | [A0A067ETJ5_CITSI] | | | | | | | | |
| A0A067F2P8 | Uncharacterized protein | 1.70 | 9 | 1 | 1 | 1 | 763 | 87.9 | 8.98 |
| | OS = Citrus sinensis | | | | | | | | |
| | GN = CISIN_1g004109mg | | | | | | | | |
| | PE = 4 SV = 1 - | | | | | | | | |
| | [A0A067F2P8_CITSI] | | | | | | | | |
| A0A067FP05 | Uncharacterized protein | 1.42 | 2 | 1 | 1 | 1 | 772 | 88.1 | 9.01 |
| | OS = Citrus sinensis | | | | | | | | |
| | GN = CISIN_1g004127mg | | | | | | | | |
| | PE = 4 SV = 1 - | | | | | | | | |
| | [A0A067FP05_CITSI] | | | | | | | | |
| A0A067DL06 | Uncharacterized protein | 3.20 | 9 | 2 | 2 | 2 | 563 | 61.6 | 9.38 |
| | OS = Citrus sinensis | | | | | | | | |
| | GN = CISIN_1g004726mg | | | | | | | | |
| | PE = 4 SV = 1 - | | | | | | | | |
| | [A0A067DL06_CITSI] | | | | | | | | |
| A0A067GIW8 | Uncharacterized protein | 2.37 | 10 | 1 | 1 | 1 | 379 | 42.7 | 8.82 |
| | OS = Citrus sinensis | | | | | | | | |
| | GN = CISIN_1g004795mg | | | | | | | | |
| | PE = 4 SV = 1 - | | | | | | | | |
| | [A0A067GIW8_CITSI] | | | | | | | | |
| A0A067DN94 | Uncharacterized protein | 6.16 | 2 | 1 | 4 | 4 | 714 | 76.6 | 5.45 |
| | OS = Citrus sinensis | | | | | | | | |
| | GN = CISIN_1g005099mg | | | | | | | | |
| | PE = 3 SV = 1 - | | | | | | | | |
| | [A0A067DN94_CITSI] | | | | | | | | |
| A0A067G9H4 | Uncharacterized protein | 20.94 | 2 | 4 | 14 | 25 | 702 | 80.6 | 5.07 |
| | OS = Citrus sinensis | | | | | | | | |
| | GN = CISIN_1g005321mg | | | | | | | | |
| | PE = 3 SV = 1 - | | | | | | | | |
| | [A0A067G9H4_CITSI] | | | | | | | | |
| A0A067F0T0 | Uncharacterized protein | 20.46 | 3 | 4 | 14 | 23 | 699 | 80.6 | 5.10 |
| | OS = Citrus sinensis | | | | | | | | |
| | GN = CISIN_1g005370mg | | | | | | | | |
| | PE = 3 SV = 1 - | | | | | | | | |
| | [A0A067F0T0_CITSI] | | | | | | | | |
| A0A067F1P0 | Uncharacterized protein | 1.92 | 5 | 1 | 1 | 1 | 574 | 64.4 | 9.28 |
| | OS = Citrus sinensis | | | | | | | | |
| | GN = CISIN_1g005564mg | | | | | | | | |
| | PE = 4 SV = 1 - | | | | | | | | |
| | [A0A067F1P0_CITSI] | | | | | | | | |
| A0A067EQV8 | Uncharacterized protein | 13.85 | 4 | 1 | 7 | 11 | 657 | 72.7 | 5.31 |
| | OS = Citrus sinensis | | | | | | | | |
| | GN = CISIN_1g006194mg | | | | | | | | |
| | PE = 3 SV = 1 - | | | | | | | | |
| | [A0A067EQV8_CITSI] | | | | | | | | |
| A0A067GP41 | Uncharacterized protein | 3.42 | 7 | 1 | 1 | 1 | 468 | 51.3 | 6.80 |
| | OS = Citrus sinensis | | | | | | | | |
| | GN = CISIN_1g006297mg | | | | | | | | |
| | PE = 4 SV = 1 - | | | | | | | | |
| | [A0A067GP41_CITSI] | | | | | | | | |
| A0A067HA13 | Uncharacterized protein | 1.52 | 5 | 1 | 1 | 1 | 592 | 66.9 | 5.67 |
| | OS = Citrus sinensis | | | | | | | | |
| | GN = CISIN_1g006318mg | | | | | | | | |
| | PE = 4 SV = 1 - | | | | | | | | |
| | [A0A067HA13_CITSI] | | | | | | | | |
| A0A067EYI3 | Uncharacterized protein | 2.20 | 2 | 1 | 1 | 1 | 635 | 68.9 | 7.53 |
| | OS = Citrus sinensis | | | | | | | | |
| | GN = CISIN_1g006678mg | | | | | | | | |
| | PE = 4 SV = 1 - | | | | | | | | |
| | [A0A067EYI3_CITSI] | | | | | | | | |
| A0A067H2Y4 | Uncharacterized protein | 12.59 | 6 | 7 | 7 | 8 | 588 | 64.0 | 6.79 |
| | OS = Citrus sinensis | | | | | | | | |
| | GN = CISIN_1g006964mg | | | | | | | | |
| | PE = 3 SV = 1 - | | | | | | | | |
| | [A0A067H2Y4_CITSI] | | | | | | | | |
| A0A067FGU3 | Uncharacterized protein | 2.11 | 11 | 1 | 1 | 1 | 426 | 48.1 | 6.38 |
| | OS = Citrus sinensis | | | | | | | | |
| | GN = CISIN_1g007251mg | | | | | | | | |
| | PE = 3 SV = 1 - | | | | | | | | |
| | [A0A067FGU3_CITSI] | | | | | | | | |
| A0A067F6B2 | Uncharacterized protein | 1.97 | 4 | 1 | 1 | 1 | 609 | 66.2 | 7.18 |
| | OS = Citrus sinensis | | | | | | | | |
| | GN = CISIN_1g007283mg | | | | | | | | |
| | PE = 4 SV = 1 - | | | | | | | | |
| | [A0A067F6B2_CITSI] | | | | | | | | |
| A0A067EU67 | Uncharacterized protein | 19.77 | 7 | 9 | 9 | 10 | 531 | 56.5 | 7.72 |
| | OS = Citrus sinensis | | | | | | | | |
| | GN = CISIN_1g007313mg | | | | | | | | |
| | PE = 3 SV = 1 - | | | | | | | | |
| | [A0A067EU67_CITSI] | | | | | | | | |
| A0A067G0L3 | Uncharacterized protein | 5.81 | 4 | 3 | 3 | 4 | 602 | 65.3 | 7.68 |
| | OS = Citrus sinensis | | | | | | | | |
| | GN = CISIN_1g007327mg | | | | | | | | |
| | PE = 4 SV = 1 - | | | | | | | | |
| | [A0A067G0L3_CITSI] | | | | | | | | |
| A0A067FU03 | Uncharacterized protein | 4.22 | 7 | 2 | 2 | 2 | 474 | 53.2 | 6.49 |
| | OS = Citrus sinensis | | | | | | | | |
| | GN = CISIN_1g007799mg | | | | | | | | |
| | PE = 4 SV = 1 - | | | | | | | | |
| | [A0A067FU03_CITSI] | | | | | | | | |
| A0A067G108 | Uncharacterized protein | 4.17 | 7 | 1 | 1 | 1 | 312 | 34.9 | 5.43 |
| | OS = Citrus sinensis | | | | | | | | |
| | GN = CISIN_1g007850mg | | | | | | | | |
| | PE = 4 SV = 1 - | | | | | | | | |
| | [A0A067G108_CITSI] | | | | | | | | |
| A0A067DTP5 | Uncharacterized protein | 8.01 | 8 | 1 | 2 | 2 | 337 | 37.7 | 5.74 |
| | OS = Citrus sinensis | | | | | | | | |
| | GN = CISIN_1g007863mg | | | | | | | | |
| | PE = 4 SV = 1 - | | | | | | | | |
| | [A0A067DTP5_CITSI] | | | | | | | | |
| A0A067EUD8 | Uncharacterized protein | 5.07 | 7 | 2 | 2 | 2 | 434 | 47.1 | 6.44 |
| | OS = Citrus sinensis | | | | | | | | |
| | GN = CISIN_1g007951mg | | | | | | | | |
| | PE = 4 SV = 1 - | | | | | | | | |
| | [A0A067EUD8_CITSI] | | | | | | | | |
| A0A067EMC7 | Uncharacterized protein | 10.66 | 5 | 4 | 4 | 4 | 544 | 58.5 | 5.20 |
| | OS = Citrus sinensis | | | | | | | | |
| | GN = CISIN_1g008377mg | | | | | | | | |
| | PE = 3 SV = 1 - | | | | | | | | |
| | [A0A067EMC7_CITSI] | | | | | | | | |
| A0A067EL65 | Uncharacterized protein | 3.00 | 9 | 2 | 2 | 2 | 567 | 63.8 | 6.07 |
| | OS = Citrus sinensis | | | | | | | | |
| | GN = CISIN_1g008405mg | | | | | | | | |
| | PE = 3 SV = 1 - | | | | | | | | |
| | [A0A067EL65_CITSI] | | | | | | | | |
| A0A067E1D3 | Uncharacterized protein | 10.90 | 9 | 4 | 4 | 5 | 422 | 45.8 | 6.37 |
| | OS = Citrus sinensis | | | | | | | | |
| | GN = CISIN_1g008527mg | | | | | | | | |
| | PE = 3 SV = 1 - | | | | | | | | |
| | [A0A067E1D3_CITSI] | | | | | | | | |
| A0A067G043 | Uncharacterized protein | 3.63 | 8 | 2 | 2 | 2 | 496 | 55.0 | 9.39 |
| | OS = Citrus sinensis | | | | | | | | |
| | GN = CISIN_1g008576mg | | | | | | | | |
| | PE = 4 SV = 1 - | | | | | | | | |
| | [A0A067G043_CITSI] | | | | | | | | |
| A0A067FA30 | Uncharacterized protein | 9.74 | 5 | 2 | 2 | 2 | 421 | 47.0 | 6.68 |
| | OS = Citrus sinensis | | | | | | | | |
| | GN = CISIN_1g008774mg | | | | | | | | |
| | PE = 4 SV = 1 - | | | | | | | | |
| | [A0A067FA30_CITSI] | | | | | | | | |
| A0A067E9H4 | Uncharacterized protein | 2.91 | 4 | 1 | 1 | 1 | 549 | 60.1 | 6.61 |
| | OS = Citrus sinensis | | | | | | | | |
| | GN = CISIN_1g008878mg | | | | | | | | |
| | PE = 4 SV = 1 - | | | | | | | | |
| | [A0A067E9H4_CITSI] | | | | | | | | |
| A0A067GNU3 | Uncharacterized protein | 2.47 | 3 | 1 | 1 | 1 | 445 | 50.0 | 7.81 |
| | OS = Citrus sinensis | | | | | | | | |
| | GN = CISIN_1g008945mg | | | | | | | | |
| | PE = 4 SV = 1 - | | | | | | | | |
| | [A0A067GNU3_CITSI] | | | | | | | | |
| A0A067GWK0 | Uncharacterized protein | 1.85 | 5 | 1 | 1 | 1 | 433 | 49.1 | 5.38 |
| | OS = Citrus sinensis | | | | | | | | |
| | GN = CISIN_1g009270mg | | | | | | | | |
| | PE = 4 SV = 1 - | | | | | | | | |
| | [A0A067GWK0_CITSI] | | | | | | | | |
| A0A067EBU4 | Uncharacterized protein | 8.76 | 3 | 4 | 4 | 4 | 525 | 59.3 | 5.73 |
| | OS = Citrus sinensis | | | | | | | | |
| | GN = CISIN_1g009278mg | | | | | | | | |
| | PE = 4 SV = 1 - | | | | | | | | |
| | [A0A067EBU4_CITSI] | | | | | | | | |
| A0A067G8V5 | Uncharacterized protein | 6.79 | 8 | 2 | 2 | 2 | 368 | 40.8 | 7.72 |
| | OS = Citrus sinensis | | | | | | | | |
| | GN = CISIN_1g009544mg | | | | | | | | |
| | PE = 4 SV = 1 - | | | | | | | | |
| | [A0A067G8V5_CITSI] | | | | | | | | |
| A0A067G2S7 | Uncharacterized protein | 3.50 | 11 | 1 | 1 | 1 | 314 | 34.1 | 8.43 |
| | OS = Citrus sinensis | | | | | | | | |
| | GN = CISIN_1g009582mg | | | | | | | | |
| | PE = 4 SV = 1 - | | | | | | | | |
| | [A0A067G2S7_CITSI] | | | | | | | | |
| A0A067H268 | Uncharacterized protein | 7.50 | 3 | 3 | 3 | 3 | 507 | 54.8 | 5.78 |
| | OS = Citrus sinensis | | | | | | | | |
| | GN = CISIN_1g009718mg | | | | | | | | |
| | PE = 3 SV = 1 - | | | | | | | | |
| | [A0A067H268_CITSI] | | | | | | | | |
| A0A067FCR2 | Uncharacterized protein | 5.32 | 4 | 2 | 2 | 2 | 526 | 57.7 | 8.15 |
| | OS = Citrus sinensis | | | | | | | | |
| | GN = CISIN_1g009781mg | | | | | | | | |
| | PE = 4 SV = 1 - | | | | | | | | |
| | [A0A067FCR2_CITSI] | | | | | | | | |
| A0A067E4I5 | Uncharacterized protein | 5.76 | 2 | 2 | 2 | 2 | 521 | 56.1 | 4.74 |
| | OS = Citrus sinensis | | | | | | | | |
| | GN = CISIN_1g009973mg | | | | | | | | |
| | PE = 4 SV = 1 - | | | | | | | | |
| | [A0A067E4I5_CITSI] | | | | | | | | |
| A0A067GS99 | Uncharacterized protein | 1.91 | 4 | 1 | 1 | 1 | 471 | 51.2 | 5.74 |
| | OS = Citrus sinensis | | | | | | | | |
| | GN = CISIN_1g010233mg | | | | | | | | |
| | PE = 3 SV = 1 - | | | | | | | | |
| | [A0A067GS99_CITSI] | | | | | | | | |
| A0A067DPR9 | Uncharacterized protein | 2.05 | 5 | 1 | 1 | 1 | 440 | 48.7 | 6.79 |
| | OS = Citrus sinensis | | | | | | | | |
| | GN = CISIN_1g010403mg | | | | | | | | |
| | PE = 4 SV = 1 - | | | | | | | | |
| | [A0A067DPR9_CITSI] | | | | | | | | |
| A0A067F2V2 | Uncharacterized protein | 1.77 | 4 | 1 | 1 | 1 | 453 | 49.8 | 7.28 |
| | OS = Citrus sinensis | | | | | | | | |
| | GN = CISIN_1g010453mg | | | | | | | | |
| | PE = 4 SV = 1 - | | | | | | | | |
| | [A0A067F2V2_CITSI] | | | | | | | | |
| A0A067FKC2 | Uncharacterized protein | 9.51 | 10 | 2 | 2 | 2 | 284 | 31.1 | 6.38 |
| | OS = Citrus sinensis | | | | | | | | |
| | GN = CISIN_1g010895mg | | | | | | | | |
| | PE = 3 SV = 1 - | | | | | | | | |
| | [A0A067FKC2_CITSI] | | | | | | | | |
| A0A067FN97 | Uncharacterized protein | 2.54 | 5 | 1 | 1 | 1 | 354 | 40.3 | 7.15 |
| | OS = Citrus sinensis | | | | | | | | |
| | GN = CISIN_1g010941mg | | | | | | | | |
| | PE = 4 SV = 1 - | | | | | | | | |
| | [A0A067FN97_CITSI] | | | | | | | | |
| A0A067EBQ2 | Uncharacterized protein | 6.09 | 2 | 3 | 3 | 3 | 493 | 54.3 | 6.93 |
| | OS = Citrus sinensis | | | | | | | | |
| | GN = CISIN_1g011097mg | | | | | | | | |
| | PE = 3 SV = 1 - | | | | | | | | |
| | [A0A067EBQ2_CITSI] | | | | | | | | |
| A0A067GPH0 | Uncharacterized protein | 3.90 | 10 | 1 | 1 | 1 | 282 | 30.8 | 9.39 |
| | OS = Citrus sinensis | | | | | | | | |
| | GN = CISIN_1g011107mg | | | | | | | | |
| | PE = 3 SV = 1 - | | | | | | | | |
| | [A0A067GPH0_CITSI] | | | | | | | | |
| A0A067FDB9 | Uncharacterized protein | 2.03 | 2 | 1 | 1 | 1 | 492 | 55.3 | 6.67 |
| | OS = Citrus sinensis | | | | | | | | |
| | GN = CISIN_1g011170mg | | | | | | | | |
| | PE = 4 SV = 1 - | | | | | | | | |
| | [A0A067FDB9_CITSI] | | | | | | | | |
| A0A067GT82 | Uncharacterized protein | 5.39 | 5 | 1 | 1 | 1 | 297 | 31.2 | 5.15 |
| | OS = Citrus sinensis | | | | | | | | |
| | GN = CISIN_1g011306mg | | | | | | | | |
| | PE = 4 SV = 1 - | | | | | | | | |
| | [A0A067GT82_CITSI] | | | | | | | | |
| A0A067FY35 | Uncharacterized protein | 1.77 | 2 | 1 | 1 | 1 | 453 | 51.7 | 9.06 |
| | OS = Citrus sinensis | | | | | | | | |
| | GN = CISIN_1g011352mg | | | | | | | | |
| | PE = 4 SV = 1 - | | | | | | | | |
| | [A0A067FY35_CITSI] | | | | | | | | |
| A0A067FRJ0 | Uncharacterized protein | 5.75 | 3 | 2 | 2 | 2 | 452 | 46.9 | 9.23 |
| | OS = Citrus sinensis | | | | | | | | |
| | GN = CISIN_1g011684mg | | | | | | | | |
| | PE = 3 SV = 1 - | | | | | | | | |
| | [A0A067FRJ0_CITSI] | | | | | | | | |
| A0A067ETI5 | Uncharacterized protein | 4.80 | 4 | 1 | 1 | 1 | 333 | 37.3 | 5.21 |
| | OS = Citrus sinensis | | | | | | | | |
| | GN = CISIN_1g011768mg | | | | | | | | |
| | PE = 4 SV = 1 - | | | | | | | | |
| | [A0A067ETI5_CITSI] | | | | | | | | |
| A0A067DM36 | Uncharacterized protein | 6.08 | 4 | 2 | 2 | 2 | 477 | 51.7 | 8.16 |
| | OS = Citrus sinensis | | | | | | | | |
| | GN = CISIN_1g011777mg | | | | | | | | |
| | PE = 3 SV = 1 - | | | | | | | | |
| | [A0A067DM36_CITSI] | | | | | | | | |
| A0A067GRF9 | Uncharacterized protein | 3.50 | 9 | 1 | 1 | 1 | 314 | 34.4 | 9.16 |
| | OS = Citrus sinensis | | | | | | | | |
| | GN = CISIN_1g011896mg | | | | | | | | |
| | PE = 3 SV = 1 - | | | | | | | | |
| | [A0A067GRF9_CITSI] | | | | | | | | |
| A0A067FHG9 | Uncharacterized protein | 13.77 | 3 | 4 | 4 | 6 | 472 | 50.5 | 5.71 |
| | OS = Citrus sinensis | | | | | | | | |
| | GN = CISIN_1g012041mg | | | | | | | | |
| | PE = 3 SV = 1 - | | | | | | | | |
| | [A0A067FHG9_CITSI] | | | | | | | | |
| A0A067EU33 | Uncharacterized protein | 4.56 | 8 | 2 | 2 | 2 | 373 | 41.1 | 9.04 |
| | OS = Citrus sinensis | | | | | | | | |
| | GN = CISIN_1g012142mg | | | | | | | | |
| | PE = 3 SV = 1 - | | | | | | | | |
| | [A0A067EU33_CITSI] | | | | | | | | |
| A0A067GEK0 | Uncharacterized protein | 7.91 | 2 | 2 | 2 | 2 | 468 | 49.8 | 7.46 |
| | OS = Citrus sinensis | | | | | | | | |
| | GN = CISIN_1g012221mg | | | | | | | | |
| | PE = 3 SV = 1 - | | | | | | | | |
| | [A0A067GEK0_CITSI] | | | | | | | | |
| A0A067ESV8 | Uncharacterized protein | 1.73 | 3 | 1 | 1 | 1 | 462 | 51.0 | 9.45 |
| | OS = Citrus sinensis | | | | | | | | |
| | GN = CISIN_1g012495mg | | | | | | | | |
| | PE = 4 SV = 1 - | | | | | | | | |
| | [A0A067ESV8_CITSI] | | | | | | | | |
| A0A067DT17 | Uncharacterized protein | 2.17 | 2 | 1 | 1 | 1 | 461 | 49.6 | 5.94 |
| | OS = Citrus sinensis | | | | | | | | |
| | GN = CISIN_1g012505mg | | | | | | | | |
| | PE = 4 SV = 1 - | | | | | | | | |
| | [A0A067DT17_CITSI] | | | | | | | | |
| A0A067DX62 | Uncharacterized protein | 14.78 | 14 | 4 | 4 | 5 | 345 | 36.2 | 8.09 |
| | OS = Citrus sinensis | | | | | | | | |
| | GN = CISIN_1g012506mg | | | | | | | | |
| | PE = 3 SV = 1 - | | | | | | | | |
| | [A0A067DX62_CITSI] | | | | | | | | |
| A0A067GSL3 | Uncharacterized protein | 3.07 | 7 | 1 | 1 | 1 | 326 | 35.8 | 5.53 |
| | OS = Citrus sinensis | | | | | | | | |
| | GN = CISIN_1g012786mg | | | | | | | | |
| | PE = 4 SV = 1 - | | | | | | | | |
| | [A0A067GSL3_CITSI] | | | | | | | | |
| A0A067FQS6 | Uncharacterized protein | 7.63 | 14 | 3 | 3 | 4 | 393 | 42.7 | 6.62 |
| | OS = Citrus sinensis | | | | | | | | |
| | GN = CISIN_1g012806mg | | | | | | | | |
| | PE = 3 SV = 1 - | | | | | | | | |
| | [A0A067FQS6_CITSI] | | | | | | | | |
| A0A067FZE1 | Uncharacterized protein | 11.52 | 3 | 3 | 3 | 3 | 330 | 36.1 | 6.02 |
| | OS = Citrus sinensis | | | | | | | | |
| | GN = CISIN_1g013007mg | | | | | | | | |
| | PE = 4 SV = 1 - | | | | | | | | |
| | [A0A067FZE1_CITSI] | | | | | | | | |
| A0A067EUE0 | Uncharacterized protein | 4.43 | 3 | 1 | 1 | 1 | 316 | 34.1 | 7.15 |
| | OS = Citrus sinensis | | | | | | | | |
| | GN = CISIN_1g013076mg | | | | | | | | |
| | PE = 4 SV = 1 - | | | | | | | | |
| | [A0A067EUE0_CITSI] | | | | | | | | |
| A0A067FQD2 | Uncharacterized protein | 15.28 | 3 | 2 | 6 | 18 | 445 | 48.3 | 6.64 |
| | OS = Citrus sinensis | | | | | | | | |
| | GN = CISIN_1g013317mg | | | | | | | | |
| | PE = 3 SV = 1 - | | | | | | | | |
| | [A0A067FQD2_CITSI] | | | | | | | | |
| A0A067E3Y5 | Uncharacterized protein | 19.21 | 8 | 2 | 6 | 6 | 328 | 37.4 | 7.78 |
| | OS = Citrus sinensis | | | | | | | | |
| | GN = CISIN_1g013352mg | | | | | | | | |
| | PE = 4 SV = 1 - | | | | | | | | |
| | [A0A067E3Y5_CITSI] | | | | | | | | |
| A0A067DNA9 | Uncharacterized protein | 9.84 | 8 | 3 | 4 | 4 | 437 | 46.7 | 5.11 |
| | OS = Citrus sinensis | | | | | | | | |
| | GN = CISIN_1g013743mg | | | | | | | | |
| | PE = 3 SV = 1 - | | | | | | | | |
| | [A0A067DNA9_CITSI] | | | | | | | | |
| A0A067EG97 | Uncharacterized protein | 3.29 | 3 | 1 | 1 | 1 | 395 | 44.7 | 7.78 |
| | OS = Citrus sinensis | | | | | | | | |
| | GN = CISIN_1g014082mg | | | | | | | | |
| | PE = 4 SV = 1 - | | | | | | | | |
| | [A0A067EG97_CITSI] | | | | | | | | |
| A0A067EFB3 | Uncharacterized protein | 15.96 | 6 | 5 | 5 | 5 | 376 | 41.9 | 5.44 |
| | OS = Citrus sinensis | | | | | | | | |
| | GN = CISIN_1g014332mg | | | | | | | | |
| | PE = 3 SV = 1 - | | | | | | | | |
| | [A0A067EFB3_CITSI] | | | | | | | | |
| A0A067ET10 | Uncharacterized protein | 19.78 | 8 | 2 | 5 | 6 | 359 | 39.4 | 7.36 |
| | OS = Citrus sinensis | | | | | | | | |
| | GN = CISIN_1g014463mg | | | | | | | | |
| | PE = 3 SV = 1 - | | | | | | | | |
| | [A0A067ET10_CITSI] | | | | | | | | |
| A0A067EGZ7 | Uncharacterized protein | 4.26 | 8 | 1 | 1 | 1 | 282 | 31.6 | 7.08 |
| | OS = Citrus sinensis | | | | | | | | |
| | GN = CISIN_1g0144871mg | | | | | | | | |
| | PE = 4 SV = 1 - | | | | | | | | |
| | [A0A067EGZ7_CITSI] | | | | | | | | |
| A0A067ETE8 | Uncharacterized protein | 7.36 | 3 | 2 | 2 | 2 | 394 | 45.5 | 6.93 |
| | OS = Citrus sinensis | | | | | | | | |
| | GN = CISIN_1g014576mg | | | | | | | | |
| | PE = 4 SV = 1 - | | | | | | | | |
| | [A0A067ETE8_CITSI] | | | | | | | | |
| A0A067GY21 | Uncharacterized protein | 2.41 | 2 | 1 | 1 | 1 | 374 | 41.8 | 7.99 |
| | OS = Citrus sinensis | | | | | | | | |
| | GN = CISIN_1g014743mg | | | | | | | | |
| | PE = 3 SV = 1 - | | | | | | | | |
| | [A0A067GY21_CITSI] | | | | | | | | |
| A0A067H878 | Uncharacterized protein | 3.59 | 2 | 1 | 1 | 1 | 418 | 43.3 | 5.63 |
| | OS = Citrus sinensis | | | | | | | | |
| | GN = CISIN_1g014822mg | | | | | | | | |
| | PE = 3 SV = 1 - | | | | | | | | |
| | [A0A067H878_CITSI] | | | | | | | | |
| A0A067GG31 | Uncharacterized protein | 5.60 | 3 | 1 | 1 | 1 | 339 | 35.6 | 7.24 |
| | OS = Citrus sinensis | | | | | | | | |
| | GN = CISIN_1g015003mg | | | | | | | | |
| | PE = 3 SV = 1 - | | | | | | | | |
| | [A0A067GG31_CITSI] | | | | | | | | |
| A0A067FPQ7 | Uncharacterized protein | 26.02 | 9 | 1 | 9 | 10 | 369 | 41.9 | 7.75 |
| | OS = Citrus sinensis | | | | | | | | |
| | GN = CISIN_1g015129mg | | | | | | | | |
| | PE = 3 SV = 1 - | | | | | | | | |
| | [A0A067FPQ7_CITSI] | | | | | | | | |
| A0A067ENV0 | Uncharacterized protein | 5.58 | 4 | 1 | 2 | 2 | 412 | 43.4 | 8.03 |
| | OS = Citrus sinensis | | | | | | | | |
| | GN = CISIN_1g015131mg | | | | | | | | |
| | PE = 3 SV = 1 - | | | | | | | | |
| | [A0A067ENV0_CITSI] | | | | | | | | |
| A0A067G6K1 | Uncharacterized protein | 3.42 | 7 | 1 | 1 | 1 | 263 | 30.0 | 7.52 |
| | OS = Citrus sinensis | | | | | | | | |
| | GN = CISIN_1g015465mg | | | | | | | | |
| | PE = 4 SV = 1 - | | | | | | | | |
| | [A0A067G6K1_CITSI] | | | | | | | | |
| A0A067GWQ8 | Uncharacterized protein | 4.82 | 11 | 1 | 1 | 1 | 249 | 28.4 | 6.86 |
| | OS = Citrus sinensis | | | | | | | | |
| | GN = CISIN_1g015582mg | | | | | | | | |
| | PE = 3 SV = 1 - | | | | | | | | |
| | [A0A067GWQ8_CITSI] | | | | | | | | |
| A0A067E4E7 | Uncharacterized protein | 6.98 | 9 | 2 | 2 | 2 | 315 | 34.8 | 8.46 |
| | OS = Citrus sinensis | | | | | | | | |
| | GN = CISIN_1g015695mg | | | | | | | | |
| | PE = 4 SV = 1 - | | | | | | | | |
| | [A0A067E4E7_CITSI] | | | | | | | | |
| A0A067FH37 | Uncharacterized protein | 2.76 | 2 | 1 | 1 | 1 | 398 | 45.4 | 9.11 |
| | OS = Citrus sinensis | | | | | | | | |
| | GN = CISIN_1g015925mg | | | | | | | | |
| | PE = 4 SV = 1 - | | | | | | | | |
| | [A0A067FH37_CITSI] | | | | | | | | |
| A0A067EIU1 | Uncharacterized protein | 5.26 | 4 | 2 | 2 | 2 | 380 | 41.2 | 7.33 |
| | OS = Citrus sinensis | | | | | | | | |
| | GN = CISIN_1g015935mg | | | | | | | | |
| | PE = 3 SV = 1 - | | | | | | | | |
| | [A0A067EIU1_CITSI] | | | | | | | | |
| A0A067G5B4 | Uncharacterized protein | 3.19 | 4 | 1 | 1 | 1 | 376 | 40.7 | 7.74 |
| | OS = Citrus sinensis | | | | | | | | |
| | GN = CISIN_1g016016mg | | | | | | | | |
| | PE = 3 SV = 1 - | | | | | | | | |
| | [A0A067G5B4_CITSI] | | | | | | | | |
| A0A067F5B1 | Uncharacterized protein | 12.94 | 3 | 1 | 5 | 5 | 394 | 42.8 | 9.69 |
| | OS = Citrus sinensis | | | | | | | | |
| | GN = CISIN_1g016168mg | | | | | | | | |
| | PE = 3 SV = 1 - | | | | | | | | |
| | [A0A067F5B1_CITSI] | | | | | | | | |
| A0A067E3D0 | Uncharacterized protein | 2.67 | 3 | 1 | 1 | 2 | 374 | 41.0 | 5.68 |
| | OS = Citrus sinensis | | | | | | | | |
| | GN = CISIN_1g016178mg | | | | | | | | |
| | PE = 3 SV = 1 - | | | | | | | | |
| | [A0A067E3D0_CITSI] | | | | | | | | |
| A0A067F0M0 | Uncharacterized protein | 5.84 | 2 | 2 | 2 | 2 | 394 | 43.3 | 6.76 |
| | OS = Citrus sinensis | | | | | | | | |
| | GN = CISIN_1g016183mg | | | | | | | | |
| | PE = 4 SV = 1 - | | | | | | | | |
| | [A0A067F0M0_CITSI] | | | | | | | | |
| A0A067ERC6 | Uncharacterized protein | 7.65 | 2 | 1 | 2 | 3 | 392 | 41.3 | 8.63 |
| | OS = Citrus sinensis | | | | | | | | |
| | GN = CISIN_1g016242mg | | | | | | | | |
| | PE = 4 SV = 1 - | | | | | | | | |
| | [A0A067ERC6_CITSI] | | | | | | | | |
| A0A067DZR1 | Uncharacterized protein | 9.57 | 4 | 1 | 3 | 3 | 303 | 34.6 | 10.08 |
| | OS = Citrus sinensis | | | | | | | | |
| | GN = CISIN_1g016467mg | | | | | | | | |
| | PE = 3 SV = 1 - | | | | | | | | |
| | [A0A067DZR1_CITSI] | | | | | | | | |
| A0A067G301 | Uncharacterized protein | 2.79 | 3 | 1 | 1 | 1 | 323 | 35.8 | 5.10 |
| | OS = Citrus sinensis | | | | | | | | |
| | GN = CISIN_1g016631mg | | | | | | | | |
| | PE = 4 SV = 1 - | | | | | | | | |
| | [A0A067G301_CITSI] | | | | | | | | |
| A0A067FPL5 | Uncharacterized protein | 7.65 | 4 | 2 | 2 | 2 | 340 | 39.0 | 7.34 |
| | OS = Citrus sinensis | | | | | | | | |
| | GN = CISIN_1g016653mg | | | | | | | | |
| | PE = 4 SV = 1 - | | | | | | | | |
| | [A0A067FPL5_CITSI] | | | | | | | | |
| A0A067FI16 | Uncharacterized protein | 10.98 | 5 | 3 | 3 | 3 | 255 | 27.7 | 8.16 |
| | OS = Citrus sinensis | | | | | | | | |
| | GN = CISIN_1g016978mg | | | | | | | | |
| | PE = 4 SV = 1 - | | | | | | | | |
| | [A0A067FI16_CITSI] | | | | | | | | |
| A0A067DVY8 | Uncharacterized protein | 19.26 | 8 | 3 | 7 | 7 | 379 | 41.0 | 9.73 |
| | OS = Citrus sinensis | | | | | | | | |
| | GN = CISIN_1g016996mg | | | | | | | | |
| | PE = 3 SV = 1 - | | | | | | | | |
| | [A0A067DVY8_CITSI] | | | | | | | | |
| A0A067FU47 | Uncharacterized protein | 37.14 | 13 | 1 | 11 | 14 | 377 | 41.7 | 5.49 |
| | OS = Citrus sinensis | | | | | | | | |
| | GN = CISIN_1g017089mg | | | | | | | | |
| | PE = 3 SV = 1 - | | | | | | | | |
| | [A0A067FU47_CITSI] | | | | | | | | |
| A0A067EDC1 | Uncharacterized protein | 7.49 | 3 | 2 | 2 | 2 | 374 | 42.2 | 6.21 |
| | OS = Citrus sinensis | | | | | | | | |
| | GN = CISIN_1g017216mg | | | | | | | | |
| | PE = 4 SV = 1 - | | | | | | | | |
| | [A0A067EDC1_CITSI] | | | | | | | | |
| A0A067EIG3 | Uncharacterized protein | 4.91 | 4 | 1 | 1 | 1 | 285 | 31.8 | 8.00 |
| | OS = Citrus sinensis | | | | | | | | |
| | GN = CISIN_1g017413mg | | | | | | | | |
| | PE = 3 SV = 1 - | | | | | | | | |
| | [A0A067EIG3_CITSI] | | | | | | | | |
| A0A067E5K8 | Uncharacterized protein | 4.27 | 18 | 1 | 1 | 1 | 234 | 26.3 | 6.25 |
| | OS = Citrus sinensis | | | | | | | | |
| | GN = CISIN_1g017492mg | | | | | | | | |
| | PE = 4 SV = 1 - | | | | | | | | |
| | [A0A067E5K8_CITSI] | | | | | | | | |
| A0A067GU25 | Uncharacterized protein | 8.31 | 5 | 2 | 2 | 2 | 325 | 35.0 | 7.39 |
| | OS = Citrus sinensis | | | | | | | | |
| | GN = CISIN_1g017504mg | | | | | | | | |
| | PE = 4 SV = 1 - | | | | | | | | |
| | [A0A067GU25_CITSI] | | | | | | | | |
| A0A067DWZ6 | Uncharacterized protein | 4.66 | 6 | 2 | 2 | 2 | 365 | 38.8 | 9.23 |
| | OS = Citrus sinensis | | | | | | | | |
| | GN = CISIN_1g017855mg | | | | | | | | |
| | PE = 3 SV = 1 - | | | | | | | | |
| | [A0A067DWZ6_CITSI] | | | | | | | | |
| A0A067EPQ1 | Uncharacterized protein | 2.69 | 6 | 1 | 1 | 1 | 335 | 36.3 | 5.48 |
| | OS = Citrus sinensis | | | | | | | | |
| | GN = CISIN_1g017915mg | | | | | | | | |
| | PE = 4 SV = 1 - | | | | | | | | |
| | [A0A067EPQ1_CITSI] | | | | | | | | |
| A0A067EAE1 | Uncharacterized protein | 3.45 | 3 | 1 | 1 | 1 | 261 | 29.5 | 8.41 |
| | OS = Citrus sinensis | | | | | | | | |
| | GN = CISIN_1g017985mg | | | | | | | | |
| | PE = 4 SV = 1 - | | | | | | | | |
| | [A0A067EAE1_CITSI] | | | | | | | | |
| A0A067FVZ6 | Uncharacterized protein | 3.92 | 4 | 1 | 1 | 2 | 255 | 29.2 | 5.95 |
| | OS = Citrus sinensis | | | | | | | | |
| | GN = CISIN_1g018080mg | | | | | | | | |
| | PE = 4 SV = 1 - | | | | | | | | |
| | [A0A067FVZ6_CITSI] | | | | | | | | |
| A0A067FA10 | Uncharacterized protein | 2.82 | 2 | 1 | 1 | 1 | 354 | 37.4 | 9.01 |
| | OS = Citrus sinensis | | | | | | | | |
| | GN = CISIN_1g018539mg | | | | | | | | |
| | PE = 4 SV = 1 - | | | | | | | | |
| | [A0A067FA10_CITSI] | | | | | | | | |
| A0A067E4S3 | Uncharacterized protein | 3.74 | 3 | 1 | 1 | 1 | 321 | 33.7 | 4.63 |
| | OS = Citrus sinensis | | | | | | | | |
| | GN = CISIN_1g018578mg | | | | | | | | |
| | PE = 4 SV = 1 - | | | | | | | | |
| | [A0A067E4S3_CITSI] | | | | | | | | |
| A0A067FAH2 | Uncharacterized protein | 10.89 | 5 | 2 | 2 | 2 | 248 | 27.0 | 5.47 |
| | OS = Citrus sinensis | | | | | | | | |
| | GN = CISIN_1g018696mg | | | | | | | | |
| | PE = 4 SV = 1 - | | | | | | | | |
| | [A0A067FAH2_CITSI] | | | | | | | | |
| A0A067E7B9 | Uncharacterized protein | 3.13 | 2 | 1 | 1 | 1 | 351 | 38.9 | 5.12 |
| | OS = Citrus sinensis | | | | | | | | |
| | GN = CISIN_1g018706mg | | | | | | | | |
| | PE = 4 SV = 1 - | | | | | | | | |
| | [A0A067E7B9_CITSI] | | | | | | | | |
| A0A067ENY1 | Uncharacterized protein | 4.55 | 4 | 1 | 1 | 1 | 264 | 28.8 | 6.18 |
| | OS = Citrus sinensis | | | | | | | | |
| | GN = CISIN_1g018784mg | | | | | | | | |
| | PE = 4 SV = 1 - | | | | | | | | |
| | [A0A067ENY1_CITSI] | | | | | | | | |
| A0A067E1S6 | Uncharacterized protein | 4.33 | 2 | 1 | 1 | 1 | 254 | 27.7 | 6.67 |
| | OS = Citrus sinensis | | | | | | | | |
| | GN = CISIN_1g018854mg | | | | | | | | |
| | PE = 4 SV = 1 - | | | | | | | | |
| | [A0A067E1S6_CITSI] | | | | | | | | |
| A0A067FTS6 | Uncharacterized protein | 5.22 | 8 | 1 | 1 | 1 | 249 | 27.5 | 7.08 |
| | OS = Citrus sinensis | | | | | | | | |
| | GN = CISIN_1g020099mg | | | | | | | | |
| | PE = 4 SV = 1 - | | | | | | | | |
| | [A0A067FTS6_CITSI] | | | | | | | | |
| A0A067FIY3 | Uncharacterized protein | 7.05 | 6 | 1 | 1 | 1 | 227 | 24.6 | 5.33 |
| | OS = Citrus sinensis | | | | | | | | |
| | GN = CISIN_1g020272mg | | | | | | | | |
| | PE = 4 SV = 1 - | | | | | | | | |
| | [A0A067FIY3_CITSI] | | | | | | | | |
| A0A067EQ04 | Uncharacterized protein | 4.44 | 3 | 1 | 1 | 1 | 270 | 28.4 | 5.52 |
| | OS = Citrus sinensis | | | | | | | | |
| | GN = CISIN_1g020528mg | | | | | | | | |
| | PE = 4 SV = 1 - | | | | | | | | |
| | [A0A067EQ04_CITSI] | | | | | | | | |
| A0A067GSS0 | Uncharacterized protein | 13.26 | 4 | 2 | 2 | 2 | 181 | 19.7 | 5.41 |
| | OS = Citrus sinensis | | | | | | | | |
| | GN = CISIN_1g020639mg | | | | | | | | |
| | PE = 4 SV = 1 - | | | | | | | | |
| | [A0A067GSS0_CITSI] | | | | | | | | |
| A0A067FS98 | Uncharacterized protein | 3.46 | 3 | 1 | 1 | 1 | 318 | 34.6 | 8.40 |
| | OS = Citrus sinensis | | | | | | | | |
| | GN = CISIN_1g020952mg | | | | | | | | |
| | PE = 3 SV = 1 - | | | | | | | | |
| | [A0A067FS98_CITSI] | | | | | | | | |
| A0A067EB56 | Uncharacterized protein | 6.03 | 2 | 1 | 1 | 1 | 315 | 35.3 | 6.87 |
| | OS = Citrus sinensis | | | | | | | | |
| | GN = CISIN_1g021241mg | | | | | | | | |
| | PE = 4 SV = 1 - | | | | | | | | |
| | [A0A067EB56_CITSI] | | | | | | | | |
| A0A067EA96 | Uncharacterized protein | 5.08 | 3 | 2 | 2 | 2 | 315 | 35.3 | 6.73 |
| | OS = Citrus sinensis | | | | | | | | |
| | GN = CISIN_1g021264mg | | | | | | | | |
| | PE = 4 SV = 1 - | | | | | | | | |
| | [A0A067EA96_CITSI] | | | | | | | | |
| A0A067F0C4 | Uncharacterized protein | 3.87 | 1 | 1 | 1 | 1 | 310 | 33.2 | 6.44 |
| | OS = Citrus sinensis | | | | | | | | |
| | GN = CISIN_1g021578mg | | | | | | | | |
| | PE = 3 SV = 1 - | | | | | | | | |
| | [A0A067F0C4_CITSI] | | | | | | | | |
| A0A067FSS8 | Uncharacterized protein | 4.07 | 4 | 1 | 1 | 1 | 221 | 24.8 | 8.62 |
| | OS = Citrus sinensis | | | | | | | | |
| | GN = CISIN_1g0219951mg | | | | | | | | |
| | PE = 4 SV = 1 - | | | | | | | | |
| | [A0A067FSS8_CITSI] | | | | | | | | |
| A0A067EG47 | Uncharacterized protein | 4.81 | 5 | 1 | 1 | 1 | 187 | 21.5 | 5.62 |
| | OS = Citrus sinensis | | | | | | | | |
| | GN = CISIN_1g022053mg | | | | | | | | |
| | PE = 4 SV = 1 - | | | | | | | | |
| | [A0A067EG47_CITSI] | | | | | | | | |
| A0A067EDQ9 | Uncharacterized protein | 5.00 | 5 | 1 | 1 | 1 | 220 | 23.9 | 9.10 |
| | OS = Citrus sinensis | | | | | | | | |
| | GN = CISIN_1g022158mg | | | | | | | | |
| | PE = 4 SV = 1 - | | | | | | | | |
| | [A0A067EDQ9_CITSI] | | | | | | | | |
| A0A067EW17 | Uncharacterized protein | 6.88 | 6 | 1 | 1 | 1 | 160 | 18.0 | 7.44 |
| | OS = Citrus sinensis | | | | | | | | |
| | GN = CISIN_1g022174mg | | | | | | | | |
| | PE = 4 SV = 1 - | | | | | | | | |
| | [A0A067EW17_CITSI] | | | | | | | | |
| A0A067EPB2 | Uncharacterized protein | 12.74 | 4 | 2 | 2 | 2 | 259 | 29.4 | 6.87 |
| | OS = Citrus sinensis | | | | | | | | |
| | GN = CISIN_1g022278mg | | | | | | | | |
| | PE = 4 SV = 1 - | | | | | | | | |
| | [A0A067EPB2_CITSI] | | | | | | | | |
| A0A067DPC9 | Uncharacterized protein | 6.50 | 10 | 1 | 1 | 1 | 200 | 23.2 | 9.54 |
| | OS = Citrus sinensis | | | | | | | | |
| | GN = CISIN_1g022579mg | | | | | | | | |
| | PE = 4 SV = 1 - | | | | | | | | |
| | [A0A067DPC9_CITSI] | | | | | | | | |
| A0A067H184 | Uncharacterized protein | 14.73 | 3 | 4 | 4 | 4 | 292 | 32.5 | 6.13 |
| | OS = Citrus sinensis | | | | | | | | |
| | GN = CISIN_1g022768mg | | | | | | | | |
| | PE = 3 SV = 1 - | | | | | | | | |
| | [A0A067H184_CITSI] | | | | | | | | |
| A0A067FED9 | Uncharacterized protein | 3.44 | 2 | 1 | 1 | 1 | 291 | 32.6 | 6.99 |
| | OS = Citrus sinensis | | | | | | | | |
| | GN = CISIN_1g022859mg | | | | | | | | |
| | PE = 3 SV = 1 - | | | | | | | | |
| | [A0A067FED9_CITSI] | | | | | | | | |
| A0A067FQD5 | Uncharacterized protein | 2.76 | 2 | 1 | 1 | 1 | 290 | 32.2 | 5.43 |
| | OS = Citrus sinensis | | | | | | | | |
| | GN = CISIN_1g022890mg | | | | | | | | |
| | PE = 4 SV = 1 - | | | | | | | | |
| | [A0A067FQD5_CITSI] | | | | | | | | |
| A0A067FDQ4 | Uncharacterized protein | 2.76 | 2 | 1 | 1 | 1 | 290 | 31.6 | 6.99 |
| | OS = Citrus sinensis | | | | | | | | |
| | GN = CISIN_1g022892mg | | | | | | | | |
| | PE = 3 SV = 1 - | | | | | | | | |
| | [A0A067FDQ4_CITSI] | | | | | | | | |
| A0A067GLR6 | Uncharacterized protein | 6.14 | 5 | 1 | 1 | 1 | 228 | 24.4 | 8.59 |
| | OS = Citrus sinensis | | | | | | | | |
| | GN = CISIN_1g023107mg | | | | | | | | |
| | PE = 3 SV = 1 - | | | | | | | | |
| | [A0A067GLR6_CITSI] | | | | | | | | |
| A0A067DEH3 | Uncharacterized protein | 4.01 | 3 | 1 | 1 | 1 | 274 | 30.2 | 9.20 |
| | OS = Citrus sinensis | | | | | | | | |
| | GN = CISIN_1g023231mg | | | | | | | | |
| | PE = 4 SV = 1 - | | | | | | | | |
| | [A0A067DEH3_CITSI] | | | | | | | | |
| A0A067FMZ2 | Uncharacterized protein | 8.25 | 2 | 2 | 2 | 2 | 206 | 22.4 | 9.31 |
| | OS = Citrus sinensis | | | | | | | | |
| | GN = CISIN_1g023433mg | | | | | | | | |
| | PE = 4 SV = 1 - | | | | | | | | |
| | [A0A067FMZ2_CITSI] | | | | | | | | |
| A0A067DNE8 | Uncharacterized protein | 7.86 | 4 | 2 | 2 | 2 | 280 | 30.0 | 8.57 |
| | OS = Citrus sinensis | | | | | | | | |
| | GN = CISIN_1g023572mg | | | | | | | | |
| | PE = 4 SV = 1 - | | | | | | | | |
| | [A0A067DNE8_CITSI] | | | | | | | | |
| A0A067EF17 | Uncharacterized protein | 2.89 | 1 | 1 | 1 | 1 | 277 | 31.1 | 9.44 |
| | OS = Citrus sinensis | | | | | | | | |
| | GN = CISIN_1g023804mg | | | | | | | | |
| | PE = 4 SV = 1 - | | | | | | | | |
| | [A0A067EF17_CITSI] | | | | | | | | |
| A0A067FYP7 | Uncharacterized protein | 6.93 | 1 | 1 | 1 | 1 | 274 | 29.9 | 8.46 |
| | OS = Citrus sinensis | | | | | | | | |
| | GN = CISIN_1g023969mg | | | | | | | | |
| | PE = 4 SV = 1 - | | | | | | | | |
| | [A0A067FYP7_CITSI] | | | | | | | | |
| A0A067GCF0 | Uncharacterized protein | 3.10 | 4 | 1 | 1 | 1 | 258 | 29.3 | 6.04 |
| | OS = Citrus sinensis | | | | | | | | |
| | GN = CISIN_1g024241mg | | | | | | | | |
| | PE = 4 SV = 1 - | | | | | | | | |
| | [A0A067GCF0_CITSI] | | | | | | | | |
| A0A067FJH7 | Uncharacterized protein | 11.02 | 16 | 1 | 3 | 5 | 236 | 26.4 | 4.82 |
| | OS = Citrus sinensis | | | | | | | | |
| | GN = CISIN_1g024539mg | | | | | | | | |
| | PE = 3 SV = 1 - | | | | | | | | |
| | [A0A067FJH7_CITSI] | | | | | | | | |
| A0A067DPW1 | Uncharacterized protein | 3.52 | 5 | 1 | 1 | 2 | 256 | 27.3 | 9.36 |
| | OS = Citrus sinensis | | | | | | | | |
| | GN = CISIN_1g024983mg | | | | | | | | |
| | PE = 4 SV = 1 - | | | | | | | | |
| | [A0A067DPW1_CITSI] | | | | | | | | |
| A0A067EQ37 | Uncharacterized protein | 3.91 | 3 | 1 | 1 | 2 | 256 | 27.0 | 7.71 |
| | OS = Citrus sinensis | | | | | | | | |
| | GN = CISIN_1g025197mg | | | | | | | | |
| | PE = 3 SV = 1 - | | | | | | | | |
| | [A0A067EQ37_CITSI] | | | | | | | | |
| A0A067GFJ2 | Uncharacterized protein | 4.58 | 4 | 1 | 1 | 1 | 153 | 17.0 | 7.01 |
| | OS = Citrus sinensis | | | | | | | | |
| | GN = CISIN_1g025251mg | | | | | | | | |
| | PE = 4 SV = 1 - | | | | | | | | |
| | [A0A067GFJ2_CITSI] | | | | | | | | |
| A0A067G5G5 | Uncharacterized protein | 8.07 | 6 | 2 | 2 | 2 | 223 | 23.7 | 9.23 |
| | OS = Citrus sinensis | | | | | | | | |
| | GN = CISIN_1g025388mg | | | | | | | | |
| | PE = 3 SV = 1 - | | | | | | | | |
| | [A0A067G5G5_CITSI] | | | | | | | | |
| A0A067G8M2 | Uncharacterized protein | 5.14 | 2 | 1 | 1 | 2 | 253 | 27.7 | 5.60 |
| | OS = Citrus sinensis | | | | | | | | |
| | GN = CISIN_1g025395mg | | | | | | | | |
| | PE = 4 SV = 1 - | | | | | | | | |
| | [A0A067G8M2_CITSI] | | | | | | | | |
| A0A067EGW7 | Uncharacterized protein | 3.59 | 6 | 1 | 1 | 1 | 195 | 22.6 | 8.15 |
| | OS = Citrus sinensis | | | | | | | | |
| | GN = CISIN_1g026285mg | | | | | | | | |
| | PE = 4 SV = 1 - | | | | | | | | |
| | [A0A067EGW7_CITSI] | | | | | | | | |
| A0A067GTG9 | Uncharacterized protein | 39.09 | 9 | 7 | 7 | 8 | 197 | 22.1 | 9.95 |
| | OS = Citrus sinensis | | | | | | | | |
| | GN = CISIN_1g026316mg | | | | | | | | |
| | PE = 4 SV = 1 - | | | | | | | | |
| | [A0A067GTG9_CITSI] | | | | | | | | |
| A0A067EBG5 | Uncharacterized protein | 3.40 | 2 | 1 | 1 | 1 | 235 | 26.8 | 8.54 |
| | OS = Citrus sinensis | | | | | | | | |
| | GN = CISIN_1g026695mg | | | | | | | | |
| | PE = 3 SV = 1 - | | | | | | | | |
| | [A0A067EBG5_CITSI] | | | | | | | | |
| A0A067EBT2 | Uncharacterized protein | 11.95 | 4 | 1 | 2 | 3 | 226 | 24.5 | 4.69 |
| | OS = Citrus sinensis | | | | | | | | |
| | GN = CISIN_1g027254mg | | | | | | | | |
| | PE = 3 SV = 1 - | | | | | | | | |
| | [A0A067EBT2_CITSI] | | | | | | | | |
| A0A067D779 | Uncharacterized protein | 8.24 | 6 | 1 | 1 | 1 | 170 | 18.7 | 8.51 |
| | OS = Citrus sinensis | | | | | | | | |
| | GN = CISIN_1g027418mg | | | | | | | | |
| | PE = 3 SV = 1 - | | | | | | | | |
| | [A0A067D779_CITSI] | | | | | | | | |
| A0A067GNU4 | Uncharacterized protein | 31.21 | 18 | 5 | 5 | 5 | 173 | 19.9 | 9.23 |
| | OS = Citrus sinensis | | | | | | | | |
| | GN = CISIN_1g027593mg | | | | | | | | |
| | PE = 4 SV = 1 - | | | | | | | | |
| | [A0A067GNU4_CITSI] | | | | | | | | |
| A0A067EIC3 | Uncharacterized protein | 16.95 | 7 | 3 | 3 | 5 | 177 | 20.2 | 10.42 |
| | OS = Citrus sinensis | | | | | | | | |
| | GN = CISIN_1g027696mg | | | | | | | | |
| | PE = 4 SV = 1 - | | | | | | | | |
| | [A0A067EIC3_CITSI] | | | | | | | | |
| A0A067DJ13 | Uncharacterized protein | 7.76 | 4 | 1 | 2 | 3 | 219 | 25.6 | 7.17 |
| | OS = Citrus sinensis | | | | | | | | |
| | GN = CISIN_1g027727mg | | | | | | | | |
| | PE = 4 SV = 1 - | | | | | | | | |
| | [A0A067DJ13_CITSI] | | | | | | | | |
| A0A067F5L6 | Uncharacterized protein | 10.14 | 24 | 2 | 2 | 2 | 217 | 24.1 | 6.23 |
| | OS = Citrus sinensis | | | | | | | | |
| | GN = CISIN_1g027894mg | | | | | | | | |
| | PE = 3 SV = 1 - | | | | | | | | |
| | [A0A067F5L6_CITSI] | | | | | | | | |
| A0A067GT96 | Uncharacterized protein | 18.96 | 7 | 3 | 3 | 3 | 211 | 23.2 | 7.42 |
| | OS = Citrus sinensis | | | | | | | | |
| | GN = CISIN_1g028237mg | | | | | | | | |
| | PE = 3 SV = 1 - | | | | | | | | |
| | [A0A067GT96_CITSI] | | | | | | | | |
| A0A067GV11 | Uncharacterized protein | 15.17 | 23 | 3 | 3 | 3 | 211 | 23.4 | 5.52 |
| | OS = Citrus sinensis | | | | | | | | |
| | GN = CISIN_1g028292mg | | | | | | | | |
| | PE = 3 SV = 1 - | | | | | | | | |
| | [A0A067GV11_CITSI] | | | | | | | | |
| A0A067FIF3 | Uncharacterized protein | 6.15 | 6 | 1 | 1 | 1 | 130 | 14.6 | 11.05 |
| | OS = Citrus sinensis | | | | | | | | |
| | GN = CISIN_1g028649mg | | | | | | | | |
| | PE = 3 SV = 1 - | | | | | | | | |
| | [A0A067FIF3_CITSI] | | | | | | | | |
| A0A067E1D7 | Uncharacterized protein | 32.43 | 12 | 5 | 5 | 5 | 185 | 20.9 | 5.47 |
| | OS = Citrus sinensis | | | | | | | | |
| | GN = CISIN_1g028654mg | | | | | | | | |
| | PE = 3 SV = 1 - | | | | | | | | |
| | [A0A067E1D7_CITSI] | | | | | | | | |
| A0A067E053 | Uncharacterized protein | 6.10 | 3 | 1 | 1 | 1 | 164 | 18.0 | 9.45 |
| | OS = Citrus sinensis | | | | | | | | |
| | GN = CISIN_1g028667mg | | | | | | | | |
| | PE = 4 SV = 1 - | | | | | | | | |
| | [A0A067E053_CITSI] | | | | | | | | |
| A0A067GYK4 | Uncharacterized protein | 5.39 | 4 | 1 | 1 | 1 | 167 | 19.1 | 8.72 |
| | OS = Citrus sinensis | | | | | | | | |
| | GN = CISIN_1g028737mg | | | | | | | | |
| | PE = 3 SV = 1 - | | | | | | | | |
| | [A0A067GYK4_CITSI] | | | | | | | | |
| A0A067EA65 | Uncharacterized protein | 7.39 | 4 | 1 | 1 | 1 | 176 | 19.4 | 9.74 |
| | OS = Citrus sinensis | | | | | | | | |
| | GN = CISIN_1g028761mg | | | | | | | | |
| | PE = 4 SV = 1 - | | | | | | | | |
| | [A0A067EA65_CITSI] | | | | | | | | |
| A0A067DV78 | Uncharacterized protein | 9.85 | 15 | 1 | 2 | 2 | 203 | 23.2 | 6.04 |
| | OS = Citrus sinensis | | | | | | | | |
| | GN = CISIN_1g028798mg | | | | | | | | |
| | PE = 3 SV = 1 - | | | | | | | | |
| | [A0A067DV78_CITSI] | | | | | | | | |
| A0A067FCI8 | Uncharacterized protein | 7.92 | 2 | 1 | 1 | 1 | 202 | 23.1 | 7.24 |
| | OS = Citrus sinensis | | | | | | | | |
| | GN = CISIN_1g028854mg | | | | | | | | |
| | PE = 4 SV = 1 - | | | | | | | | |
| | [A0A067FCI8_CITSI] | | | | | | | | |
| A0A067E184 | Uncharacterized protein | 5.08 | 8 | 1 | 1 | 1 | 177 | 19.4 | 7.56 |
| | OS = Citrus sinensis | | | | | | | | |
| | GN = CISIN_1g029142mg | | | | | | | | |
| | PE = 4 SV = 1 - | | | | | | | | |
| | [A0A067E184_CITSI] | | | | | | | | |
| A0A067FGS6 | Uncharacterized protein | 10.95 | 8 | 1 | 1 | 1 | 137 | 15.5 | 9.99 |
| | OS = Citrus sinensis | | | | | | | | |
| | GN = CISIN_1g029362mg | | | | | | | | |
| | PE = 4 SV = 1 - | | | | | | | | |
| | [A0A067FGS6_CITSI] | | | | | | | | |
| A0A067HGY8 | Uncharacterized protein | 14.97 | 1 | 2 | 3 | 3 | 187 | 21.0 | 10.96 |
| | OS = Citrus sinensis | | | | | | | | |
| | GN = CISIN_1g029841mg | | | | | | | | |
| | PE = 4 SV = 1 - | | | | | | | | |
| | [A0A067HGY8_CITSI] | | | | | | | | |
| A0A067EWA4 | Uncharacterized protein | 7.69 | 3 | 1 | 1 | 1 | 130 | 14.9 | 9.01 |
| | OS = Citrus sinensis | | | | | | | | |
| | GN = CISIN_1g029869mg | | | | | | | | |
| | PE = 4 SV = 1 - | | | | | | | | |
| | [A0A067EWA4_CITSI] | | | | | | | | |
| A0A067G9B7 | Uncharacterized protein | 50.98 | 13 | 6 | 6 | 9 | 153 | 17.6 | 8.40 |
| | OS = Citrus sinensis | | | | | | | | |
| | GN = CISIN_1g030193mg | | | | | | | | |
| | PE = 3 SV = 1 - | | | | | | | | |
| | [A0A067G9B7_CITSI] | | | | | | | | |
| A0A067GBP2 | Uncharacterized protein | 9.77 | 2 | 1 | 1 | 1 | 174 | 19.1 | 6.27 |
| | OS = Citrus sinensis | | | | | | | | |
| | GN = CISIN_1g030638mg | | | | | | | | |
| | PE = 4 SV = 1 - | | | | | | | | |
| | [A0A067GBP2_CITSI] | | | | | | | | |
| A0A067G4F5 | Uncharacterized protein | 33.14 | 2 | 5 | 5 | 6 | 169 | 18.8 | 6.95 |
| | OS = Citrus sinensis | | | | | | | | |
| | GN = CISIN_1g030889mg | | | | | | | | |
| | PE = 4 SV = 1 - | | | | | | | | |
| | [A0A067G4F5_CITSI] | | | | | | | | |
| A0A067G8J6 | Uncharacterized protein | 23.08 | 6 | 1 | 2 | 4 | 130 | 14.8 | 4.54 |
| | OS = Citrus sinensis | | | | | | | | |
| | GN = CISIN_1g030941mg | | | | | | | | |
| | PE = 4 SV = 1 - | | | | | | | | |
| | [A0A067G8J6_CITSI] | | | | | | | | |
| A0A067E2Q8 | Uncharacterized protein | 14.46 | 3 | 2 | 2 | 2 | 166 | 17.8 | 8.95 |
| | OS = Citrus sinensis | | | | | | | | |
| | GN = CISIN_1g031086mg | | | | | | | | |
| | PE = 3 SV = 1 - | | | | | | | | |
| | [A0A067E2Q8_CITSI] | | | | | | | | |
| A0A067DF45 | Uncharacterized protein | 13.64 | 13 | 1 | 1 | 1 | 132 | 13.3 | 7.24 |
| | OS = Citrus sinensis | | | | | | | | |
| | GN = CISIN_1g031125mg | | | | | | | | |
| | PE = 3 SV = 1 - | | | | | | | | |
| | [A0A067DF45_CITSI] | | | | | | | | |
| A0A067EZF4 | Uncharacterized protein | 5.66 | 2 | 1 | 1 | 1 | 159 | 17.3 | 4.67 |
| | OS = Citrus sinensis | | | | | | | | |
| | GN = CISIN_1g031460mg | | | | | | | | |
| | PE = 4 SV = 1 - | | | | | | | | |
| | [A0A067EZF4_CITSI] | | | | | | | | |
| A0A067FF37 | Uncharacterized protein | 8.46 | 1 | 1 | 1 | 1 | 130 | 14.8 | 10.48 |
| | OS = Citrus sinensis | | | | | | | | |
| | GN = CISIN_1g031480mg | | | | | | | | |
| | PE = 4 SV = 1 - | | | | | | | | |
| | [A0A067FF37_CITSI] | | | | | | | | |
| A0A067E4D3 | Uncharacterized protein | 8.97 | 2 | 1 | 1 | 1 | 156 | 17.5 | 7.85 |
| | OS = Citrus sinensis | | | | | | | | |
| | GN = CISIN_1g0316021mg | | | | | | | | |
| | PE = 3 SV = 1 - | | | | | | | | |
| | [A0A067E4D3_CITSI] | | | | | | | | |
| A0A067HFQ7 | Uncharacterized protein | 28.97 | 35 | 4 | 4 | 23 | 145 | 16.6 | 9.82 |
| | OS = Citrus sinensis | | | | | | | | |
| | GN = CISIN_1g031604mg | | | | | | | | |
| | PE = 4 SV = 1 - | | | | | | | | |
| | [A0A067HFQ7_CITSI] | | | | | | | | |
| A0A067DD25 | Uncharacterized protein | 9.03 | 2 | 1 | 1 | 1 | 155 | 17.5 | 9.42 |
| | OS = Citrus sinensis | | | | | | | | |
| | GN = CISIN_1g031671mg | | | | | | | | |
| | PE = 4 SV = 1 - | | | | | | | | |
| | [A0A067DD25_CITSI] | | | | | | | | |
| A0A067G4C9 | Uncharacterized protein | 11.01 | 7 | 1 | 1 | 1 | 109 | 12.9 | 8.03 |
| | OS = Citrus sinensis | | | | | | | | |
| | GN = CISIN_1g031715mg | | | | | | | | |
| | PE = 4 SV = 1 - | | | | | | | | |
| | [A0A067G4C9_CITSI] | | | | | | | | |
| A0A067GVQ3 | Uncharacterized protein | 52.54 | 5 | 5 | 5 | 5 | 118 | 13.5 | 9.70 |
| | OS = Citrus sinensis | | | | | | | | |
| | GN = CISIN_1g031787mg | | | | | | | | |
| | PE = 3 SV = 1 - | | | | | | | | |
| | [A0A067GVQ3_CITSI] | | | | | | | | |
| A0A067H0U0 | Uncharacterized protein | 12.00 | 3 | 1 | 1 | 2 | 125 | 13.7 | 9.04 |
| | OS = Citrus sinensis | | | | | | | | |
| | GN = CISIN_1g031851mg | | | | | | | | |
| | PE = 3 SV = 1 - | | | | | | | | |
| | [A0A067H0U0_CITSI] | | | | | | | | |
| A0A067E917 | Uncharacterized protein | 14.57 | 2 | 1 | 1 | 1 | 151 | 17.0 | 5.38 |
| | OS = Citrus sinensis | | | | | | | | |
| | GN = CISIN_1g031888mg | | | | | | | | |
| | PE = 4 SV = 1 - | | | | | | | | |
| | [A0A067E917_CITSI] | | | | | | | | |
| A0A067EP86 | Uncharacterized protein | 19.15 | 4 | 1 | 1 | 1 | 94 | 10.0 | 5.14 |
| | OS = Citrus sinensis | | | | | | | | |
| | GN = CISIN_1g031977mg | | | | | | | | |
| | PE = 4 SV = 1 - | | | | | | | | |
| | [A0A067EP86_CITSI] | | | | | | | | |
| A0A067G4J5 | Uncharacterized protein | 6.08 | 1 | 1 | 1 | 1 | 148 | 16.5 | 6.23 |
| | OS = Citrus sinensis | | | | | | | | |
| | GN = CISIN_1g032045mg | | | | | | | | |
| | PE = 4 SV = 1 - | | | | | | | | |
| | [A0A067G4J5_CITSI] | | | | | | | | |
| A0A067FK09 | Uncharacterized protein | 15.65 | 2 | 2 | 2 | 2 | 147 | 16.5 | 4.96 |
| | OS = Citrus sinensis | | | | | | | | |
| | GN = CISIN_1g032081mg | | | | | | | | |
| | PE = 4 SV = 1 - | | | | | | | | |
| | [A0A067FK09_CITSI] | | | | | | | | |
| A0A067GC22 | Uncharacterized protein | 20.31 | 7 | 2 | 2 | 3 | 128 | 14.2 | 10.11 |
| | OS = Citrus sinensis | | | | | | | | |
| | GN = CISIN_1g032181mg | | | | | | | | |
| | PE = 4 SV = 1 - | | | | | | | | |
| | [A0A067GC22_CITSI] | | | | | | | | |
| A0A067EAA5 | Uncharacterized protein | 17.36 | 2 | 2 | 2 | 2 | 144 | 16.3 | 7.58 |
| | OS = Citrus sinensis | | | | | | | | |
| | GN = CISIN_1g032282mg | | | | | | | | |
| | PE = 3 SV = 1 - | | | | | | | | |
| | [A0A067EAA5_CITSI] | | | | | | | | |
| A0A067GBL1 | Uncharacterized protein | 23.26 | 8 | 3 | 3 | 3 | 129 | 14.4 | 10.52 |
| | OS = Citrus sinensis | | | | | | | | |
| | GN = CISIN_1g032309mg | | | | | | | | |
| | PE = 4 SV = 1 - | | | | | | | | |
| | [A0A067GBL1_CITSI] | | | | | | | | |
| A0A067H9I6 | Uncharacterized protein | 4.93 | 2 | 1 | 1 | 1 | 142 | 16.4 | 7.66 |
| | OS = Citrus sinensis | | | | | | | | |
| | GN = CISIN_1g032384mg | | | | | | | | |
| | PE = 4 SV = 1 - | | | | | | | | |
| | [A0A067H9I6_CITSI] | | | | | | | | |
| A0A067DP37 | Uncharacterized protein | 24.60 | 3 | 3 | 3 | 3 | 126 | 13.6 | 10.70 |
| | OS = Citrus sinensis | | | | | | | | |
| | GN = CISIN_1g032476mg | | | | | | | | |
| | PE = 3 SV = 1 - | | | | | | | | |
| | [A0A067DP37_CITSI] | | | | | | | | |
| A0A067GC12 | Uncharacterized protein | 11.20 | 2 | 1 | 1 | 1 | 125 | 14.3 | 5.36 |
| | OS = Citrus sinensis | | | | | | | | |
| | GN = CISIN_1g032504mg | | | | | | | | |
| | PE = 4 SV = 1 - | | | | | | | | |
| | [A0A067GC12_CITSI] | | | | | | | | |
| A0A067GRR5 | Uncharacterized protein | 6.02 | 4 | 1 | 1 | 1 | 133 | 15.7 | 10.51 |
| | OS = Citrus sinensis | | | | | | | | |
| | GN = CISIN_1g032796mg | | | | | | | | |
| | PE = 4 SV = 1 - | | | | | | | | |
| | [A0A067GRR5_CITSI] | | | | | | | | |
| A0A067GQ55 | Uncharacterized protein | 19.19 | 3 | 1 | 1 | 1 | 99 | 10.7 | 4.78 |
| | OS = Citrus sinensis | | | | | | | | |
| | GN = CISIN_1g033259mg | | | | | | | | |
| | PE = 4 SV = 1 - | | | | | | | | |
| | [A0A067GQ55_CITSI] | | | | | | | | |
| A0A067FKD3 | Uncharacterized protein | 11.61 | 4 | 1 | 1 | 2 | 112 | 13.0 | 11.53 |
| | OS = Citrus sinensis | | | | | | | | |
| | GN = CISIN_1g033267mg | | | | | | | | |
| | PE = 3 SV = 1 - | | | | | | | | |
| | [A0A067FKD3_CITSI] | | | | | | | | |
| A0A067DTS1 | Uncharacterized protein | 9.82 | 3 | 1 | 1 | 1 | 112 | 12.2 | 9.44 |
| | OS = Citrus sinensis | | | | | | | | |
| | GN = CISIN_1g0337581mg | | | | | | | | |
| | PE = 3 SV = 1 - | | | | | | | | |
| | [A0A067DTS1_CITSI] | | | | | | | | |
| A0A067GQZ9 | Uncharacterized protein | 16.36 | 3 | 2 | 2 | 2 | 110 | 12.1 | 5.50 |
| | OS = Citrus sinensis | | | | | | | | |
| | GN = CISIN_1g033773mg | | | | | | | | |
| | PE = 4 SV = 1 - | | | | | | | | |
| | [A0A067GQZ9_CITSI] | | | | | | | | |
| A0A067FPH1 | Uncharacterized protein | 11.32 | 2 | 1 | 1 | 1 | 106 | 11.4 | 9.74 |
| | OS = Citrus sinensis | | | | | | | | |
| | GN = CISIN_1g033992mg | | | | | | | | |
| | PE = 4 SV = 1 - | | | | | | | | |
| | [A0A067FPH1_CITSI] | | | | | | | | |
| A0A067ELN9 | Uncharacterized protein | 16.33 | 2 | 1 | 1 | 1 | 98 | 10.7 | 4.83 |
| | OS = Citrus sinensis | | | | | | | | |
| | GN = CISIN_1g034299mg | | | | | | | | |
| | PE = 4 SV = 1 - | | | | | | | | |
| | [A0A067ELN9_CITSI] | | | | | | | | |
| A0A067E8D7 | Uncharacterized protein | 12.50 | 5 | 1 | 1 | 2 | 80 | 8.6 | 4.32 |
| | OS = Citrus sinensis | | | | | | | | |
| | GN = CISIN_1g034531mg | | | | | | | | |
| | PE = 3 SV = 1 - | | | | | | | | |
| | [A0A067E8D7_CITSI] | | | | | | | | |
| A0A067DLI7 | Uncharacterized protein | 19.30 | 1 | 1 | 1 | 1 | 57 | 6.4 | 4.32 |
| | OS = Citrus sinensis | | | | | | | | |
| | GN = CISIN_1g035406mg | | | | | | | | |
| | PE = 4 SV = 1 - | | | | | | | | |
| | [A0A067DLI7_CITSI] | | | | | | | | |
| A0A067FTT2 | Uncharacterized protein | 18.46 | 2 | 1 | 1 | 1 | 65 | 7.5 | 11.17 |
| | OS = Citrus sinensis | | | | | | | | |
| | GN = CISIN_1g035463mg | | | | | | | | |
| | PE = 3 SV = 1 - | | | | | | | | |
| | [A0A067FTT2_CITSI] | | | | | | | | |
| A0A067EW41 | Uncharacterized protein | 1.38 | 2 | 1 | 1 | 1 | 653 | 74.0 | 7.62 |
| | OS = Citrus sinensis | | | | | | | | |
| | GN = CISIN_1g035744mg | | | | | | | | |
| | PE = 4 SV = 1 - | | | | | | | | |
| | [A0A067EW41_CITSI] | | | | | | | | |
| A0A067G0Q0 | Uncharacterized protein | 4.09 | 2 | 1 | 1 | 1 | 318 | 34.4 | 7.05 |
| | OS = Citrus sinensis | | | | | | | | |
| | GN = CISIN_1g036252mg | | | | | | | | |
| | PE = 3 SV = 1 - | | | | | | | | |
| | [A0A067G0Q0_CITSI] | | | | | | | | |
| A0A067EDL0 | Uncharacterized protein | 4.76 | 3 | 1 | 1 | 1 | 252 | 28.7 | 5.72 |
| | OS = Citrus sinensis | | | | | | | | |
| | GN = CISIN_1g036425mg | | | | | | | | |
| | PE = 4 SV = 1 - | | | | | | | | |
| | [A0A067EDL0_CITSI] | | | | | | | | |
| A0A067ESK0 | Uncharacterized protein | 12.50 | 3 | 1 | 5 | 5 | 440 | 47.7 | 6.60 |
| | OS = Citrus sinensis | | | | | | | | |
| | GN = CISIN_1g036665mg | | | | | | | | |
| | PE = 3 SV = 1 - | | | | | | | | |
| | [A0A067ESK0_CITSI] | | | | | | | | |
| A0A067G5J8 | Uncharacterized protein | 19.64 | 1 | 1 | 1 | 1 | 56 | 6.4 | 9.91 |
| | OS = Citrus sinensis | | | | | | | | |
| | GN = CISIN_1g037282mg | | | | | | | | |
| | PE = 4 SV = 1 - | | | | | | | | |
| | [A0A067G5J8_CITSI] | | | | | | | | |
| A0A067FSX3 | Uncharacterized protein | 5.59 | 1 | 1 | 1 | 2 | 161 | 18.3 | 5.47 |
| | OS = Citrus sinensis | | | | | | | | |
| | GN = CISIN_1g037294mg | | | | | | | | |
| | PE = 3 SV = 1 - | | | | | | | | |
| | [A0A067FSX3_CITSI] | | | | | | | | |
| A0A067D9J3 | Uncharacterized protein | 1.93 | 2 | 1 | 1 | 1 | 414 | 45.0 | 8.27 |
| | OS = Citrus sinensis | | | | | | | | |
| | GN = CISIN_1g038305mg | | | | | | | | |
| | PE = 4 SV = 1 - | | | | | | | | |
| | [A0A067D9J3_CITSI] | | | | | | | | |
| A0A067EN97 | Uncharacterized protein | 30.29 | 7 | 2 | 13 | 34 | 624 | 68.3 | 5.33 |
| | OS = Citrus sinensis | | | | | | | | |
| | GN = CISIN_1g038511mg | | | | | | | | |
| | PE = 3 SV = 1 - | | | | | | | | |
| | [A0A067EN97_CITSI] | | | | | | | | |
| A0A067DHC4 | Uncharacterized protein | 15.11 | 3 | 2 | 2 | 3 | 139 | 15.9 | 9.98 |
| | OS = Citrus sinensis | | | | | | | | |
| | GN = CISIN_1g039014mg | | | | | | | | |
| | PE = 3 SV = 1 - | | | | | | | | |
| | [A0A067DHC4_CITSI] | | | | | | | | |
| A0A067EKL3 | Uncharacterized protein | 4.01 | 2 | 1 | 1 | 2 | 349 | 39.2 | 5.40 |
| | OS = Citrus sinensis | | | | | | | | |
| | GN = CISIN_1g039397mg | | | | | | | | |
| | PE = 4 SV = 1 - | | | | | | | | |
| | [A0A067EKL3_CITSI] | | | | | | | | |
| A0A067E3H3 | Uncharacterized protein | 11.58 | 1 | 1 | 1 | 1 | 95 | 11.1 | 10.10 |
| | OS = Citrus sinensis | | | | | | | | |
| | GN = CISIN_1g039578mg | | | | | | | | |
| | PE = 4 SV = 1 - | | | | | | | | |
| | [A0A067E3H3_CITSI] | | | | | | | | |
| A0A067GH18 | Uncharacterized protein | 19.18 | 2 | 4 | 4 | 12 | 245 | 28.0 | 6.73 |
| | OS = Citrus sinensis | | | | | | | | |
| | GN = CISIN_1g039930mg | | | | | | | | |
| | PE = 4 SV = 1 - | | | | | | | | |
| | [A0A067GH18_CITSI] | | | | | | | | |
| A0A067DC97 | Uncharacterized protein | 4.03 | 2 | 1 | 1 | 1 | 347 | 38.0 | 7.78 |
| | OS = Citrus sinensis | | | | | | | | |
| | GN = CISIN_1g040584mg | | | | | | | | |
| | PE = 3 SV = 1 - | | | | | | | | |
| | [A0A067DC97_CITSI] | | | | | | | | |
| A0A067EW86 | Uncharacterized protein | 5.42 | 2 | 1 | 1 | 1 | 166 | 17.1 | 9.99 |
| | OS = Citrus sinensis | | | | | | | | |
| | GN = CISIN_1g041202mg | | | | | | | | |
| | PE = 4 SV = 1 - | | | | | | | | |
| | [A0A067EW86_CITSI] | | | | | | | | |
| A0A067D4F6 | Uncharacterized protein | 13.35 | 2 | 1 | 5 | 16 | 412 | 45.2 | 5.39 |
| | OS = Citrus sinensis | | | | | | | | |
| | GN = CISIN_1g041209mg | | | | | | | | |
| | PE = 3 SV = 1 - | | | | | | | | |
| | [A0A067D4F6_CITSI] | | | | | | | | |
| A0A067DFL9 | Uncharacterized protein | 5.56 | 8 | 1 | 1 | 1 | 180 | 20.1 | 8.50 |
| | OS = Citrus sinensis | | | | | | | | |
| | GN = CISIN_1g041355mg | | | | | | | | |
| | PE = 4 SV = 1 - | | | | | | | | |
| | [A0A067DFL9_CITSI] | | | | | | | | |
| A0A067DSC7 | Uncharacterized protein | 5.32 | 1 | 1 | 1 | 1 | 188 | 20.9 | 5.17 |
| | OS = Citrus sinensis | | | | | | | | |
| | GN = CISIN_1g041772mg | | | | | | | | |
| | PE = 4 SV = 1 - | | | | | | | | |
| | [A0A067DSC7_CITSI] | | | | | | | | |
| A0A067GCM1 | Uncharacterized protein | 2.10 | 3 | 1 | 1 | 1 | 523 | 57.6 | 6.01 |
| | OS = Citrus sinensis | | | | | | | | |
| | GN = CISIN_1g042402mg | | | | | | | | |
| | PE = 4 SV = 1 - | | | | | | | | |
| | [A0A067GCM1_CITSI] | | | | | | | | |
| A0A067EJK1 | Uncharacterized protein | 25.08 | 7 | 1 | 11 | 31 | 650 | 71.0 | 5.59 |
| | OS = Citrus sinensis | | | | | | | | |
| | GN = CISIN_1g043296mg | | | | | | | | |
| | PE = 3 SV = 1 - | | | | | | | | |
| | [A0A067EJK1_CITSI] | | | | | | | | |
| A0A067EWH0 | Uncharacterized protein | 6.61 | 2 | 1 | 1 | 1 | 121 | 12.8 | 8.69 |
| | OS = Citrus sinensis | | | | | | | | |
| | GN = CISIN_1g043331mg | | | | | | | | |
| | PE = 4 SV = 1 - | | | | | | | | |
| | [A0A067EWH0_CITSI] | | | | | | | | |
| A0A067EVW7 | Uncharacterized protein | 7.90 | 1 | 1 | 2 | 3 | 443 | 48.9 | 5.54 |
| | OS = Citrus sinensis | | | | | | | | |
| | GN = CISIN_1g043979mg | | | | | | | | |
| | PE = 3 SV = 1 - | | | | | | | | |
| | [A0A067EVW7_CITSI] | | | | | | | | |
| A0A067G593 | Uncharacterized protein | 16.24 | 2 | 2 | 2 | 3 | 197 | 21.2 | 6.87 |
| | OS = Citrus sinensis | | | | | | | | |
| | GN = CISIN_1g044973mg | | | | | | | | |
| | PE = 4 SV = 1 - | | | | | | | | |
| | [A0A067G593_CITSI] | | | | | | | | |
| A0A067F3X5 | Uncharacterized protein | 5.19 | 2 | 2 | 2 | 2 | 578 | 63.3 | 6.86 |
| | OS = Citrus sinensis | | | | | | | | |
| | GN = CISIN_1g045794mg | | | | | | | | |
| | PE = 3 SV = 1 - | | | | | | | | |
| | [A0A067F3X5_CITSI] | | | | | | | | |
| A0A067EQ35 | Uncharacterized protein | 6.64 | 2 | 1 | 1 | 2 | 256 | 26.4 | 5.34 |
| | OS = Citrus sinensis | | | | | | | | |
| | GN = CISIN_1g046001mg | | | | | | | | |
| | PE = 4 SV = 1 - | | | | | | | | |
| | [A0A067EQ35_CITSI] | | | | | | | | |
| A0A067EHH8 | Uncharacterized protein | 12.67 | 2 | 9 | 9 | 10 | 900 | 100.0 | 6.02 |
| | OS = Citrus sinensis | | | | | | | | |
| | GN = CISIN_1g046258mg | | | | | | | | |
| | PE = 3 SV = 1 - | | | | | | | | |
| | [A0A067EHH8_CITSI] | | | | | | | | |
| A0A067FJ03 | Uncharacterized protein | 6.87 | 2 | 1 | 1 | 1 | 131 | 14.8 | 10.35 |
| | OS = Citrus sinensis | | | | | | | | |
| | GN = CISIN_1g046599mg | | | | | | | | |
| | PE = 4 SV = 1 - | | | | | | | | |
| | [A0A067FJ03_CITSI] | | | | | | | | |
| A0A067D278 | Uncharacterized protein | 5.42 | 2 | 1 | 1 | 1 | 166 | 18.1 | 9.99 |
| | OS = Citrus sinensis | | | | | | | | |
| | GN = CISIN_1g047028mg | | | | | | | | |
| | PE = 4 SV = 1 - | | | | | | | | |
| | [A0A067D278_CITSI] | | | | | | | | |
| A0A067D4M8 | Uncharacterized protein | 7.55 | 3 | 1 | 1 | 1 | 106 | 12.4 | 10.21 |
| | OS = Citrus sinensis | | | | | | | | |
| | GN = CISIN_1g047565mg | | | | | | | | |
| | PE = 4 SV = 1 - | | | | | | | | |
| | [A0A067D4M8_CITSI] | | | | | | | | |
| A0A067GDZ7 | Uncharacterized protein | 4.55 | 2 | 1 | 1 | 1 | 154 | 17.9 | 10.48 |
| | OS = Citrus sinensis | | | | | | | | |
| | GN = CISIN_1g048091mg | | | | | | | | |
| | PE = 4 SV = 1 - | | | | | | | | |
| | [A0A067GDZ7_CITSI] | | | | | | | | |
| A0A067F699 | Uncharacterized protein | 4.29 | 2 | 1 | 1 | 3 | 303 | 33.4 | 7.72 |
| | OS = Citrus sinensis | | | | | | | | |
| | GN = CISIN_1g048118mg | | | | | | | | |
| | PE = 4 SV = 1 - | | | | | | | | |
| | [A0A067F699_CITSI] | | | | | | | | |
| Q9MB48 | Vacuolar H+-ATPase B | 6.85 | 5 | 2 | 2 | 2 | 365 | 40.3 | 4.83 |
| | subunit (Fragment) | | | | | | | | |
| | OS = Citrus unshiu | | | | | | | | |
| | GN = Cit-VATP B-1 PE = 3 | | | | | | | | |
| | SV = 1 - [Q9MB48_CITUN] | | | | | | | | |
| Q38758 | V-ATPase 66 kDa subunit | 3.57 | 5 | 1 | 1 | 1 | 336 | 36.0 | 5.80 |
| | (Fragment) OS = Acer | | | | | | | | |
| | pseudoplatanus PE = 2 SV = 1 - | | | | | | | | |
| | [Q38758_ACEPS] | | | | | | | | |
| Q8L5L6 | Vicilin-like protein | 33.77 | 2 | 14 | 14 | 61 | 536 | 61.6 | 5.85 |
| | (Fragment) OS = Anacardium | | | | | | | | |
| | occidentale GN = Ana o | | | | | | | | |
| | 1.0102 PE = 2 SV = 1 - | | | | | | | | |
| | [Q8L5L6_ANAOC] | | | | | | | | |

A Total of 843 new found proteins in Edamame (young soy) and their sequence.

| | | | | # | | | | | |
|---|---|---|---|---|---|---|---|---|---|
| | | | # | Unique | # | # | # | MW | calc. |
| Accession | Description | Coverage | Proteins | Peptides | Peptides | PSMs | AAs | [kDa] | pI |
| Q96453 | 14-3-3-like protein D | 21.46 | 9 | 1 | 4 | 4 | 261 | 29.5 | 4.83 |
| | OS = Glycine max GN = GF14D | | | | | | | | |
| | PE = 2 SV = 1 - [1433D_SOYBN] | | | | | | | | |
| P04794 | 17.5 kDa class I heat shock | 35.71 | 10 | 1 | 6 | 6 | 154 | 17.5 | 6.37 |
| | protein OS = Glycine max | | | | | | | | |
| | GN = HSP17.5-E PE = 3 SV = 1 - | | | | | | | | |
| | [HSP14_SOYBN] | | | | | | | | |
| Q01417 | 18 kDa seed maturation protein | 21.97 | 1 | 2 | 3 | 4 | 173 | 17.6 | 9.58 |
| | OS = Glycine max GN = GMPM1 | | | | | | | | |
| | PE = 2 SV = 1 - [PM1_SOYBN] | | | | | | | | |
| P05478 | 18.5 kDa class I heat shock | 31.06 | 3 | 1 | 5 | 5 | 161 | 18.5 | 6.09 |
| | protein OS = Glycine max | | | | | | | | |
| | GN = HSP18.5-C PE = 3 SV = 1 - | | | | | | | | |
| | [HSP16_SOYBN] | | | | | | | | |
| Q8W0V9 | 24 kDa protein SC24 | 22.37 | 3 | 4 | 4 | 4 | 219 | 24.6 | 9.11 |
| | OS = Glycine max PE = 2 SV = 1 - | | | | | | | | |
| | [Q8W0V9_SOYBN] | | | | | | | | |
| P19594 | 2S albumin OS = Glycine max | 51.27 | 3 | 8 | 8 | 63 | 158 | 18.4 | 5.34 |
| | PE = 1 SV = 2 - [2SS_SOYBN] | | | | | | | | |
| O64458 | 34 kDa maturing seed protein | 18.47 | 3 | 6 | 6 | 47 | 379 | 42.7 | 5.99 |
| | OS = Glycine max GN = P34 | | | | | | | | |
| | PE = 2 SV = 1 - | | | | | | | | |
| | [O64458_SOYBN] | | | | | | | | |
| C6SW79 | 40S ribosomal protein S12 | 31.43 | 8 | 3 | 3 | 6 | 140 | 14.8 | 5.49 |
| | OS = Glycine max PE = 2 SV = 1 - | | | | | | | | |
| | [C6SW79_SOYBN] | | | | | | | | |
| C6T6P9 | 40S ribosomal protein S21 | 32.10 | 3 | 2 | 2 | 2 | 81 | 9.1 | 6.77 |
| | OS = Glycine max PE = 2 SV = 1 - | | | | | | | | |
| | [C6T6P9_SOYBN] | | | | | | | | |
| C6SVP0 | 40S ribosomal protein S24 | 18.98 | 3 | 2 | 2 | 3 | 137 | 15.8 | 10.71 |
| | OS = Glycine max PE = 2 SV = 1 - | | | | | | | | |
| | [C6SVP0_SOYBN] | | | | | | | | |
| C6TAW2 | 40S ribosomal protein S24 | 25.55 | 2 | 3 | 3 | 5 | 137 | 15.8 | 10.64 |
| | OS = Glycine max PE = 2 SV = 1 - | | | | | | | | |
| | [C6TAW2_SOYBN] | | | | | | | | |
| Q6VEV1 | 40S ribosomal protein S25 | 32.98 | 7 | 3 | 3 | 4 | 94 | 10.5 | 10.76 |
| | (Fragment) OS = Glycine max | | | | | | | | |
| | PE = 2 SV = 1 - | | | | | | | | |
| | [Q6VEV1_SOYBN] | | | | | | | | |
| C6SYW6 | 40S ribosomal protein S27 | 19.77 | 1 | 1 | 1 | 1 | 86 | 9.6 | 8.73 |
| | OS = Glycine max PE = 2 SV = 1 - | | | | | | | | |
| | [C6SYW6_SOYBN] | | | | | | | | |
| I1JD07 | 40S ribosomal protein S27 | 19.77 | 2 | 1 | 1 | 2 | 86 | 9.6 | 8.73 |
| | OS = Glycine max PE = 3 SV = 1 - | | | | | | | | |
| | [I1JD07_SOYBN] | | | | | | | | |
| C6T5T0 | 40S ribosomal protein S3a | 33.48 | 6 | 2 | 7 | 9 | 227 | 26.0 | 10.18 |
| | OS = Glycine max PE = 2 SV = 1 - | | | | | | | | |
| | [C6T5T0_SOYBN] | | | | | | | | |
| C6TLT3 | 40S ribosomal protein S3a | 24.90 | 1 | 1 | 6 | 6 | 261 | 29.7 | 9.80 |
| | OS = Glycine max PE = 2 SV = 1 - | | | | | | | | |
| | [C6TLT3_SOYBN] | | | | | | | | |
| I1NGJ0 | 40S ribosomal protein S3a | 36.91 | 4 | 2 | 8 | 10 | 233 | 26.8 | 10.15 |
| | OS = Glycine max PE = 3 SV = 1 - | | | | | | | | |
| | [I1NGJ0_SOYBN] | | | | | | | | |
| C6TJG6 | 40S ribosomal protein S4 | 46.97 | 9 | 2 | 13 | 25 | 264 | 29.8 | 10.20 |
| | OS = Glycine max PE = 2 SV = 1 - | | | | | | | | |
| | [C6TJG9_SOYBN] | | | | | | | | |
| Q8LJW0 | 40S ribosomal protein S4 | 43.18 | 5 | 1 | 12 | 20 | 264 | 29.9 | 10.26 |
| | OS = Glycine max PE = 2 SV = 1 - | | | | | | | | |
| | [Q8LJW0_SOYBN] | | | | | | | | |
| B2BF98 | 40S ribosomal protein S6 | 25.10 | 4 | 2 | 5 | 6 | 247 | 28.0 | 10.71 |
| | OS = Glycine max PE = 3 SV = 1 - | | | | | | | | |
| | [B2BF98_SOYBN] | | | | | | | | |
| I1KYY4 | 40S ribosomal protein S6 | 34.27 | 10 | 1 | 7 | 8 | 248 | 28.2 | 10.71 |
| | OS = Glycine max PE = 3 SV = 1 - | | | | | | | | |
| | [I1KYY4_SOYBN] | | | | | | | | |
| C6T968 | 40S ribosomal protein S8 | 33.64 | 3 | 3 | 6 | 6 | 217 | 24.7 | 10.42 |
| | OS = Glycine max PE = 2 SV = 1 - | | | | | | | | |
| | [C6T968_SOYBN] | | | | | | | | |
| I1JM70 | 40S ribosomal protein S8 | 36.73 | 3 | 2 | 5 | 7 | 196 | 22.0 | 10.52 |
| | OS = Glycine max PE = 3 SV = 1 - | | | | | | | | |
| | [I1JM70_SOYBN] | | | | | | | | |
| I1N0F1 | 40S ribosomal protein S8 | 25.57 | 2 | 2 | 4 | 7 | 219 | 24.7 | 10.40 |
| | OS = Glycine max PE = 3 SV = 2 - | | | | | | | | |
| | [I1N0F1_SOYBN] | | | | | | | | |
| C6TG12 | 40S ribosomal protein SA | 21.22 | 4 | 3 | 5 | 5 | 311 | 34.1 | 5.38 |
| | OS = Glycine max PE = 2 SV = 1 - | | | | | | | | |
| | [C6TG12_SOYBN] | | | | | | | | |
| O22518 | 40S ribosomal protein SA | 23.55 | 3 | 4 | 6 | 8 | 310 | 33.9 | 5.26 |
| | OS = Glycine max PE = 2 SV = 1 - | | | | | | | | |
| | [RSSA_SOYBN] | | | | | | | | |
| C6SZE4 | 4-hydroxy-4-methyl-2-oxoglutarate | 13.25 | 1 | 1 | 1 | 1 | 166 | 17.7 | 6.79 |
| | aldolase OS = Glycine max | | | | | | | | |
| | PE = 2 SV = 1 - | | | | | | | | |
| | [C6SZE4_SOYBN] | | | | | | | | |
| C6TFT6 | 4-hydroxy-4-methyl-2-oxoglutarate | 16.22 | 2 | 2 | 2 | 2 | 148 | 15.8 | 6.54 |
| | aldolase OS = Glycine max | | | | | | | | |
| | PE = 2 SV = 1 - | | | | | | | | |
| | [C6TFT6_SOYBN] | | | | | | | | |
| I1M6Z5 | 4-hydroxyphenylpyruvate | 2.70 | 5 | 1 | 1 | 1 | 408 | 44.7 | 6.05 |
| | dioxygenase OS = Glycine max | | | | | | | | |
| | PE = 3 SV = 1 - | | | | | | | | |
| | [I1M6Z5_SOYBN] | | | | | | | | |
| Q42800 | 4-hydroxy-tetrahydrodipicolinate | 5.42 | 4 | 2 | 2 | 2 | 332 | 36.4 | 6.32 |
| | synthase, chloroplastic | | | | | | | | |
| | OS = Glycine max GN = DHPS1 | | | | | | | | |
| | PE = 2 SV = 1 - [DAPA_SOYBN] | | | | | | | | |
| Q39801 | 51 kDa seed maturation protein | 35.10 | 1 | 9 | 13 | 13 | 473 | 51.0 | 7.11 |
| | OS = Glycine max PE = 2 SV = 1 - | | | | | | | | |
| | [Q39801_SOYBN] | | | | | | | | |
| C6THP8 | 6,7-dimethyl-8-ribityllumazine | 3.72 | 2 | 1 | 1 | 1 | 188 | 20.0 | 7.96 |
| | synthase OS = Glycine max | | | | | | | | |
| | PE = 2 SV = 1 - | | | | | | | | |
| | [C6THP8_SOYBN] | | | | | | | | |
| P50346 | 60S acidic ribosomal protein P0 | 27.50 | 3 | 2 | 5 | 5 | 320 | 34.1 | 5.25 |
| | OS = Glycine max PE = 2 SV = 1 - | | | | | | | | |
| | [RLA0_SOYBN] | | | | | | | | |
| C6TL61 | 60S ribosomal protein L13 | 30.43 | 7 | 5 | 5 | 5 | 207 | 23.7 | 10.93 |
| | OS = Glycine max PE = 2 SV = 1 - | | | | | | | | |
| | [C6TL61_SOYBN] | | | | | | | | |
| C6SYA5 | 60S ribosomal protein L18a | 30.90 | 7 | 1 | 6 | 7 | 178 | 21.3 | 10.42 |
| | OS = Glycine max PE = 2 SV = 1 - | | | | | | | | |
| | [C6SYA5_SOYBN] | | | | | | | | |
| C6T0E8 | 60S ribosomal protein L18a | 31.46 | 7 | 1 | 6 | 7 | 178 | 21.3 | 10.43 |
| | OS = Glycine max PE = 2 SV = 1 - | | | | | | | | |
| | [C6T0E8_SOYBN] | | | | | | | | |
| I1M6Z0 | 60S ribosomal protein L18a | 27.53 | 8 | 1 | 5 | 6 | 178 | 21.3 | 10.42 |
| | OS = Glycine max PE = 3 SV = 1 - | | | | | | | | |
| | [I1M6Z0_SOYBN] | | | | | | | | |
| C6T3R4 | 60S ribosomal protein L27 | 22.22 | 5 | 1 | 3 | 3 | 135 | 15.6 | 10.42 |
| | OS = Glycine max PE = 2 SV = 1 - | | | | | | | | |
| | [C6T3R4_SOYBN] | | | | | | | | |
| I1KRI7 | 60S ribosomal protein L27 | 22.22 | 5 | 1 | 3 | 3 | 135 | 15.7 | 10.42 |
| | OS = Glycine max PE = 3 SV = 1 - | | | | | | | | |
| | [I1KRI7_SOYBN] | | | | | | | | |
| C6TK30 | 60S ribosomal protein L36 | 30.00 | 5 | 3 | 3 | 3 | 110 | 12.4 | 11.31 |
| | OS = Glycine max PE = 2 SV = 1 - | | | | | | | | |
| | [C6TK30_SOYBN] | | | | | | | | |
| I1N4U1 | 6-phosphogluconate | 13.17 | 6 | 6 | 6 | 6 | 486 | 53.6 | 5.90 |
| | dehydrogenase, decarboxylating | | | | | | | | |
| | OS = Glycine max PE = 3 SV = 1 - | | | | | | | | |
| | [I1N4U1_SOYBN] | | | | | | | | |
| O23960 | Acetyl-CoA carboxylase | 4.45 | 3 | 2 | 2 | 2 | 539 | 58.9 | 7.50 |
| | OS = Glycine max GN = accC-2 | | | | | | | | |
| | PE = 2 SV = 1 - | | | | | | | | |
| | [O23960_SOYBN] | | | | | | | | |
| I1KH72 | Acetyltransferase component of | 14.65 | 2 | 2 | 6 | 6 | 546 | 59.3 | 7.91 |
| | pyruvate dehydrogenase | | | | | | | | |
| | complex OS = Glycine max | | | | | | | | |
| | PE = 3 SV = 1 - | | | | | | | | |
| | [I1KH72_SOYBN] | | | | | | | | |
| I1MJX0 | Acetyltransferase component of | 11.88 | 1 | 1 | 5 | 5 | 547 | 59.6 | 7.69 |
| | pyruvate dehydrogenase | | | | | | | | |
| | complex OS = Glycine max | | | | | | | | |
| | PE = 3 SV = 1 - | | | | | | | | |
| | [I1MJX0_SOYBN] | | | | | | | | |
| I1J8H1 | Aconitate hydratase | 11.32 | 6 | 5 | 8 | 8 | 901 | 98.5 | 5.86 |
| | OS = Glycine max PE = 3 SV = 1 - | | | | | | | | |
| | [I1J8H1_SOYBN] | | | | | | | | |
| I1LU53 | Aconitate hydratase | 9.65 | 8 | 6 | 9 | 9 | 984 | 107.2 | 7.99 |
| | OS = Glycine max PE = 3 SV = 1 - | | | | | | | | |
| | [I1LU53_SOYBN] | | | | | | | | |
| C6SVF1 | Actin depolymerizing factor 1 | 18.71 | 8 | 2 | 2 | 2 | 139 | 16.0 | 6.57 |
| | OS = Glycine max GN = ADF1 | | | | | | | | |
| | PE = 2 SV = 1 - | | | | | | | | |
| | [C6SVF1_SOYBN] | | | | | | | | |
| P02580 | Actin-3 OS = Glycine max | 30.85 | 6 | 3 | 10 | 12 | 376 | 41.6 | 5.40 |
| | GN = SAC3 PE = 3 SV = 2 - | | | | | | | | |
| | [ACT3_SOYBN] | | | | | | | | |
| C6SVA9 | Acyl carrier protein | 10.16 | 3 | 1 | 1 | 1 | 128 | 14.3 | 5.57 |
| | OS = Glycine max PE = 2 SV = 1 - | | | | | | | | |
| | [C6SVA9_SOYBN] | | | | | | | | |
| I1M1B6 | Acyl carrier protein | 10.79 | 1 | 1 | 1 | 1 | 139 | 14.9 | 5.31 |
| | OS = Glycine max PE = 3 SV = 1 - | | | | | | | | |
| | [I1M1B6_SOYBN] | | | | | | | | |
| B8XJY3 | Acyl-[acyl-carrier-protein] | 11.51 | 15 | 5 | 5 | 5 | 391 | 44.9 | 6.33 |
| | desaturase OS = Glycine max | | | | | | | | |
| | GN = SAD1 PE = 2 SV = 1 - | | | | | | | | |
| | [B8XJY3_SOYBN] | | | | | | | | |
| Q945U3 | Acyl-coenzyme A oxidase | 3.31 | 1 | 2 | 2 | 2 | 665 | 74.5 | 8.10 |
| | OS = Glycine max GN = ACX1; 2 | | | | | | | | |
| | PE = 2 SV = 1 - | | | | | | | | |
| | [Q945U3_SOYBN] | | | | | | | | |
| F8SMB9 | Adenosylhomocysteinase | 22.06 | 4 | 2 | 10 | 11 | 485 | 53.2 | 6.01 |
| | OS = Glycine max PE = 2 SV = 1 - | | | | | | | | |
| | [F8SMB9_SOYBN] | | | | | | | | |
| I1K3S5 | Adenosylhomocysteinase | 23.30 | 4 | 2 | 10 | 11 | 485 | 53.2 | 5.88 |
| | OS = Glycine max PE = 3 SV = 1 - | | | | | | | | |
| | [I1K3S5_SOYBN] | | | | | | | | |
| I1JNF2 | Adenylosuccinate synthetase, | 8.02 | 2 | 3 | 3 | 4 | 486 | 52.9 | 7.61 |
| | chloroplastic OS = Glycine max | | | | | | | | |
| | GN = PURA PE = 3 SV = 1 - | | | | | | | | |
| | [I1JNF2_SOYBN] | | | | | | | | |
| Q06763 | ADR11 protein (Fragment) | 6.62 | 3 | 1 | 1 | 1 | 151 | 15.5 | 8.56 |
| | OS = Glycine max GN = ADR11 | | | | | | | | |
| | PE = 2 SV = 1 - | | | | | | | | |
| | [Q06763_SOYBN] | | | | | | | | |
| I1LIT8 | Alanine--tRNA ligase | 8.91 | 2 | 7 | 7 | 7 | 999 | 109.6 | 5.97 |
| | OS = Glycine max PE = 3 SV = 2 - | | | | | | | | |
| | [I1LIT8_SOYBN] | | | | | | | | |
| Q8LJR2 | Alcohol dehydrogenase 1 | 35.42 | 2 | 6 | 11 | 27 | 367 | 40.0 | 6.61 |
| | (Fragment) OS = Glycine max | | | | | | | | |
| | PE = 2 SV = 1 - | | | | | | | | |
| | [Q8LJR2_SOYBN] | | | | | | | | |
| I1KJ60 | Aldose 1-epimerase | 5.60 | 5 | 2 | 2 | 2 | 339 | 37.0 | 6.46 |
| | OS = Glycine max PE = 3 SV = 1 - | | | | | | | | |
| | [I1KJ60_SOYBN] | | | | | | | | |
| I1N4B8 | Aldose 1-epimerase | 15.09 | 3 | 5 | 5 | 5 | 371 | 40.7 | 9.38 |
| | OS = Glycine max PE = 3 SV = 1 - | | | | | | | | |
| | [I1N4B8_SOYBN] | | | | | | | | |
| F6KBT6 | Allene oxide cyclase 6 | 8.63 | 3 | 2 | 2 | 2 | 253 | 28.3 | 9.06 |
| | OS = Glycine max GN = AOC6 | | | | | | | | |
| | PE = 2 SV = 1 - | | | | | | | | |
| | [F6KBT6_SOYBN] | | | | | | | | |
| O22120 | Alpha subunit of beta | 57.83 | 10 | 7 | 31 | 220 | 543 | 63.1 | 4.96 |
| | conglycinin (Fragment) | | | | | | | | |
| | OS = Glycine max PE = 2 SV = 2 - | | | | | | | | |
| | [O22120_SOYBN] | | | | | | | | |
| I1LWR4 | Alpha-1,4 glucan phosphorylase | 8.90 | 4 | 1 | 6 | 8 | 978 | 110.0 | 5.72 |
| | OS = Glycine max PE = 3 SV = 1 - | | | | | | | | |
| | [I1LWR4_SOYBN] | | | | | | | | |
| I1N6A5 | Alpha-1,4 glucan phosphorylase | 12.54 | 4 | 3 | 8 | 10 | 981 | 110.3 | 5.47 |
| | OS = Glycine max PE = 3 SV = 1 - | | | | | | | | |
| | [I1N6A5_SOYBN] | | | | | | | | |
| I1L6Y9 | Alpha-galactosidase | 13.66 | 2 | 2 | 5 | 7 | 410 | 45.6 | 7.15 |
| | OS = Glycine max PE = 3 SV = 1 - | | | | | | | | |
| | [I1L6Y9_SOYBN] | | | | | | | | |
| K7MTV2 | Alpha-galactosidase | 12.54 | 7 | 1 | 4 | 6 | 335 | 37.3 | 6.64 |
| | OS = Glycine max PE = 3 SV = 1 - | | | | | | | | |
| | [K7MTV2_SOYBN] | | | | | | | | |
| I1ML97 | Alpha-mannosidase | 9.32 | 5 | 6 | 8 | 8 | 1030 | 117.0 | 5.91 |
| | OS = Glycine max PE = 3 SV = 1 - | | | | | | | | |
| | [I1ML97_SOYBN] | | | | | | | | |
| I1JXQ5 | Alpha-mannosidase | 3.66 | 2 | 1 | 3 | 3 | 1011 | 114.2 | 6.70 |
| | OS = Glycine max PE = 3 SV = 2 - | | | | | | | | |
| | [I1JXQ5_SOYBN] | | | | | | | | |
| K7N0I2 | Aminoacylase-1 OS = Glycine | 2.02 | 2 | 1 | 1 | 1 | 446 | 49.7 | 6.90 |
| | max PE = 3 SV = 1 - | | | | | | | | |
| | [K7N0I2_SOYBN] | | | | | | | | |
| C6TF02 | Aminomethyltransferase | 2.70 | 6 | 1 | 1 | 1 | 407 | 44.2 | 8.75 |
| | OS = Glycine max PE = 2 SV = 1 - | | | | | | | | |
| | [C6TF02_SOYBN] | | | | | | | | |
| C6T7M2 | Annexin OS = Glycine max | 49.36 | 13 | 5 | 15 | 17 | 312 | 35.8 | 7.91 |
| | PE = 2 SV = 1 - | | | | | | | | |
| | [C6T7M2_SOYBN] | | | | | | | | |
| G3E7M9 | Annexin OS = Glycine max | 30.38 | 6 | 9 | 9 | 10 | 316 | 35.9 | 6.96 |
| | PE = 2 SV = 1 - | | | | | | | | |
| | [G3E7M9_SOYBN] | | | | | | | | |
| I1L416 | Annexin OS = Glycine max | 5.79 | 4 | 1 | 1 | 1 | 190 | 21.3 | 8.95 |
| | PE = 3 SV = 1 - | | | | | | | | |
| | [I1L416_SOYBN] | | | | | | | | |
| I1LLC1 | Annexin OS = Glycine max | 46.03 | 5 | 1 | 11 | 16 | 239 | 27.5 | 7.30 |
| | PE = 3 SV = 1 - | | | | | | | | |
| | [I1LLC1_SOYBN] | | | | | | | | |
| Q39843 | Ascorbate peroxidase 2 | 31.20 | 7 | 5 | 5 | 7 | 250 | 27.1 | 5.99 |
| | OS = Glycine max GN = APx2 | | | | | | | | |
| | PE = 2 SV = 1 - | | | | | | | | |
| | [Q39843_SOYBN] | | | | | | | | |
| Q7G1G6 | Aspartate aminotransferase | 28.64 | 3 | 2 | 10 | 11 | 419 | 45.6 | 7.88 |
| | OS = Glycine max GN = AAT | | | | | | | | |
| | PE = 2 SV = 1 - | | | | | | | | |
| | [Q7G1G6_SOYBN] | | | | | | | | |
| Q42803 | Aspartate aminotransferase | 2.58 | 1 | 1 | 1 | 1 | 427 | 47.6 | 8.54 |
| | OS = Glycine max PE = 2 SV = 1 - | | | | | | | | |
| | [Q42803_SOYBN] | | | | | | | | |
| I1JUS6 | Aspartate aminotransferase | 29.09 | 3 | 3 | 11 | 12 | 416 | 45.3 | 7.94 |
| | OS = Glycine max PE = 3 SV = 1 - | | | | | | | | |
| | [I1JUS6_SOYBN] | | | | | | | | |
| I1M9F7 | Aspartate aminotransferase | 19.87 | 6 | 6 | 7 | 7 | 463 | 50.6 | 7.90 |
| | OS = Glycine max PE = 3 SV = 1 - | | | | | | | | |
| | [I1M9F7_SOYBN] | | | | | | | | |
| D2DJQ5 | Aspartate-semialdehyde | 7.56 | 3 | 2 | 2 | 2 | 344 | 37.6 | 6.33 |
| | dehydrogenase (Fragment) | | | | | | | | |
| | OS = Glycine max PE = 2 SV = 1 - | | | | | | | | |
| | [D2DJQ5_SOYBN] | | | | | | | | |
| K7LWI4 | ATP synthase subunit alpha | 4.51 | 3 | 1 | 2 | 2 | 510 | 55.7 | 5.21 |
| | OS = Glycine max PE = 3 SV = 1 - | | | | | | | | |
| | [K7LWI4_SOYBN] | | | | | | | | |
| Q01915 | ATP synthase subunit alpha, | 23.23 | 5 | 10 | 11 | 11 | 508 | 55.3 | 6.61 |
| | mitochondrial OS = Glycine max | | | | | | | | |
| | GN = ATPA PE = 3 SV = 1 - | | | | | | | | |
| | [ATPAM_SOYBN] | | | | | | | | |
| C0J3I3 | ATP synthase subunit beta | 12.77 | 6 | 4 | 5 | 5 | 470 | 50.6 | 5.36 |
| | (Fragment) OS = Glycine max | | | | | | | | |
| | GN = atpB PE = 3 SV = 1 - | | | | | | | | |
| | [C0J3I3_SOYBN] | | | | | | | | |
| I1NFS4 | ATP synthase subunit beta | 35.96 | 4 | 13 | 14 | 22 | 559 | 59.8 | 6.15 |
| | OS = Glycine max PE = 3 SV = 1 - | | | | | | | | |
| | [I1NFS4_SOYBN] | | | | | | | | |
| K7KU09 | ATP-dependent | 7.10 | 16 | 3 | 3 | 3 | 507 | 55.9 | 6.76 |
| | 6-phosphofructokinase | | | | | | | | |
| | OS = Glycine max GN = PFK | | | | | | | | |
| | PE = 3 SV = 1 - | | | | | | | | |
| | [K7KU09_SOYBN] | | | | | | | | |
| I1KNN0 | ATP-dependent | 4.58 | 1 | 2 | 2 | 2 | 459 | 51.1 | 6.71 |
| | 6-phosphofructokinase | | | | | | | | |
| | OS = Glycine max GN = PFK | | | | | | | | |
| | PE = 3 SV = 2 - | | | | | | | | |
| | [I1KNN0_SOYBN] | | | | | | | | |
| Q8RVH5 | Basic 7S globulin 2 | 47.81 | 1 | 5 | 10 | 41 | 433 | 47.2 | 8.25 |
| | OS = Glycine max PE = 1 SV = 1 - | | | | | | | | |
| | [7SBG2_SOYBN] | | | | | | | | |
| P13917 | Basic 7S globulin OS = Glycine | 45.90 | 1 | 7 | 12 | 66 | 427 | 46.4 | 8.32 |
| | max GN = BG PE = 1 SV = 2 - | | | | | | | | |
| | [7SB1_SOYBN] | | | | | | | | |
| O49013 | Beta-1,3-glucanase 5 | 11.34 | 5 | 1 | 1 | 1 | 238 | 25.5 | 8.31 |
| | (Fragment) OS = Glycine max | | | | | | | | |
| | GN = SGlu5 PE = 3 SV = 1 - | | | | | | | | |
| | [O49013_SOYBN] | | | | | | | | |
| Q42795 | Beta-amylase OS = Glycine max | 53.43 | 13 | 21 | 21 | 71 | 496 | 56.0 | 5.55 |
| | PE = 1 SV = 1 - | | | | | | | | |
| | [Q42795_SOYBN] | | | | | | | | |
| Q948X9 | Beta-conglycinin alpha-subunit | 39.00 | 3 | 1 | 25 | 188 | 623 | 72.4 | 5.40 |
| | OS = Glycine max PE = 2 SV = 1 - | | | | | | | | |
| | [Q948X9_SOYBN] | | | | | | | | |
| Q50JD8 | Beta-conglycinin beta subunit | 53.57 | 9 | 1 | 24 | 155 | 420 | 48.3 | 5.92 |
| | (Fragment) OS = Glycine max | | | | | | | | |
| | PE = 2 SV = 1 - | | | | | | | | |
| | [Q50JD8_SOYBN] | | | | | | | | |
| F7J077 | Beta-conglycinin beta subunit | 51.25 | 2 | 1 | 24 | 157 | 439 | 50.4 | 6.24 |
| | OS = Glycine max | | | | | | | | |
| | GN = CG-beta-2 PE = 4 SV = 1 - | | | | | | | | |
| | [F7J077_SOYBN] | | | | | | | | |
| P11827 | Beta-conglycinin, alpha′ chain | 41.78 | 1 | 3 | 27 | 160 | 639 | 74.3 | 5.66 |
| | OS = Glycine max GN = CG-1 | | | | | | | | |
| | PE = 2 SV = 1 - | | | | | | | | |
| | [GLCAP_SOYBN] | | | | | | | | |
| I1LKE7 | Beta-galactosidase OS = Glycine | 15.91 | 8 | 7 | 9 | 11 | 836 | 91.1 | 7.28 |
| | max PE = 3 SV = 1 - | | | | | | | | |
| | [I1LKE7_SOYBN] | | | | | | | | |
| I1M4L7 | Beta-galactosidase OS = Glycine | 7.98 | 10 | 2 | 4 | 5 | 840 | 91.6 | 6.76 |
| | max PE = 3 SV = 1 - | | | | | | | | |
| | [I1M4L7_SOYBN] | | | | | | | | |
| Q6X0N7 | Beta-galactosidase OS = Glycine | 2.64 | 1 | 1 | 1 | 1 | 909 | 102.4 | 6.57 |
| | max PE = 3 SV = 1 - | | | | | | | | |
| | [Q6X0N7_SOYBN] | | | | | | | | |
| I1M878 | Beta-galactosidase OS = Glycine | 2.27 | 1 | 1 | 1 | 1 | 440 | 48.5 | 8.51 |
| | max PE = 4 SV = 1 - | | | | | | | | |
| | [I1M878_SOYBN] | | | | | | | | |
| I1JDS6 | Beta-hexosaminidase | 5.95 | 2 | 1 | 2 | 2 | 555 | 62.9 | 5.95 |
| | OS = Glycine max PE = 3 SV = 1 - | | | | | | | | |
| | [I1JDS6_SOYBN] | | | | | | | | |
| I1LFD4 | Beta-hexosaminidase | 6.08 | 2 | 1 | 2 | 2 | 543 | 61.3 | 8.02 |
| | OS = Glycine max PE = 3 SV = 2 - | | | | | | | | |
| | [I1LFD4_SOYBN] | | | | | | | | |
| Q9M508 | Beta-ketoacyl-ACP synthetase I | 7.46 | 2 | 2 | 2 | 2 | 469 | 49.7 | 7.49 |
| | OS = Glycine max PE = 2 SV = 1 - | | | | | | | | |
| | [Q9M508_SOYBN] | | | | | | | | |
| Q9SBA9 | Bowman-Birk proteinase | 76.19 | 6 | 3 | 3 | 4 | 42 | 4.5 | 6.89 |
| | inhibitor (Fragment) | | | | | | | | |
| | OS = Glycine max PE = 2 SV = 1 - | | | | | | | | |
| | [Q9SBA9_SOYBN] | | | | | | | | |
| P01063 | Bowman-Birk type proteinase | 15.66 | 5 | 1 | 1 | 3 | 83 | 9.2 | 4.84 |
| | inhibitor C-II OS = Glycine max | | | | | | | | |
| | PE = 1 SV = 2 - [IBBC2_SOYBN] | | | | | | | | |
| O23959 | Ca+2-binding EF hand protein | 42.26 | 3 | 7 | 8 | 10 | 239 | 27.0 | 6.44 |
| | OS = Glycine max | | | | | | | | |
| | GN = GmPM13 PE = 2 SV = 1 - | | | | | | | | |
| | [O23959_SOYBN] | | | | | | | | |
| P62163 | Calmodulin-2 OS = Glycine max | 41.61 | 5 | 1 | 6 | 7 | 149 | 16.8 | 4.27 |
| | GN = CAM-2 PE = 1 SV = 2 - | | | | | | | | |
| | [CALM2_SOYBN] | | | | | | | | |
| Q39817 | Calnexin homolog OS = Glycine | 30.22 | 2 | 4 | 14 | 16 | 546 | 62.0 | 4.89 |
| | max PE = 2 SV = 1 - | | | | | | | | |
| | [CALX_SOYBN] | | | | | | | | |
| A0A762 | Calreticulin-1 OS = Glycine max | 45.48 | 3 | 4 | 15 | 19 | 420 | 48.1 | 4.59 |
| | GN = Gm crt-1 PE = 2 SV = 1 - | | | | | | | | |
| | [A0A762_SOYBN] | | | | | | | | |
| I1JN34 | Carboxypeptidase OS = Glycine | 4.15 | 4 | 1 | 1 | 1 | 434 | 48.6 | 5.38 |
| | max PE = 3 SV = 2 - | | | | | | | | |
| | [I1JN34_SOYBN] | | | | | | | | |
| I1K7D4 | Catalase OS = Glycine max | 9.72 | 10 | 3 | 3 | 3 | 391 | 44.7 | 6.57 |
| | PE = 3 SV = 1 - | | | | | | | | |
| | [I1K7D4_SOYBN] | | | | | | | | |
| B2CM87 | Chalcone-flavonone isomerase | 6.05 | 4 | 1 | 1 | 1 | 215 | 23.5 | 6.19 |
| | family protein (Fragment) | | | | | | | | |
| | OS = Glycine max PE = 2 SV = 1 - | | | | | | | | |
| | [B2CM87_SOYBN] | | | | | | | | |
| A6XKY2 | Chalcone-flavonone isomerase | 10.09 | 5 | 2 | 2 | 2 | 218 | 23.2 | 6.80 |
| | family protein OS = Glycine max | | | | | | | | |
| | PE = 2 SV = 1 - | | | | | | | | |
| | [A6XKY2_SOYBN] | | | | | | | | |
| H2D5S2 | Citrate synthase OS = Glycine | 2.33 | 3 | 1 | 1 | 1 | 472 | 52.3 | 8.47 |
| | max PE = 2 SV = 1 - | | | | | | | | |
| | [H2D5S2_SOYBN] | | | | | | | | |
| I1M7S3 | Clathrin heavy chain | 6.88 | 11 | 9 | 9 | 9 | 1700 | 192.4 | 5.44 |
| | OS = Glycine max PE = 3 SV = 1 - | | | | | | | | |
| | [I1M7S3_SOYBN] | | | | | | | | |
| K7KMA4 | Coatomer subunit alpha | 1.24 | 5 | 2 | 2 | 3 | 1206 | 135.2 | 7.18 |
| | OS = Glycine max PE = 4 SV = 1 - | | | | | | | | |
| | [K7KMA4_SOYBN] | | | | | | | | |
| I1KG56 | Coatomer subunit beta | 5.05 | 3 | 4 | 4 | 4 | 950 | 106.0 | 5.87 |
| | OS = Glycine max PE = 4 SV = 1 - | | | | | | | | |
| | [I1KG56_SOYBN] | | | | | | | | |
| I1LJE3 | Coatomer subunit gamma | 2.11 | 3 | 1 | 1 | 1 | 759 | 84.9 | 5.80 |
| | OS = Glycine max PE = 3 SV = 1 - | | | | | | | | |
| | [I1LJE3_SOYBN] | | | | | | | | |
| C6TMX6 | Cysteine synthase OS = Glycine | 7.08 | 3 | 2 | 2 | 2 | 325 | 34.4 | 5.63 |
| | max PE = 2 SV = 1 - | | | | | | | | |
| | [C6TMX6_SOYBN] | | | | | | | | |
| Q8W1A0 | Cysteine synthase OS = Glycine | 7.08 | 6 | 2 | 2 | 2 | 325 | 34.2 | 5.82 |
| | max PE = 2 SV = 1 - | | | | | | | | |
| | [Q8W1A0_SOYBN] | | | | | | | | |
| Q2PMR7 | Cytochrome b559 subunit alpha | 10.84 | 3 | 1 | 1 | 1 | 83 | 9.4 | 4.94 |
| | OS = Glycine max GN = psbE | | | | | | | | |
| | PE = 3 SV = 1 - [PSBE_SOYBN] | | | | | | | | |
| C6TMB1 | Cytochrome c oxidase subunit 2 | 5.32 | 3 | 1 | 1 | 2 | 376 | 42.3 | 6.84 |
| | OS = Glycine max PE = 2 SV = 1 - | | | | | | | | |
| | [C6TMB1_SOYBN] | | | | | | | | |
| H9TVI9 | Cytochrome P450 CYP74C | 3.78 | 5 | 2 | 2 | 2 | 476 | 53.4 | 6.87 |
| | (Fragment) OS = Glycine max | | | | | | | | |
| | PE = 4 SV = 1 - | | | | | | | | |
| | [H9TVI9_SOYBN] | | | | | | | | |
| Q2LAJ5 | Cytochrome P450 | 2.26 | 2 | 1 | 1 | 1 | 487 | 54.8 | 8.12 |
| | monooxygenase CYP74A1 | | | | | | | | |
| | OS = Glycine max | | | | | | | | |
| | GN = CYP74A1 PE = 2 SV = 1 - | | | | | | | | |
| | [Q2LAJ5_SOYBN] | | | | | | | | |
| Q07502 | Defensin-like protein | 32.43 | 3 | 2 | 2 | 2 | 74 | 8.4 | 8.62 |
| | OS = Glycine max PE = 3 SV = 1 - | | | | | | | | |
| | [DEF_SOYBN] | | | | | | | | |
| A1KR24 | Dehydrin OS = Glycine max | 40.50 | 5 | 7 | 9 | 12 | 242 | 25.4 | 6.61 |
| | GN = LEA-2-D11 PE = 4 SV = 1 - | | | | | | | | |
| | [A1KR24_SOYBN] | | | | | | | | |
| Q39805 | Dehydrin-like protein | 85.84 | 8 | 10 | 12 | 23 | 226 | 23.7 | 6.58 |
| | OS = Glycine max PE = 2 SV = 1 - | | | | | | | | |
| | [Q39805_SOYBN] | | | | | | | | |
| I1LFD6 | DHAR class glutathione | 56.81 | 3 | 3 | 9 | 11 | 213 | 23.4 | 6.43 |
| | S-transferase OS = Glycine max | | | | | | | | |
| | GN = DHAR1 PE = 2 SV = 1 - | | | | | | | | |
| | [I1LFD6_SOYBN] | | | | | | | | |
| D2DKF0 | Dihydroxyacid dehydratase | 3.49 | 2 | 2 | 2 | 3 | 601 | 64.0 | 6.23 |
| | OS = Glycine max PE = 2 SV = 1 - | | | | | | | | |
| | [D2DKF0_SOYBN] | | | | | | | | |
| C6ZS00 | Disease resistance protein/LRR | 14.11 | 3 | 6 | 6 | 6 | 489 | 53.8 | 8.44 |
| | protein-related protein | | | | | | | | |
| | OS = Glycine max PE = 2 SV = 1 - | | | | | | | | |
| | [C6ZS00_SOYBN] | | | | | | | | |
| I1LD04 | Dolichyl-diphosphooligosaccharide-- | 4.57 | 3 | 2 | 2 | 2 | 438 | 49.1 | 8.09 |
| | protein glycosyltransferase | | | | | | | | |
| | subunit 1 OS = Glycine max | | | | | | | | |
| | PE = 3 SV = 1 - | | | | | | | | |
| | [I1LD04_SOYBN] | | | | | | | | |
| I1JSS2 | Dolichyl-diphosphooligosaccharide-- | 5.49 | 3 | 3 | 3 | 3 | 619 | 69.0 | 8.34 |
| | protein glycosyltransferase | | | | | | | | |
| | subunit 1 OS = Glycine max | | | | | | | | |
| | PE = 3 SV = 2 - [I1JSS2_SOYBN] | | | | | | | | |
| C6TNT2 | EF1Bgamma class glutathione | 34.61 | 4 | 4 | 12 | 17 | 419 | 47.6 | 6.24 |
| | S-transferase OS = Glycine max | | | | | | | | |
| | GN = EF1Bgamma3 PE = 2 SV = 1 - | | | | | | | | |
| | [C6TNT2_SOYBN] | | | | | | | | |
| C6EVF9 | Elongation factor 1-alpha | 32.44 | 4 | 9 | 11 | 23 | 447 | 49.4 | 8.92 |
| | OS = Glycine max GN = EF-1A | | | | | | | | |
| | PE = 2 SV = 1 - | | | | | | | | |
| | [C6EVF9_SOYBN] | | | | | | | | |
| I1JXC1 | Elongation factor Tu | 11.04 | 6 | 1 | 4 | 4 | 480 | 52.2 | 6.68 |
| | OS = Glycine max PE = 3 SV = 1 - | | | | | | | | |
| | [I1JXC1_SOYBN] | | | | | | | | |
| I1KCA8 | Elongation factor Tu | 11.69 | 6 | 1 | 4 | 4 | 479 | 52.1 | 6.68 |
| | OS = Glycine max PE = 3 SV = 1 - | | | | | | | | |
| | [I1KCA8_SOYBN] | | | | | | | | |
| I1N9E6 | Elongation factor Tu | 6.86 | 5 | 1 | 2 | 2 | 452 | 49.1 | 6.87 |
| | OS = Glycine max PE = 3 SV = 1 - | | | | | | | | |
| | [I1N9E6_SOYBN] | | | | | | | | |
| I1M167 | Elongation factor Tu | 7.56 | 2 | 1 | 2 | 2 | 450 | 49.1 | 7.33 |
| | OS = Glycine max PE = 3 SV = 2 - | | | | | | | | |
| | [I1M167_SOYBN] | | | | | | | | |
| O49857 | Epoxide hydrolase OS = Glycine | 17.30 | 13 | 5 | 5 | 5 | 341 | 39.1 | 5.66 |
| | max PE = 4 SV = 1 - | | | | | | | | |
| | [O49857_SOYBN] | | | | | | | | |
| I1JPD4 | Eukaryotic translation initiation | 10.54 | 3 | 6 | 6 | 6 | 958 | 111.9 | 9.29 |
| | factor 3 subunit A OS = Glycine | | | | | | | | |
| | max PE = 3 SV = 1 - | | | | | | | | |
| | [I1JPD4_SOYBN] | | | | | | | | |
| I1JK05 | Eukaryotic translation initiation | 8.91 | 3 | 6 | 6 | 6 | 718 | 82.7 | 5.22 |
| | factor 3 subunit B OS = Glycine | | | | | | | | |
| | max PE = 3 SV = 1 - | | | | | | | | |
| | [I1JK05_SOYBN] | | | | | | | | |
| I1JQD9 | Eukaryotic translation initiation | 4.43 | 3 | 4 | 4 | 4 | 926 | 104.6 | 5.63 |
| | factor 3 subunit C OS = Glycine | | | | | | | | |
| | max PE = 3 SV = 1 - | | | | | | | | |
| | [I1JQD9_SOYBN] | | | | | | | | |
| I1MEK0 | Eukaryotic translation initiation | 5.66 | 3 | 2 | 2 | 2 | 565 | 64.3 | 5.71 |
| | factor 3 subunit D OS = Glycine | | | | | | | | |
| | max PE = 3 SV = 1 - | | | | | | | | |
| | [I1MEK0_SOYBN] | | | | | | | | |
| C6TC72 | Eukaryotic translation initiation | 13.15 | 2 | 2 | 3 | 3 | 289 | 31.7 | 5.34 |
| | factor 3 subunit F OS = Glycine | | | | | | | | |
| | max PE = 2 SV = 1 - | | | | | | | | |
| | [C6TC72_SOYBN] | | | | | | | | |
| I1LMP5 | Eukaryotic translation initiation | 10.36 | 4 | 1 | 2 | 2 | 280 | 30.7 | 5.33 |
| | factor 3 subunit F OS = Glycine | | | | | | | | |
| | max PE = 3 SV = 1 - | | | | | | | | |
| | [I1LMP5_SOYBN] | | | | | | | | |
| I1KXJ9 | Eukaryotic translation initiation | 6.21 | 2 | 2 | 2 | 2 | 290 | 32.1 | 8.21 |
| | factor 3 subunit G OS = Glycine | | | | | | | | |
| | max PE = 3 SV = 1 - | | | | | | | | |
| | [I1KXJ9_SOYBN] | | | | | | | | |
| C6TL41 | Eukaryotic translation initiation | 10.74 | 3 | 2 | 2 | 2 | 326 | 35.9 | 6.76 |
| | factor 3 subunit I OS = Glycine | | | | | | | | |
| | max PE = 2 SV = 1 - | | | | | | | | |
| | [C6TL41_SOYBN] | | | | | | | | |
| I1JUS7 | Eukaryotic translation initiation | 1.36 | 2 | 1 | 1 | 1 | 514 | 60.5 | 6.19 |
| | factor 3 subunit L OS = Glycine | | | | | | | | |
| | max PE = 3 SV = 1 - | | | | | | | | |
| | [I1JUS7_SOYBN] | | | | | | | | |
| C6SVE0 | Eukaryotic translation initiation | 30.00 | 10 | 1 | 4 | 4 | 160 | 17.4 | 5.76 |
| | factor 5A OS = Glycine max | | | | | | | | |
| | PE = 2 SV = 1 - | | | | | | | | |
| | [C6SVE0_SOYBN] | | | | | | | | |
| C6ZHS4 | Eukaryotic translation initiation | 44.38 | 12 | 2 | 5 | 6 | 160 | 17.5 | 5.99 |
| | factor 5A OS = Glycine max | | | | | | | | |
| | PE = 2 SV = 1 - | | | | | | | | |
| | [C6ZHS4_SOYBN] | | | | | | | | |
| C6TCN5 | Ferritin OS = Glycine max PE = 2 | 24.00 | 13 | 4 | 6 | 8 | 250 | 28.1 | 6.04 |
| | SV = 1 - [C6TCN5_SOYBN] | | | | | | | | |
| I1J7H3 | Ferritin OS = Glycine max PE = 3 | 26.07 | 6 | 4 | 5 | 7 | 257 | 28.7 | 6.18 |
| | SV = 1 - [I1J7H3_SOYBN] | | | | | | | | |
| Q948P5 | Ferritin-4, chloroplastic | 21.46 | 11 | 3 | 4 | 5 | 247 | 27.5 | 5.96 |
| | OS = Glycine max PE = 1 SV = 2 - | | | | | | | | |
| | [FRI4_SOYBN] | | | | | | | | |
| I1JH86 | Fructose-bisphosphate aldolase | 45.25 | 8 | 4 | 13 | 22 | 358 | 38.2 | 7.55 |
| | OS = Glycine max PE = 3 SV = 1 - | | | | | | | | |
| | [I1JH86_SOYBN] | | | | | | | | |
| I1JPX8 | Fructose-bisphosphate aldolase | 20.11 | 5 | 2 | 6 | 8 | 358 | 38.5 | 6.79 |
| | OS = Glycine max PE = 3 SV = 1 - | | | | | | | | |
| | [I1JPX8_SOYBN] | | | | | | | | |
| I1LPX6 | Fructose-bisphosphate aldolase | 18.59 | 11 | 6 | 6 | 6 | 398 | 42.8 | 7.33 |
| | OS = Glycine max PE = 3 SV = 1 - | | | | | | | | |
| | [I1LPX6_SOYBN] | | | | | | | | |
| I1MB71 | Fructose-bisphosphate aldolase | 45.81 | 9 | 3 | 13 | 22 | 358 | 38.3 | 7.53 |
| | OS = Glycine max PE = 3 SV = 1 - | | | | | | | | |
| | [I1MB71_SOYBN] | | | | | | | | |
| C7S917 | Germin-like protein 21 | 7.03 | 4 | 1 | 1 | 1 | 185 | 195. | 9.60 |
| | OS = Glycine max GN = GLP21 | | | | | | | | |
| | PE = 2 SV = 1 - | | | | | | | | |
| | [C7S917_SOYBN] | | | | | | | | |
| F8SMC3 | Glucose-6-phosphate | 2.32 | 1 | 1 | 1 | 1 | 518 | 59.3 | 6.77 |
| | 1-dehydrogenase OS = Glycine | | | | | | | | |
| | max PE = 2 SV = 1 - | | | | | | | | |
| | [F8SMC3_SOYBN] | | | | | | | | |
| C6TGC6 | Glucose-6-phosphate isomerase | 6.69 | 5 | 3 | 3 | 3 | 568 | 62.6 | 6.33 |
| | OS = Glycine max PE = 2 SV = 1 - | | | | | | | | |
| | [C6TGC6_SOYBN] | | | | | | | | |
| K7KU59 | Glucose-6-phosphate isomerase | 3.21 | 5 | 2 | 2 | 2 | 592 | 64.9 | 5.63 |
| | OS = Glycine max PE = 3 SV = 1 - | | | | | | | | |
| | [K7KU59_SOYBN] | | | | | | | | |
| C6TDZ0 | Glutamate dehydrogenase | 5.35 | 8 | 2 | 2 | 2 | 411 | 44.5 | 6.40 |
| | OS = Glycine max PE = 2 SV = 1 - | | | | | | | | |
| | [C6TDZ0_SOYBN] | | | | | | | | |
| P24099 | Glutamine synthetase cytosolic | 9.86 | 17 | 3 | 3 | 3 | 355 | 38.8 | 5.71 |
| | isozyme 1 OS = Glycine max | | | | | | | | |
| | PE = 2 SV = 1 - | | | | | | | | |
| | [GLNA1_SOYBN] | | | | | | | | |
| I1K6J6 | Glutathione peroxidase | 17.47 | 7 | 1 | 3 | 6 | 166 | 18.4 | 7.08 |
| | OS = Glycine max PE = 3 SV = 1 - | | | | | | | | |
| | [I1K6J6_SOYBN] | | | | | | | | |
| I1LGG2 | Glutathione peroxidase | 28.14 | 9 | 3 | 5 | 8 | 167 | 18.5 | 6.19 |
| | OS = Glycine max PE = 3 SV = 1 - | | | | | | | | |
| | [I1LGG2_SOYBN] | | | | | | | | |
| P48640 | Glutathione reductase, | 2.94 | 4 | 1 | 1 | 1 | 544 | 58.7 | 8.25 |
| | chloroplastic OS = Glycine max | | | | | | | | |
| | GN = GR PE = 2 SV = 1 - | | | | | | | | |
| | [GSHRP_SOYBN] | | | | | | | | |
| Q9FQE4 | Glutathione S-transferase GST | 12.33 | 10 | 2 | 3 | 3 | 219 | 25.3 | 7.59 |
| | 14 (Fragment) OS = Glycine max | | | | | | | | |
| | PE = 2 SV = 1 - | | | | | | | | |
| | [Q9FQE4_SOYBN] | | | | | | | | |
| B2YDR0 | Gly m Bd 28K allergen | 31.21 | 6 | 11 | 11 | 36 | 455 | 50.7 | 6.19 |
| | (Fragment) OS = Glycine max | | | | | | | | |
| | PE = 4 SV = 1 - | | | | | | | | |
| | [B2YDR0_SOYBN] | | | | | | | | |
| Q2I0H4 | Glyceraldehyde-3-phosphate | 55.33 | 10 | 1 | 15 | 51 | 338 | 36.7 | 7.24 |
| | dehydrogenase OS = Glycine | | | | | | | | |
| | max GN = GAPC1 PE = 2 SV = 1 - | | | | | | | | |
| | [Q2I0H4_SOYBN] | | | | | | | | |
| C6T9R8 | Glyceraldehyde-3-phosphate | 7.10 | 7 | 1 | 3 | 3 | 451 | 48.1 | 7.43 |
| | dehydrogenase OS = Glycine | | | | | | | | |
| | max PE = 2 SV = 1 - | | | | | | | | |
| | [C6T9R8_SOYBN] | | | | | | | | |
| C6TD56 | Glyceraldehyde-3-phosphate | 50.59 | 2 | 2 | 14 | 40 | 340 | 37.0 | 7.47 |
| | dehydrogenase OS = Glycine | | | | | | | | |
| | max PE = 2 SV = 1 - | | | | | | | | |
| | [C6TD56_SOYBN] | | | | | | | | |
| Q07CZ3 | Glyceraldehyde-3-phosphate | 53.25 | 5 | 1 | 15 | 45 | 338 | 36.7 | 7.24 |
| | dehydrogenase OS = Glycine | | | | | | | | |
| | max PE = 2 SV = 1 - | | | | | | | | |
| | [Q07CZ3_SOYBN] | | | | | | | | |
| I1ML66 | Glyceraldehyde-3-phosphate | 15.14 | 6 | 4 | 6 | 6 | 403 | 43.2 | 7.75 |
| | dehydrogenase OS = Glycine | | | | | | | | |
| | max PE = 3 SV = 1 - | | | | | | | | |
| | [I1ML66_SOYBN] | | | | | | | | |
| I1MYI8 | Glyceraldehyde-3-phosphate | 50.59 | 5 | 2 | 14 | 42 | 338 | 36.8 | 7.47 |
| | dehydrogenase OS = Glycine | | | | | | | | |
| | max PE = 3 SV = 1 - | | | | | | | | |
| | [I1MYI8_SOYBN] | | | | | | | | |
| K7KL44 | Glyceraldehyde-3-phosphate | 43.90 | 6 | 4 | 16 | 51 | 426 | 46.7 | 7.72 |
| | dehydrogenase OS = Glycine | | | | | | | | |
| | max PE = 3 SV = 1 - | | | | | | | | |
| | [K7KL44_SOYBN] | | | | | | | | |
| P04776 | Glycinin G1 OS = Glycine max | 60.40 | 5 | 14 | 21 | 245 | 495 | 55.7 | 6.23 |
| | GN = GY1 PE = 1 SV = 2 - | | | | | | | | |
| | [GLYG1_SOYBN] | | | | | | | | |
| P04405 | Glycinin G2 OS = Glycine max | 67.42 | 2 | 15 | 21 | 357 | 485 | 54.4 | 5.58 |
| | GN = Gy2 PE = 1 SV = 2 - | | | | | | | | |
| | [GLYG2_SOYBN] | | | | | | | | |
| P11828 | Glycinin G3 OS = Glycine max | 77.13 | 3 | 11 | 20 | 200 | 481 | 54.2 | 5.97 |
| | GN = GY3 PE = 3 SV = 1 - | | | | | | | | |
| | [GLYG3_SOYBN] | | | | | | | | |
| Q9SB11 | Glycinin OS = Glycine max | 57.37 | 5 | 16 | 22 | 178 | 563 | 63.8 | 5.29 |
| | GN = A5A4B3 PE = 2 SV = 1 - | | | | | | | | |
| | [Q9SB11_SOYBN] | | | | | | | | |
| P93708 | Glycinin OS = Glycine max | 65.76 | 2 | 2 | 20 | 326 | 517 | 58.2 | 5.69 |
| | GN = Gly A3B4 PE = 2 SV = 1 - | | | | | | | | |
| | [P93708_SOYBN] | | | | | | | | |
| P04347 | Glycinin OS = Glycine max | 52.71 | 1 | 2 | 18 | 259 | 516 | 57.9 | 5.90 |
| | PE = 1 SV = 1 - | | | | | | | | |
| | [GLYG5_SOYBN] | | | | | | | | |
| Q9SB12 | Glycinin OS = Glycine max | 70.60 | 3 | 3 | 22 | 324 | 517 | 58.2 | 5.78 |
| | PE = 4 SV = 1 - | | | | | | | | |
| | [Q9SB12_SOYBN] | | | | | | | | |
| I1KN68 | Glycosyltransferase | 4.62 | 3 | 2 | 2 | 2 | 476 | 53.2 | 6.90 |
| | OS = Glycine max GN = GmSg-1 | | | | | | | | |
| | PE = 3 SV = 1 - | | | | | | | | |
| | [I1KN68_SOYBN] | | | | | | | | |
| C6T7G2 | Glycosyltransferase | 3.13 | 2 | 1 | 1 | 1 | 447 | 49.0 | 6.42 |
| | OS = Glycine max PE = 2 SV = 1 - | | | | | | | | |
| | [C6T7G2_SOYBN] | | | | | | | | |
| I1JE17 | Glycosyltransferase | 4.63 | 1 | 2 | 2 | 2 | 475 | 53.1 | 5.86 |
| | OS = Glycine max PE = 3 SV = 1 - | | | | | | | | |
| | [I1JE17_SOYBN] | | | | | | | | |
| I1L932 | Glycosyltransferase | 2.26 | 1 | 1 | 1 | 1 | 486 | 54.4 | 5.40 |
| | OS = Glycine max PE = 3 SV = 1 - | | | | | | | | |
| | [I1L932_SOYBN] | | | | | | | | |
| I1KYQ0 | Glycosyltransferase | 2.00 | 7 | 1 | 1 | 1 | 450 | 49.2 | 6.00 |
| | OS = Glycine max PE = 3 SV = 2 - | | | | | | | | |
| | [I1KYQ0_SOYBN] | | | | | | | | |
| I1LEK0 | Glycosyltransferase | 2.09 | 1 | 1 | 1 | 1 | 527 | 58.5 | 6.21 |
| | OS = Glycine max PE = 3 SV = 2 - | | | | | | | | |
| | [I1LEK0_SOYBN] | | | | | | | | |
| B4XH50 | Glycylpeptide | 15.63 | 4 | 1 | 1 | 1 | 96 | 10.8 | 8.91 |
| | N-tetradecanoyltransferase | | | | | | | | |
| | (Fragment) OS = Glycine max | | | | | | | | |
| | PE = 3 SV = 1 - | | | | | | | | |
| | [B4XH50_SOYBN] | | | | | | | | |
| B6EBD6 | Heat shock protein 90-2 | 24.86 | 13 | 4 | 15 | 16 | 700 | 80.1 | 5.07 |
| | OS = Glycine max PE = 2 SV = 1 - | | | | | | | | |
| | [B6EBD6_SOYBN] | | | | | | | | |
| I1K5Z4 | Hexokinase OS = Glycine max | 2.21 | 2 | 1 | 1 | 1 | 498 | 53.7 | 5.85 |
| | PE = 3 SV = 1 - | | | | | | | | |
| | [I1K5Z4_SOYBN] | | | | | | | | |
| I1MG54 | Histidinol dehydrogenase, | 1.49 | 1 | 1 | 1 | 1 | 471 | 51.3 | 6.76 |
| | chloroplastic OS = Glycine max | | | | | | | | |
| | PE = 3 SV = 1 - | | | | | | | | |
| | [I1MG54_SOYBN] | | | | | | | | |
| C6T2B0 | Histone H2A OS = Glycine max | 20.31 | 16 | 2 | 3 | 3 | 128 | 13.7 | 10.36 |
| | PE = 2 SV = 1 - | | | | | | | | |
| | [C6T2B0_SOYBN] | | | | | | | | |
| C6TMV8 | Histone H2A OS = Glycine max | 21.62 | 14 | 1 | 2 | 2 | 148 | 15.6 | 10.67 |
| | PE = 2 SV = 1 - | | | | | | | | |
| | [C6TMV8_SOYBN] | | | | | | | | |
| C6SWE9 | Histone H2B OS = Glycine max | 25.55 | 7 | 3 | 3 | 4 | 137 | 15.1 | 10.05 |
| | PE = 2 SV = 1 - | | | | | | | | |
| | [C6SWE9_SOYBN] | | | | | | | | |
| Q42782 | Histone H3 (Fragment) | 22.83 | 14 | 1 | 4 | 4 | 127 | 14.3 | 10.73 |
| | OS = Glycine max PE = 3 SV = 1 - | | | | | | | | |
| | [Q42782_SOYBN] | | | | | | | | |
| C6SZA4 | Histone H3 OS = Glycine max | 21.32 | 6 | 1 | 4 | 4 | 136 | 15.3 | 11.30 |
| | PE = 2 SV = 1 - | | | | | | | | |
| | [C6SZA4_SOYBN] | | | | | | | | |
| P0CG89 | Histone H4 OS = Glycine max | 38.83 | 8 | 4 | 4 | 5 | 103 | 11.4 | 11.47 |
| | PE = 3 SV = 1 - [H4_SOYBN] | | | | | | | | |
| D6C4Z9 | HSP90-1 OS = Glycine max | 22.36 | 5 | 1 | 13 | 14 | 702 | 80.3 | 5.02 |
| | PE = 2 SV = 1 - | | | | | | | | |
| | [D6C4Z9_SOYBN] | | | | | | | | |
| D6C500 | HSP90-2 OS = Glycine max | 24.61 | 7 | 1 | 14 | 15 | 699 | 80.1 | 5.06 |
| | PE = 2 SV = 1 - | | | | | | | | |
| | [D6C500_SOYBN] | | | | | | | | |
| P24337 | Hydrophobic seed protein | 26.25 | 4 | 2 | 2 | 5 | 80 | 8.4 | 6.28 |
| | OS = Glycine max PE = 1 SV = 1 - | | | | | | | | |
| | [HPSE_SOYBN] | | | | | | | | |
| I1MJD7 | Inosine-5′-monophosphate | 7.17 | 5 | 3 | 3 | 3 | 502 | 53.3 | 6.11 |
| | dehydrogenase OS = Glycine | | | | | | | | |
| | max PE = 3 SV = 1 - | | | | | | | | |
| | [I1MJD7_SOYBN] | | | | | | | | |
| A7X657 | Inositol-tetrakisphosphate | 5.01 | 1 | 1 | 1 | 1 | 339 | 38.4 | 6.09 |
| | 1-kinase OS = Glycine max | | | | | | | | |
| | GN = Itpk1 PE = 2 SV = 1 - | | | | | | | | |
| | [A7X657_SOYBN] | | | | | | | | |
| I1JHS8 | Isocitrate dehydrogenase | 7.12 | 10 | 3 | 3 | 3 | 393 | 43.8 | 6.55 |
| | [NADP] OS = Glycine max | | | | | | | | |
| | PE = 3 SV = 1 - | | | | | | | | |
| | [I1JHS8_SOYBN] | | | | | | | | |
| Q9SDZ0 | Isoflavone reductase homolog 2 | 37.74 | 17 | 10 | 10 | 13 | 310 | 33.9 | 5.88 |
| | OS = Glycine max GN = IFR2 | | | | | | | | |
| | PE = 2 SV = 1 - | | | | | | | | |
| | [Q9SDZ0_SOYBN] | | | | | | | | |
| I1LUL9 | Ketol-acid reductoisomerase | 16.04 | 3 | 4 | 7 | 9 | 586 | 63.3 | 7.30 |
| | OS = Glycine max PE = 3 SV = 1 - | | | | | | | | |
| | [I1LUL9_SOYBN] | | | | | | | | |
| I1KZT2 | Lactoylglutathione lyase | 27.53 | 8 | 2 | 8 | 9 | 287 | 32.4 | 6.01 |
| | OS = Glycine max PE = 3 SV = 1 - | | | | | | | | |
| | [I1KZT2_SOYBN] | | | | | | | | |
| I1LR74 | Lactoylglutathione lyase | 5.41 | 5 | 1 | 1 | 1 | 185 | 21.0 | 5.64 |
| | OS = Glycine max PE = 3 SV = 1 - | | | | | | | | |
| | [I1LR74_SOYBN] | | | | | | | | |
| I1MFH9 | Lactoylglutathione lyase | 23.00 | 14 | 1 | 7 | 8 | 287 | 32.4 | 6.01 |
| | OS = Glycine max PE = 3 SV = 1 - | | | | | | | | |
| | [I1MFH9_SOYBN] | | | | | | | | |
| I1L8Q0 | Lambda class glutathione | 14.77 | 5 | 3 | 3 | 3 | 237 | 27.1 | 5.52 |
| | S-transferase OS = Glycine max | | | | | | | | |
| | GN = GSTL3 PE = 2 SV = 1 - | | | | | | | | |
| | [I1L8Q0_SOYBN] | | | | | | | | |
| I1NA39 | Lambda class glutathione | 33.76 | 3 | 6 | 6 | 7 | 237 | 27.0 | 5.71 |
| | S-transferase OS = Glycine max | | | | | | | | |
| | GN = GSTL7 PE = 2 SV = 1 - | | | | | | | | |
| | [I1NA39_SOYBN] | | | | | | | | |
| Q39871 | Late embryongenesis abundant | 28.73 | 2 | 5 | 11 | 15 | 463 | 50.6 | 6.67 |
| | protein OS = Glycine max | | | | | | | | |
| | GN = MP2 PE = 2 SV = 1 - | | | | | | | | |
| | [Q39871_SOYBN] | | | | | | | | |
| Q9ZTZ2 | Late-embryogenesis abundant | 6.02 | 1 | 1 | 1 | 1 | 133 | 14.7 | 9.29 |
| | protein OS = Glycine max | | | | | | | | |
| | GN = PM16 PE = 2 SV = 1 - | | | | | | | | |
| | [Q9ZTZ2_SOYBN] | | | | | | | | |
| P05046 | Lectin OS = Glycine max | 29.82 | 1 | 6 | 6 | 15 | 285 | 30.9 | 6.05 |
| | GN = LE1 PE = 1 SV = 1 - | | | | | | | | |
| | [LEC_SOYBN] | | | | | | | | |
| Q39874 | Lipoxygenase (Fragment) | 39.65 | 1 | 1 | 21 | 67 | 623 | 70.5 | 8.73 |
| | OS = Glycine max PE = 2 SV = 1 - | | | | | | | | |
| | [Q39874_SOYBN] | | | | | | | | |
| B3TDK6 | Lipoxygenase OS = Glycine max | 55.31 | 8 | 34 | 44 | 217 | 857 | 96.7 | 6.58 |
| | GN = Lx3 PE = 3 SV = 1 - | | | | | | | | |
| | [B3TDK6_SOYBN] | | | | | | | | |
| B3TDK4 | Lipoxygenase OS = Glycine max | 56.26 | 6 | 31 | 37 | 156 | 839 | 94.4 | 6.33 |
| | PE = 3 SV = 1 - | | | | | | | | |
| | [B3TDK4_SOYBN] | | | | | | | | |
| B3TDK5 | Lipoxygenase OS = Glycine max | 54.99 | 7 | 12 | 36 | 114 | 862 | 96.7 | 6.64 |
| | PE = 3 SV = 1 - | | | | | | | | |
| | [B3TDK5_SOYBN] | | | | | | | | |
| B3TDK8 | Lipoxygenase OS = Glycine max | 10.11 | 11 | 2 | 7 | 24 | 801 | 91.2 | 6.52 |
| | PE = 3 SV = 1 - | | | | | | | | |
| | [B3TDK8_SOYBN] | | | | | | | | |
| I1KUQ3 | Lipoxygenase OS = Glycine max | 4.61 | 2 | 1 | 4 | 6 | 867 | 97.7 | 6.00 |
| | PE = 3 SV = 1 - | | | | | | | | |
| | [I1KUQ3_SOYBN] | | | | | | | | |
| I1KUQ6 | Lipoxygenase OS = Glycine max | 4.95 | 2 | 2 | 4 | 9 | 868 | 97.1 | 7.40 |
| | PE = 3 SV = 2 - | | | | | | | | |
| | [I1KUQ6_SOYBN] | | | | | | | | |
| K7KN32 | L-lactate dehydrogenase | 15.01 | 3 | 6 | 6 | 8 | 413 | 44.9 | 7.64 |
| | OS = Glycine max PE = 3 SV = 1 - | | | | | | | | |
| | [K7KN32_SOYBN] | | | | | | | | |
| Q9SPB8 | Malate dehydrogenase | 39.13 | 6 | 6 | 8 | 10 | 345 | 36.1 | 8.12 |
| | OS = Glycine max GN = Mdh1 | | | | | | | | |
| | PE = 3 SV = 1 - | | | | | | | | |
| | [Q9SPB8_SOYBN] | | | | | | | | |
| H2D5S3 | Malate dehydrogenase | 28.35 | 4 | 2 | 8 | 17 | 328 | 35.2 | 6.30 |
| | OS = Glycine max PE = 2 SV = 1 - | | | | | | | | |
| | [H2D5S3_SOYBN] | | | | | | | | |
| I1JB84 | Malate dehydrogenase | 28.99 | 4 | 1 | 7 | 16 | 307 | 32.6 | 7.40 |
| | OS = Glycine max PE = 3 SV = 1 - | | | | | | | | |
| | [I1JB84_SOYBN] | | | | | | | | |
| I1MTU1 | Malate dehydrogenase | 22.16 | 2 | 2 | 7 | 7 | 361 | 38.3 | 6.27 |
| | OS = Glycine max PE = 3 SV = 1 - | | | | | | | | |
| | [I1MTU1_SOYBN] | | | | | | | | |
| I1K089 | Malate synthase OS = Glycine | 6.12 | 4 | 3 | 3 | 3 | 556 | 62.7 | 6.29 |
| | max PE = 3 SV = 1 - | | | | | | | | |
| | [I1K089_SOYBN] | | | | | | | | |
| K7KZ23 | Malic enzyme (Fragment) | 1.96 | 7 | 1 | 1 | 1 | 459 | 51.8 | 6.48 |
| | OS = Glycine max PE = 3 SV = 1 - | | | | | | | | |
| | [K7KZ23_SOYBN] | | | | | | | | |
| A7BIC9 | Malonyl-CoA:isoflavone | 2.14 | 1 | 1 | 1 | 1 | 467 | 51.6 | 6.42 |
| | 7-O-glucoside-6″-O-malonyltransferase | | | | | | | | |
| | OS = Glycine max | | | | | | | | |
| | GN = GmIF7MaT PE = 2 SV = 1 - | | | | | | | | |
| | [A7BIC9_SOYBN] | | | | | | | | |
| I1N5B5 | Mannose-6-phosphate | 3.09 | 1 | 1 | 1 | 1 | 421 | 46.2 | 5.96 |
| | isomerase OS = Glycine max | | | | | | | | |
| | PE = 3 SV = 1 - | | | | | | | | |
| | [I1N5B5_SOYBN] | | | | | | | | |
| Q01527 | Maturation protein OS = Glycine | 20.39 | 2 | 2 | 3 | 4 | 152 | 15.6 | 9.66 |
| | max GN = gGmpm9 PE = 2 SV = 1 - | | | | | | | | |
| | [Q01527_SOYBN] | | | | | | | | |
| Q9SPJ6 | Maturation protein pPM32 | 12.14 | 2 | 2 | 2 | 3 | 173 | 18.9 | 5.60 |
| | OS = Glycine max GN = PM32 | | | | | | | | |
| | PE = 2 SV = 1 - | | | | | | | | |
| | [Q9SPJ6_SOYBN] | | | | | | | | |
| O22378 | Metallothionein-II protein | 50.59 | 2 | 3 | 3 | 4 | 85 | 8.3 | 7.33 |
| | OS = Glycine max | | | | | | | | |
| | GN = PGMPM19 PE = 2 SV = 1 - | | | | | | | | |
| | [O22378_SOYBN] | | | | | | | | |
| I1J7T5 | Methionine aminopeptidase 1 | 3.49 | 4 | 1 | 1 | 1 | 373 | 41.3 | 6.81 |
| | OS = Glycine max PE = 3 SV = 1 - | | | | | | | | |
| | [I1J7T5_SOYBN] | | | | | | | | |
| Q71EW8 | Methionine synthase | 38.40 | 6 | 15 | 22 | 35 | 763 | 84.2 | 6.33 |
| | OS = Glycine max PE = 2 SV = 1 - | | | | | | | | |
| | [Q71EW8_SOYBN] | | | | | | | | |
| I1KG06 | Methylenetetrahydrofolate | 4.02 | 5 | 1 | 1 | 1 | 473 | 53.4 | 7.34 |
| | reductase OS = Glycine max | | | | | | | | |
| | PE = 3 SV = 1 - | | | | | | | | |
| | [I1KG06_SOYBN] | | | | | | | | |
| C6TBT9 | NADH-cytochrome b5 | 7.55 | 1 | 1 | 1 | 1 | 278 | 30.9 | 8.13 |
| | reductase OS = Glycine max | | | | | | | | |
| | PE = 2 SV = 1 - | | | | | | | | |
| | [C6TBT9_SOYBN] | | | | | | | | |
| C6TKA3 | NADH-cytochrome b5 | 9.72 | 3 | 3 | 3 | 4 | 319 | 35.1 | 8.56 |
| | reductase OS = Glycine max | | | | | | | | |
| | PE = 2 SV = 1 - | | | | | | | | |
| | [C6TKA3_SOYBN] | | | | | | | | |
| C6T0Q0 | Nascent polypeptide-associated | 34.16 | 4 | 4 | 4 | 5 | 161 | 17.5 | 8.32 |
| | complex subunit beta | | | | | | | | |
| | OS = Glycine max PE = 2 SV = 1 - | | | | | | | | |
| | [C6T0Q0_SOYBN] | | | | | | | | |
| Q9XGS2 | Ni-binding urease accessory | 35.04 | 3 | 7 | 7 | 7 | 274 | 30.1 | 6.39 |
| | protein UreG OS = Glycine max | | | | | | | | |
| | GN = Eu3 PE = 2 SV = 1 - | | | | | | | | |
| | [Q9XGS2_SOYBN] | | | | | | | | |
| C6TFC1 | Non-specific lipid-transfer | 32.79 | 5 | 3 | 3 | 3 | 122 | 12.4 | 8.63 |
| | protein OS = Glycine max PE = 2 | | | | | | | | |
| | SV = 1 - [C6TFC1_SOYBN] | | | | | | | | |
| Q39839 | Nucleoside diphosphate kinase | 39.60 | 2 | 1 | 5 | 7 | 149 | 16.4 | 6.32 |
| | 1 OS = Glycine max PE = 2 SV = 1 | | | | | | | | |
| | [NDK1_SOYBN] | | | | | | | | |
| C6T3F0 | Nucleoside diphosphate kinase | 18.52 | 3 | 3 | 3 | 4 | 162 | 18.1 | 6.34 |
| | OS = Glycine max PE = 2 SV = 1 - | | | | | | | | |
| | [C6T3F0_SOYBN] | | | | | | | | |
| I1K081 | Nucleoside diphosphate kinase | 11.11 | 5 | 2 | 2 | 2 | 225 | 25.0 | 8.85 |
| | OS = Glycine max PE = 3 SV = 1 - | | | | | | | | |
| | [I1K081_SOYBN] | | | | | | | | |
| I1KJI7 | Nucleoside diphosphate kinase | 49.66 | 4 | 2 | 6 | 12 | 149 | 16.5 | 6.80 |
| | OS = Glycine max PE = 3 SV = 1 - | | | | | | | | |
| | [I1KJI7_SOYBN] | | | | | | | | |
| C6TI33 | Obg-like ATPase 1 OS = Glycine | 11.42 | 2 | 4 | 4 | 5 | 394 | 44.4 | 6.80 |
| | max PE = 2 SV = 1 - | | | | | | | | |
| | [C6TI33_SOYBN] | | | | | | | | |
| C3VHQ8 | Oleosin OS = Glycine max PE = 2 | 29.09 | 2 | 2 | 4 | 13 | 165 | 17.4 | 8.06 |
| | SV = 1 - [C3VHQ8_SOYBN] | | | | | | | | |
| I1K1K4 | Oleosin OS = Glycine max PE = 3 | 18.78 | 1 | 3 | 3 | 5 | 181 | 19.1 | 8.29 |
| | SV = 1 - [I1K1K4_SOYBN] | | | | | | | | |
| I1N5I8 | Oleosin OS = Glycine max PE = 3 | 10.58 | 1 | 2 | 2 | 2 | 189 | 19.9 | 9.20 |
| | SV = 1 - [I1N5I8_SOYBN] | | | | | | | | |
| I1N747 | Oleosin OS = Glycine max PE = 3 | 32.30 | 2 | 3 | 6 | 58 | 226 | 23.6 | 8.94 |
| | SV = 1 - [I1N747_SOYBN] | | | | | | | | |
| K7KTR9 | Oleosin OS = Glycine max PE = 3 | 18.87 | 1 | 1 | 3 | 7 | 212 | 22.6 | 8.70 |
| | SV = 1 - [K7KTR9_SOYBN] | | | | | | | | |
| P29531 | P24 oleosin isoform B | 32.29 | 1 | 3 | 6 | 54 | 223 | 23.4 | 8.94 |
| | OS = Glycine max PE = 2 SV = 1 - | | | | | | | | |
| | [OLEO2_SOYBN] | | | | | | | | |
| I1MK76 | Pectinesterase OS = Glycine max | 7.32 | 2 | 3 | 3 | 3 | 492 | 54.7 | 7.75 |
| | PE = 3 SV = 1 - | | | | | | | | |
| | [I1MK76_SOYBN] | | | | | | | | |
| Q8W171 | Peptidyl-prolyl cis-trans | 63.95 | 4 | 5 | 7 | 20 | 172 | 18.2 | 8.46 |
| | isomerase 1 OS = Glycine max | | | | | | | | |
| | GN = Cyp1 PE = 2 SV = 1 - | | | | | | | | |
| | [CYP1_SOYBN] | | | | | | | | |
| C6SV88 | Peptidyl-prolyl cis-trans | 34.69 | 4 | 2 | 4 | 4 | 147 | 15.9 | 6.32 |
| | isomerase OS = Glycine max | | | | | | | | |
| | PE = 2 SV = 1 - | | | | | | | | |
| | [C6SV88_SOYBN] | | | | | | | | |
| C6SV97 | Peptidyl-prolyl cis-trans | 3.43 | 4 | 1 | 1 | 1 | 204 | 21.9 | 9.17 |
| | isomerase OS = Glycine max | | | | | | | | |
| | PE = 2 SV = 1 - | | | | | | | | |
| | [C6SV97_SOYBN] | | | | | | | | |
| C6SYQ9 | Peptidyl-prolyl cis-trans | 4.00 | 1 | 1 | 1 | 1 | 175 | 19.0 | 8.16 |
| | isomerase OS = Glycine max | | | | | | | | |
| | PE = 2 SV = 1 - | | | | | | | | |
| | [C6SYQ9_SOYBN] | | | | | | | | |
| C6SYR2 | Peptidyl-prolyl cis-trans | 24.03 | 1 | 1 | 3 | 4 | 154 | 16.8 | 7.99 |
| | isomerase OS = Glycine max | | | | | | | | |
| | PE = 2 SV = 1 - | | | | | | | | |
| | [C6SYR2_SOYBN] | | | | | | | | |
| C6T190 | Peptidyl-prolyl cis-trans | 8.93 | 1 | 1 | 1 | 1 | 112 | 12.0 | 7.97 |
| | isomerase OS = Glycine max | | | | | | | | |
| | PE = 2 SV = 1 - | | | | | | | | |
| | [C6T190_SOYBN] | | | | | | | | |
| I1NJE9 | Peptidyl-prolyl cis-trans | 13.68 | 5 | 2 | 2 | 2 | 212 | 22.7 | 8.91 |
| | isomerase OS = Glycine max | | | | | | | | |
| | PE = 3 SV = 1 - | | | | | | | | |
| | [I1NJE9_SOYBN] | | | | | | | | |
| K7LSN8 | Peptidyl-prolyl cis-trans | 36.93 | 2 | 4 | 6 | 16 | 241 | 25.2 | 8.78 |
| | isomerase OS = Glycine max | | | | | | | | |
| | PE = 3 SV = 1 - | | | | | | | | |
| | [K7LSN8_SOYBN] | | | | | | | | |
| O23961 | Peroxidase OS = Glycine max | 2.54 | 3 | 1 | 1 | 1 | 354 | 38.6 | 7.84 |
| | GN = GMIPER1 PE = 2 SV = 1 - | | | | | | | | |
| | [O23961_SOYBN] | | | | | | | | |
| B0M197 | Peroxisomal voltage-dependent | 20.29 | 5 | 2 | 4 | 4 | 276 | 29.8 | 8.65 |
| | anion-selective channel protein | | | | | | | | |
| | OS = Glycine max PE = 2 SV = 1 - | | | | | | | | |
| | [B0M197_SOYBN] | | | | | | | | |
| Q8W198 | Phosphate transporter | 9.94 | 7 | 2 | 2 | 2 | 342 | 36.3 | 9.04 |
| | OS = Glycine max PE = 2 SV = 1 - | | | | | | | | |
| | [Q8W198_SOYBN] | | | | | | | | |
| I1KDL4 | Phosphoenolpyruvate | 9.93 | 7 | 5 | 8 | 8 | 967 | 110.6 | 6.05 |
| | calboxylase OS = Glycine max | | | | | | | | |
| | GN = Gmppc2 PE = 2 SV = 1 - | | | | | | | | |
| | [I1KDL4_SOYBN] | | | | | | | | |
| C6T920 | Phosphoglycerate kinase | 49.79 | 1 | 1 | 9 | 11 | 233 | 25.3 | 9.32 |
| | (Fragment) OS = Glycine max | | | | | | | | |
| | PE = 2 SV = 1 - | | | | | | | | |
| | [C6T920_SOYBN] | | | | | | | | |
| I1KTX8 | Phosphoglycerate kinase | 27.84 | 1 | 1 | 10 | 13 | 485 | 50.2 | 8.13 |
| | OS = Glycine max PE = 3 SV = 1 - | | | | | | | | |
| | [I1KTX8_SOYBN] | | | | | | | | |
| I1MJC6 | Phosphoglycerate kinase | 23.87 | 1 | 1 | 6 | 8 | 377 | 39.0 | 8.65 |
| | OS = Glycine max PE = 3 SV = 1 - | | | | | | | | |
| | [I1MJC6_SOYBN] | | | | | | | | |
| I1MJC7 | Phosphoglycerate kinase | 49.38 | 1 | 6 | 15 | 19 | 401 | 42.4 | 6.32 |
| | OS = Glycine max PE = 3 SV = 1 - | | | | | | | | |
| | [I1MJC7_SOYBN] | | | | | | | | |
| I1KVD8 | Phospholipase D OS = Glycine | 18.29 | 6 | 11 | 11 | 12 | 809 | 91.6 | 5.69 |
| | max PE = 3 SV = 1 - | | | | | | | | |
| | [I1KVD8_SOYBN] | | | | | | | | |
| C6T8C4 | Phosphomannomutase | 4.45 | 2 | 1 | 1 | 1 | 247 | 28.0 | 6.24 |
| | OS = Glycine max PE = 2 SV = 1 - | | | | | | | | |
| | [C6T8C4_SOYBN] | | | | | | | | |
| C6TL29 | Phosphoribulokinase | 3.19 | 3 | 1 | 1 | 1 | 407 | 45.3 | 6.28 |
| | OS = Glycine max PE = 2 SV = 1 - | | | | | | | | |
| | [C6TL29_SOYBN] | | | | | | | | |
| Q2PMT9 | Photosystem II CP43 reaction | 4.65 | 1 | 2 | 2 | 2 | 473 | 51.9 | 7.21 |
| | center protein OS = Glycine max | | | | | | | | |
| | GN = psbC PE = 3 SV = 1 - | | | | | | | | |
| | [PSBC_SOYBN] | | | | | | | | |
| Q2PMT8 | Photosystem II D2 protein | 7.65 | 2 | 2 | 2 | 2 | 353 | 39.5 | 5.55 |
| | OS = Glycine max GN = psbD | | | | | | | | |
| | PE = 3 SV = 1 - [PSBD_SOYBN] | | | | | | | | |
| Q9SWB4 | Poly [ADP-ribose] polymerase | 20.25 | 2 | 4 | 14 | 16 | 815 | 91.6 | 5.59 |
| | 3 OS = Glycine max GN = PARP3 | | | | | | | | |
| | PE = 2 SV = 1 - | | | | | | | | |
| | [PARP3_SOYBN] | | | | | | | | |
| I1MXM6 | Polyadenylate-binding protein | 3.36 | 7 | 1 | 2 | 2 | 654 | 71.9 | 7.15 |
| | OS = Glycine max PE = 3 SV = 1 - | | | | | | | | |
| | [I1MXM6_SOYBN] | | | | | | | | |
| Q7XXT2 | Prepro beta-conglycinin alpha | 51.53 | 4 | 6 | 33 | 184 | 621 | 72.2 | 5.72 |
| | prime subunit OS = Glycine max | | | | | | | | |
| | PE = 1 SV = 1 - | | | | | | | | |
| | [Q7XXT2_SOYBN] | | | | | | | | |
| A7XZJ7 | Profilin OS = Glycine max PE = 2 | 17.56 | 7 | 1 | 2 | 4 | 131 | 14.1 | 4.84 |
| | SV = 1 - [A7XZJ7_SOYBN] | | | | | | | | |
| C6SVT2 | Profilin OS = Glycine max PE = 2 | 38.93 | 10 | 2 | 3 | 5 | 131 | 14.0 | 4.98 |
| | SV = 1 - [C6SVT2_SOYBN] | | | | | | | | |
| I1ME45 | Proline iminopeptidase | 2.05 | 1 | 1 | 1 | 1 | 391 | 44.4 | 6.43 |
| | OS = Glycine max PE = 3 SV = 1 - | | | | | | | | |
| | [I1ME45_SOYBN] | | | | | | | | |
| C6TAW4 | Proteasome subunit alpha type | 22.80 | 5 | 2 | 5 | 5 | 250 | 27.5 | 7.09 |
| | OS = Glycine max PE = 2 SV = 1 - | | | | | | | | |
| | [C6TAW4_SOYBN] | | | | | | | | |
| C6TAZ5 | Proteasome subunit alpha type | 17.60 | 1 | 1 | 4 | 4 | 250 | 27.2 | 7.80 |
| | OS = Glycine max PE = 2 SV = 1 - | | | | | | | | |
| | [C6TAZ5_SOYBN] | | | | | | | | |
| C6TGU2 | Proteasome subunit alpha type | 30.80 | 7 | 5 | 5 | 5 | 237 | 26.0 | 4.75 |
| | OS = Glycine max PE = 2 SV = 1 - | | | | | | | | |
| | [C6TGU2_SOYBN] | | | | | | | | |
| I1KTZ4 | Proteasome subunit alpha type | 19.21 | 10 | 3 | 3 | 3 | 177 | 19.2 | 9.45 |
| | OS = Glycine max PE = 3 SV = 1 - | | | | | | | | |
| | [I1KTZ4_SOYBN] | | | | | | | | |
| I1LSZ7 | Proteasome subunit alpha type | 22.36 | 5 | 5 | 5 | 6 | 246 | 27.4 | 5.86 |
| | OS = Glycine max PE = 3 SV = 1 - | | | | | | | | |
| | [I1LSZ7_SOYBN] | | | | | | | | |
| I1MN39 | Proteasome subunit alpha type | 15.26 | 1 | 3 | 3 | 3 | 249 | 27.2 | 7.44 |
| | OS = Glycine max PE = 3 SV = 1 - | | | | | | | | |
| | [I1MN39_SOYBN] | | | | | | | | |
| I1MXX5 | Proteasome subunit alpha type | 20.42 | 4 | 5 | 5 | 5 | 284 | 31.0 | 5.19 |
| | OS = Glycine max PE = 3 SV = 1 - | | | | | | | | |
| | [I1MXX5_SOYBN] | | | | | | | | |
| I1N783 | Proteasome subunit alpha type | 8.03 | 4 | 2 | 2 | 3 | 249 | 27.3 | 5.87 |
| | OS = Glycine max PE = 3 SV = 1 - | | | | | | | | |
| | [I1N783_SOYBN] | | | | | | | | |
| C6SVE5 | Proteasome subunit beta type | 20.60 | 2 | 4 | 4 | 5 | 233 | 25.1 | 5.55 |
| | OS = Glycine max PE = 2 SV = 1 - | | | | | | | | |
| | [C6SVE5_SOYBN] | | | | | | | | |
| C6TLJ9 | Proteasome subunit beta type | 2.94 | 3 | 1 | 1 | 1 | 272 | 29.2 | 6.14 |
| | OS = Glycine max PE = 2 SV = 1 - | | | | | | | | |
| | [C6TLJ9_SOYBN] | | | | | | | | |
| I1JJI5 | Proteasome subunit beta type | 4.52 | 3 | 1 | 1 | 1 | 199 | 21.4 | 7.83 |
| | OS = Glycine max PE = 3 SV = 1 - | | | | | | | | |
| | [I1JJI5_SOYBN] | | | | | | | | |
| I1MA65 | Proteasome subunit beta type | 4.71 | 4 | 1 | 1 | 1 | 191 | 21.3 | 5.60 |
| | OS = Glycine max PE = 3 SV = 1 - | | | | | | | | |
| | [I1MA65_SOYBN] | | | | | | | | |
| I1MQL3 | Protein argonaute OS = Glycine | 1.52 | 6 | 2 | 2 | 2 | 1053 | 116.4 | 9.31 |
| | max PE = 3 SV = 1 - | | | | | | | | |
| | [I1MQL3_SOYBN] | | | | | | | | |
| I7FST9 | Protein disulfide-isomerase | 61.14 | 11 | 6 | 22 | 40 | 525 | 58.7 | 5.17 |
| | OS = Glycine max GN = Gm | | | | | | | | |
| | pdil-1 PE = 2 SV = 1 - | | | | | | | | |
| | [I7FST9_SOYBN] | | | | | | | | |
| I1KAB7 | Protein disulfide-isomerase | 63.02 | 9 | 7 | 23 | 45 | 503 | 56.1 | 4.98 |
| | OS = Glycine max PE = 3 SV = 1 - | | | | | | | | |
| | [I1KAB7_SOYBN] | | | | | | | | |
| I1LL59 | Protein disulfide-isomerase | 4.95 | 2 | 3 | 3 | 3 | 586 | 64.8 | 4.79 |
| | OS = Glycine max PE = 3 SV = 1 - | | | | | | | | |
| | [I1LL59_SOYBN] | | | | | | | | |
| I1M4K9 | Protein disulfide-isomerase | 5.56 | 2 | 3 | 3 | 4 | 558 | 62.3 | 4.87 |
| | OS = Glycine max PE = 3 SV = 1 - | | | | | | | | |
| | [I1M4K9_SOYBN] | | | | | | | | |
| I1M5C5 | Protein ROOT HAIR | 1.37 | 4 | 1 | 1 | 1 | 802 | 89.2 | 5.68 |
| | DEFECTIVE 3 homolog | | | | | | | | |
| | OS = Glycine max PE = 3 SV = 2 - | | | | | | | | |
| | [I1M5C5_SOYBN] | | | | | | | | |
| I1N2Z5 | Protein SLE1 OS = Glycine max | 37.50 | 2 | 4 | 4 | 4 | 112 | 12.2 | 5.38 |
| | GN = SLE1 PE = 2 SV = 1 - | | | | | | | | |
| | [SLE1_SOYBN] | | | | | | | | |
| I1JLC8 | Protein SLE2 OS = Glycine max | 24.76 | 1 | 1 | 2 | 3 | 105 | 11.5 | 5.60 |
| | GN = SLE2 PE = 2 SV = 1 - | | | | | | | | |
| | [SLE2_SOYBN] | | | | | | | | |
| C6T0L2 | Protein SLE3 OS = Glycine max | 36.63 | 1 | 2 | 3 | 4 | 101 | 11.1 | 6.80 |
| | GN = SLE3 PE = 2 SV = 1 - | | | | | | | | |
| | [SLE3_SOYBN] | | | | | | | | |
| B1ACD3 | Putative Kunitz trypsin protease | 16.36 | 2 | 3 | 3 | 3 | 214 | 23.6 | 5.38 |
| | inhibitor OS = Glycine max | | | | | | | | |
| | PE = 2 SV = 1 - | | | | | | | | |
| | [B1ACD3_SOYBN] | | | | | | | | |
| Q9LDM8 | Putative pre-pro-subtilisin | 10.91 | 3 | 6 | 6 | 6 | 770 | 82.6 | 7.28 |
| | OS = Glycine max GN = scs1 | | | | | | | | |
| | PE = 2 SV = 1 - | | | | | | | | |
| | [Q9LDM8_SOYBN] | | | | | | | | |
| C6F117 | Putative ribosomal protein S15 | 12.31 | 3 | 2 | 2 | 2 | 130 | 14.8 | 9.89 |
| | OS = Glycine max PE = 2 SV = 1 - | | | | | | | | |
| | [C6F117_SOYBN] | | | | | | | | |
| G3E7M5 | Putative rubisco subunit | 15.58 | 4 | 7 | 7 | 8 | 584 | 61.7 | 5.40 |
| | binding-protein alpha subunit | | | | | | | | |
| | OS = Glycine max PE = 2 SV = 1 - | | | | | | | | |
| | [G3E7M5_SOYBN] | | | | | | | | |
| C6SW87 | Putative uncharacterized protein | 16.32 | 4 | 1 | 2 | 2 | 190 | 22.0 | 9.77 |
| | (Fragment) OS = Glycine max | | | | | | | | |
| | PE = 2 SV = 1 - | | | | | | | | |
| | [C6SW87_SOYBN] | | | | | | | | |
| C6SZN9 | Putative uncharacterized protein | 3.32 | 4 | 1 | 1 | 1 | 211 | 22.8 | 9.07 |
| | (Fragment) OS = Glycine max | | | | | | | | |
| | PE = 2 SV = 1 | | | | | | | | |
| | [C6SZN9_SOYBN] | | | | | | | | |
| C6SZP9 | Putative uncharacterized protein | 5.30 | 2 | 1 | 1 | 1 | 151 | 17.4 | 9.31 |
| | (Fragment) OS = Glycine max | | | | | | | | |
| | PE = 2 SV = 1 - | | | | | | | | |
| | [C6SZP9_SOYBN] | | | | | | | | |
| C6T0J3 | Putative uncharacterized protein | 4.86 | 2 | 1 | 1 | 1 | 247 | 26.4 | 6.58 |
| | (Fragment) OS = Glycine max | | | | | | | | |
| | PE = 2 SV = 1 - | | | | | | | | |
| | [C6T0J3_SOYBN] | | | | | | | | |
| C6T6Y9 | Putative uncharacterized protein | 3.74 | 6 | 1 | 1 | 1 | 187 | 20.5 | 4.68 |
| | (Fragment) OS = Glycine max | | | | | | | | |
| | PE = 2 SV = 1 - | | | | | | | | |
| | [C6T6Y9_SOYBN] | | | | | | | | |
| C6T7C4 | Putative uncharacterized protein | 11.02 | 10 | 2 | 2 | 2 | 245 | 26.0 | 5.83 |
| | (Fragment) OS = Glycine max | | | | | | | | |
| | PE = 2 SV = 1 - | | | | | | | | |
| | [C6T7C4_SOYBN] | | | | | | | | |
| C6T952 | Putative uncharacterized protein | 11.37 | 2 | 1 | 1 | 1 | 211 | 22.8 | 9.57 |
| | (Fragment) OS = Glycine max | | | | | | | | |
| | PE = 2 SV = 1 - | | | | | | | | |
| | [C6T952_SOYBN] | | | | | | | | |
| C6TA81 | Putative uncharacterized protein | 9.31 | 6 | 2 | 2 | 2 | 204 | 21.5 | 9.32 |
| | (Fragment) OS = Glycine max | | | | | | | | |
| | PE = 2 SV = 1 - | | | | | | | | |
| | [C6TA81_SOYBN] | | | | | | | | |
| C6TD18 | Putative uncharacterized protein | 14.52 | 2 | 1 | 3 | 3 | 241 | 26.2 | 6.64 |
| | (Fragment) OS = Glycine max | | | | | | | | |
| | PE = 2 SV = 1 - | | | | | | | | |
| | [C6TD18_SOYBN] | | | | | | | | |
| C6TGC7 | Putative uncharacterized protein | 7.63 | 9 | 1 | 1 | 1 | 262 | 28.6 | 7.33 |
| | (Fragment) OS = Glycine max | | | | | | | | |
| | PE = 2 SV = 1 - | | | | | | | | |
| | [C6TGC7_SOYBN] | | | | | | | | |
| C6TGH1 | Putative uncharacterized protein | 2.36 | 4 | 1 | 1 | 1 | 339 | 38.4 | 5.35 |
| | (Fragment) OS = Glycine max | | | | | | | | |
| | PE = 2 SV = 1 - | | | | | | | | |
| | [C6TGH1_SOYBN] | | | | | | | | |
| C6TIJ2 | Putative uncharacterized protein | 9.57 | 14 | 1 | 2 | 3 | 303 | 34.0 | 9.29 |
| | (Fragment) OS = Glycine max | | | | | | | | |
| | PE = 2 SV = 1 - | | | | | | | | |
| | [C6TIJ2_SOYBN] | | | | | | | | |
| C6TIW2 | Putative uncharacterized protein | 5.75 | 2 | 1 | 1 | 1 | 226 | 25.1 | 7.24 |
| | (Fragment) OS = Glycine max | | | | | | | | |
| | PE = 2 SV = 1 - | | | | | | | | |
| | [C6TIW2_SOYBN] | | | | | | | | |
| C6TNT7 | Putative uncharacterized protein | 6.57 | 3 | 1 | 1 | 1 | 213 | 23.3 | 9.10 |
| | (Fragment) OS = Glycine max | | | | | | | | |
| | PE = 2 SV = 1 - | | | | | | | | |
| | [C6TNT7_SOYBN] | | | | | | | | |
| C6SV69 | Putative uncharacterized protein | 15.19 | 4 | 1 | 2 | 2 | 158 | 17.8 | 7.20 |
| | OS = Glycine max PE = 2 SV = 1 - | | | | | | | | |
| | [C6SV69_SOYBN] | | | | | | | | |
| C6SVA6 | Putative uncharacterized protein | 8.57 | 4 | 1 | 1 | 2 | 105 | 12.0 | 10.37 |
| | OS = Glycine max PE = 2 SV = 1 - | | | | | | | | |
| | [C6SVA6_SOYBN] | | | | | | | | |
| C6SVH1 | Putative uncharacterized protein | 35.66 | 6 | 1 | 5 | 5 | 143 | 15.9 | 10.13 |
| | OS = Glycine max PE = 2 SV = 1 - | | | | | | | | |
| | [C6SVH1_SOYBN] | | | | | | | | |
| C6SVH8 | Putative uncharacterized protein | 10.43 | 5 | 1 | 1 | 1 | 115 | 12.1 | 7.12 |
| | OS = Glycine max PE = 2 SV = 1 - | | | | | | | | |
| | [C6SVH8_SOYBN] | | | | | | | | |
| C6SVJ6 | Putative uncharacterized protein | 6.52 | 2 | 1 | 1 | 1 | 184 | 20.6 | 9.32 |
| | OS = Glycine max PE = 2 SV = 1 - | | | | | | | | |
| | [C6SVJ6_SOYBN] | | | | | | | | |
| C6SVM2 | Putative uncharacterized protein | 39.16 | 2 | 4 | 4 | 9 | 166 | 17.4 | 8.94 |
| | OS = Glycine max PE = 2 SV = 1 - | | | | | | | | |
| | [C6SVM2_SOYBN] | | | | | | | | |
| C6SVR5 | Putative uncharacterized protein | 37.91 | 3 | 8 | 8 | 9 | 211 | 23.9 | 5.97 |
| | OS = Glycine max PE = 2 SV = 1 - | | | | | | | | |
| | [C6SVR5_SOYBN] | | | | | | | | |
| C6SVS3 | Putative uncharacterized protein | 13.25 | 2 | 1 | 1 | 2 | 151 | 15.9 | 5.10 |
| | OS = Glycine max PE = 2 SV = 1 - | | | | | | | | |
| | [C6SVS3_SOYBN] | | | | | | | | |
| C6SVS8 | Putative uncharacterized protein | 21.60 | 7 | 1 | 2 | 2 | 125 | 14.1 | 9.55 |
| | OS = Glycine max PE = 2 SV = 1 - | | | | | | | | |
| | [C6SVS8_SOYBN] | | | | | | | | |
| C6SVU2 | Putative uncharacterized protein | 13.64 | 2 | 1 | 1 | 1 | 88 | 9.5 | 8.88 |
| | OS = Glycine max PE = 2 SV = 1 - | | | | | | | | |
| | [C6SVU2_SOYBN] | | | | | | | | |
| C6SW15 | Putative uncharacterized protein | 53.39 | 8 | 14 | 14 | 16 | 236 | 26.3 | 9.58 |
| | OS = Glycine max PE = 2 SV = 1 - | | | | | | | | |
| | [C6SW15_SOYBN] | | | | | | | | |
| C6SW37 | Putative uncharacterized protein | 12.50 | 6 | 1 | 1 | 1 | 64 | 7.1 | 9.54 |
| | OS = Glycine max PE = 2 SV = 1 - | | | | | | | | |
| | [C6SW37_SOYBN] | | | | | | | | |
| C6SW41 | Putative uncharacterized protein | 29.95 | 6 | 7 | 7 | 7 | 197 | 23.1 | 10.29 |
| | OS = Glycine max PE = 2 SV = 1 - | | | | | | | | |
| | [C6SW41_SOYBN] | | | | | | | | |
| C6SWD7 | Putative uncharacterized protein | 3.93 | 3 | 1 | 1 | 1 | 229 | 25.6 | 7.74 |
| | OS = Glycine max PE = 2 SV = 1 - | | | | | | | | |
| | [C6SWD7_SOYBN] | | | | | | | | |
| C6SWG6 | Putative uncharacterized protein | 17.21 | 4 | 2 | 2 | 2 | 122 | 14.1 | 8.76 |
| | OS = Glycine max PE = 2 SV = 1 - | | | | | | | | |
| | [C6SWG6_SOYBN] | | | | | | | | |
| C6SWM6 | Putative uncharacterized protein | 14.37 | 9 | 2 | 2 | 2 | 167 | 19.1 | 8.97 |
| | OS = Glycine max PE = 2 SV = 1 - | | | | | | | | |
| | [C6SWM6_SOYBN] | | | | | | | | |
| C6SWW0 | Putative uncharacterized protein | 13.64 | 5 | 1 | 1 | 1 | 88 | 9.6 | 4.41 |
| | OS = Glycine max PE = 2 SV = 1 - | | | | | | | | |
| | [C6SWW0_SOYBN] | | | | | | | | |
| C6SWZ2 | Putative uncharacterized protein | 9.76 | 11 | 1 | 2 | 2 | 205 | 23.4 | 5.22 |
| | OS = Glycine max PE = 2 SV = 1 - | | | | | | | | |
| | [C6SWZ2_SOYBN] | | | | | | | | |
| C6SX05 | Putative uncharacterized protein | 5.24 | 3 | 1 | 1 | 1 | 191 | 21.2 | 6.37 |
| | OS = Glycine max PE = 2 SV = 1 - | | | | | | | | |
| | [C6SX05_SOYBN] | | | | | | | | |
| C6SXA1 | Putative uncharacterized protein | 4.02 | 4 | 1 | 1 | 1 | 174 | 19.4 | 4.55 |
| | OS = Glycine max PE = 2 SV = 1 - | | | | | | | | |
| | [C6SXA1_SOYBN] | | | | | | | | |
| C6SXA3 | Putative uncharacterized protein | 40.63 | 3 | 3 | 8 | 12 | 192 | 21.6 | 9.51 |
| | OS = Glycine max PE = 2 SV = 1 - | | | | | | | | |
| | [C6SXA3_SOYBN] | | | | | | | | |
| C6SXE9 | Putative uncharacterized protein | 20.18 | 4 | 2 | 2 | 2 | 109 | 12.4 | 5.69 |
| | OS = Glycine max PE = 2 SV = 1 - | | | | | | | | |
| | [C6SXE9_SOYBN] | | | | | | | | |
| C6SXH8 | Putative uncharacterized protein | 29.49 | 5 | 6 | 6 | 7 | 234 | 25.9 | 7.77 |
| | OS = Glycine max PE = 2 SV = 1 - | | | | | | | | |
| | [C6SXH8_SOYBN] | | | | | | | | |
| C6SXP1 | Putative uncharacterized protein | 40.87 | 5 | 6 | 8 | 13 | 230 | 25.1 | 4.60 |
| | OS = Glycine max PE = 2 SV = 1 - | | | | | | | | |
| | [C6SXP1_SOYBN] | | | | | | | | |
| C6SXQ8 | Putative uncharacterized protein | 10.00 | 4 | 1 | 1 | 1 | 120 | 13.8 | 11.52 |
| | OS = Glycine max PE = 2 SV = 1 - | | | | | | | | |
| | [C6SXQ8_SOYBN] | | | | | | | | |
| C6SXS9 | Putative uncharacterized protein | 59.15 | 3 | 3 | 9 | 12 | 213 | 23.5 | 6.21 |
| | OS = Glycine max PE = 2 SV = 1 - | | | | | | | | |
| | [C6SXS9_SOYBN] | | | | | | | | |
| C6SXX0 | Putative uncharacterized protein | 4.58 | 2 | 1 | 1 | 1 | 262 | 28.1 | 8.15 |
| | OS = Glycine max PE = 2 SV = 1 - | | | | | | | | |
| | [C6SXX0_SOYBN] | | | | | | | | |
| C6SXX6 | Putative uncharacterized protein | 8.66 | 2 | 1 | 1 | 1 | 127 | 14.6 | 6.81 |
| | OS = Glycine max PE = 2 SV = 1 - | | | | | | | | |
| | [C6SXX6_SOYBN] | | | | | | | | |
| C6SY42 | Putative uncharacterized protein | 40.18 | 4 | 5 | 7 | 12 | 224 | 24.3 | 4.70 |
| | OS = Glycine max PE = 2 SV = 1 - | | | | | | | | |
| | [C6SY42_SOYBN] | | | | | | | | |
| C6SY44 | Putative uncharacterized protein | 5.58 | 1 | 1 | 1 | 1 | 197 | 21.8 | 9.03 |
| | OS = Glycine max PE = 2 SV = 1 - | | | | | | | | |
| | [C6SY44_SOYBN] | | | | | | | | |
| C6SYI8 | Putative uncharacterized protein | 28.05 | 6 | 3 | 5 | 6 | 164 | 18.4 | 6.34 |
| | OS = Glycine max PE = 2 SV = 1 - | | | | | | | | |
| | [C6SYI8_SOYBN] | | | | | | | | |
| C6SYJ3 | Putative uncharacterized protein | 13.51 | 2 | 1 | 1 | 1 | 74 | 8.4 | 9.16 |
| | OS = Glycine max PE = 2 SV = 1 - | | | | | | | | |
| | [C6SYJ3_SOYBN] | | | | | | | | |
| C6SYK1 | Putative uncharacterized protein | 5.29 | 2 | 1 | 1 | 1 | 170 | 19.4 | 4.96 |
| | OS = Glycine max PE = 2 SV = 1 - | | | | | | | | |
| | [C6SYK1_SOYBN] | | | | | | | | |
| C6SYQ4 | Putative uncharacterized protein | 3.45 | 2 | 1 | 1 | 1 | 174 | 19.4 | 4.42 |
| | OS = Glycine max PE = 2 SV = 1 - | | | | | | | | |
| | [C6SYQ4_SOYBN] | | | | | | | | |
| C6SZ00 | Putative uncharacterized protein | 12.27 | 9 | 2 | 2 | 4 | 163 | 18.3 | 10.71 |
| | OS = Glycine max PE = 2 SV = 1 - | | | | | | | | |
| | [C6SZ00_SOYBN] | | | | | | | | |
| C6SZ41 | Putative uncharacterized protein | 18.49 | 2 | 1 | 2 | 2 | 146 | 16.0 | 7.43 |
| | OS = Glycine max PE = 2 SV = 1 - | | | | | | | | |
| | [C6SZ41_SOYBN] | | | | | | | | |
| C6SZI6 | Putative uncharacterized protein | 13.04 | 3 | 1 | 2 | 3 | 184 | 21.2 | 6.06 |
| | OS = Glycine max PE = 2 SV = 1 - | | | | | | | | |
| | [C6SZI6_SOYBN] | | | | | | | | |
| C6SZV4 | Putative uncharacterized protein | 14.55 | 3 | 1 | 1 | 1 | 165 | 16.5 | 5.00 |
| | OS = Glycine max PE = 2 SV = 1 - | | | | | | | | |
| | [C6SZV4_SOYBN] | | | | | | | | |
| C6SZW0 | Putative uncharacterized protein | 23.65 | 8 | 3 | 3 | 3 | 203 | 22.1 | 4.59 |
| | OS = Glycine max PE = 2 SV = 1 - | | | | | | | | |
| | [C6SZW0_SOYBN] | | | | | | | | |
| C6T017 | Putative uncharacterized protein | 27.08 | 3 | 1 | 5 | 7 | 192 | 21.7 | 9.55 |
| | OS = Glycine max PE = 2 SV = 1 - | | | | | | | | |
| | [C6T017_SOYBN] | | | | | | | | |
| C6T049 | Putative uncharacterized protein | 6.02 | 2 | 1 | 1 | 1 | 166 | 18.0 | 5.76 |
| | OS = Glycine max PE = 2 SV = 1 - | | | | | | | | |
| | [C6T049_SOYBN] | | | | | | | | |
| C6T078 | Putative uncharacterized protein | 29.27 | 5 | 1 | 4 | 6 | 123 | 14.3 | 10.83 |
| | OS = Glycine max PE = 2 SV = 1 - | | | | | | | | |
| | [C6T078_SOYBN] | | | | | | | | |
| C6T0F7 | Putative uncharacterized protein | 7.88 | 2 | 1 | 1 | 1 | 203 | 22.1 | 4.61 |
| | OS = Glycine max PE = 2 SV = 1 - | | | | | | | | |
| | [C6T0F7_SOYBN] | | | | | | | | |
| C6T1Q7 | Putative uncharacterized protein | 59.35 | 2 | 9 | 9 | 23 | 155 | 17.8 | 6.38 |
| | OS = Glycine max PE = 2 SV = 1 - | | | | | | | | |
| | [C6T1Q7_SOYBN] | | | | | | | | |
| C6T1U4 | Putative uncharacterized protein | 19.35 | 3 | 1 | 1 | 1 | 62 | 7.0 | 9.57 |
| | OS = Glycine max PE = 2 SV = 1 - | | | | | | | | |
| | [C6T1U4_SOYBN] | | | | | | | | |
| C6T2C1 | Putative uncharacterized protein | 12.74 | 2 | 1 | 1 | 1 | 157 | 17.1 | 7.80 |
| | OS = Glycine max PE = 2 SV = 1 - | | | | | | | | |
| | [C6T2C1_SOYBN] | | | | | | | | |
| C6T2G4 | Putative uncharacterized protein | 8.67 | 3 | 1 | 1 | 1 | 173 | 19.7 | 9.63 |
| | OS = Glycine max PE = 2 SV = 1 - | | | | | | | | |
| | [C6T2G4_SOYBN] | | | | | | | | |
| C6T2W1 | Putative uncharacterized protein | 5.73 | 4 | 1 | 1 | 1 | 192 | 21.9 | 6.71 |
| | OS = Glycine max PE = 2 SV = 1 - | | | | | | | | |
| | [C6T2W1_SOYBN] | | | | | | | | |
| C6T2W7 | Putative uncharacterized protein | 5.91 | 2 | 1 | 1 | 1 | 220 | 25.3 | 5.69 |
| | OS = Glycine max PE = 2 SV = 1 - | | | | | | | | |
| | [C6T2W7_SOYBN] | | | | | | | | |
| C6T372 | Putative uncharacterized protein | 22.29 | 35 | 4 | 4 | 5 | 175 | 20.5 | 5.44 |
| | OS = Glycine max PE = 2 SV = 1 - | | | | | | | | |
| | [C6T372_SOYBN] | | | | | | | | |
| C6T3E0 | Putative uncharacterized protein | 8.87 | 4 | 2 | 2 | 2 | 248 | 27.6 | 5.71 |
| | OS = Glycine max PE = 2 SV = 1 - | | | | | | | | |
| | [C6T3E0_SOYBN] | | | | | | | | |
| C6T3K4 | Putative uncharacterized protein | 12.68 | 5 | 2 | 2 | 2 | 205 | 22.8 | 9.00 |
| | OS = Glycine max PE = 2 SV = 1 - | | | | | | | | |
| | [C6T3K4_SOYBN] | | | | | | | | |
| C6T3L6 | Putative uncharacterized protein | 11.76 | 2 | 2 | 2 | 2 | 170 | 19.3 | 4.93 |
| | OS = Glycine max PE = 2 SV = 1 - | | | | | | | | |
| | [C6T3L6_SOYBN] | | | | | | | | |
| C6T420 | Putative uncharacterized protein | 9.43 | 2 | 1 | 1 | 1 | 159 | 17.4 | 7.12 |
| | OS = Glycine max PE = 2 SV = 1 - | | | | | | | | |
| | [C6T420_SOYBN] | | | | | | | | |
| C6T441 | Putative uncharacterized protein | 18.02 | 20 | 2 | 2 | 2 | 111 | 12.7 | 7.24 |
| | OS = Glycine max PE = 2 SV = 1 - | | | | | | | | |
| | [C6T441_SOYBN] | | | | | | | | |
| C6T4E5 | Putative uncharacterized protein | 4.63 | 2 | 1 | 1 | 1 | 259 | 27.6 | 8.35 |
| | OS = Glycine max PE = 2 SV = 1 - | | | | | | | | |
| | [C6T4E5_SOYBN] | | | | | | | | |
| C6T4J5 | Putative uncharacterized protein | 6.83 | 1 | 1 | 1 | 2 | 249 | 25.2 | 6.95 |
| | OS = Glycine max PE = 2 SV = 1 - | | | | | | | | |
| | [C6T4J5_SOYBN] | | | | | | | | |
| C6T5M6 | Putative uncharacterized protein | 16.34 | 4 | 1 | 3 | 6 | 153 | 17.4 | 5.71 |
| | OS = Glycine max PE = 2 SV = 1 - | | | | | | | | |
| | [C6T5M6_SOYBN] | | | | | | | | |
| C6T5N1 | Putative uncharacterized protein | 35.54 | 5 | 5 | 5 | 5 | 166 | 17.8 | 8.97 |
| | OS = Glycine max PE = 2 SV = 1 - | | | | | | | | |
| | [C6T5N1_SOYBN] | | | | | | | | |
| C6T5Y6 | Putative uncharacterized protein | 4.08 | 3 | 1 | 1 | 1 | 196 | 20.7 | 8.31 |
| | OS = Glycine max PE = 2 SV = 1 - | | | | | | | | |
| | [C6T5Y6_SOYBN] | | | | | | | | |
| C6T7D9 | Putative uncharacterized protein | 19.04 | 4 | 6 | 6 | 6 | 436 | 47.5 | 6.35 |
| | OS = Glycine max PE = 2 SV = 1 - | | | | | | | | |
| | [C6T7D9_SOYBN] | | | | | | | | |
| C6T7J5 | Putative uncharacterized protein | 7.37 | 4 | 2 | 2 | 2 | 312 | 35.1 | 6.14 |
| | OS = Glycine max PE = 2 SV = 1 - | | | | | | | | |
| | [C6T7J5_SOYBN] | | | | | | | | |
| C6T828 | Putative uncharacterized protein | 5.35 | 2 | 1 | 1 | 1 | 299 | 32.5 | 5.55 |
| | OS = Glycine max PE = 2 SV = 1 - | | | | | | | | |
| | [C6T828_SOYBN] | | | | | | | | |
| C6T8B0 | Putative uncharacterized protein | 11.43 | 3 | 1 | 4 | 5 | 420 | 47.7 | 6.16 |
| | OS = Glycine max PE = 2 SV = 1 - | | | | | | | | |
| | [C6T8B0_SOYBN] | | | | | | | | |
| C6T8D7 | Putative uncharacterized protein | 10.30 | 6 | 3 | 3 | 3 | 301 | 32.6 | 6.16 |
| | OS = Glycine max PE = 2 SV = 1 - | | | | | | | | |
| | [C6T8D7_SOYBN] | | | | | | | | |
| C6T8T9 | Putative uncharacterized protein | 6.35 | 3 | 1 | 1 | 1 | 189 | 20.9 | 8.38 |
| | OS = Glycine max PE = 2 SV = 1 - | | | | | | | | |
| | [C6T8T9_SOYBN] | | | | | | | | |
| C6T8W9 | Putative uncharacterized protein | 47.44 | 1 | 7 | 14 | 36 | 293 | 31.7 | 7.69 |
| | OS = Glycine max PE = 2 SV = 1 - | | | | | | | | |
| | [C6T8W9_SOYBN] | | | | | | | | |
| C6T8X3 | Putative uncharacterized protein | 20.34 | 9 | 2 | 8 | 9 | 413 | 46.9 | 5.68 |
| | OS = Glycine max PE = 2 SV = 1 - | | | | | | | | |
| | [C6T8X3_SOYBN] | | | | | | | | |
| C6T960 | Putative uncharacterized protein | 3.38 | 2 | 1 | 1 | 1 | 296 | 32.0 | 5.92 |
| | OS = Glycine max PE = 2 SV = 1 - | | | | | | | | |
| | [C6T960_SOYBN] | | | | | | | | |
| C6T995 | Putative uncharacterized protein | 3.24 | 5 | 1 | 1 | 1 | 247 | 27.1 | 7.96 |
| | OS = Glycine max PE = 2 SV = 1 - | | | | | | | | |
| | [C6T995_SOYBN] | | | | | | | | |
| C6T9C2 | Putative uncharacterized protein | 20.71 | 4 | 6 | 6 | 7 | 309 | 34.6 | 6.19 |
| | OS = Glycine max PE = 2 SV = 1 - | | | | | | | | |
| | [C6T9C2_SOYBN] | | | | | | | | |
| C6T9M0 | Putative uncharacterized protein | 3.15 | 2 | 1 | 2 | 2 | 476 | 52.3 | 7.01 |
| | OS = Glycine max PE = 2 SV = 1 - | | | | | | | | |
| | [C6T9M0_SOYBN] | | | | | | | | |
| C6T9X5 | Putative uncharacterized protein | 18.75 | 2 | 7 | 7 | 8 | 400 | 43.6 | 6.14 |
| | OS = Glycine max PE = 2 SV = 1 - | | | | | | | | |
| | [C6T9X5_SOYBN] | | | | | | | | |
| C6TA68 | Putative uncharacterized protein | 13.29 | 6 | 1 | 2 | 4 | 173 | 19.6 | 9.29 |
| | OS = Glycine max PE = 2 SV = 1 - | | | | | | | | |
| | [C6TA68_SOYBN] | | | | | | | | |
| C6TAE6 | Putative uncharacterized protein | 6.97 | 3 | 1 | 1 | 1 | 201 | 22.2 | 4.68 |
| | OS = Glycine max PE = 2 SV = 1 - | | | | | | | | |
| | [C6TAE6_SOYBN] | | | | | | | | |
| C6TAN1 | Putative uncharacterized protein | 22.70 | 16 | 7 | 8 | 10 | 423 | 47.4 | 5.11 |
| | OS = Glycine max PE = 2 SV = 1 - | | | | | | | | |
| | [C6TAN1_SOYBN] | | | | | | | | |
| C6TAV6 | Putative uncharacterized protein | 3.98 | 2 | 1 | 1 | 1 | 352 | 40.3 | 7.93 |
| | OS = Glycine max PE = 2 SV = 1 - | | | | | | | | |
| | [C6TAV6_SOYBN] | | | | | | | | |
| C6TAX0 | Putative uncharacterized protein | 6.63 | 6 | 2 | 2 | 2 | 362 | 39.8 | 5.91 |
| | OS = Glycine max PE = 2 SV = 1 - | | | | | | | | |
| | [C6TAX0_SOYBN] | | | | | | | | |
| C6TAZ2 | Putative uncharacterized protein | 23.70 | 7 | 4 | 4 | 4 | 211 | 23.2 | 7.42 |
| | OS = Glycine max PE = 2 SV = 1 - | | | | | | | | |
| | [C6TAZ2_SOYBN] | | | | | | | | |
| C6TB70 | Putative uncharacterized protein | 55.50 | 4 | 9 | 9 | 13 | 218 | 24.4 | 6.92 |
| | OS = Glycine max PE = 2 SV = 1 - | | | | | | | | |
| | [C6TB70_SOYBN] | | | | | | | | |
| C6TB80 | Putative uncharacterized protein | 30.15 | 2 | 4 | 4 | 5 | 136 | 14.6 | 6.93 |
| | OS = Glycine max PE = 2 SV = 1 - | | | | | | | | |
| | [C6TB80_SOYBN] | | | | | | | | |
| C6TBG9 | Putative uncharacterized protein | 14.66 | 4 | 1 | 3 | 3 | 341 | 37.6 | 5.72 |
| | OS = Glycine max PE = 2 SV = 1 - | | | | | | | | |
| | [C6TBG9_SOYBN] | | | | | | | | |
| C6TBW6 | Putative uncharacterized protein | 6.72 | 3 | 1 | 1 | 1 | 119 | 11.9 | 4.56 |
| | OS = Glycine max PE = 2 SV = 1 - | | | | | | | | |
| | [C6TBW6_SOYBN] | | | | | | | | |
| C6TBW8 | Putative uncharacterized protein | 2.91 | 4 | 1 | 1 | 1 | 344 | 37.4 | 6.95 |
| | OS = Glycine max PE = 2 SV = 1 - | | | | | | | | |
| | [C6TBW8_SOYBN] | | | | | | | | |
| C6TC20 | Putative uncharacterized protein | 2.68 | 3 | 1 | 1 | 1 | 336 | 36.3 | 8.15 |
| | OS = Glycine max PE = 2 SV = 1 - | | | | | | | | |
| | [C6TC20_SOYBN] | | | | | | | | |
| C6TCC4 | Putative uncharacterized protein | 30.97 | 4 | 1 | 3 | 3 | 155 | 17.5 | 10.40 |
| | OS = Glycine max PE = 2 SV = 1 - | | | | | | | | |
| | [C6TCC4_SOYBN] | | | | | | | | |
| C6TCF1 | Putative uncharacterized protein | 23.65 | 2 | 4 | 4 | 7 | 241 | 27.8 | 5.40 |
| | OS = Glycine max PE = 2 SV = 1 - | | | | | | | | |
| | [C6TCF1_SOYBN] | | | | | | | | |
| C6TCG3 | Putative uncharacterized protein | 4.89 | 4 | 1 | 1 | 1 | 184 | 20.3 | 5.47 |
| | OS = Glycine max PE = 2 SV = 1 - | | | | | | | | |
| | [C6TCG3_SOYBN] | | | | | | | | |
| C6TCH6 | Putative uncharacterized protein | 11.85 | 3 | 1 | 2 | 3 | 287 | 30.9 | 6.80 |
| | OS = Glycine max PE = 2 SV = 1 - | | | | | | | | |
| | [C6TCH6_SOYBN] | | | | | | | | |
| C6TCH9 | Putative uncharacterized protein | 10.41 | 3 | 2 | 2 | 2 | 269 | 28.3 | 6.79 |
| | OS = Glycine max PE = 2 SV = 1 - | | | | | | | | |
| | [C6TCH9_SOYBN] | | | | | | | | |
| C6TCR6 | Putative uncharacterized protein | 10.77 | 3 | 3 | 3 | 4 | 325 | 36.2 | 5.07 |
| | OS = Glycine max PE = 2 SV = 1 - | | | | | | | | |
| | [C6TCR6_SOYBN] | | | | | | | | |
| C6TCS7 | Putative uncharacterized protein | 3.72 | 5 | 1 | 1 | 1 | 376 | 42.6 | 6.70 |
| | OS = Glycine max PE = 2 SV = 1 - | | | | | | | | |
| | [C6TCS7_SOYBN] | | | | | | | | |
| C6TCU1 | Putative uncharacterized protein | 11.20 | 7 | 1 | 2 | 2 | 259 | 29.6 | 4.88 |
| | OS = Glycine max PE = 2 SV = 1 - | | | | | | | | |
| | [C6TCU1_SOYBN] | | | | | | | | |
| C6TCX8 | Putative uncharacterized protein | 6.07 | 4 | 2 | 2 | 2 | 478 | 51.7 | 6.80 |
| | OS = Glycine max PE = 2 SV = 1 - | | | | | | | | |
| | [C6TCX8_SOYBN] | | | | | | | | |
| C6TD06 | Putative uncharacterized protein | 5.49 | 4 | 1 | 1 | 1 | 273 | 28.9 | 8.27 |
| | OS = Glycine max PE = 2 SV = 1 - | | | | | | | | |
| | [C6TD06_SOYBN] | | | | | | | | |
| C6TDC1 | Putative uncharacterized protein | 9.41 | 5 | 2 | 2 | 2 | 170 | 18.1 | 4.84 |
| | OS = Glycine max PE = 2 SV = 1 - | | | | | | | | |
| | [C6TDC1_SOYBN] | | | | | | | | |
| C6TDL5 | Putative uncharacterized protein | 45.77 | 7 | 9 | 9 | 14 | 260 | 28.1 | 10.71 |
| | OS = Glycine max PE = 2 SV = 1 - | | | | | | | | |
| | [C6TDL5_SOYBN] | | | | | | | | |
| C6TDP7 | Putative uncharacterized protein | 3.41 | 2 | 1 | 1 | 1 | 323 | 35.8 | 5.68 |
| | OS = Glycine max PE = 2 SV = 1 - | | | | | | | | |
| | [C6TDP7_SOYBN] | | | | | | | | |
| C6TDP9 | Putative uncharacterized protein | 13.00 | 2 | 3 | 3 | 3 | 300 | 32.7 | 5.52 |
| | OS = Glycine max PE = 2 SV = 1 - | | | | | | | | |
| | [C6TDP9_SOYBN] | | | | | | | | |
| C6TEI8 | Putative uncharacterized protein | 7.52 | 5 | 1 | 1 | 1 | 133 | 14.7 | 7.47 |
| | OS = Glycine max PE = 2 SV = 1 - | | | | | | | | |
| | [C6TEI8_SOYBN] | | | | | | | | |
| C6TEV7 | Putative uncharacterized protein | 15.12 | 6 | 2 | 3 | 3 | 291 | 31.7 | 7.74 |
| | OS = Glycine max PE = 2 SV = 1 - | | | | | | | | |
| | [C6TEV7_SOYBN] | | | | | | | | |
| C6TEW4 | Putative uncharacterized protein | 9.17 | 2 | 1 | 1 | 1 | 109 | 10.3 | 9.76 |
| | OS = Glycine max PE = 2 SV = 1 - | | | | | | | | |
| | [C6TEW4_SOYBN] | | | | | | | | |
| C6TFB9 | Putative uncharacterized protein | 5.49 | 3 | 1 | 1 | 1 | 164 | 18.2 | 10.56 |
| | OS = Glycine max PE = 2 SV = 1 - | | | | | | | | |
| | [C6TFB9_SOYBN] | | | | | | | | |
| C6TG28 | Putative uncharacterized protein | 3.16 | 3 | 1 | 1 | 1 | 348 | 38.9 | 7.02 |
| | OS = Glycine max PE = 2 SV = 1 - | | | | | | | | |
| | [C6TG28_SOYBN] | | | | | | | | |
| C6TG44 | Putative uncharacterized protein | 9.12 | 4 | 2 | 2 | 2 | 340 | 38.4 | 5.16 |
| | OS = Glycine max PE = 2 SV = 1 - | | | | | | | | |
| | [C6TG44_SOYBN] | | | | | | | | |
| C6TG59 | Putative uncharacterized protein | 14.22 | 3 | 5 | 5 | 5 | 464 | 49.7 | 5.68 |
| | OS = Glycine max PE = 2 SV = 1 - | | | | | | | | |
| | [C6TG59_SOYBN] | | | | | | | | |
| C6TGM9 | Putative uncharacterized protein | 15.27 | 3 | 1 | 2 | 2 | 203 | 22.3 | 6.38 |
| | OS = Glycine max PE = 2 SV = 1 - | | | | | | | | |
| | [C6TGM9_SOYBN] | | | | | | | | |
| C6TGR0 | Putative uncharacterized protein | 4.66 | 3 | 1 | 1 | 1 | 365 | 39.9 | 6.09 |
| | OS = Glycine max PE = 2 SV = 1 - | | | | | | | | |
| | [C6TGR0_SOYBN] | | | | | | | | |
| C6TGR4 | Putative uncharacterized protein | 5.31 | 2 | 1 | 1 | 1 | 245 | 26.9 | 8.85 |
| | OS = Glycine max PE = 2 SV = 1 - | | | | | | | | |
| | [C6TGR4_SOYBN] | | | | | | | | |
| C6TGR8 | Putative uncharacterized protein | 10.21 | 4 | 3 | 3 | 3 | 421 | 45.8 | 6.40 |
| | OS = Glycine max PE = 2 SV = 1 - | | | | | | | | |
| | [C6TGR8_SOYBN] | | | | | | | | |
| C6TGT2 | Putative uncharacterized protein | 3.45 | 6 | 1 | 1 | 1 | 203 | 22.7 | 7.58 |
| | OS = Glycine max PE = 2 SV = 1 - | | | | | | | | |
| | [C6TGT2_SOYBN] | | | | | | | | |
| C6TGZ9 | Putative uncharacterized protein | 39.79 | 9 | 3 | 12 | 15 | 377 | 41.8 | 5.40 |
| | OS = Glycine max PE = 2 SV = 1 - | | | | | | | | |
| | [C6TGZ9_SOYBN] | | | | | | | | |
| C6TH12 | Putative uncharacterized protein | 19.91 | 1 | 3 | 7 | 10 | 457 | 51.5 | 4.70 |
| | OS = Glycine max PE = 2 SV = 1 - | | | | | | | | |
| | [C6TH12_SOYBN] | | | | | | | | |
| C6TH44 | Putative uncharacterized protein | 18.69 | 7 | 4 | 4 | 5 | 289 | 31.8 | 9.72 |
| | OS = Glycine max PE = 2 SV = 1 - | | | | | | | | |
| | [C6TH44_SOYBN] | | | | | | | | |
| C6THC0 | Putative uncharacterized protein | 4.24 | 2 | 1 | 1 | 1 | 283 | 32.1 | 6.32 |
| | OS = Glycine max PE = 2 SV = 1 - | | | | | | | | |
| | [C6THC0_SOYBN] | | | | | | | | |
| C6TI26 | Putative uncharacterized protein | 19.13 | 9 | 1 | 7 | 8 | 413 | 46.6 | 5.57 |
| | OS = Glycine max PE = 2 SV = 1 - | | | | | | | | |
| | [C6TI26_SOYBN] | | | | | | | | |
| C6TI42 | Putative uncharacterized protein | 16.36 | 1 | 1 | 1 | 1 | 55 | 6.3 | 4.61 |
| | OS = Glycine max PE = 2 SV = 1 - | | | | | | | | |
| | [C6TI42_SOYBN] | | | | | | | | |
| C6TI56 | Putative uncharacterized protein | 2.64 | 2 | 1 | 1 | 1 | 341 | 37.4 | 5.44 |
| | OS = Glycine max PE = 2 SV = 1 - | | | | | | | | |
| | [C6TI56_SOYBN] | | | | | | | | |
| C6TIB8 | Putative uncharacterized protein | 6.87 | 4 | 2 | 2 | 2 | 466 | 49.6 | 5.94 |
| | OS = Glycine max PE = 2 SV = 1 - | | | | | | | | |
| | [C6TIB8_SOYBN] | | | | | | | | |
| C6TIC5 | Putative uncharacterized protein | 9.33 | 2 | 1 | 1 | 1 | 150 | 17.0 | 6.68 |
| | OS = Glycine max PE = 2 SV = 1 - | | | | | | | | |
| | [C6TIC5_SOYBN] | | | | | | | | |
| C6TID1 | Putative uncharacterized protein | 11.75 | 4 | 3 | 3 | 3 | 315 | 35.1 | 4.89 |
| | OS = Glycine max PE = 2 SV = 1 - | | | | | | | | |
| | [C6TID1_SOYBN] | | | | | | | | |
| C6TIR2 | Putative uncharacterized protein | 41.61 | 6 | 1 | 6 | 7 | 149 | 16.9 | 4.27 |
| | OS = Glycine max PE = 2 SV = 1 - | | | | | | | | |
| | [C6TIR2_SOYBN] | | | | | | | | |
| C6TIX4 | Putative uncharacterized protein | 29.18 | 12 | 6 | 6 | 6 | 305 | 34.4 | 6.13 |
| | OS = Glycine max PE = 2 SV = 1 - | | | | | | | | |
| | [C6TIX4_SOYBN] | | | | | | | | |
| C6TJ08 | Putative uncharacterized protein | 15.00 | 2 | 4 | 4 | 4 | 320 | 35.2 | 6.27 |
| | OS = Glycine max PE = 2 SV = 1 - | | | | | | | | |
| | [C6TJ08_SOYBN] | | | | | | | | |
| C6TJC4 | Putative uncharacterized protein | 6.11 | 3 | 1 | 1 | 1 | 311 | 34.8 | 7.17 |
| | OS = Glycine max PE = 2 SV = 1 - | | | | | | | | |
| | [C6TJC4_SOYBN] | | | | | | | | |
| C6TJH0 | Putative uncharacterized protein | 14.83 | 5 | 2 | 2 | 2 | 236 | 25.3 | 6.11 |
| | OS = Glycine max PE = 2 SV = 1 - | | | | | | | | |
| | [C6TJH0_SOYBN] | | | | | | | | |
| C6TJQ6 | Putative uncharacterized protein | 3.93 | 2 | 1 | 1 | 1 | 280 | 29.8 | 6.15 |
| | OS = Glycine max PE = 2 SV = 1 - | | | | | | | | |
| | [C6TJQ6_SOYBN] | | | | | | | | |
| C6TK38 | Putative uncharacterized protein | 13.22 | 3 | 4 | 5 | 8 | 484 | 53.4 | 6.09 |
| | OS = Glycine max PE = 2 SV = 1 - | | | | | | | | |
| | [C6TK38_SOYBN] | | | | | | | | |
| C6TK65 | Putative uncharacterized protein | 8.00 | 3 | 2 | 2 | 2 | 300 | 32.8 | 4.70 |
| | OS = Glycine max PE = 2 SV = 1 - | | | | | | | | |
| | [C6TK65_SOYBN] | | | | | | | | |
| C6TKC9 | Putative uncharacterized protein | 4.69 | 1 | 2 | 2 | 2 | 490 | 55.2 | 5.44 |
| | OS = Glycine max PE = 2 SV = 1 - | | | | | | | | |
| | [C6TKC9_SOYBN] | | | | | | | | |
| C6TKE6 | Putative uncharacterized protein | 6.92 | 1 | 1 | 2 | 3 | 289 | 30.7 | 6.00 |
| | OS = Glycine max PE = 2 SV = 1 - | | | | | | | | |
| | [C6TKE6_SOYBN] | | | | | | | | |
| C6TKJ1 | Putative uncharacterized protein | 2.70 | 4 | 1 | 1 | 1 | 407 | 45.6 | 7.36 |
| | OS = Glycine max PE = 2 SV = 1 - | | | | | | | | |
| | [C6TKJ1_SOYBN] | | | | | | | | |
| C6TKP4 | Putative uncharacterized protein | 4.31 | 1 | 1 | 1 | 4 | 255 | 27.1 | 7.06 |
| | OS = Glycine max PE = 2 SV = 1 - | | | | | | | | |
| | [C6TKP4_SOYBN] | | | | | | | | |
| C6TKQ1 | Putative uncharacterized protein | 4.83 | 3 | 1 | 1 | 1 | 269 | 29.4 | 6.54 |
| | OS = Glycine max PE = 2 SV = 1 - | | | | | | | | |
| | [C6TKQ1_SOYBN] | | | | | | | | |
| C6TKR5 | Putative uncharacterized protein | 9.49 | 4 | 2 | 2 | 2 | 316 | 35.4 | 5.63 |
| | OS = Glycine max PE = 2 SV = 1 - | | | | | | | | |
| | [C6TKR5_SOYBN] | | | | | | | | |
| C6TKT5 | Putative uncharacterized protein | 4.78 | 2 | 1 | 1 | 1 | 230 | 25.5 | 9.48 |
| | OS = Glycine max PE = 2 SV = 1 - | | | | | | | | |
| | [C6TKT5_SOYBN] | | | | | | | | |
| C6TKW9 | Putative uncharacterized protein | 30.13 | 2 | 8 | 12 | 14 | 458 | 49.3 | 7.49 |
| | OS = Glycine max PE = 2 SV = 1 - | | | | | | | | |
| | [C6TKW9_SOYBN] | | | | | | | | |
| C6TLD2 | Putative uncharacterized protein | 9.56 | 3 | 2 | 2 | 2 | 293 | 31.4 | 9.63 |
| | OS = Glycine max PE = 2 SV = 1 - | | | | | | | | |
| | [C6TLD2_SOYBN] | | | | | | | | |
| C6TLM5 | Putative uncharacterized protein | 3.95 | 3 | 1 | 1 | 1 | 253 | 27.7 | 8.46 |
| | OS = Glycine max PE = 2 SV = 1 - | | | | | | | | |
| | [C6TLM5_SOYBN] | | | | | | | | |
| C6TLN8 | Putative uncharacterized protein | 8.98 | 2 | 2 | 2 | 2 | 256 | 26.8 | 6.68 |
| | OS = Glycine max PE = 2 SV = 1 - | | | | | | | | |
| | [C6TLN8_SOYBN] | | | | | | | | |
| C6TM22 | Putative uncharacterized protein | 4.26 | 5 | 1 | 1 | 1 | 235 | 26.8 | 8.34 |
| | OS = Glycine max PE = 2 SV = 1 - | | | | | | | | |
| | [C6TM22_SOYBN] | | | | | | | | |
| C6TM44 | Putative uncharacterized protein | 5.07 | 2 | 1 | 1 | 1 | 276 | 31.5 | 5.27 |
| | OS = Glycine max PE = 2 SV = 1 - | | | | | | | | |
| | [C6TM44_SOYBN] | | | | | | | | |
| C6TMH2 | Putative uncharacterized protein | 7.51 | 3 | 1 | 1 | 1 | 213 | 22.8 | 6.02 |
| | OS = Glycine max PE = 2 SV = 1 - | | | | | | | | |
| | [C6TMH2_SOYBN] | | | | | | | | |
| C6TMK3 | Putative uncharacterized protein | 42.86 | 4 | 2 | 8 | 9 | 245 | 27.6 | 7.80 |
| | OS = Glycine max PE = 2 SV = 1 - | | | | | | | | |
| | [C6TMK3_SOYBN] | | | | | | | | |
| C6TML0 | Putative uncharacterized protein | 4.73 | 2 | 1 | 1 | 1 | 296 | 33.1 | 6.65 |
| | OS = Glycine max PE = 2 SV = 1 - | | | | | | | | |
| | [C6TML0_SOYBN] | | | | | | | | |
| C6TN97 | Putative uncharacterized protein | 13.27 | 6 | 3 | 3 | 3 | 422 | 46.8 | 6.11 |
| | OS = Glycine max PE = 2 SV = 1 - | | | | | | | | |
| | [C6TN97_SOYBN] | | | | | | | | |
| C6TNB6 | Putative uncharacterized protein | 5.54 | 4 | 1 | 1 | 1 | 397 | 43.1 | 6.74 |
| | OS = Glycine max PE = 2 SV = 1 - | | | | | | | | |
| | [C6TNB6_SOYBN] | | | | | | | | |
| C6TNP0 | Putative uncharacterized protein | 14.03 | 37 | 8 | 8 | 8 | 506 | 57.8 | 9.19 |
| | OS = Glycine max PE = 2 SV = 1 - | | | | | | | | |
| | [C6TNP0_SOYBN] | | | | | | | | |
| C6TNU2 | Putative uncharacterized protein | 24.64 | 5 | 1 | 9 | 14 | 418 | 47.5 | 6.02 |
| | OS = Glycine max PE = 2 SV = 1 - | | | | | | | | |
| | [C6TNU2_SOYBN] | | | | | | | | |
| C6TP03 | Putative uncharacterized protein | 4.85 | 2 | 1 | 1 | 1 | 268 | 30.2 | 6.42 |
| | OS = Glycine max PE = 2 SV = 1 - | | | | | | | | |
| | [C6TP03_SOYBN] | | | | | | | | |
| I1KMC2 | Pyruvate kinase OS = Glycine | 3.19 | 2 | 1 | 2 | 2 | 501 | 54.3 | 7.59 |
| | max PE = 3 SV = 1 - | | | | | | | | |
| | [I1KMC2_SOYBN] | | | | | | | | |
| I1L964 | Pyruvate kinase OS = Glycine | 17.08 | 7 | 2 | 7 | 7 | 527 | 57.6 | 7.50 |
| | max PE = 3 SV = 1 - | | | | | | | | |
| | [I1L964_SOYBN] | | | | | | | | |
| I1N5N0 | Pyruvate kinase OS = Glycine | 11.96 | 3 | 1 | 5 | 6 | 510 | 55.2 | 7.56 |
| | max PE = 3 SV = 1 - | | | | | | | | |
| | [I1N5N0_SOYBN] | | | | | | | | |
| I1NHP8 | Pyruvate kinase OS = Glycine | 11.33 | 3 | 1 | 3 | 3 | 415 | 45.8 | 5.68 |
| | max PE = 3 SV = 1 - | | | | | | | | |
| | [I1NHP8_SOYBN] | | | | | | | | |
| K7MZ60 | Pyruvate kinase OS = Glycine | 16.28 | 7 | 1 | 6 | 6 | 479 | 52.6 | 6.99 |
| | max PE = 3 SV = 1 - | | | | | | | | |
| | [K7MZ60_SOYBN] | | | | | | | | |
| Q42806 | Pymvate kinase, cytosolic | 12.13 | 2 | 1 | 5 | 6 | 511 | 55.3 | 7.56 |
| | isozyme OS = Glycine max PE = 2 | | | | | | | | |
| | SV = 1 - [KPYC_SOYBN] | | | | | | | | |
| C6TGW1 | Reticulon-like protein | 14.96 | 2 | 2 | 3 | 3 | 254 | 28.5 | 8.94 |
| | OS = Glycine max PE = 2 SV = 1 - | | | | | | | | |
| | [C6TGW1_SOYBN] | | | | | | | | |
| C6TH16 | Reticulon-like protein | 10.12 | 1 | 2 | 2 | 2 | 257 | 28.6 | 7.65 |
| | OS = Glycine max PE = 2 SV = 1 - | | | | | | | | |
| | [C6TH16_SOYBN] | | | | | | | | |
| C6TKL2 | Reticulon-like protein | 8.30 | 2 | 1 | 2 | 2 | 253 | 28.5 | 8.35 |
| | OS = Glycine max PE = 2 SV = 1 - | | | | | | | | |
| | [C6TKL2_SOYBN] | | | | | | | | |
| I1MKQ3 | Reticulon-like protein | 8.48 | 2 | 1 | 1 | 1 | 224 | 24.9 | 7.64 |
| | OS = Glycine max PE = 4 SV = 1 - | | | | | | | | |
| | [I1MKQ3_SOYBN] | | | | | | | | |
| C6SV78 | Ribosomal protein L15 | 22.06 | 4 | 1 | 5 | 5 | 204 | 24.1 | 11.63 |
| | OS = Glycine max PE = 2 SV = 1 - | | | | | | | | |
| | [C6SV78_SOYBN] | | | | | | | | |
| I1K0S0 | Ribosomal protein L15 | 22.06 | 5 | 1 | 5 | 6 | 204 | 24.2 | 11.59 |
| | OS = Glycine max PE = 3 SV = 1 - | | | | | | | | |
| | [I1K0S0_SOYBN] | | | | | | | | |
| C6SXM8 | Ribosomal protein L19 | 12.26 | 8 | 1 | 2 | 2 | 212 | 24.6 | 11.44 |
| | OS = Glycine max PE = 2 SV = 1 - | | | | | | | | |
| | [C6SXM8_SOYBN] | | | | | | | | |
| C6T1S2 | Ribosomal protein L19 | 12.26 | 8 | 1 | 2 | 2 | 212 | 24.7 | 11.46 |
| | OS = Glycine max PE = 2 SV = 1 - | | | | | | | | |
| | [C6T1S2_SOYBN] | | | | | | | | |
| C6T3U6 | Ribosomal protein L19 | 15.57 | 8 | 2 | 3 | 3 | 212 | 24.8 | 11.44 |
| | OS = Glycine max PE = 2 SV = 1 - | | | | | | | | |
| | [C6T3U6_SOYBN] | | | | | | | | |
| I1N6Q6 | Ribulose bisphosphate | 51.18 | 11 | 9 | 9 | 14 | 170 | 19.1 | 8.43 |
| | carboxylase small chain | | | | | | | | |
| | OS = Glycine max PE = 3 SV = 1 - | | | | | | | | |
| | [I1N6Q6_SOYBN] | | | | | | | | |
| C0J370 | Ribulose-1,5-bisphosphate | 23.47 | 22 | 11 | 11 | 19 | 473 | 52.3 | 6.54 |
| | carboxylase/oxygenase large | | | | | | | | |
| | subunit (Fragment) OS = Glycine | | | | | | | | |
| | max GN = rbcL PE = 3 SV = 1 - | | | | | | | | |
| | [C0J370_SOYBN] | | | | | | | | |
| I1JP95 | S-(hydroxymethyl)glutathione | 35.62 | 8 | 9 | 9 | 12 | 379 | 40.3 | 6.76 |
| | dehydrogenase OS = Glycine | | | | | | | | |
| | max PE = 3 SV = 1 - | | | | | | | | |
| | [I1JP95_SOYBN] | | | | | | | | |
| Q9S7N8 | Seed maturation protein PM21 | 53.68 | 2 | 3 | 3 | 6 | 95 | 10.1 | 5.01 |
| | OS = Glycine max GN = PM21 | | | | | | | | |
| | PE = 2 SV = 1 - | | | | | | | | |
| | [Q9S7N8_SOYBN] | | | | | | | | |
| Q9XER5 | Seed maturation protein PM22 | 41.45 | 1 | 5 | 5 | 5 | 152 | 16.7 | 5.36 |
| | OS = Glycine max GN = PM22 | | | | | | | | |
| | PE = 2 SV = 1 - | | | | | | | | |
| | [Q9XER5_SOYBN] | | | | | | | | |
| Q9SEK9 | Seed maturation protein PM25 | 72.33 | 3 | 7 | 9 | 30 | 253 | 25.7 | 5.10 |
| | OS = Glycine max GN = PM25 | | | | | | | | |
| | PE = 2 SV = 1 - | | | | | | | | |
| | [Q9SEK9_SOYBN] | | | | | | | | |
| Q9XES7 | Seed maturation protein PM27 | 19.76 | 3 | 4 | 5 | 8 | 339 | 38.0 | 9.38 |
| | OS = Glycine max GN = PM27 | | | | | | | | |
| | PE = 2 SV = 1 - | | | | | | | | |
| | [Q9XES7_SOYBN] | | | | | | | | |
| Q9XES8 | Seed maturation protein PM28 | 78.65 | 1 | 5 | 5 | 7 | 89 | 9.5 | 4.81 |
| | OS = Glycine max GN = PM28 | | | | | | | | |
| | PE = 2 SV = 1 - | | | | | | | | |
| | [Q9XES8_SOYBN] | | | | | | | | |
| Q9XET0 | Seed maturation protein PM30 | 49.29 | 1 | 6 | 6 | 9 | 140 | 15.1 | 8.90 |
| | OS = Glycine max GN = PM30 | | | | | | | | |
| | PE = 2 SV = 1 - | | | | | | | | |
| | [Q9XET0_SOYBN] | | | | | | | | |
| Q9XET1 | Seed maturation protein PM31 | 58.82 | 3 | 6 | 7 | 22 | 153 | 17.7 | 6.53 |
| | OS = Glycine max GN = PM31 | | | | | | | | |
| | PE = 2 SV = 1 - | | | | | | | | |
| | [Q9XET1_SOYBN] | | | | | | | | |
| Q9LLQ6 | Seed maturation protein PM34 | 39.93 | 2 | 2 | 9 | 26 | 293 | 31.7 | 7.08 |
| | OS = Glycine max GN = PM34 | | | | | | | | |
| | PE = 2 SV = 1 - | | | | | | | | |
| | [Q9LLQ6_SOYBN] | | | | | | | | |
| Q9SWB2 | Seed maturation protein PM41 | 32.05 | 3 | 3 | 3 | 3 | 78 | 8.2 | 4.97 |
| | OS = Glycine max GN = PM41 | | | | | | | | |
| | PE = 2 SV = 1 - | | | | | | | | |
| | [Q9SWB2_SOYBN] | | | | | | | | |
| K4FZF8 | Serine | 40.76 | 6 | 2 | 12 | 13 | 471 | 51.7 | 7.71 |
| | hydroxymethyltransferase | | | | | | | | |
| | OS = Glycine max GN = SHMT | | | | | | | | |
| | PE = 2 SV = 1 - | | | | | | | | |
| | [K4FZF8_SOYBN] | | | | | | | | |
| C6THM7 | Serine | 39.14 | 1 | 1 | 11 | 12 | 442 | 48.3 | 7.91 |
| | hydroxymethyltransferase | | | | | | | | |
| | OS = Glycine max PE = 2 SV = 1 - | | | | | | | | |
| | [C6THM7_SOYBN] | | | | | | | | |
| C6ZJY6 | Serine | 34.24 | 1 | 1 | 10 | 11 | 479 | 52.8 | 6.84 |
| | hydroxymethyltransferase | | | | | | | | |
| | OS = Glycine max PE = 2 SV = 1 - | | | | | | | | |
| | [C6ZJY6_SOYBN] | | | | | | | | |
| I1M1L2 | Serine | 2.91 | 2 | 1 | 1 | 1 | 378 | 41.5 | 7.08 |
| | hydroxymethyltransferase | | | | | | | | |
| | OS = Glycine max PE = 3 SV = 1 - | | | | | | | | |
| | [I1M1L2_SOYBN] | | | | | | | | |
| E9KNA6 | SGF14a OS = Glycine max PE = 2 | 36.19 | 16 | 3 | 7 | 7 | 257 | 29.0 | 4.75 |
| | SV = 1 - [E9KNA6_SOYBN] | | | | | | | | |
| E9KNA4 | SGF14h OS = Glycine max PE = 2 | 28.96 | 17 | 1 | 7 | 7 | 259 | 29.1 | 4.75 |
| | SV = 1 - [E9KNA4_SOYBN] | | | | | | | | |
| K7M8T7 | Signal peptidase I OS = Glycine | 4.58 | 6 | 1 | 1 | 1 | 153 | 17.2 | 7.12 |
| | max PE = 4 SV = 1 - | | | | | | | | |
| | [K7M8T7_SOYBN] | | | | | | | | |
| I1K3J0 | Signal recognition particle | 1.81 | 1 | 1 | 1 | 1 | 662 | 73.1 | 8.91 |
| | subunit SRP72 OS = Glycine | | | | | | | | |
| | max PE = 3 SV = 1 - | | | | | | | | |
| | [I1K3J0_SOYBN] | | | | | | | | |
| B2LUN7 | Starch synthase, | 5.59 | 3 | 3 | 4 | 4 | 608 | 66.8 | 7.96 |
| | chloroplastic/amyloplastic | | | | | | | | |
| | OS = Glycine max GN = GBSSIa | | | | | | | | |
| | PE = 2 SV = 1 - | | | | | | | | |
| | [B2LUN7_SOYBN] | | | | | | | | |
| I1MKC8 | Starch synthase, | 21.65 | 2 | 9 | 10 | 11 | 619 | 68.1 | 7.06 |
| | chloroplastic/amyloplastic | | | | | | | | |
| | OS = Glycine max PE = 3 SV = 1 - | | | | | | | | |
| | [I1MKC8_SOYBN] | | | | | | | | |
| I1LHR8 | Succinate dehydrogenase | 3.87 | 5 | 2 | 2 | 2 | 543 | 60.1 | 6.14 |
| | [ubiquinone] flavoprotein | | | | | | | | |
| | subunit, mitochondrial | | | | | | | | |
| | OS = Glycine max PE = 3 SV = 1 - | | | | | | | | |
| | [I1LHR8_SOYBN] | | | | | | | | |
| I1MLQ8 | Succinate dehydrogenase | 6.81 | 1 | 2 | 2 | 2 | 279 | 31.5 | 8.70 |
| | [ubiquinone] iron-sulfur | | | | | | | | |
| | subunit, mitochondrial | | | | | | | | |
| | OS = Glycine max PE = 3 SV = 1 - | | | | | | | | |
| | [I1MLQ8_SOYBN] | | | | | | | | |
| I1L8G3 | Succinyl-CoA ligase subunit | 1.66 | 2 | 1 | 1 | 1 | 422 | 45.3 | 6.23 |
| | beta OS = Glycine max PE = 3 | | | | | | | | |
| | SV = 1 - [I1L8G3_SOYBN] | | | | | | | | |
| K7KGC8 | Sucrose synthase OS = Glycine | 12.47 | 3 | 1 | 9 | 10 | 754 | 85.2 | 5.83 |
| | max PE = 3 SV = 1 - | | | | | | | | |
| | [K7KGC8_SOYBN] | | | | | | | | |
| K7MBG3 | Sucrose synthase OS = Glycine | 2.37 | 2 | 1 | 2 | 3 | 802 | 91.5 | 6.24 |
| | max PE = 3 SV = 1 - | | | | | | | | |
| | [K7MBG3_SOYBN] | | | | | | | | |
| K7MZJ1 | Sucrose synthase OS = Glycine | 16.18 | 4 | 3 | 11 | 12 | 754 | 85.1 | 6.09 |
| | max PE = 3 SV = 1 - | | | | | | | | |
| | [K7MZJ1_SOYBN] | | | | | | | | |
| Q04672 | Sucrose-binding protein | 62.21 | 1 | 2 | 29 | 168 | 524 | 60.5 | 6.87 |
| | OS = Glycine max GN = SBP | | | | | | | | |
| | PE = 1 SV = 1 - [SBP_SOYBN] | | | | | | | | |
| C6TH99 | Sulfurtransferase OS = Glycine | 4.65 | 3 | 1 | 1 | 1 | 215 | 24.0 | 8.28 |
| | max PE = 2 SV = 1 - | | | | | | | | |
| | [C6TH99_SOYBN] | | | | | | | | |
| C6T5D0 | Superoxide dismutase | 22.50 | 4 | 1 | 4 | 4 | 240 | 26.6 | 8.66 |
| | (Fragment) OS = Glycine max | | | | | | | | |
| | PE = 2 SV = 1 - | | | | | | | | |
| | [C6T5D0_SOYBN] | | | | | | | | |
| Q7M1R5 | Superoxide dismutase [Cu - Zn] | 8.55 | 1 | 1 | 1 | 1 | 152 | 15.2 | 5.60 |
| | OS = Glycine max GN = SOD1 | | | | | | | | |
| | PE = 3 SV = 1 - [SODC_SOYBN] | | | | | | | | |
| C6SWE8 | Superoxide dismutase [Cu - Zn] | 8.55 | 2 | 1 | 1 | 2 | 152 | 15.2 | 6.24 |
| | OS = Glycine max PE = 2 SV = 1 - | | | | | | | | |
| | [C6SWE8_SOYBN] | | | | | | | | |
| I1LKZ4 | Superoxide dismutase [Cu - Zn] | 25.62 | 7 | 3 | 3 | 4 | 203 | 20.8 | 6.52 |
| | OS = Glycine max PE = 3 SV = 1 - | | | | | | | | |
| | [I1LKZ4_SOYBN] | | | | | | | | |
| A5JVZ7 | Superoxide dismutase | 22.41 | 2 | 1 | 4 | 4 | 241 | 26.7 | 8.65 |
| | OS = Glycine max PE = 2 SV = 1 - | | | | | | | | |
| | [A5JVZ7_SOYBN] | | | | | | | | |
| C6TLJ0 | Superoxide dismutase | 13.71 | 13 | 4 | 4 | 4 | 248 | 27.8 | 5.48 |
| | OS = Glycine max PE = 2 SV = 1 - | | | | | | | | |
| | [C6TLJ0_SOYBN] | | | | | | | | |
| I1K467 | T-complex protein 1 subunit | 2.39 | 2 | 1 | 1 | 1 | 545 | 59.0 | 6.51 |
| | alpha OS = Glycine max PE = 3 | | | | | | | | |
| | SV = 1 - [I1K467_SOYBN] | | | | | | | | |
| I1KKD9 | T-complex protein 1 subunit | 3.72 | 9 | 2 | 2 | 2 | 511 | 55.1 | 7.53 |
| | delta OS = Glycine max PE = 3 | | | | | | | | |
| | SV = 2 - [I1KKD9_SOYBN] | | | | | | | | |
| I1L3P8 | T-complex protein 1 subunit | 12.25 | 5 | 5 | 5 | 5 | 400 | 44.0 | 6.57 |
| | gamma OS = Glycine max PE = 3 | | | | | | | | |
| | SV = 1 - [I1L3P8_SOYBN] | | | | | | | | |
| Q6PV94 | Thioredoxin OS = Glycine max | 5.93 | 2 | 1 | 1 | 1 | 135 | 14.8 | 6.54 |
| | PE = 2 SV = 1 - | | | | | | | | |
| | [Q6PV94_SOYBN] | | | | | | | | |
| Q2Q021 | Threonine synthase OS = Glycine | 2.14 | 3 | 1 | 1 | 1 | 515 | 56.3 | 7.14 |
| | max PE = 4 SV = 1 - | | | | | | | | |
| | [Q2Q021_SOYBN] | | | | | | | | |
| I1L1H7 | Translation | 4.76 | 4 | 1 | 1 | 1 | 189 | 20.7 | 7.64 |
| | machinery-associated protein 22 | | | | | | | | |
| | OS = Glycine max PE = 3 SV = 1 - | | | | | | | | |
| | [I1L1H7_SOYBN] | | | | | | | | |
| C6SZZ1 | Translocon-associated protein | 26.29 | 6 | 3 | 3 | 4 | 194 | 20.9 | 9.36 |
| | subunit beta OS = Glycine max | | | | | | | | |
| | PE = 2 SV = 1 - | | | | | | | | |
| | [C6SZZ1_SOYBN] | | | | | | | | |
| C6T6J1 | Triosephosphate isomerase | 45.56 | 4 | 1 | 6 | 12 | 180 | 19.5 | 6.55 |
| | (Fragment) OS = Glycine max | | | | | | | | |
| | PE = 2 SV = 1 - | | | | | | | | |
| | [C6T6J1_SOYBN] | | | | | | | | |
| Q38IW8 | Triosephosphate isomerase | 62.06 | 6 | 6 | 11 | 21 | 253 | 27.2 | 6.23 |
| | OS = Glycine max PE = 2 SV = 1 - | | | | | | | | |
| | [Q38IW8_SOYBN] | | | | | | | | |
| I1NAD0 | Triosephosphate isomerase | 10.78 | 5 | 2 | 3 | 4 | 306 | 33.0 | 7.68 |
| | OS = Glycine max PE = 3 SV = 1 - | | | | | | | | |
| | [I1NAD0_SOYBN] | | | | | | | | |
| C6K8D0 | Trypsin inhibitor 26 kDa | 39.92 | 2 | 7 | 7 | 10 | 238 | 25.9 | 8.32 |
| | isoform OS = Glycine max PE = 2 | | | | | | | | |
| | SV = 1 - [C6K8D0_SOYBN] | | | | | | | | |
| P01070 | Trypsin inhibitor A OS = Glycine | 52.31 | 9 | 7 | 14 | 100 | 216 | 24.0 | 5.11 |
| | max GN = KTI3 PE = 1 SV = 2 - | | | | | | | | |
| | [ITRA_SOYBN] | | | | | | | | |
| P01071 | Trypsin inhibitor B OS = Glycine | 55.25 | 3 | 2 | 9 | 61 | 181 | 20.0 | 4.82 |
| | max PE = 1 SV = 1 - | | | | | | | | |
| | [ITRB_SOYBN] | | | | | | | | |
| Q9LLX2 | Trypsin inhibitor OS = Glycine | 17.86 | 3 | 2 | 2 | 4 | 168 | 18.0 | 6.54 |
| | max PE = 2 SV = 1 - | | | | | | | | |
| | [Q9LLX2_SOYBN] | | | | | | | | |
| Q2TFP1 | Tubulin B4 OS = Glycine max | 27.62 | 9 | 1 | 10 | 13 | 449 | 50.4 | 4.83 |
| | PE = 2 SV = 1 - | | | | | | | | |
| | [Q2TFP1_SOYBN] | | | | | | | | |
| C6T574 | Ubiquitin carboxyl-terminal | 6.78 | 4 | 1 | 1 | 1 | 236 | 26.0 | 5.00 |
| | hydrolase (Fragment) | | | | | | | | |
| | OS = Glycine max PE = 2 SV = 1 - | | | | | | | | |
| | [C6T574_SOYBN] | | | | | | | | |
| I1JIM7 | Ubiquitin carboxyl-terminal | 2.24 | 6 | 2 | 2 | 2 | 1118 | 131.1 | 5.72 |
| | hydrolase OS = Glycine max | | | | | | | | |
| | PE = 3 SV = 1 - | | | | | | | | |
| | [I1JIM7_SOYBN] | | | | | | | | |
| I1KGF2 | Ubiquitin carboxyl-terminal | 2.50 | 2 | 1 | 1 | 1 | 480 | 54.2 | 6.14 |
| | hydrolase OS = Glycine max | | | | | | | | |
| | PE = 3 SV = 1 - | | | | | | | | |
| | [I1KGF2_SOYBN] | | | | | | | | |
| C6T8V3 | UDP-glucose 6-dehydrogenase | 8.12 | 10 | 3 | 3 | 3 | 468 | 51.6 | 6.79 |
| | OS = Glycine max PE = 2 SV = 1 - | | | | | | | | |
| | [C6T8V3_SOYBN] | | | | | | | | |
| C6T4P7 | Uncharacterized protein | 34.14 | 13 | 1 | 7 | 8 | 249 | 28.3 | 10.71 |
| | (Fragment) OS = Glycine max | | | | | | | | |
| | PE = 2 SV = 1 - | | | | | | | | |
| | [C6T4P7_SOYBN] | | | | | | | | |
| I1K0A0 | Uncharacterized protein | 7.42 | 3 | 2 | 3 | 3 | 418 | 46.1 | 5.94 |
| | (Fragment) OS = Glycine max | | | | | | | | |
| | PE = 3 SV = 1 - | | | | | | | | |
| | [I1K0A0_SOYBN] | | | | | | | | |
| K7KQ85 | Uncharacterized protein | 10.29 | 4 | 1 | 1 | 1 | 68 | 7.9 | 9.94 |
| | (Fragment) OS = Glycine max | | | | | | | | |
| | PE = 3 SV = 1 - | | | | | | | | |
| | [K7KQ85_SOYBN] | | | | | | | | |
| K7KRC0 | Uncharacterized protein | 10.67 | 10 | 4 | 4 | 4 | 431 | 48.2 | 5.63 |
| | (Fragment) OS = Glycine max | | | | | | | | |
| | PE = 3 SV = 1 - | | | | | | | | |
| | [K7KRC0_SOYBN] | | | | | | | | |
| K7LMI8 | Uncharacterized protein | 13.15 | 1 | 1 | 5 | 9 | 365 | 41.6 | 6.54 |
| | (Fragment) OS = Glycine max | | | | | | | | |
| | PE = 3 SV = 1 - | | | | | | | | |
| | [K7LMI8_SOYBN] | | | | | | | | |
| K7M625 | Uncharacterized protein | 2.91 | 6 | 1 | 1 | 1 | 412 | 47.2 | 6.64 |
| | (Fragment) OS = Glycine max | | | | | | | | |
| | PE = 3 SV = 1 - | | | | | | | | |
| | [K7M625_SOYBN] | | | | | | | | |
| K7N4A7 | Uncharacterized protein | 17.25 | 4 | 4 | 4 | 4 | 255 | 27.6 | 6.05 |
| | (Fragment) OS = Glycine max | | | | | | | | |
| | PE = 3 SV = 1 - | | | | | | | | |
| | [K7N4A7_SOYBN] | | | | | | | | |
| K7KFY0 | Uncharacterized protein | 12.93 | 3 | 2 | 2 | 2 | 147 | 15.7 | 5.55 |
| | (Fragment) OS = Glycine max | | | | | | | | |
| | PE = 4 SV = 1 - | | | | | | | | |
| | [K7KFY0_SOYBN] | | | | | | | | |
| K7KIL0 | Uncharacterized protein | 32.27 | 6 | 6 | 6 | 7 | 220 | 25.0 | 6.86 |
| | (Fragment) OS = Glycine max | | | | | | | | |
| | PE = 4 SV = 1 - | | | | | | | | |
| | [K7KIL0_SOYBN] | | | | | | | | |
| K7LHL1 | Uncharacterized protein | 17.53 | 5 | 1 | 1 | 1 | 97 | 11.0 | 9.19 |
| | (Fragment) OS = Glycine max | | | | | | | | |
| | PE = 4 SV = 1 - | | | | | | | | |
| | [K7LHL1_SOYBN] | | | | | | | | |
| K7LKW3 | Uncharacterized protein | 31.03 | 2 | 1 | 5 | 6 | 145 | 16.8 | 10.58 |
| | (Fragment) OS = Glycine max | | | | | | | | |
| | PE = 4 SV = 1 - | | | | | | | | |
| | [K7LKW3_SOYBN] | | | | | | | | |
| K7LR05 | Uncharacterized protein | 5.85 | 4 | 1 | 1 | 1 | 188 | 20.1 | 8.98 |
| | (Fragment) OS = Glycine max | | | | | | | | |
| | PE = 4 SV = 1 - | | | | | | | | |
| | [K7LR05_SOYBN] | | | | | | | | |
| K7MCB0 | Uncharacterized protein | 6.63 | 3 | 1 | 1 | 1 | 181 | 19.9 | 7.44 |
| | (Fragment) OS = Glycine max | | | | | | | | |
| | PE = 4 SV = 1 - | | | | | | | | |
| | [K7MCB0_SOYBN] | | | | | | | | |
| K7MND3 | Uncharacterized protein | 27.18 | 1 | 2 | 2 | 4 | 103 | 11.4 | 9.17 |
| | (Fragment) OS = Glycine max | | | | | | | | |
| | PE = 4 SV = 1 - | | | | | | | | |
| | [K7MND3_SOYBN] | | | | | | | | |
| C6SV94 | Uncharacterized protein | 53.33 | 3 | 6 | 6 | 11 | 150 | 16.3 | 10.87 |
| | OS = Glycine max PE = 2 SV = 1 - | | | | | | | | |
| | [C6SV94_SOYBN] | | | | | | | | |
| C6SVB6 | Uncharacterized protein | 12.00 | 2 | 2 | 2 | 2 | 175 | 19.9 | 6.29 |
| | OS = Glycine max PE = 2 SV = 1 - | | | | | | | | |
| | [C6SVB6_SOYBN] | | | | | | | | |
| C6SVC0 | Uncharacterized protein | 4.76 | 3 | 1 | 1 | 1 | 210 | 23.9 | 7.09 |
| | OS = Glycine max PE = 2 SV = 1 - | | | | | | | | |
| | [C6SVC0_SOYBN] | | | | | | | | |
| C6SVD6 | Uncharacterized protein | 16.46 | 5 | 1 | 1 | 1 | 79 | 8.8 | 8.76 |
| | OS = Glycine max PE = 2 SV = 1 - | | | | | | | | |
| | [C6SVD6_SOYBN] | | | | | | | | |
| C6SVF6 | Uncharacterized protein | 45.79 | 1 | 3 | 3 | 3 | 107 | 11.3 | 6.52 |
| | OS = Glycine max PE = 2 SV = 1 - | | | | | | | | |
| | [C6SVF6_SOYBN] | | | | | | | | |
| C6SVH4 | Uncharacterized protein | 18.49 | 1 | 1 | 2 | 2 | 146 | 16.0 | 6.80 |
| | OS = Glycine max PE = 2 SV = 1 - | | | | | | | | |
| | [C6SVH4_SOYBN] | | | | | | | | |
| C6SVI7 | Uncharacterized protein | 6.38 | 3 | 1 | 1 | 1 | 188 | 21.0 | 6.02 |
| | OS = Glycine max PE = 2 SV = 1 - | | | | | | | | |
| | [C6SVI7_SOYBN] | | | | | | | | |
| C6SVK2 | Uncharacterized protein | 31.93 | 5 | 2 | 3 | 3 | 119 | 13.5 | 9.45 |
| | OS = Glycine max PE = 2 SV = 1 - | | | | | | | | |
| | [C6SVK2_SOYBN] | | | | | | | | |
| C6SVM8 | Uncharacterized protein | 6.52 | 2 | 1 | 1 | 1 | 138 | 15.1 | 7.31 |
| | OS = Glycine max PE = 2 SV = 1 - | | | | | | | | |
| | [C6SVM8_SOYBN] | | | | | | | | |
| C6SVR2 | Uncharacterized protein | 20.00 | 2 | 2 | 3 | 4 | 120 | 13.9 | 9.82 |
| | OS = Glycine max PE = 2 SV = 1 - | | | | | | | | |
| | [C6SVR2_SOYBN] | | | | | | | | |
| C6SVT0 | Uncharacterized protein | 36.42 | 6 | 5 | 5 | 5 | 162 | 18.0 | 7.15 |
| | OS = Glycine max PE = 2 SV = 1 - | | | | | | | | |
| | [C6SVT0_SOYBN] | | | | | | | | |
| C6SVX5 | Uncharacterized protein | 46.85 | 8 | 10 | 10 | 16 | 222 | 25.1 | 10.35 |
| | OS = Glycine max PE = 2 SV = 1 - | | | | | | | | |
| | [C6SVX5_SOYBN] | | | | | | | | |
| C6SVY3 | Uncharacterized protein | 13.64 | 8 | 1 | 1 | 2 | 110 | 12.9 | 9.79 |
| | OS = Glycine max PE = 2 SV = 1 - | | | | | | | | |
| | [C6SVY3_SOYBN] | | | | | | | | |
| C6SW24 | Uncharacterized protein | 29.41 | 2 | 5 | 5 | 6 | 204 | 22.6 | 6.29 |
| | OS = Glycine max PE = 2 SV = 1 - | | | | | | | | |
| | [C6SW24_SOYBN] | | | | | | | | |
| C6SW35 | Uncharacterized protein | 13.33 | 1 | 1 | 2 | 2 | 120 | 13.8 | 9.86 |
| | OS = Glycine max PE = 2 SV = 1 - | | | | | | | | |
| | [C6SW35_SOYBN] | | | | | | | | |
| C6SW56 | Uncharacterized protein | 25.20 | 3 | 1 | 4 | 7 | 123 | 14.3 | 10.81 |
| | OS = Glycine max PE = 2 SV = 1 - | | | | | | | | |
| | [C6SW56_SOYBN] | | | | | | | | |
| C6SWA9 | Uncharacterized protein | 8.77 | 1 | 1 | 1 | 1 | 114 | 11.4 | 4.41 |
| | OS = Glycine max PE = 2 SV = 1 - | | | | | | | | |
| | [C6SWA9_SOYBN] | | | | | | | | |
| C6SWE0 | Uncharacterized protein | 56.17 | 4 | 6 | 6 | 13 | 162 | 17.4 | 5.59 |
| | OS = Glycine max PE = 2 SV = 1 - | | | | | | | | |
| | [C6SWE0_SOYBN] | | | | | | | | |
| C6SWG3 | Uncharacterized protein | 30.97 | 4 | 1 | 3 | 4 | 155 | 17.4 | 10.40 |
| | OS = Glycine max PE = 2 SV = 1 - | | | | | | | | |
| | [C6SWG3_SOYBN] | | | | | | | | |
| C6SWI3 | Uncharacterized protein | 21.01 | 3 | 2 | 2 | 2 | 138 | 14.3 | 9.70 |
| | OS = Glycine max PE = 2 SV = 1 - | | | | | | | | |
| | [C6SWI3_SOYBN] | | | | | | | | |
| C6SWL4 | Uncharacterized protein | 19.34 | 11 | 5 | 5 | 6 | 181 | 20.4 | 10.23 |
| | OS = Glycine max PE = 2 SV = 1 - | | | | | | | | |
| | [C6SWL4_SOYBN] | | | | | | | | |
| C6SWP9 | Uncharacterized protein | 5.60 | 3 | 1 | 1 | 1 | 232 | 24.8 | 9.85 |
| | OS = Glycine max PE = 2 SV = 1 - | | | | | | | | |
| | [C6SWP9_SOYBN] | | | | | | | | |
| C6SWV3 | Uncharacterized protein | 73.06 | 2 | 13 | 13 | 19 | 245 | 27.6 | 6.10 |
| | OS = Glycine max PE = 2 SV = 1 - | | | | | | | | |
| | [C6SWV3_SOYBN] | | | | | | | | |
| C6SWW4 | Uncharacterized protein | 48.28 | 4 | 7 | 8 | 73 | 203 | 22.4 | 5.01 |
| | OS = Glycine max PE = 2 SV = 1 - | | | | | | | | |
| | [C6SWW4_SOYBN] | | | | | | | | |
| C6SWX2 | Uncharacterized protein | 17.42 | 3 | 2 | 2 | 3 | 132 | 15.1 | 6.33 |
| | OS = Glycine max PE = 2 SV = 1 - | | | | | | | | |
| | [C6SWX2_SOYBN] | | | | | | | | |
| C6SX50 | Uncharacterized protein | 11.05 | 4 | 1 | 2 | 2 | 181 | 20.7 | 9.96 |
| | OS = Glycine max PE = 2 SV = 1 - | | | | | | | | |
| | [C6SX50_SOYBN] | | | | | | | | |
| C6SX81 | Uncharacterized protein | 6.25 | 2 | 1 | 1 | 1 | 144 | 15.5 | 4.77 |
| | OS = Glycine max PE = 2 SV = 1 - | | | | | | | | |
| | [C6SX81_SOYBN] | | | | | | | | |
| C6SX86 | Uncharacterized protein | 23.97 | 2 | 1 | 3 | 3 | 146 | 16.4 | 10.68 |
| | OS = Glycine max PE = 2 SV = 1 - | | | | | | | | |
| | [C6SX86_SOYBN] | | | | | | | | |
| C6SXB4 | Uncharacterized protein | 48.87 | 3 | 4 | 7 | 7 | 133 | 15.6 | 10.78 |
| | OS = Glycine max PE = 2 SV = 1 - | | | | | | | | |
| | [C6SXB4_SOYBN] | | | | | | | | |
| C6SXF0 | Uncharacterized protein | 9.18 | 2 | 1 | 1 | 1 | 207 | 22.6 | 10.23 |
| | OS = Glycine max PE = 2 SV = 1 - | | | | | | | | |
| | [C6SXF0_SOYBN] | | | | | | | | |
| C6SXG1 | Uncharacterized protein | 31.82 | 7 | 1 | 6 | 6 | 154 | 17.5 | 6.37 |
| | OS = Glycine max PE = 2 SV = 1 - | | | | | | | | |
| | [C6SXG1_SOYBN] | | | | | | | | |
| C6SXG4 | Uncharacterized protein | 20.44 | 2 | 1 | 2 | 2 | 137 | 15.2 | 7.20 |
| | OS = Glycine max PE = 2 SV = 1 - | | | | | | | | |
| | [C6SXG4_SOYBN] | | | | | | | | |
| C6SXG5 | Uncharacterized protein | 24.11 | 2 | 2 | 2 | 2 | 141 | 15.8 | 7.56 |
| | OS = Glycine max PE = 2 SV = 1 - | | | | | | | | |
| | [C6SXG5_SOYBN] | | | | | | | | |
| C6SXP0 | Uncharacterized protein | 20.44 | 2 | 1 | 2 | 2 | 137 | 15.2 | 8.69 |
| | OS = Glycine max PE = 2 SV = 1 - | | | | | | | | |
| | [C6SXP0_SOYBN] | | | | | | | | |
| C6SXR4 | Uncharacterized protein | 18.60 | 6 | 2 | 2 | 2 | 129 | 14.8 | 10.90 |
| | OS = Glycine max PE = 2 SV = 1 - | | | | | | | | |
| | [C6SXR4_SOYBN] | | | | | | | | |
| C6SY25 | Uncharacterized protein | 25.20 | 4 | 1 | 3 | 3 | 123 | 13.8 | 9.58 |
| | OS = Glycine max PE = 2 SV = 1 - | | | | | | | | |
| | [C6SY25_SOYBN] | | | | | | | | |
| C6SY43 | Uncharacterized protein | 13.04 | 2 | 1 | 2 | 3 | 184 | 21.3 | 6.23 |
| | OS = Glycine max PE = 2 SV = 1 - | | | | | | | | |
| | [C6SY43_SOYBN] | | | | | | | | |
| C6SY73 | Uncharacterized protein | 32.67 | 3 | 3 | 4 | 5 | 150 | 16.9 | 10.70 |
| | OS = Glycine max PE = 2 SV = 1 - | | | | | | | | |
| | [C6SY73_SOYBN] | | | | | | | | |
| C6SYC1 | Uncharacterized protein | 8.33 | 2 | 1 | 1 | 1 | 168 | 19.9 | 5.29 |
| | OS = Glycine max PE = 2 SV = 1 - | | | | | | | | |
| | [C6SYC1_SOYBN] | | | | | | | | |
| C6SYG6 | Uncharacterized protein | 5.00 | 1 | 1 | 1 | 1 | 200 | 21.8 | 4.84 |
| | OS = Glycine max PE = 2 SV = 1 - | | | | | | | | |
| | [C6SYG6_SOYBN] | | | | | | | | |
| C6SYJ4 | Uncharacterized protein | 15.65 | 2 | 1 | 2 | 2 | 147 | 16.5 | 10.71 |
| | OS = Glycine max PE = 2 SV = 1 - | | | | | | | | |
| | [C6SYJ4_SOYBN] | | | | | | | | |
| C6SYM8 | Uncharacterized protein | 22.50 | 1 | 3 | 3 | 4 | 200 | 21.9 | 5.12 |
| | OS = Glycine max PE = 2 SV = 1 - | | | | | | | | |
| | [C6SYM8_SOYBN] | | | | | | | | |
| C6SYZ8 | Uncharacterized protein | 24.76 | 4 | 2 | 5 | 5 | 206 | 23.6 | 10.32 |
| | OS = Glycine max PE = 2 SV = 1 - | | | | | | | | |
| | [C6SYZ8_SOYBN] | | | | | | | | |
| C6SZ13 | Uncharacterized protein | 24.49 | 1 | 3 | 3 | 9 | 147 | 15.8 | 9.36 |
| | OS = Glycine max PE = 2 SV = 1 - | | | | | | | | |
| | [C6SZ13_SOYBN] | | | | | | | | |
| C6SZ18 | Uncharacterized protein | 18.46 | 2 | 1 | 1 | 1 | 65 | 7.5 | 11.17 |
| | OS = Glycine max PE = 2 SV = 1 - | | | | | | | | |
| | [C6SZ18_SOYBN] | | | | | | | | |
| C6SZI5 | Uncharacterized protein | 11.56 | 3 | 2 | 2 | 2 | 173 | 19.0 | 8.62 |
| | OS = Glycine max PE = 2 SV = 1 - | | | | | | | | |
| | [C6SZI5_SOYBN] | | | | | | | | |
| C6SZI8 | Uncharacterized protein | 16.58 | 12 | 2 | 3 | 3 | 193 | 22.1 | 7.05 |
| | OS = Glycine max PE = 2 SV = 1 - | | | | | | | | |
| | [C6SZI8_SOYBN] | | | | | | | | |
| C6SZN6 | Uncharacterized protein | 20.12 | 2 | 1 | 4 | 4 | 164 | 17.9 | 5.91 |
| | OS = Glycine max PE = 2 SV = 1 - | | | | | | | | |
| | [C6SZN6_SOYBN] | | | | | | | | |
| C6SZQ3 | Uncharacterized protein | 37.09 | 10 | 5 | 5 | 6 | 151 | 17.2 | 10.21 |
| | OS = Glycine max PE = 2 SV = 1 - | | | | | | | | |
| | [C6SZQ3_SOYBN] | | | | | | | | |
| C6SZS1 | Uncharacterized protein | 26.22 | 3 | 5 | 5 | 6 | 164 | 18.7 | 10.51 |
| | OS = Glycine max PE = 2 SV = 1 - | | | | | | | | |
| | [C6SZS1_SOYBN] | | | | | | | | |
| C6SZX0 | Uncharacterized protein | 12.74 | 1 | 1 | 1 | 1 | 157 | 17.2 | 7.81 |
| | OS = Glycine max PE = 2 SV = 1 - | | | | | | | | |
| | [C6SZX0_SOYBN] | | | | | | | | |
| C6T034 | Uncharacterized protein | 54.26 | 1 | 2 | 8 | 14 | 223 | 24.3 | 4.68 |
| | OS = Glycine max PE = 2 SV = 1 - | | | | | | | | |
| | [C6T034_SOYBN] | | | | | | | | |
| C6T048 | Uncharacterized protein | 51.32 | 3 | 3 | 7 | 7 | 152 | 17.5 | 10.56 |
| | OS = Glycine max PE = 2 SV = 1 - | | | | | | | | |
| | [C6T048_SOYBN] | | | | | | | | |
| C6T073 | Uncharacterized protein | 31.98 | 3 | 5 | 5 | 5 | 197 | 21.2 | 8.63 |
| | OS = Glycine max PE = 2 SV = 1 - | | | | | | | | |
| | [C6T073_SOYBN] | | | | | | | | |
| C6T0B5 | Uncharacterized protein | 53.49 | 1 | 5 | 6 | 9 | 129 | 14.0 | 6.15 |
| | OS = Glycine max PE = 2 SV = 1 - | | | | | | | | |
| | [C6T0B5_SOYBN] | | | | | | | | |
| C6T1E4 | Uncharacterized protein | 8.77 | 1 | 1 | 1 | 1 | 114 | 11.4 | 4.37 |
| | OS = Glycine max PE = 2 SV = 1 - | | | | | | | | |
| | [C6T1E4_SOYBN] | | | | | | | | |
| C6T1J0 | Uncharacterized protein | 14.29 | 1 | 1 | 1 | 1 | 147 | 15.6 | 4.82 |
| | OS = Glycine max PE = 2 SV = 1 - | | | | | | | | |
| | [C6T1J0_SOYBN] | | | | | | | | |
| C6T1V2 | Uncharacterized protein | 29.30 | 1 | 2 | 4 | 8 | 157 | 17.7 | 6.81 |
| | OS = Glycine max PE = 2 SV = 1 - | | | | | | | | |
| | [C6T1V2_SOYBN] | | | | | | | | |
| C6T1W3 | Uncharacterized protein | 15.04 | 2 | 1 | 2 | 2 | 113 | 12.6 | 6.87 |
| | OS = Glycine max PE = 2 SV = 1 - | | | | | | | | |
| | [C6T1W3_SOYBN] | | | | | | | | |
| C6T2Y2 | Uncharacterized protein | 9.04 | 1 | 1 | 1 | 1 | 166 | 18.6 | 5.01 |
| | OS = Glycine max PE = 2 SV = 1 - | | | | | | | | |
| | [C6T2Y2_SOYBN] | | | | | | | | |
| C6T374 | Uncharacterized protein | 11.51 | 1 | 1 | 1 | 1 | 139 | 15.4 | 5.69 |
| | OS = Glycine max PE = 2 SV = 1 - | | | | | | | | |
| | [C6T374_SOYBN] | | | | | | | | |
| C6T3K0 | Uncharacterized protein | 9.31 | 1 | 1 | 1 | 1 | 204 | 22.5 | 4.96 |
| | OS = Glycine max PE = 2 SV = 1 - | | | | | | | | |
| | [C6T3K0_SOYBN] | | | | | | | | |
| C6T3K3 | Uncharacterized protein | 25.24 | 5 | 2 | 5 | 5 | 206 | 23.7 | 10.27 |
| | OS = Glycine max PE = 2 SV = 1 - | | | | | | | | |
| | [C6T3K3_SOYBN] | | | | | | | | |
| C6T4L4 | Uncharacterized protein | 11.11 | 2 | 1 | 1 | 1 | 90 | 9.9 | 8.29 |
| | OS = Glycine max PE = 2 SV = 1 - | | | | | | | | |
| | [C6T4L4_SOYBN] | | | | | | | | |
| C6T4P6 | Uncharacterized protein | 12.23 | 7 | 3 | 3 | 3 | 188 | 20.1 | 7.72 |
| | OS = Glycine max PE = 2 SV = 1 - | | | | | | | | |
| | [C6T4P6_SOYBN] | | | | | | | | |
| C6T4R9 | Uncharacterized protein | 45.16 | 20 | 1 | 6 | 14 | 155 | 17.7 | 9.74 |
| | OS = Glycine max PE = 2 SV = 1 - | | | | | | | | |
| | [C6T4R9_SOYBN] | | | | | | | | |
| C6T4Z6 | Uncharacterized protein | 35.66 | 1 | 1 | 5 | 5 | 143 | 15.9 | 10.18 |
| | OS = Glycine max PE = 2 SV = 1 - | | | | | | | | |
| | [C6T4Z6_SOYBN] | | | | | | | | |
| C6T588 | Uncharacterized protein | 43.04 | 6 | 1 | 5 | 8 | 158 | 16.8 | 4.88 |
| | OS = Glycine max PE = 2 SV = 1 - | | | | | | | | |
| | [C6T588_SOYBN] | | | | | | | | |
| C6T5E0 | Uncharacterized protein | 28.57 | 5 | 1 | 4 | 4 | 133 | 15.6 | 10.71 |
| | OS = Glycine max PE = 2 SV = 1 - | | | | | | | | |
| | [C6T5E0_SOYBN] | | | | | | | | |
| C6T670 | Uncharacterized protein | 14.86 | 3 | 1 | 2 | 3 | 148 | 16.6 | 10.71 |
| | OS = Glycine max PE = 2 SV = 1 - | | | | | | | | |
| | [C6T670_SOYBN] | | | | | | | | |
| C6T763 | Uncharacterized protein | 12.36 | 4 | 2 | 2 | 2 | 267 | 30.8 | 5.15 |
| | OS = Glycine max PE = 2 SV = 1 - | | | | | | | | |
| | [C6T763_SOYBN] | | | | | | | | |
| C6T7Y1 | Uncharacterized protein | 42.44 | 14 | 3 | 13 | 17 | 377 | 41.7 | 5.49 |
| | OS = Glycine max PE = 2 SV = 1 - | | | | | | | | |
| | [C6T7Y1_SOYBN] | | | | | | | | |
| C6T827 | Uncharacterized protein | 10.56 | 4 | 3 | 3 | 3 | 360 | 38.6 | 5.96 |
| | OS = Glycine max PE = 2 SV = 1 - | | | | | | | | |
| | [C6T827_SOYBN] | | | | | | | | |
| C6T868 | Uncharacterized protein | 9.36 | 2 | 2 | 2 | 2 | 267 | 28.7 | 5.27 |
| | OS = Glycine max PE = 2 SV = 1 - | | | | | | | | |
| | [C6T868_SOYBN] | | | | | | | | |
| C6T871 | Uncharacterized protein | 16.06 | 1 | 2 | 6 | 10 | 355 | 40.1 | 6.68 |
| | OS = Glycine max PE = 2 SV = 1 - | | | | | | | | |
| | [C6T871_SOYBN] | | | | | | | | |
| C6T8R7 | Uncharacterized protein | 17.74 | 7 | 2 | 4 | 6 | 265 | 28.6 | 5.66 |
| | OS = Glycine max PE = 2 SV = 1 - | | | | | | | | |
| | [C6T8R7_SOYBN] | | | | | | | | |
| C6T990 | Uncharacterized protein | 4.18 | 4 | 1 | 1 | 1 | 359 | 38.8 | 6.65 |
| | OS = Glycine max PE = 2 SV = 1 - | | | | | | | | |
| | [C6T990_SOYBN] | | | | | | | | |
| C6T9H6 | Uncharacterized protein | 2.31 | 2 | 1 | 1 | 1 | 389 | 42.3 | 7.56 |
| | OS = Glycine max PE = 2 SV = 1 - | | | | | | | | |
| | [C6T9H6_SOYBN] | | | | | | | | |
| C6TA47 | Uncharacterized protein | 10.71 | 5 | 1 | 1 | 1 | 112 | 12.5 | 5.25 |
| | OS = Glycine max PE = 2 SV = 1 - | | | | | | | | |
| | [C6TA47_SOYBN] | | | | | | | | |
| C6TA48 | Uncharacterized protein | 14.66 | 2 | 4 | 4 | 4 | 341 | 38.1 | 8.97 |
| | OS = Glycine max PE = 2 SV = 1 - | | | | | | | | |
| | [C6TA48_SOYBN] | | | | | | | | |
| C6TAU4 | Uncharacterized protein | 41.95 | 2 | 3 | 4 | 6 | 174 | 18.8 | 3.93 |
| | OS = Glycine max PE = 2 SV = 1 - | | | | | | | | |
| | [C6TAU4_SOYBN] | | | | | | | | |
| C6TAX7 | Uncharacterized protein | 46.73 | 1 | 7 | 7 | 7 | 214 | 24.2 | 5.77 |
| | OS = Glycine max PE = 2 SV = 1 - | | | | | | | | |
| | [C6TAX7_SOYBN] | | | | | | | | |
| C6TB56 | Uncharacterized protein | 6.18 | 3 | 1 | 1 | 1 | 356 | 38.6 | 7.20 |
| | OS = Glycine max PE = 2 SV = 1 - | | | | | | | | |
| | [C6TB56_SOYBN] | | | | | | | | |
| C6TBI4 | Uncharacterized protein | 24.11 | 5 | 3 | 3 | 3 | 112 | 12.8 | 10.61 |
| | OS = Glycine max PE = 2 SV = 1 - | | | | | | | | |
| | [C6TBI4_SOYBN] | | | | | | | | |
| C6TBU3 | Uncharacterized protein | 10.71 | 6 | 5 | 5 | 6 | 476 | 52.1 | 8.79 |
| | OS = Glycine max PE = 2 SV = 1 - | | | | | | | | |
| | [C6TBU3_SOYBN] | | | | | | | | |
| C6TBX7 | Uncharacterized protein | 10.59 | 1 | 1 | 1 | 1 | 85 | 9.7 | 9.79 |
| | OS = Glycine max PE = 2 SV = 1 - | | | | | | | | |
| | [C6TBX7_SOYBN] | | | | | | | | |
| C6TBY0 | Uncharacterized protein | 29.62 | 23 | 2 | 8 | 13 | 260 | 29.6 | 10.18 |
| | OS = Glycine max PE = 2 SV = 1 - | | | | | | | | |
| | [C6TBY0_SOYBN] | | | | | | | | |
| C6TCR3 | Uncharacterized protein | 8.27 | 1 | 1 | 1 | 1 | 133 | 14.6 | 7.11 |
| | OS = Glycine max PE = 2 SV = 1 - | | | | | | | | |
| | [C6TCR3_SOYBN] | | | | | | | | |
| C6TCX5 | Uncharacterized protein | 10.15 | 11 | 4 | 4 | 5 | 394 | 43.5 | 6.70 |
| | OS = Glycine max PE = 2 SV = 1 - | | | | | | | | |
| | [C6TCX5_SOYBN] | | | | | | | | |
| C6TD38 | Uncharacterized protein | 12.56 | 5 | 2 | 2 | 2 | 207 | 22.0 | 6.95 |
| | OS = Glycine max PE = 2 SV = 1 - | | | | | | | | |
| | [C6TD38_SOYBN] | | | | | | | | |
| C6TEP1 | Uncharacterized protein | 16.13 | 2 | 1 | 1 | 2 | 62 | 6.9 | 12.03 |
| | OS = Glycine max PE = 2 SV = 1 - | | | | | | | | |
| | [C6TEP1_SOYBN] | | | | | | | | |
| C6TEZ8 | Uncharacterized protein | 8.30 | 9 | 1 | 2 | 2 | 289 | 30.8 | 7.53 |
| | OS = Glycine max PE = 2 SV = 1 - | | | | | | | | |
| | [C6TEZ8_SOYBN] | | | | | | | | |
| C6TFE2 | Uncharacterized protein | 16.67 | 2 | 2 | 2 | 2 | 204 | 22.6 | 8.95 |
| | OS = Glycine max PE = 2 SV = 1 - | | | | | | | | |
| | [C6TFE2_SOYBN] | | | | | | | | |
| C6TFI8 | Uncharacterized protein | 20.55 | 4 | 2 | 4 | 5 | 146 | 16.7 | 11.02 |
| | OS = Glycine max PE = 2 SV = 1 - | | | | | | | | |
| | [C6TFI8_SOYBN] | | | | | | | | |
| C6TFP4 | Uncharacterized protein | 34.96 | 6 | 3 | 3 | 3 | 123 | 13.6 | 6.34 |
| | OS = Glycine max PE = 2 SV = 1 - | | | | | | | | |
| | [C6TFP4_SOYBN] | | | | | | | | |
| C6TFY7 | Uncharacterized protein | 61.86 | 6 | 6 | 6 | 6 | 97 | 10.6 | 8.40 |
| | OS = Glycine max PE = 2 SV = 1 - | | | | | | | | |
| | [C6TFY7_SOYBN] | | | | | | | | |
| C6TG81 | Uncharacterized protein | 4.79 | 1 | 1 | 1 | 1 | 313 | 34.6 | 5.12 |
| | OS = Glycine max PE = 2 SV = 1 - | | | | | | | | |
| | [C6TG81_SOYBN] | | | | | | | | |
| C6TGA6 | Uncharacterized protein | 27.27 | 3 | 2 | 5 | 7 | 319 | 34.2 | 5.11 |
| | OS = Glycine max PE = 2 SV = 1 - | | | | | | | | |
| | [C6TGA6_SOYBN] | | | | | | | | |
| C6TGW2 | Uncharacterized protein | 28.24 | 11 | 2 | 7 | 7 | 262 | 29.3 | 4.75 |
| | OS = Glycine max PE = 2 SV = 1 - | | | | | | | | |
| | [C6TGW2_SOYBN] | | | | | | | | |
| C6TGZ7 | Uncharacterized protein | 12.68 | 1 | 1 | 3 | 3 | 276 | 29.8 | 7.68 |
| | OS = Glycine max PE = 2 SV = 1 - | | | | | | | | |
| | [C6TGZ7_SOYBN] | | | | | | | | |
| C6THR8 | Uncharacterized protein | 14.20 | 1 | 1 | 4 | 5 | 331 | 35.6 | 8.76 |
| | OS = Glycine max PE = 2 SV = 1 - | | | | | | | | |
| | [C6THR8_SOYBN] | | | | | | | | |
| C6THW8 | Uncharacterized protein | 8.66 | 5 | 2 | 2 | 2 | 254 | 28.0 | 6.61 |
| | OS = Glycine max PE = 2 SV = 1 - | | | | | | | | |
| | [C6THW8_SOYBN] | | | | | | | | |
| C6TID4 | Uncharacterized protein | 12.00 | 2 | 4 | 4 | 4 | 325 | 34.5 | 6.24 |
| | OS = Glycine max PE = 2 SV = 1 - | | | | | | | | |
| | [C6TID4_SOYBN] | | | | | | | | |
| C6TIN6 | Uncharacterized protein | 15.05 | 12 | 4 | 4 | 5 | 392 | 41.2 | 4.84 |
| | OS = Glycine max PE = 2 SV = 1 - | | | | | | | | |
| | [C6TIN6_SOYBN] | | | | | | | | |
| C6TIQ5 | Uncharacterized protein | 27.70 | 11 | 9 | 9 | 9 | 343 | 37.9 | 6.32 |
| | OS = Glycine max PE = 2 SV = 1 - | | | | | | | | |
| | [C6TIQ5_SOYBN] | | | | | | | | |
| C6TIQ8 | Uncharacterized protein | 2.28 | 1 | 1 | 1 | 1 | 438 | 47.9 | 7.58 |
| | OS = Glycine max PE = 2 SV = 1 - | | | | | | | | |
| | [C6TIQ8_SOYBN] | | | | | | | | |
| C6TJD3 | Uncharacterized protein | 56.31 | 3 | 13 | 13 | 21 | 325 | 35.7 | 7.72 |
| | OS = Glycine max PE = 2 SV = 1 - | | | | | | | | |
| | [C6TJD3_SOYBN] | | | | | | | | |
| C6TJF3 | Uncharacterized protein | 3.87 | 2 | 1 | 1 | 1 | 310 | 33.8 | 5.08 |
| | OS = Glycine max PE = 2 SV = 1 - | | | | | | | | |
| | [C6TJF3_SOYBN] | | | | | | | | |
| C6TJG0 | Uncharacterized protein | 23.72 | 3 | 5 | 5 | 5 | 253 | 26.6 | 7.39 |
| | OS = Glycine max PE = 2 SV = 1 - | | | | | | | | |
| | [C6TJG0_SOYBN] | | | | | | | | |
| C6TKH0 | Uncharacterized protein | 45.58 | 1 | 10 | 10 | 28 | 294 | 31.6 | 6.84 |
| | OS = Glycine max PE = 2 SV = 1 - | | | | | | | | |
| | [C6TKH0_SOYBN] | | | | | | | | |
| C6TKK5 | Uncharacterized protein | 9.84 | 2 | 1 | 2 | 2 | 193 | 22.1 | 6.55 |
| | OS = Glycine max PE = 2 SV = 1 - | | | | | | | | |
| | [C6TKK5_SOYBN] | | | | | | | | |
| C6TKQ3 | Uncharacterized protein | 16.50 | 1 | 5 | 5 | 5 | 309 | 34.7 | 6.37 |
| | OS = Glycine max PE = 2 SV = 1 - | | | | | | | | |
| | [C6TKQ3_SOYBN] | | | | | | | | |
| C6TKV3 | Uncharacterized protein | 4.01 | 2 | 2 | 2 | 2 | 424 | 46.1 | 5.96 |
| | OS = Glycine max PE = 2 SV = 1 - | | | | | | | | |
| | [C6TKV3_SOYBN] | | | | | | | | |
| C6TLM4 | Uncharacterized protein | 30.80 | 10 | 3 | 5 | 7 | 263 | 27.9 | 5.43 |
| | OS = Glycine max PE = 2 SV = 1 - | | | | | | | | |
| | [C6TLM4_SOYBN] | | | | | | | | |
| C6TMA8 | Uncharacterized protein | 40.34 | 2 | 3 | 3 | 3 | 119 | 12.2 | 4.86 |
| | OS = Glycine max PE = 2 SV = 1 - | | | | | | | | |
| | [C6TMA8_SOYBN] | | | | | | | | |
| C6TMV9 | Uncharacterized protein | 29.59 | 3 | 6 | 6 | 7 | 294 | 31.4 | 8.22 |
| | OS = Glycine max PE = 2 SV = 1 - | | | | | | | | |
| | [C6TMV9_SOYBN] | | | | | | | | |
| C6TNR9 | Uncharacterized protein | 4.81 | 1 | 1 | 1 | 2 | 291 | 32.2 | 8.32 |
| | OS = Glycine max PE = 2 SV = 1 - | | | | | | | | |
| | [C6TNR9_SOYBN] | | | | | | | | |
| C6TNV4 | Uncharacterized protein | 6.79 | 2 | 2 | 2 | 2 | 324 | 33.8 | 8.76 |
| | OS = Glycine max PE = 2 SV = 1 - | | | | | | | | |
| | [C6TNV4_SOYBN] | | | | | | | | |
| I1J5B4 | Uncharacterized protein | 5.30 | 2 | 4 | 4 | 5 | 717 | 82.3 | 7.46 |
| | OS = Glycine max PE = 3 SV = 1 - | | | | | | | | |
| | [I1J5B4_SOYBN] | | | | | | | | |
| I1J752 | Uncharacterized protein | 25.39 | 5 | 2 | 9 | 13 | 449 | 50.6 | 4.87 |
| | OS = Glycine max PE = 3 SV = 1 - | | | | | | | | |
| | [I1J752_SOYBN] | | | | | | | | |
| I1J8Y0 | Uncharacterized protein | 11.26 | 5 | 3 | 5 | 5 | 657 | 73.0 | 5.73 |
| | OS = Glycine max PE = 3 SV = 1 - | | | | | | | | |
| | [I1J8Y0_SOYBN] | | | | | | | | |
| I1JAE4 | Uncharacterized protein | 2.48 | 8 | 1 | 1 | 1 | 322 | 35.1 | 7.09 |
| | OS = Glycine max PE = 3 SV = 1 - | | | | | | | | |
| | [I1JAE4_SOYBN] | | | | | | | | |
| I1JAU2 | Uncharacterized protein | 4.03 | 1 | 2 | 2 | 2 | 571 | 62.0 | 5.68 |
| | OS = Glycine max PE = 3 SV = 1 - | | | | | | | | |
| | [I1JAU2_SOYBN] | | | | | | | | |
| I1JB54 | Uncharacterized protein | 38.32 | 10 | 3 | 3 | 3 | 107 | 11.9 | 9.38 |
| | OS = Glycine max PE = 3 SV = 1 - | | | | | | | | |
| | [I1JB54_SOYBN] | | | | | | | | |
| I1JD52 | Uncharacterized protein | 12.48 | 7 | 5 | 5 | 6 | 545 | 58.6 | 5.40 |
| | OS = Glycine max PE = 3 SV = 1 - | | | | | | | | |
| | [I1JD52_SOYBN] | | | | | | | | |
| I1JDH6 | Uncharacterized protein | 30.55 | 3 | 1 | 7 | 9 | 275 | 30.2 | 10.36 |
| | OS = Glycine max PE = 3 SV = 1 - | | | | | | | | |
| | [I1JDH6_SOYBN] | | | | | | | | |
| I1JE09 | Uncharacterized protein | 54.49 | 1 | 1 | 7 | 7 | 156 | 17.7 | 10.20 |
| | OS = Glycine max PE = 3 SV = 1 - | | | | | | | | |
| | [I1JE09_SOYBN] | | | | | | | | |
| I1JFW7 | Uncharacterized protein | 35.38 | 9 | 6 | 6 | 7 | 195 | 21.8 | 9.29 |
| | OS = Glycine max PE = 3 SV = 1 - | | | | | | | | |
| | [I1JFW7_SOYBN] | | | | | | | | |
| I1JGR5 | Uncharacterized protein | 33.74 | 8 | 2 | 18 | 22 | 652 | 71.6 | 5.31 |
| | OS = Glycine max PE = 3 SV = 1 - | | | | | | | | |
| | [I1JGR5_SOYBN] | | | | | | | | |
| I1JIA6 | Uncharacterized protein | 17.39 | 2 | 1 | 1 | 1 | 69 | 7.7 | 9.72 |
| | OS = Glycine max PE = 3 SV = 1 - | | | | | | | | |
| | [I1JIA6_SOYBN] | | | | | | | | |
| I1JIP5 | Uncharacterized protein | 6.79 | 2 | 4 | 4 | 4 | 560 | 60.2 | 6.58 |
| | OS = Glycine max PE = 3 SV = 1 - | | | | | | | | |
| | [I1JIP5_SOYBN] | | | | | | | | |
| I1JJT3 | Uncharacterized protein | 4.25 | 4 | 1 | 1 | 1 | 212 | 24.4 | 6.47 |
| | OS = Glycine max PE = 3 SV = 1 - | | | | | | | | |
| | [I1JJT3_SOYBN] | | | | | | | | |
| I1JJT9 | Uncharacterized protein | 3.31 | 4 | 1 | 1 | 1 | 362 | 37.5 | 5.55 |
| | OS = Glycine max PE = 3 SV = 1 - | | | | | | | | |
| | [I1JJT9_SOYBN] | | | | | | | | |
| I1JML1 | Uncharacterized protein | 5.08 | 2 | 1 | 1 | 1 | 197 | 20.8 | 7.58 |
| | OS = Glycine max PE = 3 SV = 1 - | | | | | | | | |
| | [I1JML1_SOYBN] | | | | | | | | |
| I1JPC5 | Uncharacterized protein | 27.14 | 7 | 2 | 13 | 18 | 619 | 67.5 | 5.08 |
| | OS = Glycine max PE = 3 SV = 1 - | | | | | | | | |
| | [I1JPC5_SOYBN] | | | | | | | | |
| I1JPP3 | Uncharacterized protein | 25.99 | 7 | 2 | 18 | 19 | 808 | 89.6 | 5.26 |
| | OS = Glycine max PE = 3 SV = 1 - | | | | | | | | |
| | [I1JPP3_SOYBN] | | | | | | | | |
| I1JPS0 | Uncharacterized protein | 8.63 | 3 | 1 | 1 | 2 | 197 | 20.2 | 6.01 |
| | OS = Glycine max PE = 3 SV = 1 - | | | | | | | | |
| | [I1JPS0_SOYBN] | | | | | | | | |
| I1JPV8 | Uncharacterized protein | 2.91 | 1 | 1 | 1 | 1 | 516 | 59.1 | 8.24 |
| | OS = Glycine max PE = 3 SV = 1 - | | | | | | | | |
| | [I1JPV8_SOYBN] | | | | | | | | |
| I1JPW5 | Uncharacterized protein | 36.71 | 6 | 3 | 11 | 18 | 444 | 47.6 | 5.69 |
| | OS = Glycine max PE = 3 SV = 1 - | | | | | | | | |
| | [I1JPW5_SOYBN] | | | | | | | | |
| I1JPZ4 | Uncharacterized protein | 8.85 | 1 | 1 | 1 | 1 | 113 | 11.3 | 4.37 |
| | OS = Glycine max PE = 3 SV = 1 - | | | | | | | | |
| | [I1JPZ4_SOYBN] | | | | | | | | |
| I1JQX3 | Uncharacterized protein | 15.34 | 4 | 6 | 6 | 6 | 515 | 58.6 | 6.49 |
| | OS = Glycine max PE = 3 SV = 1 - | | | | | | | | |
| | [I1JQX3_SOYBN] | | | | | | | | |
| I1JRF2 | Uncharacterized protein | 12.75 | 3 | 5 | 5 | 6 | 557 | 62.7 | 7.09 |
| | OS = Glycine max PE = 3 SV = 1 - | | | | | | | | |
| | [I1JRF2_SOYBN] | | | | | | | | |
| I1JS37 | Uncharacterized protein | 43.29 | 3 | 2 | 3 | 4 | 164 | 17.4 | 8.43 |
| | OS = Glycine max PE = 3 SV = 1 - | | | | | | | | |
| | [I1JS37_SOYBN] | | | | | | | | |
| I1JS70 | Uncharacterized protein | 17.20 | 9 | 5 | 5 | 5 | 378 | 42.2 | 5.63 |
| | OS = Glycine max PE = 3 SV = 1 - | | | | | | | | |
| | [I1JS70_SOYBN] | | | | | | | | |
| I1JSC6 | Uncharacterized protein | 5.70 | 3 | 1 | 1 | 1 | 263 | 28.8 | 10.45 |
| | OS = Glycine max PE = 3 SV = 1 - | | | | | | | | |
| | [I1JSC6_SOYBN] | | | | | | | | |
| I1JTG3 | Uncharacterized protein | 7.46 | 3 | 3 | 3 | 3 | 389 | 42.7 | 6.57 |
| | OS = Glycine max PE = 3 SV = 1 - | | | | | | | | |
| | [I1JTG3_SOYBN] | | | | | | | | |
| I1JU00 | Uncharacterized protein | 15.19 | 1 | 1 | 2 | 2 | 158 | 17.9 | 7.18 |
| | OS = Glycine max PE = 3 SV = 1 - | | | | | | | | |
| | [I1JU00_SOYBN] | | | | | | | | |
| I1JXA0 | Uncharacterized protein | 33.29 | 3 | 2 | 23 | 27 | 814 | 90.8 | 5.24 |
| | OS = Glycine max PE = 3 SV = 1 - | | | | | | | | |
| | [I1JXA0_SOYBN] | | | | | | | | |
| I1JXQ3 | Uncharacterized protein | 9.15 | 4 | 3 | 3 | 3 | 306 | 32.4 | 9.99 |
| | OS = Glycine max PE = 3 SV = 1 - | | | | | | | | |
| | [I1JXQ3_SOYBN] | | | | | | | | |
| I1JXS7 | Uncharacterized protein | 28.67 | 1 | 4 | 14 | 17 | 579 | 66.1 | 4.89 |
| | OS = Glycine max PE = 3 SV = 1 - | | | | | | | | |
| | [I1JXS7_SOYBN] | | | | | | | | |
| I1JXY2 | Uncharacterized protein | 3.72 | 2 | 3 | 3 | 3 | 914 | 103.9 | 6.40 |
| | OS = Glycine max PE = 3 SV = 1 - | | | | | | | | |
| | [I1JXY2_SOYBN] | | | | | | | | |
| I1JYI8 | Uncharacterized protein | 20.00 | 3 | 2 | 2 | 3 | 110 | 11.1 | 4.32 |
| | OS = Glycine max PE = 3 SV = 1 - | | | | | | | | |
| | [I1JYI8_SOYBN] | | | | | | | | |
| I1JZP0 | Uncharacterized protein | 16.95 | 2 | 1 | 6 | 6 | 413 | 43.6 | 6.92 |
| | OS = Glycine max PE = 3 SV = 1 - | | | | | | | | |
| | [I1JZP0_SOYBN] | | | | | | | | |
| I1JZW5 | Uncharacterized protein | 9.02 | 4 | 3 | 3 | 4 | 488 | 52.8 | 5.40 |
| | OS = Glycine max PE = 3 SV = 1 - | | | | | | | | |
| | [I1JZW5_SOYBN] | | | | | | | | |
| I1K0H2 | Uncharacterized protein | 41.43 | 4 | 5 | 5 | 5 | 140 | 16.0 | 10.52 |
| | OS = Glycine max PE = 3 SV = 1 - | | | | | | | | |
| | [I1K0H2_SOYBN] | | | | | | | | |
| I1K2I1 | Uncharacterized protein | 24.00 | 5 | 4 | 8 | 12 | 450 | 49.6 | 5.10 |
| | OS = Glycine max PE = 3 SV = 1 - | | | | | | | | |
| | [I1K2I1_SOYBN] | | | | | | | | |
| I1K3X9 | Uncharacterized protein | 22.27 | 7 | 4 | 8 | 12 | 449 | 49.6 | 5.14 |
| | OS = Glycine max PE = 3 SV = 1 - | | | | | | | | |
| | [I1K3X9_SOYBN] | | | | | | | | |
| I1K3Y4 | Uncharacterized protein | 6.17 | 4 | 1 | 1 | 1 | 243 | 26.1 | 9.31 |
| | OS = Glycine max PE = 3 SV = 1 - | | | | | | | | |
| | [I1K3Y4_SOYBN] | | | | | | | | |
| I1K5K3 | Uncharacterized protein | 30.88 | 2 | 1 | 7 | 9 | 272 | 29.8 | 10.32 |
| | OS = Glycine max PE = 3 SV = 1 - | | | | | | | | |
| | [I1K5K3_SOYBN] | | | | | | | | |
| I1K622 | Uncharacterized protein | 1.59 | 2 | 1 | 1 | 1 | 567 | 63.3 | 6.23 |
| | OS = Glycine max PE = 3 SV = 1 - | | | | | | | | |
| | [I1K622_SOYBN] | | | | | | | | |
| I1K670 | Uncharacterized protein | 44.01 | 16 | 5 | 22 | 32 | 668 | 73.6 | 5.19 |
| | OS = Glycine max PE = 3 SV = 1 - | | | | | | | | |
| | [I1K670_SOYBN] | | | | | | | | |
| I1KAJ4 | Uncharacterized protein | 36.10 | 2 | 1 | 10 | 20 | 349 | 37.2 | 6.46 |
| | OS = Glycine max PE = 3 SV = 1 - | | | | | | | | |
| | [I1KAJ4_SOYBN] | | | | | | | | |
| I1KAJ5 | Uncharacterized protein | 38.58 | 2 | 0 | 10 | 20 | 337 | 36.9 | 6.15 |
| | OS = Glycine max PE = 3 SV = 1 - | | | | | | | | |
| | [I1KAJ5_SOYBN] | | | | | | | | |
| I1KAY1 | Uncharacterized protein | 14.29 | 2 | 1 | 1 | 1 | 98 | 11.1 | 5.02 |
| | OS = Glycine max PE = 3 SV = 1 - | | | | | | | | |
| | [I1KAY1_SOYBN] | | | | | | | | |
| I1KC56 | Uncharacterized protein | 5.34 | 2 | 2 | 2 | 2 | 487 | 53.3 | 6.80 |
| | OS = Glycine max PE = 3 SV = 1 - | | | | | | | | |
| | [I1KC56_SOYBN] | | | | | | | | |
| I1KCK6 | Uncharacterized protein | 4.38 | 4 | 1 | 1 | 1 | 457 | 49.7 | 5.57 |
| | OS = Glycine max PE = 3 SV = 1 - | | | | | | | | |
| | [I1KCK6_SOYBN] | | | | | | | | |
| I1KDL8 | Uncharacterized protein | 34.17 | 10 | 4 | 4 | 4 | 120 | 13.6 | 6.55 |
| | OS = Glycine max PE = 3 SV = 1 - | | | | | | | | |
| | [I1KDL8_SOYBN] | | | | | | | | |
| I1KEN4 | Uncharacterized protein | 6.21 | 4 | 2 | 5 | 5 | 966 | 110.2 | 5.74 |
| | OS = Glycine max PE = 3 SV = 1 - | | | | | | | | |
| | [I1KEN4_SOYBN] | | | | | | | | |
| I1KGH6 | Uncharacterized protein | 2.77 | 2 | 1 | 1 | 1 | 505 | 54.4 | 6.27 |
| | OS = Glycine max PE = 3 SV = 1 - | | | | | | | | |
| | [I1KGH6_SOYBN] | | | | | | | | |
| I1KGP0 | Uncharacterized protein | 43.21 | 8 | 2 | 11 | 12 | 243 | 28.4 | 9.99 |
| | OS = Glycine max PE = 3 SV = 1 - | | | | | | | | |
| | [I1KGP0_SOYBN] | | | | | | | | |
| I1KIE4 | Uncharacterized protein | 22.48 | 2 | 5 | 5 | 5 | 298 | 32.2 | 6.80 |
| | OS = Glycine max PE = 3 SV = 1 - | | | | | | | | |
| | [I1KIE4_SOYBN] | | | | | | | | |
| I1KIP9 | Uncharacterized protein | 1.80 | 2 | 1 | 1 | 1 | 724 | 78.1 | 9.23 |
| | OS = Glycine max PE = 3 SV = 1 - | | | | | | | | |
| | [I1KIP9_SOYBN] | | | | | | | | |
| I1KKN7 | Uncharacterized protein | 23.34 | 2 | 11 | 11 | 11 | 617 | 67.4 | 7.27 |
| | OS = Glycine max PE = 3 SV = 1 - | | | | | | | | |
| | [I1KKN7_SOYBN] | | | | | | | | |
| I1KL89 | Uncharacterized protein | 29.04 | 2 | 10 | 10 | 13 | 458 | 49.8 | 6.65 |
| | OS = Glycine max PE = 3 SV = 1 - | | | | | | | | |
| | [I1KL89_SOYBN] | | | | | | | | |
| I1KMP3 | Uncharacterized protein | 2.41 | 2 | 1 | 1 | 1 | 497 | 54.2 | 6.48 |
| | OS = Glycine max PE = 3 SV = 1 - | | | | | | | | |
| | [I1KMP3_SOYBN] | | | | | | | | |
| I1KNG1 | Uncharacterized protein | 7.03 | 4 | 2 | 4 | 4 | 512 | 55.4 | 6.90 |
| | OS = Glycine max PE = 3 SV = 1 - | | | | | | | | |
| | [I1KNG1_SOYBN] | | | | | | | | |
| I1KNH5 | Uncharacterized protein | 10.44 | 8 | 3 | 5 | 6 | 546 | 59.9 | 6.98 |
| | OS = Glycine max PE = 3 SV = 1 - | | | | | | | | |
| | [I1KNH5_SOYBN] | | | | | | | | |
| I1KNL8 | Uncharacterized protein | 4.35 | 4 | 1 | 1 | 1 | 345 | 35.9 | 6.25 |
| | OS = Glycine max PE = 3 SV = 1 - | | | | | | | | |
| | [I1KNL8_SOYBN] | | | | | | | | |
| I1KP72 | Uncharacterized protein | 20.12 | 9 | 5 | 5 | 5 | 323 | 36.0 | 8.12 |
| | OS = Glycine max PE = 3 SV = 1 - | | | | | | | | |
| | [I1KP72_SOYBN] | | | | | | | | |
| I1KPN3 | Uncharacterized protein | 35.08 | 8 | 1 | 18 | 28 | 667 | 73.4 | 5.19 |
| | OS = Glycine max PE = 3 SV = 1 - | | | | | | | | |
| | [I1KPN3_SOYBN] | | | | | | | | |
| I1KPV1 | Uncharacterized protein | 2.95 | 2 | 1 | 1 | 1 | 509 | 54.8 | 5.77 |
| | OS = Glycine max PE = 3 SV = 1 - | | | | | | | | |
| | [I1KPV1_SOYBN] | | | | | | | | |
| I1KQ93 | Uncharacterized protein | 20.10 | 4 | 8 | 8 | 9 | 582 | 63.4 | 5.53 |
| | OS = Glycine max PE = 3 SV = 1 - | | | | | | | | |
| | [I1KQ93_SOYBN] | | | | | | | | |
| I1KQE3 | Uncharacterized protein | 54.19 | 2 | 3 | 7 | 8 | 155 | 17.4 | 10.20 |
| | OS = Glycine max PE = 3 SV = 1 - | | | | | | | | |
| | [I1KQE3_SOYBN] | | | | | | | | |
| I1KSB7 | Uncharacterized protein | 2.82 | 1 | 1 | 1 | 1 | 496 | 53.0 | 5.16 |
| | OS = Glycine max PE = 3 SV = 1 - | | | | | | | | |
| | [I1KSB7_SOYBN] | | | | | | | | |
| I1KSE0 | Uncharacterized protein | 15.10 | 11 | 1 | 3 | 3 | 245 | 27.9 | 5.08 |
| | OS = Glycine max PE = 3 SV = 1 - | | | | | | | | |
| | [I1KSE0_SOYBN] | | | | | | | | |
| I1KT57 | Uncharacterized protein | 7.03 | 5 | 1 | 1 | 1 | 327 | 35.4 | 8.31 |
| | OS = Glycine max PE = 3 SV = 1 - | | | | | | | | |
| | [I1KT57_SOYBN] | | | | | | | | |
| I1KVR2 | Uncharacterized protein | 42.80 | 8 | 1 | 10 | 11 | 243 | 28.3 | 9.95 |
| | OS = Glycine max PE = 3 SV = 1 - | | | | | | | | |
| | [I1KVR2_SOYBN] | | | | | | | | |
| I1KZJ1 | Uncharacterized protein | 20.15 | 7 | 6 | 7 | 8 | 392 | 43.0 | 5.54 |
| | OS = Glycine max PE = 3 SV = 1 - | | | | | | | | |
| | [I1KZJ1_SOYBN] | | | | | | | | |
| I1KZJ9 | Uncharacterized protein | 8.83 | 2 | 6 | 6 | 6 | 725 | 81.0 | 7.39 |
| | OS = Glycine max PE = 3 SV = 1 - | | | | | | | | |
| | [I1KZJ9_SOYBN] | | | | | | | | |
| I1KZK2 | Uncharacterized protein | 10.47 | 3 | 6 | 6 | 6 | 535 | 58.9 | 5.85 |
| | OS = Glycine max PE = 3 SV = 1 - | | | | | | | | |
| | [I1KZK2_SOYBN] | | | | | | | | |
| I1KZN9 | Uncharacterized protein | 17.24 | 13 | 1 | 2 | 3 | 116 | 13.2 | 10.15 |
| | OS = Glycine max PE = 3 SV = 1 - | | | | | | | | |
| | [I1KZN9_SOYBN] | | | | | | | | |
| I1KZW7 | Uncharacterized protein | 8.01 | 5 | 2 | 4 | 5 | 562 | 61.4 | 7.03 |
| | OS = Glycine max PE = 3 SV = 1 - | | | | | | | | |
| | [I1KZW7_SOYBN] | | | | | | | | |
| I1L1Q8 | Uncharacterized protein | 17.68 | 4 | 1 | 7 | 7 | 509 | 54.4 | 6.04 |
| | OS = Glycine max PE = 3 SV = 1 - | | | | | | | | |
| | [I1L1Q8_SOYBN] | | | | | | | | |
| I1L314 | Uncharacterized protein | 10.01 | 5 | 1 | 6 | 7 | 699 | 80.3 | 5.06 |
| | OS = Glycine max PE = 3 SV = 1 - | | | | | | | | |
| | [I1L314_SOYBN] | | | | | | | | |
| I1L8A9 | Uncharacterized protein | 5.72 | 3 | 2 | 2 | 2 | 297 | 30.9 | 10.61 |
| | OS = Glycine max PE = 3 SV = 1 - | | | | | | | | |
| | [I1L8A9_SOYBN] | | | | | | | | |
| I1L9G9 | Uncharacterized protein | 21.58 | 2 | 1 | 3 | 3 | 139 | 15.8 | 10.23 |
| | OS = Glycine max PE = 3 SV = 1 - | | | | | | | | |
| | [I1L9G9_SOYBN] | | | | | | | | |
| I1LBD0 | Uncharacterized protein | 8.28 | 2 | 4 | 4 | 4 | 447 | 51.3 | 6.43 |
| | OS = Glycine max PE = 3 SV = 1 - | | | | | | | | |
| | [I1LBD0_SOYBN] | | | | | | | | |
| I1LDR2 | Uncharacterized protein | 31.25 | 9 | 2 | 11 | 14 | 448 | 50.4 | 4.83 |
| | OS = Glycine max PE = 3 SV = 1 - | | | | | | | | |
| | [I1LDR2_SOYBN] | | | | | | | | |
| I1LEI9 | Uncharacterized protein | 2.86 | 1 | 1 | 1 | 1 | 594 | 62.6 | 6.76 |
| | OS = Glycine max PE = 3 SV = 1 - | | | | | | | | |
| | [I1LEI9_SOYBN] | | | | | | | | |
| I1LEX2 | Uncharacterized protein | 3.35 | 1 | 1 | 2 | 2 | 925 | 103.6 | 6.44 |
| | OS = Glycine max PE = 3 SV = 1 - | | | | | | | | |
| | [I1LEX2_SOYBN] | | | | | | | | |
| I1LF60 | Uncharacterized protein | 9.84 | 6 | 4 | 4 | 4 | 488 | 54.2 | 5.02 |
| | OS = Glycine max PE = 3 SV = 1 - | | | | | | | | |
| | [I1LF60_SOYBN] | | | | | | | | |
| I1LFG6 | Uncharacterized protein | 23.90 | 1 | 3 | 11 | 13 | 636 | 67.6 | 7.50 |
| | OS = Glycine max PE = 3 SV = 1 - | | | | | | | | |
| | [I1LFG6_SOYBN] | | | | | | | | |
| I1LFQ4 | Uncharacterized protein | 36.89 | 3 | 3 | 4 | 4 | 122 | 13.7 | 9.58 |
| | OS = Glycine max PE = 3 SV = 1 - | | | | | | | | |
| | [I1LFQ4_SOYBN] | | | | | | | | |
| I1LGR6 | Uncharacterized protein | 7.47 | 2 | 7 | 7 | 7 | 1125 | 127.3 | 6.71 |
| | OS = Glycine max PE = 3 SV = 1 - | | | | | | | | |
| | [I1LGR6_SOYBN] | | | | | | | | |
| I1LHN4 | Uncharacterized protein | 5.18 | 3 | 1 | 3 | 3 | 657 | 73.2 | 5.85 |
| | OS = Glycine max PE = 3 SV = 1 - | | | | | | | | |
| | [I1LHN4_SOYBN] | | | | | | | | |
| I1LIG6 | Uncharacterized protein | 2.95 | 2 | 1 | 1 | 1 | 577 | 64.8 | 7.55 |
| | OS = Glycine max PE = 3 SV = 1 - | | | | | | | | |
| | [I1LIG6_SOYBN] | | | | | | | | |
| I1LIN0 | Uncharacterized protein | 6.24 | 4 | 3 | 3 | 3 | 513 | 56.3 | 7.83 |
| | OS = Glycine max PE = 3 SV = 1 - | | | | | | | | |
| | [I1LIN0_SOYBN] | | | | | | | | |
| I1LK39 | Uncharacterized protein | 6.25 | 3 | 1 | 1 | 1 | 160 | 17.5 | 4.46 |
| | OS = Glycine max PE = 3 SV = 1 - | | | | | | | | |
| | [I1LK39_SOYBN] | | | | | | | | |
| I1LKU1 | Uncharacterized protein | 14.42 | 2 | 5 | 5 | 5 | 527 | 57.0 | 5.80 |
| | OS = Glycine max PE = 3 SV = 1 - | | | | | | | | |
| | [I1LKU1_SOYBN] | | | | | | | | |
| I1LLZ2 | Uncharacterized protein | 4.60 | 13 | 1 | 2 | 3 | 413 | 46.4 | 5.85 |
| | OS = Glycine max PE = 3 SV = 1 - | | | | | | | | |
| | [I1LLZ2_SOYBN] | | | | | | | | |
| I1LNE6 | Uncharacterized protein | 8.22 | 2 | 4 | 4 | 4 | 535 | 59.2 | 5.85 |
| | OS = Glycine max PE = 3 SV = 1 - | | | | | | | | |
| | [I1LNE6_SOYBN] | | | | | | | | |
| I1LP45 | Uncharacterized protein | 16.03 | 7 | 5 | 5 | 8 | 368 | 39.3 | 5.73 |
| | OS = Glycine max PE = 3 SV = 1 - | | | | | | | | |
| | [I1LP45_SOYBN] | | | | | | | | |
| I1LPI1 | Uncharacterized protein | 12.11 | 2 | 2 | 2 | 2 | 190 | 20.0 | 5.68 |
| | OS = Glycine max PE = 3 SV = 1 - | | | | | | | | |
| | [I1LPI1_SOYBN] | | | | | | | | |
| I1LQR4 | Uncharacterized protein | 33.13 | 9 | 2 | 17 | 23 | 649 | 71.0 | 5.21 |
| | OS = Glycine max PE = 3 SV = 1 - | | | | | | | | |
| | [I1LQR4_SOYBN] | | | | | | | | |
| I1LTT3 | Uncharacterized protein | 30.63 | 3 | 2 | 2 | 3 | 111 | 11.2 | 4.37 |
| | OS = Glycine max PE = 3 SV = 1 - | | | | | | | | |
| | [I1LTT3_SOYBN] | | | | | | | | |
| I1LUM3 | Uncharacterized protein | 21.92 | 9 | 1 | 4 | 4 | 260 | 29.3 | 4.82 |
| | OS = Glycine max PE = 3 SV = 1 - | | | | | | | | |
| | [I1LUM3_SOYBN] | | | | | | | | |
| I1LXD1 | Uncharacterized protein | 49.58 | 2 | 6 | 12 | 44 | 357 | 38.7 | 7.94 |
| | OS = Glycine max PE = 3 SV = 1 - | | | | | | | | |
| | [I1LXD1_SOYBN] | | | | | | | | |
| I1LXK6 | Uncharacterized protein | 2.36 | 2 | 2 | 2 | 2 | 891 | 99.0 | 5.45 |
| | OS = Glycine max PE = 3 SV = 1 - | | | | | | | | |
| | [I1LXK6_SOYBN] | | | | | | | | |
| I1LXY1 | Uncharacterized protein | 25.28 | 3 | 11 | 18 | 23 | 803 | 89.0 | 6.77 |
| | OS = Glycine max PE = 3 SV = 1 - | | | | | | | | |
| | [I1LXY1_SOYBN] | | | | | | | | |
| I1LY10 | Uncharacterized protein | 21.80 | 20 | 6 | 6 | 6 | 344 | 39.6 | 4.41 |
| | OS = Glycine max PE = 3 SV = 1 - | | | | | | | | |
| | [I1LY10_SOYBN] | | | | | | | | |
| I1M005 | Uncharacterized protein | 56.69 | 3 | 3 | 19 | 59 | 381 | 41.9 | 7.59 |
| | OS = Glycine max PE = 3 SV = 1 - | | | | | | | | |
| | [I1M005_SOYBN] | | | | | | | | |
| I1M1V8 | Uncharacterized protein | 4.24 | 7 | 3 | 3 | 3 | 589 | 63.4 | 6.11 |
| | OS = Glycine max PE = 3 SV = 1 - | | | | | | | | |
| | [I1M1V8_SOYBN] | | | | | | | | |
| I1M290 | Uncharacterized protein | 22.60 | 4 | 1 | 3 | 5 | 146 | 16.7 | 10.92 |
| | OS = Glycine max PE = 3 SV = 1 - | | | | | | | | |
| | [I1M290_SOYBN] | | | | | | | | |
| I1M2K9 | Uncharacterized protein | 6.23 | 5 | 4 | 4 | 4 | 674 | 72.4 | 5.87 |
| | OS = Glycine max PE = 3 SV = 1 - | | | | | | | | |
| | [I1M2K9_SOYBN] | | | | | | | | |
| I1M3R5 | Uncharacterized protein | 14.17 | 12 | 1 | 5 | 5 | 367 | 39.6 | 9.85 |
| | OS = Glycine max PE = 3 SV = 1 - | | | | | | | | |
| | [I1M3R5_SOYBN] | | | | | | | | |
| I1M561 | Uncharacterized protein | 37.46 | 15 | 8 | 10 | 12 | 331 | 35.4 | 5.45 |
| | OS = Glycine max PE = 3 SV = 1 - | | | | | | | | |
| | [I1M561_SOYBN] | | | | | | | | |
| I1M5L8 | Uncharacterized protein | 12.35 | 5 | 9 | 9 | 9 | 858 | 94.9 | 5.21 |
| | OS = Glycine max PE = 3 SV = 1 - | | | | | | | | |
| | [I1M5L8_SOYBN] | | | | | | | | |
| I1M5P2 | Uncharacterized protein | 13.31 | 9 | 2 | 2 | 2 | 278 | 30.7 | 5.85 |
| | OS = Glycine max PE = 3 SV = 1 - | | | | | | | | |
| | [I1M5P2_SOYBN] | | | | | | | | |
| I1M6P9 | Uncharacterized protein | 11.50 | 5 | 4 | 4 | 4 | 461 | 49.8 | 9.16 |
| | OS = Glycine max PE = 3 SV = 1 - | | | | | | | | |
| | [I1M6P9_SOYBN] | | | | | | | | |
| I1M786 | Uncharacterized protein | 47.98 | 2 | 1 | 7 | 11 | 223 | 24.2 | 4.68 |
| | OS = Glycine max PE = 3 SV = 1 - | | | | | | | | |
| | [I1M786_SOYBN] | | | | | | | | |
| I1M7M8 | Uncharacterized protein | 6.62 | 4 | 2 | 2 | 2 | 317 | 35.0 | 6.81 |
| | OS = Glycine max PE = 3 SV = 1 - | | | | | | | | |
| | [I1M7M8_SOYBN] | | | | | | | | |
| I1M7W9 | Uncharacterized protein | 22.97 | 2 | 2 | 3 | 3 | 222 | 23.4 | 8.13 |
| | OS = Glycine max PE = 3 SV = 1 - | | | | | | | | |
| | [I1M7W9_SOYBN] | | | | | | | | |
| I1MAE6 | Uncharacterized protein | 30.79 | 2 | 5 | 11 | 14 | 380 | 40.8 | 6.52 |
| | OS = Glycine max PE = 3 SV = 1 - | | | | | | | | |
| | [I1MAE6_SOYBN] | | | | | | | | |
| I1MBL5 | Uncharacterized protein | 1.98 | 3 | 1 | 1 | 1 | 505 | 57.9 | 8.75 |
| | OS = Glycine max PE = 3 SV = 1 - | | | | | | | | |
| | [I1MBL5_SOYBN] | | | | | | | | |
| I1MBN8 | Uncharacterized protein | 4.06 | 3 | 2 | 2 | 2 | 566 | 60.6 | 6.54 |
| | OS = Glycine max PE = 3 SV = 1 - | | | | | | | | |
| | [I1MBN8_SOYBN] | | | | | | | | |
| I1MCE2 | Uncharacterized protein | 2.81 | 2 | 1 | 1 | 1 | 499 | 55.2 | 9.17 |
| | OS = Glycine max PE = 3 SV = 1 - | | | | | | | | |
| | [I1MCE2_SOYBN] | | | | | | | | |
| I1MHH0 | Uncharacterized protein | 19.55 | 6 | 1 | 7 | 7 | 491 | 52.4 | 5.85 |
| | OS = Glycine max PE = 3 SV = 1 - | | | | | | | | |
| | [I1MHH0_SOYBN] | | | | | | | | |
| I1MI18 | Uncharacterized protein | 3.01 | 1 | 1 | 1 | 1 | 365 | 40.1 | 8.87 |
| | OS = Glycine max PE = 3 SV = 1 - | | | | | | | | |
| | [I1MI18_SOYBN] | | | | | | | | |
| I1MJ28 | Uncharacterized protein | 20.47 | 3 | 11 | 11 | 11 | 591 | 62.8 | 5.87 |
| | OS = Glycine max PE = 3 SV = 1 - | | | | | | | | |
| | [I1MJ28_SOYBN] | | | | | | | | |
| I1MJN2 | Uncharacterized protein | 16.97 | 8 | 2 | 6 | 6 | 389 | 42.1 | 9.70 |
| | OS = Glycine max PE = 3 SV = 1 - | | | | | | | | |
| | [I1MJN2_SOYBN] | | | | | | | | |
| I1MJU7 | Uncharacterized protein | 16.55 | 3 | 11 | 11 | 13 | 689 | 73.7 | 5.34 |
| | OS = Glycine max PE = 3 SV = 1 - | | | | | | | | |
| | [I1MJU7_SOYBN] | | | | | | | | |
| I1ML11 | Uncharacterized protein | 18.51 | 3 | 2 | 7 | 10 | 443 | 49.1 | 5.22 |
| | OS = Glycine max PE = 3 SV = 1 - | | | | | | | | |
| | [I1ML11_SOYBN] | | | | | | | | |
| I1MNX4 | Uncharacterized protein | 13.97 | 2 | 7 | 7 | 7 | 501 | 54.0 | 5.85 |
| | OS = Glycine max PE = 3 SV = 1 - | | | | | | | | |
| | [I1MNX4_SOYBN] | | | | | | | | |
| I1MQ32 | Uncharacterized protein | 3.79 | 2 | 1 | 1 | 1 | 290 | 31.0 | 6.21 |
| | OS = Glycine max PE = 3 SV = 1 - | | | | | | | | |
| | [I1MQ32_SOYBN] | | | | | | | | |
| I1MQD2 | Uncharacterized protein | 66.67 | 3 | 6 | 6 | 18 | 108 | 12.2 | 6.04 |
| | OS = Glycine max PE = 3 SV = 1 - | | | | | | | | |
| | [I1MQD2_SOYBN] | | | | | | | | |
| I1MR37 | Uncharacterized protein | 5.64 | 4 | 1 | 3 | 3 | 514 | 55.4 | 6.95 |
| | OS = Glycine max PE = 3 SV = 1 - | | | | | | | | |
| | [I1MR37_SOYBN] | | | | | | | | |
| I1MT10 | Uncharacterized protein | 37.98 | 8 | 3 | 20 | 25 | 645 | 70.8 | 5.39 |
| | OS = Glycine max PE = 3 SV = 1 - | | | | | | | | |
| | [I1MT10_SOYBN] | | | | | | | | |
| I1MT23 | Uncharacterized protein | 27.89 | 3 | 12 | 13 | 15 | 570 | 59.8 | 7.14 |
| | OS = Glycine max PE = 3 SV = 1 - | | | | | | | | |
| | [I1MT23_SOYBN] | | | | | | | | |
| I1MT35 | Uncharacterized protein | 15.29 | 3 | 6 | 6 | 6 | 497 | 53.1 | 7.15 |
| | OS = Glycine max PE = 3 SV = 1 - | | | | | | | | |
| | [I1MT35_SOYBN] | | | | | | | | |
| I1MU00 | Uncharacterized protein | 23.90 | 7 | 5 | 5 | 6 | 159 | 17.8 | 10.76 |
| | OS = Glycine max PE = 3 SV = 1 - | | | | | | | | |
| | [I1MU00_SOYBN] | | | | | | | | |
| I1MUN0 | Uncharacterized protein | 8.33 | 3 | 1 | 1 | 1 | 180 | 19.9 | 8.09 |
| | OS = Glycine max PE = 3 SV = 1 - | | | | | | | | |
| | [I1MUN0_SOYBN] | | | | | | | | |
| I1MUR2 | Uncharacterized protein | 4.14 | 2 | 1 | 1 | 1 | 338 | 36.7 | 6.37 |
| | OS = Glycine max PE = 3 SV = 1 - | | | | | | | | |
| | [I1MUR2_SOYBN] | | | | | | | | |
| I1N5S0 | Uncharacterized protein | 65.30 | 5 | 4 | 20 | 50 | 389 | 42.8 | 6.77 |
| | OS = Glycine max PE = 3 SV = 1 - | | | | | | | | |
| | [I1N5S0_SOYBN] | | | | | | | | |
| I1N7G4 | Uncharacterized protein | 20.27 | 1 | 8 | 9 | 9 | 582 | 60.8 | 6.80 |
| | OS = Glycine max PE = 3 SV = 1 - | | | | | | | | |
| | [I1N7G4_SOYBN] | | | | | | | | |
| I1N954 | Uncharacterized protein | 43.09 | 8 | 2 | 4 | 6 | 123 | 13.8 | 9.47 |
| | OS = Glycine max PE = 3 SV = 1 - | | | | | | | | |
| | [I1N954_SOYBN] | | | | | | | | |
| I1NAI7 | Uncharacterized protein | 26.13 | 7 | 1 | 9 | 18 | 444 | 47.6 | 5.57 |
| | OS = Glycine max PE = 3 SV = 1 - | | | | | | | | |
| | [I1NAI7_SOYBN] | | | | | | | | |
| I1NBM7 | Uncharacterized protein | 3.94 | 3 | 2 | 2 | 2 | 508 | 55.5 | 6.73 |
| | OS = Glycine max PE = 3 SV = 1 - | | | | | | | | |
| | [I1NBM7_SOYBN] | | | | | | | | |
| I1ND96 | Uncharacterized protein | 43.30 | 4 | 4 | 4 | 5 | 97 | 10.6 | 7.44 |
| | OS = Glycine max PE = 3 SV = 1 - | | | | | | | | |
| | [I1ND96_SOYBN] | | | | | | | | |
| I1NDC6 | Uncharacterized protein | 13.55 | 1 | 1 | 1 | 1 | 155 | 17.0 | 4.94 |
| | OS = Glycine max PE = 3 SV = 1 - | | | | | | | | |
| | [I1NDC6_SOYBN] | | | | | | | | |
| I1NEH3 | Uncharacterized protein | 13.45 | 6 | 5 | 5 | 5 | 461 | 48.4 | 7.58 |
| | OS = Glycine max PE = 3 SV = 1 - | | | | | | | | |
| | [I1NEH3_SOYBN] | | | | | | | | |
| I1NES7 | Uncharacterized protein | 2.46 | 2 | 1 | 1 | 1 | 732 | 83.1 | 7.27 |
| | OS = Glycine max PE = 3 SV = 1 - | | | | | | | | |
| | [I1NES7_SOYBN] | | | | | | | | |
| I1NF03 | Uncharacterized protein | 38.80 | 1 | 3 | 14 | 17 | 451 | 52.0 | 4.64 |
| | OS = Glycine max PE = 3 SV = 1 - | | | | | | | | |
| | [I1NF03_SOYBN] | | | | | | | | |
| I1NFH5 | Uncharacterized protein | 2.54 | 2 | 1 | 1 | 1 | 472 | 48.9 | 8.70 |
| | OS = Glycine max PE = 3 SV = 1 - | | | | | | | | |
| | [I1NFH5_SOYBN] | | | | | | | | |
| I1NH37 | Uncharacterized protein | 34.45 | 10 | 15 | 15 | 22 | 389 | 44.4 | 10.15 |
| | OS = Glycine max PE = 3 SV = 1 - | | | | | | | | |
| | [I1NH37_SOYBN] | | | | | | | | |
| I1NHW4 | Uncharacterized protein | 5.74 | 3 | 3 | 3 | 3 | 575 | 61.2 | 5.90 |
| | OS = Glycine max PE = 3 SV = 1 - | | | | | | | | |
| | [I1NHW4_SOYBN] | | | | | | | | |
| I1NIG1 | Uncharacterized protein | 8.27 | 3 | 2 | 2 | 3 | 254 | 27.1 | 8.07 |
| | OS = Glycine max PE = 3 SV = 1 - | | | | | | | | |
| | [I1NIG1_SOYBN] | | | | | | | | |
| I1NJ85 | Uncharacterized protein | 31.76 | 2 | 6 | 14 | 15 | 636 | 67.4 | 6.98 |
| | OS = Glycine max PE = 3 SV = 1 - | | | | | | | | |
| | [I1NJ85_SOYBN] | | | | | | | | |
| I1NJA4 | Uncharacterized protein | 9.23 | 9 | 4 | 4 | 4 | 531 | 58.1 | 7.02 |
| | OS = Glycine max PE = 3 SV = 1 - | | | | | | | | |
| | [I1NJA4_SOYBN] | | | | | | | | |
| K7K4T0 | Uncharacterized protein | 9.00 | 7 | 1 | 2 | 2 | 289 | 30.9 | 6.58 |
| | OS = Glycine max PE = 3 SV = 1 - | | | | | | | | |
| | [K7K4T0_SOYBN] | | | | | | | | |
| K7K7B7 | Uncharacterized protein | 29.11 | 8 | 1 | 13 | 18 | 560 | 61.6 | 5.24 |
| | OS = Glycine max PE = 3 SV = 1 - | | | | | | | | |
| | [K7K7B7_SOYBN] | | | | | | | | |
| K7KA83 | Uncharacterized protein | 2.29 | 5 | 2 | 2 | 2 | 1092 | 120.5 | 5.25 |
| | OS = Glycine max PE = 3 SV = 1 - | | | | | | | | |
| | [K7KA83_SOYBN] | | | | | | | | |
| K7KEW2 | Uncharacterized protein | 0.83 | 2 | 1 | 1 | 1 | 964 | 111.5 | 6.29 |
| | OS = Glycine max PE = 3 SV = 1 - | | | | | | | | |
| | [K7KEW2_SOYBN] | | | | | | | | |
| K7KFI7 | Uncharacterized protein | 31.20 | 3 | 4 | 4 | 7 | 125 | 13.4 | 10.01 |
| | OS = Glycine max PE = 3 SV = 1 - | | | | | | | | |
| | [K7KFI7_SOYBN] | | | | | | | | |
| K7KFS7 | Uncharacterized protein | 6.06 | 2 | 1 | 1 | 1 | 198 | 21.0 | 9.82 |
| | OS = Glycine max PE = 3 SV = 1 - | | | | | | | | |
| | [K7KFS7_SOYBN] | | | | | | | | |
| K7KH80 | Uncharacterized protein | 1.74 | 11 | 1 | 1 | 1 | 862 | 94.7 | 6.51 |
| | OS = Glycine max PE = 3 SV = 1 - | | | | | | | | |
| | [K7KH80_SOYBN] | | | | | | | | |
| K7KMK9 | Uncharacterized protein | 5.36 | 8 | 1 | 1 | 1 | 317 | 35.0 | 5.82 |
| | OS = Glycine max PE = 3 SV = 1 - | | | | | | | | |
| | [K7KMK9_SOYBN] | | | | | | | | |
| K7L2A2 | Uncharacterized protein | 6.94 | 7 | 1 | 3 | 4 | 620 | 68.9 | 6.05 |
| | OS = Glycine max PE = 3 SV = 1 - | | | | | | | | |
| | [K7L2A2_SOYBN] | | | | | | | | |
| K7L353 | Uncharacterized protein | 23.44 | 5 | 1 | 3 | 3 | 128 | 15.0 | 10.17 |
| | OS = Glycine max PE = 3 SV = 1 - | | | | | | | | |
| | [K7L353_SOYBN] | | | | | | | | |
| K7L3M0 | Uncharacterized protein | 43.04 | 9 | 1 | 5 | 7 | 158 | 16.8 | 4.88 |
| | OS = Glycine max PE = 3 SV = 1 - | | | | | | | | |
| | [K7L3M0_SOYBN] | | | | | | | | |
| K7L505 | Uncharacterized protein | 35.16 | 1 | 3 | 3 | 5 | 91 | 9.9 | 7.21 |
| | OS = Glycine max PE = 3 SV = 1 - | | | | | | | | |
| | [K7L505_SOYBN] | | | | | | | | |
| K7L5A8 | Uncharacterized protein | 2.50 | 3 | 1 | 1 | 1 | 440 | 47.4 | 7.20 |
| | OS = Glycine max PE = 3 SV = 1 - | | | | | | | | |
| | [K7L5A8_SOYBN] | | | | | | | | |
| K7LI86 | Uncharacterized protein | 1.64 | 2 | 1 | 1 | 1 | 669 | 75.5 | 6.89 |
| | OS = Glycine max PE = 3 SV = 1 - | | | | | | | | |
| | [K7LI86_SOYBN] | | | | | | | | |
| K7LMN7 | Uncharacterized protein | 5.05 | 6 | 1 | 1 | 1 | 198 | 20.9 | 8.48 |
| | OS = Glycine max PE = 3 SV = 1 - | | | | | | | | |
| | [K7LMN7_SOYBN] | | | | | | | | |
| K7LS95 | Uncharacterized protein | 4.85 | 9 | 1 | 1 | 1 | 165 | 18.5 | 8.40 |
| | OS = Glycine max PE = 3 SV = 1 - | | | | | | | | |
| | [K7LS95_SOYBN] | | | | | | | | |
| K7LVG8 | Uncharacterized protein | 22.83 | 11 | 4 | 4 | 4 | 254 | 27.1 | 7.36 |
| | OS = Glycine max PE = 3 SV = 1 - | | | | | | | | |
| | [K7LVG8_SOYBN] | | | | | | | | |
| K7LZH1 | Uncharacterized protein | 25.32 | 8 | 1 | 13 | 18 | 632 | 69.2 | 5.25 |
| | OS = Glycine max PE = 3 SV = 1 - | | | | | | | | |
| | [K7LZH1_SOYBN] | | | | | | | | |
| K7M9L4 | Uncharacterized protein | 6.55 | 3 | 6 | 6 | 7 | 1161 | 126.7 | 5.43 |
| | OS = Glycine max PE = 3 SV = 1 - | | | | | | | | |
| | [K7M9L4_SOYBN] | | | | | | | | |
| K7MBV6 | Uncharacterized protein | 30.88 | 11 | 3 | 8 | 9 | 272 | 31.9 | 10.05 |
| | OS = Glycine max PE = 3 SV = 1 - | | | | | | | | |
| | [K7MBV6_SOYBN] | | | | | | | | |
| K7MNM4 | Uncharacterized protein | 3.59 | 3 | 1 | 1 | 1 | 446 | 48.7 | 5.08 |
| | OS = Glycine max PE = 3 SV = 1 - | | | | | | | | |
| | [K7MNM4_SOYBN] | | | | | | | | |
| K7MP24 | Uncharacterized protein | 17.34 | 10 | 12 | 13 | 15 | 813 | 93.1 | 5.00 |
| | OS = Glycine max PE = 3 SV = 1 - | | | | | | | | |
| | [K7MP24_SOYBN] | | | | | | | | |
| K7MQ21 | Uncharacterized protein | 10.78 | 27 | 1 | 1 | 1 | 102 | 11.3 | 8.47 |
| | OS = Glycine max PE = 3 SV = 1 - | | | | | | | | |
| | [K7MQ21_SOYBN] | | | | | | | | |
| K7MRB1 | Uncharacterized protein | 4.44 | 5 | 1 | 1 | 1 | 180 | 21.0 | 6.70 |
| | OS = Glycine max PE = 3 SV = 1 - | | | | | | | | |
| | [K7MRB1_SOYBN] | | | | | | | | |
| K7MTK7 | Uncharacterized protein | 10.54 | 11 | 2 | 2 | 2 | 294 | 32.4 | 9.09 |
| | OS = Glycine max PE = 3 SV = 1 - | | | | | | | | |
| | [K7MTK7_SOYBN] | | | | | | | | |
| K7MZ15 | Uncharacterized protein | 5.31 | 6 | 2 | 2 | 2 | 452 | 49.9 | 7.34 |
| | OS = Glycine max PE = 3 SV = 1 - | | | | | | | | |
| | [K7MZ15_SOYBN] | | | | | | | | |
| K7MZ77 | Uncharacterized protein | 8.85 | 2 | 1 | 1 | 1 | 113 | 11.2 | 4.41 |
| | OS = Glycine max PE = 3 SV = 1 - | | | | | | | | |
| | [K7MZ77_SOYBN] | | | | | | | | |
| K7N0E2 | Uncharacterized protein | 47.47 | 2 | 2 | 3 | 3 | 99 | 9.8 | 6.00 |
| | OS = Glycine max PE = 3 SV = 1 - | | | | | | | | |
| | [K7N0E2_SOYBN] | | | | | | | | |
| I1J6K4 | Uncharacterized protein | 4.85 | 2 | 3 | 4 | 5 | 928 | 102.9 | 6.70 |
| | OS = Glycine max PE = 3 SV = 2 - | | | | | | | | |
| | [I1J6K4_SOYBN] | | | | | | | | |
| I1JI26 | Uncharacterized protein | 1.40 | 2 | 1 | 1 | 1 | 713 | 77.5 | 6.84 |
| | OS = Glycine max PE = 3 SV = 2 - | | | | | | | | |
| | [I1JI26_SOYBN] | | | | | | | | |
| I1JVV8 | Uncharacterized protein | 1.71 | 1 | 1 | 1 | 1 | 1053 | 117.4 | 6.14 |
| | OS = Glycine max PE = 3 SV = 2 - | | | | | | | | |
| | [I1JVV8_SOYBN] | | | | | | | | |
| I1JYL7 | Uncharacterized protein | 11.29 | 3 | 1 | 1 | 1 | 124 | 14.2 | 7.49 |
| | OS = Glycine max PE = 3 SV = 2 - | | | | | | | | |
| | [I1JYL7_SOYBN] | | | | | | | | |
| I1KCD7 | Uncharacterized protein | 29.24 | 1 | 1 | 21 | 25 | 814 | 90.9 | 5.24 |
| | OS = Glycine max PE = 3 SV = 2 - | | | | | | | | |
| | [I1KCD7_SOYBN] | | | | | | | | |
| I1KM19 | Uncharacterized protein | 54.49 | 1 | 1 | 7 | 7 | 156 | 17.7 | 10.20 |
| | OS = Glycine max PE = 3 SV = 2 - | | | | | | | | |
| | [I1KM19_SOYBN] | | | | | | | | |
| I1KQ70 | Uncharacterized protein | 3.79 | 2 | 3 | 3 | 3 | 792 | 87.5 | 6.58 |
| | OS = Glycine max PE = 3 SV = 2 - | | | | | | | | |
| | [I1KQ70_SOYBN] | | | | | | | | |
| I1KXG9 | Uncharacterized protein | 22.39 | 8 | 1 | 11 | 14 | 536 | 58.4 | 7.55 |
| | OS = Glycine max PE = 3 SV = 2 - | | | | | | | | |
| | [I1KXG9_SOYBN] | | | | | | | | |
| I1L4U3 | Uncharacterized protein | 2.80 | 2 | 2 | 2 | 2 | 787 | 88.4 | 6.30 |
| | OS = Glycine max PE = 3 SV = 2 - | | | | | | | | |
| | [I1L4U3_SOYBN] | | | | | | | | |
| I1LD66 | Uncharacterized protein | 5.88 | 2 | 1 | 1 | 1 | 425 | 44.2 | 9.29 |
| | OS = Glycine max PE = 3 SV = 2 - | | | | | | | | |
| | [I1LD66_SOYBN] | | | | | | | | |
| I1LIK8 | Uncharacterized protein | 1.69 | 2 | 2 | 2 | 2 | 1182 | 134.8 | 6.67 |
| | OS = Glycine max PE = 3 SV = 2 - | | | | | | | | |
| | [I1LIK8_SOYBN] | | | | | | | | |
| I1LR00 | Uncharacterized protein | 22.39 | 12 | 3 | 3 | 3 | 134 | 14.9 | 10.43 |
| | OS = Glycine max PE = 3 SV = 2 - | | | | | | | | |
| | [I1LR00_SOYBN] | | | | | | | | |
| I1MJE1 | Uncharacterized protein | 11.52 | 3 | 4 | 4 | 5 | 495 | 52.8 | 6.32 |
| | OS = Glycine max PE = 3 SV = 2 - | | | | | | | | |
| | [I1MJE1_SOYBN] | | | | | | | | |
| I1N1F9 | Uncharacterized protein | 22.01 | 10 | 1 | 11 | 14 | 536 | 58.6 | 7.15 |
| | OS = Glycine max PE = 3 SV = 2 - | | | | | | | | |
| | [I1N1F9_SOYBN] | | | | | | | | |
| I1NFK8 | Uncharacterized protein | 4.14 | 7 | 3 | 3 | 3 | 748 | 84.8 | 6.52 |
| | OS = Glycine max PE = 3 SV = 2 - | | | | | | | | |
| | [I1NFK8_SOYBN] | | | | | | | | |
| I1J637 | Uncharacterized protein | 21.74 | 9 | 1 | 8 | 10 | 368 | 41.7 | 5.78 |
| | OS = Glycine max PE = 4 SV = 1 - | | | | | | | | |
| | [I1J637_SOYBN] | | | | | | | | |
| I1J7A4 | Uncharacterized protein | 1.79 | 2 | 1 | 1 | 1 | 392 | 43.5 | 4.45 |
| | OS = Glycine max PE = 4 SV = 1 - | | | | | | | | |
| | [I1J7A4_SOYBN] | | | | | | | | |
| I1J9X9 | Uncharacterized protein | 1.99 | 4 | 1 | 1 | 1 | 554 | 62.2 | 6.01 |
| | OS = Glycine max PE = 4 SV = 1 - | | | | | | | | |
| | [I1J9X9_SOYBN] | | | | | | | | |
| I1JBG1 | Uncharacterized protein | 12.04 | 2 | 3 | 3 | 3 | 324 | 35.4 | 8.31 |
| | OS = Glycine max PE = 4 SV = 1 - | | | | | | | | |
| | [I1JBG1_SOYBN] | | | | | | | | |
| I1JC44 | Uncharacterized protein | 3.32 | 3 | 1 | 1 | 1 | 422 | 46.5 | 5.88 |
| | OS = Glycine max PE = 4 SV = 1 - | | | | | | | | |
| | [I1JC44_SOYBN] | | | | | | | | |
| I1JCB6 | Uncharacterized protein | 28.86 | 3 | 5 | 5 | 5 | 298 | 31.9 | 7.66 |
| | OS = Glycine max PE = 4 SV = 1 - | | | | | | | | |
| | [I1JCB6_SOYBN] | | | | | | | | |
| I1JEM1 | Uncharacterized protein | 4.61 | 1 | 1 | 1 | 1 | 217 | 25.3 | 4.81 |
| | OS = Glycine max PE = 4 SV = 1 - | | | | | | | | |
| | [I1JEM1_SOYBN] | | | | | | | | |
| I1JFI7 | Uncharacterized protein | 17.91 | 8 | 2 | 2 | 2 | 134 | 14.3 | 5.92 |
| | OS = Glycine max PE = 4 SV = 1 - | | | | | | | | |
| | [I1JFI7_SOYBN] | | | | | | | | |
| I1JFN4 | Uncharacterized protein | 23.85 | 5 | 2 | 2 | 2 | 109 | 11.5 | 4.59 |
| | OS = Glycine max PE = 4 SV = 1 - | | | | | | | | |
| | [I1JFN4_SOYBN] | | | | | | | | |
| I1JFU9 | Uncharacterized protein | 2.42 | 6 | 1 | 1 | 1 | 455 | 49.5 | 7.03 |
| | OS = Glycine max PE = 4 SV = 1 - | | | | | | | | |
| | [I1JFU9_SOYBN] | | | | | | | | |
| I1JFX0 | Uncharacterized protein | 48.17 | 1 | 6 | 6 | 9 | 191 | 20.7 | 7.17 |
| | OS = Glycine max PE = 4 SV = 1 - | | | | | | | | |
| | [I1JFX0_SOYBN] | | | | | | | | |
| I1JGB2 | Uncharacterized protein | 13.68 | 4 | 3 | 3 | 3 | 285 | 32.1 | 7.05 |
| | OS = Glycine max PE = 4 SV = 1 - | | | | | | | | |
| | [I1JGB2_SOYBN] | | | | | | | | |
| I1JGY9 | Uncharacterized protein | 12.86 | 1 | 5 | 5 | 5 | 451 | 48.6 | 8.43 |
| | OS = Glycine max PE = 4 SV = 1 - | | | | | | | | |
| | [I1JGY9_SOYBN] | | | | | | | | |
| I1JJ05 | Uncharacterized protein | 9.30 | 3 | 2 | 2 | 2 | 258 | 28.0 | 7.87 |
| | OS = Glycine max PE = 4 SV = 1 - | | | | | | | | |
| | [I1JJ05_SOYBN] | | | | | | | | |
| I1JL98 | Uncharacterized protein | 10.00 | 2 | 2 | 2 | 2 | 170 | 18.5 | 5.38 |
| | OS = Glycine max PE = 4 SV = 1 - | | | | | | | | |
| | [I1JL98_SOYBN] | | | | | | | | |
| I1JMQ3 | Uncharacterized protein | 10.33 | 2 | 2 | 2 | 2 | 368 | 39.1 | 6.02 |
| | OS = Glycine max PE = 4 SV = 1 - | | | | | | | | |
| | [I1JMQ3_SOYBN] | | | | | | | | |
| I1JMY1 | Uncharacterized protein | 5.18 | 2 | 2 | 2 | 2 | 676 | 73.8 | 6.24 |
| | OS = Glycine max PE = 4 SV = 1 - | | | | | | | | |
| | [I1JMY1_SOYBN] | | | | | | | | |
| I1JMZ7 | Uncharacterized protein | 2.90 | 2 | 2 | 2 | 2 | 965 | 111.5 | 6.10 |
| | OS = Glycine max PE = 4 SV = 1 - | | | | | | | | |
| | [I1JMZ7_SOYBN] | | | | | | | | |
| I1JNI2 | Uncharacterized protein | 1.29 | 2 | 1 | 1 | 1 | 775 | 89.0 | 9.06 |
| | OS = Glycine max PE = 4 SV = 1 - | | | | | | | | |
| | [I1JNI2_SOYBN] | | | | | | | | |
| I1JP48 | Uncharacterized protein | 6.70 | 4 | 3 | 3 | 4 | 687 | 74.8 | 6.33 |
| | OS = Glycine max PE = 4 SV = 1 - | | | | | | | | |
| | [I1JP48_SOYBN] | | | | | | | | |
| I1JPN4 | Uncharacterized protein | 19.14 | 4 | 1 | 3 | 3 | 324 | 36.0 | 5.55 |
| | OS = Glycine max PE = 4 SV = 1 - | | | | | | | | |
| | [I1JPN4_SOYBN] | | | | | | | | |
| I1JPY8 | Uncharacterized protein | 6.83 | 2 | 1 | 5 | 6 | 820 | 93.4 | 5.88 |
| | OS = Glycine max PE = 4 SV = 1 - | | | | | | | | |
| | [I1JPY8_SOYBN] | | | | | | | | |
| I1JQ73 | Uncharacterized protein | 14.02 | 3 | 1 | 2 | 3 | 214 | 24.0 | 5.41 |
| | OS = Glycine max PE = 4 SV = 1 - | | | | | | | | |
| | [I1JQ73_SOYBN] | | | | | | | | |
| I1JQB4 | Uncharacterized protein | 2.43 | 2 | 1 | 1 | 1 | 453 | 50.1 | 6.46 |
| | OS = Glycine max PE = 4 SV = 1 - | | | | | | | | |
| | [I1JQB4_SOYBN] | | | | | | | | |
| I1JQF4 | Uncharacterized protein | 3.63 | 2 | 1 | 1 | 1 | 413 | 46.5 | 5.31 |
| | OS = Glycine max PE = 4 SV = 1 - | | | | | | | | |
| | [I1JQF4_SOYBN] | | | | | | | | |
| I1JQL0 | Uncharacterized protein | 7.03 | 1 | 1 | 1 | 1 | 185 | 20.5 | 10.27 |
| | OS = Glycine max PE = 4 SV = 1 - | | | | | | | | |
| | [I1JQL0_SOYBN] | | | | | | | | |
| I1JQN0 | Uncharacterized protein | 30.68 | 12 | 1 | 5 | 8 | 176 | 19.4 | 11.43 |
| | OS = Glycine max PE = 4 SV = 1 - | | | | | | | | |
| | [I1JQN0_SOYBN] | | | | | | | | |
| I1JR23 | Uncharacterized protein | 3.95 | 3 | 1 | 1 | 1 | 329 | 36.0 | 4.88 |
| | OS = Glycine max PE = 4 SV = 1 - | | | | | | | | |
| | [I1JR23_SOYBN] | | | | | | | | |
| I1JR27 | Uncharacterized protein | 2.33 | 4 | 2 | 2 | 2 | 903 | 98.7 | 8.46 |
| | OS = Glycine max PE = 4 SV = 1 - | | | | | | | | |
| | [I1JR27_SOYBN] | | | | | | | | |
| I1JR89 | Uncharacterized protein | 6.40 | 3 | 1 | 1 | 1 | 250 | 28.2 | 5.53 |
| | OS = Glycine max PE = 4 SV = 1 - | | | | | | | | |
| | [I1JR89_SOYBN] | | | | | | | | |
| I1JRR6 | Uncharacterized protein | 10.42 | 3 | 1 | 2 | 2 | 240 | 27.2 | 7.59 |
| | OS = Glycine max PE = 4 SV = 1 - | | | | | | | | |
| | [I1JRR6_SOYBN] | | | | | | | | |
| I1JSX8 | Uncharacterized protein | 9.69 | 11 | 4 | 4 | 4 | 547 | 60.6 | 9.39 |
| | OS = Glycine max PE = 4 SV = 1 - | | | | | | | | |
| | [I1JSX8_SOYBN] | | | | | | | | |
| I1JU43 | Uncharacterized protein | 8.71 | 1 | 2 | 2 | 2 | 287 | 30.7 | 9.47 |
| | OS = Glycine max PE = 4 SV = 1 - | | | | | | | | |
| | [I1JU43_SOYBN] | | | | | | | | |
| I1JUJ5 | Uncharacterized protein | 5.28 | 2 | 1 | 1 | 1 | 322 | 35.4 | 5.29 |
| | OS = Glycine max PE = 4 SV = 1 - | | | | | | | | |
| | [I1JUJ5_SOYBN] | | | | | | | | |
| I1JUT9 | Uncharacterized protein | 1.63 | 2 | 1 | 1 | 1 | 983 | 110.3 | 7.05 |
| | OS = Glycine max PE = 4 SV = 1 - | | | | | | | | |
| | [I1JUT9_SOYBN] | | | | | | | | |
| I1JV02 | Uncharacterized protein | 4.67 | 1 | 2 | 2 | 2 | 557 | 61.0 | 5.96 |
| | OS = Glycine max PE = 4 SV = 1 - | | | | | | | | |
| | [I1JV02_SOYBN] | | | | | | | | |
| I1JV69 | Uncharacterized protein | 3.56 | 4 | 1 | 1 | 1 | 337 | 37.6 | 5.59 |
| | OS = Glycine max PE = 4 SV = 1 - | | | | | | | | |
| | [I1JV69_SOYBN] | | | | | | | | |
| I1JVH9 | Uncharacterized protein | 9.05 | 1 | 1 | 1 | 1 | 221 | 25.4 | 6.81 |
| | OS = Glycine max PE = 4 SV = 1 - | | | | | | | | |
| | [I1JVH9_SOYBN] | | | | | | | | |
| I1JVP6 | Uncharacterized protein | 2.63 | 4 | 1 | 1 | 1 | 342 | 36.8 | 4.68 |
| | OS = Glycine max PE = 4 SV = 1 - | | | | | | | | |
| | [I1JVP6_SOYBN] | | | | | | | | |
| I1JW44 | Uncharacterized protein | 12.30 | 2 | 5 | 5 | 5 | 439 | 46.9 | 8.54 |
| | OS = Glycine max PE = 4 SV = 1 - | | | | | | | | |
| | [I1JW44_SOYBN] | | | | | | | | |
| I1JXS8 | Uncharacterized protein | 18.48 | 1 | 2 | 16 | 19 | 990 | 108.9 | 6.49 |
| | OS = Glycine max PE = 4 SV = 1 - | | | | | | | | |
| | [I1JXS8_SOYBN] | | | | | | | | |
| I1JYG3 | Uncharacterized protein | 49.48 | 3 | 4 | 8 | 11 | 192 | 21.7 | 9.50 |
| | OS = Glycine max PE = 4 SV = 1 - | | | | | | | | |
| | [I1JYG3_SOYBN] | | | | | | | | |
| I1JZ72 | Uncharacterized protein | 4.56 | 1 | 1 | 1 | 1 | 417 | 43.2 | 5.59 |
| | OS = Glycine max PE = 4 SV = 1 - | | | | | | | | |
| | [I1JZ72_SOYBN] | | | | | | | | |
| I1K0B3 | Uncharacterized protein | 4.93 | 3 | 1 | 1 | 1 | 426 | 46.6 | 4.77 |
| | OS = Glycine max PE = 4 SV = 1 - | | | | | | | | |
| | [I1K0B3_SOYBN] | | | | | | | | |
| I1K198 | Uncharacterized protein | 18.40 | 2 | 1 | 3 | 4 | 163 | 17.4 | 9.33 |
| | OS = Glycine max PE = 4 SV = 1 - | | | | | | | | |
| | [I1K198_SOYBN] | | | | | | | | |
| I1K1H7 | Uncharacterized protein | 29.63 | 16 | 4 | 4 | 5 | 135 | 15.1 | 6.30 |
| | OS = Glycine max PE = 4 SV = 1 - | | | | | | | | |
| | [I1K1H7_SOYBN] | | | | | | | | |
| I1K2B5 | Uncharacterized protein | 1.59 | 1 | 1 | 1 | 1 | 755 | 79.6 | 6.60 |
| | OS = Glycine max PE = 4 SV = 1 - | | | | | | | | |
| | [I1K2B5_SOYBN] | | | | | | | | |
| I1K3R6 | Uncharacterized protein | 27.87 | 1 | 2 | 10 | 14 | 488 | 54.2 | 5.90 |
| | OS = Glycine max PE = 4 SV = 1 - | | | | | | | | |
| | [I1K3R6_SOYBN] | | | | | | | | |
| I1K4T5 | Uncharacterized protein | 8.55 | 2 | 1 | 1 | 1 | 117 | 13.4 | 8.88 |
| | OS = Glycine max PE = 4 SV = 1 - | | | | | | | | |
| | [I1K4T5_SOYBN] | | | | | | | | |
| I1K547 | Uncharacterized protein | 5.11 | 2 | 2 | 2 | 2 | 372 | 39.5 | 7.36 |
| | OS = Glycine max PE = 4 SV = 1 - | | | | | | | | |
| | [I1K547_SOYBN] | | | | | | | | |
| I1K5F0 | Uncharacterized protein | 6.68 | 5 | 3 | 3 | 3 | 479 | 53.3 | 5.49 |
| | OS = Glycine max PE = 4 SV = 1 - | | | | | | | | |
| | [I1K5F0_SOYBN] | | | | | | | | |
| I1K5L6 | Uncharacterized protein | 31.38 | 4 | 2 | 3 | 3 | 188 | 21.7 | 9.35 |
| | OS = Glycine max PE = 4 SV = 1 - | | | | | | | | |
| | [I1K5L6_SOYBN] | | | | | | | | |
| I1K672 | Uncharacterized protein | 1.38 | 2 | 1 | 1 | 1 | 870 | 96.3 | 4.78 |
| | OS = Glycine max PE = 4 SV = 1 - | | | | | | | | |
| | [I1K672_SOYBN] | | | | | | | | |
| I1K6M2 | Uncharacterized protein | 26.13 | 6 | 5 | 5 | 5 | 222 | 23.8 | 7.87 |
| | OS = Glycine max PE = 4 SV = 1 - | | | | | | | | |
| | [I1K6M2_SOYBN] | | | | | | | | |
| I1K7E6 | Uncharacterized protein | 56.82 | 1 | 1 | 3 | 5 | 88 | 9.4 | 5.20 |
| | OS = Glycine max PE = 4 SV = 1 - | | | | | | | | |
| | [I1K7E6_SOYBN] | | | | | | | | |
| I1K7H1 | Uncharacterized protein | 6.38 | 5 | 3 | 3 | 3 | 392 | 45.1 | 8.73 |
| | OS = Glycine max PE = 4 SV = 1 - | | | | | | | | |
| | [I1K7H1_SOYBN] | | | | | | | | |
| I1K7N6 | Uncharacterized protein | 9.40 | 1 | 1 | 1 | 1 | 117 | 13.0 | 5.88 |
| | OS = Glycine max PE = 4 SV = 1 - | | | | | | | | |
| | [I1K7N6_SOYBN] | | | | | | | | |
| I1KA65 | Uncharacterized protein | 20.27 | 4 | 1 | 1 | 1 | 74 | 7.9 | 8.76 |
| | OS = Glycine max PE = 4 SV = 1 - | | | | | | | | |
| | [I1KA65_SOYBN] | | | | | | | | |
| I1KAG7 | Uncharacterized protein | 1.56 | 2 | 1 | 1 | 1 | 512 | 55.7 | 5.06 |
| | OS = Glycine max PE = 4 SV = 1 - | | | | | | | | |
| | [I1KAG7_SOYBN] | | | | | | | | |
| I1KAK6 | Uncharacterized protein | 29.11 | 5 | 6 | 6 | 6 | 237 | 27.4 | 6.42 |
| | OS = Glycine max PE = 4 SV = 1 - | | | | | | | | |
| | [I1KAK6_SOYBN] | | | | | | | | |
| I1KAY3 | Uncharacterized protein | 17.25 | 3 | 4 | 4 | 4 | 255 | 27.7 | 6.54 |
| | OS = Glycine max PE = 4 SV = 1 - | | | | | | | | |
| | [I1KAY3_SOYBN] | | | | | | | | |
| I1KB25 | Uncharacterized protein | 17.94 | 8 | 3 | 3 | 3 | 223 | 24.2 | 4.45 |
| | OS = Glycine max PE = 4 SV = 1 - | | | | | | | | |
| | [I1KB25_SOYBN] | | | | | | | | |
| I1KBM2 | Uncharacterized protein | 2.14 | 1 | 1 | 1 | 1 | 515 | 56.6 | 7.18 |
| | OS = Glycine max PE = 4 SV = 1 - | | | | | | | | |
| | [I1KBM2_SOYBN] | | | | | | | | |
| I1KBV2 | Uncharacterized protein | 20.61 | 1 | 2 | 18 | 21 | 990 | 108.7 | 6.43 |
| | OS = Glycine max PE = 4 SV = 1 - | | | | | | | | |
| | [I1KBV2_SOYBN] | | | | | | | | |
| I1KCI8 | Uncharacterized protein | 2.07 | 1 | 1 | 1 | 1 | 531 | 57.9 | 9.28 |
| | OS = Glycine max PE = 4 SV = 1 - | | | | | | | | |
| | [I1KCI8_SOYBN] | | | | | | | | |
| I1KD10 | Uncharacterized protein | 9.84 | 9 | 2 | 2 | 2 | 254 | 28.0 | 10.14 |
| | OS = Glycine max PE = 4 SV = 1 - | | | | | | | | |
| | [I1KD10_SOYBN] | | | | | | | | |
| I1KDB2 | Uncharacterized protein | 2.35 | 1 | 1 | 1 | 1 | 426 | 47.3 | 6.54 |
| | OS = Glycine max PE = 4 SV = 1 - | | | | | | | | |
| | [I1KDB2_SOYBN] | | | | | | | | |
| I1KDR3 | Uncharacterized protein | 6.00 | 5 | 2 | 2 | 2 | 450 | 48.8 | 5.62 |
| | OS = Glycine max PE = 4 SV = 1 - | | | | | | | | |
| | [I1KDR3_SOYBN] | | | | | | | | |
| I1KDX9 | Uncharacterized protein | 6.51 | 5 | 1 | 1 | 1 | 169 | 18.4 | 9.25 |
| | OS = Glycine max PE = 4 SV = 1 - | | | | | | | | |
| | [I1KDX9_SOYBN] | | | | | | | | |
| I1KEA0 | Uncharacterized protein | 7.29 | 1 | 1 | 2 | 2 | 343 | 37.8 | 6.55 |
| | OS = Glycine max PE = 4 SV = 1 - | | | | | | | | |
| | [I1KEA0_SOYBN] | | | | | | | | |
| I1KEY6 | Uncharacterized protein | 12.20 | 2 | 1 | 9 | 9 | 885 | 97.3 | 5.19 |
| | OS = Glycine max PE = 4 SV = 1 - | | | | | | | | |
| | [I1KEY6_SOYBN] | | | | | | | | |
| I1KHA2 | Uncharacterized protein | 2.26 | 2 | 1 | 1 | 1 | 399 | 44.5 | 8.87 |
| | OS = Glycine max PE = 4 SV = 1 - | | | | | | | | |
| | [I1KHA2_SOYBN] | | | | | | | | |
| I1KL02 | Uncharacterized protein | 11.67 | 1 | 1 | 1 | 1 | 120 | 12.6 | 8.40 |
| | OS = Glycine max PE = 4 SV = 1 - | | | | | | | | |
| | [I1KL02_SOYBN] | | | | | | | | |
| I1KL55 | Uncharacterized protein | 9.80 | 2 | 3 | 3 | 3 | 408 | 45.6 | 6.47 |
| | OS = Glycine max PE = 4 SV = 1 - | | | | | | | | |
| | [I1KL55_SOYBN] | | | | | | | | |
| I1KM07 | Uncharacterized protein | 1.80 | 1 | 1 | 1 | 1 | 556 | 60.0 | 8.73 |
| | OS = Glycine max PE = 4 SV = 1 - | | | | | | | | |
| | [I1KM07_SOYBN] | | | | | | | | |
| I1KM24 | Uncharacterized protein | 5.83 | 9 | 2 | 3 | 3 | 515 | 56.4 | 8.90 |
| | OS = Glycine max PE = 4 SV = 1 - | | | | | | | | |
| | [I1KM24_SOYBN] | | | | | | | | |
| I1KM70 | Uncharacterized protein | 20.59 | 3 | 3 | 3 | 3 | 170 | 19.3 | 5.06 |
| | OS = Glycine max PE = 4 SV = 1 - | | | | | | | | |
| | [I1KM70_SOYBN] | | | | | | | | |
| I1KP14 | Uncharacterized protein | 12.56 | 3 | 5 | 11 | 12 | 995 | 108.7 | 6.48 |
| | OS = Glycine max PE = 4 SV = 1 - | | | | | | | | |
| | [I1KP14_SOYBN] | | | | | | | | |
| I1KPH4 | Uncharacterized protein | 15.43 | 11 | 3 | 3 | 3 | 188 | 22.0 | 5.66 |
| | OS = Glycine max PE = 4 SV = 1 - | | | | | | | | |
| | [I1KPH4_SOYBN] | | | | | | | | |
| I1KQE8 | Uncharacterized protein | 6.56 | 3 | 5 | 5 | 5 | 1006 | 109.5 | 5.36 |
| | OS = Glycine max PE = 4 SV = 1 - | | | | | | | | |
| | [I1KQE8_SOYBN] | | | | | | | | |
| I1KRS9 | Uncharacterized protein | 7.68 | 4 | 4 | 4 | 4 | 599 | 66.1 | 9.00 |
| | OS = Glycine max PE = 4 SV = 1 - | | | | | | | | |
| | [I1KRS9_SOYBN] | | | | | | | | |
| I1KS58 | Uncharacterized protein | 40.08 | 2 | 6 | 14 | 18 | 484 | 53.8 | 6.09 |
| | OS = Glycine max PE = 4 SV = 1 - | | | | | | | | |
| | [I1KS58_SOYBN] | | | | | | | | |
| I1KSQ2 | Uncharacterized protein | 26.65 | 12 | 9 | 9 | 9 | 409 | 45.9 | 6.40 |
| | OS = Glycine max PE = 4 SV = 1 - | | | | | | | | |
| | [I1KSQ2_SOYBN] | | | | | | | | |
| I1KU21 | Uncharacterized protein | 34.52 | 5 | 24 | 24 | 31 | 843 | 94.0 | 6.11 |
| | OS = Glycine max PE = 4 SV = 1 - | | | | | | | | |
| | [I1KU21_SOYBN] | | | | | | | | |
| I1KU40 | Uncharacterized protein | 8.22 | 4 | 3 | 3 | 3 | 377 | 41.1 | 6.71 |
| | OS = Glycine max PE = 4 SV = 1 - | | | | | | | | |
| | [I1KU40_SOYBN] | | | | | | | | |
| I1KUG6 | Uncharacterized protein | 37.67 | 8 | 4 | 11 | 14 | 377 | 41.6 | 5.49 |
| | OS = Glycine max PE = 4 SV = 1 - | | | | | | | | |
| | [I1KUG6_SOYBN] | | | | | | | | |
| I1KV92 | Uncharacterized protein | 1.99 | 8 | 2 | 2 | 3 | 905 | 102.1 | 5.11 |
| | OS = Glycine max PE = 4 SV = 1 - | | | | | | | | |
| | [I1KV92_SOYBN] | | | | | | | | |
| I1KW34 | Uncharacterized protein | 2.89 | 3 | 1 | 1 | 1 | 415 | 45.0 | 5.02 |
| | OS = Glycine max PE = 4 SV = 1 - | | | | | | | | |
| | [I1KW34_SOYBN] | | | | | | | | |
| I1KW97 | Uncharacterized protein | 9.14 | 1 | 2 | 3 | 4 | 383 | 43.0 | 8.22 |
| | OS = Glycine max PE = 4 SV = 1 - | | | | | | | | |
| | [I1KW97_SOYBN] | | | | | | | | |
| I1KWF7 | Uncharacterized protein | 2.17 | 6 | 1 | 1 | 1 | 322 | 36.4 | 4.94 |
| | OS = Glycine max PE = 4 SV = 1 - | | | | | | | | |
| | [I1KWF7_SOYBN] | | | | | | | | |
| I1KWT3 | Uncharacterized protein | 16.08 | 2 | 3 | 4 | 4 | 342 | 37.5 | 6.37 |
| | OS = Glycine max PE = 4 SV = 1 - | | | | | | | | |
| | [I1KWT3_SOYBN] | | | | | | | | |
| I1KXD2 | Uncharacterized protein | 4.69 | 3 | 1 | 1 | 1 | 256 | 28.7 | 9.10 |
| | OS = Glycine max PE = 4 SV = 1 - | | | | | | | | |
| | [I1KXD2_SOYBN] | | | | | | | | |
| I1KXF2 | Uncharacterized protein | 34.94 | 7 | 3 | 3 | 3 | 166 | 18.4 | 6.52 |
| | OS = Glycine max PE = 4 SV = 1 - | | | | | | | | |
| | [I1KXF2_SOYBN] | | | | | | | | |
| I1KYK0 | Uncharacterized protein | 15.90 | 2 | 4 | 4 | 5 | 415 | 43.7 | 6.24 |
| | OS = Glycine max PE = 4 SV = 1 - | | | | | | | | |
| | [I1KYK0_SOYBN] | | | | | | | | |
| I1KYW3 | Uncharacterized protein | 41.87 | 2 | 8 | 8 | 30 | 203 | 22.6 | 7.24 |
| | OS = Glycine max PE = 4 SV = 1 - | | | | | | | | |
| | [I1KYW3_SOYBN] | | | | | | | | |
| I1KZ09 | Uncharacterized protein | 12.47 | 2 | 1 | 4 | 4 | 393 | 41.7 | 8.51 |
| | OS = Glycine max PE = 4 SV = 1 - | | | | | | | | |
| | [I1KZ09_SOYBN] | | | | | | | | |
| I1KZW8 | Uncharacterized protein | 2.24 | 1 | 1 | 1 | 1 | 312 | 33.6 | 5.31 |
| | OS = Glycine max PE = 4 SV = 1 - | | | | | | | | |
| | [I1KZW8_SOYBN] | | | | | | | | |
| I1L0E5 | Uncharacterized protein | 45.16 | 22 | 1 | 6 | 13 | 155 | 17.7 | 9.74 |
| | OS = Glycine max PE = 4 SV = 1 - | | | | | | | | |
| | [I1L0E5_SOYBN] | | | | | | | | |
| I1L0N6 | Uncharacterized protein | 2.24 | 1 | 1 | 1 | 1 | 313 | 33.9 | 7.33 |
| | OS = Glycine max PE = 4 SV = 1 - | | | | | | | | |
| | [I1L0N6_SOYBN] | | | | | | | | |
| I1L0Q8 | Uncharacterized protein | 14.85 | 6 | 5 | 5 | 5 | 458 | 49.0 | 6.84 |
| | OS = Glycine max PE = 4 SV = 1 - | | | | | | | | |
| | [I1L0Q8_SOYBN] | | | | | | | | |
| I1L0S5 | Uncharacterized protein | 23.20 | 1 | 3 | 4 | 4 | 181 | 19.0 | 6.93 |
| | OS = Glycine max PE = 4 SV = 1 - | | | | | | | | |
| | [I1L0S5_SOYBN] | | | | | | | | |
| I1L1D9 | Uncharacterized protein | 14.61 | 1 | 1 | 1 | 2 | 89 | 10.5 | 9.35 |
| | OS = Glycine max PE = 4 SV = 1 - | | | | | | | | |
| | [I1L1D9_SOYBN] | | | | | | | | |
| I1L1H3 | Uncharacterized protein | 4.47 | 4 | 1 | 1 | 1 | 492 | 54.7 | 6.64 |
| | OS = Glycine max PE = 4 SV = 1 - | | | | | | | | |
| | [I1L1H3_SOYBN] | | | | | | | | |
| I1L1U5 | Uncharacterized protein | 7.61 | 5 | 2 | 6 | 7 | 775 | 89.0 | 6.38 |
| | OS = Glycine max PE = 4 SV = 1 - | | | | | | | | |
| | [I1L1U5_SOYBN] | | | | | | | | |
| I1L2C2 | Uncharacterized protein | 1.06 | 4 | 1 | 1 | 1 | 660 | 74.9 | 6.18 |
| | OS = Glycine max PE = 4 SV = 1 - | | | | | | | | |
| | [I1L2C2_SOYBN] | | | | | | | | |
| I1L386 | Uncharacterized protein | 18.64 | 3 | 1 | 3 | 3 | 220 | 24.1 | 4.59 |
| | OS = Glycine max PE = 4 SV = 1 - | | | | | | | | |
| | [I1L386_SOYBN] | | | | | | | | |
| I1L3V7 | Uncharacterized protein | 3.43 | 2 | 3 | 3 | 3 | 729 | 81.9 | 9.11 |
| | OS = Glycine max PE = 4 SV = 1 - | | | | | | | | |
| | [I1L3V7_SOYBN] | | | | | | | | |
| I1L530 | Uncharacterized protein | 9.27 | 3 | 3 | 3 | 3 | 356 | 40.7 | 5.57 |
| | OS = Glycine max PE = 4 SV = 1 - | | | | | | | | |
| | [I1L530_SOYBN] | | | | | | | | |
| I1L5K6 | Uncharacterized protein | 6.65 | 2 | 3 | 3 | 3 | 496 | 55.6 | 6.71 |
| | OS = Glycine max PE = 4 SV = 1 - | | | | | | | | |
| | [I1L5K6_SOYBN] | | | | | | | | |
| I1L5Q0 | Uncharacterized protein | 11.08 | 4 | 4 | 4 | 5 | 352 | 40.9 | 9.61 |
| | OS = Glycine max PE = 4 SV = 1 - | | | | | | | | |
| | [I1L5Q0_SOYBN] | | | | | | | | |
| I1L6T4 | Uncharacterized protein | 12.26 | 2 | 4 | 4 | 4 | 416 | 42.5 | 5.97 |
| | OS = Glycine max PE = 4 SV = 1 - | | | | | | | | |
| | [I1L6T4_SOYBN] | | | | | | | | |
| I1L6W0 | Uncharacterized protein | 8.23 | 1 | 3 | 3 | 3 | 559 | 60.8 | 5.78 |
| | OS = Glycine max PE = 4 SV = 1 - | | | | | | | | |
| | [I1L6W0_SOYBN] | | | | | | | | |
| I1L700 | Uncharacterized protein | 7.01 | 5 | 4 | 4 | 4 | 585 | 64.7 | 5.43 |
| | OS = Glycine max PE = 4 SV = 1 - | | | | | | | | |
| | [I1L700_SOYBN] | | | | | | | | |
| I1L7F3 | Uncharacterized protein | 28.03 | 10 | 4 | 4 | 4 | 132 | 15.4 | 10.46 |
| | OS = Glycine max PE = 4 SV = 1 - | | | | | | | | |
| | [I1L7F3_SOYBN] | | | | | | | | |
| I1L849 | Uncharacterized protein | 70.61 | 1 | 12 | 12 | 18 | 262 | 27.4 | 5.29 |
| | OS = Glycine max PE = 4 SV = 1 - | | | | | | | | |
| | [I1L849_SOYBN] | | | | | | | | |
| I1L860 | Uncharacterized protein | 68.45 | 2 | 2 | 30 | 191 | 504 | 58.0 | 6.49 |
| | OS = Glycine max PE = 4 SV = 1 - | | | | | | | | |
| | [I1L860_SOYBN] | | | | | | | | |
| I1L8R2 | Uncharacterized protein | 43.40 | 6 | 3 | 13 | 16 | 371 | 40.9 | 10.73 |
| | OS = Glycine max PE = 4 SV = 1 - | | | | | | | | |
| | [I1L8R2_SOYBN] | | | | | | | | |
| I1L957 | Uncharacterized protein | 45.43 | 2 | 10 | 16 | 23 | 449 | 48.8 | 6.42 |
| | OS = Glycine max PE = 4 SV = 1 - | | | | | | | | |
| | [I1L957_SOYBN] | | | | | | | | |
| I1LAJ3 | Uncharacterized protein | 10.06 | 2 | 2 | 2 | 2 | 328 | 36.6 | 7.74 |
| | OS = Glycine max PE = 4 SV = 1 - | | | | | | | | |
| | [I1LAJ3_SOYBN] | | | | | | | | |
| I1LAW7 | Uncharacterized protein | 4.78 | 5 | 1 | 1 | 1 | 314 | 35.7 | 6.33 |
| | OS = Glycine max PE = 4 SV = 1 - | | | | | | | | |
| | [I1LAW7_SOYBN] | | | | | | | | |
| I1LC17 | Uncharacterized protein | 7.95 | 9 | 1 | 3 | 3 | 327 | 35.3 | 5.96 |
| | OS = Glycine max PE = 4 SV = 1 - | | | | | | | | |
| | [I1LC17_SOYBN] | | | | | | | | |
| I1LC49 | Uncharacterized protein | 3.15 | 1 | 1 | 1 | 2 | 444 | 47.3 | 8.24 |
| | OS = Glycine max PE = 4 SV = 1 - | | | | | | | | |
| | [I1LC49_SOYBN] | | | | | | | | |
| I1LCV0 | Uncharacterized protein | 25.19 | 3 | 1 | 1 | 1 | 131 | 14.1 | 9.79 |
| | OS = Glycine max PE = 4 SV = 1 - | | | | | | | | |
| | [I1LCV0_SOYBN] | | | | | | | | |
| I1LDC1 | Uncharacterized protein | 20.64 | 2 | 1 | 3 | 3 | 218 | 24.2 | 4.83 |
| | OS = Glycine max PE = 4 SV = 1 - | | | | | | | | |
| | [I1LDC1_SOYBN] | | | | | | | | |
| I1LDP2 | Uncharacterized protein | 11.24 | 4 | 3 | 3 | 3 | 249 | 27.9 | 8.92 |
| | OS = Glycine max PE = 4 SV = 1 - | | | | | | | | |
| | [I1LDP2_SOYBN] | | | | | | | | |
| I1LE33 | Uncharacterized protein | 35.62 | 3 | 17 | 20 | 38 | 584 | 68.2 | 6.32 |
| | OS = Glycine max PE = 4 SV = 1 - | | | | | | | | |
| | [I1LE33_SOYBN] | | | | | | | | |
| I1LE41 | Uncharacterized protein | 62.50 | 2 | 5 | 7 | 14 | 256 | 26.2 | 4.89 |
| | OS = Glycine max PE = 4 SV = 1 - | | | | | | | | |
| | [I1LE41_SOYBN] | | | | | | | | |
| I1LE85 | Uncharacterized protein | 2.40 | 3 | 1 | 1 | 1 | 334 | 37.4 | 9.48 |
| | OS = Glycine max PE = 4 SV = 1 - | | | | | | | | |
| | [I1LE85_SOYBN] | | | | | | | | |
| I1LFG4 | Uncharacterized protein | 7.95 | 1 | 4 | 4 | 4 | 742 | 80.2 | 6.57 |
| | OS = Glycine max PE = 4 SV = 1 - | | | | | | | | |
| | [I1LFG4_SOYBN] | | | | | | | | |
| I1LGD7 | Uncharacterized protein | 17.18 | 9 | 2 | 2 | 2 | 163 | 18.6 | 9.42 |
| | OS = Glycine max PE = 4 SV = 1 - | | | | | | | | |
| | [I1LGD7_SOYBN] | | | | | | | | |
| I1LH18 | Uncharacterized protein | 3.42 | 1 | 1 | 1 | 1 | 438 | 48.0 | 5.49 |
| | OS = Glycine max PE = 4 SV = 1 - | | | | | | | | |
| | [I1LH18_SOYBN] | | | | | | | | |
| I1LHH1 | Uncharacterized protein | 4.12 | 15 | 1 | 1 | 1 | 267 | 30.3 | 6.79 |
| | OS = Glycine max PE = 4 SV = 1 - | | | | | | | | |
| | [I1LHH1_SOYBN] | | | | | | | | |
| I1LHR9 | Uncharacterized protein | 35.96 | 3 | 2 | 2 | 2 | 114 | 12.6 | 7.34 |
| | OS = Glycine max PE = 4 SV = 1 - | | | | | | | | |
| | [I1LHR9_SOYBN] | | | | | | | | |
| I1LIJ6 | Uncharacterized protein | 4.37 | 2 | 1 | 3 | 3 | 687 | 77.2 | 9.16 |
| | OS = Glycine max PE = 4 SV = 1 - | | | | | | | | |
| | [I1LIJ6_SOYBN] | | | | | | | | |
| I1LIN2 | Uncharacterized protein | 1.84 | 1 | 2 | 2 | 2 | 1357 | 153.2 | 4.70 |
| | OS = Glycine max PE = 4 SV = 1 - | | | | | | | | |
| | [I1LIN2_SOYBN] | | | | | | | | |
| I1LIQ4 | Uncharacterized protein | 12.40 | 3 | 2 | 2 | 2 | 258 | 27.7 | 4.91 |
| | OS = Glycine max PE = 4 SV = 1 - | | | | | | | | |
| | [I1LIQ4_SOYBN] | | | | | | | | |
| I1LIT1 | Uncharacterized protein | 17.86 | 5 | 2 | 5 | 5 | 392 | 41.3 | 8.75 |
| | OS = Glycine max PE = 4 SV = 1 - | | | | | | | | |
| | [I1LIT1_SOYBN] | | | | | | | | |
| I1LIV8 | Uncharacterized protein | 30.89 | 3 | 2 | 8 | 14 | 259 | 29.4 | 10.20 |
| | OS = Glycine max PE = 4 SV = 1 - | | | | | | | | |
| | [I1LIV8_SOYBN] | | | | | | | | |
| I1LJE5 | Uncharacterized protein | 7.43 | 6 | 1 | 1 | 1 | 148 | 17.1 | 8.87 |
| | OS = Glycine max PE = 4 SV = 1 - | | | | | | | | |
| | [I1LJE5_SOYBN] | | | | | | | | |
| I1LJX3 | Uncharacterized protein | 23.13 | 3 | 1 | 4 | 4 | 147 | 16.2 | 5.99 |
| | OS = Glycine max PE = 4 SV = 1 - | | | | | | | | |
| | [I1LJX3_SOYBN] | | | | | | | | |
| I1LK37 | Uncharacterized protein | 18.95 | 1 | 1 | 3 | 3 | 248 | 26.4 | 7.61 |
| | OS = Glycine max PE = 4 SV = 1 - | | | | | | | | |
| | [I1LK37_SOYBN] | | | | | | | | |
| I1LM73 | Uncharacterized protein | 19.79 | 3 | 1 | 7 | 10 | 475 | 51.9 | 5.49 |
| | OS = Glycine max PE = 4 SV = 1 - | | | | | | | | |
| | [I1LM73_SOYBN] | | | | | | | | |
| I1LM95 | Uncharacterized protein | 7.96 | 1 | 1 | 1 | 2 | 226 | 24.6 | 9.74 |
| | OS = Glycine max PE = 4 SV = 1 - | | | | | | | | |
| | [I1LM95_SOYBN] | | | | | | | | |
| I1LMP2 | Uncharacterized protein | 3.88 | 2 | 1 | 1 | 1 | 309 | 34.8 | 6.28 |
| | OS = Glycine max PE = 4 SV = 1 - | | | | | | | | |
| | [I1LMP2_SOYBN] | | | | | | | | |
| I1LMV5 | Uncharacterized protein | 6.57 | 6 | 1 | 1 | 1 | 274 | 29.5 | 9.26 |
| | OS = Glycine max PE = 4 SV = 1 - | | | | | | | | |
| | [I1LMV5_SOYBN] | | | | | | | | |
| I1LN49 | Uncharacterized protein | 41.88 | 3 | 8 | 8 | 14 | 234 | 27.0 | 7.06 |
| | OS = Glycine max PE = 4 SV = 1 - | | | | | | | | |
| | [I1LN49_SOYBN] | | | | | | | | |
| I1LNK7 | Uncharacterized protein | 3.18 | 2 | 1 | 1 | 1 | 566 | 63.7 | 6.40 |
| | OS = Glycine max PE = 4 SV = 1 - | | | | | | | | |
| | [I1LNK7_SOYBN] | | | | | | | | |
| I1LP98 | Uncharacterized protein | 4.36 | 1 | 1 | 3 | 3 | 688 | 77.4 | 9.11 |
| | OS = Glycine max PE = 4 SV = 1 - | | | | | | | | |
| | [I1LP98_SOYBN] | | | | | | | | |
| I1LPN9 | Uncharacterized protein | 29.46 | 14 | 1 | 2 | 2 | 129 | 12.9 | 5.53 |
| | OS = Glycine max PE = 4 SV = 1 - | | | | | | | | |
| | [I1LPN9_SOYBN] | | | | | | | | |
| I1LQ03 | Uncharacterized protein | 3.13 | 3 | 1 | 1 | 1 | 415 | 45.8 | 8.18 |
| | OS = Glycine max PE = 4 SV = 1 - | | | | | | | | |
| | [I1LQ03_SOYBN] | | | | | | | | |
| I1LQR8 | Uncharacterized protein | 34.27 | 1 | 4 | 6 | 10 | 248 | 26.2 | 7.85 |
| | OS = Glycine max PE = 4 SV = 1 - | | | | | | | | |
| | [I1LQR8_SOYBN] | | | | | | | | |
| I1LRM2 | Uncharacterized protein | 2.88 | 7 | 1 | 1 | 1 | 313 | 34.3 | 8.46 |
| | OS = Glycine max PE = 4 SV = 1 - | | | | | | | | |
| | [I1LRM2_SOYBN] | | | | | | | | |
| I1LRP2 | Uncharacterized protein | 5.56 | 1 | 1 | 1 | 1 | 180 | 19.5 | 6.54 |
| | OS = Glycine max PE = 4 SV = 1 - | | | | | | | | |
| | [I1LRP2_SOYBN] | | | | | | | | |
| I1LS06 | Uncharacterized protein | 3.79 | 2 | 1 | 1 | 1 | 317 | 35.0 | 6.92 |
| | OS = Glycine max PE = 4 SV = 1 - | | | | | | | | |
| | [I1LS06_SOYBN] | | | | | | | | |
| I1LS33 | Uncharacterized protein | 3.57 | 7 | 1 | 1 | 1 | 308 | 34.6 | 5.21 |
| | OS = Glycine max PE = 4 SV = 1 - | | | | | | | | |
| | [I1LS33_SOYBN] | | | | | | | | |
| I1LS70 | Uncharacterized protein | 12.20 | 2 | 1 | 9 | 9 | 885 | 97.2 | 5.24 |
| | OS = Glycine max PE = 4 SV = 1 - | | | | | | | | |
| | [I1LS70_SOYBN] | | | | | | | | |
| I1LSB5 | Uncharacterized protein | 17.19 | 2 | 3 | 6 | 7 | 576 | 62.7 | 7.28 |
| | OS = Glycine max PE = 4 SV = 1 - | | | | | | | | |
| | [I1LSB5_SOYBN] | | | | | | | | |
| I1LSW3 | Uncharacterized protein | 6.37 | 4 | 3 | 3 | 3 | 455 | 50.1 | 5.73 |
| | OS = Glycine max PE = 4 SV = 1 - | | | | | | | | |
| | [I1LSW3_SOYBN] | | | | | | | | |
| I1LSY9 | Uncharacterized protein | 18.75 | 2 | 2 | 3 | 3 | 256 | 27.6 | 6.19 |
| | OS = Glycine max PE = 4 SV = 1 - | | | | | | | | |
| | [I1LSY9_SOYBN] | | | | | | | | |
| I1LT24 | Uncharacterized protein | 2.76 | 1 | 1 | 1 | 1 | 254 | 28.0 | 6.68 |
| | OS = Glycine max PE = 4 SV = 1 - | | | | | | | | |
| | [I1LT24_SOYBN] | | | | | | | | |
| I1LT76 | Uncharacterized protein | 14.88 | 7 | 3 | 3 | 3 | 242 | 26.6 | 6.86 |
| | OS = Glycine max PE = 4 SV = 1 - | | | | | | | | |
| | [I1LT76_SOYBN] | | | | | | | | |
| I1LTG9 | Uncharacterized protein | 29.52 | 4 | 2 | 2 | 2 | 105 | 10.6 | 11.72 |
| | OS = Glycine max PE = 4 SV = 1 - | | | | | | | | |
| | [I1LTG9_SOYBN] | | | | | | | | |
| I1LU39 | Uncharacterized protein | 17.71 | 6 | 2 | 2 | 3 | 175 | 18.6 | 5.02 |
| | OS = Glycine max PE = 4 SV = 1 - | | | | | | | | |
| | [I1LU39_SOYBN] | | | | | | | | |
| I1LV54 | Uncharacterized protein | 14.02 | 3 | 3 | 5 | 5 | 435 | 46.6 | 6.80 |
| | OS = Glycine max PE = 4 SV = 1 - | | | | | | | | |
| | [I1LV54_SOYBN] | | | | | | | | |
| I1LVH0 | Uncharacterized protein | 15.69 | 1 | 4 | 4 | 4 | 376 | 40.2 | 7.46 |
| | OS = Glycine max PE = 4 SV = 1 - | | | | | | | | |
| | [I1LVH0_SOYBN] | | | | | | | | |
| I1LWI3 | Uncharacterized protein | 15.73 | 14 | 4 | 4 | 4 | 286 | 31.5 | 5.24 |
| | OS = Glycine max PE = 4 SV = 1 - | | | | | | | | |
| | [I1LWI3_SOYBN] | | | | | | | | |
| I1LWV7 | Uncharacterized protein | 26.89 | 6 | 5 | 9 | 9 | 357 | 40.5 | 5.77 |
| | OS = Glycine max PE = 4 SV = 1 - | | | | | | | | |
| | [I1LWV7_SOYBN] | | | | | | | | |
| I1LX52 | Uncharacterized protein | 4.95 | 2 | 1 | 1 | 1 | 303 | 32.3 | 9.13 |
| | OS = Glycine max PE = 4 SV = 1 - | | | | | | | | |
| | [I1LX52_SOYBN] | | | | | | | | |
| I1LZ03 | Uncharacterized protein | 38.77 | 4 | 2 | 12 | 14 | 405 | 44.6 | 10.48 |
| | OS = Glycine max PE = 4 SV = 1 - | | | | | | | | |
| | [I1LZ03_SOYBN] | | | | | | | | |
| I1LZS4 | Uncharacterized protein | 0.95 | 3 | 1 | 1 | 1 | 943 | 104.3 | 6.23 |
| | OS = Glycine max PE = 4 SV = 1 - | | | | | | | | |
| | [I1LZS4_SOYBN] | | | | | | | | |
| I1M0K3 | Uncharacterized protein | 54.69 | 4 | 3 | 9 | 13 | 245 | 27.7 | 7.08 |
| | OS = Glycine max PE = 4 SV = 1 - | | | | | | | | |
| | [I1M0K3_SOYBN] | | | | | | | | |
| I1M0W1 | Uncharacterized protein | 48.92 | 2 | 3 | 3 | 5 | 139 | 15.1 | 5.86 |
| | OS = Glycine max PE = 4 SV = 1 - | | | | | | | | |
| | [I1M0W1_SOYBN] | | | | | | | | |
| I1M138 | Uncharacterized protein | 10.99 | 1 | 3 | 3 | 3 | 473 | 51.2 | 8.00 |
| | OS = Glycine max PE = 4 SV = 1 - | | | | | | | | |
| | [I1M138_SOYBN] | | | | | | | | |
| I1M214 | Uncharacterized protein | 23.58 | 7 | 2 | 2 | 2 | 106 | 11.6 | 9.41 |
| | OS = Glycine max PE = 4 SV = 1 - | | | | | | | | |
| | [I1M214_SOYBN] | | | | | | | | |
| I1M261 | Uncharacterized protein | 10.33 | 5 | 6 | 6 | 6 | 513 | 56.7 | 5.85 |
| | OS = Glycine max PE = 4 SV = 1 - | | | | | | | | |
| | [I1M261_SOYBN] | | | | | | | | |
| I1M292 | Uncharacterized protein | 6.22 | 1 | 1 | 1 | 1 | 241 | 25.7 | 8.43 |
| | OS = Glycine max PE = 4 SV = 1 - | | | | | | | | |
| | [I1M292_SOYBN] | | | | | | | | |
| I1M2S7 | Uncharacterized protein | 2.92 | 1 | 1 | 1 | 1 | 377 | 41.4 | 8.72 |
| | OS = Glycine max PE = 4 SV = 1 - | | | | | | | | |
| | [I1M2S7_SOYBN] | | | | | | | | |
| I1M2S9 | Uncharacterized protein | 3.63 | 2 | 1 | 1 | 1 | 303 | 34.9 | 9.32 |
| | OS = Glycine max PE = 4 SV = 1 - | | | | | | | | |
| | [I1M2S9_SOYBN] | | | | | | | | |
| I1M307 | Uncharacterized protein | 5.06 | 2 | 2 | 2 | 2 | 435 | 48.8 | 6.64 |
| | OS = Glycine max PE = 4 SV = 1 - | | | | | | | | |
| | [I1M307_SOYBN] | | | | | | | | |
| I1M3M9 | Uncharacterized protein | 37.48 | 4 | 16 | 16 | 29 | 643 | 67.9 | 6.58 |
| | OS = Glycine max PE = 4 SV = 1 - | | | | | | | | |
| | [I1M3M9_SOYBN] | | | | | | | | |
| I1M3Z4 | Uncharacterized protein | 10.89 | 1 | 1 | 2 | 2 | 248 | 26.8 | 5.21 |
| | OS = Glycine max PE = 4 SV = 1 - | | | | | | | | |
| | [I1M3Z4_SOYBN] | | | | | | | | |
| I1M4I4 | Uncharacterized protein | 11.11 | 1 | 1 | 1 | 1 | 216 | 22.6 | 5.14 |
| | OS = Glycine max PE = 4 SV = 1 - | | | | | | | | |
| | [I1M4I4_SOYBN] | | | | | | | | |
| I1M5E1 | Uncharacterized protein | 29.33 | 3 | 3 | 4 | 4 | 150 | 16.19 | 11.17 |
| | OS = Glycine max PE = 4 SV = 1 - | | | | | | | | |
| | [I1M5E1_SOYBN] | | | | | | | | |
| I1M5E2 | Uncharacterized protein | 6.40 | 1 | 1 | 1 | 1 | 203 | 22.6 | 7.20 |
| | OS = Glycine max PE = 4 SV = 1 - | | | | | | | | |
| | [I1M5E2_SOYBN] | | | | | | | | |
| I1M6M0 | Uncharacterized protein | 2.23 | 3 | 1 | 1 | 1 | 582 | 65.3 | 5.25 |
| | OS = Glycine max PE = 4 SV = 1 - | | | | | | | | |
| | [I1M6M0_SOYBN] | | | | | | | | |
| I1M6M4 | Uncharacterized protein | 2.57 | 1 | 1 | 1 | 1 | 545 | 61.2 | 5.21 |
| | OS = Glycine max PE = 4 SV = 1 - | | | | | | | | |
| | [I1M6M4_SOYBN] | | | | | | | | |
| I1M8L0 | Uncharacterized protein | 10.96 | 3 | 1 | 1 | 1 | 146 | 15.9 | 5.07 |
| | OS = Glycine max PE = 4 SV = 1 - | | | | | | | | |
| | [I1M8L0_SOYBN] | | | | | | | | |
| I1M984 | Uncharacterized protein | 12.89 | 2 | 6 | 6 | 6 | 543 | 60.2 | 6.10 |
| | OS = Glycine max PE = 4 SV = 1 - | | | | | | | | |
| | [I1M984_SOYBN] | | | | | | | | |
| I1M988 | Uncharacterized protein | 17.07 | 1 | 1 | 1 | 2 | 82 | 8.7 | 9.32 |
| | OS = Glycine max PE = 4 SV = 1 - | | | | | | | | |
| | [I1M988_SOYBN] | | | | | | | | |
| I1MAR9 | Uncharacterized protein | 12.24 | 2 | 8 | 8 | 8 | 776 | 85.0 | 7.34 |
| | OS = Glycine max PE = 4 SV = 1 - | | | | | | | | |
| | [I1MAR9_SOYBN] | | | | | | | | |
| I1MBI6 | Uncharacterized protein | 1.31 | 1 | 1 | 1 | 1 | 996 | 109.8 | 6.19 |
| | OS = Glycine max PE = 4 SV = 1 - | | | | | | | | |
| | [I1MBI6_SOYBN] | | | | | | | | |
| I1MBR7 | Uncharacterized protein | 36.46 | 1 | 5 | 13 | 17 | 469 | 51.4 | 5.34 |
| | OS = Glycine max PE = 4 SV = 1 - | | | | | | | | |
| | [I1MBR7_SOYBN] | | | | | | | | |
| I1MCF0 | Uncharacterized protein | 13.08 | 14 | 2 | 5 | 5 | 413 | 45.2 | 6.43 |
| | OS = Glycine max PE = 4 SV = 1 - | | | | | | | | |
| | [I1MCF0_SOYBN] | | | | | | | | |
| I1MDK0 | Uncharacterized protein | 2.94 | 5 | 1 | 1 | 1 | 306 | 32.8 | 6.25 |
| | OS = Glycine max PE = 4 SV = 1 - | | | | | | | | |
| | [I1MDK0_SOYBN] | | | | | | | | |
| I1MDY5 | Uncharacterized protein | 5.92 | 4 | 3 | 3 | 3 | 524 | 57.2 | 6.25 |
| | OS = Glycine max PE = 4 SV = 1 - | | | | | | | | |
| | [I1MDY5_SOYBN] | | | | | | | | |
| I1MEF8 | Uncharacterized protein | 2.48 | 1 | 1 | 1 | 1 | 322 | 35.3 | 5.19 |
| | OS = Glycine max PE = 4 SV = 1 - | | | | | | | | |
| | [I1MEF8_SOYBN] | | | | | | | | |
| I1MEX1 | Uncharacterized protein | 5.41 | 6 | 2 | 2 | 2 | 259 | 29.3 | 6.46 |
| | OS = Glycine max PE = 4 SV = 1 - | | | | | | | | |
| | [I1MEX1_SOYBN] | | | | | | | | |
| I1MGG5 | Uncharacterized protein | 37.04 | 2 | 2 | 3 | 4 | 135 | 14.6 | 7.14 |
| | OS = Glycine max PE = 4 SV = 1 - | | | | | | | | |
| | [I1MGG5_SOYBN] | | | | | | | | |
| I1MGG7 | Uncharacterized protein | 1.77 | 4 | 1 | 1 | 1 | 565 | 60.1 | 7.36 |
| | OS = Glycine max PE = 4 SV = 1 - | | | | | | | | |
| | [I1MGG7_SOYBN] | | | | | | | | |
| I1MGM0 | Uncharacterized protein | 16.77 | 6 | 2 | 2 | 2 | 167 | 19.0 | 4.73 |
| | OS = Glycine max PE = 4 SV = 1 - | | | | | | | | |
| | [I1MGM0_SOYBN] | | | | | | | | |
| I1MH07 | Uncharacterized protein | 4.57 | 8 | 2 | 2 | 2 | 634 | 70.7 | 5.64 |
| | OS = Glycine max PE = 4 SV = 1 - | | | | | | | | |
| | [I1MH07_SOYBN] | | | | | | | | |
| I1MJL6 | Uncharacterized protein | 46.12 | 17 | 11 | 11 | 11 | 232 | 25.9 | 10.15 |
| | OS = Glycine max PE = 4 SV = 1 - | | | | | | | | |
| | [I1MJL6_SOYBN] | | | | | | | | |
| I1MJN8 | Uncharacterized protein | 12.57 | 4 | 2 | 2 | 2 | 183 | 20.0 | 9.92 |
| | OS = Glycine max PE = 4 SV = 1 - | | | | | | | | |
| | [I1MJN8_SOYBN] | | | | | | | | |
| I1MK75 | Uncharacterized protein | 9.00 | 6 | 3 | 3 | 3 | 411 | 45.5 | 6.87 |
| | OS = Glycine max PE = 4 SV = 1 - | | | | | | | | |
| | [I1MK75_SOYBN] | | | | | | | | |
| I1MKY2 | Uncharacterized protein | 7.29 | 2 | 4 | 4 | 4 | 672 | 75.7 | 5.27 |
| | OS = Glycine max PE = 4 SV = 1 - | | | | | | | | |
| | [I1MKY2_SOYBN] | | | | | | | | |
| I1MKY3 | Uncharacterized protein | 39.29 | 9 | 3 | 4 | 5 | 112 | 12.6 | 6.74 |
| | OS = Glycine max PE = 4 SV = 1 - | | | | | | | | |
| | [I1MKY3_SOYBN] | | | | | | | | |
| I1MLW9 | Uncharacterized protein | 19.44 | 4 | 4 | 5 | 5 | 319 | 34.6 | 6.67 |
| | OS = Glycine max PE = 4 SV = 1 - | | | | | | | | |
| | [I1MLW9_SOYBN] | | | | | | | | |
| I1MM36 | Uncharacterized protein | 5.14 | 3 | 1 | 1 | 1 | 331 | 36.3 | 7.56 |
| | OS = Glycine max PE = 4 SV = 1 - | | | | | | | | |
| | [I1MM36_SOYBN] | | | | | | | | |
| I1MMH4 | Uncharacterized protein | 11.06 | 4 | 3 | 3 | 3 | 398 | 44.2 | 5.36 |
| | OS = Glycine max PE = 4 SV = 1 - | | | | | | | | |
| | [I1MMH4_SOYBN] | | | | | | | | |
| I1MMM3 | Uncharacterized protein | 6.31 | 1 | 1 | 1 | 1 | 206 | 23.3 | 7.11 |
| | OS = Glycine max PE = 4 SV = 1 - | | | | | | | | |
| | [I1MMM3_SOYBN] | | | | | | | | |
| I1MMY1 | Uncharacterized protein | 3.97 | 1 | 1 | 1 | 1 | 378 | 41.6 | 5.59 |
| | OS = Glycine max PE = 4 SV = 1 - | | | | | | | | |
| | [I1MMY1_SOYBN] | | | | | | | | |
| I1MN74 | Uncharacterized protein | 14.29 | 1 | 1 | 1 | 1 | 126 | 13.6 | 9.54 |
| | OS = Glycine max PE = 4 SV = 1 - | | | | | | | | |
| | [I1MN74_SOYBN] | | | | | | | | |
| I1MN82 | Uncharacterized protein | 4.10 | 2 | 2 | 2 | 2 | 488 | 55.3 | 5.87 |
| | OS = Glycine max PE = 4 SV = 1 - | | | | | | | | |
| | [I1MN82_SOYBN] | | | | | | | | |
| I1MR65 | Uncharacterized protein | 7.46 | 2 | 3 | 3 | 3 | 402 | 44.4 | 6.55 |
| | OS = Glycine max PE = 4 SV = 1 - | | | | | | | | |
| | [I1MR65_SOYBN] | | | | | | | | |
| I1MRV6 | Uncharacterized protein | 7.00 | 3 | 3 | 3 | 3 | 500 | 52.9 | 7.34 |
| | OS = Glycine max PE = 4 SV = 1 - | | | | | | | | |
| | [I1MRV6_SOYBN] | | | | | | | | |
| I1MT11 | Uncharacterized protein | 10.27 | 5 | 7 | 7 | 7 | 789 | 86.8 | 7.59 |
| | OS = Glycine max PE = 4 SV = 1 - | | | | | | | | |
| | [I1MT11_SOYBN] | | | | | | | | |
| I1MT57 | Uncharacterized protein | 1.54 | 6 | 1 | 1 | 1 | 454 | 49.9 | 6.49 |
| | OS = Glycine max PE = 4 SV = 1 - | | | | | | | | |
| | [I1MT57_SOYBN] | | | | | | | | |
| I1MTE2 | Uncharacterized protein | 6.88 | 1 | 1 | 1 | 1 | 160 | 17.3 | 9.47 |
| | OS = Glycine max PE = 4 SV = 1 - | | | | | | | | |
| | [I1MTE2_SOYBN] | | | | | | | | |
| I1MUH0 | Uncharacterized protein | 20.51 | 2 | 1 | 3 | 4 | 156 | 16.7 | 9.17 |
| | OS = Glycine max PE = 4 SV = 1 - | | | | | | | | |
| | [I1MUH0_SOYBN] | | | | | | | | |
| I1MX56 | Uncharacterized protein | 8.20 | 2 | 1 | 1 | 1 | 183 | 19.8 | 7.94 |
| | OS = Glycine max PE = 4 SV = 1 - | | | | | | | | |
| | [I1MX56_SOYBN] | | | | | | | | |
| I1MXZ6 | Uncharacterized protein | 67.48 | 3 | 7 | 7 | 9 | 123 | 13.4 | 4.88 |
| | OS = Glycine max PE = 4 SV = 1 - | | | | | | | | |
| | [I1MXZ6_SOYBN] | | | | | | | | |
| I1N0P0 | Uncharacterized protein | 13.39 | 7 | 2 | 2 | 2 | 112 | 12.8 | 9.03 |
| | OS = Glycine max PE = 4 SV = 1 - | | | | | | | | |
| | [I1N0P0_SOYBN] | | | | | | | | |
| I1N1W7 | Uncharacterized protein | 8.54 | 5 | 5 | 5 | 7 | 527 | 58.8 | 6.99 |
| | OS = Glycine max PE = 4 SV = 1 - | | | | | | | | |
| | [I1N1W7_SOYBN] | | | | | | | | |
| I1N1Y7 | Uncharacterized protein | 87.78 | 2 | 4 | 4 | 4 | 90 | 8.5 | 6.95 |
| | OS = Glycine max PE = 4 SV = 1 - | | | | | | | | |
| | [I1N1Y7_SOYBN] | | | | | | | | |
| I1N2I4 | Uncharacterized protein | 18.43 | 1 | 1 | 4 | 5 | 255 | 27.6 | 6.04 |
| | OS = Glycine max PE = 4 SV = 1 - | | | | | | | | |
| | [I1N2I4_SOYBN] | | | | | | | | |
| I1N466 | Uncharacterized protein | 21.08 | 2 | 5 | 5 | 5 | 370 | 39.9 | 6.30 |
| | OS = Glycine max PE = 4 SV = 1 - | | | | | | | | |
| | [I1N466_SOYBN] | | | | | | | | |
| I1N4C1 | Uncharacterized protein | 6.26 | 1 | 3 | 3 | 3 | 511 | 56.7 | 6.04 |
| | OS = Glycine max PE = 4 SV = 1 - | | | | | | | | |
| | [I1N4C1_SOYBN] | | | | | | | | |
| I1N5R7 | Uncharacterized protein | 23.29 | 7 | 5 | 6 | 6 | 322 | 34.7 | 6.25 |
| | OS = Glycine max PE = 4 SV = 1 - | | | | | | | | |
| | [I1N5R7_SOYBN] | | | | | | | | |
| I1N628 | Uncharacterized protein | 17.03 | 3 | 3 | 5 | 5 | 276 | 29.7 | 9.07 |
| | OS = Glycine max PE = 4 SV = 1 - | | | | | | | | |
| | [I1N628_SOYBN] | | | | | | | | |
| I1N7P5 | Uncharacterized protein | 11.24 | 6 | 1 | 1 | 1 | 169 | 18.5 | 5.47 |
| | OS = Glycine max PE = 4 SV = 1 - | | | | | | | | |
| | [I1N7P5_SOYBN] | | | | | | | | |
| I1N7Z4 | Uncharacterized protein | 4.24 | 1 | 1 | 1 | 1 | 377 | 42.8 | 6.09 |
| | OS = Glycine max PE = 4 SV = 1 - | | | | | | | | |
| | [I1N7Z4_SOYBN] | | | | | | | | |
| I1NAK9 | Uncharacterized protein | 6.11 | 2 | 1 | 5 | 6 | 868 | 98.8 | 5.64 |
| | OS = Glycine max PE = 4 SV = 1 - | | | | | | | | |
| | [I1NAK9_SOYBN] | | | | | | | | |
| I1NBD9 | Uncharacterized protein | 2.91 | 2 | 2 | 2 | 2 | 860 | 95.2 | 6.02 |
| | OS = Glycine max PE = 4 SV = 1 - | | | | | | | | |
| | [I1NBD9_SOYBN] | | | | | | | | |
| I1NC19 | Uncharacterized protein | 3.67 | 5 | 1 | 1 | 1 | 436 | 48.8 | 8.32 |
| | OS = Glycine max PE = 4 SV = 1 - | | | | | | | | |
| | [I1NC19_SOYBN] | | | | | | | | |
| I1NC66 | Uncharacterized protein | 3.70 | 5 | 1 | 1 | 1 | 216 | 24.7 | 7.55 |
| | OS = Glycine max PE = 4 SV = 1 - | | | | | | | | |
| | [I1NC66_SOYBN] | | | | | | | | |
| I1ND48 | Uncharacterized protein | 10.89 | 1 | 1 | 1 | 1 | 101 | 11.6 | 7.65 |
| | OS = Glycine max PE = 4 SV = 1 - | | | | | | | | |
| | [I1ND48_SOYBN] | | | | | | | | |
| I1NFD0 | Uncharacterized protein | 3.95 | 7 | 1 | 1 | 1 | 228 | 25.3 | 4.83 |
| | OS = Glycine max PE = 4 SV = 1 - | | | | | | | | |
| | [I1NFD0_SOYBN] | | | | | | | | |
| I1NGS1 | Uncharacterized protein | 37.33 | 4 | 3 | 5 | 5 | 225 | 24.9 | 4.61 |
| | OS = Glycine max PE = 4 SV = 1 - | | | | | | | | |
| | [I1NGS1_SOYBN] | | | | | | | | |
| I1NH59 | Uncharacterized protein | 20.18 | 1 | 1 | 3 | 3 | 223 | 24.7 | 4.86 |
| | OS = Glycine max PE = 4 SV = 1 - | | | | | | | | |
| | [I1NH59_SOYBN] | | | | | | | | |
| I1NJ30 | Uncharacterized protein | 31.55 | 1 | 1 | 5 | 8 | 187 | 21.0 | 11.17 |
| | OS = Glycine max PE = 4 SV = 1 - | | | | | | | | |
| | [I1NJ30_SOYBN] | | | | | | | | |
| I1NJ48 | Uncharacterized protein | 5.33 | 13 | 2 | 2 | 2 | 413 | 45.9 | 7.50 |
| | OS = Glycine max PE = 4 SV = 1 - | | | | | | | | |
| | [I1NJ48_SOYBN] | | | | | | | | |
| I1NJ89 | Uncharacterized protein | 1.97 | 1 | 1 | 1 | 1 | 610 | 68.5 | 5.81 |
| | OS = Glycine max PE = 4 SV = 1 - | | | | | | | | |
| | [I1NJ89_SOYBN] | | | | | | | | |
| I1NJC3 | Uncharacterized protein | 3.13 | 2 | 1 | 1 | 1 | 447 | 50.3 | 6.21 |
| | OS = Glycine max PE = 4 SV = 1 - | | | | | | | | |
| | [I1NJC3_SOYBN] | | | | | | | | |
| K7K0R1 | Uncharacterized protein | 2.22 | 2 | 1 | 1 | 1 | 450 | 49.3 | 7.01 |
| | OS = Glycine max PE = 4 SV = 1 - | | | | | | | | |
| | [K7K0R1_SOYBN] | | | | | | | | |
| K7K185 | Uncharacterized protein | 4.04 | 4 | 1 | 1 | 1 | 223 | 23.9 | 7.97 |
| | OS = Glycine max PE = 4 SV = 1 - | | | | | | | | |
| | [K7K185_SOYBN] | | | | | | | | |
| K7K1R2 | Uncharacterized protein | 10.84 | 2 | 4 | 4 | 4 | 443 | 47.8 | 5.83 |
| | OS = Glycine max PE = 4 SV = 1 - | | | | | | | | |
| | [K7K1R2_SOYBN] | | | | | | | | |
| K7K480 | Uncharacterized protein | 18.31 | 4 | 1 | 1 | 1 | 71 | 8.3 | 6.65 |
| | OS = Glycine max PE = 4 SV = 1 - | | | | | | | | |
| | [K7K480_SOYBN] | | | | | | | | |
| K7K4G2 | Uncharacterized protein | 31.92 | 4 | 13 | 21 | 32 | 614 | 71.7 | 5.49 |
| | OS = Glycine max PE = 4 SV = 1 - | | | | | | | | |
| | [K7K4G2_SOYBN] | | | | | | | | |
| K7KAG8 | Uncharacterized protein | 26.04 | 2 | 2 | 11 | 15 | 503 | 55.3 | 5.33 |
| | OS = Glycine max PE = 4 SV = 1 - | | | | | | | | |
| | [K7KAG8_SOYBN] | | | | | | | | |
| K7KHJ7 | Uncharacterized protein | 14.05 | 1 | 1 | 2 | 2 | 185 | 18.0 | 8.12 |
| | OS = Glycine max PE = 4 SV = 1 - | | | | | | | | |
| | [K7KHJ7_SOYBN] | | | | | | | | |
| K7KI86 | Uncharacterized protein | 5.38 | 4 | 4 | 4 | 5 | 873 | 98.5 | 5.64 |
| | OS = Glycine max PE = 4 SV = 1 - | | | | | | | | |
| | [K7KI86_SOYBN] | | | | | | | | |
| K7KJZ4 | Uncharacterized protein | 21.37 | 2 | 1 | 2 | 2 | 117 | 13.1 | 6.52 |
| | OS = Glycine max PE = 4 SV = 1 - | | | | | | | | |
| | [K7KJZ4_SOYBN] | | | | | | | | |
| K7KNG2 | Uncharacterized protein | 4.92 | 3 | 2 | 2 | 2 | 630 | 70.7 | 5.58 |
| | OS = Glycine max PE = 4 SV = 1 - | | | | | | | | |
| | [K7KNG2_SOYBN] | | | | | | | | |
| K7KPK0 | Uncharacterized protein | 2.89 | 3 | 1 | 1 | 1 | 346 | 38.8 | 6.04 |
| | OS = Glycine max PE = 4 SV = 1 - | | | | | | | | |
| | [K7KPK0_SOYBN] | | | | | | | | |
| K7KQ08 | Uncharacterized protein | 37.31 | 1 | 1 | 1 | 1 | 67 | 7.5 | 9.09 |
| | OS = Glycine max PE = 4 SV = 1 - | | | | | | | | |
| | [K7KQ08_SOYBN] | | | | | | | | |
| K7KQ65 | Uncharacterized protein | 3.40 | 7 | 1 | 1 | 1 | 294 | 31.8 | 9.28 |
| | OS = Glycine max PE = 4 SV = 1 - | | | | | | | | |
| | [K7KQ65_SOYBN] | | | | | | | | |
| K7KR01 | Uncharacterized protein | 2.03 | 4 | 1 | 1 | 1 | 640 | 70.7 | 5.21 |
| | OS = Glycine max PE = 4 SV = 1 - | | | | | | | | |
| | [K7KR01_SOYBN] | | | | | | | | |
| K7KRL8 | Uncharacterized protein | 5.80 | 2 | 1 | 1 | 1 | 293 | 31.8 | 5.86 |
| | OS = Glycine max PE = 4 SV = 1 - | | | | | | | | |
| | [K7KRL8_SOYBN] | | | | | | | | |
| K7KVU4 | Uncharacterized protein | 9.71 | 5 | 1 | 1 | 1 | 103 | 11.4 | 5.91 |
| | OS = Glycine max PE = 4 SV = 1 - | | | | | | | | |
| | [K7KVU4_SOYBN] | | | | | | | | |
| K7KW93 | Uncharacterized protein | 0.77 | 2 | 1 | 1 | 1 | 1037 | 116.0 | 7.27 |
| | OS = Glycine max PE = 4 SV = 1 - | | | | | | | | |
| | [K7KW93_SOYBN] | | | | | | | | |
| K7KWC6 | Uncharacterized protein | 4.71 | 1 | 1 | 1 | 1 | 191 | 20.7 | 10.65 |
| | OS = Glycine max PE = 4 SV = 1 - | | | | | | | | |
| | [K7KWC6_SOYBN] | | | | | | | | |
| K7KXX0 | Uncharacterized protein | 12.78 | 1 | 10 | 10 | 10 | 1056 | 116.4 | 8.70 |
| | OS = Glycine max PE = 4 SV = 1 - | | | | | | | | |
| | [K7KXX0_SOYBN] | | | | | | | | |
| K7KZU4 | Uncharacterized protein | 2.18 | 2 | 2 | 2 | 2 | 962 | 107.7 | 5.26 |
| | OS = Glycine max PE = 4 SV = 1 - | | | | | | | | |
| | [K7KZU4_SOYBN] | | | | | | | | |
| K7KZX8 | Uncharacterized protein | 32.07 | 15 | 6 | 6 | 7 | 184 | 20.8 | 6.10 |
| | OS = Glycine max PE = 4 SV = 1 - | | | | | | | | |
| | [K7KZX8_SOYBN] | | | | | | | | |
| K7L362 | Uncharacterized protein | 7.63 | 5 | 1 | 1 | 1 | 118 | 12.6 | 5.40 |
| | OS = Glycine max PE = 4 SV = 1 - | | | | | | | | |
| | [K7L362_SOYBN] | | | | | | | | |
| K7L4F4 | Uncharacterized protein | 7.05 | 1 | 1 | 6 | 7 | 922 | 104.1 | 6.04 |
| | OS = Glycine max PE = 4 SV = 1 - | | | | | | | | |
| | [K7L4F4_SOYBN] | | | | | | | | |
| K7L5X9 | Uncharacterized protein | 13.33 | 2 | 1 | 1 | 1 | 90 | 9.8 | 9.70 |
| | OS = Glycine max PE = 4 SV = 1 - | | | | | | | | |
| | [K7L5X9_SOYBN] | | | | | | | | |
| K7L5Y2 | Uncharacterized protein | 7.77 | 2 | 1 | 1 | 1 | 103 | 11.7 | 8.69 |
| | OS = Glycine max PE = 4 SV = 1 - | | | | | | | | |
| | [K7L5Y2_SOYBN] | | | | | | | | |
| K7L7E6 | Uncharacterized protein | 7.00 | 7 | 1 | 2 | 2 | 200 | 22.6 | 8.47 |
| | OS = Glycine max PE = 4 SV = 1 - | | | | | | | | |
| | [K7L7E6_SOYBN] | | | | | | | | |
| K7L7I8 | Uncharacterized protein | 1.35 | 2 | 1 | 1 | 1 | 1036 | 117.9 | 6.14 |
| | OS = Glycine max PE = 4 SV = 1 - | | | | | | | | |
| | [K7L7I8_SOYBN] | | | | | | | | |
| K7L8B5 | Uncharacterized protein | 61.31 | 2 | 7 | 7 | 12 | 137 | 15.3 | 4.64 |
| | OS = Glycine max PE = 4 SV = 1 - | | | | | | | | |
| | [K7L8B5_SOYBN] | | | | | | | | |
| K7L8B8 | Uncharacterized protein | 10.53 | 2 | 1 | 1 | 1 | 76 | 8.7 | 10.11 |
| | OS = Glycine max PE = 4 SV = 1 - | | | | | | | | |
| | [K7L8B8_SOYBN] | | | | | | | | |
| K7L8K5 | Uncharacterized protein | 12.20 | 2 | 2 | 3 | 3 | 451 | 48.8 | 8.16 |
| | OS = Glycine max PE = 4 SV = 1 - | | | | | | | | |
| | [K7L8K5_SOYBN] | | | | | | | | |
| K7L9E4 | Uncharacterized protein | 7.35 | 1 | 1 | 1 | 1 | 204 | 23.1 | 4.97 |
| | OS = Glycine max PE = 4 SV = 1 - | | | | | | | | |
| | [K7L9E4_SOYBN] | | | | | | | | |
| K7LA98 | Uncharacterized protein | 1.53 | 7 | 1 | 1 | 1 | 653 | 71.5 | 6.89 |
| | OS = Glycine max PE = 4 SV = 1 - | | | | | | | | |
| | [K7LA98_SOYBN] | | | | | | | | |
| K7LBQ0 | Uncharacterized protein | 2.52 | 4 | 1 | 1 | 1 | 595 | 64.2 | 7.23 |
| | OS = Glycine max PE = 4 SV = 1 - | | | | | | | | |
| | [K7LBQ0_SOYBN] | | | | | | | | |
| K7LCD1 | Uncharacterized protein | 1.95 | 3 | 1 | 1 | 1 | 512 | 56.8 | 5.01 |
| | OS = Glycine max PE = 4 SV = 1 - | | | | | | | | |
| | [K7LCD1_SOYBN] | | | | | | | | |
| K7LDT9 | Uncharacterized protein | 14.00 | 2 | 9 | 9 | 11 | 950 | 103.3 | 6.76 |
| | OS = Glycine max PE = 4 SV = 1 - | | | | | | | | |
| | [K7LDT9_SOYBN] | | | | | | | | |
| K7LIN7 | Uncharacterized protein | 28.35 | 2 | 3 | 3 | 3 | 127 | 14.3 | 4.08 |
| | OS = Glycine max PE = 4 SV = 1 - | | | | | | | | |
| | [K7LIN7_SOYBN] | | | | | | | | |
| K7LKA7 | Uncharacterized protein | 2.12 | 2 | 1 | 1 | 1 | 471 | 47.5 | 7.55 |
| | OS = Glycine max PE = 4 SV = 1 - | | | | | | | | |
| | [K7LKA7_SOYBN] | | | | | | | | |
| K7LLW8 | Uncharacterized protein | 1.29 | 2 | 1 | 1 | 1 | 1085 | 117.2 | 7.88 |
| | OS = Glycine max PE = 4 SV = 1 - | | | | | | | | |
| | [K7LLW8_SOYBN] | | | | | | | | |
| K7LM15 | Uncharacterized protein | 1.46 | 3 | 1 | 1 | 1 | 616 | 69.7 | 5.64 |
| | OS = Glycine max PE = 4 SV = 1 - | | | | | | | | |
| | [K7LM15_SOYBN] | | | | | | | | |
| K7LP83 | Uncharacterized protein | 1.98 | 5 | 1 | 1 | 2 | 505 | 55.5 | 5.49 |
| | OS = Glycine max PE = 4 SV = 1 - | | | | | | | | |
| | [K7LP83_SOYBN] | | | | | | | | |
| K7LP91 | Uncharacterized protein | 1.70 | 1 | 1 | 1 | 1 | 763 | 83.6 | 8.15 |
| | OS = Glycine max PE = 4 SV = 1 - | | | | | | | | |
| | [K7LP91_SOYBN] | | | | | | | | |
| K7LP96 | Uncharacterized protein | 38.60 | 8 | 3 | 4 | 5 | 171 | 16.6 | 5.63 |
| | OS = Glycine max PE = 4 SV = 1 - | | | | | | | | |
| | [K7LP96_SOYBN] | | | | | | | | |
| K7LQ70 | Uncharacterized protein | 26.99 | 3 | 6 | 16 | 19 | 793 | 88.9 | 5.38 |
| | OS = Glycine max PE = 4 SV = 1 - | | | | | | | | |
| | [K7LQ70_SOYBN] | | | | | | | | |
| K7LQI7 | Uncharacterized protein | 10.92 | 1 | 1 | 1 | 1 | 119 | 13.4 | 5.58 |
| | OS = Glycine max PE = 4 SV = 1 - | | | | | | | | |
| | [K7LQI7_SOYBN] | | | | | | | | |
| K7LSD2 | Uncharacterized protein | 7.60 | 3 | 5 | 5 | 5 | 934 | 102.5 | 5.29 |
| | OS = Glycine max PE = 4 SV = 1 - | | | | | | | | |
| | [K7LSD2_SOYBN] | | | | | | | | |
| K7LSI7 | Uncharacterized protein | 5.62 | 12 | 3 | 3 | 3 | 729 | 82.4 | 6.61 |
| | OS = Glycine max PE = 4 SV = 1 - | | | | | | | | |
| | [K7LSI7_SOYBN] | | | | | | | | |
| K7LUU3 | Uncharacterized protein | 15.00 | 3 | 1 | 2 | 3 | 160 | 17.2 | 4.18 |
| | OS = Glycine max PE = 4 SV = 1 - | | | | | | | | |
| | [K7LUU3_SOYBN] | | | | | | | | |
| K7LWC6 | Uncharacterized protein | 0.60 | 3 | 1 | 1 | 1 | 1006 | 113.8 | 6.55 |
| | OS = Glycine max PE = 4 SV = 1 - | | | | | | | | |
| | [K7LWC6_SOYBN] | | | | | | | | |
| K7M206 | Uncharacterized protein | 0.98 | 1 | 1 | 1 | 1 | 713 | 79.5 | 7.12 |
| | OS = Glycine max PE = 4 SV = 1 - | | | | | | | | |
| | [K7M206_SOYBN] | | | | | | | | |
| K7M3Y6 | Uncharacterized protein | 17.03 | 14 | 3 | 6 | 7 | 417 | 45.9 | 6.58 |
| | OS = Glycine max PE = 4 SV = 1 - | | | | | | | | |
| | [K7M3Y6_SOYBN] | | | | | | | | |
| K7M572 | Uncharacterized protein | 4.58 | 3 | 1 | 1 | 1 | 284 | 31.7 | 6.74 |
| | OS = Glycine max PE = 4 SV = 1 - | | | | | | | | |
| | [K7M572_SOYBN] | | | | | | | | |
| K7M6Q3 | Uncharacterized protein | 2.44 | 6 | 1 | 1 | 1 | 287 | 32.0 | 8.94 |
| | OS = Glycine max PE = 4 SV = 1 - | | | | | | | | |
| | [K7M6Q3_SOYBN] | | | | | | | | |
| K7M6V7 | Uncharacterized protein | 1.39 | 4 | 1 | 1 | 1 | 647 | 70.9 | 6.28 |
| | OS = Glycine max PE = 4 SV = 1 - | | | | | | | | |
| | [K7M6V7_SOYBN] | | | | | | | | |
| K7M9C0 | Uncharacterized protein | 0.88 | 2 | 1 | 1 | 1 | 1252 | 138.2 | 7.14 |
| | OS = Glycine max PE = 4 SV = 1 - | | | | | | | | |
| | [K7M9C0_SOYBN] | | | | | | | | |
| K7MB76 | Uncharacterized protein | 5.43 | 4 | 1 | 1 | 1 | 184 | 20.6 | 5.30 |
| | OS = Glycine max PE = 4 SV = 1 - | | | | | | | | |
| | [K7MB76_SOYBN] | | | | | | | | |
| K7MFW9 | Uncharacterized protein | 1.57 | 4 | 1 | 1 | 1 | 765 | 84.6 | 5.95 |
| | OS = Glycine max PE = 4 SV = 1 - | | | | | | | | |
| | [K7MFW9_SOYBN] | | | | | | | | |
| K7MIA6 | Uncharacterized protein | 16.82 | 7 | 1 | 3 | 3 | 214 | 24.0 | 9.06 |
| | OS = Glycine max PE = 4 SV = 1 - | | | | | | | | |
| | [K7MIA6_SOYBN] | | | | | | | | |
| K7MID0 | Uncharacterized protein | 3.60 | 3 | 1 | 3 | 3 | 889 | 96.9 | 6.39 |
| | OS = Glycine max PE = 4 SV = 1 - | | | | | | | | |
| | [K7MID0_SOYBN] | | | | | | | | |
| K7MIP9 | Uncharacterized protein | 3.76 | 5 | 1 | 1 | 2 | 186 | 20.6 | 4.70 |
| | OS = Glycine max PE = 4 SV = 1 - | | | | | | | | |
| | [K7MIP9_SOYBN] | | | | | | | | |
| K7MJY8 | Uncharacterized protein | 26.34 | 3 | 14 | 19 | 26 | 805 | 92.1 | 6.35 |
| | OS = Glycine max PE = 4 SV = 1 - | | | | | | | | |
| | [K7MJY8_SOYBN] | | | | | | | | |
| K7MJZ7 | Uncharacterized protein | 5.18 | 2 | 1 | 1 | 1 | 193 | 21.4 | 4.42 |
| | OS = Glycine max PE = 4 SV = 1 - | | | | | | | | |
| | [K7MJZ7_SOYBN] | | | | | | | | |
| K7MM36 | Uncharacterized protein | 13.04 | 5 | 1 | 1 | 1 | 92 | 10.4 | 7.52 |
| | OS = Glycine max PE = 4 SV = 1 - | | | | | | | | |
| | [K7MM36_SOYBN] | | | | | | | | |
| K7MP05 | Uncharacterized protein | 1.56 | 1 | 1 | 1 | 1 | 959 | 108.0 | 8.72 |
| | OS = Glycine max PE = 4 SV = 1 - | | | | | | | | |
| | [K7MP05_SOYBN] | | | | | | | | |
| K7MPL7 | Uncharacterized protein | 4.20 | 1 | 1 | 1 | 1 | 333 | 36.8 | 9.11 |
| | OS = Glycine max PE = 4 SV = 1 - | | | | | | | | |
| | [K7MPL7_SOYBN] | | | | | | | | |
| K7MQ64 | Uncharacterized protein | 5.76 | 8 | 1 | 1 | 1 | 139 | 15.3 | 6.54 |
| | OS = Glycine max PE = 4 SV = 1 - | | | | | | | | |
| | [K7MQ64_SOYBN] | | | | | | | | |
| K7MQS5 | Uncharacterized protein | 3.73 | 2 | 1 | 1 | 1 | 295 | 32.6 | 8.79 |
| | OS = Glycine max PE = 4 SV = 1 - | | | | | | | | |
| | [K7MQS5_SOYBN] | | | | | | | | |
| K7MSK8 | Uncharacterized protein | 23.40 | 2 | 1 | 1 | 1 | 47 | 5.4 | 9.47 |
| | OS = Glycine max PE = 4 SV = 1 - | | | | | | | | |
| | [K7MSK8_SOYBN] | | | | | | | | |
| K7MUH0 | Uncharacterized protein | 7.69 | 5 | 2 | 2 | 2 | 221 | 24.7 | 6.44 |
| | OS = Glycine max PE = 4 SV = 1 - | | | | | | | | |
| | [K7MUH0_SOYBN] | | | | | | | | |
| K7MVB9 | Uncharacterized protein | 9.37 | 2 | 6 | 6 | 6 | 1025 | 116.0 | 6.86 |
| | OS = Glycine max PE = 4 SV = 1 - | | | | | | | | |
| | [K7MVB9_SOYBN] | | | | | | | | |
| K7MVL0 | Uncharacterized protein | 27.95 | 6 | 4 | 4 | 4 | 161 | 17.3 | 5.35 |
| | OS = Glycine max PE = 4 SV = 1 - | | | | | | | | |
| | [K7MVL0_SOYBN] | | | | | | | | |
| K7MX62 | Uncharacterized protein | 42.58 | 2 | 2 | 4 | 10 | 155 | 17.0 | 9.35 |
| | OS = Glycine max PE = 4 SV = 1 - | | | | | | | | |
| | [K7MX62_SOYBN] | | | | | | | | |
| K7N0P4 | Uncharacterized protein | 12.10 | 1 | 1 | 1 | 1 | 124 | 12.2 | 7.40 |
| | OS = Glycine max PE = 4 SV = 1 - | | | | | | | | |
| | [K7N0P4_SOYBN] | | | | | | | | |
| K7N277 | Uncharacterized protein | 35.53 | 1 | 2 | 2 | 2 | 152 | 13.9 | 10.87 |
| | OS = Glycine max PE = 4 SV = 1 - | | | | | | | | |
| | [K7N277_SOYBN] | | | | | | | | |
| K7N2N0 | Uncharacterized protein | 26.60 | 39 | 3 | 3 | 3 | 94 | 10.4 | 8.69 |
| | OS = Glycine max PE = 4 SV = 1 - | | | | | | | | |
| | [K7N2N0_SOYBN] | | | | | | | | |
| K7N388 | Uncharacterized protein | 31.48 | 3 | 2 | 2 | 3 | 54 | 6.1 | 9.77 |
| | OS = Glycine max PE = 4 SV = 1 - | | | | | | | | |
| | [K7N388_SOYBN] | | | | | | | | |
| K7N4I7 | Uncharacterized protein | 15.69 | 1 | 2 | 2 | 7 | 204 | 22.1 | 9.28 |
| | OS = Glycine max PE = 4 SV = 1 - | | | | | | | | |
| | [K7N4I7_SOYBN] | | | | | | | | |
| I1J601 | Uncharacterized protein | 18.10 | 2 | 1 | 1 | 1 | 116 | 12.7 | 9.89 |
| | OS = Glycine max PE = 4 SV = 2 - | | | | | | | | |
| | [I1J601_SOYBN] | | | | | | | | |
| I1JAY8 | Uncharacterized protein | 11.35 | 4 | 4 | 4 | 4 | 643 | 71.2 | 5.77 |
| | OS = Glycine max PE = 4 SV = 2 - | | | | | | | | |
| | [I1JAY8_SOYBN] | | | | | | | | |
| I1JEI0 | Uncharacterized protein | 19.57 | 7 | 1 | 7 | 9 | 368 | 41.5 | 5.97 |
| | OS = Glycine max PE = 4 SV = 2 - | | | | | | | | |
| | [I1JEI0_SOYBN] | | | | | | | | |
| I1JF86 | Uncharacterized protein | 65.81 | 4 | 17 | 28 | 95 | 506 | 57.8 | 6.60 |
| | OS = Glycine max PE = 4 SV = 2 - | | | | | | | | |
| | [I1JF86_SOYBN] | | | | | | | | |
| I1JG95 | Uncharacterized protein | 0.97 | 4 | 1 | 1 | 1 | 930 | 102.6 | 5.92 |
| | OS = Glycine max PE = 4 SV = 2 - | | | | | | | | |
| | [I1JG95_SOYBN] | | | | | | | | |
| I1JLL2 | Uncharacterized protein | 8.72 | 2 | 1 | 1 | 1 | 195 | 22.0 | 6.95 |
| | OS = Glycine max PE = 4 SV = 2 - | | | | | | | | |
| | [I1JLL2_SOYBN] | | | | | | | | |
| I1JPL2 | Uncharacterized protein | 2.45 | 2 | 1 | 1 | 1 | 490 | 54.2 | 5.99 |
| | OS = Glycine max PE = 4 SV = 2 - | | | | | | | | |
| | [I1JPL2_SOYBN] | | | | | | | | |
| I1JSX0 | Uncharacterized protein | 44.55 | 1 | 1 | 3 | 4 | 101 | 11.0 | 5.22 |
| | OS = Glycine max PE = 4 SV = 2 - | | | | | | | | |
| | [I1JSX0_SOYBN] | | | | | | | | |
| I1K3X4 | Uncharacterized protein | 2.45 | 3 | 2 | 2 | 2 | 981 | 109.4 | 4.86 |
| | OS = Glycine max PE = 4 SV = 2 - | | | | | | | | |
| | [I1K3X4_SOYBN] | | | | | | | | |
| I1K689 | Uncharacterized protein | 1.23 | 5 | 1 | 1 | 1 | 568 | 63.7 | 6.54 |
| | OS = Glycine max PE = 4 SV = 2 - | | | | | | | | |
| | [I1K689_SOYBN] | | | | | | | | |
| I1K6H2 | Uncharacterized protein | 8.47 | 1 | 2 | 7 | 8 | 921 | 104.2 | 6.38 |
| | OS = Glycine max PE = 4 SV = 2 - | | | | | | | | |
| | [I1K6H2_SOYBN] | | | | | | | | |
| I1KAD3 | Uncharacterized protein | 2.90 | 1 | 2 | 2 | 2 | 723 | 79.9 | 6.67 |
| | OS = Glycine max PE = 4 SV = 2 - | | | | | | | | |
| | [I1KAD3_SOYBN] | | | | | | | | |
| I1KL73 | Uncharacterized protein | 3.59 | 2 | 1 | 1 | 1 | 473 | 51.5 | 5.34 |
| | OS = Glycine max PE = 4 SV = 2 - | | | | | | | | |
| | [I1KL73_SOYBN] | | | | | | | | |
| I1KUB2 | Uncharacterized protein | 2.24 | 2 | 1 | 1 | 1 | 580 | 65.2 | 9.35 |
| | OS = Glycine max PE = 4 SV = 2 - | | | | | | | | |
| | [I1KUB2_SOYBN] | | | | | | | | |
| I1KVT5 | Uncharacterized protein | 16.18 | 1 | 2 | 2 | 2 | 136 | 15.5 | 6.01 |
| | OS = Glycine max PE = 4 SV = 2 - | | | | | | | | |
| | [I1KVT5_SOYBN] | | | | | | | | |
| I1L362 | Uncharacterized protein | 12.13 | 5 | 2 | 13 | 14 | 1336 | 147.8 | 6.16 |
| | OS = Glycine max PE = 4 SV = 2 - | | | | | | | | |
| | [I1L362_SOYBN] | | | | | | | | |
| I1L7G8 | Uncharacterized protein | 13.92 | 5 | 1 | 1 | 1 | 79 | 8.5 | 9.04 |
| | OS = Glycine max PE = 4 SV = 2 - | | | | | | | | |
| | [I1L7G8_SOYBN] | | | | | | | | |
| I1LFL5 | Uncharacterized protein | 2.69 | 1 | 2 | 2 | 2 | 1151 | 127.2 | 5.76 |
| | OS = Glycine max PE = 4 SV = 2 - | | | | | | | | |
| | [I1LFL5_SOYBN] | | | | | | | | |
| I1LHP6 | Uncharacterized protein | 27.73 | 1 | 10 | 18 | 25 | 69.6 | 81.7 | 5.34 |
| | OS = Glycine max PE = 4 SV = 2 - | | | | | | | | |
| | [I1LHP6_SOYBN] | | | | | | | | |
| I1LJ01 | Uncharacterized protein | 30.65 | 1 | 1 | 2 | 2 | 124 | 12.5 | 5.53 |
| | OS = Glycine max PE = 4 SV = 2 - | | | | | | | | |
| | [I1LJ01_SOYBN] | | | | | | | | |
| I1LJG9 | Uncharacterized protein | 1.06 | 1 | 1 | 1 | 1 | 756 | 83.8 | 5.77 |
| | OS = Glycine max PE = 4 SV = 2 - | | | | | | | | |
| | [I1LJG9_SOYBN] | | | | | | | | |
| I1LJU3 | Uncharacterized protein | 3.51 | 4 | 2 | 2 | 2 | 855 | 95.8 | 6.68 |
| | OS = Glycine max PE = 4 SV = 2 - | | | | | | | | |
| | [I1LJU3_SOYBN] | | | | | | | | |
| I1LRI7 | Uncharacterized protein | 5.08 | 2 | 2 | 2 | 2 | 532 | 59.2 | 6.84 |
| | OS = Glycine max PE = 4 SV = 2 - | | | | | | | | |
| | [I1LRI7_SOYBN] | | | | | | | | |
| I1LV12 | Uncharacterized protein | 10.54 | 4 | 5 | 5 | 5 | 607 | 68.3 | 9.04 |
| | OS = Glycine max PE = 4 SV = 2 - | | | | | | | | |
| | [I1LV12_SOYBN] | | | | | | | | |
| I1LWM2 | Uncharacterized protein | 20.76 | 2 | 3 | 3 | 3 | 419 | 46.2 | 4.86 |
| | OS = Glycine max PE = 4 SV = 2 - | | | | | | | | |
| | [I1LWM2_SOYBN] | | | | | | | | |
| I1LXQ1 | Uncharacterized protein | 10.76 | 3 | 5 | 5 | 5 | 725 | 79.5 | 7.75 |
| | OS = Glycine max PE = 4 SV = 2 - | | | | | | | | |
| | [I1LXQ1_SOYBN] | | | | | | | | |
| I1M2Y5 | Uncharacterized protein | 24.43 | 3 | 7 | 7 | 10 | 348 | 38.5 | 6.73 |
| | OS = Glycine max PE = 4 SV = 2 - | | | | | | | | |
| | [I1M2Y5_SOYBN] | | | | | | | | |
| I1M322 | Uncharacterized protein | 12.36 | 1 | 2 | 4 | 4 | 437 | 46.7 | 6.95 |
| | OS = Glycine max PE = 4 SV = 2 - | | | | | | | | |
| | [I1M322_SOYBN] | | | | | | | | |
| I1M903 | Uncharacterized protein | 1.79 | 2 | 1 | 1 | 1 | 670 | 69.1 | 5.86 |
| | OS = Glycine max PE = 4 SV = 2 - | | | | | | | | |
| | [I1M903_SOYBN] | | | | | | | | |
| I1MHT6 | Uncharacterized protein | 1.18 | 2 | 1 | 1 | 1 | 1187 | 134.5 | 5.24 |
| | OS = Glycine max PE = 4 SV = 2 - | | | | | | | | |
| | [I1MHT6_SOYBN] | | | | | | | | |
| I1MPM4 | Uncharacterized protein | 12.44 | 6 | 2 | 13 | 14 | 1326 | 146.4 | 6.09 |
| | OS = Glycine max PE = 4 SV = 2 - | | | | | | | | |
| | [I1MPM4_SOYBN] | | | | | | | | |
| I1N0U3 | Uncharacterized protein | 9.90 | 2 | 1 | 1 | 1 | 101 | 11.4 | 5.91 |
| | OS = Glycine max PE = 4 SV = 2 - | | | | | | | | |
| | [I1N0U3_SOYBN] | | | | | | | | |
| I1N1B9 | Uncharacterized protein | 6.10 | 4 | 2 | 2 | 2 | 344 | 38.1 | 8.05 |
| | OS = Glycine max PE = 4 SV = 2 - | | | | | | | | |
| | [I1N1B9_SOYBN] | | | | | | | | |
| I1N334 | Uncharacterized protein | 2.49 | 2 | 1 | 1 | 1 | 321 | 36.3 | 6.74 |
| | OS = Glycine max PE = 4 SV = 2 - | | | | | | | | |
| | [I1N334_SOYBN] | | | | | | | | |
| I1NAI0 | Uncharacterized protein | 15.07 | 1 | 2 | 2 | 2 | 146 | 16.2 | 5.00 |
| | OS = Glycine max PE = 4 SV = 2 - | | | | | | | | |
| | [I1NAI0_SOYBN] | | | | | | | | |
| Q8W1A3 | Uncoupling protein 1b | 6.64 | 8 | 1 | 1 | 1 | 241 | 25.7 | 9.60 |
| | (Fragment) OS = Glycine max | | | | | | | | |
| | PE = 3 SV = 1 - | | | | | | | | |
| | [Q8W1A3_SOYBN] | | | | | | | | |
| I1K3K3 | Urease OS = Glycine max PE = 3 | 40.05 | 5 | 22 | 22 | 46 | 839 | 90.3 | 6.07 |
| | SV = 1 - [I1K3K3_SOYBN] | | | | | | | | |
| C6TNK1 | Uricase OS = Glycine max PE = 2 | 18.12 | 4 | 4 | 4 | 5 | 309 | 35.1 | 8.29 |
| | SV = 1 - [C6TNK1_SOYBN] | | | | | | | | |
| D7EYG6 | V-H(+)-ATPase subunit A | 16.37 | 3 | 8 | 8 | 9 | 623 | 68.7 | 5.58 |
| | OS = Glycine max PE = 2 SV = 1 - | | | | | | | | |
| | [D7EYG6_SOYBN] | | | | | | | | |
| I1LJ94 | V-type proton ATPase subunit a | 2.93 | 2 | 2 | 2 | 2 | 818 | 92.9 | 6.18 |
| | OS = Glycine max PE = 3 SV = 1 - | | | | | | | | |
| | [I1LJ94_SOYBN] | | | | | | | | |
| Q02243 | Wound-induced protein | 12.75 | 3 | 1 | 1 | 1 | 102 | 11.1 | 7.71 |
| | (Fragment) OS = Glycine max | | | | | | | | |
| | GN = WIN PE = 2 SV = 1 - | | | | | | | | |
| | [WIN_SOYBN] | | | | | | | | |
| I1MSU1 | Xylose isomerase OS = Glycine | 9.17 | 3 | 4 | 4 | 4 | 480 | 54.2 | 6.43 |
| | max PE = 3 SV = 1 - | | | | | | | | |
| | [I1MSU1_SOYBN] | | | | | | | | |

### SUMMARY OF THE INVENTION

A food supplement composition arranged for human consumption comprising a plurality of ingredients, all from peanut. The invention provides a process for obtaining juice and powder from peanut, wherein the seed of peanut is grinded and centrifuged to make juice and powder. Total 837 proteins found in peanut. Among them, 296 are uncharacterized and 35 are putative uncharacterized new found KH good healthy proteins synthesized by KH good healthy cells. Total 682 proteins found in cashew. Among them, 490 are uncharacterized new found KH good healthy proteins synthesized by KH good healthy cells. Total 1,223 proteins found in edamame. Among them, 679 are uncharacterized and 164 are putative uncharacterized new found KH good healthy proteins synthesized by KH good healthy cells.

### BRIEF DESCRIPTION OF THE DRAWINGS

FIG. 1 flow chart of peanut process

FIG. 2 SDS-Page of supernatant of KH 169 Peanut

FIG. 3 SDS-Page of paste of KH 169 Peanut

FIG. 4 flow chart of cashew process

FIG. 5 SDS-Page of supernatant of KH 170 cashew

FIG. 6 SDS-Page of paste of KH 170 cashew

FIG. 7 flow chart of edamame process

FIG. 8 SDS-Page of supernatant of KH 169 Edamame

FIG. 9 SDS-Page of paste of KH 169 Edamame

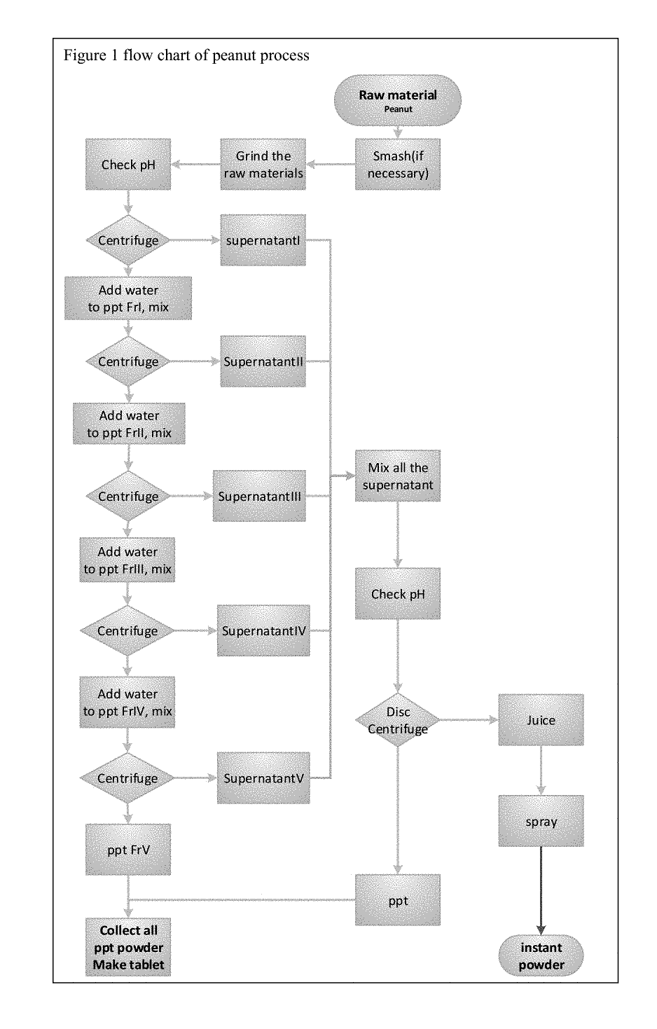

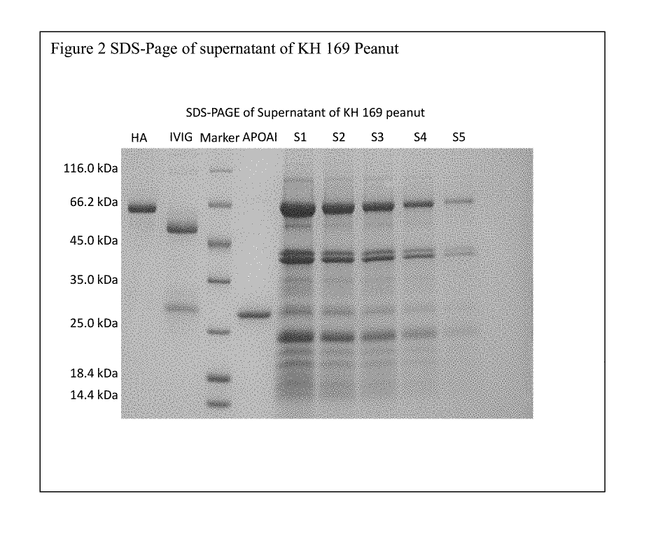

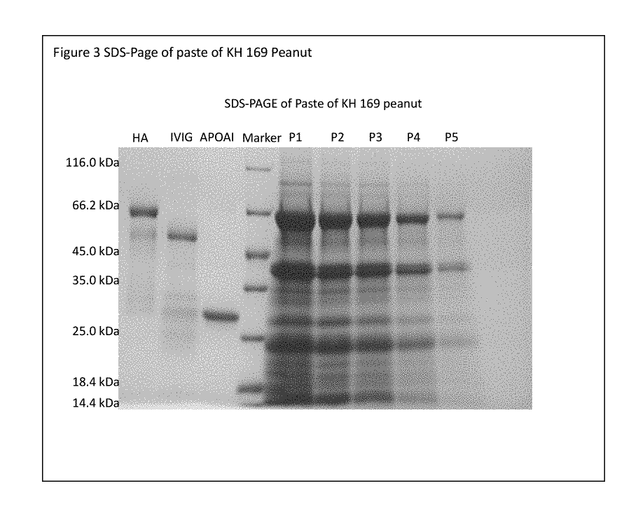

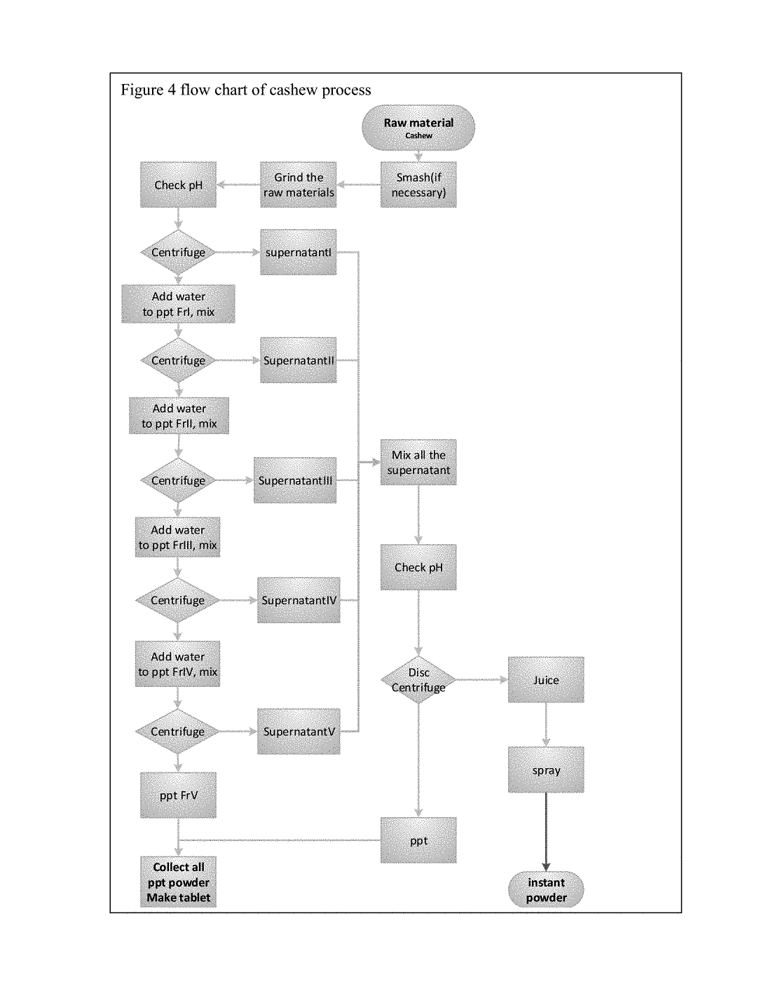

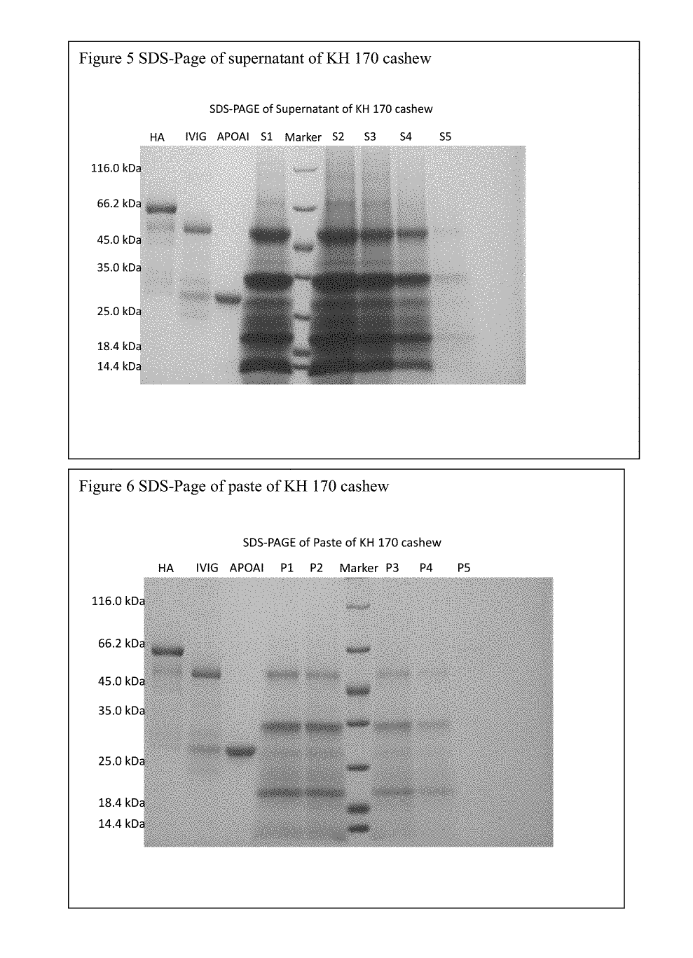

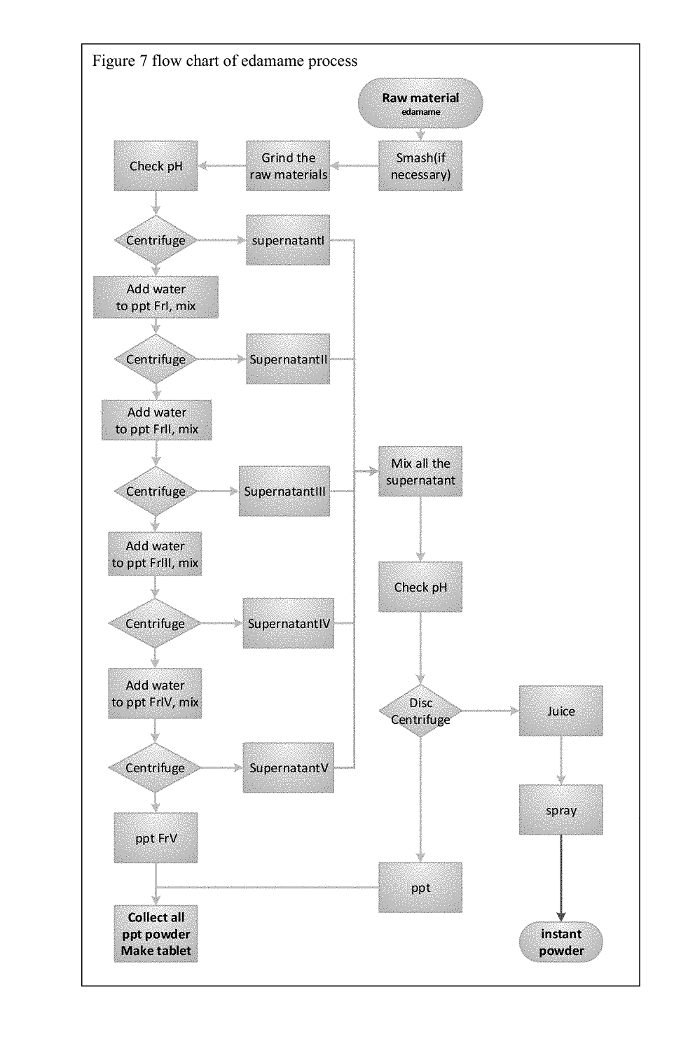

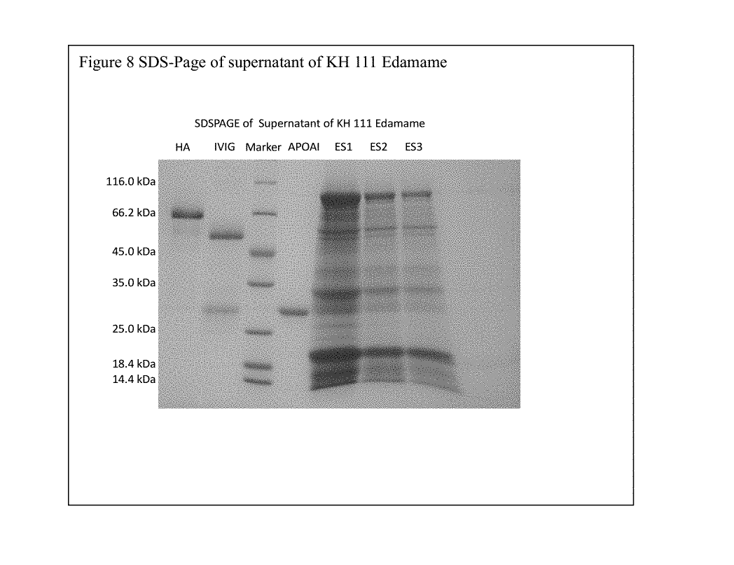

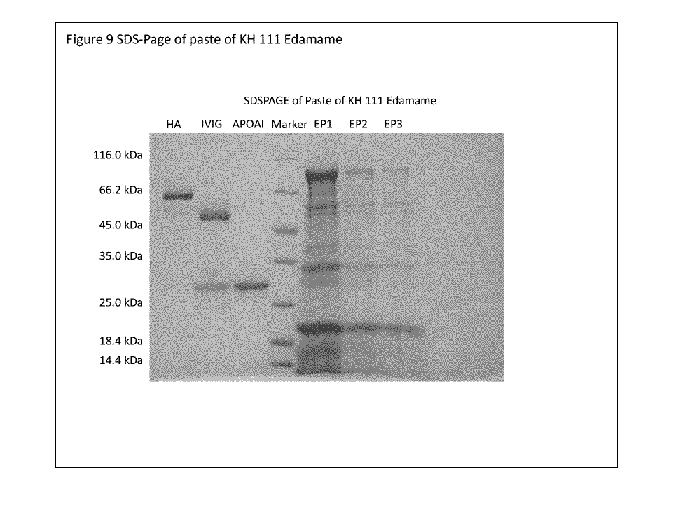

Table 1 cell count in the supernatant and paste of KH 169 peanut

Table 2 cell count in the supernatant and paste of KH 170 cashew

Table 3 cell count in the supernatant and paste of KH 169 edamame

### DETAILED DESCRIPTION OF THE INVENTION

### Peanut

The present invention describes a method for the production of a food supplement including juice and powder from peanut.
The peanut is treated immediately after harvest.
Wash the peanut in cleaning bath at 5 about 0-100 Celsius.
Smash the whole peanut in grinder to cut the thick stem at about 0-100 Celsius. Collect all the raw materials.
Grind the raw materials in superfine mill at about 0-100 Celsius. Collect the raw juice.
Check the pH of the raw juice, about 1.5-10
Centrifuge the raw juice at low to high rpm with normal centrifuge at about −30 to 30 Celsius. Collect the precipitate (“ppt”) and supernatant.
Add 2-4 volume water to ppt. Mix about 10-100 minutes and centrifuge again at low to high rpm with normal centrifuge at about −30 Celsius to 60. Collect the ppt 15 and supernatant.
Add 1-2 volume water to it. Mix about 10-100 minutes and centrifuge again at low to high rpm with normal centrifuge at about −30 to 60 Celsius. Collect the ppt and supernatant.
Mix all the supernatant for 10-100 minutes at about 0-100 Celsius.
Check the supernatant pH, about 1.5 to 10
Centrifuge the supernatant juice at moderate to high rpm with a disc centrifuge at about −30 to 60 Celsius, collect the juice.
Check the juice pH, about. 2.5 to 10 pH

1. If making juice

Homogenizing the juice at 40-60 MPa.

Sterilize the juice at 130-140 Celsius for 5-20 seconds with a pipe sterilizer.

Bottle the clear juice at 25-35 Celsius. Take sample and check the pH

2. If making powder

The juice is concentrated in Single-effect Falling Film Evaporator at 65-85 Celsius until the brix value is 30-50.

The concentrated juice is transfer to the Centrifugal Spray Dryer to get powder.

3. Mix all the 5 ppt to make capsules, tablet or powder in pouch.

TABLE 1
cell count in the supernatant and paste of KH 169 peanut

| | Supernatant (/mL) | | | | |
|---|---|---|---|---|---|
| | S1 | S2 | S3 | S4 | S5 |
| KH 169 Peanut | 7,690,000 | 2,110,000 | 226,000 | 5,540,000 | 5,380,000 |
| | Paste (/mL) | | | | |
| | P1 | P2 | P3 | P4 | P5 |
| KH 169 Peanut | 659,000,000 | 1,324,000,000 | 1,320,000,000 | 439,000,000 | 116,000,000 |

### Cashew

The present invention describes a method for the production food supplement of juice and powder from cashew.
The cashew is treated immediately after harvest.
Wash the cashew in cleaning bath at 5 about 0-100 Celsius.
Smash the whole cashew in grinder to cut the thick stem at about 0-100 Celsius. Collect all the raw materials.
Grind the raw materials in superfine mill at about 0-100 Celsius. Collect the raw juice.
Check the pH of the raw juice, about 1.5-10
Centrifuge the raw juice at low to high rpm with normal centrifuge at about −30 to 30 Celsius. Collect the precipitate (“ppt”) and supernatant.
Add 2-4 volume water to ppt. Mix about 10-100 minutes and centrifuge again at low to high rpm with normal centrifuge at about −30 Celsius to 60. Collect the ppt 15 and supernatant.
Add 1-2 volume water to it. Mix about 10-100 minutes and centrifuge again at low to high rpm with normal centrifuge at about −30 to 60 Celsius. Collect the ppt and supernatant.
Mix all the supernatant for 10-100 minutes at about 0-100 Celsius.
Check the supernatant pH, about 1.5 to 10
Centrifuge the supernatant juice at moderate to high rpm with a disc centrifuge at about −30 to 60 Celsius, collect the juice.
Check the juice pH, about 1.5 to 10 pH

1. If making juice:

Homogenizing the juice at 40-60 MPa.

Sterilize the juice at 130-140 Celsius for 5-20 seconds with a pipe sterilizer.

Bottle the clear juice at 25-35 Celsius. Take sample and check the pH

2. If making powder:

The juice is concentrated in Single-effect Falling Film Evaporator at 65-85 Celsius until the brix value is 30-50.

The concentrated juice is transfer to the Centrifugal Spray Dryer to get powder.

Packaging the powder.

3. Mix all the 5 ppt to make tablets, capsules or powder in pouch.

TABLE 2
cell count in the supernatant and paste of KH 170 cashew

| | Supernatant (/mL) | | | | |
|---|---|---|---|---|---|
| | S1 | S2 | S3 | S4 | S5 |
| KH 170 cashew | 5,760,000 | 3,690,000 | 5,310,000 | 11,200,000 | 2,950,000 |
| | Paste (/mL) | | | | |
| | P1 | P2 | P3 | P4 | P5 |
| KH 170 cashew | 231,000,000 | 246,000,000 | 94,900,000 | 346,000,000 | 276,000,000 |

### Edamame

The present invention describes a method for the production of food supplement including juice and powder from edamame (young soy).
The edamame is treated immediately after harvest.
Wash the edamame in cleaning bath at 5 minutes about 0-100 Celsius.
Smash the whole edamame in grinder to cut the thick stem at about 0-100 Celsius. Collect all the raw materials.
Grind the raw materials in superfine mill at about 0-100 Celsius. Collect the raw juice.
Check the pH of the raw juice, about 1.5-10
Centrifuge the raw juice at low to high rpm with normal centrifuge at about −30 to 30 Celsius. Collect the precipitate (“ppt”) and supernatant.
Add 2-4 volume water to ppt. Mix about 10-100 minutes and centrifuge again at low to high rpm with normal centrifuge at about −30 Celsius to 60. Collect the ppt 1 and supernatant.
Add 1-2 volume water to it. Mix about 10-100 minutes and centrifuge again at low to high rpm with normal centrifuge at about −30 to 60 Celsius. Collect the ppt and supernatant.
Mix all the supernatant for 10-100 minutes at about 0-100 Celsius.
Check the supernatant pH, about 1.5 to 10
Centrifuge the supernatant juice at moderate to high rpm with a disc centrifuge at about −30 to 60 Celsius, collect the juice.
Check the juice pH, about 1.5 to 10 pH

1. If making juice

Homogenizing the juice at 40-60 MPa.

Sterilize the juice at 130-140 Celsius for 5-20 seconds with a pipe sterilizer.

Bottle the clear juice at 25-35 Celsius. Take sample and check the pH

2. If making powder

The juice is concentrated in Single-effect Falling Film Evaporator at 65-85 Celsius until the brix value is 30-50.

The concentrated juice is transfer to the Centrifugal Spray Dryer to get powder.

Packaging the powder.

3. Mix all the 5 ppt to make capsules, tablets or powder in pouch.

TABLE 3
cell count in the supernatant and paste of KH 169 edamame

| | Supernatant (/mL) | | | | |
|---|---|---|---|---|---|
| | S1 | S2 | S3 | S4 | S5 |
| KH 111 | 7,690,000 | 2,110,000 | 226,000 | 5,540,000 | 5,380,000 |
| Edamame | | | | | |
| | Paste (/mL) | | | | |
| | P1 | P2 | P3 | P4 | P5 |
| KH 111 | 659,000,000 | 1,324,000,000 | 1,320,000,000 | 439,000,000 | 116,000,000 |
| Edamame | | | | | |

1. A food supplement composition arranged for human consumption comprising a plurality of ingredients, all from peanut. The invention provides a process for obtaining juice and powder from peanut, wherein the seed of peanut is grinded and centrifuged to make juice and powder. Total 837 proteins found in peanut. Among them, 296 are uncharacterized and 35 are putative uncharacterized new found KH good healthy proteins synthesized by KH good healthy cells.

2. Method of claim 1, wherein juice is mixed beverage that will contain certain number of cells

3. Method of claim 2, wherein Powder is mixed food supplement that will also contain certain number of cells

4. Method of claim 2, peanut has KH GOOD HEALTHY CELLS in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

5. Method of claim 2, in peanut contains proteins whose molecular weight are similar to the molecular weight of the human proteins of human albumin, immunoglobulin and APOA-1.

6. Method of claim 2, living cells are found in this dried powder and beverages

7. A food composition arranged for human consumption comprising a plurality of ingredients, all from cashew. The invention provides a process for obtaining food supplement of juice and powder from cashew, wherein the seed of cashew is grinded and centrifuged to make juice and powder. Total 682 proteins found in cashew, among them, 490 are uncharacterized new found KH good healthy proteins synthesized by KH good healthy cells.

8. Method of claim 6, wherein juice is mixed beverage that will contain certain number of cells

9. Method of claim 7, wherein Powder is mixed food supplement that will also contain certain number of cells

10. Method of claim 7, cashew has KH GOOD HEALTHY CELLS in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

11. Method of claim 7, in cashew contains proteins whose molecular weights are similar to the molecular weight of the human proteins of human albumin, immunoglobulin and APOA-1.

12. Method of claim 7, living cells are found in this dried powder and beverages

13. A food supplement composition arranged for human consumption comprising a plurality of ingredients, all from edamame. The invention provides a process for obtaining juice and powder from edamame, wherein the seed of edamame is grinded and centrifuged to make juice and powder. Total 1,223 proteins found in edamame, among them, 679 are uncharacterized and 164 are putative uncharacterized new found KH good healthy proteins synthesized by KH good healthy cells.

14. Method of claim 13, wherein juice is mixed beverage that will contain certain number of cells

15. Method of claim 14, wherein Powder is mixed food supplement that will also contain certain number of cells

16. Method of claim 14, edamame has KH GOOD HEALTHY CELLS in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

17. Method of claim 14, in edamame contains proteins whose molecular weight are similar to the molecular weight of the human proteins of human albumin, immunoglobulin and APOA-1.

18. Method of claim 14, living cells are found in this dried powder and beverages
